(19) United States
(12) Patent Application Publication
(10) Pub. No.: US 2026/0248927 A1
(43) Pub. Date: Aug. 27, 2026

(54) TARGETED PROTEIN DEGRADATION USING BIFUNCTIONAL COMPOUNDS THAT BIND UBIQUITIN LIGASE AND TARGET MCL-1 PROTEIN

(71) Applicant: Captor Therapeutics S.A., Wroclaw (PL)

(72) Inventors: Sylvain COTTENS, Witterswil (CH); Tomasz TOMCZYK, Kamieniec Wroclawske (PL); Andrzej TRACZ, Wilczyce (PL); Michal Jerzy WALCZAK, Wroclaw (PL); Karolina WÓJCIK, Wroclaw (PL)

(21) Appl. No.: 19/135,369

(22) PCT Filed: Dec. 6, 2023
(86) PCT No.: PCT/EP2023/084601

§ 371 Date: Jun. 3, 2025

(30) Foreign Application Priority Data
Dec. 6, 2022 (WO) PCT/PC2022/000068

(51) Int. Cl. A61K 47/55; A61K 45/06; A61P 35/02

(52) U.S. Cl. CPC A61K 47/55; A61K 45/06; A61P 35/02

(57) ABSTRACT

A compound of formula (I) [MCL-1 ligand moiety]-[linker]-[ligase ligand moiety](I) or a salt, solvate, hydrate, isomer or prodrug thereof, wherein [MCL-1 ligand moiety] is a compound of Formula (A) or wherein [MCL-1 ligand moiety] is a compound of Formula (A1), (A2), (A3) or (A4):, and its use in the treatment of cancer.

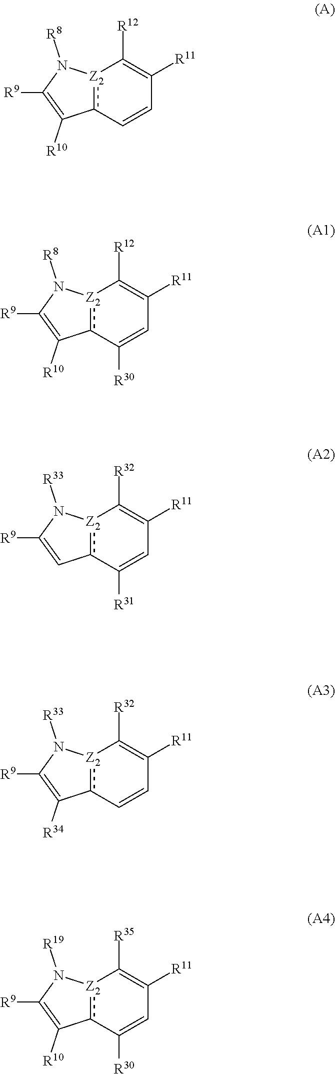

### FIELD OF THE INVENTION

The present invention relates to bifunctional compounds which can bind to a ubiquitin ligase and also to a target protein, such that the target protein is placed in proximity to the ubiquitin ligase in order to induce its degradation.

### BACKGROUND

The Ubiquitin-Proteasome System (UPS) is responsible for the maintenance of healthy and well-balanced proteome. In the process of ubiquitination, ubiquitin units are covalently attached to the protein, forming a polyubiquitin chain, which marks the protein for degradation via the proteasome. Ubiquitination is central to the regulation of nearly all cellular processes and is also tightly regulated itself. Ubiquitin ligases facilitate ubiquitination of different proteins in vivo and contribute to precise regulation of the system. Upon recognition, the ubiquitin ligases mediate the attachment of ubiquitin moieties to the target protein, which label it for degradation by the proteasome.

The idea of selective target protein degradation (TPD) by modulation of UPS was first described in 1999 (US2002173049 A1 (PROTEINIX INC) 21 Nov. 2002). One approach to TPD is by the use of bifunctional molecules that bind the ubiquitin ligase and the target protein simultaneously, allowing for efficient ubiquitin transfer to the latter. This concept was first described by Sakamoto K M et al. (Proc Natl Acad Sci USA. 2001 Jul. 17; 98(15):8554-9) and more recently reviewed by Burslem G M and Crews C M (Cell. 2020 Apr. 2; 181(1):102-114).

Oncogenic stress, such as DNA damage, may result in programmed cell death, the cellular response meant to prevent the oncogenic transformation. This mechanism depends on an interplay between pro-apoptotic and anti-apoptotic Bcl-2 proteins, and the balance of these proteins is essential for the proper functioning of the cell.

BCL-2, BCL-xL and MCL-1 are BH3-domain-containing anti-apoptotic proteins. These proteins bind to effector Bcl-2 proteins Bak and Bax (via their BH3 domains), preventing their pro-apoptotic activity. Inhibition of BH3 domain—BH3 pocket binding interface is a well-known approach to cancer therapy (Leber B, Kale J, Andrews D W. Cancer Discov. 2018 December; 8(12):1511-1514).

High expression of induced myeloid leukaemia cell differentiation protein (MCL-1) is observed in many human cancers and is associated with resistance to cytotoxic drugs. Research shows that inhibition of MCL-1 protein in some malignancies leads to the release of pro-apoptotic proteins and induction of apoptosis. Therefore, targeting MCL-1 can be applied as a therapeutic strategy in these types of cancer which are MCL-1 dependent, such as multiple myeloma, acute myeloid leukaemia, chronic myeloid leukaemia, B-cell acute lymphoblastic leukaemia, hepatocellular carcinoma and non-small cell lung cancers. This concept was confirmed in vitro and in vivo (Tron A E et al. Nat Commun. 2018 Dec. 17; 9(1):5341). Also, treatment with Bcl-2 inhibitors and MEK inhibitors often elicits MCL-1 dependence and subsequent inactivation of MCL-1 results in synthetic lethality (Leber B, Kale J, Andrews D W. Cancer Discov. 2018 December; 8(12):1511-1514). As demonstrated by Montero, J. et al. (Nat. Commun. 10, 5157 (2019)) and Sale, M. J. et al. (Nat. Commun. 10, 5167 (2019)), MCL-1 is a driver of adaptive survival in tumor cells treated with oncogene targeted therapies, therefore MCL-1 targeting drugs are likely to overcome cancer resistance to these therapeutics.

In parallel to the efforts focused on inhibition of MCL-1, targeted degradation appears as an attractive therapeutic alternative. Both Papatzimas et al. (J. Med. Chem. 2019, 62, 11, 5522-5540) and Wang Z et al. (J. Med. Chem. 2019, 62, 17, 8152-8163) have demonstrated degradation of the MCL-1 protein. However, the potency of reported compounds in terms of cellular degradation and the ability to induce apoptosis remains suboptimal. Therefore, alternative chemotypes with improved potency are needed to develop therapeutically applicable MCL-1 degraders.

One of the challenges in the development of MCL-1 targeted therapeutics is related to safety, as MCL-1 has been shown to be essential for cardiac homeostasis in adult murine models, and the absence of MCL-1 led to loss of cardiomyocytes. Some clinical trials involving MCL-1 inhibitors are currently on clinical hold to evaluate a safety signal for cardiac toxicity (Wei A H et al. Blood Rev. 2020 November; 44: 100672).

### SUMMARY OF INVENTION

In accordance with a first aspect of the invention, there is provided a compound of formula (I)

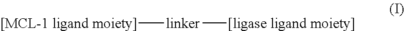

or a salt, solvate, hydrate, isomer or prodrug thereof,
wherein [ligase ligand moiety] is:

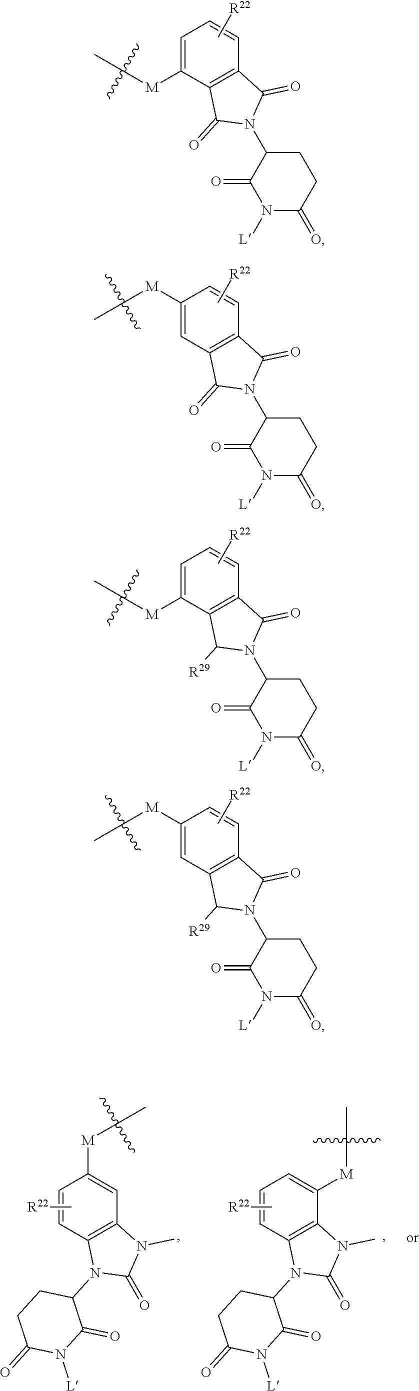

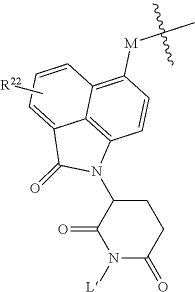

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   R²⁹ is hydrogen or Me
   L′ is hydrogen, alkyl, benzyl, acetyl or pivaloyl;
wherein [MCL-1 ligand moiety] is a compound of Formula (A1), (A2), (A3) or (A4):

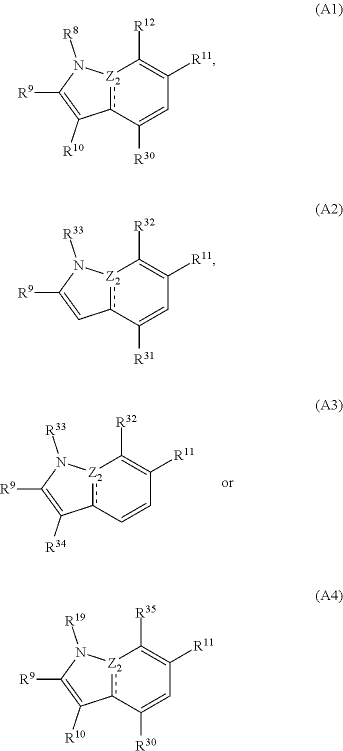

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹² is

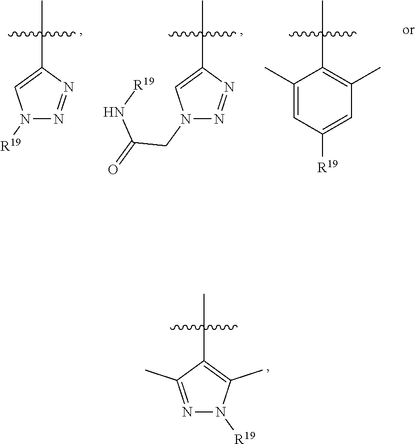

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

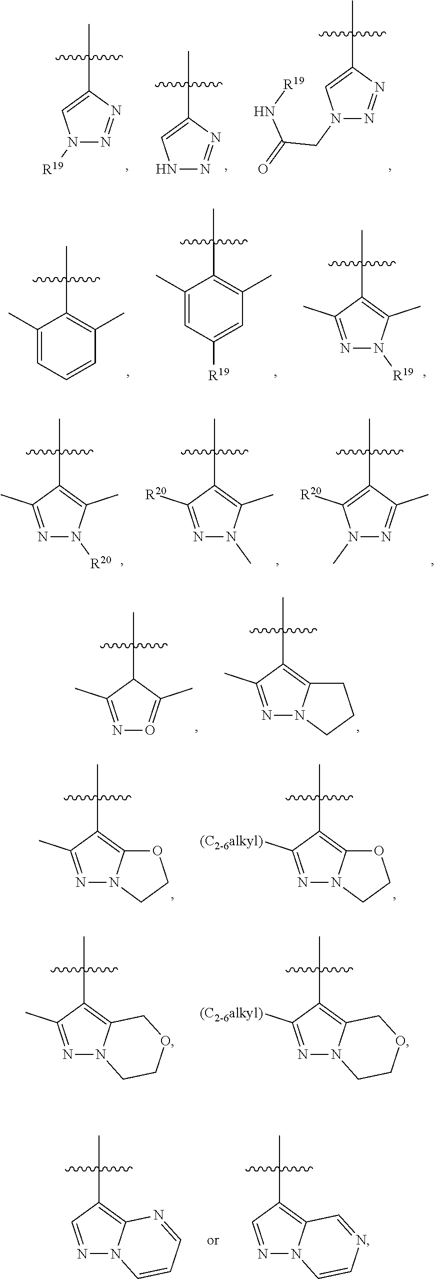

wherein R²⁰ is Me, —CH₂—OMe, —(CH₂CH₂O)ₚ(C₁-C₆ alkyl), or —CH₂—O-bromobenzaldehyde, wherein p is 1-5;
   R³⁴ is C₂₋₅alkyl-O—R¹³ or —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
   R³⁵ is

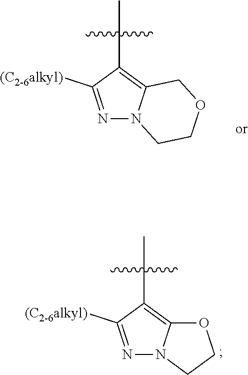

and
R¹⁹ is a bond connected to R¹⁴ of the linker, wherein each of Formula (A1), Formula (A2), Formula (A3) and Formula (A4) contains a single R¹⁹;
and wherein [linker] has the following formula

R¹⁴-R¹⁵-R¹⁶-R¹⁷-R¹⁸
wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R⁵ is cycloalkyl, heterocycloalkyl, aryl, heteroaryl, C₁₋₆ alkyl-NH—, —C₁₋₆-alkyl-N(C₁₋₆ alkyl)-, -cycloalkyl-NH—, -heterocycloalkyl-NH— or is absent
   R¹⁶ is —C₁₋₆alkyl, —C(O)—C₁₋₆alkyl-, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent
   wherein at least one of R¹⁴-R¹⁸ is present.

In some embodiments, [ligase ligand moiety] is:

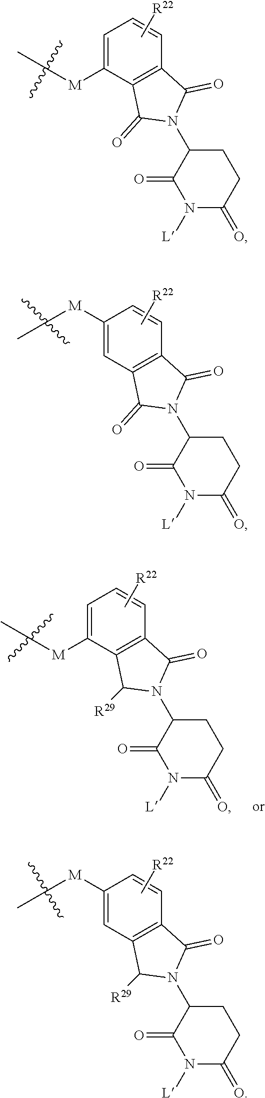

In some embodiments, R²⁹ is Me. In other embodiments, R²⁹ is hydrogen.

In some embodiments, [ligase ligand moiety] is

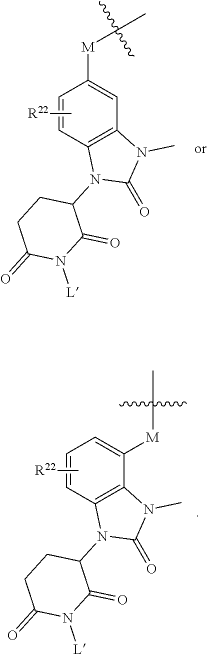

In other embodiments, [ligase ligand moiety] is

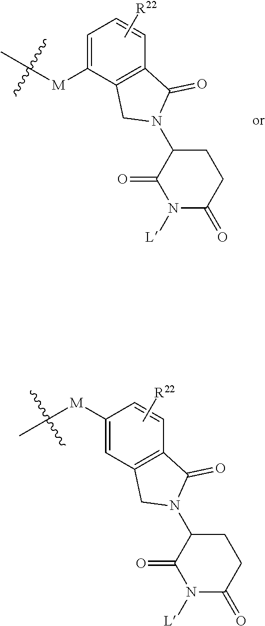

In some embodiments, R²² is hydrogen, halogen, —OMe, —NH₂, —NHMe, —NMe₂, or piperidine. In some such embodiments, R²² is hydrogen, —OMe, —NH₂, —NHMe, —NMe₂, or piperidine. In some embodiments, R²² is hydrogen.

In some embodiments, L′ is hydrogen.

In some embodiments, M is O or NH, or is absent.

In some embodiments, [ligase ligand moiety] is

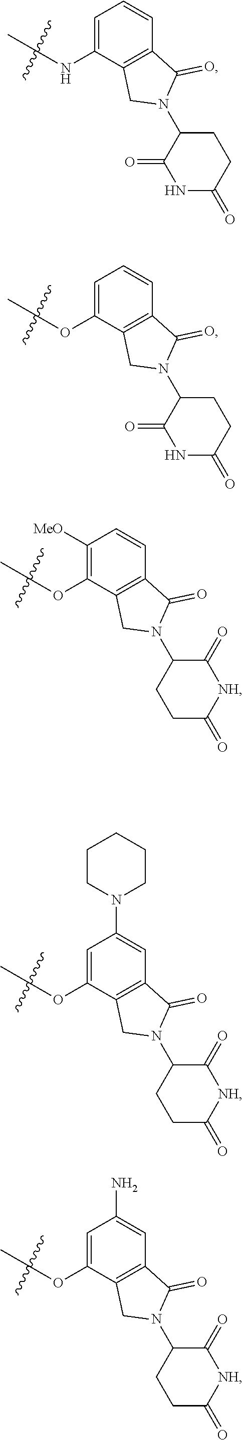 [SMILES: *Nc1cccc2c1CN(C1CCC(=O)NC1=O)C2=O ; *Oc1cccc2c1CN(C1CCC(=O)NC1=O)C2=O ; *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cc(N)cc2c1CN(C1CCC(=O)NC1=O)C2=O]

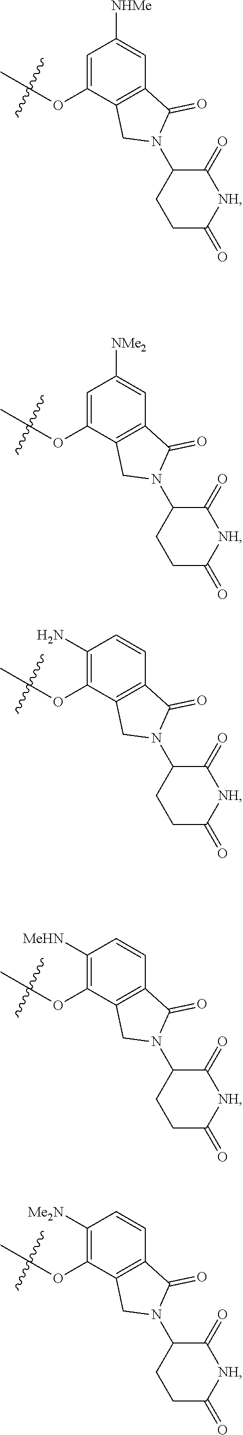 [SMILES: *Oc1cc(NC)cc2c1CN(C1CCC(=O)NC1=O)C2=O ; *Oc1cc(N(C)C)cc2c1CN(C1CCC(=O)NC1=O)C2=O ; *Oc1c(N)ccc2c1CN(C1CCC(=O)NC1=O)C2=O ; *Oc1c(NC)ccc2c1CN(C1CCC(=O)NC1=O)C2=O ; *Oc1c(N(C)C)ccc2c1CN(C1CCC(=O)NC1=O)C2=O]

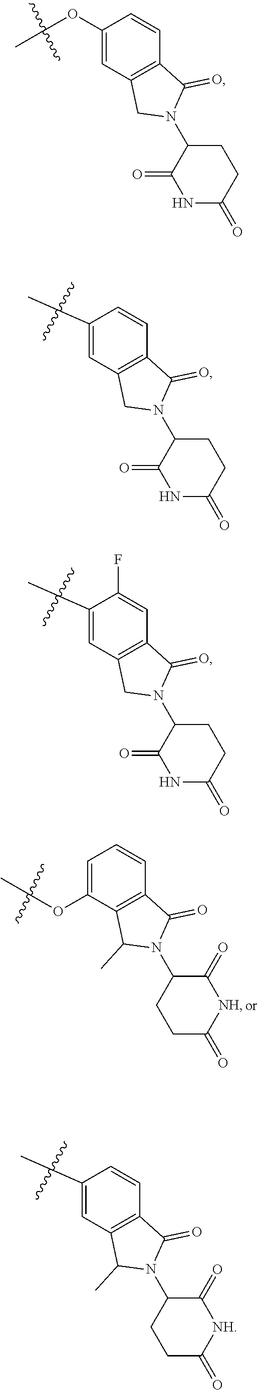 [SMILES: *Oc1ccc2c(c1)CN(C1CCC(=O)NC1=O)C2=O ; *c1ccc2c(c1)CN(C1CCC(=O)NC1=O)C2=O ; *c1cc2c(cc1F)C(=O)N(C1CCC(=O)NC1=O)C2 ; *Oc1cccc2c1C(C)N(C1CCC(=O)NC1=O)C2=O ; *c1ccc2c(c1)C(C)N(C1CCC(=O)NC1=O)C2=O]

In some embodiments, [ligase ligand moiety] is

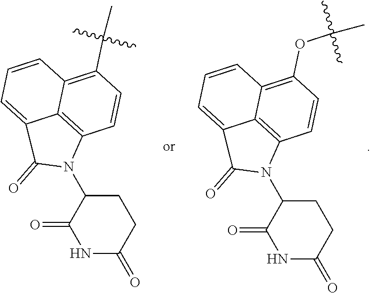 [SMILES: *c1ccc2c3c(cccc13)C(=O)N2C1CCC(=O)NC1=O ; *Oc1ccc2c3c(cccc13)C(=O)N2C1CCC(=O)NC1=O]

In some embodiments, [ligase ligand moiety] is

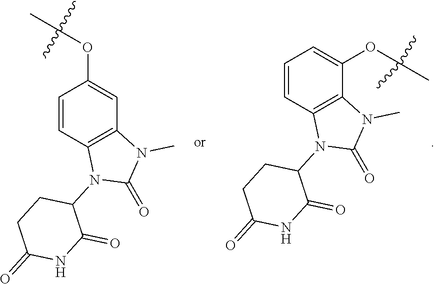 [SMILES: *Oc1ccc2c(c1)n(C)c(=O)n2C1CCC(=O)NC1=O ; *Oc1cccc2c1n(C)c(=O)n2C1CCC(=O)NC1=O]

In some embodiments,
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(C₂H₄—O)ₓ, —(C₃H₆—O)ₓ, or is absent
R¹⁸ is —C₁₋₆ alkyl, cycloalkyl, —CH₂—NH—C(O)—, heterocycloalkyl, or is absent.

In some embodiments, R¹⁴ is —C₁₋₆ alkyl, —C₂₋₆ alkenyl, —C₂₋₆ alkynyl, —C(O)—, —SO₂— or is absent. In some embodiments, R¹⁴ is —C₁₋₆ alkyl.

In some embodiments, R¹⁵ is cycloalkyl, heterocycloalkyl, aryl, heteroaryl, —C₁₋₆ alkyl-NH—, -cycloalkyl-NH— or is absent. In some such embodiments, R¹⁵ is heterocycloalkyl or is absent. In some embodiments, R¹⁵ is piperazine, bridged piperazine, piperazine N-oxide, piperazine cation,

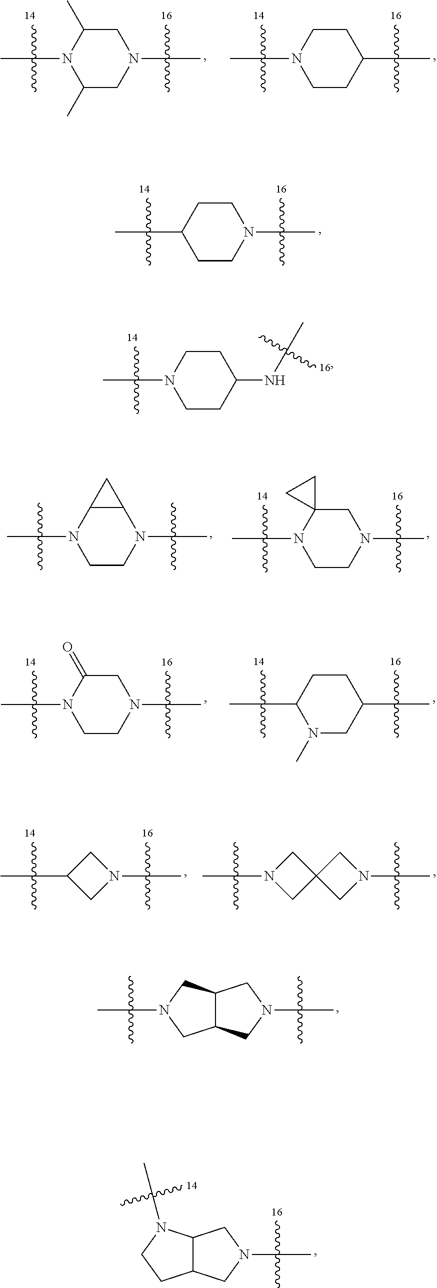

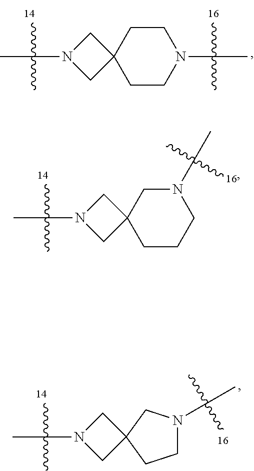 [SMILES: *N1CCC2(CC1)CN(*)C2 ; *N1CCCC2(C1)CN(*)C2 ; *N1CCC2(C1)CN(*)C2]

or is absent;
wherein [symbol] indicates attachment to R¹⁴ and [symbol] indicates attachment to R¹⁶.

In some embodiments, R¹⁵ is piperazine,

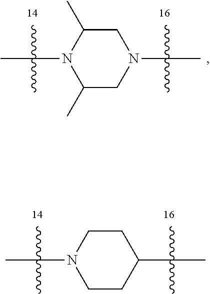

or is absent.

In some embodiments, R¹⁶ is —C₁₋₆ alkyl, —CH₂—C(O)—NH—, —CH₂—C(O)—, —C(O)— or is absent.

In some embodiments, R¹⁷ is absent.

In some embodiments, R¹⁸ is —C₁₋₆ alkyl, cyclobutyl, CH₂—NH—C(O)—, piperazine or is absent.

In some embodiments, [linker] is selected from

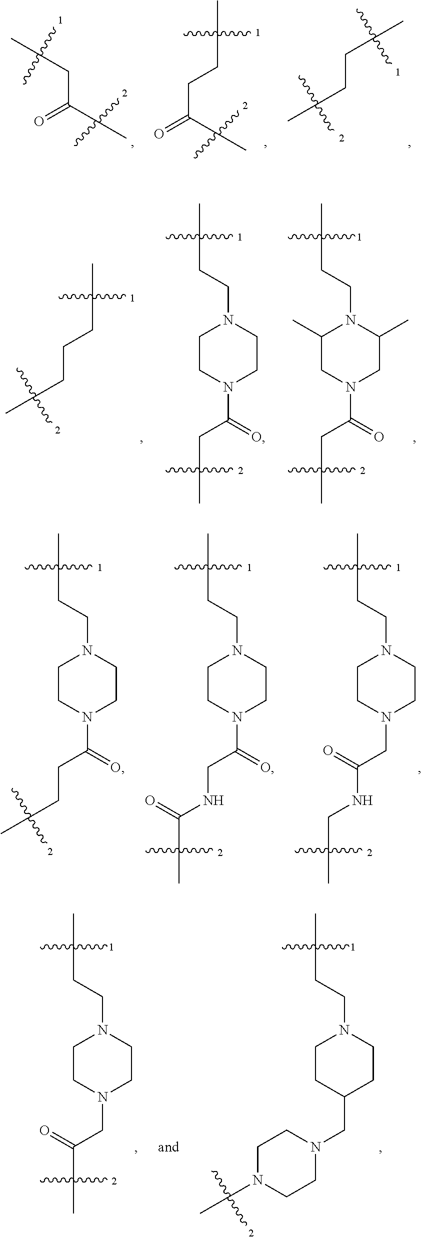

wherein
[symbol] indicates attachment to [MCL-1 ligand moiety] and
[symbol] indicates attachment to [ligase ligand moiety].

In some embodiments, [linker] is selected from

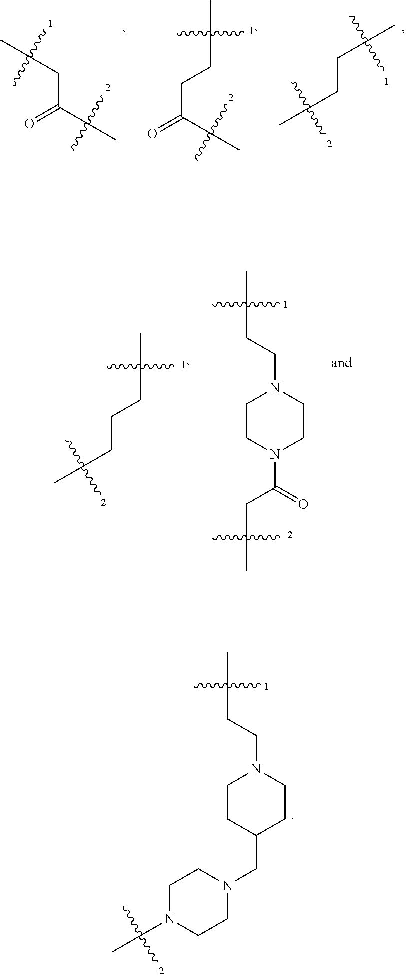

In some embodiments, [MCL-1 ligand moiety] is of Formula (A1):

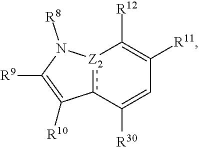

In some embodiments, [MCL-1 ligand moiety] is of Formula (A2):

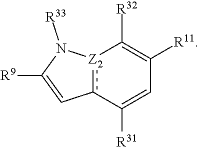

In some embodiments, [MCL-1 ligand moiety] is of Formula (A3):

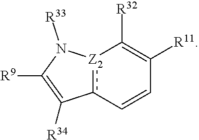

In some embodiments, [MCL-1 ligand moiety] is of Formula (A4):

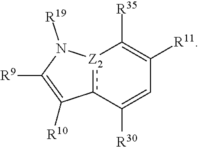

In some embodiments, in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments, in Formula (A1) R¹² is

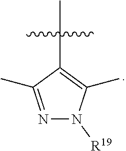

In some embodiments, in Formula (A4) R³⁵ is

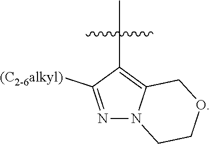

In some embodiments, in Formula (A2) R³¹ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments, in Formula (A3) R³⁴ is C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl. In some embodiments, Z₂ is C and [symbol] is a double bond.

In some embodiments, each R⁹ is independently —C(O)OH or —P(O)(OH)₂.

In some embodiments, R¹¹ is halogen.

In some embodiments, each R²⁰ is Me or —(CH₂CH₂O)₂Me.

In some embodiments, the C₁-C₆ alkyl substituted with morpholine or a piperazine is

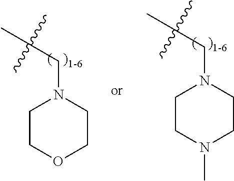

In some embodiments, [MCL-1 ligand moiety] is selected from:

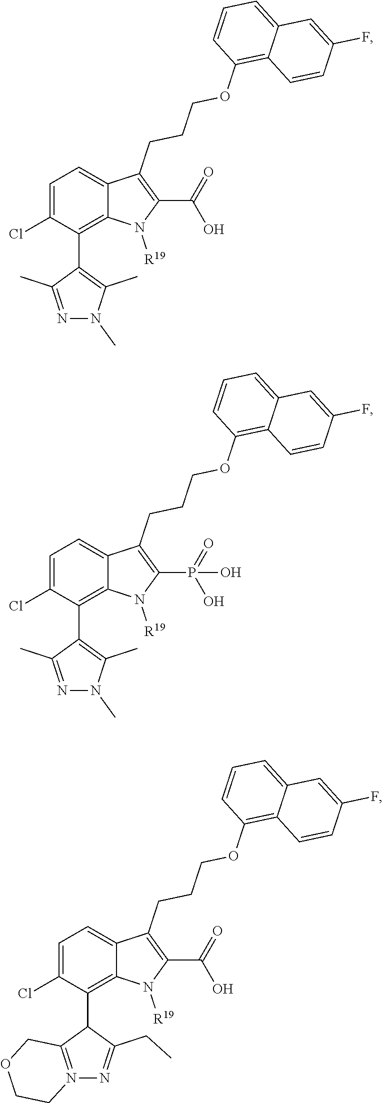

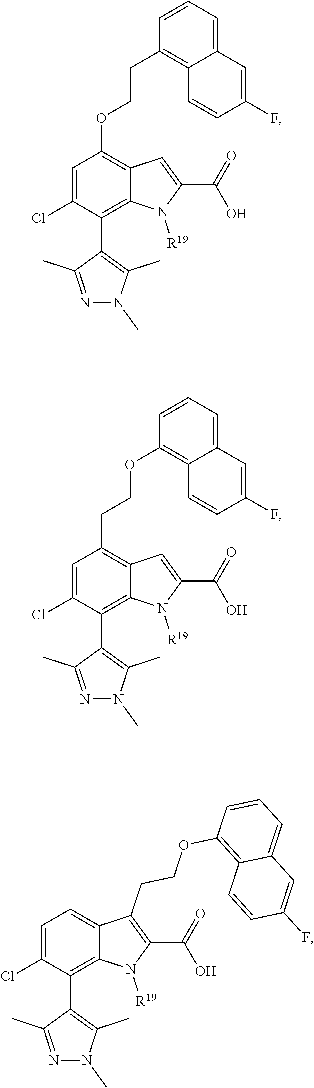

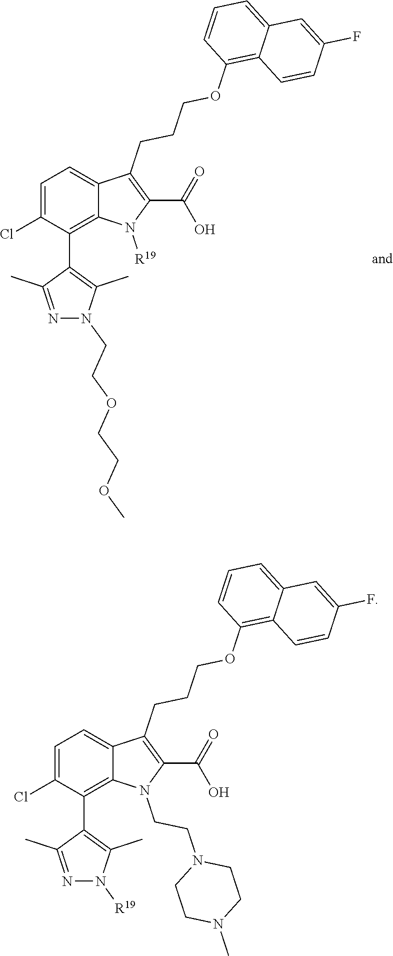

In some embodiments, the compound is selected from:

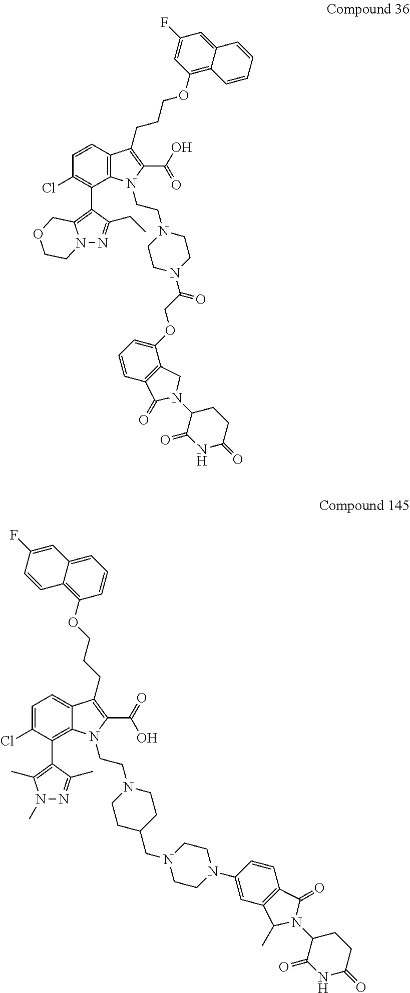 [SMILES: CCc1nn2c(c1-c1c(Cl)ccc3c(CCCOc4cc(F)cc5ccccc45)c(C(=O)O)n(CCN4CCN(C(=O)COc5cccc6c5CN(C5CCC(=O)NC5=O)C6=O)CC4)c13)COCC2 ; Cc1nn(C)c(C)c1-c1c(Cl)ccc2c(CCCOc3cccc4cc(F)ccc34)c(C(=O)O)n(CCN3CCC(CN4CCN(c5ccc6c(c5)C(C)N(C5CCC(=O)NC5=O)C6=O)CC4)CC3)c12]

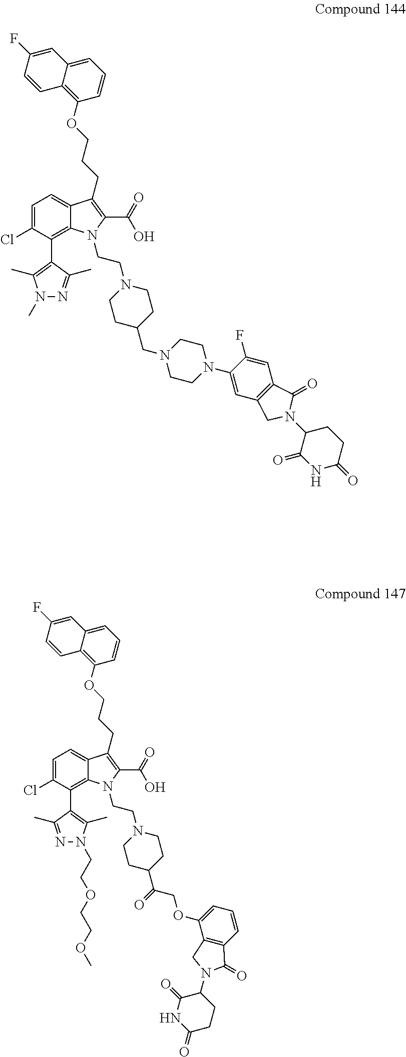 [SMILES: Cc1nn(C)c(C)c1-c1c(Cl)ccc2c(CCCOc3cccc4cc(F)ccc34)c(C(=O)O)n(CCN3CCC(CN4CCN(c5cc6c(cc5F)C(=O)N(C5CCC(=O)NC5=O)C6)CC4)CC3)c12 ; COCCOCCn1nc(C)c(-c2c(Cl)ccc3c(CCCOc4cccc5cc(F)ccc45)c(C(=O)O)n(CCN4CCC(C(=O)COc5cccc6c5CN(C5CCC(=O)NC5=O)C6=O)CC4)c23)c1C]

In accordance with a second aspect of the invention, there is provided a compound of formula (I)

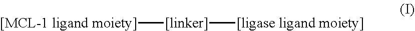

or a salt, solvate, hydrate, isomer or prodrug thereof,
wherein [ligase ligand moiety] is:
   (a) Formula (IV) or (IVa)

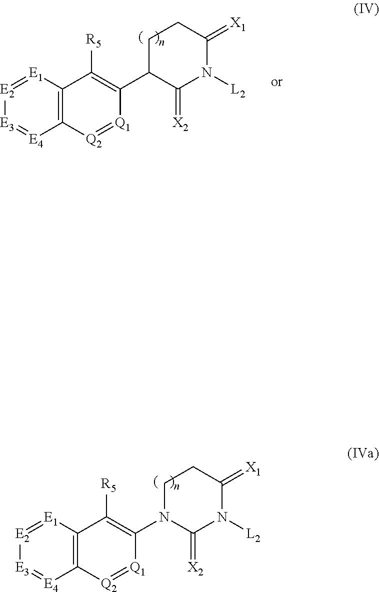

wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
   each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
   and
   each R^{b} is independently hydrogen, alkyl, alkenyl, aryl, heteroaryl, or benzyl; wherein R²¹ is a bond connected to R¹⁸ of the linker, and wherein Formula (IV) contains a single R²¹;
   (b) Formula (VIIa), (VIIb), (VIIc) or (VIId):

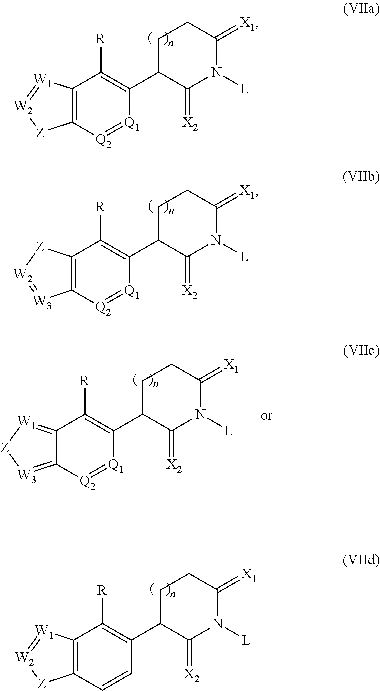

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR^{e};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII):

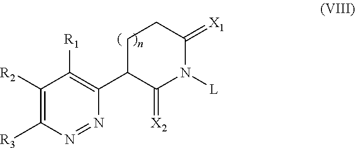

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX):

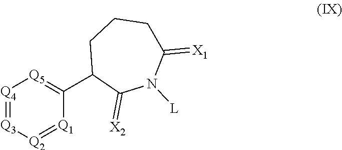

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand moiety] is a compound of Formula (A1), (A2), (A3) or (A4):

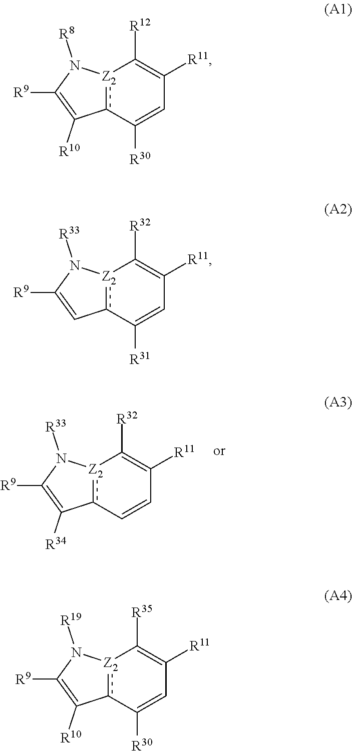

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or —P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₆ alkyl-O—R¹³, —O—C₂₋₆ alkyl-R¹³ or —C₂₋₆ alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,

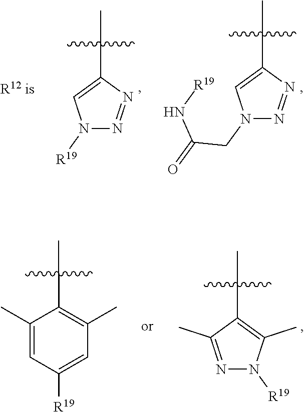

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

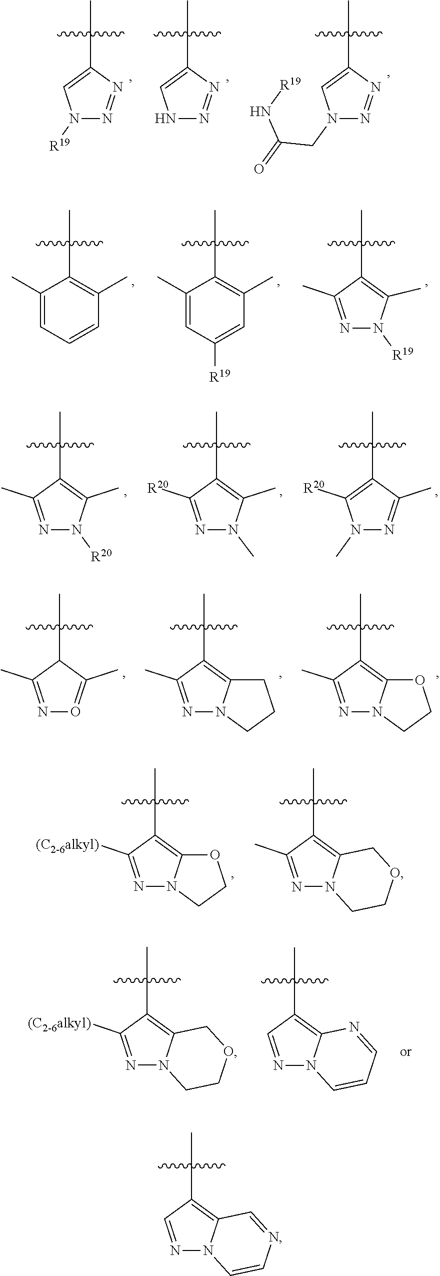

wherein R²⁰ is Me, —CH₂—OMe, —(CH₂CH₂O)ₚ(C₁-C₆ alkyl), —CH₂—O-bromobenzaldehyde, or

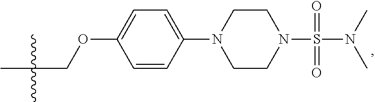

wherein p is 1-5;
R³⁴ is C₂₋₅alkyl-O—R¹³ or —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—; (C₂₋₆alky) (C₂₋₆ akyl)
R³⁵ is

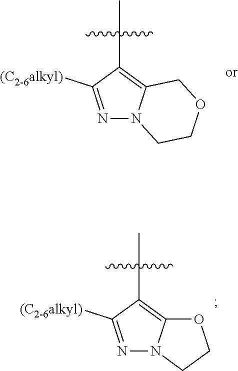

and
R¹⁹ is a bond connected to R¹⁴ of the linker, wherein each of Formula (A1), Formula (A2), Formula (A3) and Formula (A4) contains a single R¹⁹;
and wherein [linker] has the following formula

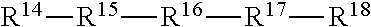

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent wherein at least one of R¹⁴-R¹⁸ is present.

In some embodiments of the second aspect:
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent; and
R¹⁸ is —C₁₋₆ alkyl, cycloalkyl, CH₂—NH—C(O)—, heterocycloalkyl, or is absent.

In some embodiments of the second aspect, [linker] is

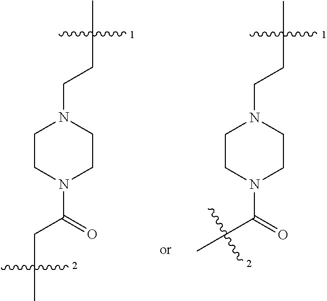 [SMILES: *CCN1CCN(C(=O)C*)CC1 ; *CCN1CCN(C(*)=O)CC1]

wherein
   [symbol] indicates attachment to [MCL-1 ligand moiety] and
   [symbol] indicates attachment to [ligase ligand moiety].

In some embodiments of the second aspect, [MCL-1 ligand moiety] is

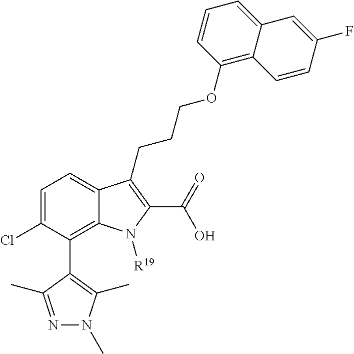

In some embodiments of the second aspect, [ligase ligand moiety] is:

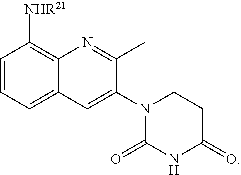

In some embodiments of the second aspect, the compound is

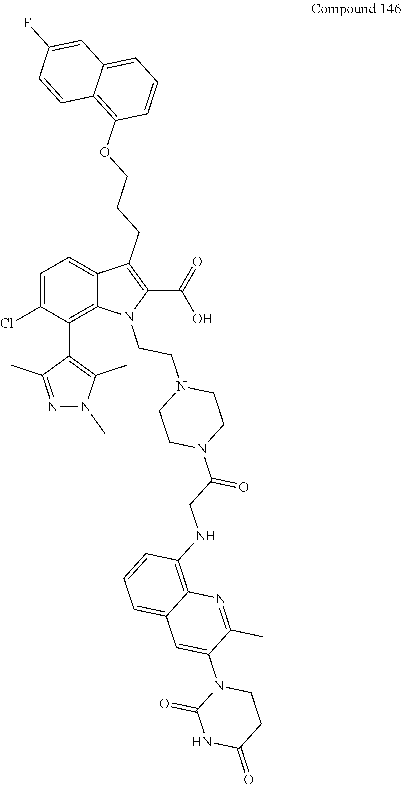

In some embodiments of the second aspect, [ligase ligand moiety] is:

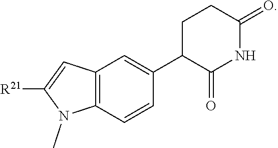

In some embodiments of the second aspect, the compound is

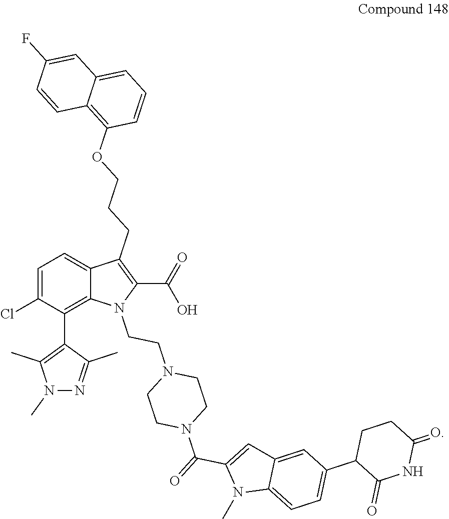 [SMILES: Cc1nn(C)c(C)c1-c1c(Cl)ccc2c(CCCOc3cccc4cc(F)ccc34)c(C(=O)O)n(CCN3CCN(C(=O)c4cc5cc(C6CCC(=O)NC6=O)ccc5n4C)CC3)c12]

In accordance with a third aspect of the invention, there is provided a compound of formula (I)

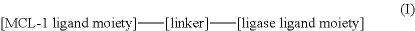

or a salt, solvate, hydrate, isomer or prodrug thereof,
wherein [ligase ligand moiety] is:
   (a) Formula (Va) or (Vb):

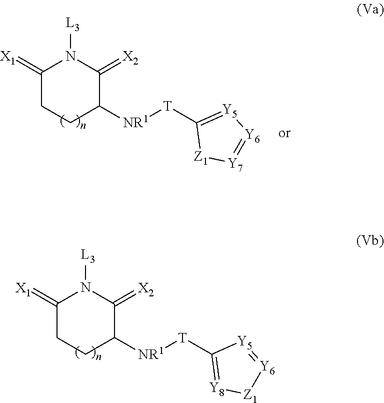

or a pharmaceutically acceptable salt or tautomer thereof,
   wherein
   each of X₁ and X₂ is independently O or S;
   Z₁ is O, S or NR⁶;
   T is is C═O or SO₂;
   R¹ is hydrogen, alkyl, cycloalkyl, alkenyl, cycloalkenyl, aryl, heteroaryl, or benzyl;
   each of Y₅, Y₆, Y₇, and Y₈ is independently N or CR⁷,
   wherein at least one of Y₅, Y₆ and Y₇ in Formula (Va) is CR⁷, and at least one of Y₅, Y₅ and Y₉ in Formula (Vb) is CR⁷;
   n is 0, 1 or 2;
   L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
   each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹; each R″″ is independently hydrogen, alkyl, cycloalkyl, alkenyl, cycloalkenyl, aryl, heteroaryl, or benzyl;
   R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
   wherein R²¹ is a bond connected to R¹⁸ of the linker, and wherein formula (Va) and formula (Vb) each contain a single R²¹;
   wherein when Z₁ is O, then Y₆ is CR⁷ and
wherein when the compound is of Formula (Va), then
   (i) when each of Y₅, Y₆ and Y₇ is CR⁷, then at least one of R⁷ is not H;
   (ii) when Z₁ is NR⁶, then Y₆ and Y₇ are CR⁷;
   (iii) when Z₁ is S, then Y₆ is not C—OMe and Y₆ is not C—OMe;
   (iv) when Z₁ is S and Y₆ is C—NHCOMe, then Y₇ is not C—CH₂NR″″C(O)OR″″;
   (v) when Z₁ is S and Y₅ is N, then Y₆ is not C—H, C-aryl or C—C(O)OR″″; and
   (vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₈ is C—H;
and when the compound is of Formula (Vb), then:
   (vii) when each of Y₅, Y₆ and Y₈ is CR⁷, then at least one of R⁷ is not H;
   (viii) when Z₁ is S, then Y₆ is not C—COOH or C—NHC(O)Me, and Y₈ is not C—Br;
   (ix) when Z₁ is S and Y₆ is C—Br, then Y₈ is C—OR″″
   (x) when Z₁ is S, Y₅ is N and Y₆ is C—H or C—NH₂, then Y₈ is not C—H
   (xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
or
   (b) Formula (IIa) or (IIb):

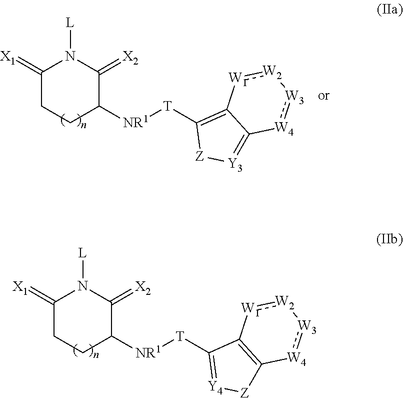

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR^{a}, wherein at least one of W₁, W₂, W₃ and W₄ is N, and
   when each [symbol] is a single bond, W₁, W₂, W₃ and W₄ are each CR^{a}₂ and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 ligand moiety] is a compound of Formula (A1), (A2), (A3) or (A4):

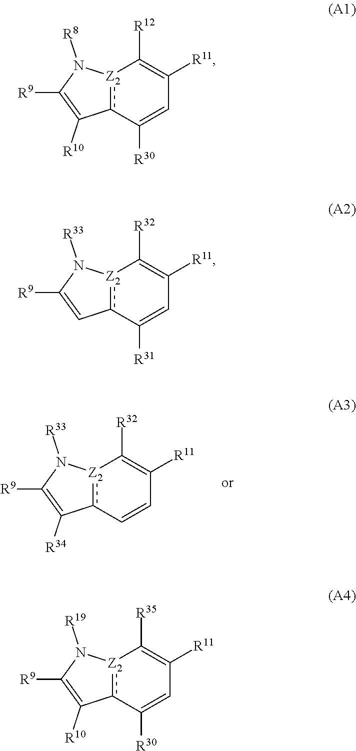

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or —PO(H₂
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,

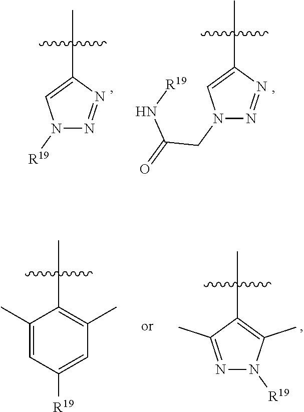

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

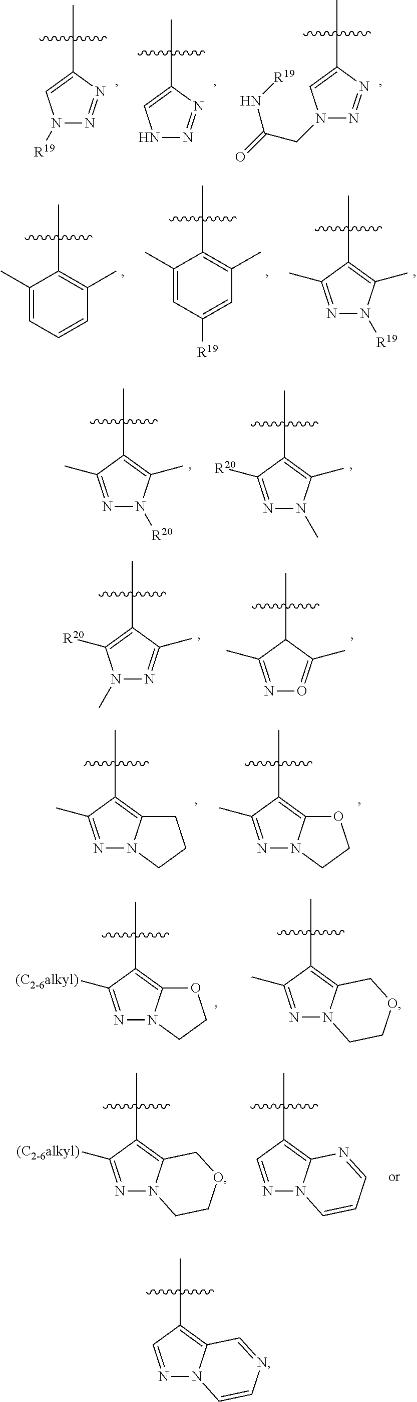

wherein R²⁰ is Me, —CH₂—OMe, —(CH₂CH₂O)ₚ(C₁-C₆ alkyl), —CH₂—O-bromobenzaldehyde, or

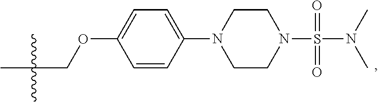

wherein p is 1-5;
R³⁴ is C₂-alkyl-O—R¹³ or —O—C₂₋₆ alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

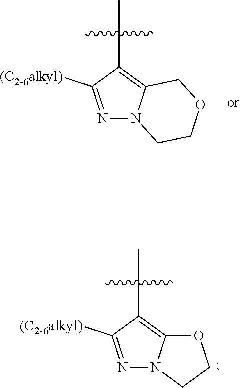

R¹⁹ is a bond connected to R¹⁴ of the linker, wherein each of Formula (A1), Formula (A2), Formula (A3) and Formula (A4) contains a single R¹⁹.
and wherein [linker] has the following formula

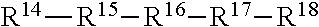

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent wherein at least one of R¹⁴-R¹⁸ is present.

In accordance with a fourth aspect of the invention, there is provided a compound of formula (I)

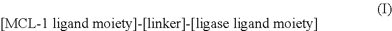

or a salt, solvate, hydrate, isomer or prodrug thereof,
wherein [ligase ligand moiety] is:
   (a) Formula (VIa) or (VIb):

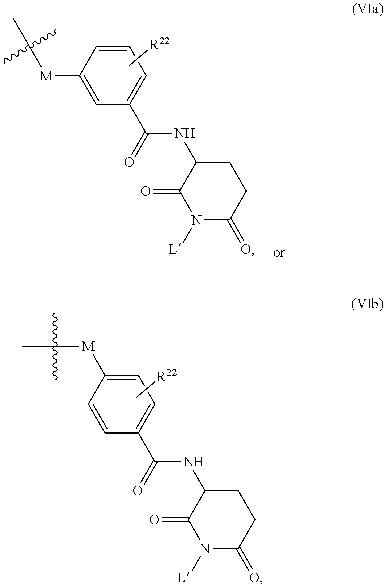

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl
   (b) Formula (II):

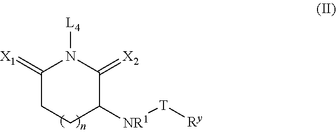

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from

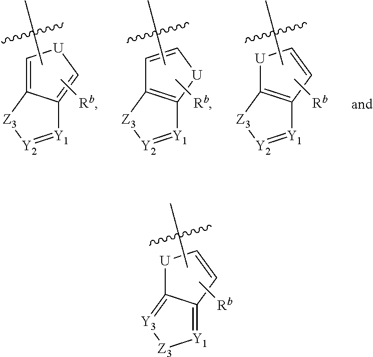

wherein [symbol] indicates attachment to T,
   Z₃ is O, S or NR³;
   U is O, S, NR^{b} or CRⁱ₂;
   each of Y₁, Y₂ and Y₃ is independently N or CR^{d};
   each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
   each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
   each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
   each R^{b} is independently alkyl, cycloalkyl, alkenyl, cycloalkenyl, aryl, heteroaryl, or benzyl;
   R²¹ is a bond connected to R¹⁸ of the linker, wherein Formula (II) contains a single R²¹;
   wherein,
   (i) when R^{y} is

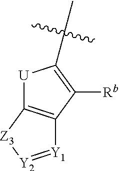

then Y₂ is CR^{d}; and
(ii) when R^{y} is

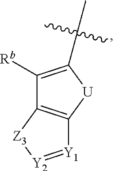

then Rⁱ in CRⁱ₂ is not hydrogen or
(c) Formula (III):

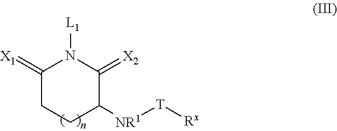

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from

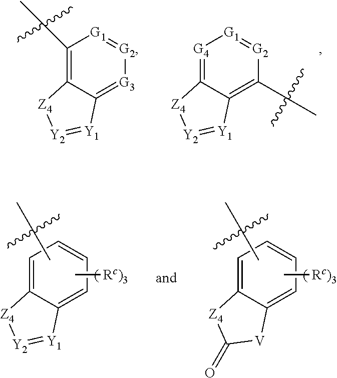

wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{C}, then C=X₁ may be replaced by CH;
and wherein:
   (i) when R^{x} is

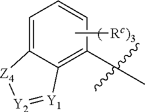

and Z₄ is NH, then L₁ is hydrogen, —CH₂C(O)OR^{b}, or —OR^{b};
(ii) when R^{x} is

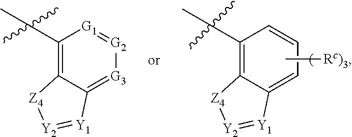

Z₄ is NR⁴, Y₁ is CR^{f}, and Y₂ is N, then R⁴ is not alkyl and at least one of R² and R is not H;
(iii) when R^{x} is

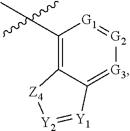

Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not

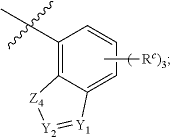

(v) when R^{x} is

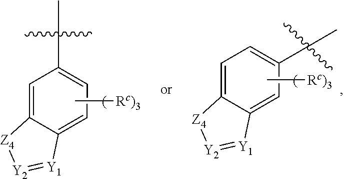

Z₄ is NR⁴, and Y₁ or Y₂ is N, then R⁴ is not alkyl;
(vi) when R^{x} is

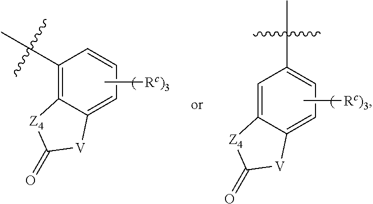

then n=1 or 2; and
(vii) when R^{x} is

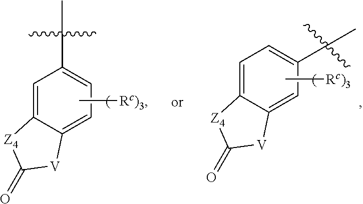

then Z₄=O or S
wherein [MCL-1 ligand moiety] is a compound of Formula (A1), (A2), (A3) or (A4):

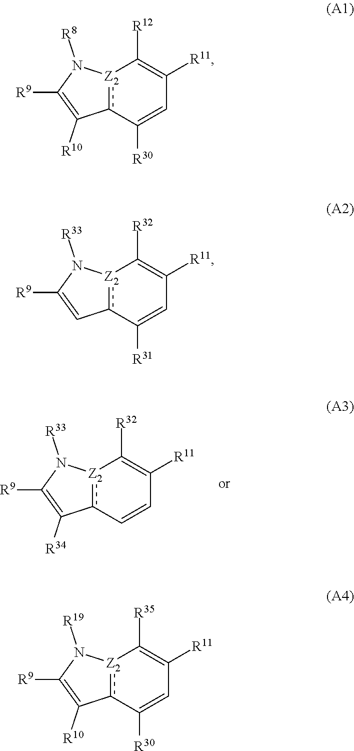

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or —P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,

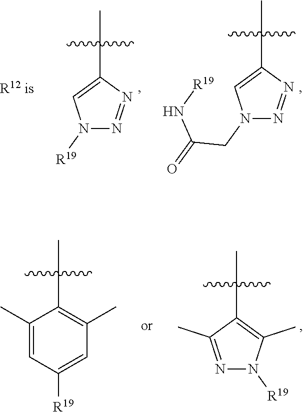

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

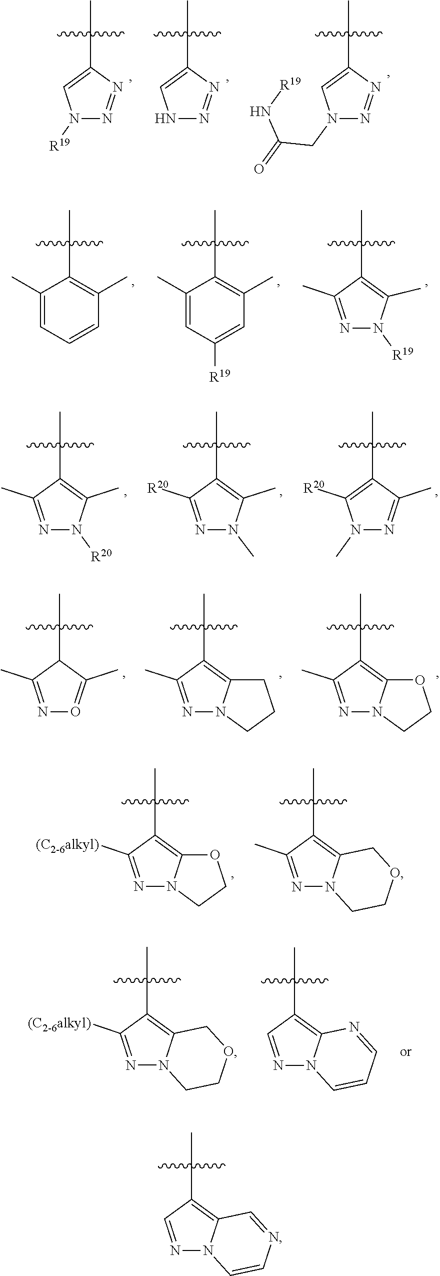

wherein R²⁰ is Me, —CH₂—OMe, —(CH₂CH₂O)P(C₁-C₆ alkyl), —CH₂—O-bromobenzaldehyde, or

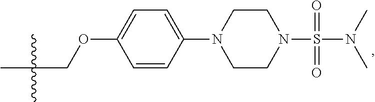

wherein p is 1-5;
R³⁴ is C₂₋₅alkyl-O—R¹³ or —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

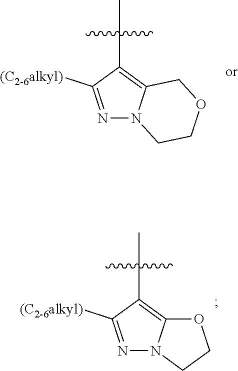

and
R¹⁹ is a bond connected to R¹⁴ of the linker, wherein each of Formula (A1), Formula (A2), Formula (A3) and Formula (A4) contains a single R¹⁹;
and wherein [linker] has the following formula

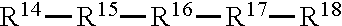

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent
   wherein at least one of R¹⁴-R¹⁸ is present.

In some embodiments of the fourth aspect, [ligase ligand moiety] is

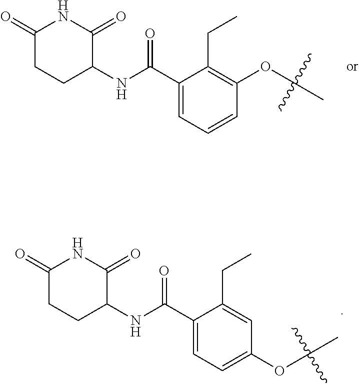 [SMILES: *Oc1cccc(C(=O)NC2CCC(=O)NC2=O)c1CC ; *Oc1ccc(C(=O)NC2CCC(=O)NC2=O)c(CC)c1]

In some embodiments of the fourth aspect,
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent; and
R¹⁸ is —C₁₋₆ alkyl, cycloalkyl, CH₂—NH—C(O)—, heterocycloalkyl, or is absent.

In some embodiments of the fourth aspect, [linker] is

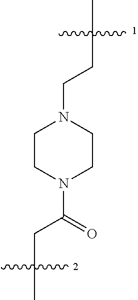 [SMILES: *CCN1CCN(C(=O)C*)CC1]

wherein
   [symbol] indicates attachment to [MCL-1 ligand moiety] and
[symbol] indicates attachment to [ligase ligand moiety].

In some embodiments of the fourth aspect, [MCL-1 ligand moiety] is

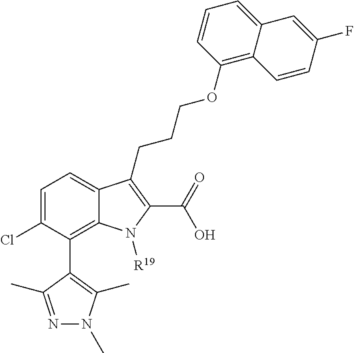

In some embodiments of the fourth aspect, the compound is

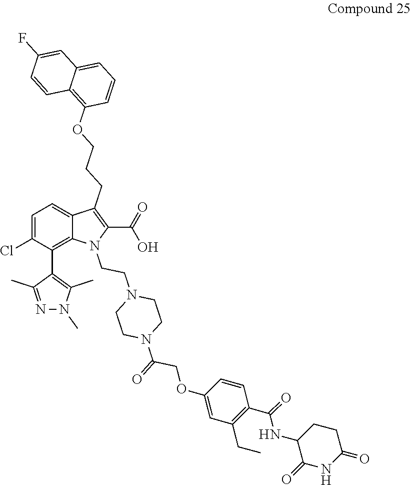 [SMILES: CCc1cc(OCC(=O)N2CCN(CCn3c(C(=O)O)c(CCCOc4cccc5cc(F)ccc45)c4ccc(Cl)c(-c5c(C)nn(C)c5C)c43)CC2)ccc1C(=O)NC1CCC(=O)NC1=O]

In some embodiments of the fourth aspect, [ligase ligand moiety] is Formula (III):

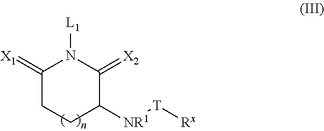

In some embodiments of the fourth aspect, [ligase ligand moiety] is

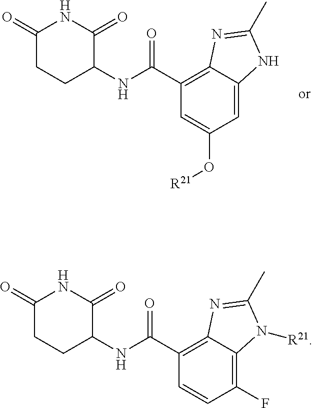

In accordance with a fifth aspect of the invention, there is provided a compound of formula (I)

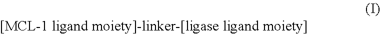

or a salt, solvate, hydrate, isomer or prodrug thereof,
wherein [ligase ligand moiety] is:

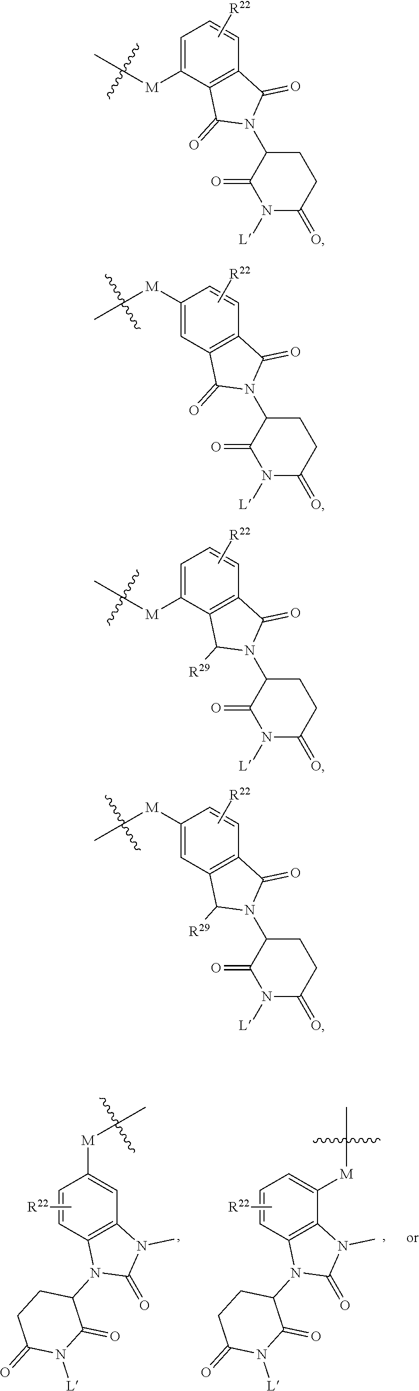

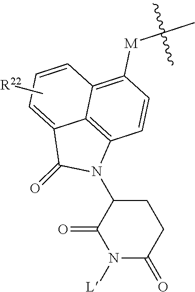

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   R²⁹ is hydrogen or Me
   L′ is hydrogen, alkyl, benzyl, acetyl or pivaloyl;
wherein [MCL-1 ligand moiety] is a compound of Formula (A):

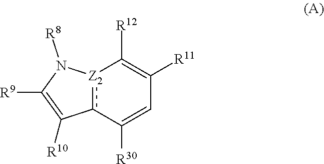

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

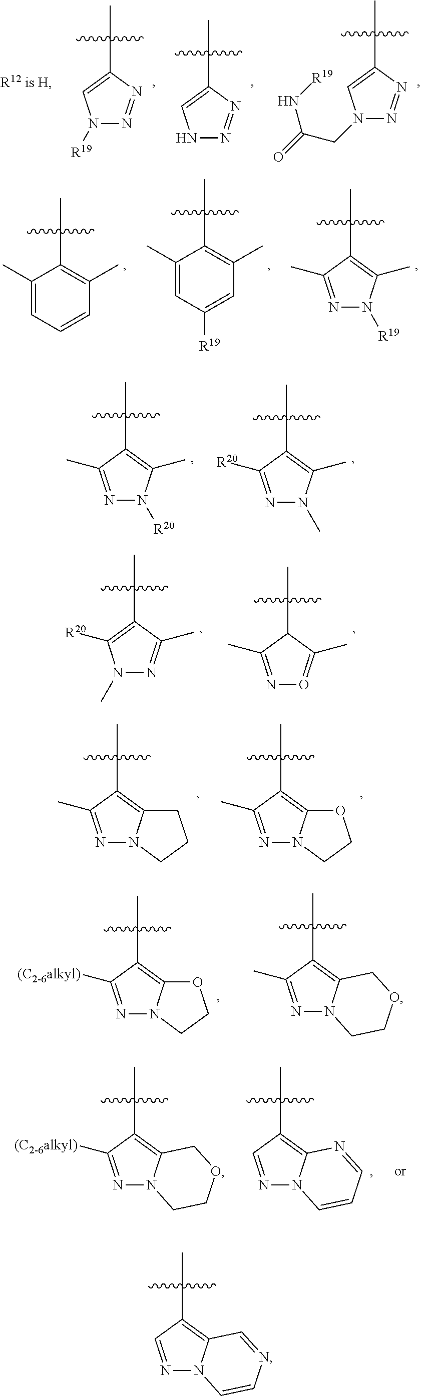

wherein R²⁰ is Me, —CH₂—OMe, —(CH₂CH₂O)ₚ(C₁-C₆ alkyl), or —CH₂—O-bromobenzaldehyde, wherein p is 1-5;
or when R¹² is

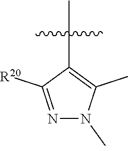

and R¹⁰ is —O-naphthyl substituted with —O— or —S—, then R²⁰ is

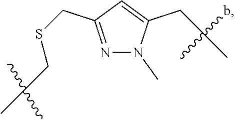

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

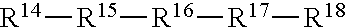

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl-C(O)—, cycloalkyl or —CH₂—NH—C(O)—.

In some embodiments of the fifth aspect, [ligase ligand moiety] is

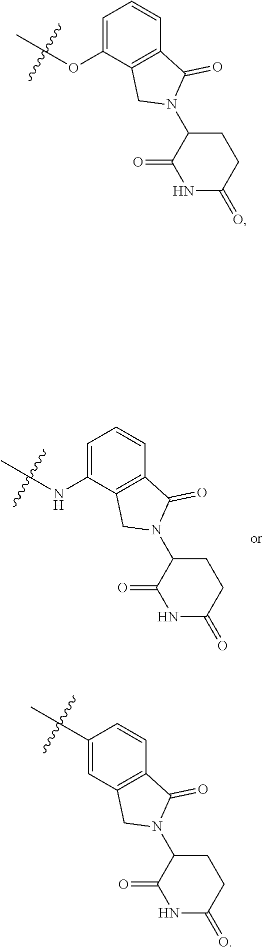 [SMILES: *Oc1cccc2c1CN(C1CCC(=O)NC1=O)C2=O ; *Nc1cccc2c1CN(C1CCC(=O)NC1=O)C2=O ; *c1ccc2c(c1)CN(C1CCC(=O)NC1=O)C2=O]

In some embodiments of the fifth aspect,
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
R¹⁸ is cycloalkyl or —CH₂—NH—C(O)—.

In some embodiments of the fifth aspect, R¹⁴ is —C₁₋₆ alkyl, —C₂₋₆ alkenyl, —C₂₋₆ alkynyl, —C(O)—, —SO₂— or is absent. In some embodiments, R¹⁴ is —C₁₋₆ alkyl.

In some embodiments of the fifth aspect, R¹⁵ is heterocycloalkyl. In some such embodiments, R¹⁵ is piperazine,

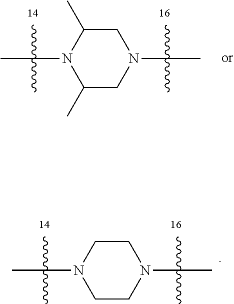

wherein [symbol] indicates attachment to R¹⁴ and [symbol] indicates attachment to R¹⁶.

In some embodiments of the fifth aspect, R¹⁶ is —C₁₋₆ alkyl, —CH₂—C(O)— or —C(O)—.

In some embodiments of the fifth aspect, R¹⁷ is absent.

In some embodiments of the fifth aspect, R¹⁸ is cyclobutyl.

In some embodiments of the fifth aspect, [MCL-1 ligand moiety] is

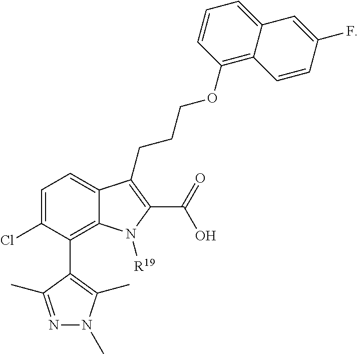

In accordance with a sixth aspect of the invention, there is provided a compound of formula (I)

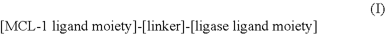

or a salt, solvate, hydrate, isomer or prodrug thereof,
wherein [ligase ligand moiety] is:
   (a) Formula (IV) or (IVa)

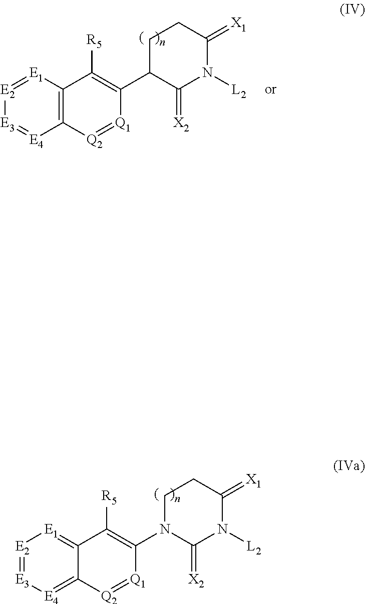

wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
and
each R^{b} is independently hydrogen, alkyl, alkenyl, aryl, heteroaryl, or benzyl;
wherein R²¹ is a bond connected to R¹⁸ of the linker, and wherein Formula (IV) contains a single R²¹;
   (b) Formula (VIIa), (VIIb), (VIIc) or (VIId):

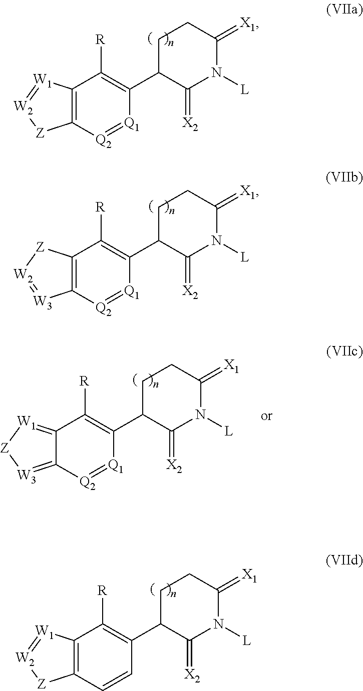

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR^{e};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII):

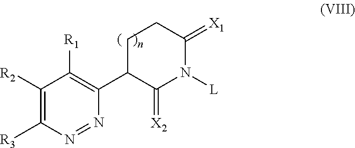

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX):

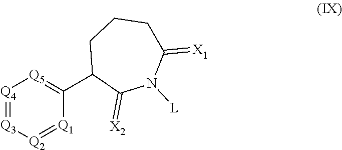

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand moiety] is a compound of Formula (A):

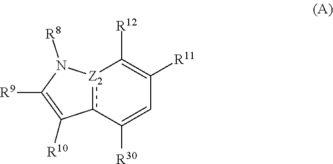

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

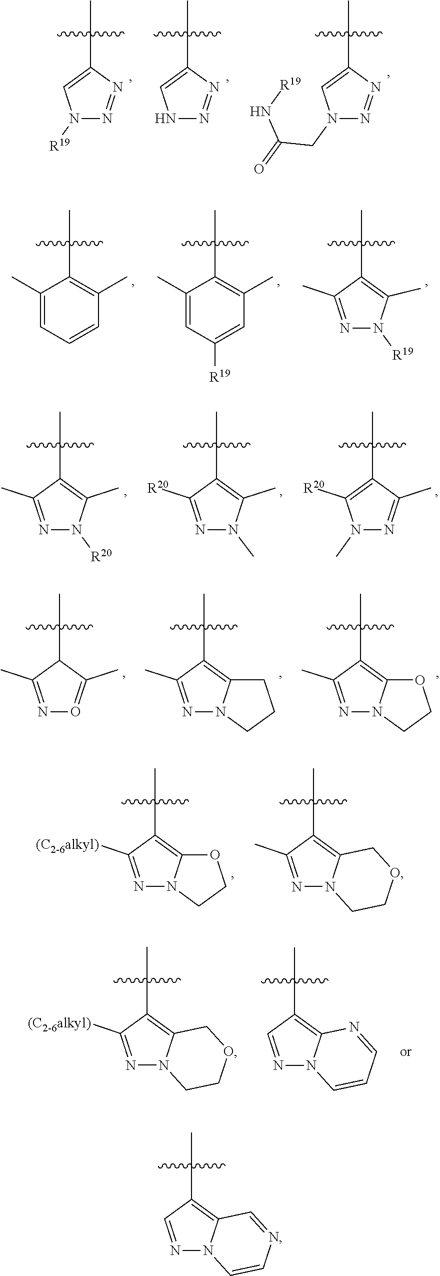

wherein R²¹ is Me, —CH₂—OMe, —(CH₂CH₂O)ₚ(C₁-C₆ alkyl), —CH₂—O-bromobenzaldehyde, or

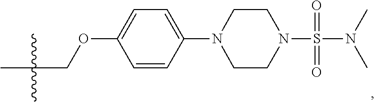

wherein p is 1-5;
or when R¹² is

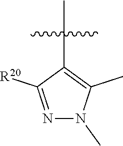

and R¹⁰ is —O-naphthyl substituted with —O— or —S—, then R²⁰ is

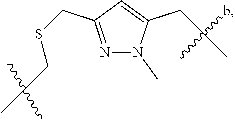

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

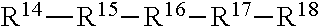

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl-C(O)—, cycloalkyl or —CH₂—NH—C(O)—.

In some embodiments of the sixth aspect, [ligase ligand moiety] is Formula (IV):

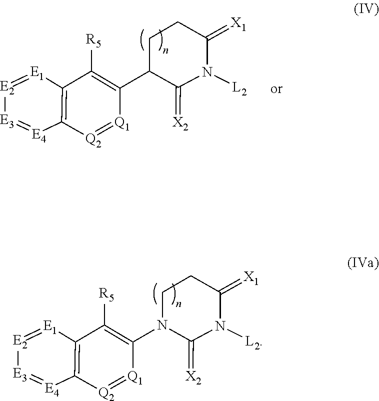

In some embodiments of the sixth aspect, each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹.

In some embodiments of the sixth aspect, each R^{b} is independently hydrogen, alkyl, cycloalkyl, or aryl.

In some embodiments of the sixth aspect, the aryl is substituted with one or more groups selected from halogen, alkyl and O-haloalkyl, optionally wherein the halogen is Cl, the alkyl is methyl and the O-haloalkyl is O—CF₃.

In some embodiments of the sixth aspect, one of E₁, E₂, E₃ and E₄ is N, and the remaining three of E₁, E₂, E₃ and E₄ are each CR′. In some embodiments, E₁ is N, and E₂, E₃ and E₄ are CR′. In other embodiments, E₂ is N, and E₁, E₃ and E₄ are CR′. In other embodiments, E₃ is N, and E₁, E₂ and E₄ are CR′. In other embodiments, E₄ is N, and E₁, E₂ and E₃ are CR′.

In some embodiments of the sixth aspect, E₁, E₂, E₃ and E₄ are each CR′, optionally wherein E₁, E₂, E₃ and E₄ are each CH.

In some embodiments of the sixth aspect, three of E₁, E₂, E₃ and E₄ are CH, and one of E₁, E₂, E₃ and E₄ is C-halogen, C-alkyl, C-alkenyl, C-alkynyl, C-aryl, C-heteroaryl, C-benzyl, C-haloalkyl, C-haloalkenyl, C—NH₂, C—NHR^{b}, C—NR^{b}₂, C—NR^{b}C(O)R^{b}, C—NR^{b}C(O)OR^{b}, C—NO₂, C—CN, C—C(O)R^{b}, C—C(O)OR^{b}, C—C(O)NH₂, C—C(O)NHR^{b}, C—C(O)NR^{b}₂, C—C(O)NHCHR^{b}₂, C—CHR^{b}NHC(O)NHR^{b}, C—CHR^{b}NHC(O)C(halogen)₂R^{b}, C—OR^{b}, C—OC(O)R^{b}, C—OC(O)OR^{b}, C—OC(O)NH₂, C—OC(O)NHR^{b}, C—OC(O)NR^{b}₂, C—SR^{b}, C—S(O)₂R^{b}, C—S(O)₂OR^{b}, C—S(O)₂NH₂, C—S(O)₂NHR^{b}, C—S(O)₂NR^{b}₂, C—NHS(O)₂R^{b}, —R²¹, —O—R²¹, —NH—R²¹, —C(O)—R²¹, —C(O)—NH—R²¹, or —CH₂—NH—C(O)—R²¹. In some such embodiments, E₂, E₃ and E₄ are each CH. In other embodiments, E₁, E₃ and E₄ are each CH. In other embodiments, E₁, E₂ and E₄ are each CH.

In some embodiments of the sixth aspect, two of E₁, E₂, E₃ and E₄ are N, and the remaining two of E₁, E₂, E₃ and E₄ are each CR′.

In some embodiments of the sixth aspect, three of E₁, E₂, E₃ and E₄ are N, and the remaining one of E₁, E₂, E₃ and E₄ is CR′.

In some embodiments of the sixth aspect, Q₁ is N and Q₂ is CR. In other embodiments, Q₁ is N and Q₂ is N. In other embodiments, Q₁ is CR and Q₂ is N. In some such embodiments, Q₁ is C—H or C-alkyl. In some such embodiments, Q₁ is C—H or C-Me.

In some embodiments of the sixth aspect,
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent;
R¹⁷ is —CH₂(C₂H₄—O)_{y}, (C₂H₄—O)ₓ, (C₃H₆—O)ₓ, or is absent; and
R¹⁸ is cycloalkyl or —CH₂—NH—C(O)—.

In some embodiments of the sixth aspect, R¹⁴ is —C₁₋₆ alkyl, —C₂₋₆ alkenyl, —C₂₋₆ alkynyl, —C(O)—, —SO₂— or is absent. In some embodiments, R¹⁴ is —C₁₋₆ alkyl.

In some embodiments of the sixth aspect, R⁵ is cycloalkyl, heterocycloalkyl, aryl, heteroaryl, —C₁₋₆ alkyl-NH—, -cycloalkyl-NH— or is absent. In some embodiments, R⁵ is heterocycloalkyl or is absent. In some embodiments, R⁵ is piperazine.

In some embodiments of the sixth aspect, R¹⁶ is —C(O)—.

In some embodiments of the sixth aspect, R¹⁷ is absent.

In some embodiments of the sixth aspect, R¹⁸ is —CH₂—NH—C(O)—.

In some embodiments of the sixth aspect, [linker] is

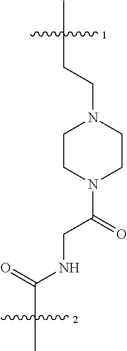 [SMILES: *CCN1CCN(C(=O)CNC(*)=O)CC1]

wherein
   [symbol] indicates attachment to [MCL-1 ligand moiety] and
   [symbol] indicates attachment to [ligase ligand moiety].

In some embodiments of the sixth aspect, [MCL-1 ligand moiety] is:

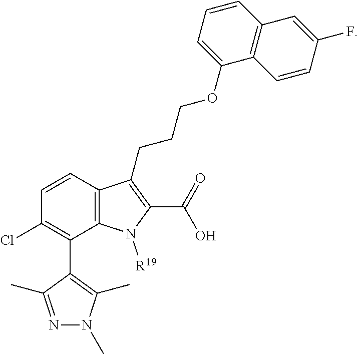

In some embodiments of the sixth aspect, the compound is selected from:

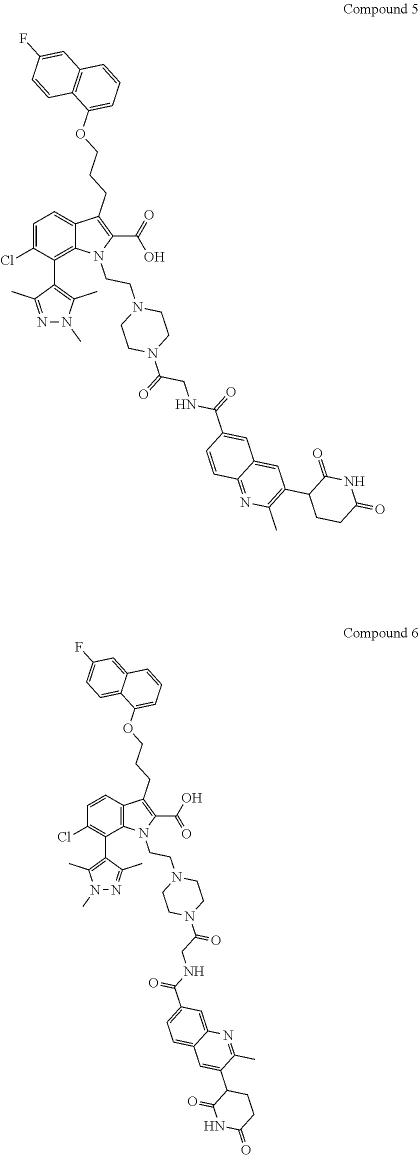

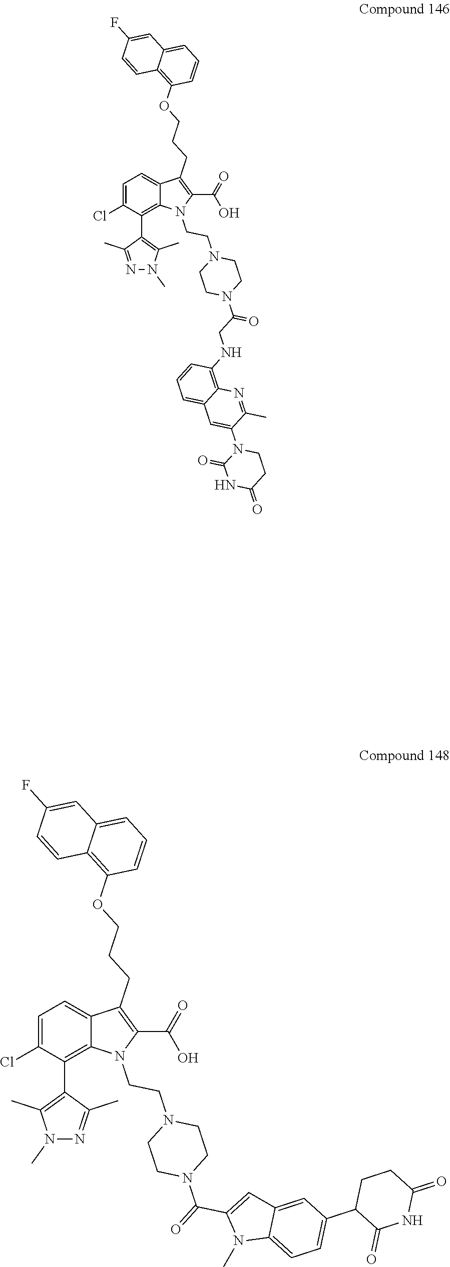

In some embodiments, the compound is selected from Compounds 6, 146 and 148.

In some embodiments of the sixth aspect, the compound is

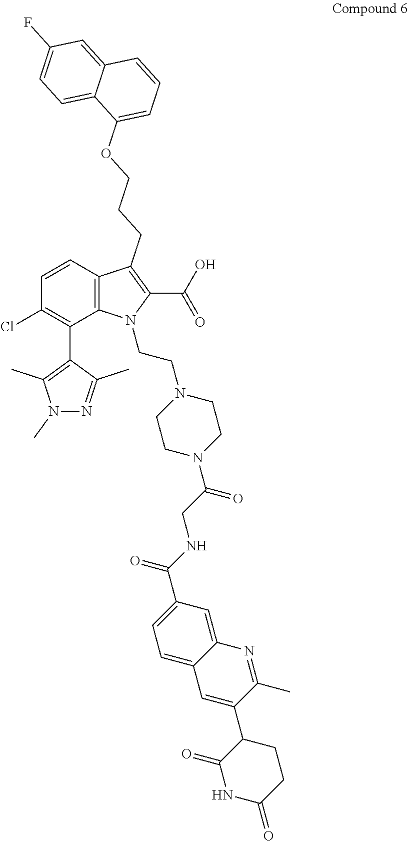

In accordance with a seventh aspect of the invention, there is provided a compound of formula (I)

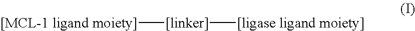

or a salt, solvate, hydrate, isomer or prodrug thereof,
wherein [ligase ligand moiety] is:
   (a) Formula (Va) or (Vb):

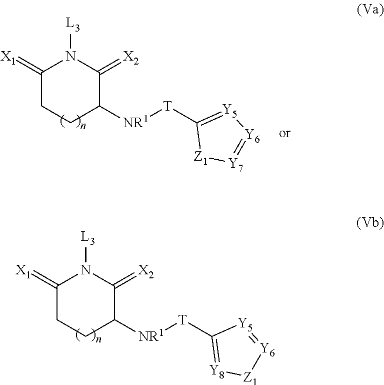

or a pharmaceutically acceptable salt or tautomer thereof,
   wherein
   each of X₁ and X₂ is independently O or S;
   Z₁ is O, S or NR⁶;
   T is is C═O or SO₂;
   R¹ is hydrogen, alkyl, cycloalkyl, alkenyl, cycloalkenyl, aryl, heteroaryl, or benzyl;
   each of Y₅, Y₆, Y₇, and Y₈ is independently N or CR⁷,
   wherein at least one of Y₅, Y₆ and Y₇ in Formula (Va) is CR⁷, and at least one of Y₅, Y₅ and Y₈ in Formula (Vb) is CR⁷;
   n is 0, 1 or 2;
   L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
   each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
   each R″″ is independently hydrogen, alkyl, cycloalkyl, alkenyl, cycloalkenyl, aryl, heteroaryl, or benzyl;
   R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
   wherein R²¹ is a bond connected to R¹⁸ of the linker, and wherein formula (Va) and formula (Vb) each contain a single R²¹;
   wherein when Z₁ is O, then Y₆ is CR⁷ and
wherein when the compound is of Formula (Va), then
   (i) when each of Y₅, Y₆ and Y₇ is CR⁷, then at least one of R⁷ is not H;
   (ii) when Z₁ is NR⁶, then Y₆ and Y₇ are CR⁷;
   (iii) when Z₁ is S, then Y₅ is not C—Ome and Y₆ is not C—Ome;
   (iv) when Z₁ is S and Y₅ is C—NHCOMe, then Y₇ is not C—CH₂NR″″C(O)OR″″;
   (v) when Z₁ is S and Y₅ is N, then Y₆ is not C—H, C-aryl or C—C(O)OR″″; and
   (vi) when Z₁ is S and Y₅ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
   and when the compound is of Formula (Vb), then:
   (vii) when each of Y₅, Y₆ and Y₈ is CR⁷, then at least one of R⁷ is not H;
   (viii) when Z₁ is S, then Y₅ is not C—COOH or C—NHC(O)Me, and Y₈ is not C—Br;
   (ix) when Z₁ is S and Y₆ is C—Br, then Y₈ is C—OR″″
   (x) when Z₁ is S, Y₅ is N and Y₆ is C—H or C—NH₂, then Y₈ is not C—H
   (xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y⁵, Y₆ and Y₈ are CR⁷.
Or
   (b) Formula (IIa) or (IIb):

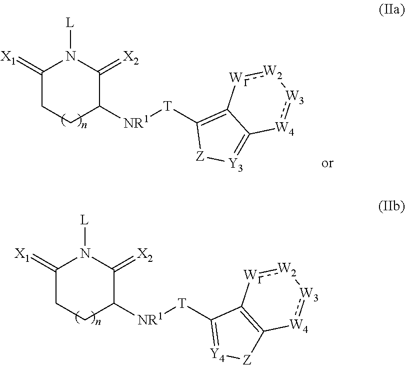

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR^{a}, wherein at least one of W₁, W₂, W₃ and W₄ is N, and
   when each [symbol] is a single bond, W₁, W₂, W₃ and W₄ are each CR^{a}₂ and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
   each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
   each R^{h} is independently hydrogen, alkyl, cycloalkyl, heterocycloalkyl, alkenyl, cycloalkenyl, aryl, heteroaryl, or benzyl;
   R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
   R¹ is hydrogen, alkyl, cycloalkyl, alkenyl, cycloalkenyl, aryl, heteroaryl, or benzyl;
   R²¹ is a bond connected to R¹⁸ of the linker, and wherein formula (IIa) and formula (IIb) each contain a single R²¹;
   wherein when each [symbol] is a double bond, Z is NR², R² is hydrogen, and each R^{a} is hydrogen, then W₄ is CR^{a};
wherein [MCL-1 ligand moiety] is a compound of Formula (A):

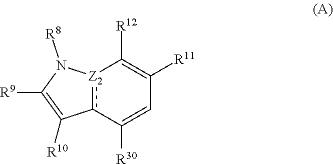

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C—C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,
   R¹² is Hm

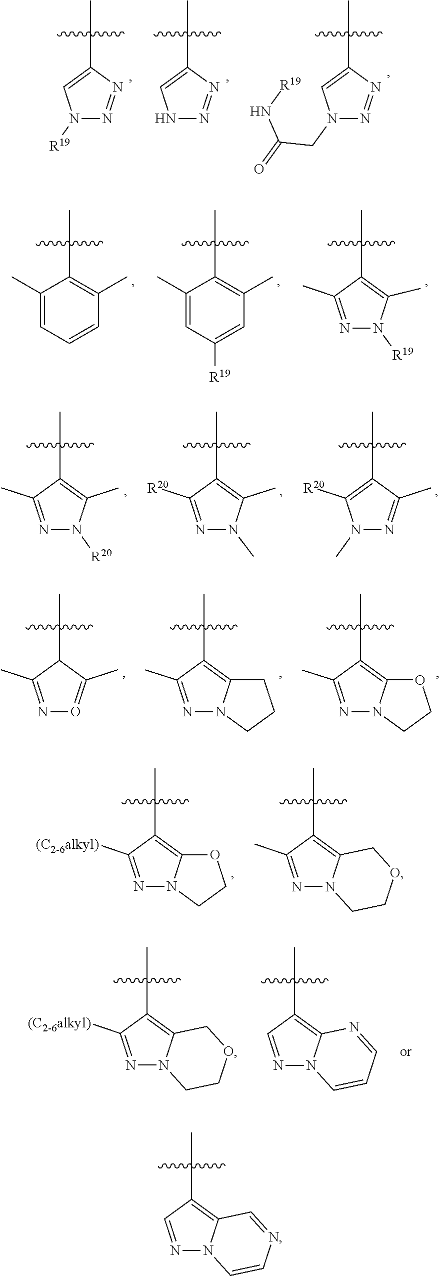

wherein R²¹ is Me, CH₂—OMe, (CH₂CH₂O)ₚ(C₁-C₆ alkyl), —CH₂—O-bromobenzaldehyde, or

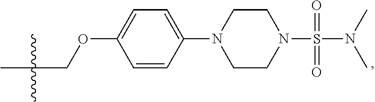

wherein p is 1-5;
or when R¹² is

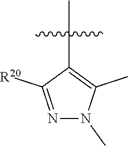

and R¹⁰ is —O-naphthyl substituted with —O— or —S—, then R²⁰ is

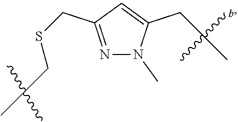 [SMILES: *CSCc1cc(C*)n(C)n1]

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

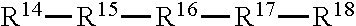

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl-C(O)—, cycloalkyl or CH₂—NH—C(O)—.

In accordance with an eighth aspect of the invention, there is provided a compound of formula (I)

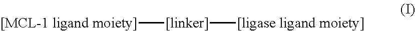

or a salt, solvate, hydrate, isomer or prodrug thereof,
wherein [ligase ligand moiety] is:
   (a) Formula (VIa) or (VIb):

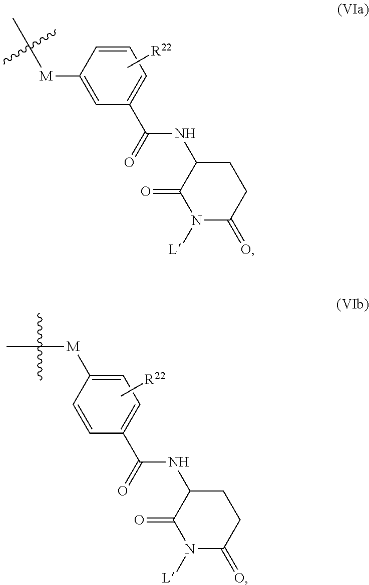

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl
   (b) Formula (II):

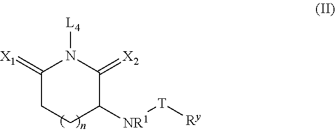

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from

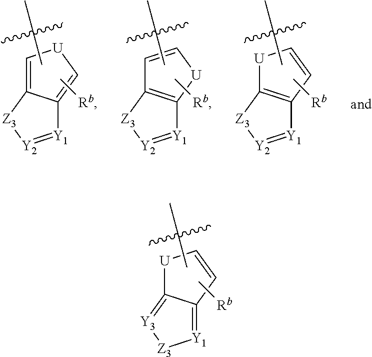

wherein [symbol] indicates attachment to T,
   Z₃ is O, S or NR³;
   U is O, S, NR^{b} or CRⁱ₂;
   each of Y₁, Y₂ and Y₃ is independently N or CR^{d};
   each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
   each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
   each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
   each R^{b} is independently alkyl, cycloalkyl, alkenyl, cycloalkenyl, aryl, heteroaryl, or benzyl;
   R²¹ is a bond connected to R¹⁸ of the linker, wherein Formula (II) contains a single R²¹;
   wherein,
   (i) when R^{y} is

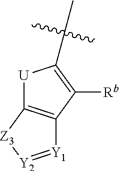

then Y₂ is CR^{d}; and
(ii) when R^{y} is

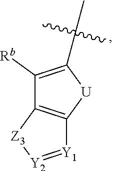

then Rⁱ in CRⁱ₂ is not hydrogen
or
   (c) Formula (III):

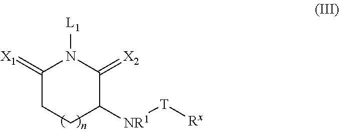

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from

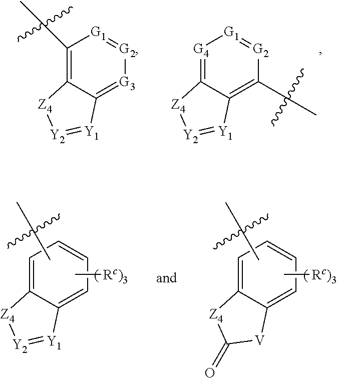

wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{C}, then C=X₁ may be replaced by CH;
and wherein:
   (i) when R^{x} is

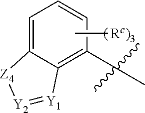

and Z₄ is NH, then L₁ is hydrogen, —CH₂C(O)OR^{b}, or —OR^{b};
(ii) when R^{x} is

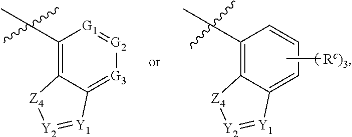

1, Z₄ is NR⁴, Y₁ is CR^{f}, and Y₂ is N, then R⁴ is not alkyl and at least one of R² and R is not H;
(iii) when R^{x} is

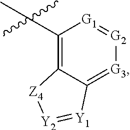

Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not

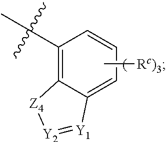

(v) when R^{x} is

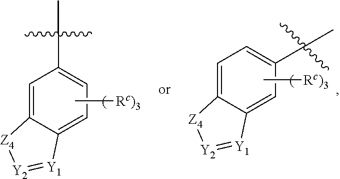

Z₄ is NR⁴, and Y₁ or Y₂ is N, then R⁴ is not alkyl;
(vi) when R^{x} is

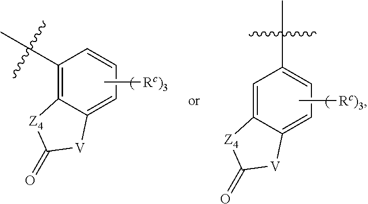

then n=1 or 2; and
(vii) when R^{x} is

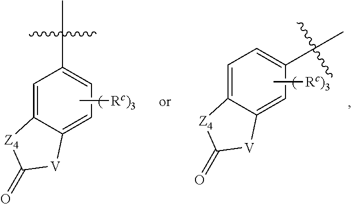

then Z₄=O or S
wherein [MCL-1 ligand moiety] is a compound of Formula (A):

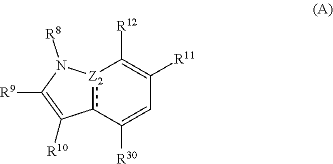

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

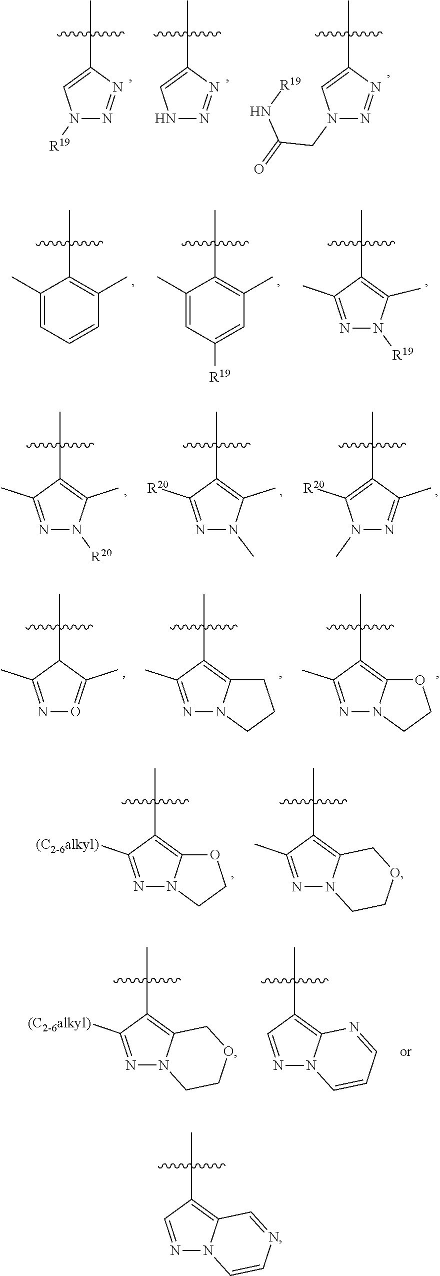

wherein R²⁰ is Me, —CH₂—OMe, —(CH₂CH₂O)ₚ(C₁-C₆ alkyl), —CH₂—O-bromobenzaldehyde, or

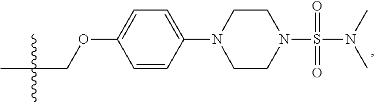

wherein p is 1-5; or
when R¹² is

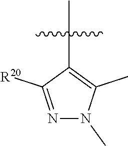

and R¹⁰ is —O-naphthyl substituted with —O— or —S—, then R²⁰ is

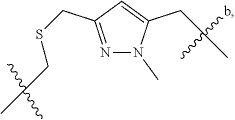

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

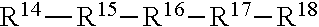

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl-C(O)—, cycloalkyl or CH₂—NH—C(O)—.

In some embodiments of the seventh and eighth aspects,
R¹⁶ is —C₁₋₆ alkyl, —C(O)—, C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent; and
R¹⁸ is cycloalkyl or CH₂—NH—C(O)—.

In some embodiments of any of the fifth to eighth aspects, one of R¹⁰ and R³⁰ is H and the other of R¹⁰ and R³⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

In some embodiments of any of the fifth to eighth aspects, R³⁰ is H.

In some embodiments of any of the fifth to eighth aspects, R¹² is H,

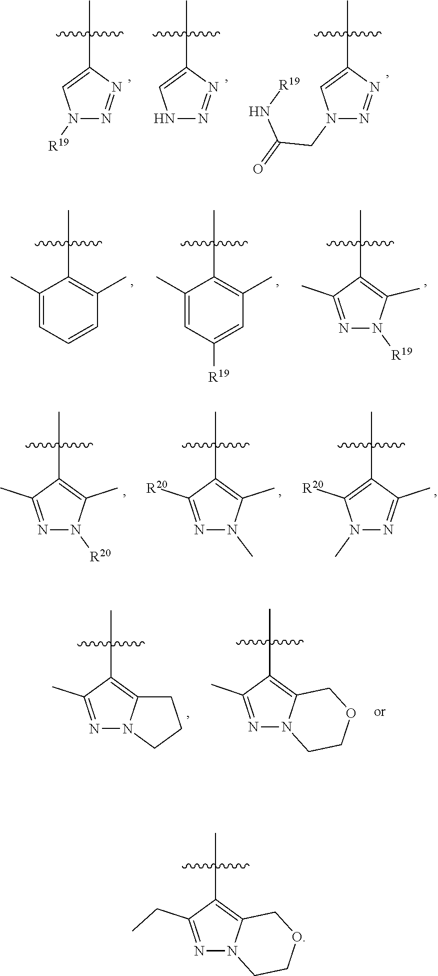

In some embodiments of any of the fifth to eighth aspects, R²⁰ is Me, —CH₂—O-bromobenzaldehyde, or

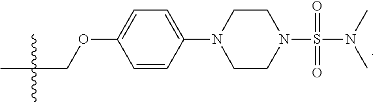 [SMILES: *COc1ccc(N2CCN(S(=O)(=O)N(C)C)CC2)cc1]

In some embodiments of any of the fifth to eighth aspects, Z₂ is C and [symbol] is a double bond.

In some embodiments of any of the fifth to eighth aspects, R¹¹ is hydrogen. In other embodiments, R¹¹ is halogen.

In some embodiments of any of the fifth to eighth aspects, [MCL-1 ligand moiety] is:

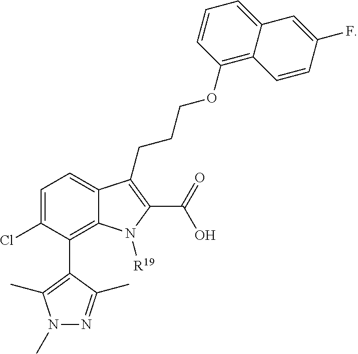

In accordance with a ninth aspect of the present invention, there is provided a compound of formula (I)

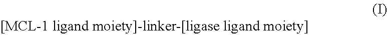

or a salt, solvate, hydrate, isomer or prodrug thereof,
wherein [ligase ligand moiety] is:

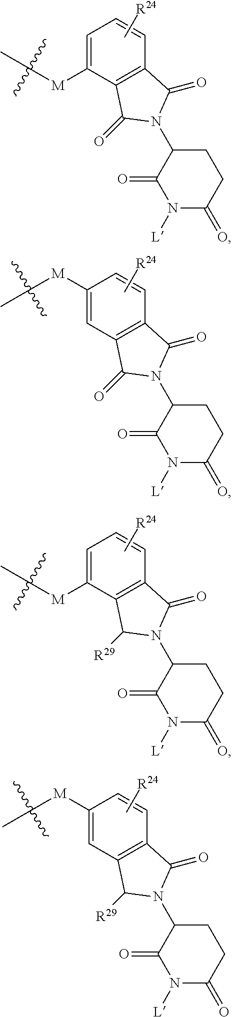

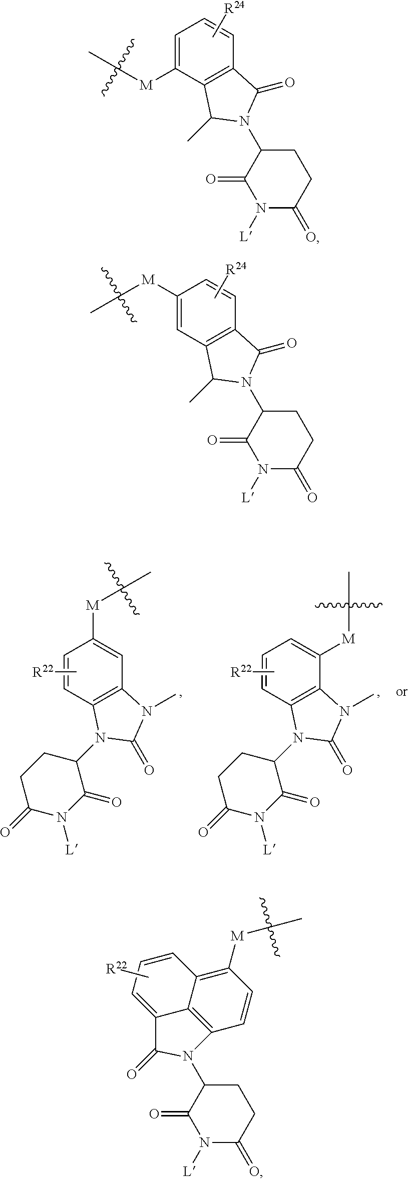

wherein
   M is O, S or NH, or is absent;
   [symbol] indicates attachment to R¹⁸ of the linker;
   R²⁴ is —OMe or heterocycloalkyl
   R²² is hydrogen, halogen, —OMe, an amino group, heterocycloalkyl, or unsubstituted C₁-C₆ alkyl;
   R²⁹ is hydrogen or Me; and
   L′ is hydrogen, alkyl, benzyl, acetyl or pivaloyl;
wherein [MCL-1 ligand moiety] is a compound of Formula (A):

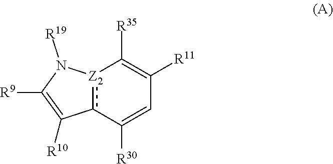

wherein
   [symbol] is a single bond or a double bond;
   R⁸ is H, R¹⁹, or C₁-C₅ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₅ alkyl,

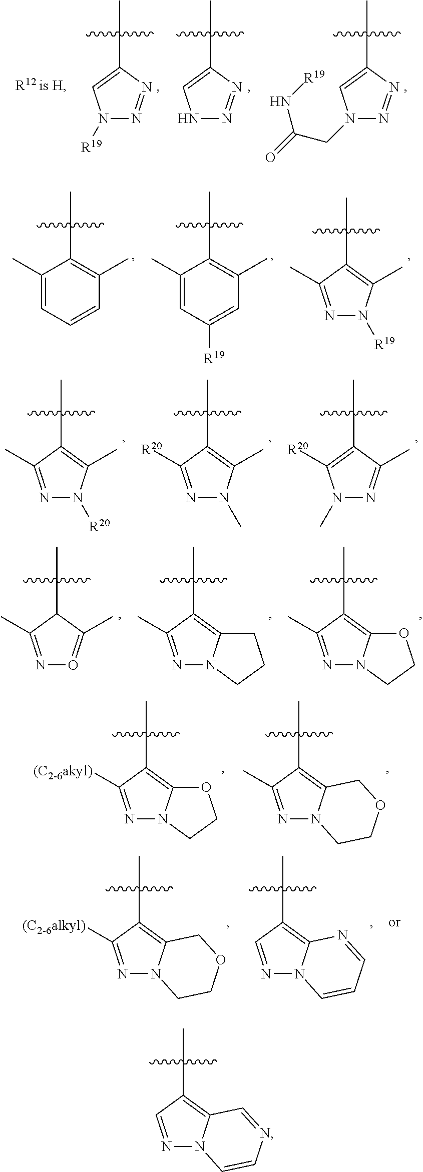

wherein R²⁰ is Me, —CH₂—OMe, —(CH₂CH₂O)ₚ(C₁-C₆ alkyl), or —CH₂—O-bromobenzaldehyde, wherein p is 1-5;
or when R¹² is

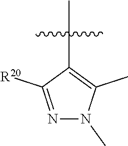

and R¹⁰ is —O-naphthyl substituted with —O— or —S—, then R²⁰ is

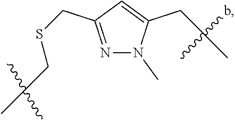

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

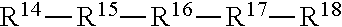

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent
   wherein at least one of R¹⁴-R¹⁸ is present

In some embodiments of the ninth aspect, L′ is hydrogen or methyl. In some embodiments, L′ is hydrogen.

In some embodiments of the ninth aspect, M is O or NH, or is absent.

In some embodiments of the ninth aspect, [ligase ligand moiety] is

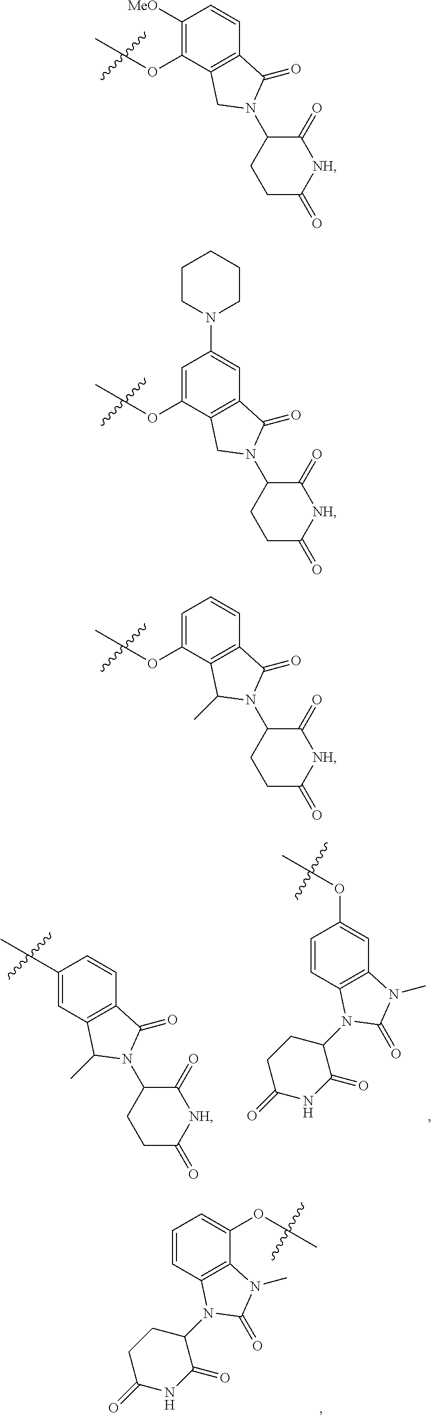

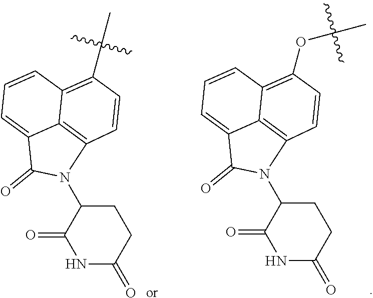 [SMILES: *c1ccc2c3c(cccc13)C(=O)N2C1CCC(=O)NC1=O ; *Oc1ccc2c3c(cccc13)C(=O)N2C1CCC(=O)NC1=O ; "." ; "or"]

In some embodiments of the ninth aspect:
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent;
R¹⁷ is —CH₂(C₂H₄—O)_{y}, (C₂H₄—O)ₓ, (C₃H₆—O)ₓ, or is absent; and
R¹⁸ is C₁₋₆ alkyl, cycloalkyl, CH₂—NH—C(O)—, heterocycloalkyl, or is absent.

In some embodiments of the ninth aspect, R¹⁴ is —C₁₋₆ alkyl, —C₂₋₆ alkenyl, —C₂₋₆ alkynyl, —C(O)—, —SO₂— or is absent. In some embodiments, R¹⁴ is —C₁₋₆ alkyl.

In some embodiments of the ninth aspect, R¹⁵ is cycloalkyl, heterocycloalkyl, aryl, heteroaryl, —C₁₋₆ alkyl-NH—, -cycloalkyl-NH— or is absent. In some embodiments, R¹⁵ is heterocycloalkyl or is absent. In some such embodiments, R¹⁵ is piperazine, bridged piperazine, piperazine N-oxide, piperazine cation,

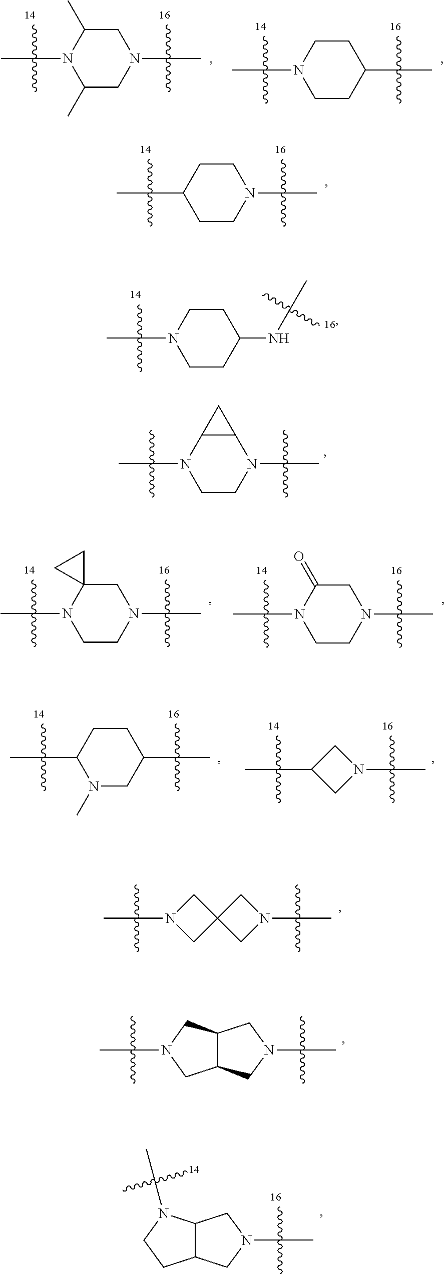

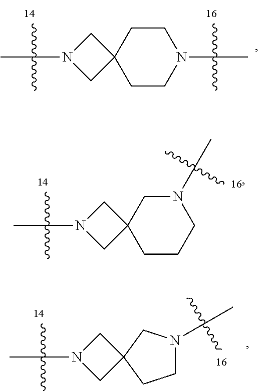 [SMILES: *N1CCC2(CC1)CN(*)C2 ; *N1CCCC2(C1)CN(*)C2 ; *N1CCC2(C1)CN(*)C2]

or is absent;
wherein [symbol] indicates attachment to R¹⁴ and [symbol] indicates attachment to R¹⁶. In some embodiments,
R¹⁵ is piperazine,

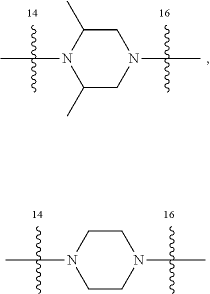

or is absent.

In some embodiments of the ninth aspect, R¹⁶ is —C₁₋₆ alkyl, —CH₂—C(O)—NH—, —CH₂—C(O)—, —C(O)— or is absent.

In some embodiments of the ninth aspect, R¹⁷ is absent.

In some embodiments of the ninth aspect, R¹⁸ is —C₁₋₆ alkyl, cyclobutyl, CH₂—NH—C(O)—, piperazine or is absent.

In some embodiments of the ninth aspect, [linker] is selected from

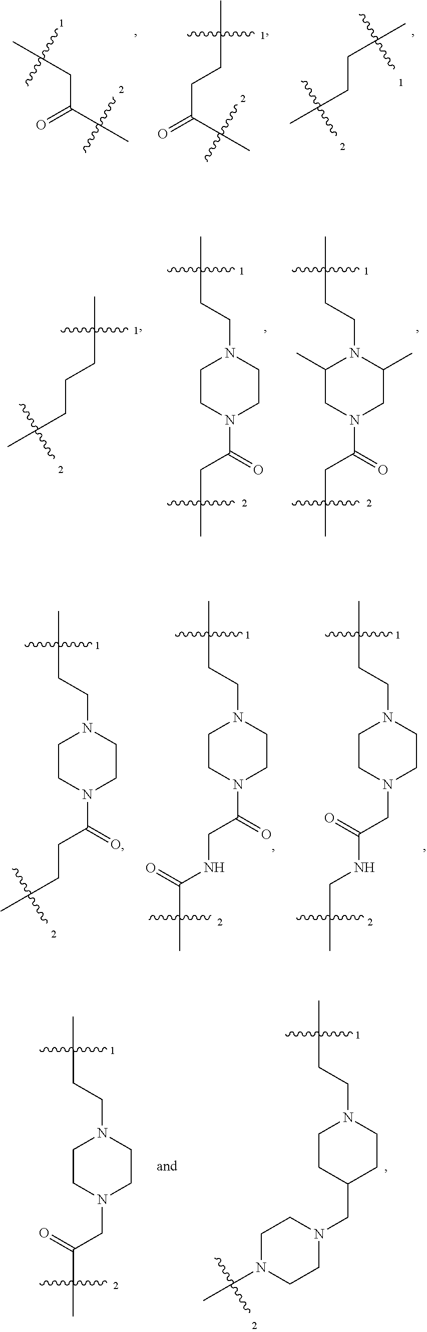

wherein
   [symbol] indicates attachment to [MCL-1 ligand moiety] and
   [symbol] indicates attachment to [ligase ligand moiety].

In some embodiments of the ninth aspect, [linker] is selected from

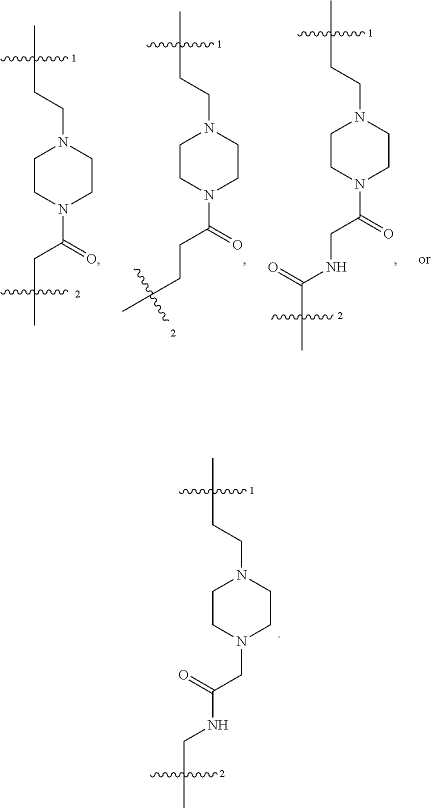

In some embodiments of the ninth aspect, [MCL-1 ligand moiety] is selected from:

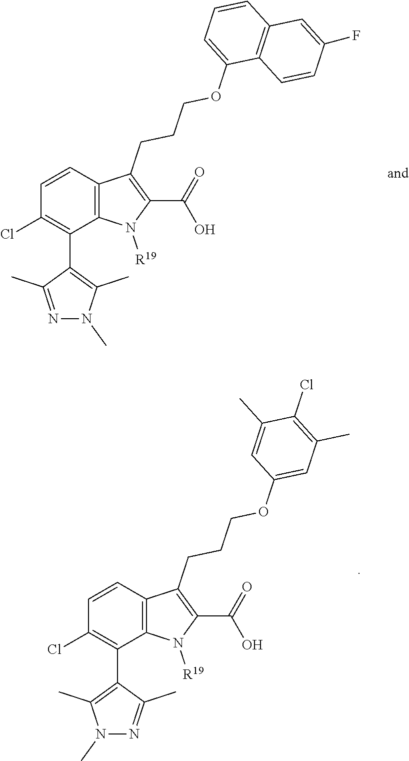

In some embodiments of the ninth aspect, the compound is selected from:

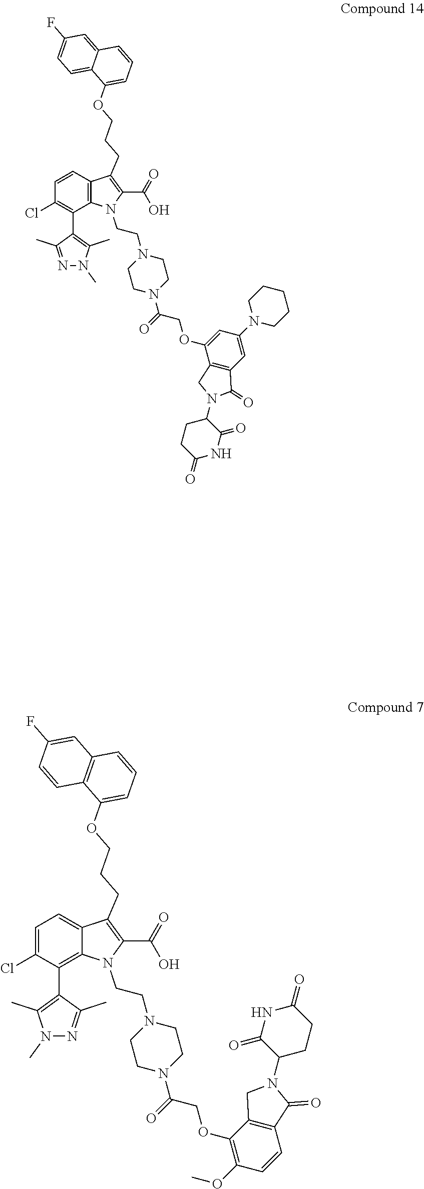 [SMILES: Cc1nn(C)c(C)c1-c1c(Cl)ccc2c(CCCOc3cccc4cc(F)ccc34)c(C(=O)O)n(CCN3CCN(C(=O)COc4cc(N5CCCCC5)cc5c4CN(C4CCC(=O)NC4=O)C5=O)CC3)c12 ; COc1ccc2c(c1OCC(=O)N1CCN(CCn3c(C(=O)O)c(CCCOc4cccc5cc(F)ccc45)c4ccc(Cl)c(-c5c(C)nn(C)c5C)c43)CC1)CN(C1CCC(=O)NC1=O)C2=O]

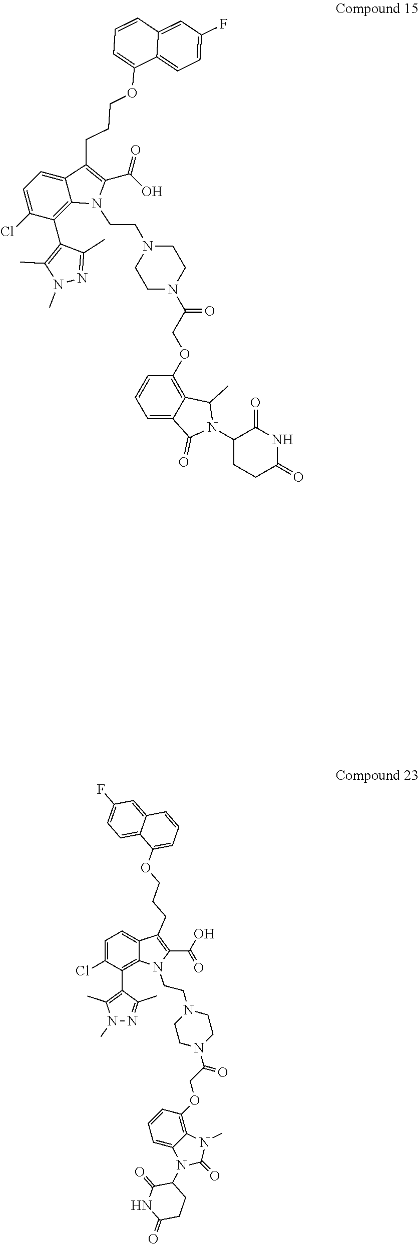 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4C(C)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4n(C)c(=O)n5C4CCC(=O)NC4=O)CC3)c12]

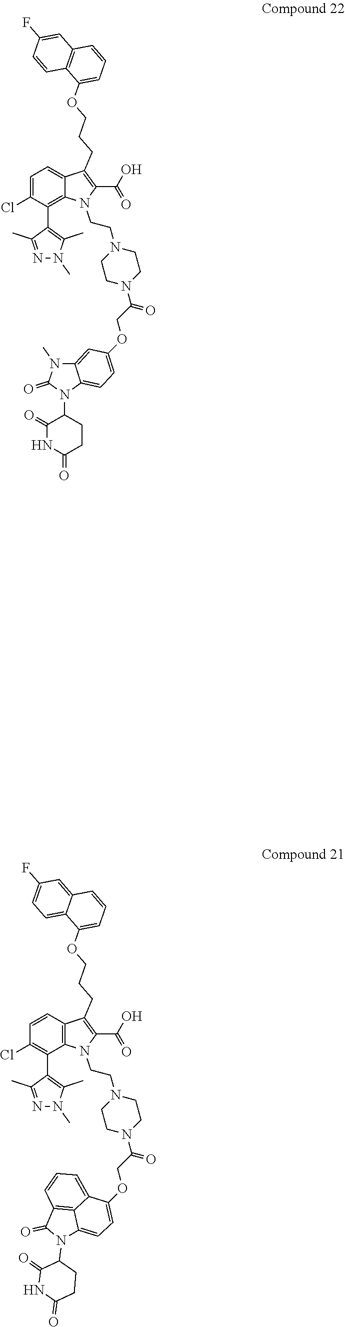 [SMILES: Cc1nn(C)c(C)c1-c1c(Cl)ccc2c(CCCOc3cccc4cc(F)ccc34)c(C(=O)O)n(CCN3CCN(C(=O)COc4ccc5c(c4)n(C)c(=O)n5C4CCC(=O)NC4=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c6c(cccc46)C(=O)N5C4CCC(=O)NC4=O)CC3)c12]

In some embodiments of the ninth aspect, the compound is selected from Compounds 7, 15, 23, 22 and 21. In some embodiments, the compound is Compound 22.

In accordance with a tenth aspect of the present invention, there is provided a compound of formula (I)

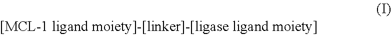

or a salt, solvate, hydrate, isomer or prodrug thereof,
wherein [ligase ligand moiety] is of Formula (VIa) or (VIb):

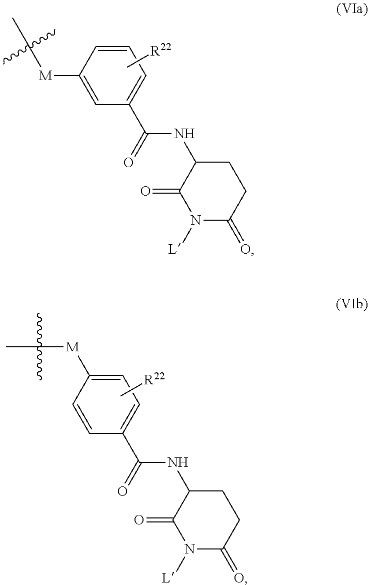

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₅ alkyl; and
   L′ is hydrogen, alkyl, benzyl, acetyl or pivaloyl
wherein [MCL-1 ligand moiety] is a compound of Formula (A):

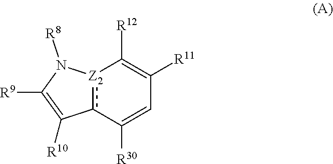

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,
   R¹² is H,

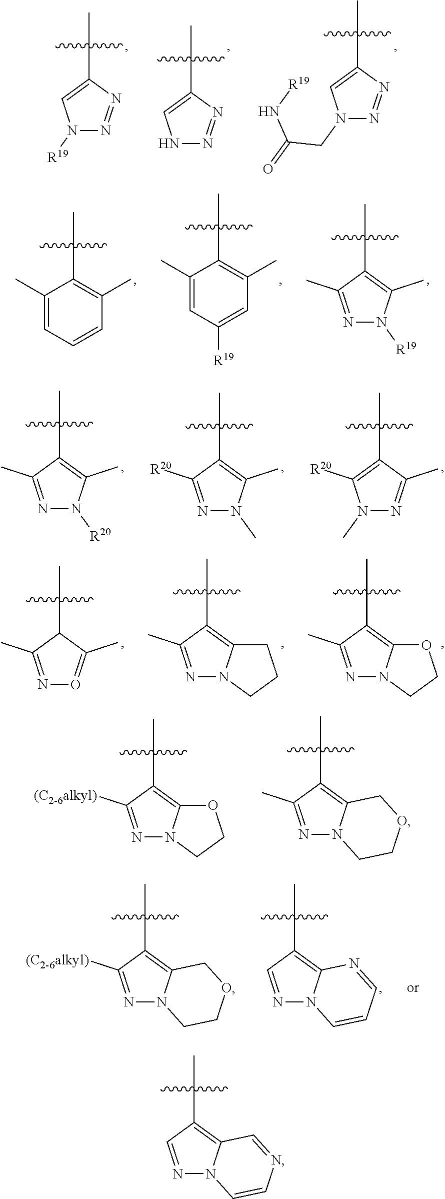

wherein R²⁰ is Me, —CH₂—OMe, —(CH₂CH₂O)ₚ(C₁-C₆ alkyl), —CH₂—O-bromobenzaldehyde, or

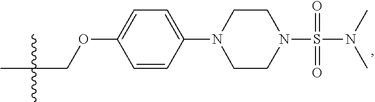

wherein p is 1-5; or
when R¹² is

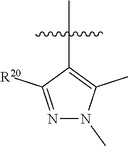

and R¹⁰ is —O-naphthyl substituted with —O— or —S—, then R²⁰ is

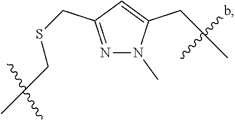

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

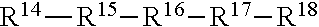

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent wherein at least one of R¹⁴-R¹⁸ is present.

In some embodiments of the tenth aspect, [ligase ligand moiety] is:

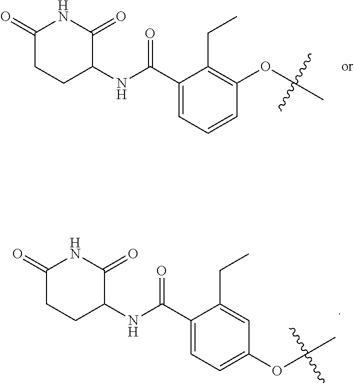 [SMILES: *Oc1cccc(C(=O)NC2CCC(=O)NC2=O)c1CC ; *Oc1ccc(C(=O)NC2CCC(=O)NC2=O)c(CC)c1]

In some embodiments of the tenth aspect,
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent;
R¹⁷ is —CH₂(C₂H₄—O)_{y}, (C₂H₄—O)ₓ, (C₃H₆—O)ₓ, or is absent; and
R¹⁸ is —C₁₋₆ alkyl, cycloalkyl, CH₂—NH—C(O)—, heterocycloalkyl, or is absent.

In some embodiments of the tenth aspect, R¹⁴ is —C₁₋₆ alkyl, —C₂₋₆ alkenyl, —C₂₋₆ alkynyl, —C(O)—, —SO₂— or is absent. In some embodiments, R¹⁴ is —C₁₋₆ alkyl.

In some embodiments of the tenth aspect, R⁵ is cycloalkyl, heterocycloalkyl, aryl, heteroaryl, —C₁₋₆ alkyl-NH—, -cycloalkyl-NH— or is absent. In some embodiments, R⁵ is heterocycloalkyl or is absent. In some embodiments, R⁵ is piperazine.

In some embodiments of the tenth aspect, R¹⁶ is —C(O)—.

In some embodiments of the tenth aspect, R¹⁷ is absent.

In some embodiments of the tenth aspect, R¹⁸ is —C₁₋₆ alkyl.

In some embodiments of the tenth aspect, [linker] is

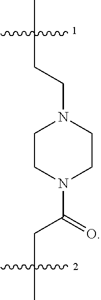 [SMILES: *CCN1CCN(C(=O)C*)CC1]

In some embodiments of the tenth aspect, [MCL-1 ligand moiety] is

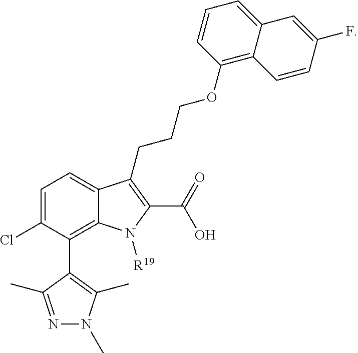

In some embodiments of the tenth aspect, the compound is:

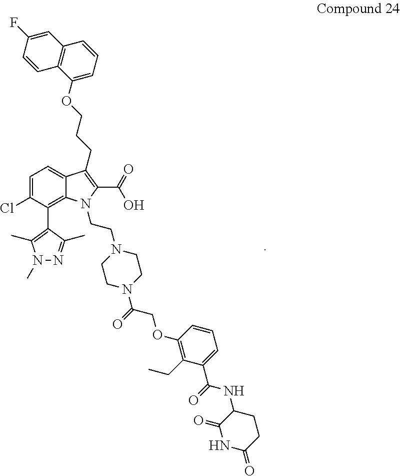 [SMILES: CCc1c(OCC(=O)N2CCN(CCn3c(C(=O)O)c(CCCOc4cccc5cc(F)ccc45)c4ccc(Cl)c(-c5c(C)nn(C)c5C)c43)CC2)cccc1C(=O)NC1CCC(=O)NC1=O]

In some embodiments of any of the first to tenth aspects, T is C═O.

In some embodiments of any of the first to tenth aspects, X₁ and X₂ are O. In other embodiments, X₁ is O and X₂ is S. In other embodiments, X₁ is S and X₂ is O. In other embodiments, X₁ and X₂ are S.

In some embodiments of any of the first to tenth aspects, n is 1.

In some embodiments of any of the first to tenth aspects, unless otherwise specified each alkyl, alkenyl, alkynyl, aryl, heteroaryl and benzyl is unsubstituted.

In an eleventh aspect of the present invention, there is provided a compound selected from:

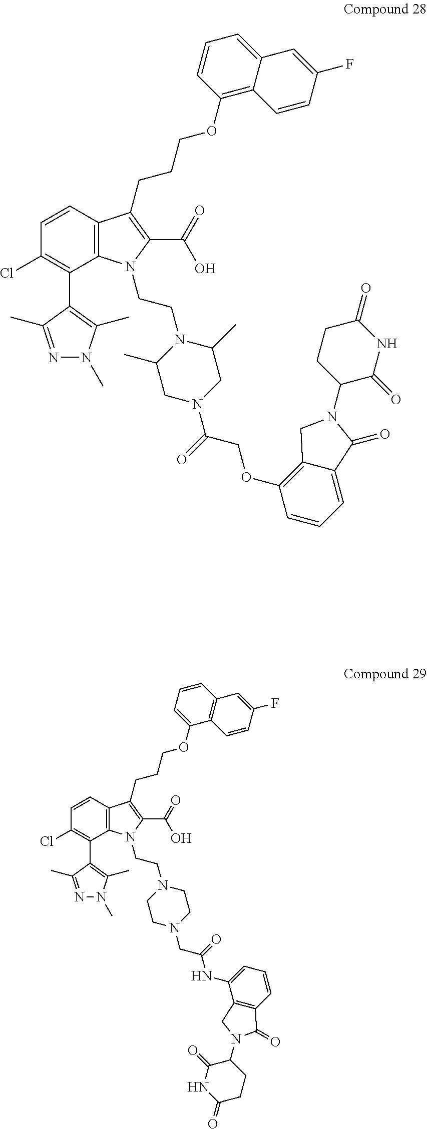 [SMILES: Cc1nn(C)c(C)c1-c1c(Cl)ccc2c(CCCOc3cccc4cc(F)ccc34)c(C(=O)O)n(CCN3C(C)CN(C(=O)COc4cccc5c4CN(C4CCC(=O)NC4=O)C5=O)CC3C)c12 ; Cc1nn(C)c(C)c1-c1c(Cl)ccc2c(CCCOc3cccc4cc(F)ccc34)c(C(=O)O)n(CCN3CCN(CC(=O)Nc4cccc5c4CN(C4CCC(=O)NC4=O)C5=O)CC3)c12]

In a twelfth aspect of the present invention, there is provided a compound selected from:

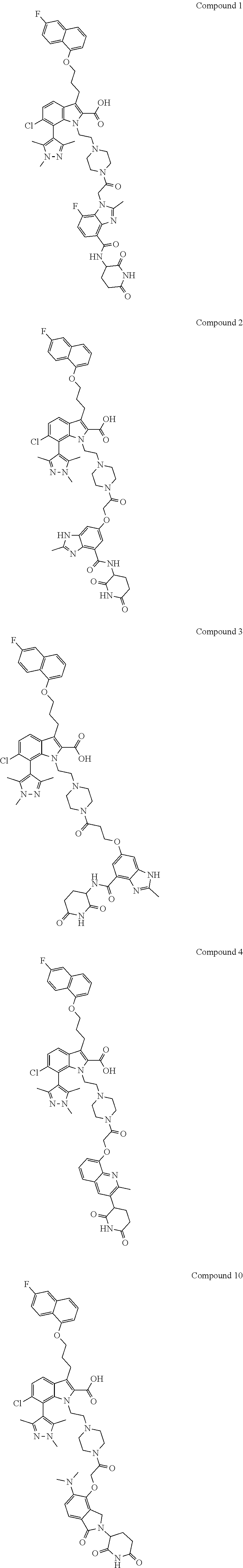

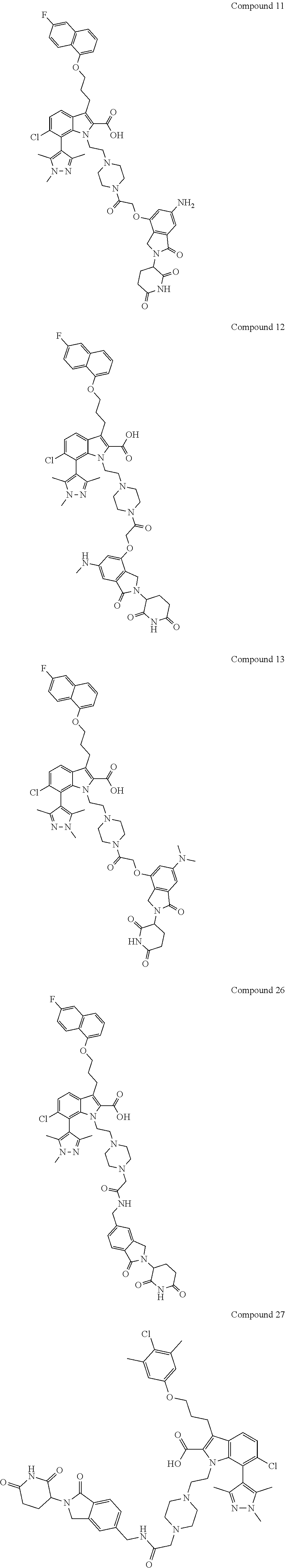

In some embodiments of the twelfth aspect, the compound is selected from Compounds 1, 2, 3, 4, 10, 11, 12, 13 and 26.

In some embodiments of the twelfth aspect, the compound is selected from Compounds 1, 2, 3, 4, 10, 11, 12. In some embodiments, the compound is Compound 12.

In a twelfth aspect of the present invention, there is provided a pharmaceutical composition comprising a compound of the present invention.

In a thirteenth aspect of the present invention, there is provided a compound or pharmaceutical composition of the present invention, for use in medicine.

In a fourteenth aspect of the present invention, there is provided a method of treating cancer in a subject in need thereof, the method comprising administering to the subject an effective amount of a compound or pharmaceutical composition of the present invention.

In some embodiments, the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer. In some embodiments, the cancer is multiple myeloma acute myeloid leukaemia.

In some embodiments, the administration does not result in cytotoxicity in cardiomyocytes in the subject.

In some embodiments, the method further comprises administering at least one additional active agent to the subject. In some embodiments, the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

In a fifteenth aspect of the present invention, there is provided a method of reversing resistance to chemotherapy or targeted cancer therapies in a subject in need thereof, the method comprising administering to the subject an effective amount of a compound or pharmaceutical composition of the present invention.

In a sixteenth aspect of the present invention, there is provided a combined preparation of a compound of the present invention and at least one additional active agent, for simultaneous, separate or sequential use in therapy.

In some embodiments, the at least one additional active agent is an anti-cancer agent selected from eribulin; fulvestrant; midostaurin; an immune checkpoint inhibitor selected from anti-pd-1 antibody, anti-pd-I1 antibody, and anti pd-1/pd-I1 interaction inhibitor; nivolumab; pembrolizumab; atezolizumab; pidilizumab; carfilzomib; venetoclax; cytarabine; anthracyclines; a taxane compound; and hypomethylating agents.

In some embodiments, the therapy is the treatment of cancer.

In a seventeenth aspect of the present invention, there is provided a method of reducing the cardiac cytotoxicity of an MCL-1 inhibitor, comprising coupling a cereblon binding moiety to the MCL-1 inhibitor, wherein the cereblon binding moiety is a [ligase ligand moiety] as defined in any of the embodiments of any one of the first to tenth aspects, and the MCL-1 inhibitor is an [MCL-1 ligand moiety] as defined in any of the embodiments of any one of the first to tenth aspects.

As used herein the term “alkyl” is intended to include both unsubstituted alkyl groups, and alkyl groups which are substituted by one or more additional groups. In some embodiments, the alkyl group is an unsubstituted alkyl group. In some embodiments, the alkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkyl group is a C₁-C₁₂ alkyl, a C₁-C₁₀ alkyl, a C₁-C₅ alkyl, a C₁-C₆ alkyl, or a C₁-C₄ alkyl group. In some embodiments the alkyl group is a linear alkyl group. In some embodiments the alkyl group is an unsubstituted linear alkyl group. In some embodiments the alkyl group is a linear alkyl group which is substituted by one or more groups selected from —OH, —OR^{W}, —NH₂, —NHR^{W}, —NR^{W}₂, —SO₂R^{W}, —C(O)R^{w}, —CN, and —NO₂, wherein each R^{W} is unsubstituted and is independently alkyl, cycloalkyl, alkenyl, cycloalkenyl, aryl, heteroaryl, or benzyl. In some embodiments the alkyl group is a branched alkyl group. In some embodiments the alkyl group is an unsubstituted branched alkyl group. In some embodiments the alkyl group is a branched alkyl group which is substituted by one or more groups selected from —OH, —OR^{w}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “alkenyl” is intended to include both unsubstituted alkenyl groups, and alkenyl groups which are substituted by one or more additional groups. In some embodiments, the alkenyl group is an unsubstituted alkenyl group. In some embodiments, the alkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkenyl group is a C₂-C₁₂ alkenyl, a C₂-C₁₀ alkenyl, a C₂-C₆ alkenyl, a C₂-C₆ alkenyl, or a C₂-C₄ alkenyl group. In some embodiments the alkenyl group is a linear alkenyl group. In some embodiments the alkenyl group is an unsubstituted linear alkenyl group. In some embodiments the alkenyl group is a linear alkenyl group which is substituted by one or more groups selected from —OH, —OR^{w}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkenyl group is a branched alkenyl group. In some embodiments the alkenyl group is an unsubstituted branched alkenyl group. In some embodiments the alkenyl group is a branched alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “alkynyl” is intended to include both unsubstituted alkynyl groups, and alkynyl groups which are substituted by one or more additional groups. In some embodiments, the alkynyl group is an unsubstituted alkynyl group. In some embodiments, the alkynyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkynyl group is a C₂-C₁₂ alkynyl, a C₂-C₁₀ alkynyl, a C₂-C₆ alkynyl, a C₂-C₆ alkynyl, or a C₂-C₄ alkynyl group. In some embodiments the alkynyl group is a linear alkynyl group. In some embodiments the alkynyl group is an unsubstituted linear alkynyl group. In some embodiments the alkynyl group is a linear alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkynyl group is a branched alkynyl group. In some embodiments the alkynyl group is an unsubstituted branched alkynyl group. In some embodiments the alkynyl group is a branched alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “cycloalkyl” is intended to include both unsubstituted cycloalkyl groups, and cycloalkyl groups which are substituted by one or more additional groups. The term “cycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the cycloalkyl group is an unsubstituted cycloalkyl group. In some embodiments, the cycloalkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkyl group is a C₃-C₁₂ cycloalkyl, a C₄-C₁₂ cycloalkyl, a C₅-C₁₂ cycloalkyl, a C₃-C₁₀ cycloalkyl, a C₄-C₁₀ cycloalkyl, a C₅-C₁₀ cycloalkyl, a C₃-C₈ cycloalkyl, a C₄-C₈ cycloalkyl, a C₅-C₈ cycloalkyl, a C₃-C₆ cycloalkyl, a C₄-C₆ cycloalkyl, a C₅-C₆ cycloalkyl, a C₃-C₄ cycloalkyl, or a C₄-C₅ cycloalkyl group.

As used herein the term “cycloalkenyl” is intended to include both unsubstituted cycloalkenyl groups, and cycloalkenyl groups which are substituted by one or more additional groups. In some embodiments, the cycloalkenyl group is an unsubstituted cycloalkenyl group. In some embodiments, the cycloalkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkenyl group is a C₄-C₁₂ cycloalkenyl, a C₅-C₁₂ cycloalkenyl, a C₄-C₁₀ cycloalkenyl, a C₅-C₁₀ cycloalkenyl, a C₄-C₅ cycloalkenyl, a C₅-C₈ cycloalkenyl, a C₄-C₆ cycloalkenyl, a C₅-C₆ cycloalkenyl, or a C₄-C₅ cycloalkenyl group.

As used herein the term “heterocycloalkyl” is intended to include both unsubstituted heterocycloalkyl groups, and heterocycloalkyl groups which are substituted by one or more additional groups. The term “heterocycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the heterocycloalkyl group is a monocyclic ring system, a spirocyclic ring system, or a fused bicyclic ring system. In some embodiments, the heterocycloalkyl group is an unsubstituted heterocycloalkyl group. In some embodiments, the heterocycloalkyl group is substituted by one or more groups selected from —R^{W}, —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, one or more —CH₂— groups of the heterocycloalkyl ring may be replaced with a —C(O)— group, In some embodiments, the heterocycloalkyl group is a C₃-C₁₂ heterocycloalkyl, a C₄-C₁₂ heterocycloalkyl, a C₅-C₁₂ heterocycloalkyl, a C₃-C₁₀ heterocycloalkyl, a C₄-C₁₀ heterocycloalkyl, a C₅-C₁₀ heterocycloalkyl, a C₃-C₅ heterocycloalkyl, a C₄-C₅ heterocycloalkyl, a C₅-C₈ heterocycloalkyl, a C₃-C₆ heterocycloalkyl, a C₄-C₆ heterocycloalkyl, a C₅-C₆ heterocycloalkyl, a C₃-C₄ heterocycloalkyl, or a C₄-C₅ heterocycloalkyl group.

As used herein the term “aryl” is intended to include both unsubstituted aryl groups, and aryl groups which are substituted by one or more additional groups. In some embodiments, the aryl group is an unsubstituted aryl group. In some embodiments, the aryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the aryl group is a C₆-C₁₀ aryl, a C₆-C₈ aryl, or a C₆ aryl.

As used herein the term “heteroaryl” is intended to include both unsubstituted heteroaryl groups, and heteroaryl groups which are substituted by one or more additional groups. In some embodiments, the heteroaryl group is an unsubstituted heteroaryl group. In some embodiments, the heteroaryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the heteroaryl group is a C₆-C₁₀ heteroaryl, a C₆-C₉ heteroaryl, a C₅-C₈ heteroaryl, or a C₆ heteroaryl.

As used herein the term “fused heterocycloalkyl-heteroaryl” is intended to mean a bicyclic ring system in which one ring is a heterocycloalkyl ring and the other is a heteroaryl ring, and in which the two rings share two adjacent atoms. Of the two adjacent atoms shared by the two rings, both may be carbon atoms; both may be heteroatoms (e. g. independently O, N or S); or one may be a carbon atom and the other a heteroatom (e. g. O, N or S). The fused heterocycloalkyl-heteroaryl may be unsubstituted or may be substituted by one or more additional groups. In some embodiments, the fused heterocycloalkyl-heteroaryl group is an unsubstituted cycloalkenyl group. In some embodiments, the fused heterocycloalkyl-heteroaryl group is substituted by one or more groups selected from —OH, —OR^{W}, —NH₂, —NHR^{w}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “benzyl” is intended to include both unsubstituted benzyl groups, and benzyl groups which are substituted by one or more additional groups. In some embodiments, the benzyl group is an unsubstituted benzyl group. In some embodiments, the benzyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects of the invention, all alkyl, alkenyl, alkynyl, cycloalkyl, cycloalkenyl, heterocycloalkyl, aryl, heteroaryl and benzyl groups in the compounds are unsubstituted.

### DETAILED DESCRIPTION OF THE INVENTION

As discussed above, the present invention provides a compound of formula (I)

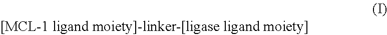

or a salt, solvate, hydrate, isomer or prodrug thereof.

### Ligase Ligand Moieties

Ligase Ligand Moieties with Thalidomide-Type Structure

According to the first and fifth aspects of the present invention, the [ligase ligand moiety] is:

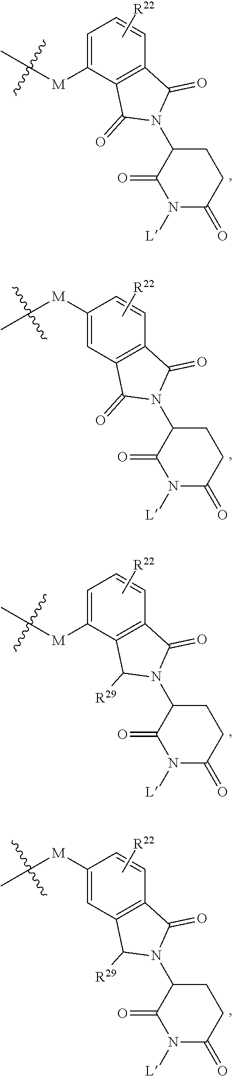

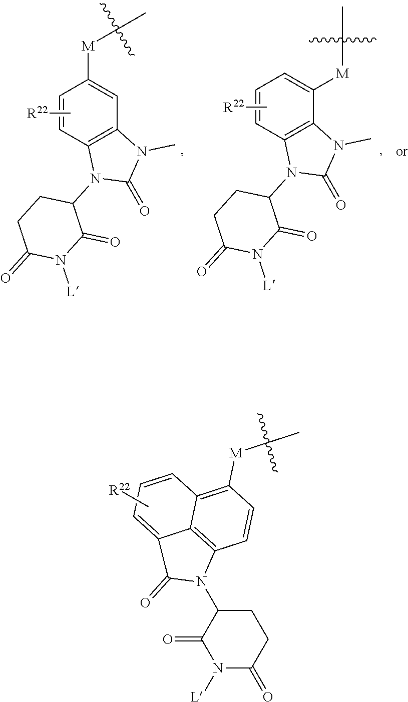

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl;
   R²⁹ s hydrogen or Me; and
   L′ is hydrogen, alkyl, benzyl, acetyl or pivaloyl.

In some embodiments of the first and fifth aspects of the invention, L′ is hydrogen or methyl. In some embodiments, L′ is hydrogen.

In some embodiments of the first and fifth aspects of the invention, M is O or NH, or is absent.

In some embodiments of the first and fifth aspects of the invention, R²⁹ is Me. In other embodiments, R²⁹ is hydrogen.

Examples of the above ligase ligand moieties are shown in Table 1 below:

TABLE 1

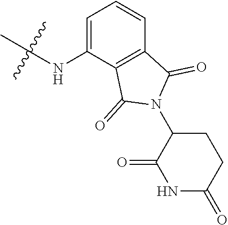 [SMILES: *Nc1cccc2c1C(=O)N(C1CCC(=O)NC1=O)C2=O]

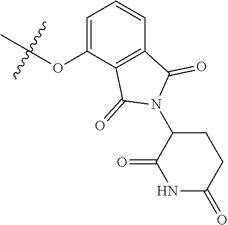 [SMILES: *Oc1cccc2c1C(=O)N(C1CCC(=O)NC1=O)C2=O]

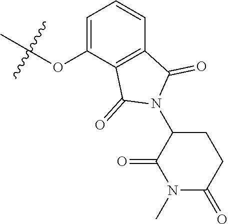 [SMILES: *Oc1cccc2c1C(=O)N(C1CCC(=O)N(C)C1=O)C2=O]

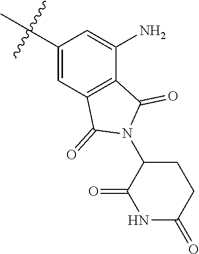 [SMILES: *c1cc(N)c2c(c1)C(=O)N(C1CCC(=O)NC1=O)C2=O]

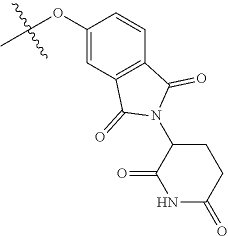 [SMILES: *Oc1ccc2c(c1)C(=O)N(C1CCC(=O)NC1=O)C2=O]

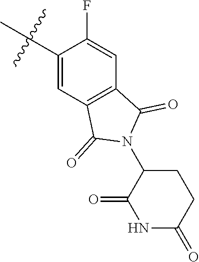 [SMILES: *c1cc2c(cc1F)C(=O)N(C1CCC(=O)NC1=O)C2=O]

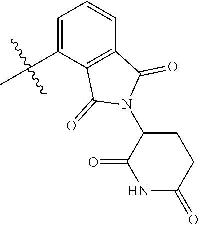 [SMILES: *c1cccc2c1C(=O)N(C1CCC(=O)NC1=O)C2=O]

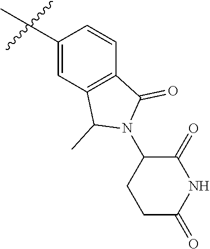 [SMILES: *c1ccc2c(c1)C(C)N(C1CCC(=O)NC1=O)C2=O]

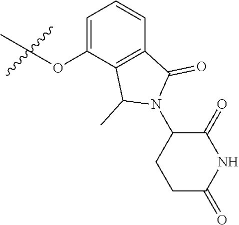 [SMILES: *Oc1cccc2c1C(C)N(C1CCC(=O)NC1=O)C2=O]

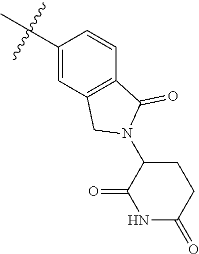 [SMILES: *c1ccc2c(c1)CN(C1CCC(=O)NC1=O)C2=O]

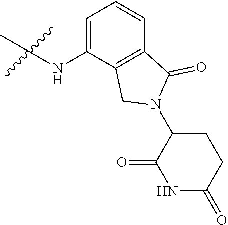 [SMILES: *Nc1cccc2c1CN(C1CCC(=O)NC1=O)C2=O]

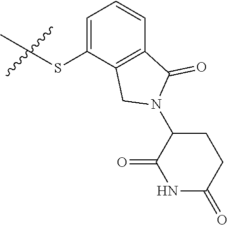 [SMILES: *Sc1cccc2c1CN(C1CCC(=O)NC1=O)C2=O]

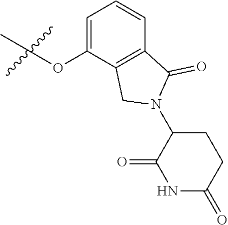 [SMILES: *Oc1cccc2c1CN(C1CCC(=O)NC1=O)C2=O]

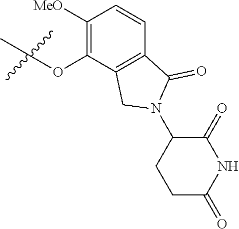 [SMILES: *Oc1c(OC)ccc2c1CN(C1CCC(=O)NC1=O)C2=O]

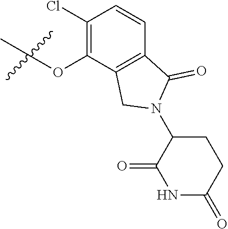 [SMILES: *Oc1c(Cl)ccc2c1CN(C1CCC(=O)NC1=O)C2=O]

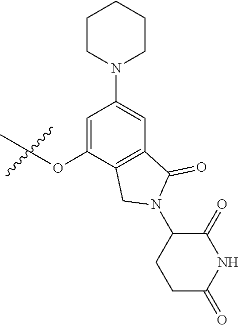 [SMILES: *Oc1cc(N2CCCCC2)cc2c1CN(C1CCC(=O)NC1=O)C2=O]

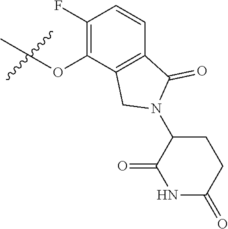 [SMILES: *Oc1c(F)ccc2c1CN(C1CCC(=O)NC1=O)C2=O]

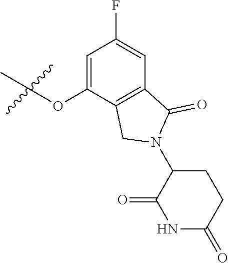 [SMILES: *Oc1cc(F)cc2c1CN(C1CCC(=O)NC1=O)C2=O]

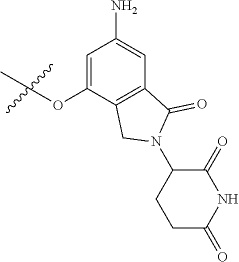 [SMILES: *Oc1cc(N)cc2c1CN(C1CCC(=O)NC1=O)C2=O]

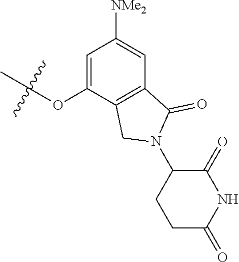 [SMILES: *Oc1cc(N(C)C)cc2c1CN(C1CCC(=O)NC1=O)C2=O]

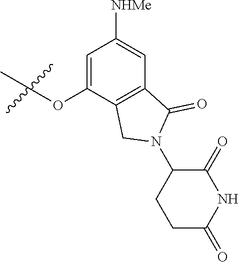 [SMILES: *Oc1cc(NC)cc2c1CN(C1CCC(=O)NC1=O)C2=O]

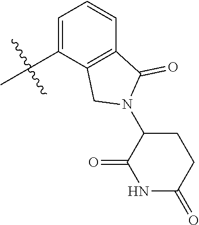 [SMILES: *c1cccc2c1CN(C1CCC(=O)NC1=O)C2=O]

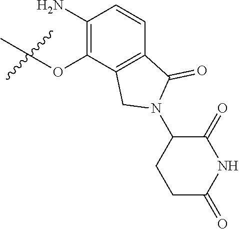 [SMILES: *Oc1c(N)ccc2c1CN(C1CCC(=O)NC1=O)C2=O]

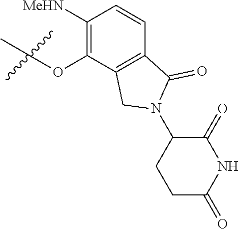 [SMILES: *Oc1c(NC)ccc2c1CN(C1CCC(=O)NC1=O)C2=O]

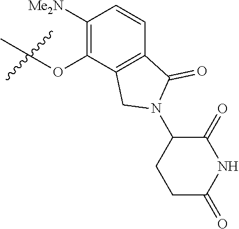 [SMILES: *Oc1c(N(C)C)ccc2c1CN(C1CCC(=O)NC1=O)C2=O]

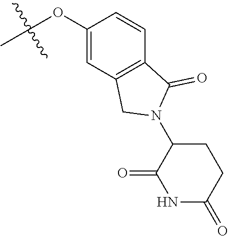 [SMILES: *Oc1ccc2c(c1)CN(C1CCC(=O)NC1=O)C2=O]

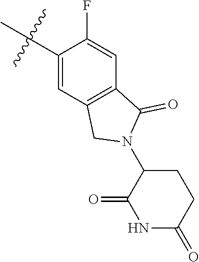 [SMILES: *c1cc2c(cc1F)C(=O)N(C1CCC(=O)NC1=O)C2]

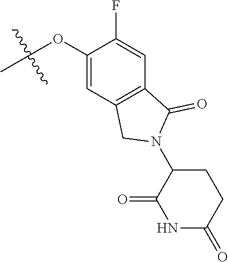 [SMILES: *Oc1cc2c(cc1F)C(=O)N(C1CCC(=O)NC1=O)C2]

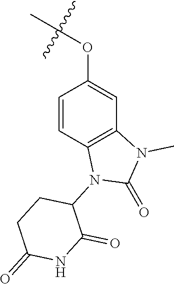 [SMILES: *Oc1ccc2c(c1)n(C)c(=O)n2C1CCC(=O)NC1=O]

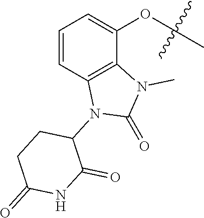 [SMILES: *Oc1cccc2c1n(C)c(=O)n2C1CCC(=O)NC1=O]

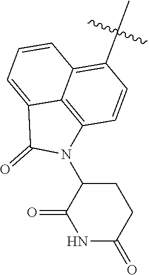 [SMILES: *c1ccc2c3c(cccc13)C(=O)N2C1CCC(=O)NC1=O]

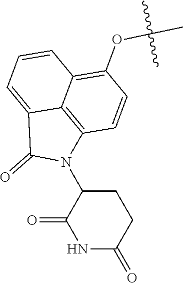 [SMILES: *Oc1ccc2c3c(cccc13)C(=O)N2C1CCC(=O)NC1=O]

In some aspects of the first and fifth aspects of the present invention, [ligase ligand moiety] is

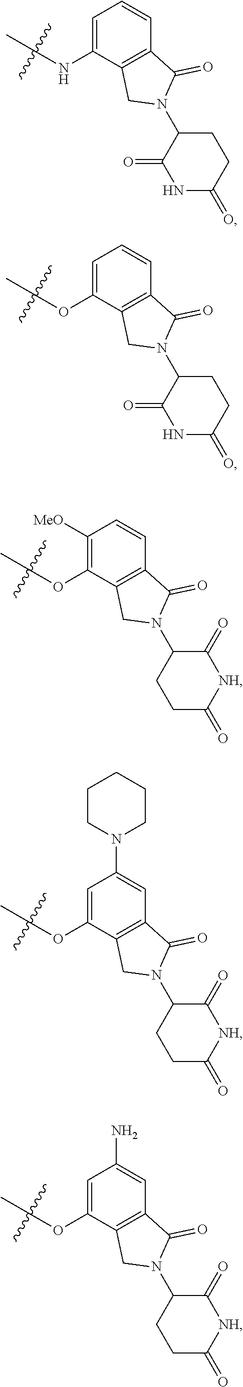 [SMILES: *Nc1cccc2c1CN(C1CCC(=O)NC1=O)C2=O ; *Oc1cccc2c1CN(C1CCC(=O)NC1=O)C2=O ; *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cc(N)cc2c1CN(C1CCC(=O)NC1=O)C2=O]

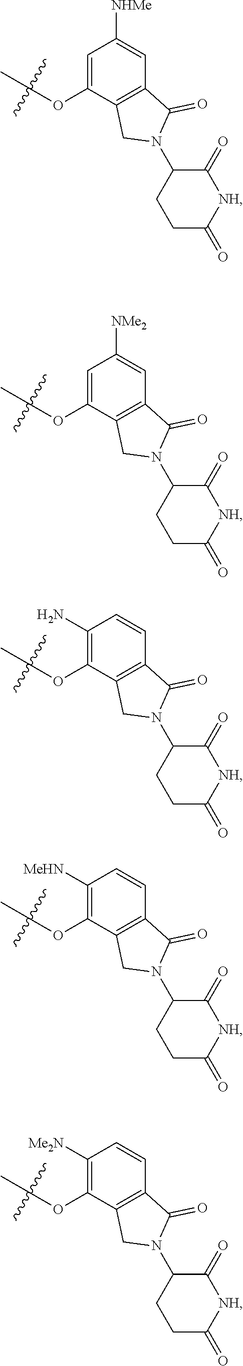 [SMILES: *Oc1cc(NC)cc2c1CN(C1CCC(=O)NC1=O)C2=O ; *Oc1cc(N(C)C)cc2c1CN(C1CCC(=O)NC1=O)C2=O ; *Oc1c(N)ccc2c1CN(C1CCC(=O)NC1=O)C2=O ; *Oc1c(NC)ccc2c1CN(C1CCC(=O)NC1=O)C2=O ; *Oc1c(N(C)C)ccc2c1CN(C1CCC(=O)NC1=O)C2=O]

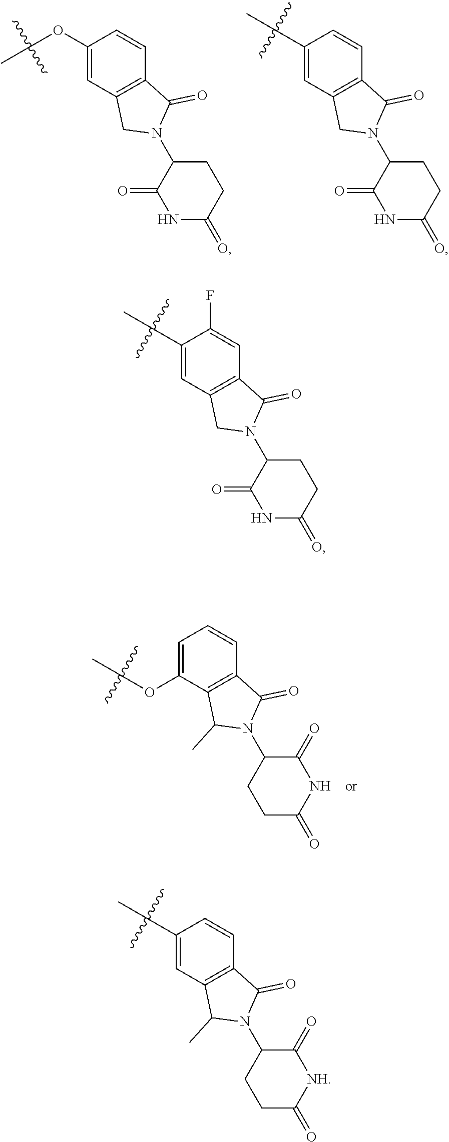 [SMILES: *Oc1ccc2c(c1)CN(C1CCC(=O)NC1=O)C2=O ; *c1ccc2c(c1)CN(C1CCC(=O)NC1=O)C2=O ; *c1cc2c(cc1F)C(=O)N(C1CCC(=O)NC1=O)C2 ; *Oc1cccc2c1C(C)N(C1CCC(=O)NC1=O)C2=O ; *c1ccc2c(c1)C(C)N(C1CCC(=O)NC1=O)C2=O]

In other aspects of the first and fifth aspects of the present invention [ligase ligand moiety] is

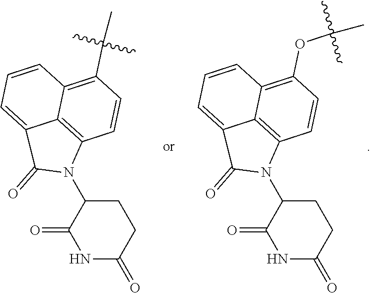 [SMILES: *c1ccc2c3c(cccc13)C(=O)N2C1CCC(=O)NC1=O ; *Oc1ccc2c3c(cccc13)C(=O)N2C1CCC(=O)NC1=O]

In other aspects of the first and fifth aspects of the present invention [ligase ligand moiety] is:

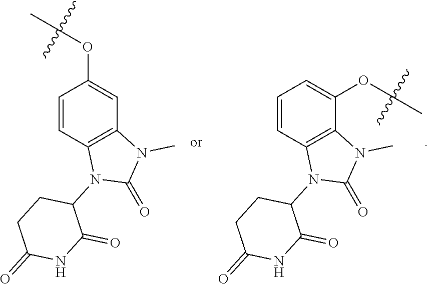 [SMILES: *Oc1ccc2c(c1)n(C)c(=O)n2C1CCC(=O)NC1=O ; *Oc1cccc2c1n(C)c(=O)n2C1CCC(=O)NC1=O]

In the ninth aspect of the present invention, [ligase ligand moiety] is:

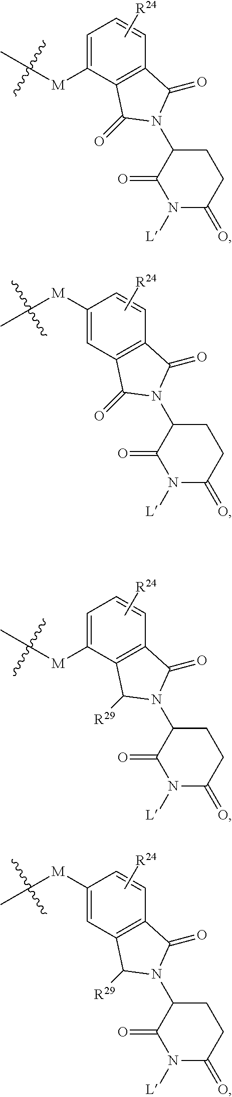

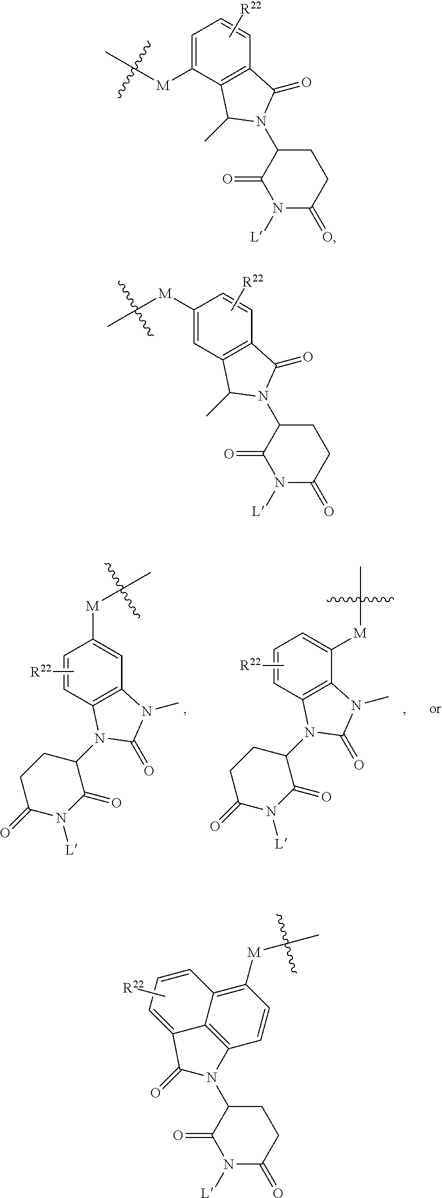

wherein
   M is O, S or NH, or is absent;
   [symbol] indicates attachment to R¹⁸ of the linker;
   R²⁴ is —OMe or heterocycloalkyl;
   R²² is hydrogen, halogen, —OMe, an amino group, heterocycloalkyl, or unsubstituted C₁-C₆ alkyl;
   R²⁹ is hydrogen or Me; and
   L′ is hydrogen, alkyl, benzyl, acetyl or pivaloyl.

In some embodiments of the ninth aspect of the invention, L′ is hydrogen or methyl. In some embodiments, L′ is hydrogen.

In some embodiments of the ninth aspect of the invention, M is O or NH, or is absent.

In some embodiments of the ninth aspect of the invention, R²⁹ is Me. In other embodiments, R²⁹ is hydrogen.

Examples of the above ligase ligand moieties are shown in Table 2 below:

TABLE 2

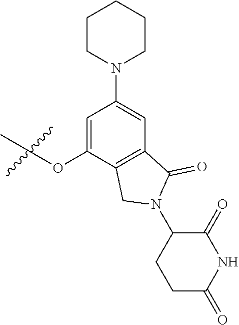 [SMILES: *Oc1cc(N2CCCCC2)cc2c1CN(C1CCC(=O)NC1=O)C2=O]

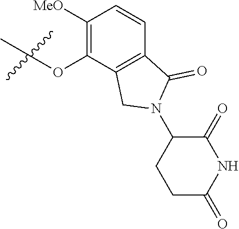 [SMILES: *Oc1c(OC)ccc2c1CN(C1CCC(=O)NC1=O)C2=O]

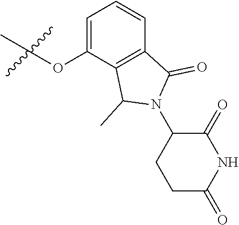 [SMILES: *Oc1cccc2c1C(C)N(C1CCC(=O)NC1=O)C2=O]

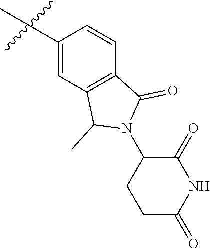 [SMILES: *c1ccc2c(c1)C(C)N(C1CCC(=O)NC1=O)C2=O]

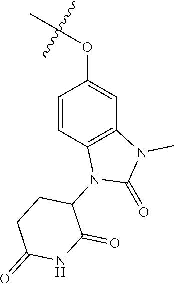 [SMILES: *Oc1ccc2c(c1)n(C)c(=O)n2C1CCC(=O)NC1=O]

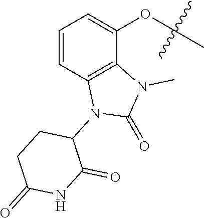 [SMILES: *Oc1cccc2c1n(C)c(=O)n2C1CCC(=O)NC1=O]

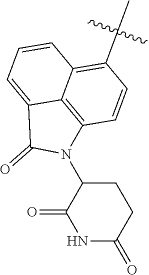 [SMILES: *c1ccc2c3c(cccc13)C(=O)N2C1CCC(=O)NC1=O]

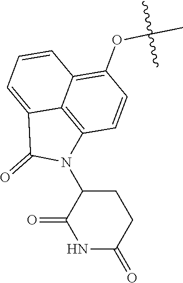 [SMILES: *Oc1ccc2c3c(cccc13)C(=O)N2C1CCC(=O)NC1=O]

### Ligase Ligand Moieties of Formula (II) and Formula (III)

In the fourth and eighth aspects of the invention, the ligase ligand moiety may be of Formula (II) or Formula (III). The synthesis of the ligase ligand moieties of Formula (II) and Formula (III) (as defined above) can be summarized as follows:

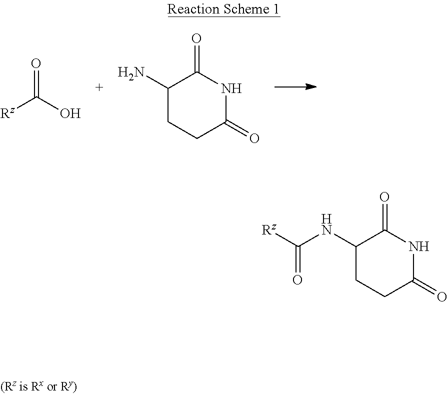

Example ligase ligand moieties of Formula (II) and Formula (III) are shown in Table 3 below. Many of these compounds could be modified to allow attachment to the [linker](for example, by bromination of the aromatic ring followed by attachment—by palladium coupling—of either the [linker] itself, or of a functional group to which the [linker] could be attached).

TABLE 3

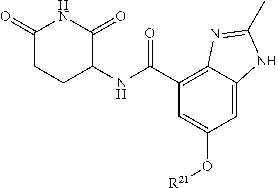

where R²¹ is a bond connected
to R¹⁸ of the linker

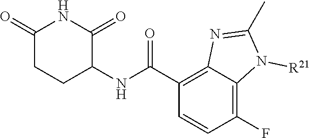

where R²¹ is a bond connected to
R¹⁸ of the linker

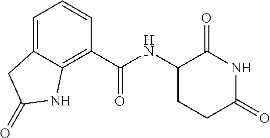 [SMILES: O=C1CCC(NC(=O)c2cccc3c2NC(=O)C3)C(=O)N1]

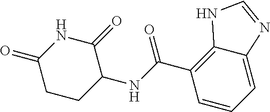 [SMILES: O=C1CCC(NC(=O)c2cccc3nc[nH]c23)C(=O)N1]

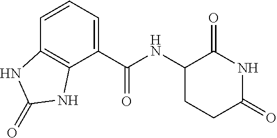 [SMILES: O=C1CCC(NC(=O)c2cccc3[nH]c(=O)[nH]c23)C(=O)N1]

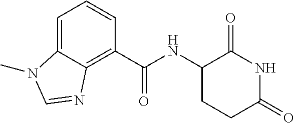 [SMILES: Cn1cnc2c(C(=O)NC3CCC(=O)NC3=O)cccc21]

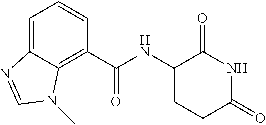 [SMILES: Cn1cnc2cccc(C(=O)NC3CCC(=O)NC3=O)c21]

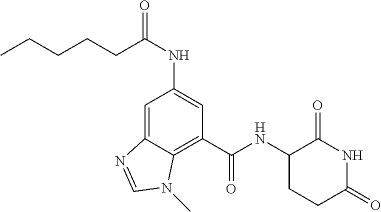 [SMILES: CCCCCC(=O)Nc1cc(C(=O)NC2CCC(=O)NC2=O)c2c(c1)ncn2C]

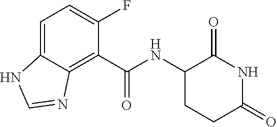 [SMILES: O=C1CCC(NC(=O)c2c(F)ccc3[nH]cnc23)C(=O)N1]

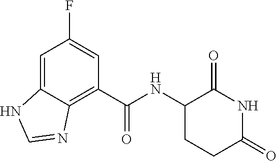 [SMILES: O=C1CCC(NC(=O)c2cc(F)cc3[nH]cnc23)C(=O)N1]

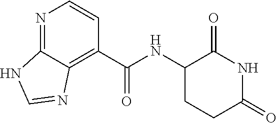 [SMILES: O=C1CCC(NC(=O)c2ccnc3[nH]cnc23)C(=O)N1]

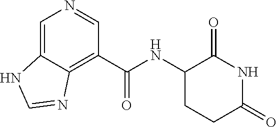 [SMILES: O=C1CCC(NC(=O)c2cncc3[nH]cnc23)C(=O)N1]

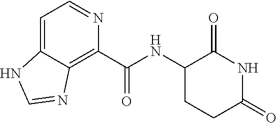 [SMILES: O=C1CCC(NC(=O)c2nccc3[nH]cnc23)C(=O)N1]

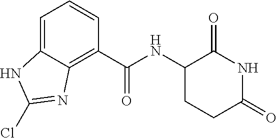 [SMILES: O=C1CCC(NC(=O)c2cccc3[nH]c(Cl)nc23)C(=O)N1]

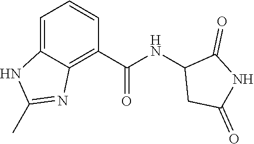 [SMILES: Cc1nc2c(C(=O)NC3CC(=O)NC3=O)cccc2[nH]1]

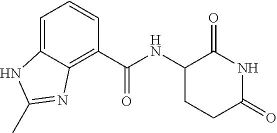 [SMILES: Cc1nc2c(C(=O)NC3CCC(=O)NC3=O)cccc2[nH]1]

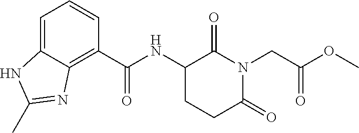

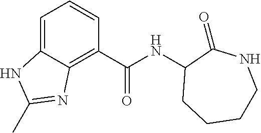 [SMILES: Cc1nc2c(C(=O)NC3CCCCNC3=O)cccc2[nH]1]

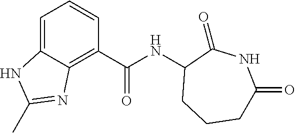 [SMILES: Cc1nc2c(C(=O)NC3CCCC(=O)NC3=O)cccc2[nH]1]

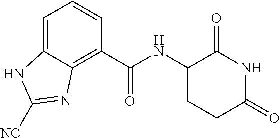 [SMILES: N#Cc1nc2c(C(=O)NC3CCC(=O)NC3=O)cccc2[nH]1]

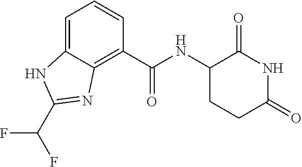 [SMILES: O=C1CCC(NC(=O)c2cccc3[nH]c(C(F)F)nc23)C(=O)N1]

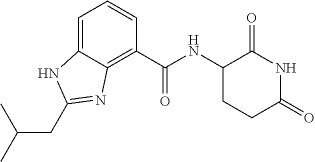 [SMILES: CC(C)Cc1nc2c(C(=O)NC3CCC(=O)NC3=O)cccc2[nH]1]

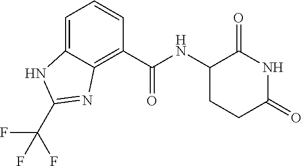 [SMILES: O=C1CCC(NC(=O)c2cccc3[nH]c(C(F)(F)F)nc23)C(=O)N1]

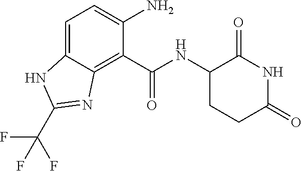 [SMILES: Nc1ccc2[nH]c(C(F)(F)F)nc2c1C(=O)NC1CCC(=O)NC1=O]

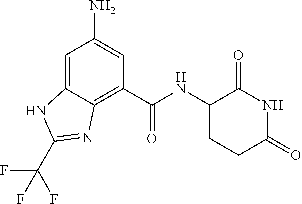 [SMILES: Nc1cc(C(=O)NC2CCC(=O)NC2=O)c2nc(C(F)(F)F)[nH]c2c1]

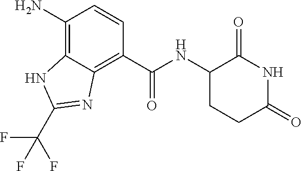 [SMILES: Nc1ccc(C(=O)NC2CCC(=O)NC2=O)c2nc(C(F)(F)F)[nH]c12]

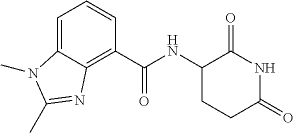 [SMILES: Cc1nc2c(C(=O)NC3CCC(=O)NC3=O)cccc2n1C]

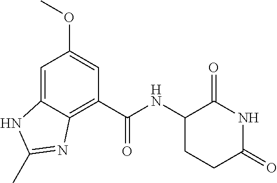 [SMILES: COc1cc(C(=O)NC2CCC(=O)NC2=O)c2nc(C)[nH]c2c1]

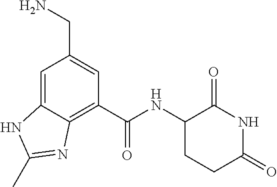 [SMILES: Cc1nc2c(C(=O)NC3CCC(=O)NC3=O)cc(CN)cc2[nH]1]

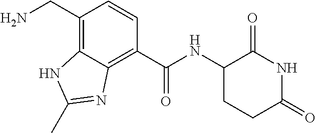 [SMILES: Cc1nc2c(C(=O)NC3CCC(=O)NC3=O)ccc(CN)c2[nH]1]

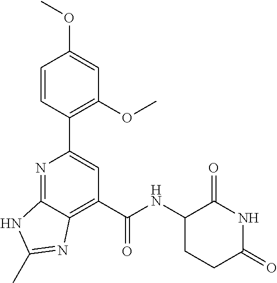 [SMILES: COc1ccc(-c2cc(C(=O)NC3CCC(=O)NC3=O)c3nc(C)[nH]c3n2)c(OC)c1]

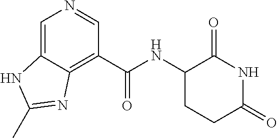 [SMILES: Cc1nc2c(C(=O)NC3CCC(=O)NC3=O)cncc2[nH]1]

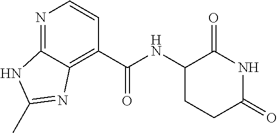 [SMILES: Cc1nc2c(C(=O)NC3CCC(=O)NC3=O)ccnc2[nH]1]

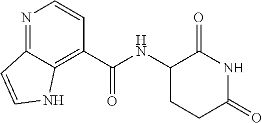 [SMILES: O=C1CCC(NC(=O)c2ccnc3cc[nH]c23)C(=O)N1]

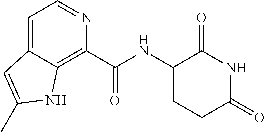 [SMILES: Cc1cc2ccnc(C(=O)NC3CCC(=O)NC3=O)c2[nH]1]

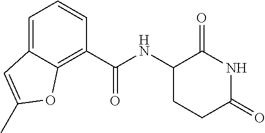 [SMILES: Cc1cc2cccc(C(=O)NC3CCC(=O)NC3=O)c2o1]

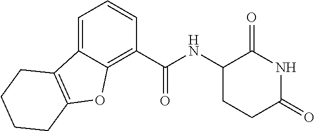 [SMILES: O=C1CCC(NC(=O)c2cccc3c4c(oc23)CCCC4)C(=O)N1]

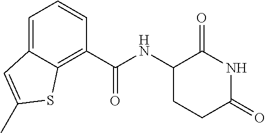 [SMILES: Cc1cc2cccc(C(=O)NC3CCC(=O)NC3=O)c2s1]

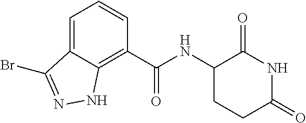 [SMILES: O=C1CCC(NC(=O)c2cccc3c(Br)n[nH]c23)C(=O)N1]

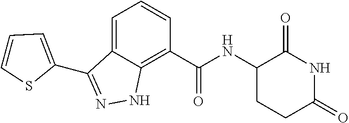 [SMILES: O=C1CCC(NC(=O)c2cccc3c(-c4cccs4)n[nH]c23)C(=O)N1]

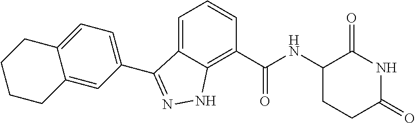 [SMILES: O=C1CCC(NC(=O)c2cccc3c(-c4ccc5c(c4)CCCC5)n[nH]c23)C(=O)N1]

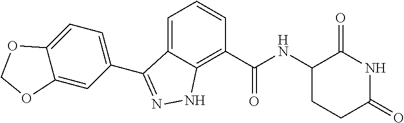 [SMILES: O=C1CCC(NC(=O)c2cccc3c(-c4ccc5c(c4)OCO5)n[nH]c23)C(=O)N1]

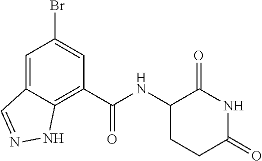 [SMILES: O=C1CCC(NC(=O)c2cc(Br)cc3cn[nH]c23)C(=O)N1]

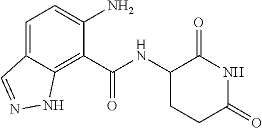 [SMILES: Nc1ccc2cn[nH]c2c1C(=O)NC1CCC(=O)NC1=O]

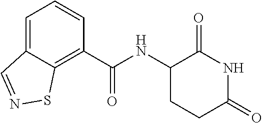 [SMILES: O=C1CCC(NC(=O)c2cccc3cnsc23)C(=O)N1]

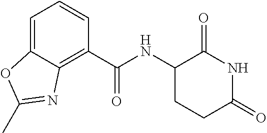 [SMILES: Cc1nc2c(C(=O)NC3CCC(=O)NC3=O)cccc2o1]

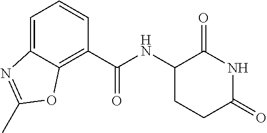 [SMILES: Cc1nc2cccc(C(=O)NC3CCC(=O)NC3=O)c2o1]

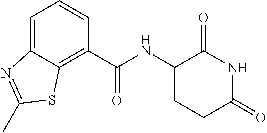 [SMILES: Cc1nc2cccc(C(=O)NC3CCC(=O)NC3=O)c2s1]

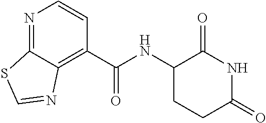 [SMILES: O=C1CCC(NC(=O)c2ccnc3scnc23)C(=O)N1]

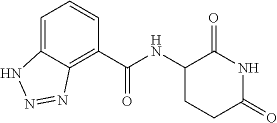 [SMILES: O=C1CCC(NC(=O)c2cccc3[nH]nnc23)C(=O)N1]

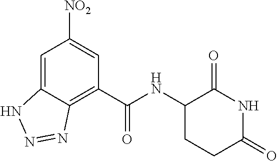 [SMILES: O=C1CCC(NC(=O)c2cc([N+](=O)[O-])cc3[nH]nnc23)C(=O)N1]

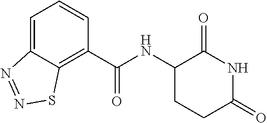 [SMILES: O=C1CCC(NC(=O)c2cccc3nnsc23)C(=O)N1]

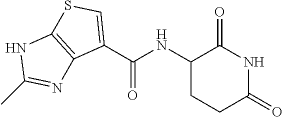 [SMILES: Cc1nc2c(C(=O)NC3CCC(=O)NC3=O)csc2[nH]1]

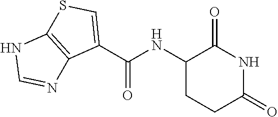 [SMILES: O=C1CCC(NC(=O)c2csc3[nH]cnc23)C(=O)N1]

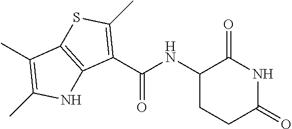 [SMILES: Cc1[nH]c2c(C(=O)NC3CCC(=O)NC3=O)c(C)sc2c1C]

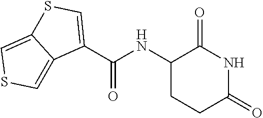 [SMILES: O=C1CCC(NC(=O)c2csc3cscc23)C(=O)N1]

Ligase ligand moieties of Formulae (IV) and (IVa), and of Formulae (VIIa), (VIIb), (VIIc) and (VIId)

In the second and sixth aspects of the present invention, the ligase ligand moiety may be of Formula (IV) or (IVa). The synthesis of the ligase ligand moieties of Formula (IV) (as defined above) can be summarized as follows:

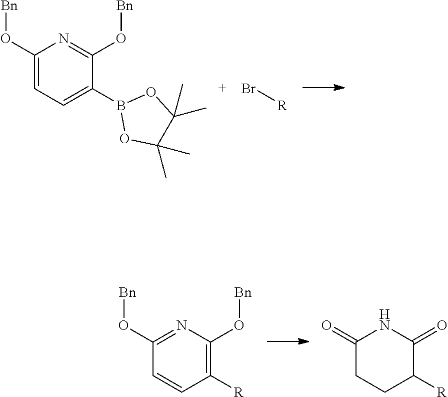

Example ligase ligand moieties of Formula (IV) are shown in Table 4 below. For example,

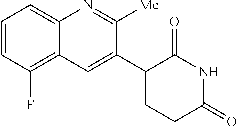 [SMILES: Cc1nc2cccc(F)c2cc1C1CCC(=O)NC1=O]

could be modified to allow attachment to the [linker](e.g. by nucleophilic aromatic substitution; or by exchange of fluorine for bromine followed by attachment—by palladium coupling—of either the [linker] itself, or of a functional group to which the [linker] could be attached).

TABLE 4

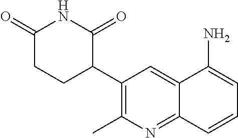 [SMILES: Cc1nc2cccc(N)c2cc1C1CCC(=O)NC1=O]

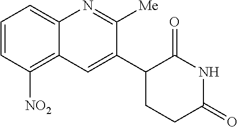 [SMILES: Cc1nc2cccc([N+](=O)[O-])c2cc1C1CCC(=O)NC1=O]

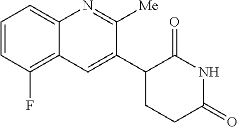 [SMILES: Cc1nc2cccc(F)c2cc1C1CCC(=O)NC1=O]

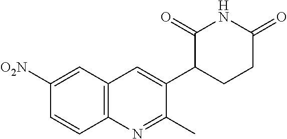 [SMILES: Cc1nc2ccc([N+](=O)[O-])cc2cc1C1CCC(=O)NC1=O]

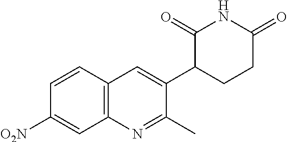 [SMILES: Cc1nc2cc([N+](=O)[O-])ccc2cc1C1CCC(=O)NC1=O]

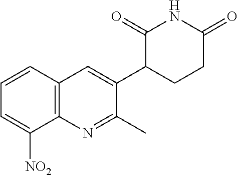 [SMILES: Cc1nc2c([N+](=O)[O-])cccc2cc1C1CCC(=O)NC1=O]

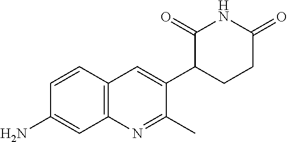 [SMILES: Cc1nc2cc(N)ccc2cc1C1CCC(=O)NC1=O]

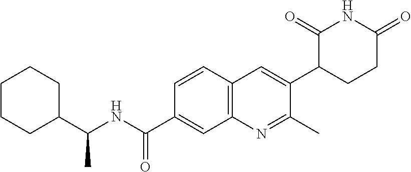 [SMILES: Cc1nc2cc(C(=O)N[C@@H](C)C3CCCCC3)ccc2cc1C1CCC(=O)NC1=O]

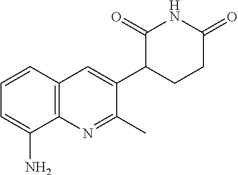 [SMILES: Cc1nc2c(N)cccc2cc1C1CCC(=O)NC1=O]

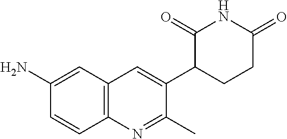 [SMILES: Cc1nc2ccc(N)cc2cc1C1CCC(=O)NC1=O]

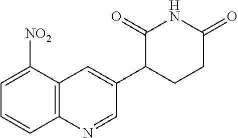 [SMILES: O=C1CCC(c2cnc3cccc([N+](=O)[O-])c3c2)C(=O)N1]

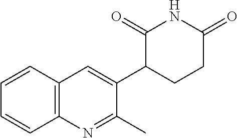 [SMILES: Cc1nc2ccccc2cc1C1CCC(=O)NC1=O]

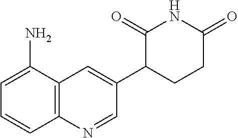 [SMILES: Nc1cccc2ncc(C3CCC(=O)NC3=O)cc12]

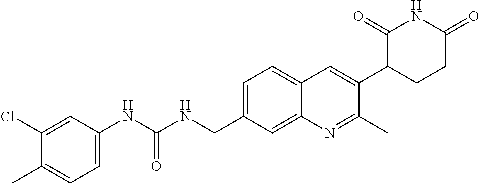 [SMILES: Cc1ccc(NC(=O)NCc2ccc3cc(C4CCC(=O)NC4=O)c(C)nc3c2)cc1Cl]

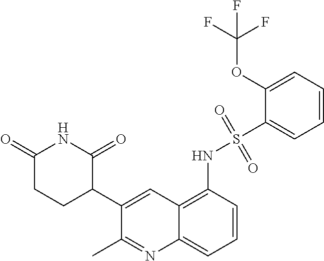 [SMILES: Cc1nc2cccc(NS(=O)(=O)c3ccccc3OC(F)(F)F)c2cc1C1CCC(=O)NC1=O]

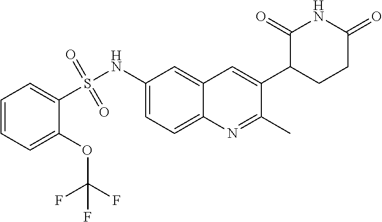 [SMILES: Cc1nc2ccc(NS(=O)(=O)c3ccccc3OC(F)(F)F)cc2cc1C1CCC(=O)NC1=O]

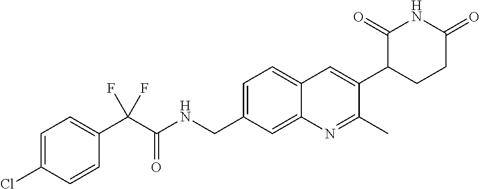 [SMILES: Cc1nc2cc(CNC(=O)C(F)(F)c3ccc(Cl)cc3)ccc2cc1C1CCC(=O)NC1=O]

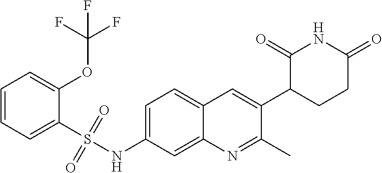 [SMILES: Cc1nc2cc(NS(=O)(=O)c3ccccc3OC(F)(F)F)ccc2cc1C1CCC(=O)NC1=O]

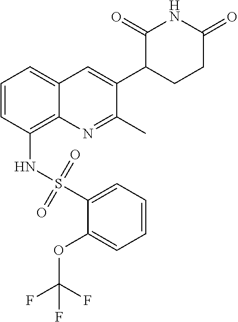 [SMILES: Cc1nc2c(NS(=O)(=O)c3ccccc3OC(F)(F)F)cccc2cc1C1CCC(=O)NC1=O]

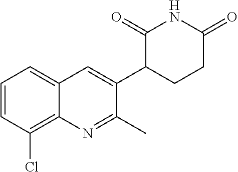 [SMILES: Cc1nc2c(Cl)cccc2cc1C1CCC(=O)NC1=O]

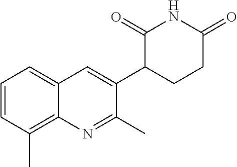 [SMILES: Cc1nc2c(C)cccc2cc1C1CCC(=O)NC1=O]

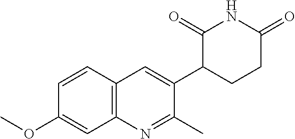 [SMILES: COc1ccc2cc(C3CCC(=O)NC3=O)c(C)nc2c1]

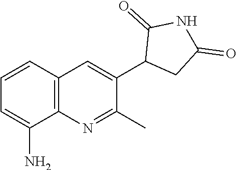 [SMILES: Cc1nc2c(N)cccc2cc1C1CC(=O)NC1=O]

In the second and sixth aspects of the present invention, the ligase ligand moiety may be of Formula (VIIa), (VIIb), (VIIc) or (VIId). Example ligase ligand moieties of Formula (VIIa, (VIIb), (VIIc) and (VIId) are shown in Table 5 below:

TABLE 5

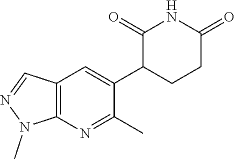 [SMILES: Cc1nc2c(cnn2C)cc1C1CCC(=O)NC1=O]

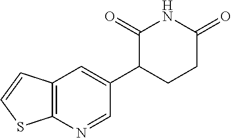 [SMILES: O=C1CCC(c2cnc3sccc3c2)C(=O)N1]

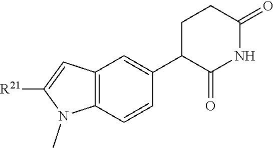

The synthesis of these compounds is summarized in Steps 1-4 below:

### Step 1:

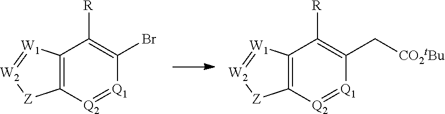

To a solution of bromoarene (1 equiv) in dioxane were added KOAc (2 equiv), ((1-(tert-butoxy)vinyl)oxy)(tert-butyl)dimethylsilane (4 equiv) and Pd[P(o-Tol)₃]₂Cl₂ (0.2 equiv) under inert gas and the reaction mixture was stirred at 130° C. for 48 h. The reaction mixture was filtered through Celite, concentrated under reduced pressure and purified by flash column chromatography to give appropriate tert-butyl arylacetate.

### Step 2:

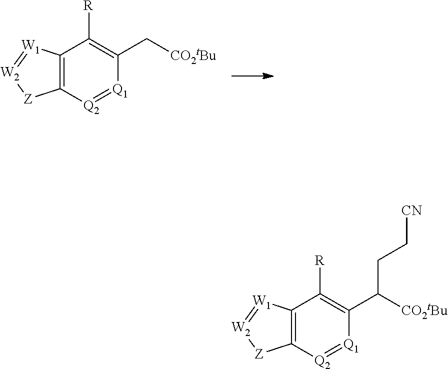

To a solution of appropriate tert-butyl 2-(quinolin-3-yl)acetate (1 equiv) in DMF were added K₂CO₃ (1 equiv), benzyltriethylammonium chloride (1 equiv) and acrylonitrile (1 equiv) and the reaction mixture was stirred at RT for 16 h. The reaction mixture was diluted with water and the product was extracted with ethyl acetate. Combined organic phases were dried over Na₂SO₄, concentrated under reduced pressure and purified by flash column chromatography.

### Step 3:

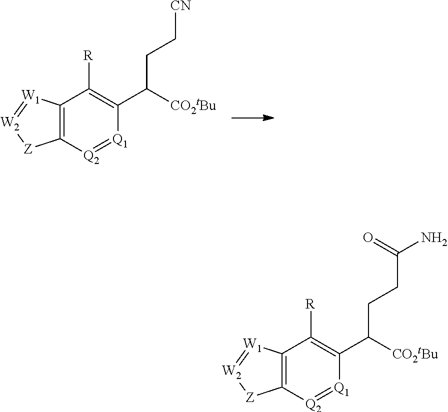

To an ice cold solution of appropriate tert-butyl 4-cyano-2-(quinolin-3-yl)butanoate (1 equiv) in DMSO were added H₂O₂ (5 equiv) and K₂CO₃ (0.1 equiv). The reaction mixture was warmed to RT and stirred for 16 h. The reaction mixture was diluted with water and the product was extracted with ethyl acetate. Combined organic phases were dried over Na₂SO₄, concentrated under reduced pressure and purified by flash column chromatography.

### Step 4:

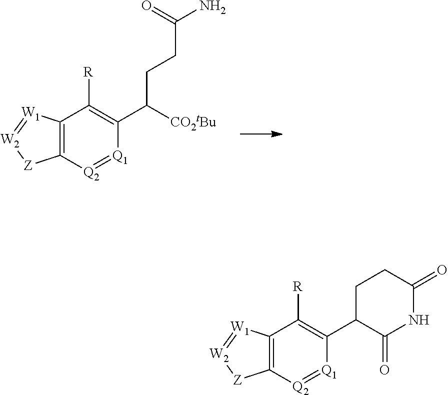

In a vial were placed appropriate tert-butyl 5-amino-2-(2-methylquinolin-3-yl)-5-oxopentanoate (1 equiv), p-toluenesulfonic acid (5-10 equiv) and ACN and the reaction mixture was stirred at 80° C. for 2-48 h. The mixture was concentrated under reduced pressure and purified by flash column chromatography or preparative HPLC.

Ligase ligand moieties of Formula (Va) and Formula (Vb), and Formula (IIa) and Formula (IIb)

In the third and seventh aspects of the invention, the ligase ligand moiety may be of Formulae (Va), (Vb), (IIa) and (IIb) (as defined above). The synthesis of the ligase ligand moieties of Formulae (Va), (Vb), (IIa) and (IIb) can be summarized in the following general procedure (carried out under Synthetic Conditions D, E, F or G, as set out below:

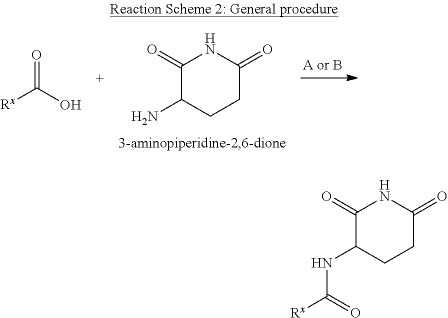

### Synthetic Conditions D

An appropriate acid (R^{x}COOH in the above reaction scheme) (1.1 eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). Water (2×DMF volume) was added and the obtained solution was extracted with dichloromethane (3×DMF volume). The combined organic layers were washed with water, dried over Na₂SO₄, and concentrated under reduced pressure. The crude product was purified by preparative HPLC or by column chromatography.

### Synthetic Conditions E

An appropriate acid R^{x}COOH in the above reaction scheme) (1 eq) and EDC (1.2 eq) were added to a solution of 3-aminopiperidine-2,6-dione (hydrochloride salt, 1.1 eq), triethylamine (1.2 eq) and N-hydroxybenzotriazole (1.2 eq) in DMA (0.5 M). The reaction mixture was stirred overnight at rt. Water (2×DMA volume) was added and obtained mixture was extracted with dichloromethane (3×DMA volume). The combined organic layers were washed with water, dried over Na₂SO₄, and concentrated under reduced pressure. The crude product was isolated by preparative HPLC or by column chromatography.

### Synthetic Conditions F

To a solution of appropriate acid (R^{x}COOH in the above reaction scheme) (1 eq) and HATU (1.5 eq) in dry DMF were added 3-aminopiperidine-2,6-dione (hydrochloride salt, 1.2 eq) and DIPEA (3 eq). The reaction mixture was stirred overnight at rt. The crude product was purified by preparative HPLC or/and by preparative TLC.

### Synthetic Conditions G

To a solution of appropriate acid (R^{x}COOH in the above reaction scheme) (1 eq) 3-aminopiperidine-2,6-dione (hydrochloride salt, 1.2 eq) and DMAP (0.1 eq.) in an inert atmosphere in dry DMF were added DIPEA (2.2 eq.) and HATU (1.5 eq) in dry DMF. The reaction mixture was stirred overnight at rt. The crude product was purified by preparative HPLC or/and by preparative TLC.

### Example Method 1: Formation of Chlorinated R^{x} Group of R^{x}COOH (or its Ester R^{x}COOR^{y})

NCS (1.1 eq) was added to a solution of an appropriate starting material (1 eq) in DMF (0.5 M) and the reaction mixture was stirred for 2 h at room temperature (20-25° C.). The reaction mixture was poured into water (2×DMF volume) and occurred precipitate was filtered. The solids were washed with water and dried in vacuum to give the acid, ROOH.

### Example Method 2: Synthesis of R^{x}COOH from Corresponding Ester R^{x}COOR^{y})

LiOH (1.1 eq) was added to a solution of an appropriate ester (1 eq) in THF:water mixture (3:1 or 5:1, 85 mM) and the resulting mixture was stirred overnight at room temperature (20-25° C.). The mixture was concentrated under reduced pressure, diluted with water, and acidified with concentrated HCl to pH=2-3. The precipitate was filtered, washed with water, and dried in vacuum to give the target carboxylic acid.

### Example Method 3: Formation of Acetylated R^{x} Group of R^{x}COOR^{y}

A mixture of an appropriate amine (1 eq.), Ac₂O (3 eq.), and DMAP (0.2 eq.) in dioxane (0.2 M) was heated to 80° C. for 2 h. Upon completion, the mixture was cooled down to room temperature (20-25° C.) and concentrated under reduced pressure. The residue was diluted with water (1× dioxane volume) and extracted with EtOAc (3× dioxane volume). The organic layers were washed with water, brine, dried over Na₂SO₄, and evaporated to dryness to afford an acylated product typically used without further purification.

Example ligase ligand moieties of Formula (Va) and Formula (Vb) are shown in Table 6 below. Many of these compounds could be modified to allow attachment to the [linker](e.g. by C—H bond activation).

TABLE 6

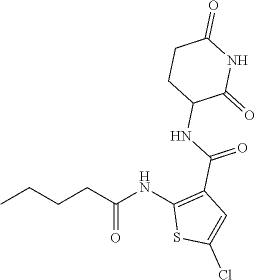 [SMILES: CCCCC(=O)Nc1sc(Cl)cc1C(=O)NC1CCC(=O)NC1=O]

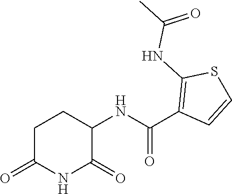 [SMILES: CC(=O)Nc1sccc1C(=O)NC1CCC(=O)NC1=O]

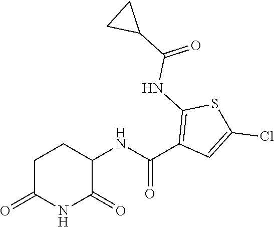 [SMILES: O=C1CCC(NC(=O)c2cc(Cl)sc2NC(=O)C2CC2)C(=O)N1]

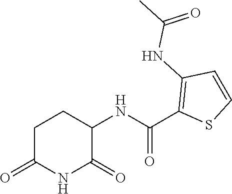 [SMILES: CC(=O)Nc1ccsc1C(=O)NC1CCC(=O)NC1=O]

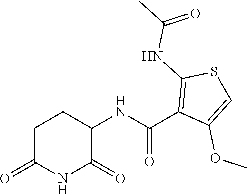 [SMILES: COc1csc(NC(C)=O)c1C(=O)NC1CCC(=O)NC1=O]

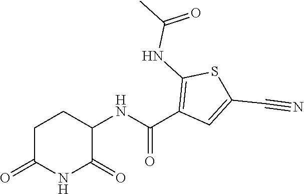 [SMILES: CC(=O)Nc1sc(C#N)cc1C(=O)NC1CCC(=O)NC1=O]

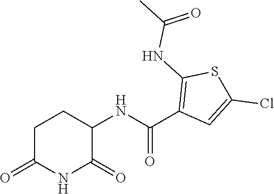 [SMILES: CC(=O)Nc1sc(Cl)cc1C(=O)NC1CCC(=O)NC1=O]

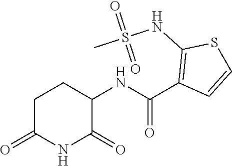 [SMILES: CS(=O)(=O)Nc1sccc1C(=O)NC1CCC(=O)NC1=O]

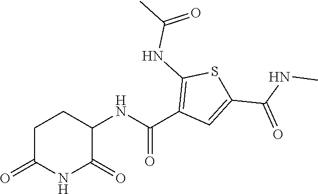 [SMILES: CNC(=O)c1cc(C(=O)NC2CCC(=O)NC2=O)c(NC(C)=O)s1]

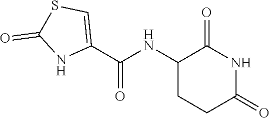 [SMILES: O=C1CCC(NC(=O)c2csc(=O)[nH]2)C(=O)N1]

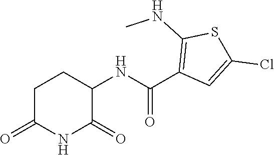 [SMILES: CNc1sc(Cl)cc1C(=O)NC1CCC(=O)NC1=O]

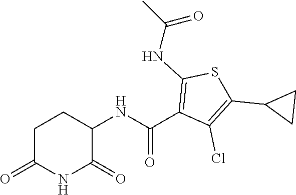 [SMILES: CC(=O)Nc1sc(C2CC2)c(Cl)c1C(=O)NC1CCC(=O)NC1=O]

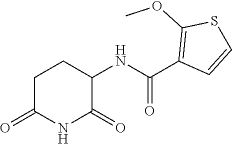 [SMILES: COc1sccc1C(=O)NC1CCC(=O)NC1=O]

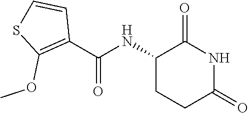 [SMILES: COc1sccc1C(=O)N[C@H]1CCC(=O)NC1=O]

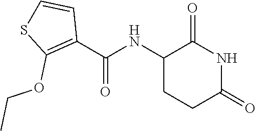 [SMILES: CCOc1sccc1C(=O)NC1CCC(=O)NC1=O]

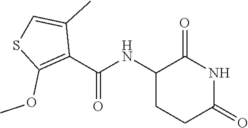 [SMILES: COc1scc(C)c1C(=O)NC1CCC(=O)NC1=O]

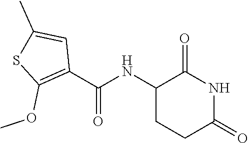 [SMILES: COc1sc(C)cc1C(=O)NC1CCC(=O)NC1=O]

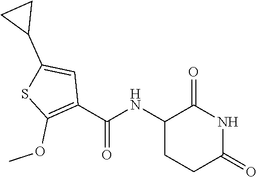 [SMILES: COc1sc(C2CC2)cc1C(=O)NC1CCC(=O)NC1=O]

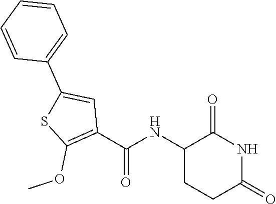 [SMILES: COc1sc(-c2ccccc2)cc1C(=O)NC1CCC(=O)NC1=O]

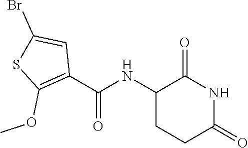 [SMILES: COc1sc(Br)cc1C(=O)NC1CCC(=O)NC1=O]

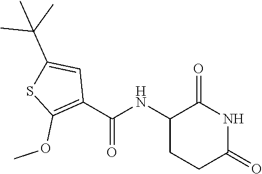 [SMILES: COc1sc(C(C)(C)C)cc1C(=O)NC1CCC(=O)NC1=O]

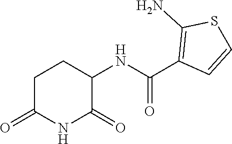 [SMILES: Nc1sccc1C(=O)NC1CCC(=O)NC1=O]

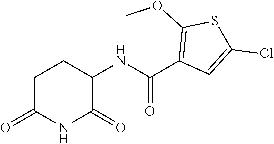 [SMILES: COc1sc(Cl)cc1C(=O)NC1CCC(=O)NC1=O]

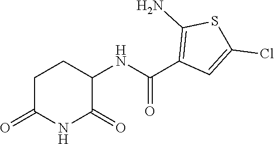 [SMILES: Nc1sc(Cl)cc1C(=O)NC1CCC(=O)NC1=O]

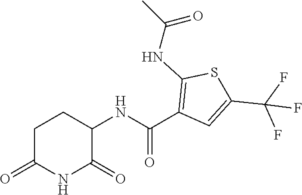 [SMILES: CC(=O)Nc1sc(C(F)(F)F)cc1C(=O)NC1CCC(=O)NC1=O]

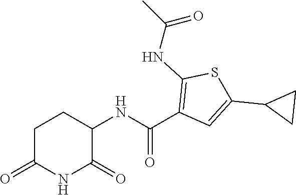 [SMILES: CC(=O)Nc1sc(C2CC2)cc1C(=O)NC1CCC(=O)NC1=O]

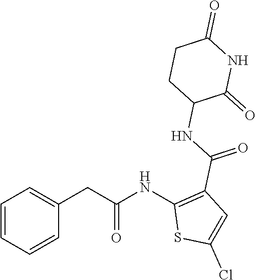 [SMILES: O=C1CCC(NC(=O)c2cc(Cl)sc2NC(=O)Cc2ccccc2)C(=O)N1]

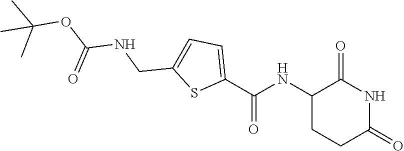 [SMILES: CC(C)(C)OC(=O)NCc1ccc(C(=O)NC2CCC(=O)NC2=O)s1]

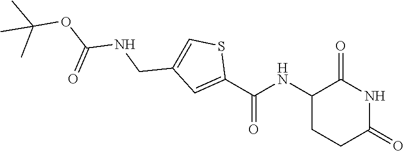 [SMILES: CC(C)(C)OC(=O)NCc1csc(C(=O)NC2CCC(=O)NC2=O)c1]

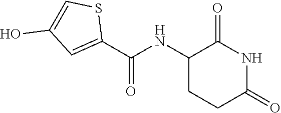 [SMILES: O=C1CCC(NC(=O)c2cc(O)cs2)C(=O)N1]

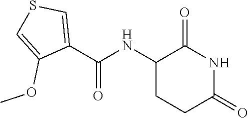 [SMILES: COc1cscc1C(=O)NC1CCC(=O)NC1=O]

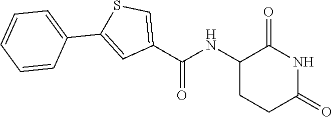 [SMILES: O=C1CCC(NC(=O)c2csc(-c3ccccc3)c2)C(=O)N1]

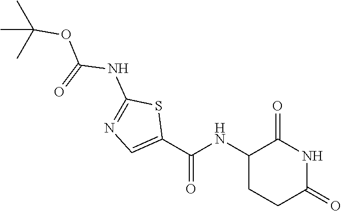 [SMILES: CC(C)(C)OC(=O)Nc1ncc(C(=O)NC2CCC(=O)NC2=O)s1]

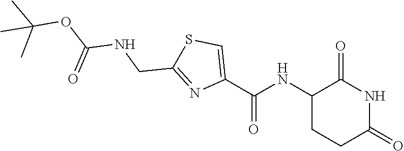 [SMILES: CC(C)(C)OC(=O)NCc1nc(C(=O)NC2CCC(=O)NC2=O)cs1]

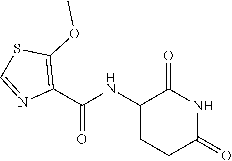 [SMILES: COc1scnc1C(=O)NC1CCC(=O)NC1=O]

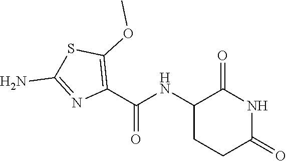 [SMILES: COc1sc(N)nc1C(=O)NC1CCC(=O)NC1=O]

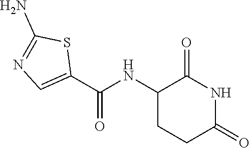 [SMILES: Nc1ncc(C(=O)NC2CCC(=O)NC2=O)s1]

Example ligase ligand moieties of Formula (IIa) and Formula (IIb) are shown in Table 7 below. Many of these compounds could be modified to allow attachment to the [linker](e.g. by C—H bond activation).

TABLE 7

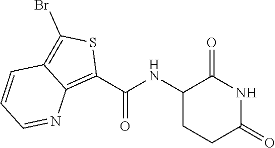 [SMILES: O=C1CCC(NC(=O)c2sc(Br)c3cccnc23)C(=O)N1]

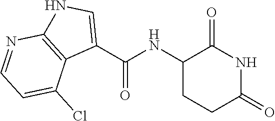 [SMILES: O=C1CCC(NC(=O)c2c[nH]c3nccc(Cl)c23)C(=O)N1]

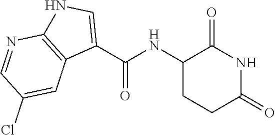 [SMILES: O=C1CCC(NC(=O)c2c[nH]c3ncc(Cl)cc23)C(=O)N1]

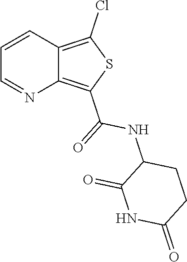 [SMILES: O=C1CCC(NC(=O)c2sc(Cl)c3cccnc23)C(=O)N1]

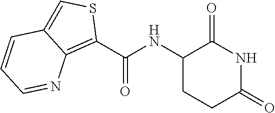 [SMILES: O=C1CCC(NC(=O)c2scc3cccnc23)C(=O)N1]

### Ligase Ligand Moieties of Formula (VIa) and Formula (VIb)

In the fourth, eighth and tenth aspects of the invention, the ligase ligand moiety may be of Formula (VIa) or (VIb):

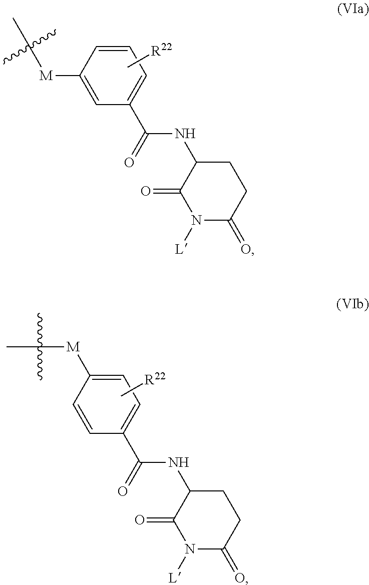

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl.

In some embodiments, L′ is hydrogen. In some embodiments, M is O. In some embodiments, R²² is unsubstituted C₁-C₆ alkyl.

The synthesis of the ligase ligand moieties of Formula (VIa) and (VIb) is set out in the Examples section, below.

Example ligase ligand moieties of Formula (VIa) and Formula (VIb) are shown in Table 8 below, where [symbol] indicates attachment to R¹⁸ of the linker:

TABLE 8

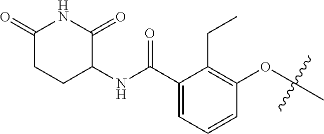 [SMILES: *Oc1cccc(C(=O)NC2CCC(=O)NC2=O)c1CC]

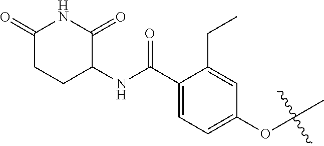 [SMILES: *Oc1ccc(C(=O)NC2CCC(=O)NC2=O)c(CC)c1]

### Linkers

In the first, second, third, fourth, ninth and tenth aspects of the present invention, the [linker] has the formula R¹⁴-R¹⁵-R¹⁶-R¹⁷-R¹⁸
wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—C₁₋₆ alkyl-, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl, —C₁₋₆ alkyl-C(O)—, cycloalkyl, CH₂—NH—C(O)—, heterocycloalkyl, or is absent wherein at least one of R¹⁴-R¹⁸ is present.

In some embodiments of the first, second, third, fourth, ninth and tenth aspects of the present invention, R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent; R¹⁷ is —CH₂(C₂H₄—O)_{y}, —(C₂H₄—O)ₓ, —(C₃H₆—O)ₓ, or is absent; and R¹⁸ is —C₁₋₆ alkyl, cycloalkyl, —CH₂—NH—C(O)—, heterocycloalkyl, or is absent

In some embodiments of the first, second, third, fourth, ninth and tenth aspects of the present invention, R¹⁴ is —C₁₋₆ alkyl, —C₂₋₆ alkenyl, —C₂₋₆ alkynyl, —C(O)—, —SO₂— or is absent. In some such embodiments, R¹⁴ is —C₁₋₆ alkyl.

In some embodiments of the first, second, third, fourth, ninth and tenth aspects of the present invention, R¹⁵ is cycloalkyl, heterocycloalkyl, aryl, heteroaryl, —C₁₋₆ alkyl-NH—, -cycloalkyl-NH— or is absent. In some such embodiments, R¹⁵ is heterocycloalkyl or is absent. In some such embodiments, R¹⁵ is piperazine, bridged piperazine, piperazine N-oxide, piperazine cation,

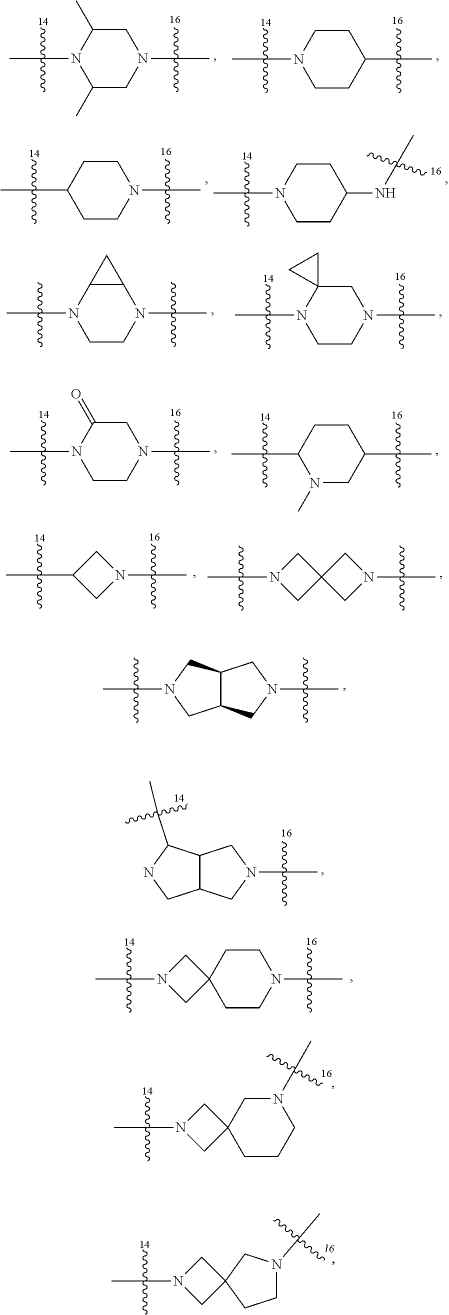

or is absent;
wherein [symbol] indicates attachment to R¹⁴ and [symbol] indicates attachment to R¹⁶.

In some embodiments of the first, second, third, fourth, ninth and tenth aspects of the present invention, R¹⁵ is piperazine,

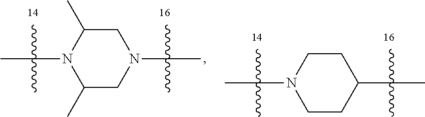

or is absent.

In some embodiments of the first, second, third, fourth, ninth and tenth aspects of the present invention, R¹⁶ is —C₁₋₆ alkyl, —CH₂—C(O)—NH—, —CH₂—C(O)—, —C(O)— or is absent.

In some embodiments of the first, second, third, fourth, ninth and tenth aspects of the present invention, R¹⁷ is absent.

In some embodiments of the first, second, third, fourth, ninth and tenth aspects of the present invention, R¹⁸ is —C₁₋₆ alkyl, cyclobutyl, CH₂—NH—C(O)—, piperazine or is absent.

In some embodiments of the first, second, third, fourth, ninth and tenth aspects of the present invention,
R¹⁴ is —C₁₋₆ alkyl, —C₁₋₆ alkyl-N(Me)-, —SO₂— or is absent;
R¹⁵ is piperazine, bridged piperazine, piperazine N-oxide, piperazine cation,

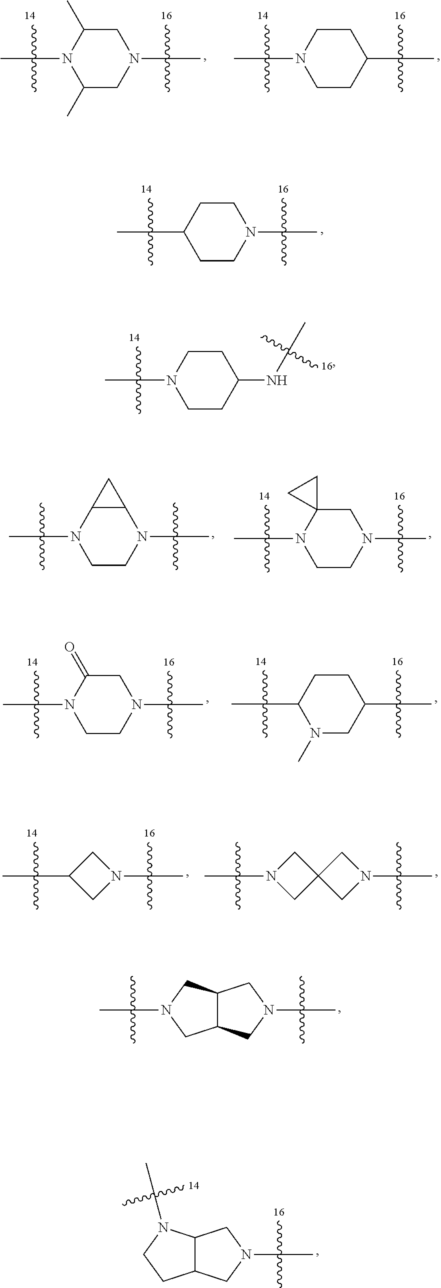

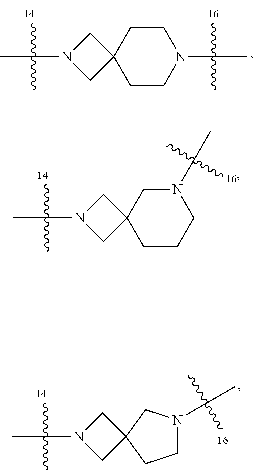 [SMILES: *N1CCC2(CC1)CN(*)C2 ; *N1CCCC2(C1)CN(*)C2 ; *N1CCC2(C1)CN(*)C2]

or is absent,
wherein [symbol] in attachment to R¹⁴ and [symbol] indicates attachment to R¹⁶
R¹⁶ is —C₆alkyl, —CH₂—C(O)—NH—, —CH₂—C(O)—, —C(O)— or is absent;
R¹⁷ is —CH₂(C₂H₄—O)_{y}, (C₂H₄—O)ₓ, (C₃H₆—O)ₓ, or is absent, wherein x is 1-6 and y is 2-6; and
R¹⁸ is —C₁-6 alkyl, piperazine,

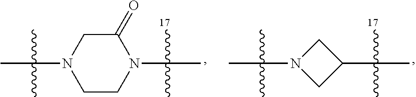 [SMILES: *N1CCN(*)C(=O)C1 ; *C1CN(*)C1]

cyclobutyl, —CH₂—NH—C(O)— or is absent, wherein [symbol] indicates attachment to R¹⁷
and wherein at least one of R¹⁴-R¹⁸ is present.

In some embodiments of the first, second, third, fourth, ninth and tenth aspects of the present invention,
R¹⁴ is —C₁₋₆ alkyl,
R¹⁵ is piperazine,

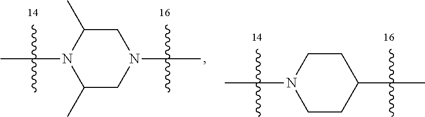

or is absent,
R¹⁶ is —C₁₋₆ alkyl, —CH₂—C(O)—NH—, —CH₂—C(O)—, —C(O)— or is absent
R¹⁷ is absent
R¹⁸ is —C₁₋₆ alkyl, cyclobutyl, CH₂—NH—C(O)—, piperazine or is absent.

In some embodiments of the first, second, third, fourth, ninth and tenth aspects of the present invention, [linker] is selected from

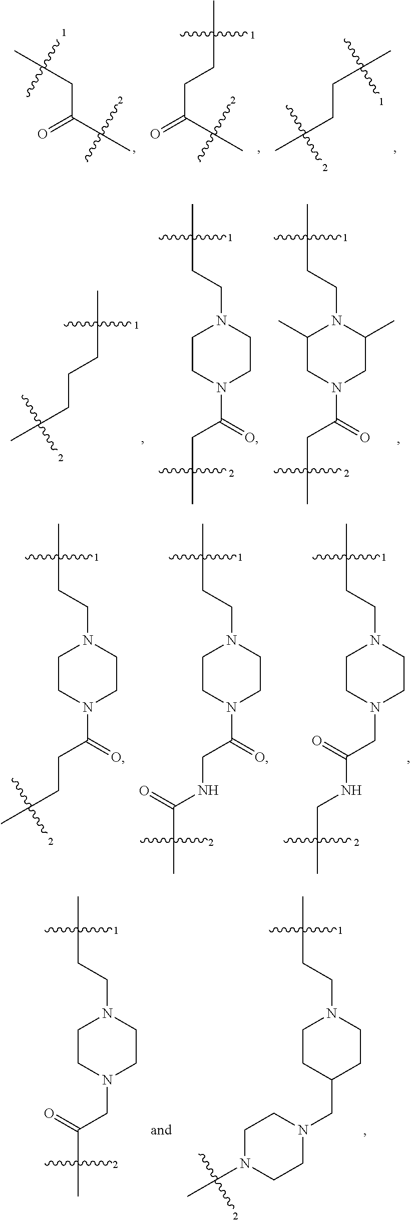

wherein
   [symbol] indicates attachment to [MCI-1 ligand moiety] and
   [symbol] indicates attachment to [ligase ligand moiety].

In some embodiments of the first, second, third, fourth, ninth and tenth aspects of the present invention, [linker] is selected from

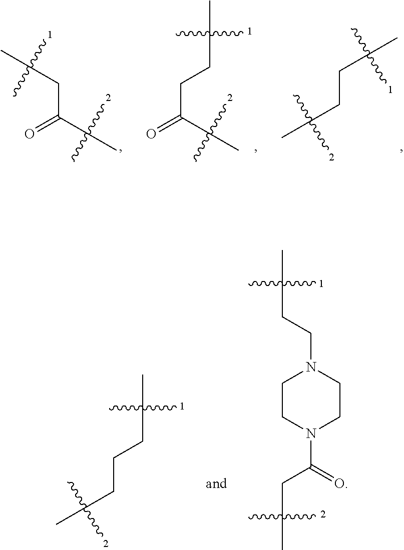

In some embodiments of the first, second, third, fourth, ninth and tenth aspects of the present invention, [linker] is selected from

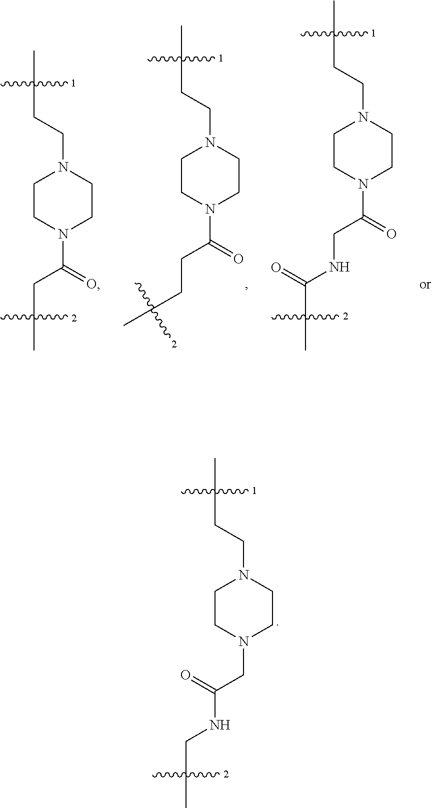

In the fifth, sixth, seventh and eighth aspects of the present invention, the [linker] has the formula

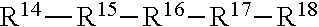

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, C(O)—C₁₋₆ alkyl-, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(CH₂O)ₓ, (C₂H₄—O)ₓ, (C₃H₆—O)ₓ, or is absent
   x is 1-10
   y is 2-10
   R¹⁸ is —C₁₋₆ alkyl-C(O)—, cycloalkyl or CH₂—NH—C(O)—.

In some embodiments of the fifth, sixth, seventh and eighth aspects of the present invention, R¹⁶ is —C₁₋₆ alkyl, —C(O)—, C(O)—NH—, —C(O)O—, —CH₂—C(O)—, —CH₂—C(O)—NH—, —CH₂—C(O)O— or is absent; R¹⁷ is —CH₂(C₂H₄—O)_{y}, (C₂H₄—O)ₓ, (C₃H₆—O)ₓ, or is absent; and R¹⁸ is cycloalkyl or CH₂—NH—C(O)—.

In some embodiments of the fifth, sixth, seventh and eighth aspects of the present invention, R¹⁴ is —C₁₋₆ alkyl, —C₂₋₆ alkenyl, —C₂₋₆ alkynyl, —C(O)—, —SO₂— or is absent. In some such embodiments, R¹⁴ is —C₁₋₆ alkyl.

In some embodiments of the fifth, sixth, seventh and eighth aspects of the present invention, R¹⁵ is cycloalkyl, heterocycloalkyl, aryl, heteroaryl, —C₁₋₆ alkyl-NH—, -cycloalkyl-NH— or is absent. In some such embodiments, R¹⁵ is heterocycloalkyl or is absent. In some embodiments, R¹⁵ is heterocycloalkyl. In some embodiments, R¹⁵ is piperazine, bridged piperazine, piperazine N-oxide, piperazine cation,

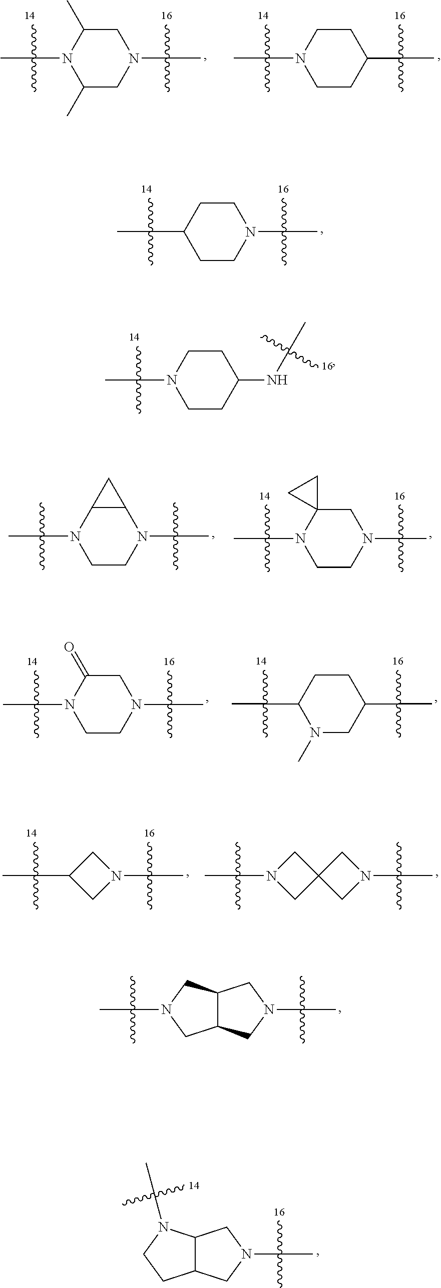

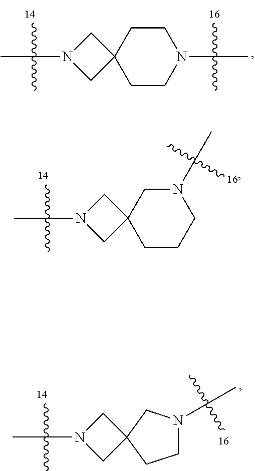 [SMILES: *N1CCC2(CC1)CN(*)C2 ; *N1CCCC2(C1)CN(*)C2 ; *N1CCC2(C1)CN(*)C2]

or is absent;
wherein [symbol] indicates attachment to R¹⁴ and [symbol] indicates attachment to R¹⁶.

In some embodiments of the fifth, sixth, seventh and eighth aspects of the present invention, R⁵ is piperazine,

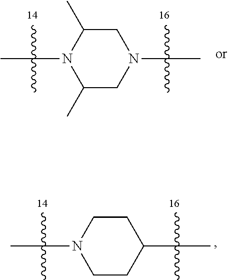

wherein [symbol] indicates attachment to R¹⁴ and [symbol] indicates attachment to R¹⁶. In some embodiments, R¹⁵ is piperazine.

In some embodiments of the fifth, sixth, seventh and eighth aspects of the present invention, R¹⁶ is —C₁₋₆ alkyl, —CH₂—C(O)—NH—, —CH₂—C(O)—, —C(O)— or is absent. In some embodiments, R¹⁶ is —C₁₋₆ alkyl, —CH₂—C(O)— or —C(O)—. In some embodiments, R¹⁶ is —C(O)—.

In some embodiments of the fifth, sixth, seventh and eighth aspects of the present invention, R¹⁷ is absent.

In some embodiments of the fifth, sixth, seventh and eighth aspects of the present invention, R¹⁸ is cycloalkyl. In some such embodiments, R¹⁸ is cyclobutyl. In other embodiments, R¹⁸ is or —CH₂—NH—C(O)—.

In other embodiments of the fifth, sixth, seventh and eighth aspects of the present invention, [linker] is

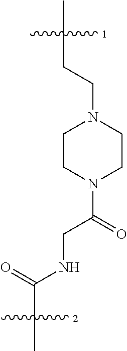 [SMILES: *CCN1CCN(C(=O)CNC(*)=O)CC1]

wherein
   [symbol] indicates attachment to [MCL-1 ligand moiety] and
   [symbol] indicates attachment to [ligase ligand moiety].

Linkers as used in the compounds of the present invention may be synthesized according to standard methods.

Most of the alkyl and polyethylene glycol (PEG) linkers were commercially available, or prepared due to procedures described in literature.

Examples of commercially available linkers include:

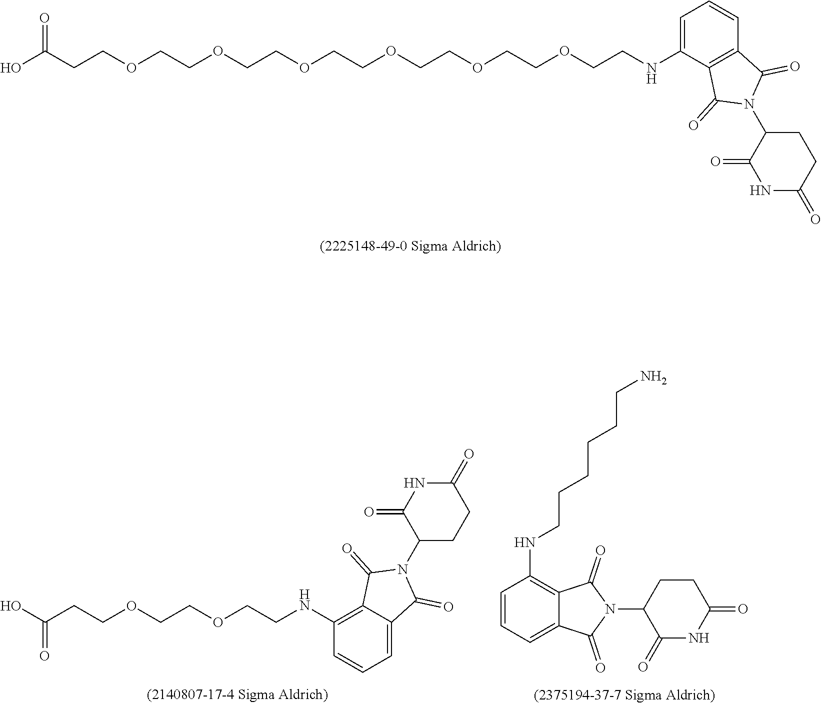 [SMILES: O=C(O)CCOCCOCCOCCOCCOCCOCCNc1cccc2c1C(=O)N(C1CCC(=O)NC1=O)C2=O ; O=C(O)CCOCCOCCNc1cccc2c1C(=O)N(C1CCC(=O)NC1=O)C2=O ; NCCCCCCNc1cccc2c1C(=O)N(C1CCC(=O)NC1=O)C2=O]

Synthesis of linkers which are not commercially available are described in the examples.

Linkers containing modifications of piperazine were prepared according to the following scheme:

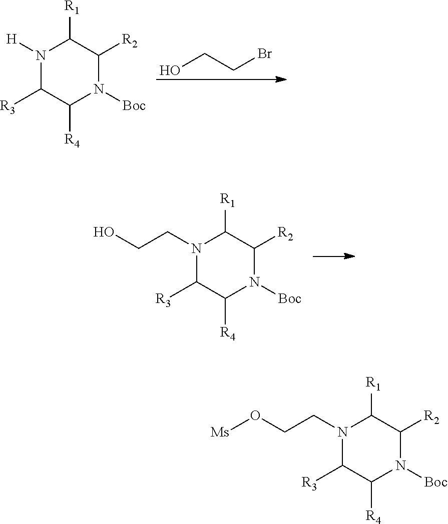

### MCL-1 Ligand Moieties

In the first, second, third and fourth aspects of the present invention, the [MCL-1 ligand moiety] is a compound of Formula (A1), (A2), (A3) or (A4):

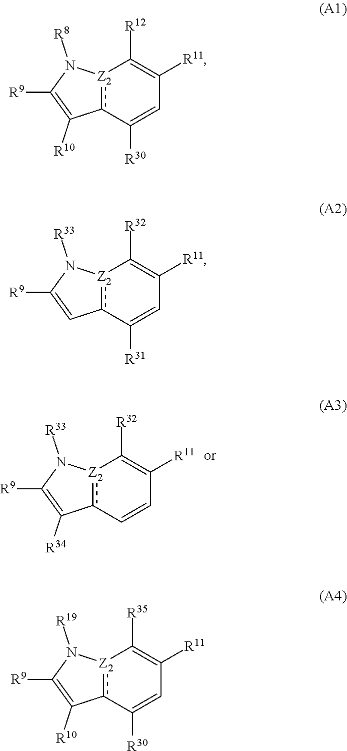

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,

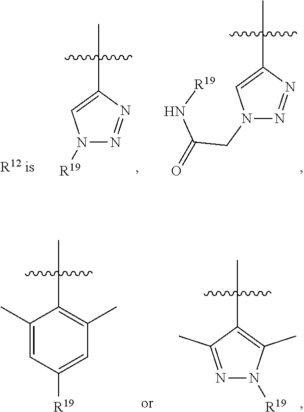

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, R

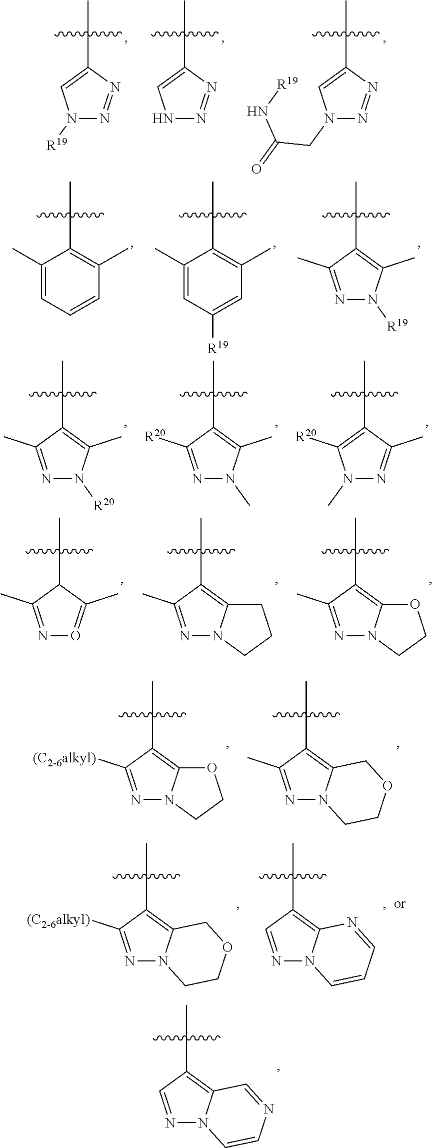

wherein R²⁰ is Me, —CH₂—OMe, —(CH₂CH₂O)ₚ(C₁-C₆ alkyl) or —CH₂—O-bromobenzaldehyde, wherein p is 1-5;
   R³⁴ is C₂₋₅alkyl-O—R¹³ or —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
   R³⁵ is or

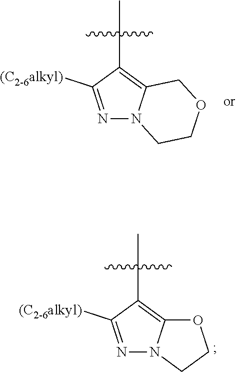

and
R¹⁹ is a bond connected to R¹⁴ of the linker, wherein each of Formula (A1), Formula (A2), Formula (A3) and Formula (A4) contains a single R¹⁹.

In some embodiments of the first, second, third and fourth aspects of the present invention, [MCL-1 ligand moiety] is of Formula (A1):

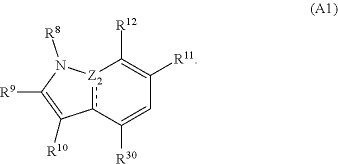

In other embodiments of the first, second, third and fourth aspects of the present invention, [MCL-1 ligand moiety] is of Formula (A2):

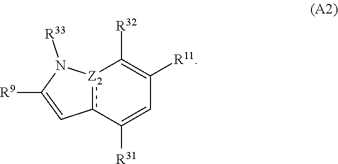

In other embodiments of the first, second, third and fourth aspects of the present invention, [MCL-1 ligand moiety] is of Formula (A3):

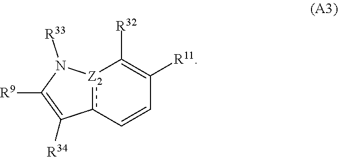

In other embodiments of the first, second, third and fourth aspects of the present invention, [MCL-1 ligand moiety] is of Formula (A4):

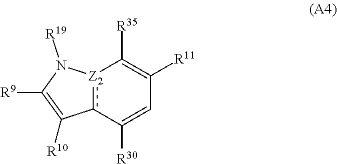

In some embodiments of the first, second, third and fourth aspects of the present invention, in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments of the first, second, third and fourth aspects of the present invention, in Formula (A1), R² is

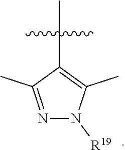

In some embodiments of the first, second, third and fourth aspects of the present invention, in Formula (A4), R³⁵ is

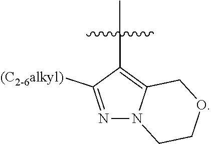

In some embodiments of the first, second, third and fourth aspects of the present invention, in Formula (A2), R³¹ is —C₂₋₅alkyl-O—R¹³ or —O—C₂₋₅alkyl-R³, wherein R¹³ is phenyl or naphthyl, wherein the phenyl or naphthyl is optionally substituted with at least one substituent selected from halogen and C₁-C₆ alkyl.

In some embodiments of the first, second, third and fourth aspects of the present invention, in Formula (A3), R³⁴ is —O—C₂₋₅alkyl-R³, wherein R¹³ is phenyl or naphthyl, wherein the phenyl or naphthyl is optionally substituted with at least one substituent selected from halogen and C₁-C₆ alkyl.

In some embodiments of the first, second, third and fourth aspects of the present invention, Z₂ is C and [symbol] is a double bond.

In some embodiments of the first, second, third and fourth aspects of the present invention, each R⁹ is independently —C(O)OH or —P(O)(OH)₂.

In some embodiments of the first, second, third and fourth aspects of the present invention, R¹¹ is halogen.

In some embodiments of the first, second, third and fourth aspects of the present invention, each R²⁰ is Me.

In some embodiments of the first, second, third and fourth aspects of the present invention, [MCL-1 ligand moiety] is selected from:

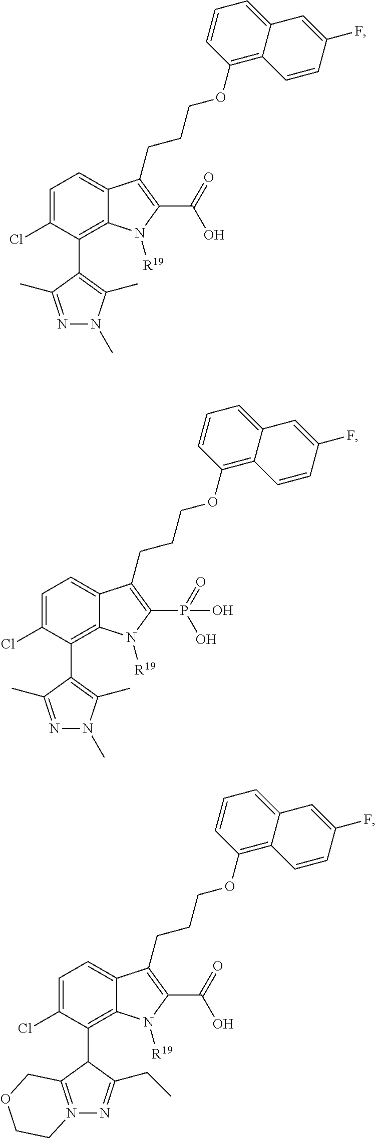

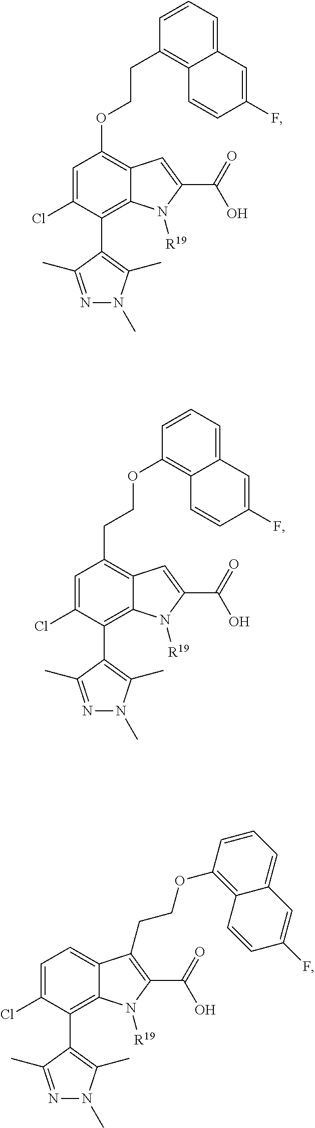

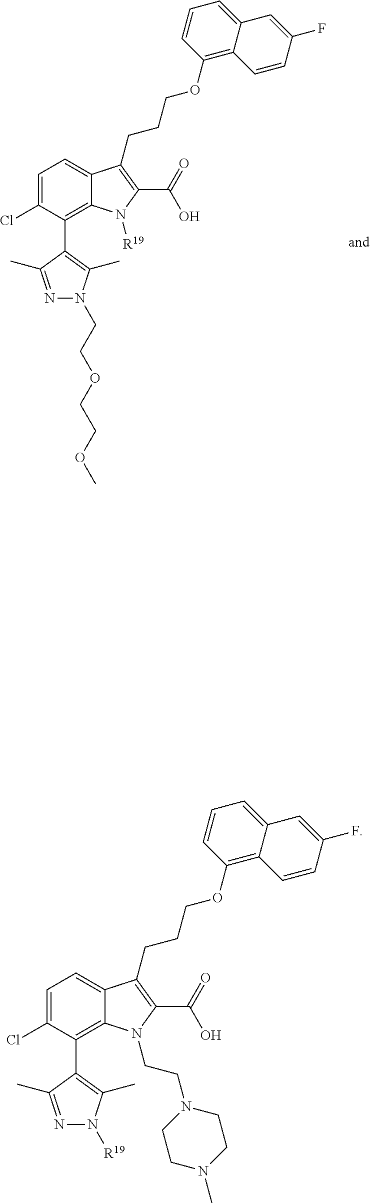

In the fifth, sixth, seventh, eighth, ninth and tenth aspects of the present invention, [MCL-1 ligand moiety] is a compound of Formula (A):

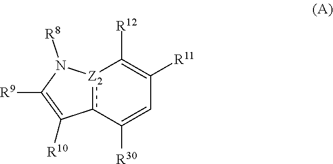

wherein
   [symbol] is a single bond or a double bond;
   R⁸ is H, R¹⁹, or C₁-C₅ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

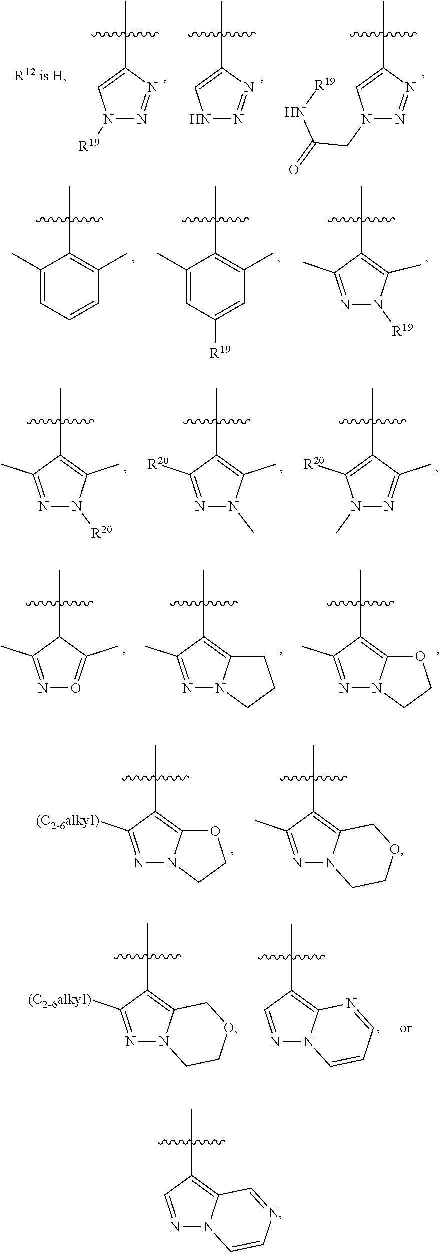

wherein R²⁰ is Me, —CH₂—OMe, —(CH₂CH₂O)ₚ(C₁-C₆ alkyl), —CH₂—O-bromobenzaldehyde, or

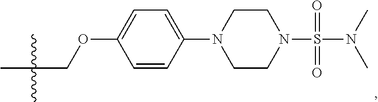

wherein p is 1-5; or
when R¹² is

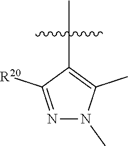

and R¹⁰ is —O-naphthyl substituted with —O— or —S—, then R²⁰ is

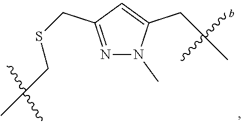 [SMILES: *CSCc1cc(C*)n(C)n1]

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹.

In some embodiments of the fifth, sixth, seventh, eighth, ninth and tenth aspects of the present invention, one of R¹⁰ and R³⁰ is H and the other of R¹⁰ and R³⁰ is —C₂₋₅alkyl-O—R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

In some embodiments of the fifth, sixth, seventh, eighth, ninth and tenth aspects of the present invention, R³⁰ is H.

In some embodiments of the fifth, sixth, seventh, eighth, ninth and tenth aspects of the present invention, R¹³ is phenyl or naphthyl, wherein the phenyl or naphthyl is optionally substituted with at least one substituent selected from halogen and C₁-C₆ alkyl.

In some embodiments of the fifth, sixth, seventh, eighth, ninth and tenth aspects of the present invention, R¹² is H,

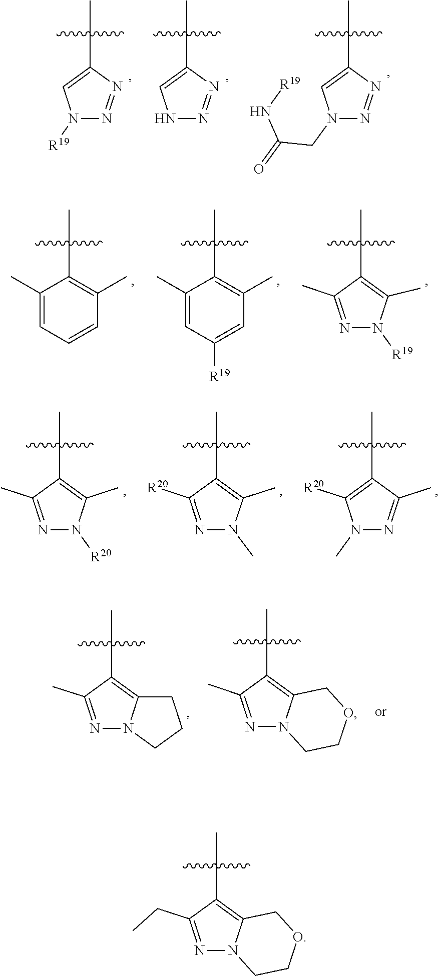

In some embodiments of the fifth, sixth, seventh, eighth, ninth and tenth aspects of the present invention, R¹² is

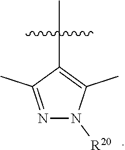

In some such embodiments, R²⁰ is Me.

In some embodiments of the fifth, sixth, seventh, eighth, ninth and tenth aspects of the present invention R²⁰ is Me, —CH₂—O-bromobenzaldehyde, or

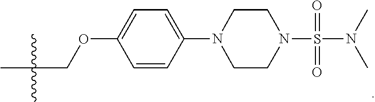 [SMILES: *COc1ccc(N2CCN(S(=O)(=O)N(C)C)CC2)cc1]

In some embodiments of the fifth, sixth, seventh, eighth, ninth and tenth aspects of the present invention, Z₂ is C and [symbol] is a double bond.

In some embodiments of the fifth, sixth, seventh, eighth, ninth and tenth aspects of the present invention, R¹¹ is hydrogen. In other embodiments, R¹¹ is halogen. In some such embodiments, R¹¹ is Cl.

In some embodiments of the fifth, sixth, seventh, eighth, ninth and tenth aspects of the present invention, R⁹ is —C(O)OH.

In some embodiments of the fifth, sixth, seventh, eighth, ninth and tenth aspects of the present invention, [MCL-1 ligand moiety] is selected from:

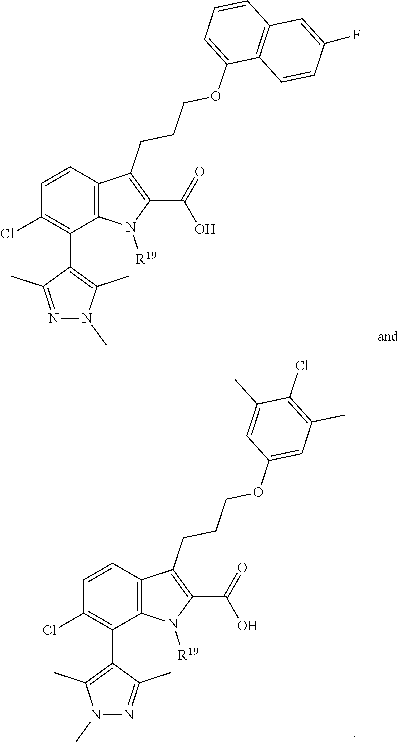

In some embodiments of the fifth, sixth, seventh, eighth, ninth and tenth aspects of the present invention, [MCL-1 ligand moiety] is:

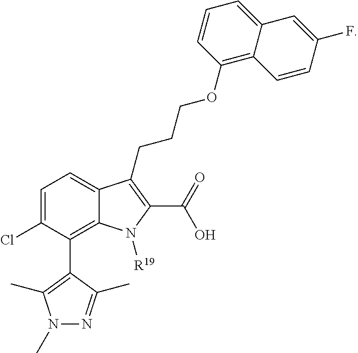

### EXAMPLES

There are a number of ways in which the bifunctional compounds [MCL-1 ligand moiety]-[linker]-[ligase ligand moiety] of the present application may be synthesized:

1. [Mcl-1 Ligand Moiety] is Coupled with linkerA, Followed by Coupling with [Ligase Ligand Moiety]-linkerB

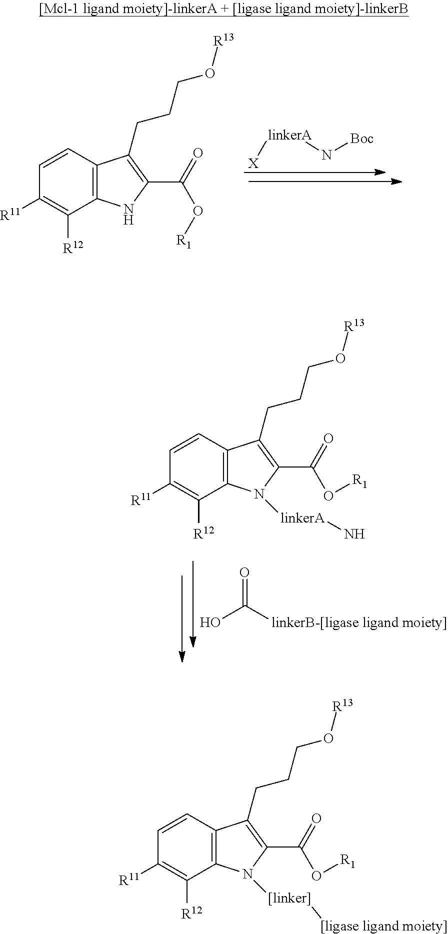

wherein X is halogen or OMs, OTs; linkerA-N-Boc corresponds to linkerA terminating with a Boc-protected primary or secondary amine; linkerA-NH corresponds to linkerA terminating with a primary or secondary amine; and R¹¹-R¹³ and [linker] are as defined herein; wherein [linker] is formed in the above synthesis by the reaction of -linkerA-NHR^{W} with -linkerB—COOH.

Examples of this method are set out below:

(a) Attachment of Linker Via R⁸ of [MCL-1 Ligand Moiety], and Coupling of linkerA and linkerB Via an Amide Bond:

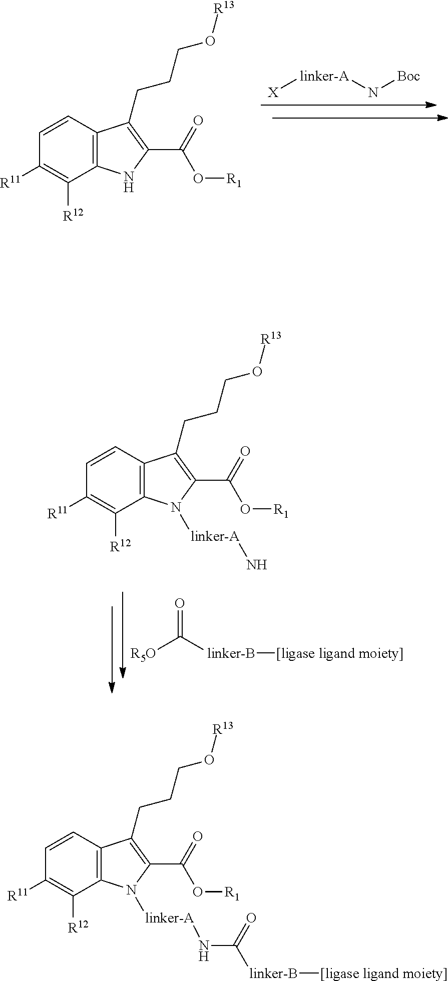

wherein X is halogen or OMs, OTs; linkerA-N-Boc corresponds to linkerA terminating with a Boc-protected primary or secondary amine; linkerA-NH corresponds to linkerA terminating with a primary or secondary amine; R₅ is succinimidyl or pentafluorophenyl; and R¹¹-R¹³ are as defined herein; and wherein linkerA-NHC(O)-linkerB corresponds to [linker].
   (b) Attachment of Linker Via R⁸ of [MCL-1 Ligand Moiety], and Coupling of linkerA and linkerB by Alkylation or Reductive Amination

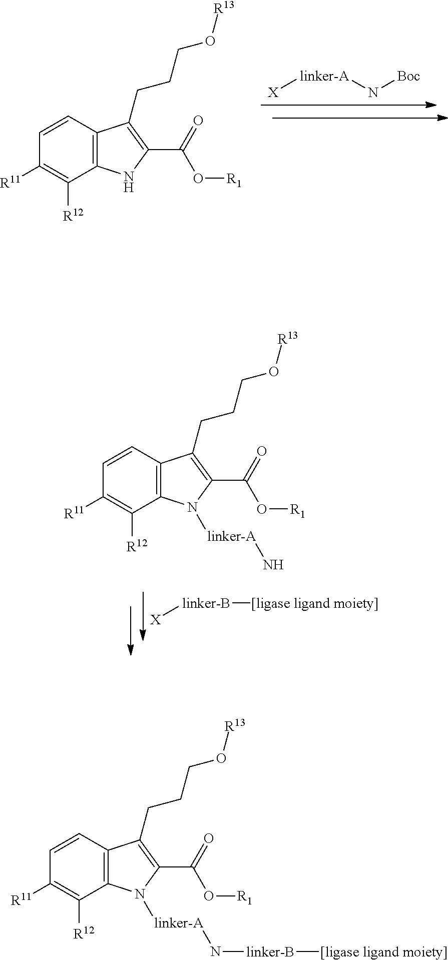

wherein X is halogen or OMs, OTs; linkerA-N-Boc corresponds to linkerA terminating with a Boc-protected primary or secondary amine; linkerA-NH corresponds to linkerA terminating with a primary or secondary amine; and R¹¹-R¹³ are as defined herein; and wherein linkerA-N-linkerB corresponds to [linker].
   2. [Mcl-1 Ligand Moiety] is Coupled with linkerA, Followed by Coupling with linkerB, Followed Coupling with [Ligase Ligand Moiety]

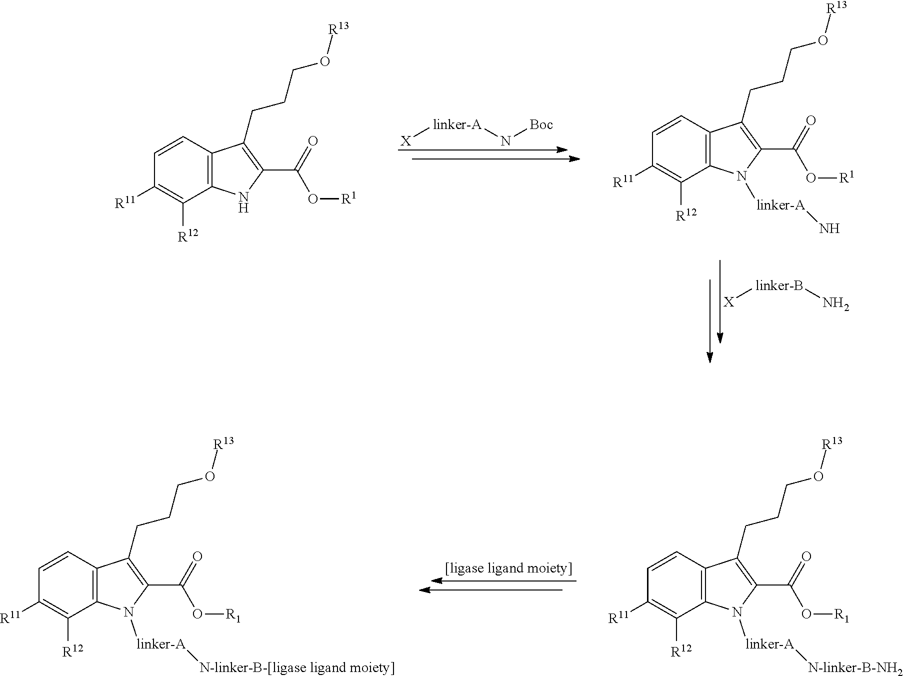

wherein X is halogen or OMs, OTs; linkerA-N-Boc corresponds to linkerA terminating with a Boc-protected primary or secondary amine; linkerA-NH corresponds to linkerA terminating with a primary or secondary amine; and R¹¹-R¹³ are as defined herein; and wherein linkerA-N-linkerB corresponds to [linker].
   3. [Mcl-1 Ligand Moiety]-[Linker] is Coupled with [Ligase Ligand Moiety]

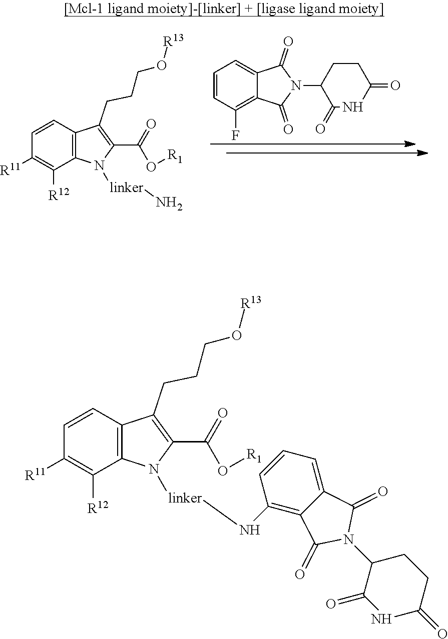

4. [Mcl-1 Ligand Moiety] is Coupled with [Linker]-[Ligase Ligand Moiety]

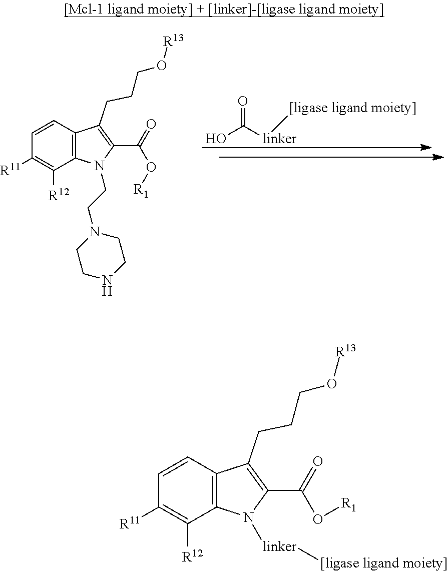

5. [Mcl-1 Ligand Moiety]-[Linker]-R^{v} is Coupled with 3-aminopiperidine-2,6-dione

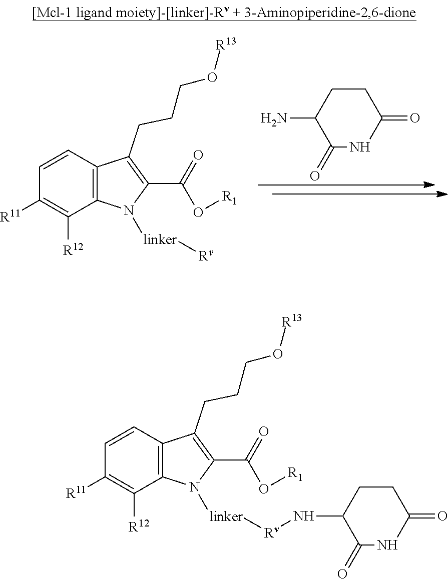

wherein R¹¹-R¹³ are as defined herein; and R^{v} is -T-R^{x}, -T-R^{y},

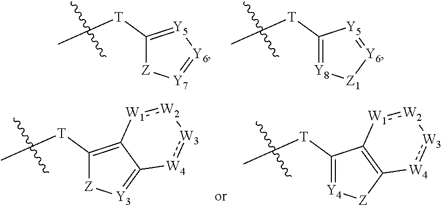

The bifunctional compounds of the present invention were prepared as follows:

### Example 1: 6-Chloro-1-(2-(4-(2-(4-((2,6-dioxopiperidin-3-yl)carbamoyl)-7-fluoro-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 1)

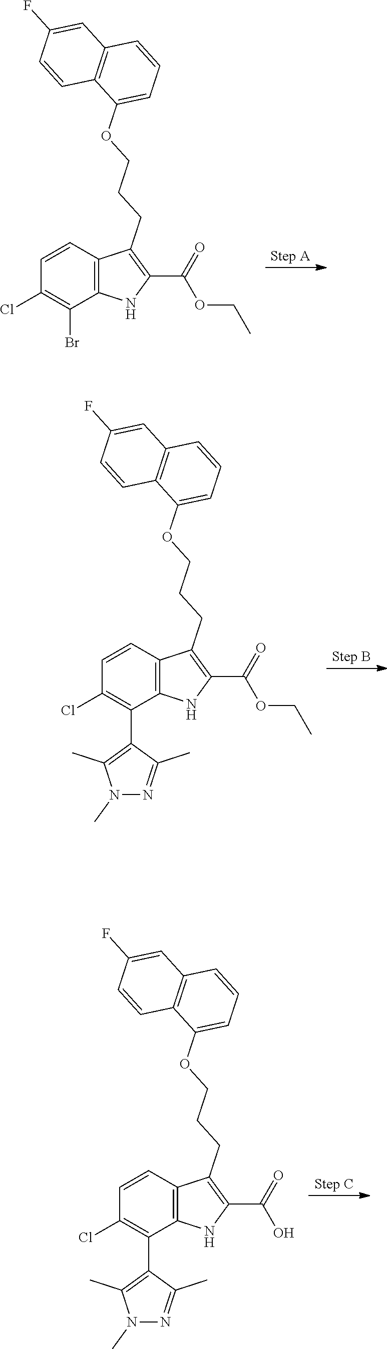

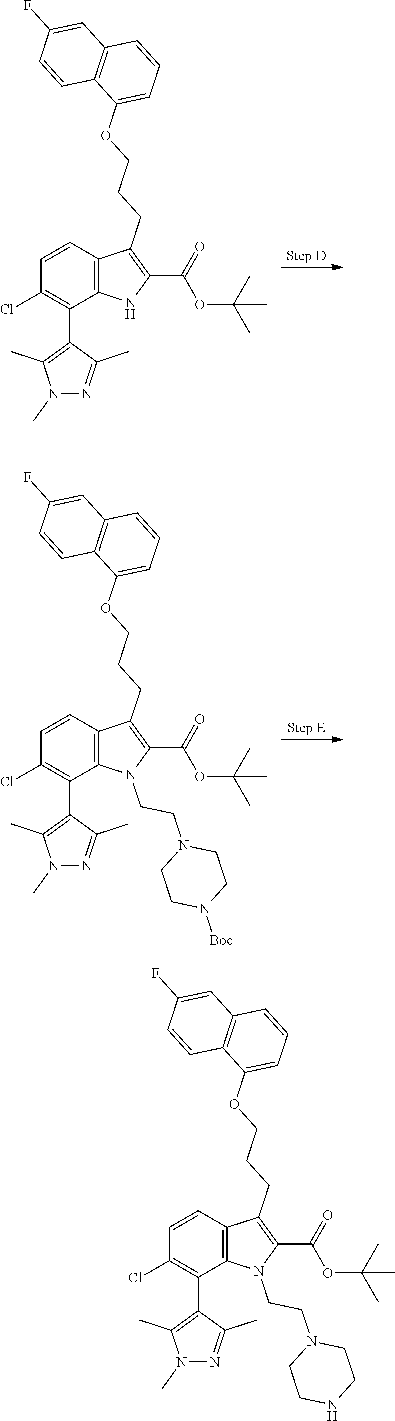

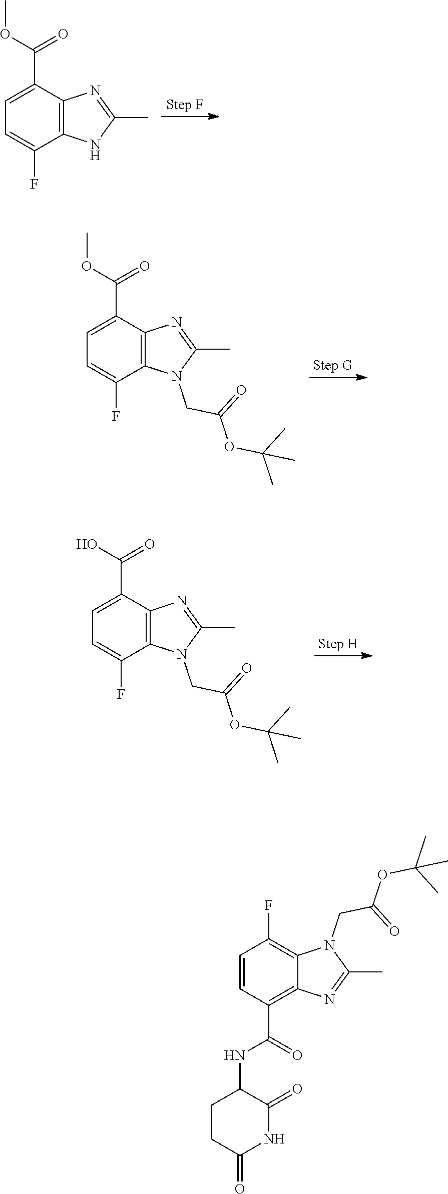 [SMILES: COC(=O)c1ccc(F)c2[nH]c(C)nc12 ; COC(=O)c1ccc(F)c2c1nc(C)n2CC(=O)OC(C)(C)C ; Cc1nc2c(C(=O)O)ccc(F)c2n1CC(=O)OC(C)(C)C ; Cc1nc2c(C(=O)NC3CCC(=O)NC3=O)ccc(F)c2n1CC(=O)OC(C)(C)C]

### Step A

To a stirred solution of ethyl 7-bromo-6-chloro-3-(3-((6-fluoronaphthalen-1-yl)oxy)propyl)-1H-indole-2-carboxylate (1.7 g, 3.368 mmol) in dioxane (20 mL) and water (5 mL) were added 1,3,5-trimethyl-4-(4,4,5,5-tetramethyl-1,3,2-dioxaborolan-2-yl)-1H-pyrazole (2.386 g, 10.103 mmol) and K₂CO₃ (1.859 g, 13.471 mmol). The mixture was deoxygenated with argon and to it was added Pd(dppf)Cl₂ (0.369 g, 0.505 mmol) under argon atmosphere. Then the reaction mixture was heated under reflux for 16 h. After complete consumption of the starting material (monitored by TLC and LCMS) the reaction mixture was filtered through celite pad and the solvents were evaporated under reduced pressure get the crude material. It was then diluted with EtOAc, washed successively with water and brine, the organic layer was dried over Na₂SO₄ and evaporated under reduced pressure to get the crude compound, which was then purified by column chromatography (SiO₂, 50% EtOAc in hexane) to get ethyl 6-chloro-3-(3-((6-fluoronaphthalen-1-yl)oxy)propyl)-7-(1,3,5-trimethyl-1H-pyrazol-4-yl)-1H-indole-2-carboxylate (1.2 g, 2.247 mmol, 66.72%) as brown solid.

LCMS (ESI): 534.2 m/z [M+H]⁺

### Step B

Ethyl 6-chloro-3-(3-((6-fluoronaphthalen-1-yl)oxy)propyl)-7-(1,3,5-trimethyl-1H-pyrazol-4-yl)-1H-indole-2-carboxylate (1.2 g, 2.251 mmol) was dissolved in EtOH (20 mL) and solution of NaOH (0.315 g, 7.88 mmol) in water (10 mL) was added to it. The mixture was heated under reflux for 16 h. After complete consumption of the starting material (monitored by TLC and LCMS) the reaction mixture was cooled down to room temperature, solvents were evaporated under reduced pressure to get the crude reaction mixture. It was then diluted with water, washed with EtOAc. Aqueous layer was carefully acidified using 1M HCl to pH=3 and extracted with EtOAc (×3). The organic layer was dried over Na₂SO₄, filtered, and concentrated in vacuo to afford 6-chloro-3-(3-((6-fluoronaphthalen-1-yl)oxy)propyl)-7-(1,3,5-trimethyl-1H-pyrazol-4-yl)-1H-indole-2-carboxylic acid (0.9 g, crude) as brown liquid which was used for the next step without further purification.

LCMS (ESI): 506.3 m/z [M+H]⁺

### Step C

6-chloro-3-(3-((6-fluoronaphthalen-1-yl)oxy)propyl)-7-(1,3,5-trimethyl-1H-pyrazol-4-yl)-1H-indole-2-carboxylic acid (1.2 g, 2.376 mmol) was suspended in toluene (20 mL) and the mixture was heated to reflux under nitrogen. N,N-dimethylformamide di-tert-butyl acetal (4.547 mL, 19.01 mmol) was added drop-wise to the refluxing mixture. The mixture was heated under reflux for 16 h under nitrogen. After complete consumption of the starting material (monitored by TLC and LCMS) the reaction mixture was then diluted with EtOAc, washed successively with sodium bicarbonate (aqueous, saturated), water and brine. Organic layer was dried over Na₂SO₄ and evaporated under reduced pressure to get the crude compound, which was then purified by column chromatography (SiO₂, 70% EtOAc in hexane) to get tert-butyl 6-chloro-3-(3-((6-fluoronaphthalen-1-yl)oxy)propyl)-7-(1,3,5-trimethyl-1H-pyrazol-4-yl)-1H-indole-2-carboxylate (0.772 g, 1.373 mmol, 61% over two steps) as brownish liquid.

LCMS (ESI): 561.9 m/z [M+H]⁺

### Step D

To solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (0.555 g, 0.989 mmol) in DMF (10 mL) was added tert-butyl 4-(2-chloroethyl)piperazine-1-carboxylate (0.492 g, 1.979 mmol) followed by cesium carbonate (1.607 g, 4.947 mmol) in DMF (5 mL) and the mixture was allowed to stir at 90° C. for 16 h under nitrogen. After complete consumption of the starting material (monitored by TLC and LCMS) the reaction mixture was diluted with EtOAc, washed successively with water and brine. The organic layer was dried over Na₂SO₄ and evaporated under reduced pressure to get the crude compound, which was then purified by column chromatography (SiO₂, 30% EtOAc in hexane) to get tert-butyl 1-(2-(4-(tert-butoxycarbonyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (0.5 g, 0.645 mmol, 65.27%) as off white solid.

LCMS (ESI): 774.6 m/z [M+H]⁺

### Step E

tert-butyl 1-(2-(4-(tert-butoxycarbonyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (0.3 g, 0.388 mmol) dissolved in 20 mL of 4M HCl in dioxan at 0° C. and the mixture was stirred for 2 h under nitrogen at the same temperature. After complete consumption of the starting material (monitored by TLC and LCMS) the reaction mixture was poured in to cold 1M NaOH solution and extracted several times with DCM. The combined organics were dried over Na₂SO₄ and concentrated in vacuo to get the crude compound which was then purified by column chromatography (amine SiO₂, 10% MeOH in DCM) to get tert-butyl 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (0.165 g, 0.244 mmol, 62.98%) as off white solid.

LCMS (ESI): 674.4 m/z [M+H]⁺

### Step F

To a solution of methyl 7-fluoro-2-methyl-1H-benzo[d]imidazole-4-carboxylate (400 mg, 1.923 mmol) in THF (15 mL) was added sodium hydride (60%, 69.23 mg, 2.885 mmol) portionwise at 0° C. under nitrogen, followed by tert-butyl bromoacetate (0.315 mL, 4.615 mmol) and the reaction mixture was allowed to stir for 2 h under nitrogen. After complete consumption of starting material (monitored by LC-MS and TLC), to the reaction mixture was added 5 mL of cold water to quench unreacted sodium hydride and then the reaction mixture was extracted with ethyl acetate. The combined organic layer was washed with water, saturated sodium chloride solution (20 mL), dried over anhydrous sodium sulphate and filtered. The filtrate was concentrated under reduced pressure to get the crude compound which was then purified by column chromatography (SiO₂, 60% EtOAc in DCM) to get methyl 1-(2-(tert-butoxy)-2-oxoethyl)-7-fluoro-2-methyl-1H-benzo[d]imidazole-4-carboxylate (450 mg, 1.39 mmol, 72%) of as brown solid.

LCMS (ESI): 322.5 m/z [M+H]⁺.

### Step G

To a stirred solution of methyl 1-(2-(tert-butoxy)-2-oxoethyl)-7-fluoro-2-methyl-1H-benzo[d]imidazole-4-carboxylate (200 mg, 0.621 mmol) in H₂O (1 mL) and MeCN (9 mL) was added LiBr (1618.32 mg, 18.63 mmol) followed by Et₃N (1.723 ml, 12.42 mmol) at room temperature. The mixture was stirred for 24 h at room temperature. After complete consumption of starting material (monitored by LCMS and TLC) the reaction mixture was concentrated under reduced pressure at low temperature. The residue was dissolved in water and washed with Et₂O. The aqueous layer was carefully acidified to pH=~7 using 1M HCl at 0° C. Product was extracted with EtOAc, washed with brine, dried over Na₂SO₄, filtered and dried under reduced pressure. Crude product was purified by triturating with Et₂O to afford 1-(2-(tert-butoxy)-2-oxoethyl)-7-fluoro-2-methyl-1H-benzo[d]imidazole-4-carboxylic acid (130 mg, 0.421 mmol, 68%) of as off white solid.

LCMS (ESI): 309.2 m/z [M+H]⁺

### Step H

1-(2-(tert-Butoxy)-2-oxoethyl)-7-fluoro-2-methyl-1H-benzo[d]imidazole-4-carboxylic acid (30.0 mg, 0.097 mmol), 3-aminopiperidine-2,6-dione hydrochloride (19.2 mg, 0.117 mmol) and HATU (74.0 mg, 0.195 mmol) were dissolved in dry DMF (1.2 mL) under argon atmosphere. To it, DIPEA (0.051 mL, 0.292 mmol) was added and the reaction (monitored with LCMS) was stirred for 15 min under argon at room temperature. After complete conversion of the starting material, the reaction was diluted with DMSO (3 mL). Crude product was purified with preparative HPLC (C18, H₂O:MeCN+0.1% FA) to give tert-butyl 2-{4-[(2,6-dioxopiperidin-3-yl)carbamoyl]-7-fluoro-2-methyl-1H-1,3-benzodiazol-1-yl}acetate (28.1 mg, 0.067 mmol, 69.0%) as white solid.

LCMS (ESI): 419.7 m/z [M+H]⁺

### Step I

tert-Butyl 2-{4-[(2,6-dioxopiperidin-3-yl)carbamoyl]-7-fluoro-2-methyl-1H-1,3-benzodiazol-1-yl}acetate (28.1 mg, 0.067 mmol) was dissolved in dry DCM under argon atmosphere and TFA (0.500 mL, 6.529 mmol) was added. Reaction, (monitored with LCMS), was stirred for 4 h at room temperature under argon. After complete consumption of the starting material reaction mixture was concentrated in vacuo. To the resulting residue a solution of aqueous 1M HCl was added (1 mL) and concentrated to dryness under reduced pressure. The addition of 1M HCl and evaporation sequence was repeated twice. Reaction product was dry-freezed to give 2-{4-[(2,6-dioxopiperidin-3-yl)carbamoyl]-7-fluoro-2-methyl-1H-1,3-benzodiazol-1-yl}acetic acid hydrochloride (25.8 mg, 0.065 mmol, 96.4%) as white solid.

LCMS (ESI): 363.0 m/z [M+H]⁺

### Step J

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and 2-{4-[(2,6-dioxopiperidin-3-yl)carbamoyl]-7-fluoro-2-methyl-1H-1,3-benzodiazol-1-yl}acetic acid hydrochloride (17.7 mg, 0.044 mmol) were dissolved in dry DMF (0.6 mL) under argon atmosphere and DIPEA (0.019 mL, 0.111 mmol) was added. To it, HATU (14.8 mg, 0.039 mmol) was added as a solution in DMF (0.6 mL). Reaction, (monitored with LCMS) was stirred at room temperature under argon for 25 min. After complete consumption of the starting material the solution was diluted with DCM up to 10 mL and washed with aqueous saturated solution of NaHCO₃ (10 mL) and brine (2×10 mL). Organic layer was dried over anhydrous magnesium sulphate, filtered and dried under reduced pressure to give crude tert-butyl 6-chloro-1-{2-[4-(2-{4-[(2,6-dioxopiperidin-3-yl)carbamoyl]-7-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (46.3 mg) as yellow oil, which was used in the next step without further purification.

LCMS (ESI): 1118.8 m/z [M+H]⁺

### Step K

Crude tert-butyl 6-chloro-1-{2-[4-(2-{4-[(2,6-dioxopiperidin-3-yl)carbamoyl]-7-fluoro-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (46.3 mg, 0.045 mmol) was dissolved under argon atmosphere in dry DCM (0.489 mL) and TFA (0.489 mL, 6.391 mmol) was added. Reaction (monitored with LCMS) was stirred at room temperature under argon for 16 h. After complete consumption of the starting material the mixture was concentrated to dryness under reduced pressure. The resulting residue was dissolved in DMSO and purified with preparative HPLC (C18, H₂O:MeCN+0.1% FA) to give 6-chloro-1-(2-(4-(2-(4-((2,6-dioxopiperidin-3-yl)carbamoyl)-7-fluoro-2-methyl-1H-benzo[d]imidazol-1-yl)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (18.7 mg, 0.019 mmol, 51.3% over 2 steps) as white solid.

LCMS (ESI): 962.20 m/z [M+H]⁺

¹H NMR (500 MHz, DMSO, 353 K) δ 10.58 (s, 1H), 9.98 (d, J=7.1 Hz, 1H), 8.25 (dd, J=9.2, 5.9 Hz, 1H), 7.85 (dd, J=8.5, 5.0 Hz, 1H), 7.72 (d, J=8.5 Hz, 1H), 7.58 (dd, J=10.4, 2.6 Hz, 1H), 7.46-7.39 (m, 2H), 7.32 (ddd, J=9.3, 8.6, 2.6 Hz, 1H), 7.23 (d, J=8.5 Hz, 1H), 7.07 (dd, J=11.3, 8.5 Hz, 1H), 6.88 (dd, J=5.4, 3.3 Hz, 1H), 5.25 (s, 2H), 4.85 (ddd, J=12.3, 7.2, 5.2 Hz, 1H), 4.37-4.15 (m, 4H), 3.78 (s, 3H), 3.48-3.36 (m, 4H), 3.32-3.27 (m, 2H), 2.80 (ddd, J=17.5, 13.1, 5.5 Hz, 1H), 2.65-2.56 (m, 1H), 2.52 (s, 3H), 2.35-2.29 (m, 1H), 2.29-2.22 (m, 2H), 2.22-2.06 (m, 7H), 2.03 (s, 3H), 1.90 (s, 3H).

### Example 2: 6-Chloro-1-(2-{4-[3-({4-[(2,6-dioxopiperidin-3-yl)carbamoyl]-2-methyl-1H-1,3-benzodiazol-6-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (Compound 3)

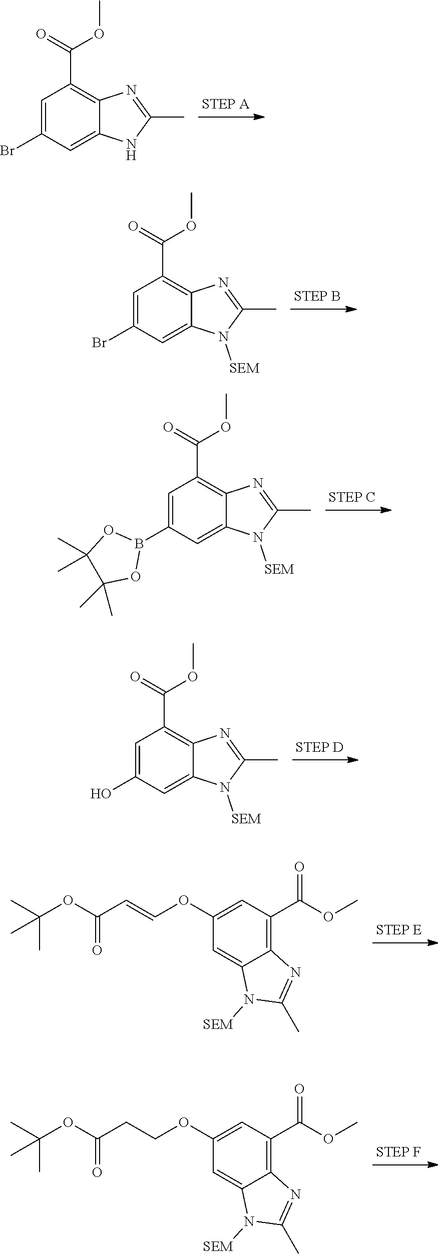

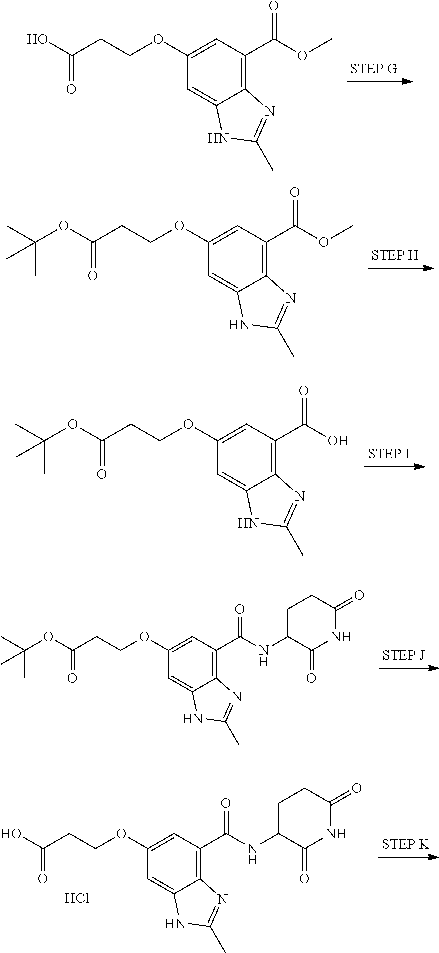

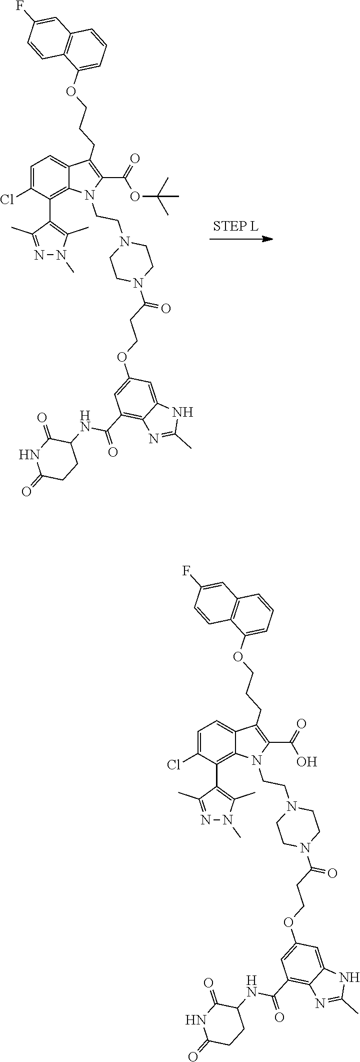

### Step A

To a stirred solution of methyl 6-bromo-2-methyl-1H-benzo[d]imidazole-4-carboxylate (2.5 g, 9.29 mmol) in DMF (50 mL) at 0° C. were added SEM chloride (3.29 mL, 18.6 mmol) and DIPEA (4.85 mL, 27.88 mmol) successively under nitrogen. The reaction mixture was allowed to stir at 80° C. for 16 h under nitrogen.

After complete consumption of starting material (checked by TLC), reaction was diluted with EtOAc and washed with cold water and brine successively and dried over Na₂SO₄ and evaporated under reduced pressure to get crude material which was purified by column chromatography (SiO₂, 50% EtOAc in DCM) to get methyl 6-bromo-2-methyl-1-((2-(trimethylsilyl)ethoxy)methyl)-1H-benzo[d]imidazole-4-carboxylate (2 g, 5.0 mmol, 54%) as yellowish gummy solid.

LCMS (ESI): 401.0 m/z [M+H]⁺

### Step B

To a well stirred solution of methyl 6-bromo-2-methyl-1-((2-(trimethylsilyl)ethoxy)methyl)-1H-benzo[d]imidazole-4-carboxylate (3 g, 7.5 mmol) in dioxane (50 mL) was added Pd(dppf)Cl₂ (0.550 g, 0.75 mmol) at RT under nitrogen. It was allowed to stir at 90° C. for 15 min. Then, the reaction mixture was cooled to RT. Bispinacolatodiborane (3.82 g, 15.04 mmol) and KOAc (1.476 g, 15.04 mmol) were added successively at RT under nitrogen. The resulting mixture was stirred at 90° C. for 16 h. After complete consumption of the starting material (monitored by TLC and LCMS), the reaction mixture was diluted with EtOAc, washed successively with water and brine solution. The organic layer was dried over Na₂SO₄ and evaporated under reduced pressure to get the crude compound which was purified by column chromatography (SiO₂, 40-50% EtOAc in DCM) to afford methyl 2-methyl-6-(4,4,5,5-tetramethyl-1,3,2-dioxaborolan-2-yl)-1-((2-(trimethylsilyl)ethoxy)methyl)-1H-benzo[d]imidazole-4-carboxylate (2.5 g, 5.6 mmol, 74%) as a yellowish liquid.

LCMS (ESI): 447.0 m/z [M+H]⁺

### Step C

To a stirred solution of methyl 2-methyl-6-(4,4,5,5-tetramethyl-1,3,2-dioxaborolan-2-yl)-1-((2-(trimethylsilyl)ethoxy)methyl)-1H-benzo[d]imidazole-4-carboxylate (4 g, 8.96 mmol) in MeOH (80 mL) and water (40 mL) was added mCPBA (1.697 g, 9.86 mmol) at 0° C. The resulting reaction mixture was stirred at RT for 6 h. After complete consumption of the starting material (monitored by TLC and LCMS), the reaction mixture was concentrated under reduced pressure, it was then diluted with EtOAc, washed successively with saturated NaHCO₃ solution, water and brine. The organic layer was dried over anhydrous Na₂SO₄ and evaporated under reduced pressure to get crude compound which was purified by column chromatography (SiO₂, 60% EtOAc in DCM) to get methyl 6-hydroxy-2-methyl-1-((2-(trimethylsilyl)ethoxy)methyl)-1H-benzo[d]imidazole-4-carboxylate (2.3 g, 6.84 mmol, 76%) as white solid.

LCMS (ESI): 337.0 m/z [M+H]⁺

### Step D

To a stirred solution of methyl 6-hydroxy-2-methyl-1-((2-(trimethylsilyl)ethoxy)methyl)-1H-benzo[d]imidazole-4-carboxylate (400 mg, 1.19 mmol) in DCM (15 mL) was added DMAP (436 mg, 3.57 mmol) and tert-butyl propiolate (0.326 mL, 2.381 mmol) successively at 0° C. under nitrogen. The resulting reaction mixture was stirred at RT for 1 h. After complete consumption of the starting material (monitored by TLC), the reaction was diluted with DCM and washed with water. The organic layer was dried over Na₂SO₄, filtered and concentrated under reduced pressure to get the crude compound which was purified by column chromatography (SiO₂, 30% EtOAc in DCM) to afford methyl (E)-6-((3-(tert-butoxy)-3-oxoprop-1-en-1-yl)oxy)-2-methyl-1-((2-(trimethylsilyl)ethoxy)methyl)-1H-benzo[d]imidazole-4-carboxylate (480 mg, 1.04 mmol, 87%) as a white solid.

LCMS (ESI): 463.0 m/z [M+H]⁺

### Step E

A stirring solution of methyl (E)-6-((3-(tert-butoxy)-3-oxoprop-1-en-1-yl)oxy)-2-methyl-1-((2-(trimethylsilyl)ethoxy)methyl)-1H-benzo[d]imidazole-4-carboxylate (450 mg, 0.97 mmol) in MeOH (10 mL) was degassed with argon for 15 minutes then Pd(OH)₂ (10% by weight, 450 mg) was added slowly. The reaction mixture was then stirred for 16 h at room temperature under H₂ balloon pressure (15 PSI). After complete consumption of the starting material, the reaction mixture was filtered through celite pad and the filtrate was concentrated under reduced pressure to get the crude material which was purified by column chromatography (SiO₂, 20%-30% EtOAc in DCM) to get methyl 6-(3-(tert-butoxy)-3-oxopropoxy)-2-methyl-1-((2-(trimethylsilyl)ethoxy)methyl)-1H-benzo[d]imidazole-4-carboxylate (180 mg, 0.387 mmol, 40%) as an off white solid.

LCMS (ESI): 465.0 m/z [M+H]⁺

### Step F

To a stirred suspension of methyl 6-(3-(tert-butoxy)-3-oxopropoxy)-2-methyl-1-((2-(trimethylsilyl)ethoxy)methyl)-1H-benzo[d]imidazole-4-carboxylate (1.1 g, 2.37 mmol) in DCM (6 mL) was added TFA (4 mL) drop wise at 0° C. under nitrogen. The mixture was allowed to stir at RT for 16 h. After complete consumption of the starting material (monitored by TLC and LCMS), the volatiles were evaporated under reduced pressure to get the crude compound (3-((4-(methoxycarbonyl)-2-methyl-1H-benzo[d]imidazol-6-yl)oxy)propanoic acid, (1 g of crude) which was then triturated with Et₂O and pentane and directly used for next step without further purification.

LCMS (ESI): 279.0 m/z [M+H]⁺

### Step G

(3-((4-(Methoxycarbonyl)-2-methyl-1H-benzo[d]imidazol-6-yl)oxy)propanoic acid (550 mg, crude) was dissolved in toluene (20 mL) and solution of 1,1-di-tert-butoxy-N,N-dimethylmethanamine (3.79 mL, 15.82 mmol) was added. The mixture was stirred at 110° C. for 3 hours. After complete consumption of the starting material (monitored by TLC and LCMS), the reaction mixture was diluted with EtOAc, washed successively with water and brine. The organic layer was dried over Na₂SO₄ and evaporated under reduced pressure to get the crude compound, which was then purified by column chromatography (SiO₂, 20%-30% EtOAc in hexane) to get methyl 6-(3-(tert-butoxy)-3-oxopropoxy)-2-methyl-1H-benzo[d]imidazole-4-carboxylate (150 mg, 0.449 mmol, 34% over 2 Steps) as white solid.

LCMS (ESI): 335.2 m/z [M+H]⁺

### Step H

To a solution of methyl 6-[3-(tert-butoxy)-3-oxopropoxy]-2-methyl-1H-1,3-benzodiazole-4-carboxylate (75.0 mg, 0.224 mmol) in H2O (1.0 mL) and MeCN (5.0 mL) was added LiBr (389.6 mg, 4.486 mmol) and Et₃N (0.313 mL, 2.243 mmol). The mixture was stirred for 3 days at RT. The crude was concentrated in vacuo and purified by reverse phase flash chromatography (C18, H₂O:MeCN+0.1% FA) to get 6-[3-(tert-butoxy)-3-oxopropoxy]-2-methyl-1H-1,3-benzodiazole-4-carboxylic acid (41.6 mg, 0.130 mmol, 57.9%) as white solid.

LCMS (ESI): 321.0 m/z [M+H]⁺

### Step I

To a solution of 6-[3-(tert-butoxy)-3-oxopropoxy]-2-methyl-1H-1,3-benzodiazole-4-carboxylic acid (41.6 mg, 0.130 mmol) and 3-aminopiperidine-2,6-dione hydrochloride (25.7 mg, 0.156 mmol) in DMF were added DIPEA (0.068 mL, 0.390 mmol) and HATU (98.9 mg, 0.260 mmol). The mixture was stirred at RT for 30 min. Then, the crude was purified by reverse phase flash chromatography (C18, H₂O:MeCN+0.1% FA) to obtain tert-butyl 3-({4-[(2,6-dioxopiperidin-3-yl)carbamoyl]-2-methyl-1H-1,3-benzodiazol-6-yl}oxy)propanoate (22.0 mg, 0.051 mmol, 39.3%) as a white solid.

LCMS (ESI): 431.3 m/z [M+H]⁺

### Step J

To a solution of tert-butyl 3-({4-[(2,6-dioxopiperidin-3-yl)carbamoyl]-2-methyl-1H-1,3-benzodiazol-6-yl}oxy)propanoate in DCM was added TFA and the mixture was stirred for next 18 h at RT. Then, the crude was concentrated in vacuo and dissolved in 1 M HCl in water. The solvent was evaporated under reduced pressure, redissolved in water and freeze-dried to obtain 3-({4-[(2,6-dioxopiperidin-3-yl)carbamoyl]-2-methyl-1H-1,3-benzodiazol-6-yl}oxy)propanoic acid hydrochloride (18.3 mg, 0.045 mmol, 87.2%) as an off-white solid.

LCMS (ESI): 375.1 m/z [M+H]⁺

### Step K

To a solution of 3-({4-[(2,6-dioxopiperidin-3-yl)carbamoyl]-2-methyl-1H-1,3-benzodiazol-6-yl}oxy)propanoic acid hydrochloride (18.3 mg, 0.044 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) and HATU (22.6 mg, 0.059 mmol) in dry DMF (2.0 mL) was added DIPEA (0.026 mL, 0.148 mmol). The mixture was stirred at RT for 30 min. The crude was diluted with DCM and washed with brine. The organic layer was dried over Na₂SO₄, filtered and concentrated in vacuo to get 33.5 mg crude of tert-butyl 6-chloro-1-(2-{4-[3-({4-[(2,6-dioxopiperidin-3-yl)carbamoyl]-2-methyl-1H-1,3-benzodiazol-6-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate as yellow oil which was used in next step without further purification.

LCMS (ESI): 1030.5 m/z [M+H]⁺

### Step L

To a solution of tert-butyl 6-chloro-1-(2-{4-[3-({4-[(2,6-dioxopiperidin-3-yl)carbamoyl]-2-methyl-1H-1,3-benzodiazol-6-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (33.5 mg, crude) in DCM (1.0 mL) was added TFA (1.0 mL, 13.059 mmol). The reaction mixture was stirred for 18 h at RT. The crude was concentrated in vacuo and purified by preparative HPLC (C18, H₂O:MeCN+0.1% FA) to obtain 6-chloro-1-(2-{4-[3-({4-[(2,6-dioxopiperidin-3-yl)carbamoyl]-2-methyl-1H-1,3-benzodiazol-6-yl}oxy)propanoyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (9.9 mg, 0.010 mmol, 33% over 2 steps) as white solid.

LCMS (ESI): 974.2 m/z [M+H]⁺

¹H NMR (500 MHz, DMSO, 353 K) δ 12.65 (s, 1H), 10.55 (s, 1H), 10.19 (s, 1H), 8.25 (dd, J=9.3, 5.9 Hz, 1H), 7.66 (d, J=8.4 Hz, 1H), 7.61-7.54 (m, 1H), 7.45-7.37 (m, 3H), 7.32 (td, J=8.9, 2.6 Hz, 1H), 7.24-7.15 (m, 2H), 6.87 (dd, J=6.2, 2.4 Hz, 1H), 4.87-4.78 (m, 1H), 4.40-4.15 (m, 6H), 3.75 (s, 3H), 3.41-3.35 (m, 4H), 3.30-3.24 (m, 2H), 2.85-2.70 (m, 4H), 2.66-2.56 (m, 1H), 2.54 (s, 3H), 2.29-2.20 (m, 2H), 2.19-2.06 (m, 7H), 2.01 (d, J=2.6 Hz, 3H), 1.90-1.86 (m, 3H).

### Example 3: 6-Chloro-1-(2-(4-(2-((4-((2,6-dioxopiperidin-3-yl)carbamoyl)-2-methyl-1H-benzo[d]imidazol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 2)

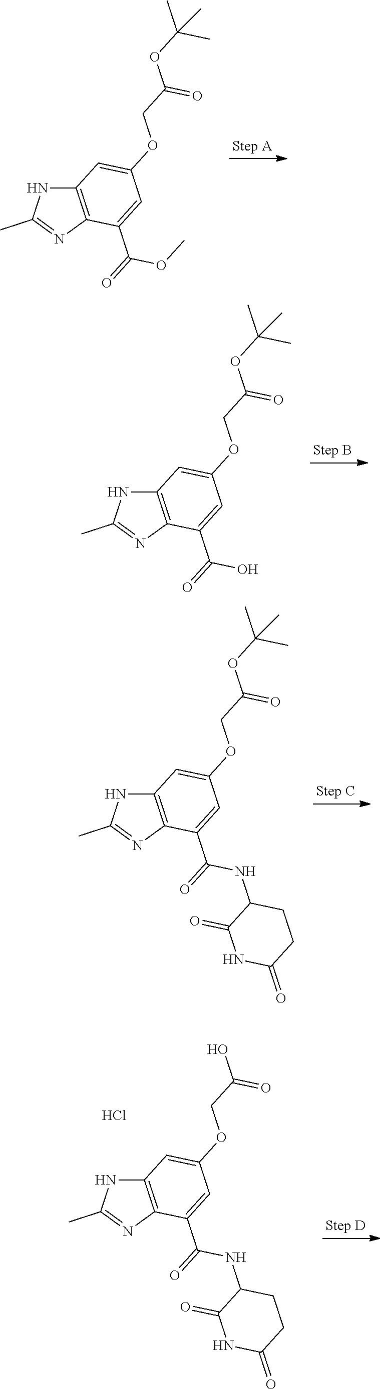

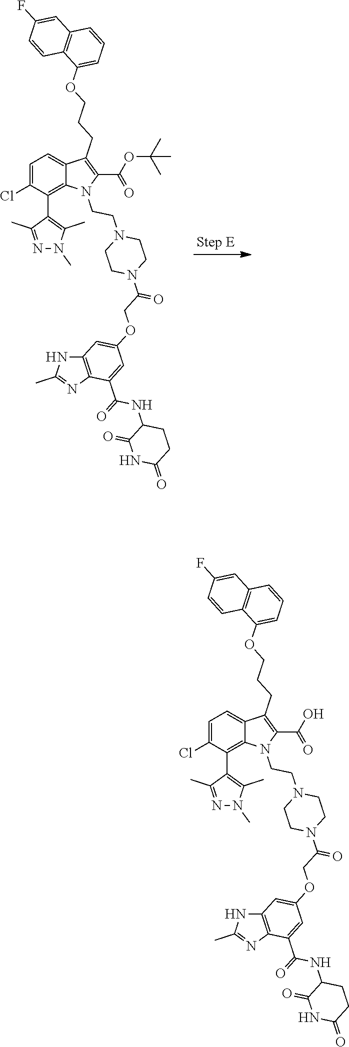 [SMILES: Cc1nc2c(C(=O)NC3CCC(=O)NC3=O)cc(OCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2[nH]1 ; Cc1nc2c(C(=O)NC3CCC(=O)NC3=O)cc(OCC(=O)N3CCN(CCn4c(C(=O)O)c(CCCOc5cccc6cc(F)ccc56)c5ccc(Cl)c(-c6c(C)nn(C)c6C)c54)CC3)cc2[nH]1]

### Step A

To a solution of methyl 6-[2-(tert-butoxy)-2-oxoethoxy]-2-methyl-1H-1,3-benzodiazole-4-carboxylate (50.0 mg, 0.156 mmol) in H₂O (0.500 mL) and MeCN (2.5 mL) was added LiBr (271.1 mg, 3.122 mmol) and Et₃N (0.218 mL, 1.561 mmol). The mixture was stirred for 6 days at room temperature. The crude was concentrated under reduced pressure and purified with reverse phase chromatography (C18, H₂O:MeCN+0.1% FA) to get 6-[2-(tert-butoxy)-2-oxoethoxy]-2-methyl-1H-1,3-benzodiazole-4-carboxylic acid (9.4 mg, 0.031 mmol, 19.7%) as a white solid.

LCMS (ESI): 307.1 m/z [M+H]⁺

### Step B

6-[2-(tert-Butoxy)-2-oxoethoxy]-2-methyl-1H-1,3-benzodiazole-4-carboxylic acid (9.4 mg, 0.031 mmol), 3-aminopiperidine-2,6-dione hydrochloride (6.1 mg, 0.037 mmol) and HATU (17.5 mg, 0.046 mmol) were dissolved in dry DMF (1.0 mL) under argon atmosphere and DIPEA (0.016 mL, 0.092 mmol) was added. Reaction (monitored with LCMS) was stirred for 15 min at room temperature under argon. After complete consumption of the starting material the solution was diluted with DCM up to 10 mL and washed with aqueous NaHCO₃ (10 mL), brine (10 mL) and water (10 mL). Organic layer was dried over anhydrous magnesium sulphate, filtered and dried under reduced pressure to give tert-butyl 2-({4-[(2,6-dioxopiperidin-3-yl)carbamoyl]-2-methyl-1H-1,3-benzodiazol-6-yl}oxy)acetate (10.1 mg, 0.24 mmol, 78.7%) as yellow oil. Reaction product was submitted to the next step without further purification.

LCMS (ESI): 417.4 m/z [M+H]⁺

### Step C

tert-Butyl 2-({4-[(2,6-dioxopiperidin-3-yl)carbamoyl]-2-methyl-1H-1,3-benzodiazol-6-yl}oxy)acetate (10.1 mg, 0.024 mmol) was dissolved in dry DCM (0.250 mL) under argon atmosphere and TFA (0.250 mL, 3.265 mmol) was added. Reaction was stirred under argon at room temperature for 16 h. Upon LCMS indicated full consumption of the starting material, the solution was concentrated under reduced pressure. 1M aqueous HCl (1 mL) was added and the solution concentrated to under vacuum. The addition of HCl followed by evaporation was repeated twice. Product was dry-freezed to give crude 2-({4-[(2,6-dioxopiperidin-3-yl)carbamoyl]-2-methyl-1H-1,3-benzodiazol-6-yl}oxy)acetic acid hydrochloride (8.0 mg, 0.020 mmol, 83.3%) as white solid.

LCMS (ESI): 361.0 m/z [M+H]⁺

### Step D

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (11.3 mg, 0.017 mmol) and {4-[(2,6-dioxopiperidin-3-yl)carbamoyl]-2-methyl-1H-1,3-benzodiazol-6-yl}oxy)acetic acid hydrochloride (8.0 mg, 0.020 mmol) were dissolved in dry DMF (0.8 mL) under argon atmosphere and DIPEA (0.009 mL, 0.050 mmol), followed by HATU (6.7 mg, 0.018 mmol) were added. Reaction (monitored with LCMS) was stirred under argon for 20 min at room temperature. After complete consumption of the starting material the mixture was diluted with DCM up to 10 mL and washed with saturated aqueous NaHCO₃ (10 mL), brine (10 mL) and water (10 mL). Organic layer was dried over anhydrous sodium sulphate, filtered and dried under reduced pressure to give crude tert-butyl 6-chloro-1-(2-{4-[2-({4-[(2,6-dioxopiperidin-3-yl)carbamoyl]-2-methyl-1H-1,3-benzodiazol-6-yl}oxy)acetyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (14.2 mg) as yellow oil, that was used in the subsequent step without further purification.

LCMS (ESI): 1016.1 m/z [M+H]⁺

### Step E

Crude tert-butyl 6-chloro-1-(2-{4-[2-({4-[(2,6-dioxopiperidin-3-yl)carbamoyl]-2-methyl-1H-1,3-benzodiazol-6-yl}oxy)acetyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (14.2 mg, 0.014 mmol) was dissolved under argon atmosphere in dry DCM (0.500 mL) and TFA (0.500 mL, 6.529 mmol) were added. Reaction (monitored with LCMS) was stirred for 16 h at room temperature under argon. After complete conversion of the starting material the solution was concentrated to dryness under reduced pressure and purified with reverse phase preparative HPLC (C18, H₂O:MeCN+0.1% FA) to give 6-chloro-1-(2-(4-(2-((4-((2,6-dioxopiperidin-3-yl)carbamoyl)-2-methyl-1H-benzo[d]imidazol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (7.3 mg, 0.008 mmol, 47% over 2 steps) as white solid.

LCMS (ESI): 960.1 m/z [M+H]⁺

¹H NMR (500 MHz, DMSO, 353 K) δ 12.34 (s, 1H), 10.58 (s, 1H), 10.20 (s, 1H), 8.26 (dd, J=9.2, 5.9 Hz, 1H), 7.72 (d, J=8.6 Hz, 1H), 7.60 (dd, J=10.4, 2.6 Hz, 1H), 7.51-7.38 (m, 3H), 7.34 (td, J=8.9, 2.6 Hz, 1H), 7.22 (d, J=8.5 Hz, 1H), 7.21-7.16 (m, 1H), 6.88 (dd, J=5.9, 2.8 Hz, 1H), 4.91-4.81 (m, 1H), 4.79 (s, 2H), 4.36-4.24 (m, 3H), 4.21 (ddd, J=14.0, 7.9, 5.8 Hz, 1H), 3.76 (s, 3H), 3.45-3.36 (m, 4H), 3.34-3.25 (m, 2H), 2.81 (ddd, J=17.5, 13.0, 5.6 Hz, 1H), 2.65-2.59 (m, 1H), 2.55 (s, 3H), 2.35-2.21 (m, 3H), 2.20-2.05 (m, 7H), 2.02 (s, 3H), 1.90 (s, 3H).

### Example 4: 6-Chloro-1-{2-[4-(2-{[3-(2,6-dioxopiperidin-3-yl)-2-methylquinolin-6-yl]formamido}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 5)

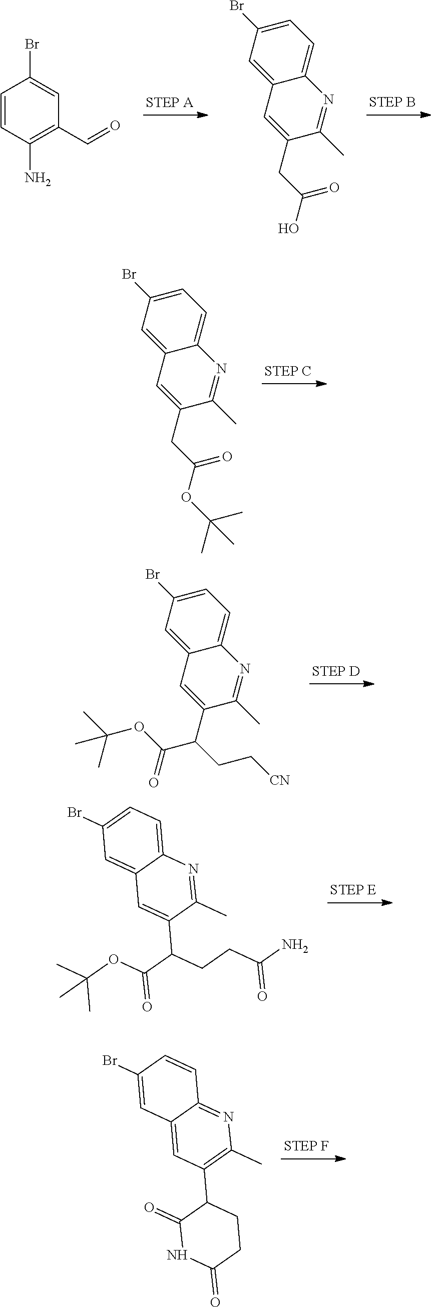

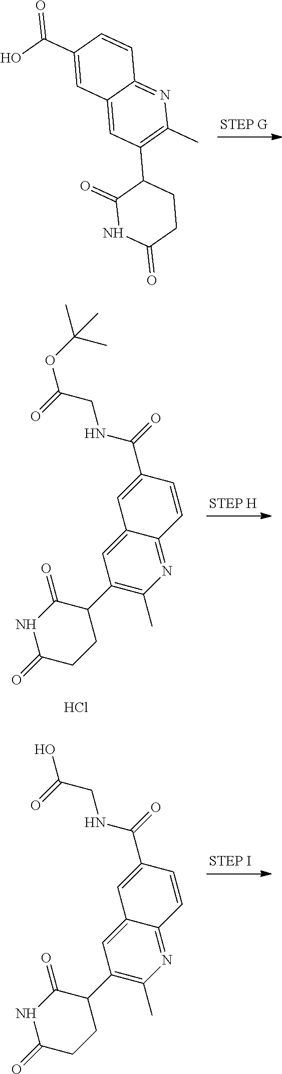 [SMILES: Cc1nc2ccc(C(=O)O)cc2cc1C1CCC(=O)NC1=O ; Cc1nc2ccc(C(=O)NCC(=O)OC(C)(C)C)cc2cc1C1CCC(=O)NC1=O ; Cl ; Cc1nc2ccc(C(=O)NCC(=O)O)cc2cc1C1CCC(=O)NC1=O]

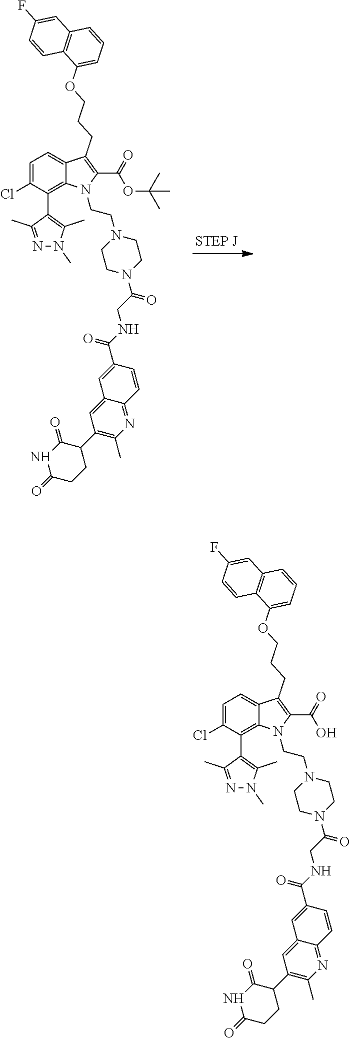 [SMILES: Cc1nc2ccc(C(=O)NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)cc2cc1C1CCC(=O)NC1=O ; Cc1nc2ccc(C(=O)NCC(=O)N3CCN(CCn4c(C(=O)O)c(CCCOc5cccc6cc(F)ccc56)c5ccc(Cl)c(-c6c(C)nn(C)c6C)c54)CC3)cc2cc1C1CCC(=O)NC1=O]

### Step A

To a stirred solution of 2-amino-5-bromobenzaldehyde (5.8 g, 28.99 mmol) in MeOH (140 mL) was added 4-oxopentanoic acid (10.34 mL, 101.48 mmol) followed by 2(N) aqueous sodium hydroxide solution (20 mL) dropwise at 0° C. Then, the resulting reaction mixture was allowed to reflux for 18 h. After complete consumption of the starting material, (monitored by TLC and LCMS), the reaction mixture was concentrated under reduced pressure. The residue was dissolved by minimum amount of water and washed with Et₂O. Then, the aqueous layer was neutralized with acetic acid and the obtained precipitate was filtered and washed with ether and pentane to get 2-(6-bromo-2-methylquinolin-3-yl)acetic acid (5 g, crude) as yellow solid which was directly used for next step without further purification.

LCMS (ESI): 280.0 m/z [M+H]⁺

### Step B

To a cooled solution of DCC (8.55 g, 41.43 mmol) in DCM (100 mL) was added DMAP (3.29 g, 26.93 mmol) at 0° C., followed by 2-(6-bromo-2-methylquinolin-3-yl)acetic acid (5 g, crude) and the resulting mixture was stirred at 0° C. for 5 min. Then, tert-butanol (3.071 mL, 103.57 mmol) was added and the reaction was allowed to stir at room temperature for 12 h under nitrogen. After complete consumption of the starting material, (monitored by TLC and LCMS), the solvent was evaporated under reduced pressure to get the crude compound, which was diluted with EtOAc and washed with water and brine. The organic layer was dried over Na₂SO₄ and evaporated under reduced pressure to get the crude compound, which was then purified with column chromatography (SiO₂, 30-40% EtOAc in hexane) to get tert-butyl 2-(6-bromo-2-methylquinolin-3-yl)acetate (4 g, 11.9 mmol, 41% over 2 steps) as off white solid.

LCMS (ESI): 335.4 m/z [M+H]⁺

### Step C

To a stirred solution of tert-butyl 2-(6-bromo-2-methylquinolin-3-yl)acetate (4 g, 11.9 mmol) in DMF (50 mL) were added K₂CO₃ (1.81 g, 13.095 mmol) followed by TEBAC (2.712 g, 11.905 mmol) under nitrogen atmosphere at 0° C. Then, acrylonitrile (0.858 mL, 13.095 mmol) was added and the resulting mixture was allowed to stir at room temperature for 3 h under nitrogen. After complete consumption of the starting material, (monitored by TLC and LCMS), the reaction mixture was diluted with EtOAc and washed successively with cold water and brine. The organic layer was dried over Na₂SO₄ and evaporated under reduced pressure to get the crude compound, which was then purified with column chromatography (SiO2, 30-50% EtOAc in hexane) to get tert-butyl 2-(6-bromo-2-methylquinolin-3-yl)-4-cyanobutanoate (1.4 g, 3.6 mmol, 30%) as light yellow sticky solid.

LCMS (ESI): 387.4 m/z [M−H]⁻

### Step D

To a solution of tert-butyl 2-(6-bromo-2-methylquinolin-3-yl)-4-cyanobutanoate (1.4 g, 3.6 mmol) in MeOH (18 mL) was added H₂O₂ (1.5 mL, 3.6 mmol), followed by K₂CO₃ (995 mg, 7.21 mmol) under nitrogen atmosphere at 0° C. Then the reaction mixture was allowed stir at room temperature for 16 h under nitrogen. After consumption of starting material (monitored by TLC and LCMS), the reaction mixture was concentrated under reduced pressure to get the crude compound which was then purified by column chromatography (SiO₂, 2-3% MeOH in DCM) to get tert-butyl 5-amino-2-(6-bromo-2-methylquinolin-3-yl)-5-oxopentanoate (700.0 mg, 1.72 mmol, 48%) as white solid.

LCMS (ESI): 409.1 m/z [M+H]⁺

### Step E

To stirred solution of tert-butyl 5-amino-2-(6-bromo-2-methylquinolin-3-yl)-5-oxopentanoate (700 mg, 1.72 mmol) in MeCN (10.0 mL) was added PTSA (980 g, 5.15 mmol) at room temperature and the reaction mixture was refluxed for 16 h. After complete consumption of the starting material (monitored by LC-MS and TLC), the reaction mixture was neutralized by adding Et₃N at 0° C. and then evaporated out to get crude compound which was then purified with column chromatography (SiO₂, 5% MeOH in DCM) to get 3-(6-bromo-2-methylquinolin-3-yl)piperidine-2,6-dione (350 mg, 1.05 mmol, 61%) as a white solid.

LCMS (ESI): 335.0 m/z [M+H]⁺

### Step F

A solution of 3-(6-bromo-2-methylquinolin-3-yl)piperidine-2,6-dione (350.0 mg, 1.05 mmol) in DMF (5.0 mL), H₂O (3 mL) and MeCN (5 mL), was added BINAP (65.34 mg, 0.105 mmol), Mo(CO)₆ (277.32 mg, 1.05 mmol) and CsF (160 mg, 1.05 mmol) at room temperature. The reaction mixture was de-oxygenated with argon for 10 minutes and then Pd(OAc)₂ (11.8 mg, 0.053 mmol) was added to it. The reaction mixture was allowed to stir at 90° C. for 16 hr. After complete consumption of the starting material (monitored by TLC and LCMS), the reaction mixture was filtered through a celite pad and the filtrate was purified with preparative HPLC (C18, H₂O:MeCN+0.1% FA) to get 3-(2,6-dioxopiperidin-3-yl)-2-methylquinoline-6-carboxylic acid (130 mg, 0.436 mmol, 41.5%) as white solid.

LCMS (ESI): 299.2 m/z [M+H]⁺

### Step G

To a solution of 3-(2,6-dioxopiperidin-3-yl)-2-methylquinoline-6-carboxylic acid (30.0 mg, 0.101 mmol) and tert-butyl 2-aminoacetate hydrochloride (25.3 mg, 0.151 mmol) in dry DMF (2.0 mL) were added HATU (76.5 mg, 0.201 mmol) and DIPEA (0.088 mL, 0.503 mmol). The reaction was stirred at room temperatyre for 15 min. The crude was purified with reverse phase flash chromatography (C18, H₂O:MeCN+0.1% FA) to obtain tert-butyl 2-{[3-(2,6-dioxopiperidin-3-yl)-2-methylquinolin-6-yl]formamido}acetate (39.0 mg, 0.095 mmol, 94%) as a white solid.

LCMS (ESI): 412.1 m/z [M+H]⁺

### Step H

To a solution of tert-butyl 2-{[3-(2,6-dioxopiperidin-3-yl)-2-methylquinolin-6-yl]formamido}acetate (39.0 mg, 0.095 mmol) in DCM (1.0 mL) was added TFA (1.0 mL). The mixture was stirred for 5 h at room temperature. The crude was concentrated under reduced pressure and dissolved in H₂O. To the solution was added HCl and it was evaporated. The product 2-{[3-(2,6-dioxopiperidin-3-yl)-2-methylquinolin-6-yl]formamido}acetic acid hydrochloride (37.0 mg, 0.094 mmol, 99.6%) was isolated as white solid.

LCMS (ESI): 356.0 m/z [M+H]⁺

### Step I

To a solution of 2-{[3-(2,6-dioxopiperidin-3-yl)-2-methylquinolin-6-yl]formamido}acetic acid hydrochloride (10.5 mg, 0.027 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) in dry DMF (2.0 mL) were added HATU (16.9 mg, 0.044 mmol) and DIPEA (0.019 mL, 0.111 mmol). The reaction was stirred at room temperature for 15 min. The crude was diluted in DCM and washed with brine. The organic layer was dried over MgSO₄, filtered and concentrated under reduced pressure to obtain tert-butyl 6-chloro-1-{2-[4-(2-{[3-(2,6-dioxopiperidin-3-yl)-2-methylquinolin-6-yl]formamido}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (43.8 mg, crude) as yellow oil, which was used in the next step without further purification.

LCMS (ESI): 1011.5 m/z [M+H]⁺

### Step J

To a solution of tert-butyl 6-chloro-1-{2-[4-(2-{[3-(2,6-dioxopiperidin-3-yl)-2-methylquinolin-6-yl]formamido}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (43.8 mg, crude) in DCM (1.0 mL) was added TFA (1.0 mL, 13.059 mmol). The reaction mixture was stirred for 18 h at room temperature. The crude was concentrated in vacuo and purified with preparative HPLC (C18, H₂O:MeCN+0.1% FA) to obtain 6-chloro-1-{2-[4-(2-{[3-(2,6-dioxopiperidin-3-yl)-2-methylquinolin-6-yl]formamido}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (2.2 mg, 0.002 mmol, 9% over 2 steps) as solid.

LCMS (ESI): 955.2 m/z [M+H]⁺

¹H NMR (500 MHz, DMSO, 353 K) δ 10.61 (s, 1H), 8.41 (d, J=2.0 Hz, 1H), 8.36 (t, J=5.6 Hz, 1H), 8.25 (dd, J=9.2, 5.9 Hz, 1H), 8.18 (s, 1H), 8.10 (dd, J=8.8, 2.0 Hz, 1H), 7.96 (d, J=8.8 Hz, 1H), 7.66 (d, J=8.5 Hz, 1H), 7.58 (dd, J=10.4, 2.6 Hz, 1H), 7.45-7.39 (m, 2H), 7.32 (td, J=8.9, 2.7 Hz, 1H), 7.18 (d, J=8.4 Hz, 1H), 6.87 (dd, J=5.8, 2.8 Hz, 1H), 4.35-4.27 (m, 2H), 4.24 (t, J=6.4 Hz, 2H), 4.23-4.16 (m, 1H), 4.14 (d, J=5.5 Hz, 2H), 3.77 (s, 3H), 3.42-3.36 (m, 4H), 3.30-3.23 (m, 2H), 2.83 (ddd, J=17.6, 12.5, 5.5 Hz, 1H), 2.70 (s, 3H), 2.69-2.61 (m, 1H), 2.48-2.36 (m, 1H), 2.29-2.07 (m, 9H), 2.02 (s, 3H), 1.89 (s, 3H).

### Example 5: 6-Chloro-1-(2-(4-((3-(2,6-dioxopiperidin-3-yl)-2-methylquinoline-7-carbonyl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 6)

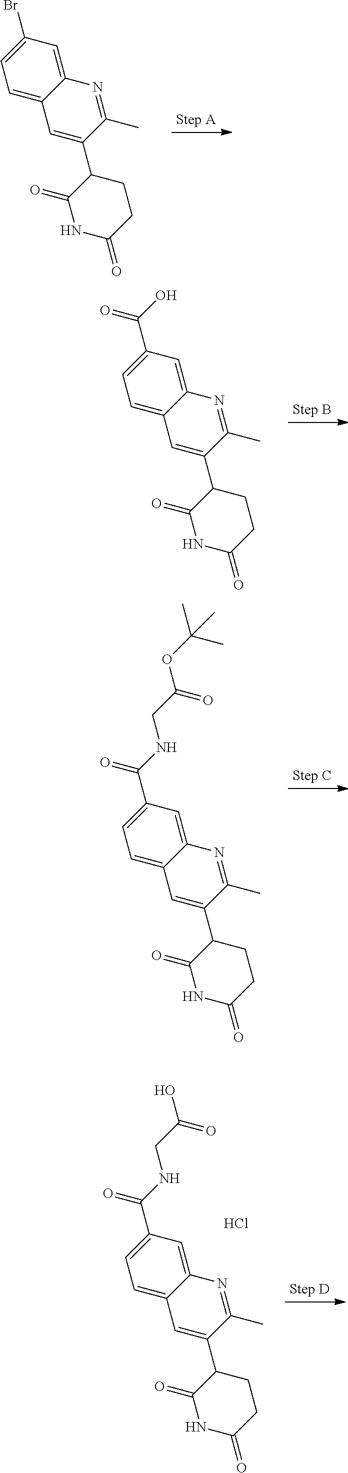

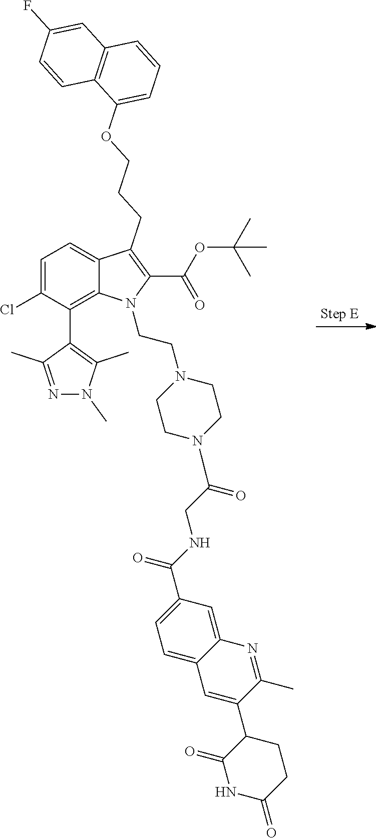

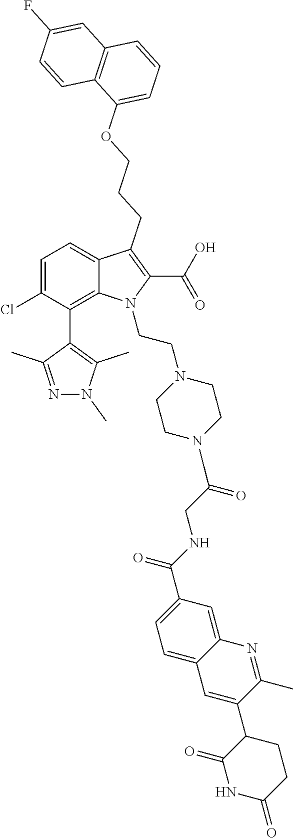

### Step A

A solution of 3-(7-bromo-2-methylquinolin-3-yl)piperidine-2,6-dione (1.0 g, 3.012 mmol), Mo(CO)₆ (0.795 g, 3.012 mmol) in MeCN (10 mL), DMF (10 mL) and water (8.0 mL) was added cesium fluoride (0.457 g, 3.012 mmol). The reaction mixture was then deoxygenated with argon for 10 min. BINAP (0.187 g, 0.301 mmol) followed by Pd(OAc)₂ (0.034 g, 0.151 mmol) were added to the reaction mixture and it was allowed to stir at 90° C. for 16 h. After complete consumption of the starting material (monitored by TLC and LCMS) the mixture was filtered through celite and washed with MeCN. The filtrate was concentrated under reduced pressure and crude material was purified with reverse phase preparative HPLC (C18, 10 mM ammonium acetate in H₂O:MeCN) to afford 3-(2,6-dioxopiperidin-3-yl)-2-methylquinoline-7-carboxylic acid (325 mg, 1.1 mmol, 36.5%) as off white solid.

LCMS (ESI): 299.0 m/z [M+H]⁺

### Step B

3-(2,6-Dioxopiperidin-3-yl)-2-methylquinoline-7-carboxylic acid (30.0 mg, 0.101 mmol), tert-butyl 2-aminoacetate hydrochloride (25.3 mg, 0.151 mmol) and HATU (76.5 mg, 0.201 mmol) were dissolved in dry DMF (2.0 mL) under argon atmosphere and DIPEA (0.088 mL, 0.503 mmol) was added. Reaction (monitored with LCMS) was stirred for 30 min. After complete consumption of the starting material the solution was diluted with DCM up to 10 mL and washed with brine (2×10 mL) and water (2×10 mL). The organic layer was dried over anhydrous magnesium sulphate, filtered and concentrated under reduced pressure. The resulting residue was dissolved in DMSO and purified with preparative HPLC (C18, H₂O:MeCN+0.1% FA) to give tert-butyl 2-{[3-(2,6-dioxopiperidin-3-yl)-2-methylquinolin-7-yl]formamido}acetate (34.9 mg, 0.085 mmol, 84.4%) as white solid.

LCMS (ESI): 412.1 m/z [M+H]⁺

### Step C

tert-Butyl 2-{[3-(2,6-dioxopiperidin-3-yl)-2-methylquinolin-7-yl]formamido}acetate (34.9 mg, 0.085 mmol) was dissolved in dry DCM (0.5 mL) under argon atmosphere and TFA (0.500 mL, 6.529 mmol) was added. Reaction (monitored with LCMS) was stirred at room temperature under argon for 16 h. After complete consumption of the starting material the solution was concentrated under reduced pressure. The resulting residue was dissolved in 1M HCl (1.0 mL) and the solution was concentrated to dryness. The addition and evaporation of aqueous HCl was twice repeated and the resulting product was dry-freezed to give 2-{[3-(2,6-dioxopiperidin-3-yl)-2-methylquinolin-7-yl]formamido}acetic acid hydrochloride (29.5 mg, 0.075 mmol, 88.7%) as white solid.

LCMS (ESI): 356.0 m/z [M+H]⁺

### Step D

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (23.5 mg, 0.035 mmol) and 2-{[3-(2,6-dioxopiperidin-3-yl)-2-methylquinolin-7-yl]formamido}acetic acid hydrochloride (15.0 mg, 0.038 mmol) were dissolved in dry DMF (1.0 mL) and DIPEA (0.018 mL, 0.105 mmol) was added. To it, HATU (13.9 mg, 0.037 mmol) was added as a solution in DMF (1.0 mL) and the reaction (monitored with LCMS) was stirred at room temperature for 40 min. After complete consumption of the starting material the solution was diluted with DCM up to 10 mL and washed with brine (2×10 mL) and water (2×10 mL). Organic layer was dried over anhydrous magnesium sulphate, filtered and concentrated under reduced pressure. Crude tert-butyl 6-chloro-1-{2-[4-(2-{[3-(2,6-dioxopiperidin-3-yl)-2-methyl quinolin-7-yl]formamido}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (44.7 mg) obtained as yellow oil was used in the next step without further purification.

LCMS (ESI): 1011.47 m/z [M+H]⁺

### Step E

Crude tert-butyl 6-chloro-1-{2-[4-(2-{[3-(2,6-dioxopiperidin-3-yl)-2-methylquinolin-7-yl]formamido}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (44.7 mg, 0.044 mmol) was dissolved under argon atmosphere in dry DCM (0.5 mL). To it, TFA (0.500 mL, 6.529 mmol) was added and the reaction (monitored with LCMS) was stirred for 16 h at room temperature under argon. After complete consumption of the starting material the mixture was concentrated under reduced pressure. The resulting residue was dissolved in DMSO and purified with reverse phase preparative HPLC (C18, H₂O:MeCN+0.1% FA) to give 6-chloro-1-(2-(4-((3-(2,6-dioxopiperidin-3-yl)-2-methyl quinoline-7-carbonyl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (14.4 mg, 0.015 mmol, 42.8% over 2 steps) as white solid.

LCMS (ESI): 955.2 m/z [M+H]⁺

¹H NMR (500 MHz, DMSO, 353 K) δ 10.62 (s, 1H), 8.54-8.40 (m, 2H), 8.24 (dd, J=9.2, 5.8 Hz, 1H), 8.14 (s, 1H), 7.94 (s, 2H), 7.71 (d, J=8.6 Hz, 1H), 7.59 (dd, J=10.4, 2.8 Hz, 1H), 7.47-7.38 (m, 2H), 7.33 (td, J=8.9, 2.7 Hz, 1H), 7.21 (d, J=8.4 Hz, 1H), 6.88 (dd, J=5.5, 3.0 Hz, 1H), 4.38-4.08 (m, 7H), 3.78 (s, 3H), 3.43-3.38 (m, 4H), 3.29 (t, J=7.4 Hz, 2H), 2.83 (ddd, J=17.4, 12.5, 5.3 Hz, 1H), 2.71 (s, 3H), 2.69-2.62 (m, 1H), 2.47-2.35 (m, 1H), 2.30-2.06 (m, 9H), 2.02 (s, 3H), 1.89 (s, 3H).

### Example 6: 6-Chloro-1-{2-[4-(2-{[3-(2,6-dioxopiperidin-3-yl)-2-methylquinolin-8-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid, formate salt (Compound 4)

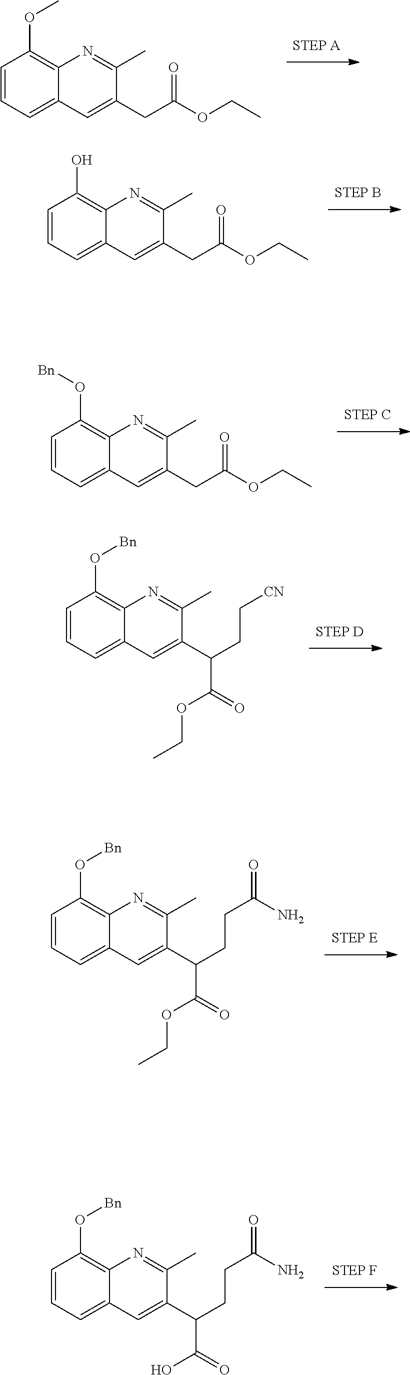

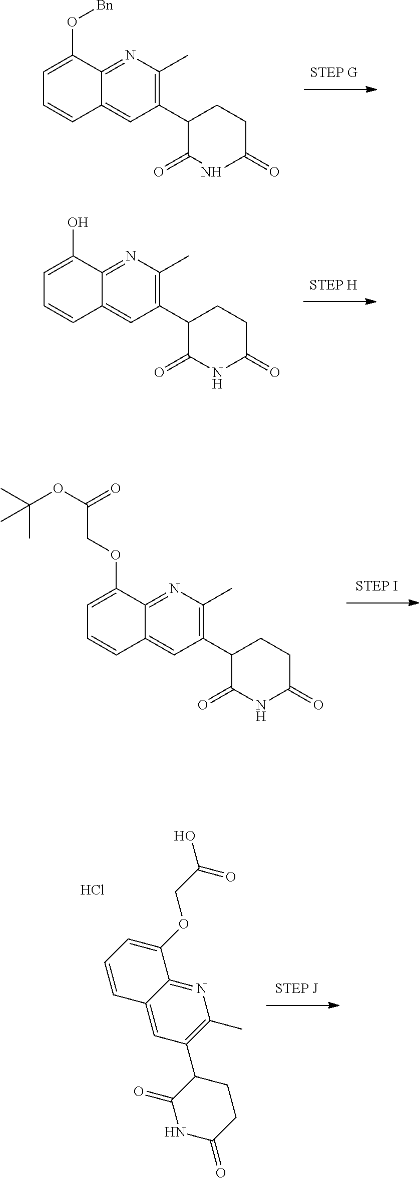 [SMILES: Cc1nc2c(OCc3ccccc3)cccc2cc1C1CCC(=O)NC1=O ; Cc1nc2c(O)cccc2cc1C1CCC(=O)NC1=O ; Cc1nc2c(OCC(=O)OC(C)(C)C)cccc2cc1C1CCC(=O)NC1=O ; Cl ; Cc1nc2c(OCC(=O)O)cccc2cc1C1CCC(=O)NC1=O]

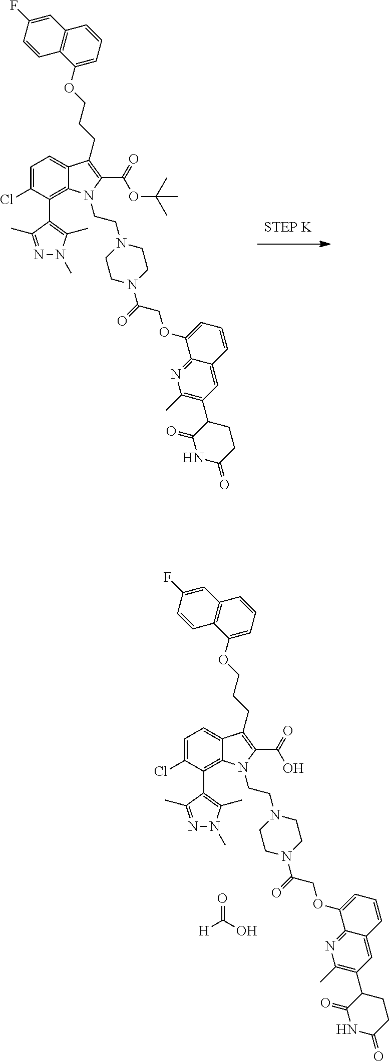 [SMILES: Cc1nc2c(OCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)cccc2cc1C1CCC(=O)NC1=O ; Cc1nc2c(OCC(=O)N3CCN(CCn4c(C(=O)O)c(CCCOc5cccc6cc(F)ccc56)c5ccc(Cl)c(-c6c(C)nn(C)c6C)c54)CC3)cccc2cc1C1CCC(=O)NC1=O ; O=CO]

### Step A

To a stirred solution of ethyl 2-(8-methoxy-2-methylquinolin-3-yl)acetate (4.2 g, 16.2 mmol) in DCM (70 mL) at 0° C. was added 1 (M) solution of BBr₃ in DCM (65 mL, 64.8 mmol). The mixture was stirred at the same temperature for 4 h. After complete consumption of the starting material, (monitored by TLC and LCMS), the reaction mixture was poured into water at 0° C. and extracted with DCM. Then aqueous part was diluted with saturated NaHCO₃ solution and then extracted with DCM. The combined organic layer was washed with brine and then dried over Na₂SO₄, filtered and concentrated. Cude product was purified with column chromatography (SiO₂, 30% EtOAc in hexane) to get ethyl 2-(8-hydroxy-2-methylquinolin-3-yl)acetate (2.3 g, 9.38 mmol, 58%) as white solid.

LCMS (ESI): 245.8 m/z [M+H]⁺

### Step B

To a suspension of ethyl 2-(8-hydroxy-2-methylquinolin-3-yl)acetate (2.3 g, 9.38 mmol) in MeCN (40 mL) was added K₂CO₃ (2.5 g, 18.77 mmol), followed by benzyl bromide (0.7 mL, 12.20 mmol) at 0° C. The reaction mixture was allowed to stir at room temperature for 18 h under nitrogen. After complete consumption of the starting material, (monitored by TLC and LCMS), the volatiles were evaporated under reduced pressure. The resulting residue was diluted with EtOAc and washed with cold water and brine. The organic layer was dried over Na₂SO₄ and evaporated under reduced pressure to get the crude compound which was then purified with column chromatography (SiO₂, 40% EtOAc in hexane) to get ethyl 2-(8-(benzyloxy)-2-methylquinolin-3-yl)acetate (1.75 g, 5.22 mmol, 56%) as brown solid.

LCMS (ESI): 335.8 m/z [M+H]⁺

### Step C

To a solution of ethyl 2-(8-(benzyloxy)-2-methylquinolin-3-yl)acetate (1.75 g, 5.22 mmol) in DMF (25 mL) was added K₂CO₃ (1.4 g, 10.44 mmol), followed by TEBAC (1.2 g, 5.22 mmol) under nitrogen atmosphere at 0° C. Then acrylonitrile (0.45 mL, 6.791 mmol) was added and the mixture was allowed to stir at room temperature for 2 h under nitrogen. After complete consumption of the starting material, (monitored by TLC and LCMS), the reaction mixture was diluted with EtOAc and washed with cold water and brine. The organic layer was dried over anhydrous Na₂SO₄ and evaporated under reduced pressure to get the crude compound, which was then purified with column chromatography (SiO₂, 40% EtOAc in hexane) to get ethyl 2-(8-(benzyloxy)-2-methylquinolin-3-yl)-4-cyanobutanoate (800 mg, 2.06 mmol, 39%) as brown sticky Liquid.

LCMS (ESI): 388.8 m/z [M+H]⁺

### Step D

To a solution of ethyl 2-(8-(benzyloxy)-2-methylquinolin-3-yl)-4-cyanobutanoate (800 mg, 2.06 mmol) in MeOH (10 mL) was added H₂O₂ (0.3 mL, 10.31 mmol), followed by K₂CO₃ (56 mg, 0.412 mmol) under nitrogen atmosphere at 0° C. The mixture was allowed stir at room temperature for 16 h under nitrogen. After complete consumption of the starting material, (monitored by TLC and LCMS), the volatiles were removed under reduced pressure to get crude ethyl 5-amino-2-(8-(benzyloxy)-2-methylquinolin-3-yl)-5-oxopentanoate, which was directly used in next step without further purification.

LCMS (ESI): 407.3 m/z [M+H]⁺

### Step E

Ethyl 5-amino-2-(8-(benzyloxy)-2-methylquinolin-3-yl)-5-oxopentanoate (crude product from Step D) was dissolved in EtOH (10 mL) and a solution of LiOH (177 mg, 7.389 mmol) in water (2 mL) was added to it. The reaction mixture was allowed to stir for 16 h. After complete consumption of the starting material (monitored by TLC and LCMS), volatiles were evaporated under reduced pressure. Crude 5-amino-2-(8-(benzyloxy)-2-methylquinolin-3-yl)-5-oxopentanoic acid was used in the next step without further purification.

LCMS (ESI): 379.0 m/z [M+H]⁺

### Step F

Crude 5-amino-2-(8-(benzyloxy)-2-methylquinolin-3-yl)-5-oxopentanoic acid from previous step was dissolved in MeCN (20 mL) and p-toluenesulfonic acid monohydrate (3.4 g, 19.84 mmol) was added and the resulting reaction mixture was allowed to stir at 80° C. for 16 h. After the reaction, (monitored by TLC and LCMS), was completed, the mixture was concentrated under reduced pressure. Crude compound was purified with column chromatography (SiO₂, 70% EtOAc in DCM) to get 3-(8-(benzyloxy)-2-methylquinolin-3-yl)piperidine-2,6-dione (280 mg, 0.78 mmol, 38% over three steps) as brown solid.

LCMS (ESI): 361.1 m/z [M+H]⁺

### Step G

3-(8-(benzyloxy)-2-methylquinolin-3-yl)piperidine-2,6-dione (280 mg, 0.78 mmol) was dissolved in MeCN (10 mL) and 140 mg of Pd(OH)₂ (50% wt) was added. The reaction vessel was backfilled with hydrogen and the reaction mixture was allowed to stir for 8 h under hydrogen atmosphere. After complete consumption of the starting material, (monitored with TLC and LCMS), the reaction mixture was filtered through celite pad and washed thoroughly with MeCN. The combined filtrate was evaporated under reduced pressure to get the crude compound, which was then purified by triturating with ether and pentane to get 3-(8-hydroxy-2-methylquinolin-3-yl)piperidine-2,6-dione (115 mg, 0.43 mmol, 55%) as white solid.

LCMS (ESI): 271.1 m/z [M+H]⁺

### Step H

3-(8-hydroxy-2-methylquinolin-3-yl)piperidine-2,6-dione (25.0 mg, 0.092 mmol) was dissolved in DMF (2.0 mL) and KI (15.4 mg, 0.092 mmol), KHCO₃ (27.8 mg, 0.277 mmol) and tert-butyl bromoacetate (0.016 mL, 0.111 mmol) were added. Reaction was stirred at 60° C. for 6 h. The crude was injected directly to reverse phase flash chromatography (C18, H₂O:MeCN+0.1% FA) to give a corresponding tert-butyl 2-{[3-(2,6-dioxopiperidin-3-yl)-2-methylquinolin-8-yl]oxy}acetate (14.6 mg, 0.038 mmol, 41%) as a white solid.

LCMS (ESI): 385.2 m/z [M+H]⁺

### Step I

To a solution of tert-butyl 2-{[3-(2,6-dioxopiperidin-3-yl)-2-methylquinolin-8-yl]oxy}acetate (14.6 mg, 0.038 mmol) in DCM (1.0 mL) was added TFA (1.0 mL, 13.059 mmol). The mixture was stirred for 5 h at room temperature. The crude was concentrated in vacuo and dissolved in H₂O. To the solution was added HCl and it was evaporated. 2-{[3-(2,6-dioxopiperidin-3-yl)-2-methylquinolin-8-yl]oxy}acetic acid hydrochloride (12.6 mg, 0.035 mmol, 91.0%) was isolated as greenish solid.

LCMS (ESI): 329.0 m/z [M+H]⁺

### Step J

To a solution of 2-{[3-(2,6-dioxopiperidin-3-yl)-2-methylquinolin-8-yl]oxy}acetic acid hydrochloride (9.7 mg, 0.027 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) in dry DMF (2.0 mL) were added HATU (16.9 mg, 0.044 mmol) and DIPEA (0.012 mL, 0.067 mmol). The reaction was stirred at room temperature for 15 min. The crude was diluted in DCM and washed with brine. The organic layer was dried over MgSO₄, filtered and concentrated in vacuo to obtain tert-butyl 6-chloro-1-(2-{4-[(2-{[3-(2,6-dioxopiperidin-3-yl)-2-methylquinolin-8-yl]oxy}acetyl)oxy]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylate (37.5 mg, crude) as yellow oil, which was used in next step without further purification.

LCMS (ESI): 984.5 m/z [M+H]⁺

### Step K

To a solution of tert-butyl 6-chloro-1-{2-[4-(2-{[3-(2,6-dioxopiperidin-3-yl)-2-methylquinolin-8-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (37.5 mg, crude) in DCM (1.0 mL) was added TFA (1.0 mL, 13.059 mmol). The reaction mixture was stirred for 18 h at room temperature. The crude was concentrated in vacuo and purified with preparative HPLC (C18, H₂O:MeCN+0.1% FA) to obtain 6-chloro-1-{2-[4-(2-{[3-(2,6-dioxopiperidin-3-yl)-2-methylquinolin-8-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (formate salt) (10.3 mg, 0.011 mmol, 50% over 2 steps) as yellow solid.

LCMS (ESI): 928.2 m/z [M+H]⁺

¹H NMR (500 MHz, DMSO, 353 K) δ 10.59 (s, 1H), 8.24 (dd, J=9.2, 5.9 Hz, 1H), 8.02 (s, 1H), 7.68 (d, J=8.5 Hz, 1H), 7.57 (dd, J=10.4, 2.6 Hz, 1H), 7.44 (dd, J=8.2, 1.3 Hz, 1H), 7.42-7.36 (m, 3H), 7.32 (ddd, J=9.3, 8.7, 2.6 Hz, 1H), 7.19 (d, J=8.5 Hz, 1H), 7.09 (dd, J=7.7, 1.3 Hz, 1H), 6.86 (dd, J=5.4, 3.2 Hz, 1H), 4.98-4.93 (m, 2H), 4.33-4.21 (m, 4H), 4.18 (ddd, J=13.8, 7.9, 5.8 Hz, 1H), 3.72 (d, J=3.3 Hz, 3H), 3.52-3.39 (m, 4H), 3.30-3.23 (m, 2H), 2.81 (ddd, J=17.6, 12.5, 5.4 Hz, 1H), 2.67-2.61 (m, 4H), 2.43-2.31 (m, 1H), 2.28-2.19 (m, 2H), 2.19-2.04 (m, 7H), 2.00 (d, J=2.0 Hz, 3H), 1.88 (s, 3H).

### Example 7: 6-Chloro-1-{2-[4-(2-{[2-(2,6-dioxopiperidin-3-yl)-5-methoxy-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 7)

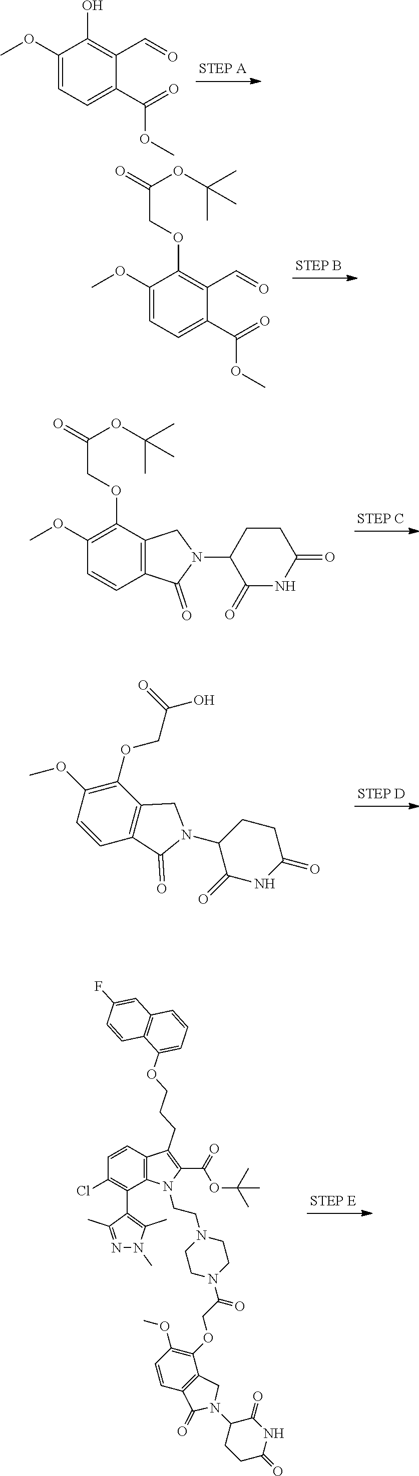

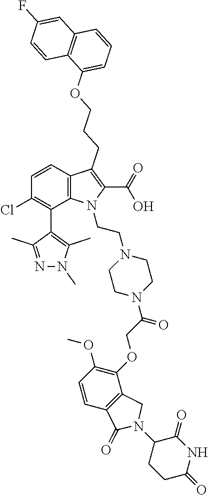 [SMILES: COc1ccc2c(c1OCC(=O)N1CCN(CCn3c(C(=O)O)c(CCCOc4cccc5cc(F)ccc45)c4ccc(Cl)c(-c5c(C)nn(C)c5C)c43)CC1)CN(C1CCC(=O)NC1=O)C2=O]

### Step A

To a solution of methyl 2-formyl-3-hydroxy-4-methoxybenzoate (550 mg, 2.62 mmol) in DMF (15 mL) was added K₂CO₃ (398 mg, 2.88 mmol) under nitrogen atmosphere at 0° C. Then, tert-butyl 2-bromoacetate (768 mg, 3.93 mmol) was added into the reaction mixture at room temperature. Then, the resulting mixture was allowed to stir at room temperature for 16 h under nitrogen. After complete consumption of the starting material, (monitored by TLC and LCMS), the reaction mixture was diluted in EtOAc and washed with cold water and brine. The organic layer was dried over anhydrous Na₂SO₄ and evaporated under reduced pressure to get the crude compound, which was then purified by column chromatography (SiO₂, 30% EtOAc in hexane) to get methyl 3-(2-(tert-butoxy)-2-oxoethoxy)-2-formyl-4-methoxybenzoate (600 mg, 1.85 mmol, 70%) as white sticky liquid.

LCMS (ESI): 325.0 m/z [M+H]⁺

### Step B

To a solution of methyl 3-(2-(tert-butoxy)-2-oxoethoxy)-2-formyl-4-methoxybenzoate (600 mg, 1.85 mmol) and 3-aminopiperidine-2,6-dione hydrochloride (610 mg, 3.7 mmol) in DCE (10 mL) was added acetic acid (0.1 mL, 1.85 mmol). The pH of the reaction mixture was adjusted to 7 and the reaction mixture was allowed to stir at reflux temperature for 4 h in sealed tube. Sodium triacetoxyborohydride (1.9 g, 9.26 mmol) was then added and the mixture was allowed to stir at 90° C. for 16 h. After complete consumption of the starting material, (monitored by TLC and LCMS), the volatiles were evaporated under reduced pressure to afford crude compound, which was purified by column chromatography (SiO₂, 80% EtOAc in DCM) to get tert-butyl 2-((2-(2,6-dioxopiperidin-3-yl)-5-methoxy-1-oxoisoindolin-4-yl)oxy)acetate (310 mg, 0.77 mmol, 42%) as brown solid.

LCMS (ESI): 405.0 m/z [M+H]⁺

### Step C

To a stirred suspension of tert-butyl 2-((2-(2,6-dioxopiperidin-3-yl)-5-methoxy-1-oxoisoindolin-4-yl)oxy)acetate (310 mg, 0.77 mmol) in DCM (5 mL) was added TFA (5 mL) dropwise at 0° C. under nitrogen. The mixture was allowed to stir at room temperature for 16 h. After complete consumption of the starting material, (monitored by TLC and LCMS), the volatiles were evaporated under reduced pressure to get the crude compound, which was then purified by triturating with ether and pentane to get 2-((2-(2,6-dioxopiperidin-3-yl)-5-methoxy-1-oxoisoindolin-4-yl)oxy)acetic acid (220 mg, 0.63 mmol, 81%) as black solid.

LCMS (ESI): 349.2 m/z [M+H]⁺

### Step D

To a mixture of 2-{[2-(2,6-dioxopiperidin-3-yl)-5-methoxy-1-oxo-2,3-dihydro-1H-isoindol-4-yl]oxy}acetic acid (9.3 mg, 0.027 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) and HATU (16.9 mg, 0.044 mmol) in dry DMF (2.0 mL) was added DIPEA (0.012 mL, 0.067 mmol) and the reaction was stirred for 30 min at room temperature. After complete consumption of a stating material, the crude was diluted with DCM and washed with brine. The organic layer was dried over MgSO₄, filtered and concentrated in vacuo to obtain tert-butyl 6-chloro-1-{2-[4-(2-{[2-(2,6-dioxopiperidin-3-yl)-5-methoxy-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (33.5 mg crude), which was used for next step without further purification.

LCMS (ESI): 1004.3 m/z [M+H]⁺

### Step E

To a solution of tert-butyl 6-chloro-1-{2-[4-(2-{[2-(2,6-dioxopiperidin-3-yl)-5-methoxy-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (33.5 mg, crude) in DCM (1.0 mL) was added TFA (1.0 mL, 13.059 mmol, 391.588 eq). The reaction mixture was stirred for 18 h at room temperature. The crude was concentrated in vacuo and purified by preparative HPLC (C18, H₂O:MeCN+0.1% FA) to obtain 6-chloro-1-{2-[4-(2-{[2-(2,6-dioxopiperidin-3-yl)-5-methoxy-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (13.5 mg, 0.014 mmol, 64% over two steps) as white solid.

LCMS (ESI): 948.2 m/z [M+H]⁺

¹H NMR (500 MHz, DMSO, 353 K) δ 10.62 (s, 1H), 8.24 (dd, J=9.2, 5.9 Hz, 1H), 7.70 (d, J=8.5 Hz, 1H), 7.58 (dd, J=10.3, 2.7 Hz, 1H), 7.44-7.39 (m, 3H), 7.32 (td, J=8.9, 2.7 Hz, 1H), 7.23-7.16 (m, 2H), 6.87 (dd, J=5.7, 2.9 Hz, 1H), 5.00 (dd, J=13.0, 5.2 Hz, 1H), 4.83-4.76 (m, 2H), 4.46 (dd, J=16.8, 2.3 Hz, 1H), 4.38 (dd, J=16.8, 2.9 Hz, 1H), 4.31-4.23 (m, 3H), 4.22-4.15 (m, 1H), 3.89 (s, 3H), 3.75 (s, 3H), 3.37-3.32 (m, 4H), 3.31-3.24 (m, 2H), 2.87 (ddd, J=17.1, 13.5, 5.5 Hz, 1H), 2.66-2.59 (m, 1H), 2.46-2.35 (m, 1H), 2.27-2.21 (m, 2H), 2.16-2.00 (m, 101H), 1.88 (s, 3H).

### Example 8: 6-Chloro-1-(2-(4-(2-((5-(dimethylamino)-2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 10)

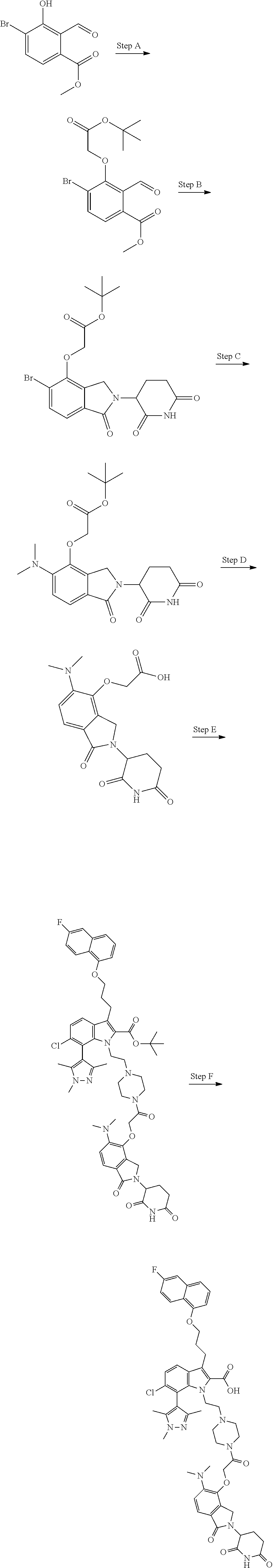

### Step A

To a solution of methyl 4-bromo-2-formyl-3-hydroxybenzoate (1.2 g, 4.631 mmol) in DMF (20 mL) was added potassium bicarbonate (1.926 g, 13.953 mmol), potassium iodide (772 mg, 4.651 mmol) and tert-butyl bromo acetate (0.684 ml, 4.651 mmol) at room temperature under nitrogen. The reaction mixture was allowed to stir at 60° C. for 1 h. After complete consumption of the starting material (monitored by TLC and LCMS) the reaction mixture was diluted with ethyl acetate and washed with water and brine. The organic layer was dried over sodium sulphate and evaporated under reduced pressure to get the crude compound, which was then purified by column chromatography (SiO₂, 20-30% EtOAc in DCM) to get methyl 4-bromo-3-(2-(tert-butoxy)-2-oxoethoxy)-2-formylbenzoate (1.6 g, 4.28 mmol, 92%) as off white solid.

LCMS (ESI): 373.0 m/z [M+H]⁺

### Step B

To a stirred solution of methyl 4-bromo-3-(2-(tert-butoxy)-2-oxoethoxy)-2-formylbenzoate (500 mg, 1.344 mmol) in DCE (10 mL) were added 3-aminopiperidine-2,6-dione hydrochloride (443.54 mg, 2.688 mmol) and acetic acid (0.077 ml, 1.344 mmol) at room temperature. The mixture was allowed to stir at 80° C. for 4 h. Then, the reaction mixture was cooled down to room temperature and sodium triacetoxyborohydride (0.7 g, 11.11 mol) was added. The resulting mixture was allowed then to stir at 80° C. for 16 h. After complete consumption of the starting material (monitored by TLC and LCMS) the volatiles were evaporated under reduced pressure. Crude compound was purified by column chromatography (SiO₂, 70% to 100% EtOAc in DCM) to get tert-butyl 2-((5-bromo-2-(2,6-dioxopiperidin-3-yl)-1-oxoisoindolin-4-yl)oxy)acetate (200 mg, 0.44 mmol, 33%) as off white solid.

LCMS (ESI): 453.0 m/z [M+H]⁺

### Step C

To a stirred solution of tert-butyl 2-((5-bromo-2-(2,6-dioxopiperidin-3-yl)-1-oxoisoindolin-4-yl)oxy)acetate (200 mg, 0.442 mmol) in dioxane (10 mL) were added dimethyl ammine in THF (2 M) (3.3 mL, 0.6.618 mmol) and cesium carbonate (121.546 mg, 0.126 mmol). The mixture was deoxygenated with argon and to it was added Pd-PEPPSI-IHeptCl (121.546 mg, 0.126 mmol) under argon atmosphere. The reaction mixture was then irradiated under microwave at 120° C. for 2 h. After complete consumption of the starting material (monitored by TLC and LCMS) the reaction mixture was filtered through celite pad and the solvents were evaporated under reduced pressure. Crude material purified with preparative TLC (SiO₂, 100% EtOAc) to afford tert-butyl 2-((5-(dimethylamino)-2-(2,6-dioxopiperidin-3-yl)-1-oxoisoindolin-4-yl)oxy)acetate (40 mg, 0.096 mmol, 21.7%) as yellow solid.

LCMS (ESI): 418.3 m/z [M+H]⁺

### Step D

To a stirred solution of tert-butyl 2-((5-(dimethylamino)-2-(2,6-dioxopiperidin-3-yl)-1-oxoisoindolin-4-yl)oxy)acetate (40 mg, 0.096 mmol) in DCM (5 mL) was added TFA (3 mL) at 0° C. dropwise to the reaction mixture. Then, the reaction mixture was allowed to stir at ambient temperature for 16 h under nitrogen. After complete consumption of the starting material (monitored by TLC and LCMS) the volatiles were evaporated under reduced pressure. Crude compound was purified by triturating with diethyl ether and n-pentane to get 2-((5-(dimethylamino)-2-(2,6-dioxopiperidin-3-yl)-1-oxoisoindolin-4-yl)oxy)acetic acid (26 mg, 0.072 mmol, 75%) as off white solid.

LCMS (ESI): 362.3 m/z [M+H]⁺

### Step E

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (17.0 mg, 0.025 mmol) and 2-((5-(dimethylamino)-2-(2,6-dioxopiperidin-3-yl)-1-oxoisoindolin-4-yl)oxy)acetic acid (10.0 mg, 0.028 mmol) were dissolved under argon atmosphere in dry DMF (1.0 mL) and DIPEA (0.013 mL, 0.076 mmol), followed by HATU (10.1 mg, 0.026 mmol) were added. Reaction (monitored with LCMS) was stirred for 40 min under argon at room temperature. After complete consumption of the starting material the mixture was diluted with DCM and washed with brine (2×10 mL) and water (2×10 mL). Organic layer was dried over anhydrous magnesium sulphate, filtered and dried under reduced pressure to give crude tert-butyl 6-chloro-1-{2-[4-(2-{[5-(dimethyl amino)-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil, that was directly used in the next step without further purification.

LCMS (ESI): 1017.2 m/z [M+H]⁺

### Step F

Crude tert-butyl 6-chloro-1-(2-(4-(2-((5-(dimethylamino)-2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate from the previous step was dissolved in dry DCM (0.500 mL) under argon atmosphere and TFA (0.500 mL, 6.529 mmol) was added. Reaction (monitored with LCMS) was stirred under argon for 16 h. After complete consumption of the starting material the solution was concentrated under reduced pressure and the resulting residue purified with preparative HPLC (C18, H₂O:MeCN+0.1% FA) to give 6-chloro-1-(2-(4-(2-((5-(dimethylamino)-2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (6.9 mg, 0.007 mmol, 28% over 2 steps) as white solid.

LCMS (ESI): 961.2 m/z [M+H]⁺

¹H NMR (500 MHz, DMSO, 353 K) δ 10.65 (s, 1H), 8.24 (dd, J=9.2, 5.9 Hz, 1H), 7.69 (d, J=8.6 Hz, 1H), 7.58 (dd, J=10.3, 2.6 Hz, 1H), 7.44-7.39 (m, 2H), 7.36 (d, J=8.1 Hz, 1H), 7.32 (ddd, J=9.2, 8.6, 2.6 Hz, 1H), 7.20 (d, J=8.5 Hz, 1H), 7.05 (d, J=8.2 Hz, 1H), 6.87 (dd, J=5.7, 3.0 Hz, 1H), 4.99 (ddd, J=13.0, 5.2, 1.0 Hz, 1H), 4.76 (dd, J=13.9, 2.7 Hz, 1H), 4.72 (dd, J=13.8, 3.4 Hz, 1H), 4.46 (dd, J=16.7, 2.6 Hz, 1H), 4.36 (dd, J=16.6, 3.6 Hz, 1H), 4.31-4.21 (m, 3H), 4.21-4.13 (m, 1H), 3.75 (d, J=1.0 Hz, 3H), 3.37-3.29 (m, 4H), 3.29-3.23 (m, 2H), 2.92-2.82 (m, 7H), 2.67-2.58 (m, 1H), 2.45-2.34 (m, 1H), 2.27-2.19 (m, 2H), 2.16-1.98 (m, 10H), 1.88 (s, 3H).

### Example 9: 6-Chloro-1-(2-(4-(2-((2-(2,6-dioxopiperidin-3-yl)-6-(methylamino)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 12)

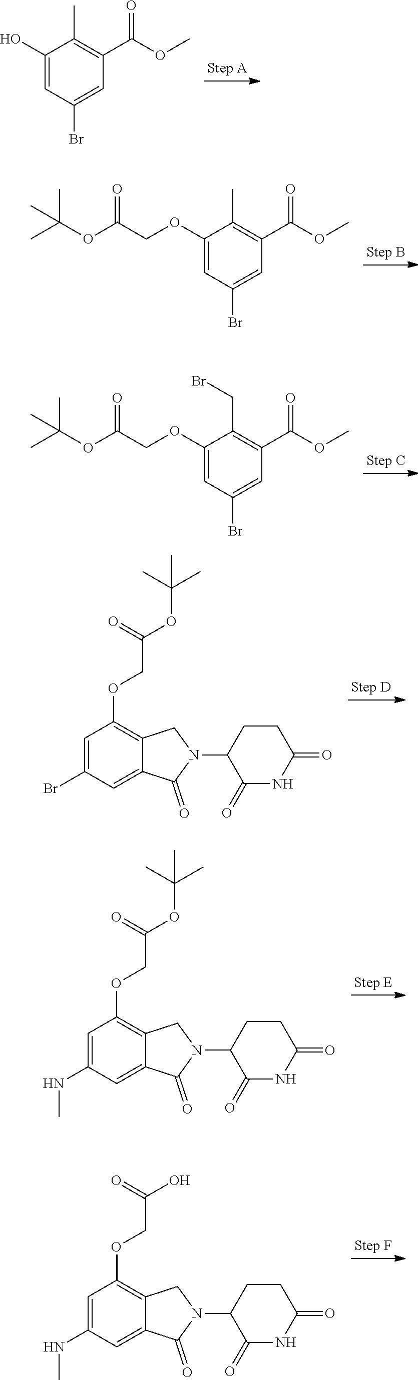

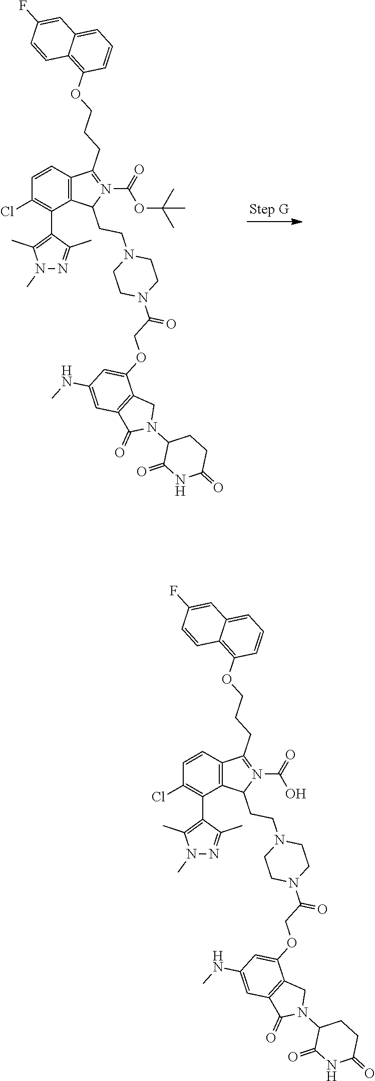

### Step A

To a solution of methyl 5-bromo-3-hydroxy-2-methylbenzoate (8 g, 32.78 mmol) in DMF (150 mL) was added K₂CO₃ (5 g, 36.066 mmol) under nitrogen atmosphere at 0° C. Then tert-butyl 2-bromoacetate (10 g, 51.48 mmol) was added into reaction mixture at RT and the reaction mixture was allowed to stir at RT for 16 h under nitrogen. After complete consumption of the starting material (monitored by TLC and LCMS) the reaction mixture was diluted with ethyl acetate and washed with cold water and brine. The organic layer was dried over anhydrous sodium sulphate and evaporated under reduced pressure to get the crude compound, which was then purified by column chromatography (SiO₂, 30% EtOAc in hexane) to get methyl 5-bromo-3-(2-(tert-butoxy)-2-oxoethoxy)-2-methylbenzoate (6 g, 16.75 mmol, 51%) as white sticky solid.

LCMS (ESI): 304.5 m/z [M-tBu+H]⁺

### Step B

To a solution of methyl 5-bromo-3-(2-(tert-butoxy)-2-oxoethoxy)-2-methylbenzoate (6 g, 16.75 mmol) in dry CCl₄ (100 mL) was added NBS (3.5 g, 20.1 mmol) followed by AIBN (0.55 g, 3.35 mmol) at 0° C. under nitrogen. The reaction mixture was then allowed to stir at 70° C. for 3 h. After complete consumption of the starting material (monitored by TLC and LCMS), the reaction mixture was diluted with EtOAc and washed with water and brine. The organic layer was dried over anhydrous Na₂SO₄ and evaporated under reduced pressure to get 6 g of crude methyl 5-bromo-2-(bromomethyl)-3-(2-(tert-butoxy)-2-oxoethoxy)benzoate which was then used for the next step without further purification.

### Step C

To a solution of methyl 5-bromo-2-(bromomethyl)-3-(2-(tert-butoxy)-2-oxoethoxy)benzoate (6 g, 13.761 mmol) in MeCN (70 mL) was added 3-aminopiperidine-2,6-dione (3 g, 17.89 mmol) followed by DIPEA (7.2 mL, 41.284 mmol) under nitrogen atmosphere. The reaction mixture was stirred at 70° C. for 16 h. The reaction was monitored with LCMS and TLC. After complete consumption of the starting material reaction mass was concentrated and crude material was purified with flash column chromatography (SiO₂, 70% EtOAc in DCM) to get tert-butyl 2-((6-bromo-2-(2,6-dioxopiperidin-3-yl)-1-oxoisoindolin-4-yl)oxy)acetate (3.5 g, 7.74 mmol, 46% over two steps) as black solid.

LCMS (ESI): 454.9 m/z [M+H]⁺

### Step D

To a solution of tert-butyl 2-((6-bromo-2-(2,6-dioxopiperidin-3-yl)-1-oxoisoindolin-4-yl)oxy)acetate (200 mg, 0.442 mmol) in dioxane (8 mL) was added methyl amine (1(M) in THF, 9 mL, 8.85 mmol) followed by Cs₂CO₃ (360 mg, 1.106 mmol). The mixture was deoxygenated with argon and to it Pd-PEPPSI-IHeptCl (172 mg, 0.177 mmol) was added under argon atmosphere. Then the reaction mixture was heated under reflux for 16 h. After complete consumption of the starting material (monitored by TLC and LCMS) the reaction mixture was filtered through celite pad and the solvents were evaporated under reduced pressure get crude tert-butyl 2-((2-(2,6-dioxopiperidin-3-yl)-6-(methylamino)-1-oxoisoindolin-4-yl)oxy)acetate, which was used for the next step without further purification.

LCMS (ESI): 404.1 m/z [M+H]⁺

### Step E

To a solution of tert-butyl 2-((2-(2,6-dioxopiperidin-3-yl)-6-(methylamino)-1-oxoisoindolin-4-yl)oxy)acetate (175 mg, 0.434 mmol) in DCM (10 mL) was added TFA (2 mL) at 0° C. dropwise to the solution and the reaction mixture was allowed to stir at ambient temperature for 16 h under nitrogen. After complete consumption of the starting material (monitored by TLC and LCMS) the volatiles were evaporated under reduced pressure to get the crude compound was purified by preparative HPLC (C18, H₂O:MeCN+0.1% FA) get 2-{[2-(2,6-dioxopiperidin-3-yl)-6-(methylamino)-1-oxo-2,3-dihydro-1H-isoindol-4-yl]oxy}acetic acid (18 mg, 12% over two steps, 0.0518 mmol) as brown solid.

LCMS (ESI): 348.2 m/z [M+H]⁺

### Step F

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (13.0 mg, 0.019 mmol) and 2-{[2-(2,6-dioxopiperidin-3-yl)-6-(methylamino)-1-oxo-2,3-dihydro-1H-isoindol-4-yl]oxy}acetic acid (7.4 mg, 0.021 mmol) were dissolved in dry DMF (0.6 mL) and DIPEA (0.010 mL, 0.058 mmol) was added, followed by HATU (7.7 mg, 0.020 mmol) as a solution in DMF (0.6 mL). Reaction (monitored with LCMS) was stirred at room temperature for 1 h. After complete consumption of the starting material the solution was diluted with DCM up to 10 mL and washed with brine and water. Organic layer was dried over anhydrous sodium sulphate, filtered and concentrated under reduced pressure. Crude tert-Butyl 6-chloro-1-{2-[4-(2-{[2-(2,6-dioxopiperidin-3-yl)-6-(methylamino)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (22.5 mg) was obtained as yellow oil and submitted to the next step without further purification.

LCMS (ESI): 1003.2 m/z [M+H]⁺

### Step G

Crude tert-butyl 6-chloro-1-{2-[4-(2-{[2-(2,6-dioxopiperidin-3-yl)-6-(methylamino)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate was dissolved in dry DCM (0.500 mL) under argon atmosphere and TFA (0.500 mL, 6.529 mmol) was added. Reaction (monitored with LCMS) was stirred for 16 h at room temperature. After complete consumption of the starting material the solution was concentrated under reduced pressure. The resulting residue was dissolved in DMSO and purified with preparative HPLC (C18, H₂O:MeCN+0.1% FA) to give 6-chloro-1-(2-(4-(2-((2-(2,6-dioxopiperidin-3-yl)-6-(methylamino)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (7.0 mg, 0.007 mmol, 53.8% over 2 steps) as white solid.

LCMS (ESI): 947.2 m/z [M+H]⁺

¹H NMR (500 MHz, DMSO, 353 K) δ 10.63 (s, 1H), 8.24 (dd, J=9.2, 5.9 Hz, 1H), 7.70 (d, J=8.5 Hz, 1H), 7.59 (dd, J=10.4, 2.6 Hz, 1H), 7.44-7.39 (m, 2H), 7.33 (ddd, J=9.3, 8.7, 2.7 Hz, 1H), 7.21 (d, J=8.5 Hz, 1H), 6.87 (dd, J=5.8, 2.9 Hz, 1H), 6.46 (d, J=1.7 Hz, 1H), 6.35 (d, J=1.8 Hz, 1H), 4.99 (ddd, J=13.1, 5.2, 1.1 Hz, 1H), 4.77 (s, 2H), 4.34-4.11 (m, 6H), 3.75 (d, J=1.9 Hz, 3H), 3.40-3.33 (m, 4H), 3.31-3.25 (m, 2H), 2.86 (ddd, J=17.4, 13.4, 5.5 Hz, 1H), 2.73 (s, 3H), 2.64-2.57 (m, 1H), 2.44-2.35 (m, 1H), 2.28-2.20 (m, 2H), 2.16-1.97 (m, 101H), 1.88 (s, 3H).

### Example 10: 1-{2-[4-(2-{[6-Amino-2-(2,6-dioxopiperidin-3-yl)-1-oxo-2,3-dihydro-1H-isoindol-4-yl]oxy}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (Compound 11)

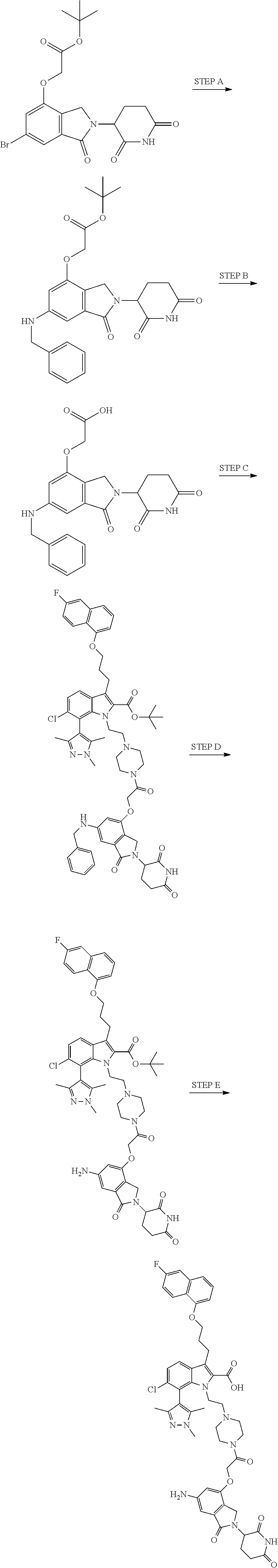

### Step A

To a solution of tert-butyl 2-((6-bromo-2-(2,6-dioxopiperidin-3-yl)-1-oxoisoindolin-4-yl)oxy)acetate (200 mg, 0.44 mmol) in dioxane (8 mL) were added benzyl amine (0.1 ml, 0.885 mmol) and Cs₂CO₃ (360 mg, 1.106 mmol). The mixture was deoxygenated with argon and to it was added Pd-PEPPSI-IHeptCl (129 mg, 0.133 mmol) under argon atmosphere. The reaction mixture was then heated under reflux for 16 h. After complete consumption of the starting material, (monitored by TLC and LCMS), the reaction mixture was filtered through celite pad and the solvents were evaporated under reduced pressure get the crude tert-butyl 2-((6-(benzylamino)-2-(2,6-dioxopiperidin-3-yl)-1-oxoisoindolin-4-yl)oxy)acetate, which was used in the next step without further purification.

LCMS (ESI): 480.4 m/z [M+H]⁺

### Step B

To a solution of tert-butyl 2-((6-(benzylamino)-2-(2,6-dioxopiperidin-3-yl)-1-oxoisoindolin-4-yl)oxy)acetate (200 mg, crude) in DCM (10 mL) was added TFA (2 mL) at 0° C. dropwise, and the reaction mixture was allowed to stir at ambient temperature for 16 h under nitrogen. After complete consumption of the starting material, (monitored by TLC and LCMS), the volatiles were evaporated under reduced pressure to get the crude compound which was then purified by preparative HPLC (C18, 10 mM ammonium acetate in water:MeCN) to get 2-((6-(benzylamino)-2-(2,6-dioxopiperidin-3-yl)-1-oxoisoindolin-4-yl)oxy)acetic acid (55 mg, 0.13 mmol, 29% over two steps) as off white solid.

LCMS (ESI): 424.3 m/z [M+H]⁺

### Step C

To a solution of 2-{[6-(benzylamino)-2-(2,6-dioxopiperidin-3-yl)-1-oxo-2,3-dihydro-1H-isoindol-4-yl]oxy}acetic acid (18.8 mg, 0.044 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (25.0 mg, 0.037 mmol) and HATU (21.1 mg, 0.056 mmol) in dry DMF (2.0 mL) was added DIPEA (0.019 mL, 0.111 mmol).

The mixture was stirred at room temperature for 30 min. The crude was diluted with DCM and washed with brine. The organic layer was dried over Na₂SO₄, filtered and concentrated in vacuo to get 55.2 mg crude tert-butyl 1-{2-[4-(2-{[6-(benzylamino)-2-(2,6-dioxopiperidin-3-yl)-1-oxo-2,3-dihydro-1H-isoindol-4-yl]oxy}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate as yellow oil which was used in next step without further purification.

LCMS (ESI): 1079.4 m/z [M+H]⁺

### Step D

To a solution of tert-butyl 1-{2-[4-(2-{[6-(benzylamino)-2-(2,6-dioxopiperidin-3-yl)-1-oxo-2,3-dihydro-1H-isoindol-4-yl]oxy}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (55.2 mg, crude) in EtOH (10.0 mL) was added Pd/C (10 mg) and the reaction mixture was degassed with argon for 15 min. Hydrogen was bubbled then through reaction mixture for 1 day at room temperature. The reaction mixture was filtered, concentrated under reduced pressure, dissolved in DMSO and purified by preparative HPLC (C18, H₂O:MeCN+0.1% FA) to obtain tert-butyl 1-{2-[4-(2-{[6-amino-2-(2,6-dioxopiperidin-3-yl)-1-oxo-2,3-dihydro-1H-isoindol-4-yl]oxy}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (38.2 mg, crude).

LCMS (ESI): 989.5 m/z [M+H]⁺

### Step E

To a solution of tert-butyl 1-{2-[4-(2-{[6-amino-2-(2,6-dioxopiperidin-3-yl)-1-oxo-2,3-dihydro-1H-isoindol-4-yl]oxy}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylate (38.2 mg, crude) in DCM (1.0 mL) was added TFA (1.0 mL, 13.059 mmol). The mixture was stirred for 18 h at room temperature. The crude was concentrated in vacuo, dissolved in DMSO and purified by preparative HPLC (C18, H₂O:MeCN+0.1% FA) to obtain 1-{2-[4-(2-{[6-amino-2-(2,6-dioxopiperidin-3-yl)-1-oxo-2,3-dihydro-1H-isoindol-4-yl]oxy}acetyl)piperazin-1-yl]ethyl}-6-chloro-3-{3-[(6-fluoronaphthalen-1-yl)oxy]propyl}-7-(1,3,5-trimethyl-1H-pyrazol-4-yl)-1H-indole-2-carboxylic acid (9.5 mg, 0.010 mmol, 27% over three steps) as white solid.

LCMS (ESI): 933.2 m/z [M+H]⁺

¹H NMR (500 MHz, DMSO, 353 K) δ 10.60 (s, 1H), 8.24 (dd, J=9.2, 5.9 Hz, 1H), 7.69 (d, J=8.5 Hz, 1H), 7.58 (dd, J=10.4, 2.6 Hz, 1H), 7.44-7.40 (m, 2H), 7.32 (td, J=8.9, 2.6 Hz, 1H), 7.20 (d, J=8.5 Hz, 1H), 6.87 (dd, J=5.9, 2.7 Hz, 1H), 6.56 (d, J=1.7 Hz, 1H), 6.39 (d, J=1.7 Hz, 1H), 4.97 (ddd, J=13.0, 5.2, 1.2 Hz, 1H), 4.73 (s, 2H), 4.32-4.17 (m, 5H), 4.14 (dd, J=16.2, 2.1 Hz, 1H), 3.75 (d, J=1.8 Hz, 3H), 3.39-3.33 (m, 4H), 3.30-3.25 (m, 2H), 2.89-2.80 (m, 1H), 2.65-2.57 (m, 1H), 2.44-2.34 (m, 1H), 2.27-2.20 (m, 2H), 2.16-2.06 (m, 6H), 2.05-1.98 (m, 4H), 1.88 (s, 3H).

### Example 11: 6-Chloro-1-{2-[4-(2-{[6-(dimethylamino)-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 13)

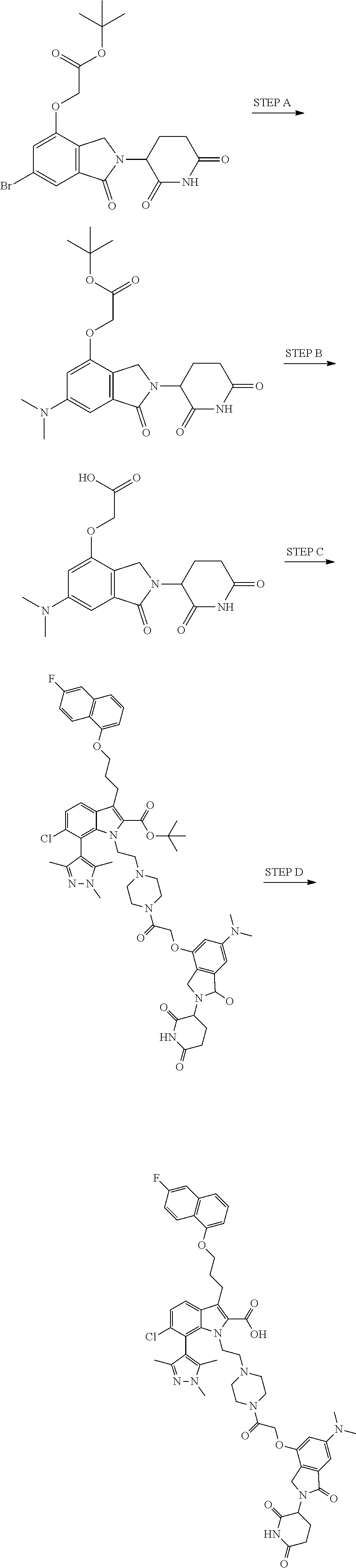

### Step A

To a solution of tert-butyl 2-((6-bromo-2-(2,6-dioxopiperidin-3-yl)-1-oxoisoindolin-4-yl)oxy)acetate (200 mg, 0.44 mmol) in dioxane (8 mL) was added dimethyl amine (1(M) in THF; 9 mL, 8.85 mmol), followed by Cs₂CO₃ (360 mg, 1.106 mmol). The mixture was deoxygenated with argon and to it was added Pd-PEPPSI-IHeptCl (129 mg, 0.133 mmol) under argon atmosphere. Then the reaction mixture was heated under reflux for 16 h. After complete consumption of the starting material, (monitored by TLC and LCMS), the reaction mixture was filtered through celite pad and the solvents were evaporated under reduced pressure get the crude tert-butyl 2-((6-(dimethylamino)-2-(2,6-dioxopiperidin-3-yl)-1-oxoisoindolin-4-yl)oxy)acetate. It was used for the next step without further purification.

LCMS (ESI): 418.2 m/z [M+H]⁺

### Step B

To a solution of tert-butyl 2-((6-(dimethylamino)-2-(2,6-dioxopiperidin-3-yl)-1-oxoisoindolin-4-yl)oxy)acetate (180 mg, crude) in DCM (10 mL) was added dropwise TFA (2 mL) at 0° C. The reaction mixture was allowed to stir at ambient temperature for 16 h under nitrogen. After complete consumption of the starting material, (monitored by TLC and LCMS), the volatiles were evaporated under reduced pressure to get the crude compound which was then purified by preparative HPLC (C18, H₂O:MeCN+0.1% FA) to get 2-((6-(dimethylamino)-2-(2,6-dioxopiperidin-3-yl)-1-oxoisoindolin-4-yl)oxy)acetic acid (17 mg, 0.047 mmol, 11% over two steps) of the target molecule as off white solid.

LCMS (ESI): 362.3 m/z [M+H]⁺

### Step C

To a solution of 2-{[6-(dimethylamino)-2-(2,6-dioxopiperidin-3-yl)-1-oxo-2,3-dihydro-1H-isoindol-4-yl]oxy}acetic acid (6.4 mg, 0.018 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (10.0 mg, 0.015 mmol) and HATU (8.5 mg, 0.022 mmol) in dry DMF (2.0 mL), DIPEA (0.008 mL, 0.044 mmol) was added. The mixture was stirred at room temperature for 30 min. The crude was diluted with DCM and washed with brine. The organic layer was dried over Na₂SO₄, filtered and concentrated in vacuo to get 21.8 mg crude with tert-butyl 6-chloro-1-{2-[4-(2-{[6-(dimethylamino)-2-(2,6-dioxopiperidin-3-yl)-1-oxo-2,3-dihydro-1H-isoindol-4-yl]oxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylate as yellow oil which was used in next step without further purification.

LCMS (ESI): 1017.1 m/z [M+H]⁺

### Step D

To a solution of tert-butyl 6-chloro-1-{2-[4-(2-{[6-(dimethylamino)-2-(2,6-dioxopiperidin-3-yl)-1-oxo-2,3-dihydro-1H-isoindol-4-yl]oxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylate (21.8 mg, crude) in DCM (1.0 mL) was added TFA (1.0 mL, 13.059 mmol). The mixture was stirred for 18 h at RT. The crude was concentrated in vacuo and purified by preparative HPLC (C18, H₂O:MeCN+0.1% FA) to obtain 6-chloro-1-{2-[4-(2-{[6-(dimethylamino)-2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (6.5 mg, 0.007 mmol, 47% over two steps) as white solid.

LCMS (ESI): 961.2 m/z [M+H]⁺

¹H NMR (600 MHz, DMSO, 353 K) δ 10.68 (s, 1H), 8.24 (dd, J=9.2, 5.9 Hz, 1H), 7.69 (d, J=8.5 Hz, 1H), 7.59 (dd, J=10.4, 2.6 Hz, 1H), 7.43-7.39 (m, 2H), 7.33 (ddd, J=9.2, 8.6, 2.6 Hz, 1H), 7.20 (d, J=8.5 Hz, 1H), 6.87 (dd, J=5.8, 2.8 Hz, 1H), 6.60 (d, J=2.0 Hz, 1H), 6.49 (d, J=2.0 Hz, 1H), 5.02 (dd, J=13.8, 5.0 Hz, 1H), 4.84 (s, 2H), 4.31-4.25 (m, 2H), 4.24 (t, J=6.3 Hz, 2H), 4.21-4.15 (m, 2H), 3.74 (d, J=3.0 Hz, 3H), 3.40-3.34 (m, 4H), 3.29-3.24 (m, 2H), 2.94 (d, J=0.7 Hz, 6H), 2.87 (ddd, J=17.3, 13.5, 5.5 Hz, 1H), 2.63-2.57 (m, 1H), 2.44-2.37 (m, 1H), 2.26-2.20 (m, 2H), 2.13-2.01 (m, 7H), 2.00 (s, 3H), 1.88 (s, 3H).

### Example 12: 6-Chloro-1-{2-[4-(2-{[2-(2,6-dioxopiperidin-3-yl)-1-oxo-6-(piperidin-1-yl)-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 14)

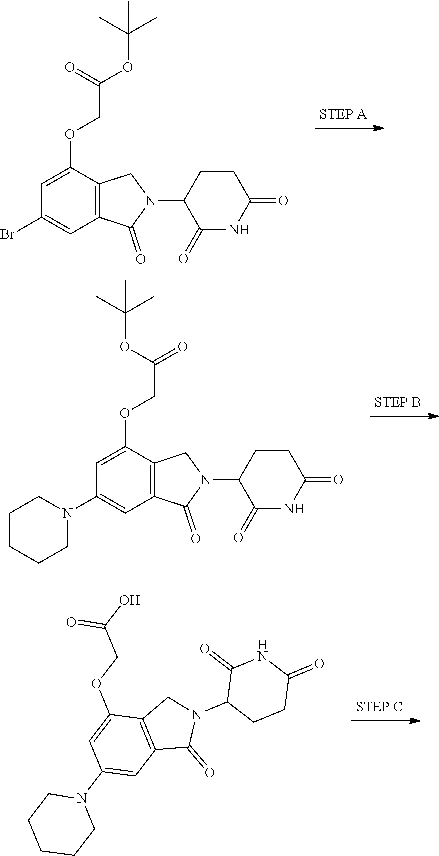

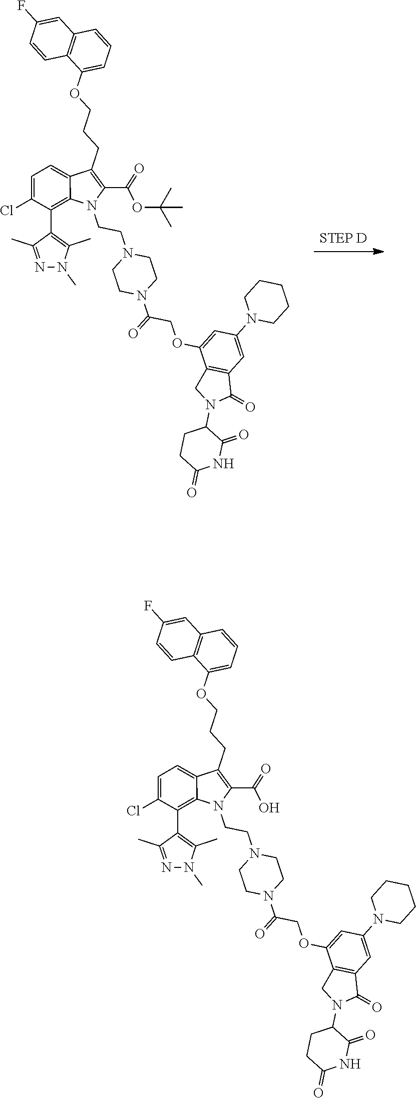 [SMILES: Cc1nn(C)c(C)c1-c1c(Cl)ccc2c(CCCOc3cccc4cc(F)ccc34)c(C(=O)OC(C)(C)C)n(CCN3CCN(C(=O)COc4cc(N5CCCCC5)cc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cc(N5CCCCC5)cc5c4CN(C4CCC(=O)NC4=O)C5=O)CC3)c12]

### Step A

To a solution of tert-butyl 2-((6-bromo-2-(2,6-dioxopiperidin-3-yl)-1-oxoisoindolin-4-yl)oxy)acetate (200 mg, 0.44 mmol) in dioxane (8 mL) was added piperidine (0.1 mL, 0.885 mmol), followed by Cs₂CO₃ (360 mg, 1.106 mmol). The mixture was deoxygenated with argon and to it was added Pd-PEPPSI-IHeptCl (86 mg, 0.088 mmol) under argon atmosphere. Then, the reaction mixture was heated under reflux for 16 h. After complete consumption of the starting material, (monitored by TLC and LCMS), the reaction mixture was filtered through celite pad and the solvents were evaporated under reduced pressure get the crude tert-butyl 2-((2-(2,6-dioxopiperidin-3-yl)-1-oxo-6-(piperidin-1-yl)isoindolin-4-yl)oxy)acetate. It was used for the next step without further purification.

LCMS (ESI): 458.0 m/z [M+H]⁺

### Step B

To a solution of tert-butyl 2-((2-(2,6-dioxopiperidin-3-yl)-1-oxo-6-(piperidin-1-yl)isoindolin-4-yl)oxy)acetate (200 mg, crude) in DCM (10 mL) was added dropwise TFA (2 mL) at 0° C. The reaction mixture was allowed to stir at ambient temperature for 16 h under nitrogen. After complete consumption of the starting material, (monitored by TLC and LCMS), the volatiles were evaporated under reduced pressure to get the crude compound which was then purified by preparative HPLC (C18, 10 mM ammonium acetate in water:MeCN) to get 2-((2-(2,6-dioxopiperidin-3-yl)-1-oxo-6-(piperidin-1-yl)isoindolin-4-yl)oxy)acetic acid (44 mg, 0.11 mmol, 25% over two steps) as off white solid.

LCMS (ESI): 402.3 m/z [M+H]⁺

### Step C

To a solution of 2-{[2-(2,6-dioxopiperidin-3-yl)-1-oxo-6-(piperidin-1-yl)-2,3-dihydro-1H-isoindol-4-yl]oxy}acetic acid (10.7 mg, 0.027 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15.0 mg, 0.022 mmol) and HATU (12.7 mg, 0.033 mmol) in dry DMF (2.0 mL) was added DIPEA (0.012 mL, 0.067 mmol). The mixture was stirred at room temperature for 30 min. The crude was diluted with DCM and washed with brine. The organic layer was dried over Na₂SO₄, filtered and concentrated in vacuo to get 35.8 mg crude tert-butyl 6-chloro-1-{2-[4-(2-{[2-(2,6-dioxopiperidin-3-yl)-1-oxo-6-(piperidin-1-yl)-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used in next step without further purification.

LCMS (ESI): 1057.6 m/z [M+H]⁺

### Step D

To a solution of tert-butyl 6-chloro-1-{2-[4-(2-{[2-(2,6-dioxopiperidin-3-yl)-1-oxo-6-(piperidin-1-yl)-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (35.8 mg, crude) in DCM (0.3 mL) was added TFA (0.3 mL). The mixture was stirred for 18 h at RT. The crude was concentrated in vacuo and purified by preparative HPLC (C18, H₂O:MeCN+0.1% FA) to obtain 6-chloro-1-{2-[4-(2-{[2-(2,6-dioxopiperidin-3-yl)-1-oxo-6-(piperidin-1-yl)-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (6.5 mg, 0.006 mmol, 22% over two steps) as a white solid.

LCMS (ESI): 1001.2 m/z [M+H]⁺

1H NMR (600 MHz, DMSO, 353 K) δ 10.68 (s, 1H), 8.24 (dd, J=9.2, 5.9 Hz, 1H), 7.70 (d, J=8.5 Hz, 1H), 7.59 (dd, J=10.3, 2.6 Hz, 1H), 7.44-7.39 (m, 2H), 7.33 (ddd, J=9.3, 8.6, 2.6 Hz, 1H), 7.21 (d, J=8.5 Hz, 1H), 6.87 (dd, J=5.9, 2.7 Hz, 1H), 6.77 (d, J=1.9 Hz, 1H), 6.71-6.68 (m, 1H), 5.02 (dd, J=13.0, 5.2 Hz, 1H), 4.84 (s, 2H), 4.30-4.15 (m, 6H), 3.75 (d, J=2.5 Hz, 3H), 3.39-3.33 (m, 4H), 3.31-3.25 (m, 2H), 3.21-3.16 (m, 4H), 2.87 (ddd, J=17.4, 13.5, 5.5 Hz, 1H), 2.63-2.58 (m, 1H), 2.45-2.36 (m, 1H), 2.26-2.20 (m, 2H), 2.13-2.00 (m, 101H), 1.88 (s, 3H), 1.65-1.60 (m, 4H), 1.59-1.54 (m, 2H).

### Example 13: 6-Chloro-1-{2-[4-(2-{[2-(2,6-dioxopiperidin-3-yl)-3-methyl-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 15)

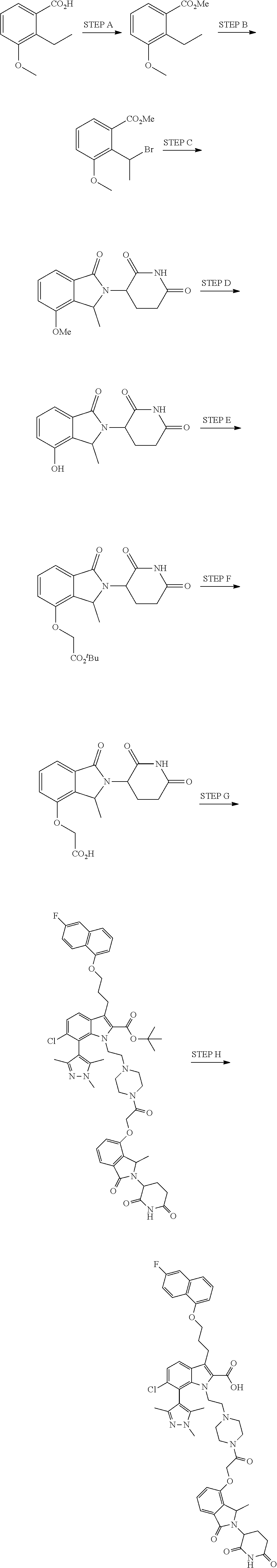

### Step A

To a solution of 2-ethyl-3-methoxybenzoic acid (4.23 g, 23.5 mmol) in MeOH (120 mL) was added sulfuric acid (2.00 mL, 37.6 mmol) and the reaction was stirred for 16 hours under reflux. The reaction was cooled down to room temperature and the solvent was removed in vacuo. The residue was dissolved with Et₂O, sat. aq. NaHCO₃ solution was added then water. The layers were separated, and the aqueous layer was extracted twice with Et₂O. Combined organic layers were washed with brine, dried over Na₂SO₄ and concentrated in vacuo. The crude residue was eluted with mixture of cyclohexane/Et₂O (3:2). The filtrate was concentrated to obtain methyl 2-ethyl-3-methoxybenzoate (3.67 g, 18.9 mmol, 80%) as a colorless oil.

### Step B

Methyl 2-ethyl-3-methoxybenzoate (2.67 g, 13.7 mmol) was dissolved in EtOAc (55.0 mL), N-bromosuccinimide (2.81 g, 15.8 mmol) and benzoyl peroxide (166 mg, 0.69 mmol) were sequentially added. The reaction mixture under reflux for 16 hours. The reaction was cooled down to room temperature and sat. aq. NaHCO₃ solution was added. The layers were separated, the aqueous layer was extracted twice with EtOAc. The combined organic layers were washed with brine, dried over Na₂SO₄ and concentrated in vacuo. The crude material of methyl 2-(1-bromoethyl)-3-methoxybenzoate was used to the next step without further purification.

### Step C

An impure fraction containing methyl 2-(1-bromoethyl)-3-methoxybenzoate (500 mg, crude) was dissolved in MeCN (9.2 mL). DIPEA (957 mL, 5.49 mmol) was added followed by 3-aminopiperidine-2,6-dione hydrochloride (452.0 mg). The resulting solution was stirred under reflux for 14 hours. The reaction was cooled down to room temperature and the solvent was removed in vacuo. The residue was dissolved in EtOAc and aq. 1M HCl was added. The layers were separated, and the aqueous layer was extracted twice with EtOAc. Combined organic layers were washed with brine, dried over Na₂SO₄ and concentrated in reduced pressure. The crude was purified by flash column chromatography (SiO₂, 0-20% EtOAc in cyclohexane) to obtain impurity fraction of 3-(4-methoxy-3-methyl-1-oxoisoindolin-2-yl)piperidine-2,6-dione.

### Step D

An impure fraction containing 3-(4-methoxy-3-methyl-1-oxoisoindolin-2-yl)piperidine-2,6-dione (40.0 mg, crude) was dissolved in DCM (1.4 mL) and cooled down to 0° C. BBr₃ (1.0M in DCM, 832 mL, 832 mmol) was added dropwise and the reaction was stirred at room temperature for 2 hours. At 0° C., sat. aq. NaHCO₃ was added dropwise and diluted with DCM. The layers were separated, and the aqueous layer was extracted twice with DCM. Combined organic layers were washed with brine, dried over Na₂SO₄ and concentrated in vacuo. The crude of 3-(4-hydroxy-3-methyl-1-oxoisoindolin-2-yl)piperidine-2,6-dione (38.0 mg) was directly engaged in the next without further purification.

### Step E

3-(4-hydroxy-3-methyl-1-oxoisoindolin-2-yl)piperidine-2,6-dione (38.0 mg, crude) was dissolved in DMF (1.4 mL). KHCO₃ (28.0 mg, 278 mmol) and KI (11.5 mg, 69.5 mmol) were sequentially added. tert-Butyl bromoacetate (31 mL, 208 mmol) was then added and the reaction was heated to 70° C. for 12 h. The reaction was cooled down to room temperature, water was added followed by EtOAc. The layers were separated, the organic layer was washed three times with water then three times with brine. The organic fraction was dried over Na₂SO₄ and concentrated in vacuo. The crude tert-butyl 2-((2-(2,6-dioxopiperidin-3-yl)-3-methyl-1-oxoisoindolin-4-yl)oxy)acetate was directly engaged in the next step without further purification.

### Step F

To a solution of tert-butyl 2-((2-(2,6-dioxopiperidin-3-yl)-3-methyl-1-oxoisoindolin-4-yl)oxy)acetate (54.0 mg, crude) in DCM (2.8 mL) at 0° C. was added TFA (0.428 mL, 3.60 mmol). The mixture was allowed to warm up to room temperature and stirred for 5 hours. The volatiles were removed in vacuo. The crude residue was dissolved in MeOH and concentrated in vacuo. The operation was repeated three times to remove remaining TFA. The crude residue was purified by reverse phase (C18, H₂O:MeCN+0.1% FA) to obtain the desired product as a 2:1 mixture of diastereomers 2-((2-(2,6-dioxopiperidin-3-yl)-3-methyl-1-oxoisoindolin-4-yl)oxy)acetic acid (11.5 mg, 0.035 mmol, 25% over 3 steps) as a white solid.

LCMS (ESI): 333.0 m/z [M+H]⁺

### Step G

To a solution of 2-((2-(2,6-dioxopiperidin-3-yl)-3-methyl-1-oxoisoindolin-4-yl)oxy)acetic acid (5.0 mg, 0.015 mmol) and tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (8.5 mg, 0.013 mmol) in dry DMF (2.0 mL) were added HATU (9.6 mg, 0.025 mmol) and DIPEA (0.007 mL, 0.038 mmol). The reaction was stirred at RT for 20 min. The crude was diluted in DCM and washed with brine. The organic layer was dried over MgSO₄, filtered and concentrated in vacuo to obtain tert-butyl 6-chloro-1-{2-[4-(2-{[2-(2,6-dioxopiperidin-3-yl)-3-methyl-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (37.5 mg, crude) as an yellow oil, which was used to next step without further purification.

LCMS: (ESI) 989.1 m/z [M+H]⁺

### Step H

To a solution of tert-butyl 6-chloro-1-{2-[4-(2-{[2-(2,6-dioxopiperidin-3-yl)-3-methyl-1-oxo-2,3-dihydro-1H-isoindol-4-yl]oxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylate (37.5 mg, crude) in DCM (1.0 mL) was added TFA (1.0 mL, 13.059 mmol). The reaction mixture was stirred for 18 h at RT. The crude was concentrated in vacuo and purified by preparative HPLC (C18, H₂O:MeCN+0.1% FA) to obtain 6-chloro-1-{2-[4-(2-{[2-(2,6-dioxopiperidin-3-yl)-3-methyl-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (6.7 mg, 0.007 mmol, 19.1%) as yellow solid.

LCMS (ESI): 932.2 m/z [M+H]⁺

¹H NMR (500 MHz, DMSO, 353 K) δ 10.59 (s, 0.6H), 10.56 (s, 0.4H), 8.24 (dd, J=9.3, 5.9 Hz, 1H), 7.71 (d, J=8.5 Hz, 1H), 7.58 (dd, J=10.4, 2.6 Hz, 1H), 7.44-7.39 (m, 3H), 7.32 (td, J=8.9, 2.6 Hz, 1H), 7.28-7.25 (m, 1H), 7.21 (d, J=8.5 Hz, 1H), 7.16-7.12 (m, 1H), 6.87 (dd, J=5.7, 2.9 Hz, 1H), 4.88 (s, 2H), 4.82-4.75 (m, 0.4H), 4.73-4.63 (m, 1.6H), 4.31-4.23 (m, 3H), 4.22-4.16 (m, 1H), 3.76 (s, 3H), 3.40-3.34 (m, 4H), 3.30-3.26 (m, 2H), 2.87-2.55 (m, 3H), 2.27-2.21 (m, 2H), 2.16-2.03 (m, 7H), 2.01 (s, 3H), 1.89 (s, 3H), 1.52 (d, J=6.6 Hz, 1H), 1.50 (d, J=6.6 Hz, 2H).

### Example 14: 6-Chloro-1-(2-(4-(2-((1-(2,6-dioxopiperidin-3-yl)-2-oxo-1,2-dihydrobenzo[cd]indol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 21)

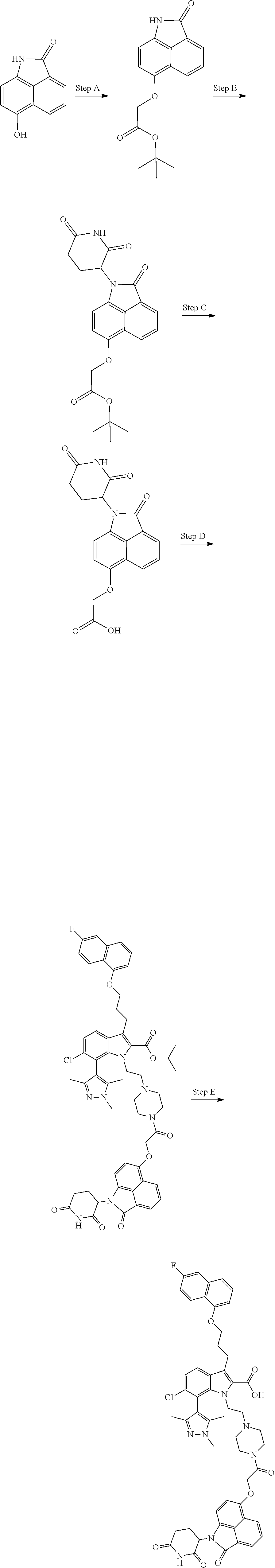 [SMILES: O=C1Nc2ccc(O)c3cccc1c23 ; CC(C)(C)OC(=O)COc1ccc2c3c(cccc13)C(=O)N2 ; CC(C)(C)OC(=O)COc1ccc2c3c(cccc13)C(=O)N2C1CCC(=O)NC1=O ; O=C(O)COc1ccc2c3c(cccc13)C(=O)N2C1CCC(=O)NC1=O ; Cc1nn(C)c(C)c1-c1c(Cl)ccc2c(CCCOc3cccc4cc(F)ccc34)c(C(=O)OC(C)(C)C)n(CCN3CCN(C(=O)COc4ccc5c6c(cccc46)C(=O)N5C4CCC(=O)NC4=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c6c(cccc46)C(=O)N5C4CCC(=O)NC4=O)CC3)c12]

### Step A

9-Hydroxy-2-azatricyclo[6.3.1.0⁴,¹²]dodeca-1(11),4,6,8(12),9-pentaen-3-one (25.1 mg, 0.135 mmol) was dissolved in dry DMF (1.4 mL) under argon atmosphere and Cs₂CO₃ (132.3 mg, 0.406 mmol) was added. Reaction was stirred at room temperature for 10 min and tert-butyl 2-bromoacetate (0.020 mL, 0.135 mmol) was added. Reaction (monitored with LCMS) was stirred at room temperature under argon for 90 min. After complete consumption of the starting material DCM (10 mL) was added and the solution was washed with brine and water. Organic layer was dried over anhydrous magnesium sulphate, filtered and dried under reduced pressure. The resulting residue was dissolved in DMSO and purified with reverse phase preparative HPLC to obtain tert-butyl 2-({3-oxo-2-azatricyclo[6.3.1.0⁴,¹²]dodeca-1(11),4,6,8(12),9-pentaen-9-yl}oxy)acetate (25.3 mg, 0.085 mmol, 62.4%).

LCMS (ESI): 300.1 m/z [M+H]⁺

### Step B

To a well stirred solution of tert-butyl 2-({3-oxo-2-azatricyclo[6.3.1.0⁴,¹²]dodeca-1(11),4,6,8(12),9-pentaen-9-yl}oxy)acetate (25.3 mg, 0.085 mmol) in DMF (2 mL) was added NaH (50.7 mg, 1.268 mmol) portionwise at 0° C. under nitrogen and the reaction mixture was allowed to stir at the same temperature for 30 min. A DMF (2 mL) solution of 3-bromopiperidine-2,6-dione (162.3 mg, 0.845 mmol) was then slowly added dropwise to the reaction mixture at 0° C. and the reaction mass was allowed to stir at the same temperature for 5 minutes. The mixture was then heated to 80° C. and the reaction (monitored with TLC (SiO₂, 70% ethyl acetate in isohexane) was continued for 2 h under nitrogen. The reaction was cooled down to −80° C. and was quenched with solid ammonium chloride. The mixture was allowed to slowly reach room temperature and diluted with DCM. The mixture was filtrated through Schott funnel and the separated solid washed with DCM. The filtrate was concentrated and dried under reduced pressure. Crude product was purified with preparative TLC (SiO₂, 70% EtOAc in isohexane) to give tert-butyl 2-{[2-(2,6-dioxopiperidin-3-yl)-3-oxo-2-azatricyclo[6.3.1.0⁴,¹²]dodeca-1(11),4,6,8(12),9-pentaen-9-yl]oxy}acetate (10.6 mg, 0.026 mmol, 30.4%) as yellow solid.

LCMS (ESI): 411.0 m/z [M+H]⁺

### Step C

tert-Butyl 2-{[2-(2,6-dioxopiperidin-3-yl)-3-oxo-2-azatricyclo[6.3.1.0⁴,¹²]dodeca-1(11),4,6,8(12),9-pentaen-9-yl]oxy}acetate (10.6 mg, 0.026 mmol) was dissolved in dry DCM (0.50 mL) under argon atmosphere and TFA (0.500 mL, 6.529 mmol) was added. Reaction was stirred at room temperature under argon. After 16 h LCMS indicated complete consumption of the starting material. The solution was concentrated to dryness under reduced pressure. The resulting residue was twice dissolved in 1M aqueous HCl (1 mL) and concentrated under vacuum. 2-{[2-(2,6-dioxopiperidin-3-yl)-3-oxo-2-azatricyclo[6.3.1.0⁴,¹²]dodeca-1(11),4,6,8(12),9-pentaen-9-yl]oxy}acetic acid (9.0 mg, 0.025 mmol, 98.8%) was obtained as yellow solid. Reaction product was used in the next step without further purification.

LCMS (ESI): 355.3 m/z [M+H]⁺

### Step D

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (14.3 mg, 0.021 mmol) and 2-{[2-(2,6-dioxopiperidin-3-yl)-3-oxo-2-azatricyclo[6.3.1.0⁴,¹²]dodeca-1(11),4,6,8(12),9-pentaen-9-yl]oxy}acetic acid (9.0 mg, 0.025 mmol) were dissolved in dry DMF (1.0 mL) under argon atmosphere. To it, DIPEA (0.011 mL, 0.064 mmol) was added, followed by dropwise addition of HATU (8.5 mg, 0.022 mmol) as a solution in DMF (1.0 mL). Reaction (monitored with LCMS) was stirred for 20 min at room temperature under argon. After complete consumption of the starting material the solution was diluted with DCM up to 10 mL and washed with brine and water. Organic layer was dried over anhydrous magnesium sulphate, filtrated and dried under reduced pressure to give crude tert-butyl 6-chloro-1-(2-(4-(2-((1-(2,6-dioxopiperidin-3-yl)-2-oxo-1,2-dihydrobenzo[cd]indol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (22.5 mg) as yellow oil. Reaction product was used in the next step without further purification.

LCMS (ESI): 1010.2 m/z [M+H]⁺

### Step E

Crude tert-butyl 6-chloro-1-(2-(4-(2-((1-(2,6-dioxopiperidin-3-yl)-2-oxo-1,2-dihydrobenzo[cd]indol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (22.5 mg) was dissolved in dry DCM (0.50 mL) under argon atmosphere and TFA (0.50 mL) was added. Reaction (monitored with LCMS) was stirred at room temperature under argon for 16 h. After complete consumption of the starting material the solution was concentrated under reduced pressure. Resulting residue was dissolved in DMSO and purified with reverse phase HPLC (C18, H₂O:MeCN+0.1% FA) to give 6-chloro-1-(2-(4-(2-((1-(2,6-dioxopiperidin-3-yl)-2-oxo-1,2-dihydrobenzo[cd]indol-6-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (9.1 mg, 0.010 mmol, 47.6% over 2 steps) as yellow solid.

LCMS (ESI): 954.15 m/z [M+H]⁺

¹H NMR (500 MHz, DMSO, 353 K) δ 10.77 (s, 1H), 8.29 (dd, J=8.2, 0.7 Hz, 1H), 8.24 (dd, J=9.2, 5.9 Hz, 1H), 8.08 (dd, J=7.1, 0.6 Hz, 1H), 7.83 (dd, J=8.2, 7.0 Hz, 1H), 7.69 (d, J=8.5 Hz, 1H), 7.58 (dd, J=10.4, 2.6 Hz, 1H), 7.45-7.37 (m, 2H), 7.32 (ddd, J=9.2, 8.6, 2.6 Hz, 1H), 7.20 (d, J=8.5 Hz, 1H), 6.97 (d, J=7.8 Hz, 1H), 6.91-6.81 (m, 2H), 5.35 (dd, J=12.6, 5.5 Hz, 1H), 4.91 (s, 2H), 4.33-4.22 (m, 3H), 4.18 (ddd, J=13.9, 7.9, 5.8 Hz, 1H), 3.74 (s, 3H), 3.47-3.35 (m, 4H), 3.32-3.23 (m, 2H), 2.93 (ddd, J=16.7, 12.9, 5.4 Hz, 1H), 2.79-2.65 (m, 2H), 2.28-2.20 (m, 2H), 2.18-2.02 (m, 7H), 1.99 (s, 3H), 1.88 (s, 3H).

### Example 15: 6-Chloro-1-{2-[4-(2-{3-[(2,6-dioxopiperidin-3-yl)carbamoyl]-2-ethylphenoxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 24)

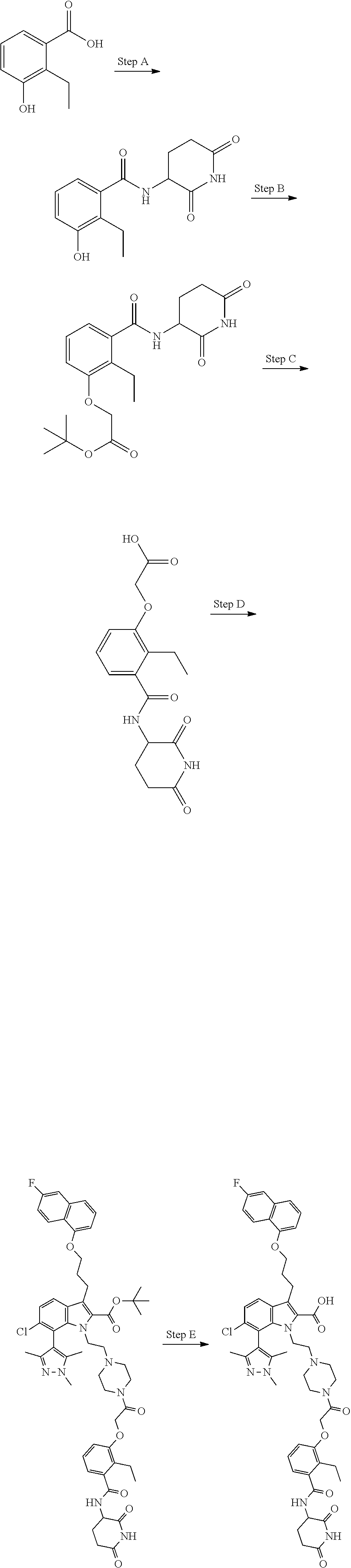 [SMILES: CCc1c(O)cccc1C(=O)O ; CCc1c(O)cccc1C(=O)NC1CCC(=O)NC1=O ; CCc1c(OCC(=O)OC(C)(C)C)cccc1C(=O)NC1CCC(=O)NC1=O ; CCc1c(OCC(=O)O)cccc1C(=O)NC1CCC(=O)NC1=O ; CCc1c(OCC(=O)N2CCN(CCn3c(C(=O)OC(C)(C)C)c(CCCOc4cccc5cc(F)ccc45)c4ccc(Cl)c(-c5c(C)nn(C)c5C)c43)CC2)cccc1C(=O)NC1CCC(=O)NC1=O ; CCc1c(OCC(=O)N2CCN(CCn3c(C(=O)O)c(CCCOc4cccc5cc(F)ccc45)c4ccc(Cl)c(-c5c(C)nn(C)c5C)c43)CC2)cccc1C(=O)NC1CCC(=O)NC1=O]

### Step A

To a well stirred solution of 2-ethyl-3-hydroxybenzoic acid (600 mg, 3.614 mmol) in DMF (12 ml) was added HATU (1.643 g, 4.337 mmol) and DIPEA (1.26 ml, 7.23 mmol) successively at RT under nitrogen. Then the reaction mixture was allowed to stir for 15 min at RT. To the reaction mixture was added 3-aminopiperidine-2,6-dione (873 mg, 5.422 mmol) and then the resulting reaction mixture was stirred for another 1 h at RT. After complete consumption of starting material (monitored by LC-MS) the reaction mixture was quenched by adding ice-water, evaporated under vacuum and diluted with DCM, washed by saturated sodium bi carbonate solution, water and brine, dried over anhydrous Na₂SO₄ and filtered. The filtrate was concentrated to get the crude compound which was purified by column chromatography (SiO₂, 50-60% ethyl acetate in DCM) to get N-(2,6-dioxopiperidin-3-yl)-2-ethyl-3-hydroxybenzamide (400 mg, 1.45 mmol, 40%) as white solid.

LCMS (ESI): 278.8 m/z [M+H]⁺

### Step B

To a well stirred solution of N-(2,6-dioxopiperidin-3-yl)-2-ethyl-3-hydroxybenzamide (400 mg, 1.45 mmol) in DMF (5 ml) was added potassium bicarbonate (435 mg, 4.34 mmol) and tert-butyl bromo acetate (0.213 ml, 1.45 mmol) successively at RT under nitrogen. The reaction mixture was allowed to stir at 60° C. for 1 h. After complete consumption of the starting material the reaction mixture was diluted with ethyl acetate, washed successively with water and brine, the organic layer was dried over sodium sulphate and evaporated under reduced pressure to get the crude compound, which was then purified by by preparative HPLC (10 mM ammonium acetate:ACN) to get (80 mg, 0.204 mmol, 14%) tert-butyl 2-(3-((2,6-dioxopiperidin-3-yl)carbamoyl)-2-ethylphenoxy)acetate as white solid.

LCMS (ESI): 391.27 m/z [M+H]⁺

### Step C

To a stirred solution of tert-butyl 2-(3-((2,6-dioxopiperidin-3-yl)carbamoyl)-2-ethylphenoxy)acetate (70 mg, 0.179 mmol) in dichloromethane (3 ml) was added TFA (1 ml) at 0° C. drop wise to the reaction mixture and the reaction mixture was allowed to stir at ambient temperature for 16 h under nitrogen. After complete consumption of the starting material the volatiles were evaporated under reduced pressure to get the crude compound which was then purified by triturating with diethyl ether and n-pentane to get 2-{3-[(2,6-dioxopiperidin-3-yl)carbamoyl]-2-ethylphenoxy}acetic acid (55 mg, 0.164 mmol, 91.6%) of the as off white solid.

LCMS (ESI): 333.2 m/z [M+H]⁺

### Step D

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol), 2-{3-[(2,6-dioxopiperidin-3-yl)carbamoyl]-2-ethylphenoxy}acetic acid (11.9 mg, 0.036 mmol) and HATU (16.9 mg, 0.044 mmol) in dry DMF (2.0 mL) was added DIPEA (0.026 mL, 0.148 mmol). The mixture was stirred at RT for 30 min. The crude was diluted with DCM and washed with brine. The organic layer was dried over MgSO₄, filtered and concentrated in vacuo to get 38.9 mg crude with tert-butyl 6-chloro-1-{2-[4-(2-{3-[(2,6-dioxopiperidin-3-yl)carbamoyl]-2-ethylphenoxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate as yellow oil which was used in next step without further purification.

LCMS (ESI): 991.0 m/z [M+H]⁺

### Step E

To a solution of tert-butyl 6-chloro-1-{2-[4-(2-{3-[(2,6-dioxopiperidin-3-yl)carbamoyl]-2-ethylphenoxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (38.9 mg, crude) in DCM (1.0 mL) was added TFA (1.0 mL, 13.059 mmol). The mixture was stirred for 18 h at RT. The crude was concentrated in vacuo and purified by preparative HPLC (H₂O:ACN+0.1% FA) to obtain 6-chloro-1-{2-[4-(2-{3-[(2,6-dioxopiperidin-3-yl)carbamoyl]-2-ethylphenoxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylic acid (11.0 mg, 0.012 mmol, 30.0%) as a white solid.

LCMS (ESI): 934.2 m/z [M+H]⁺

¹H NMR (500 MHz, DMSO, 353 K) δ 10.48 (s, 1H), 8.24 (dd, J=9.2, 5.9 Hz, 2H), 7.69 (d, J=8.4 Hz, 1H), 7.58 (dd, J=10.3, 2.8 Hz, 1H), 7.45-7.39 (m, 2H), 7.33 (td, J=8.9, 2.7 Hz, 1H), 7.20 (d, J=8.4 Hz, 1H), 7.16 (t, J=7.9 Hz, 1H), 6.98-6.92 (m, 2H), 6.87 (dd, J=5.8, 2.9 Hz, 1H), 4.75 (s, 2H), 4.69 (ddd, J=11.3, 8.3, 5.6 Hz, 1H), 4.31-4.23 (m, 3H), 4.22-4.15 (m, 1H), 3.76 (s, 3H), 3.41-3.36 (m, 4H), 3.30-3.24 (m, 2H), 2.80-2.71 (m, 3H), 2.61-2.54 (m, 1H), 2.27-2.21 (m, 2H), 2.15-2.04 (m, 8H), 2.01 (s, 3H), 1.89 (s, 3H), 1.12 (t, J=7.3 Hz, 3H).

### Example 16: 6-Chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 22)

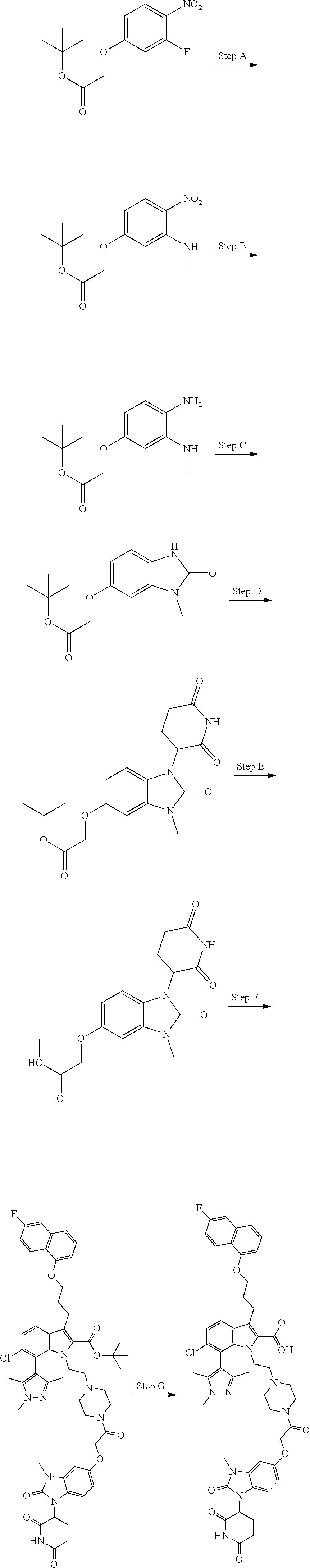

### Step A

To a solution of tert-butyl 2-(3-fluoro-4-nitrophenoxy)acetate (500 mg, 1.844 mmol) in EtOH (10 mL), an aqueous solution of methyl amine (0.41 ml, 9.22 mmol, 40% in H₂O) was added at room temperature and the reaction mixture was stirred for 2 h at 50° C. After complete consumption of the starting material (monitored by TLC and LCMS), the volatiles were removed and crude was purified by flash column chromatography (SiO₂, 50% EtOAc in hexane) to afford tert-butyl 2-(3-(methylamino)-4-nitrophenoxy)acetate (400 mg, 1.418 mmol, 77%) as brown liquid.

LCMS (ESI): 283.2 m/z [M+H]⁺

### Step B

A solution of tert-butyl 2-(3-(methylamino)-4-nitrophenoxy)acetate (400 mg, 1.418 mmol) in MeOH (10 mL) was degassed with argon for 15 minutes. Then, Pd/C (400 mg) was added slowly at room temperature under nitrogen. The reaction mixture was then stirred for 4 h at room temperature under H₂ balloon pressure. After complete consumption of starting material, the reaction mixture was filtered through celite pad and the filtrate was concentrated under reduced pressure to get the crude material. Reaction product was purified with column chromatography (SiO₂, 70% EtOAc in hexane) to get tert-butyl 2-(4-amino-3-(methylamino)phenoxy)acetate (250 mg, 0.992 mmol, 70%) as brown liquid.

LCMS (ESI): 253.1 m/z [M+H]⁺

### Step C

To a solution of tert-butyl 2-(4-amino-3-(methylamino)phenoxy)acetate (250 mg, 0.992 mmol) in THF (6 mL), was added CDI (290 mg, 1.785 mmol) at room temperature under nitrogen atmosphere. The reaction mixture was stirred at 70° C. for 4 h and monitored with LCMS and TLC. After the reaction was complete, volatiles were evaporated and crude material was purified with column chromatography (SiO₂, 50-60% EtOAc in hexane) to afford tert-butyl 2-((3-methyl-2-oxo-2,3-dihydro-1H-benzo[d]imidazol-5-yl)oxy)acetate (150 mg, 0.539 mmol, 55%) as white solid.

LCMS (ESI): 278.9 m/z [M+H]⁺

### Step D

To a solution of tert-butyl 2-((3-methyl-2-oxo-2,3-dihydro-1H-benzo[d]imidazol-5-yl)oxy)acetate (150 mg, 0.54 mmol) in DMF (4 mL) was added NaH (388 mg, 8.1 mmol) portionwise at 0° C. under nitrogen. The reaction mixture was allowed to stir at the same temperature for 30 minutes. DMF (2 mL) solution of 3-bromopiperidine-2,6-dione (1.026 g, 5.4 mmol) was then added dropwise to the reaction at 0° C. and the reaction mixture was allowed to stir at the same temperature for 5 minutes. The mixture was then heated to 80° C. and was stirred for the next 2 h under nitrogen. After complete consumption of the starting material (monitored by LCMS) the reaction mixture was cooled to −78° C., diluted with ethyl acetate and stirred for the next 5 min. A saturated solution of ammonium chloride was then added dropwise to the reaction mixture to quench the excess sodium hydride. Organic layer was separated from the solidified aqueous layer and the solid were washed 3 times with EtOAc. The combined organic layer was washed with brine and evaporated under reduced pressure to get crude tert-butyl 2-((1-(2,6-dioxopiperidin-3-yl)-3-methyl-2-oxo-2,3-dihydro-1H-benzo[d]imidazol-5-yl)oxy)acetate. Product was purified with preparative TLC (SiO₂, 60% EtOAc in hexane) to afford tert-butyl 2-((1-(2,6-dioxopiperidin-3-yl)-3-methyl-2-oxo-2,3-dihydro-1H-benzo[d]imidazol-5-yl)oxy)acetate (60 mg, 0.154 mmol, 28%) as off white solid.

LCMS (ESI): 390.1 m/z [M+H]⁺

### Step E

To a solution of tert-butyl 2-((1-(2,6-dioxopiperidin-3-yl)-3-methyl-2-oxo-2,3-dihydro-1H-benzo[d]imidazol-5-yl)oxy)acetate (60 mg, 0.154 mmol) in DCM (2 mL) was added a dropwise TFA (1 mL) at 0° C. and the reaction mixture was allowed to stir at ambient temperature for 16 h under nitrogen. After complete consumption of the starting material (reaction monitored by TLC and LCMS) the volatiles were evaporated under reduced pressure to get the crude compound which was then purified by triturating with diethyl ether and pentane to get 2-((1-(2,6-dioxopiperidin-3-yl)-3-methyl-2-oxo-2,3-dihydro-1H-benzo[d]imidazol-5-yl)oxy)acetic acid (45 mg, 0.135 mmol, 87%) as off white solid.

LCMS (ESI): 334.2 m/z [M+H]⁺

### Step F

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) and 2-((1-(2,6-dioxopiperidin-3-yl)-3-methyl-2-oxo-2,3-dihydro-1H-benzo[d]imidazol-5-yl)oxy)acetic acid (10.9 mg, 0.033 mmol) were dissolved under argon atmosphere in dry DMF (0.989 mL) and DIPEA (0.016 mL, 0.089 mmol), followed by HATU (11.8 mg, 0.031 mmol) was added. Reaction (monitored with LCMS) was stirred for 20 min under argon. After complete consumption of the starting material the mixture was diluted with DCM and washed with brine (2×10 mL) and water (2×10 mL). Organic layer was dried over anhydrous magnesium sulphate, filtered and dried under reduced pressure to give crude tert-butyl 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate as yellow oil, that was directly used in the next step.

LCMS (ESI): 989.0 m/z [M+H]⁺

### Step G

Crude tert-butyl 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate was dissolved in dry DCM (0.500 mL) under argon atmosphere and TFA (0.500 mL) was added. Reaction (monitored with LCMS) was stirred under argon for 16 h. After complete consumption of the starting material the solution was concentrated under reduced pressure and the resulting residue was purified with preparative HPLC (C18, H₂O:MeCN+0.1% FA) to give 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (10.5 mg, 0.011 mmol, 36.7% over 2 steps) as white solid.

LCMS (ESI): 933.15 m/z [M+H]⁺

¹H NMR (500 MHz, DMSO, 353 K) δ 10.77 (s, 1H), 8.24 (dd, J=9.2, 5.9 Hz, 1H), 7.70 (d, J=8.5 Hz, 1H), 7.58 (dd, J=10.4, 2.6 Hz, 1H), 7.45-7.38 (m, 2H), 7.32 (td, J=8.9, 2.6 Hz, 1H), 7.21 (d, J=8.5 Hz, 1H), 6.96 (d, J=8.6 Hz, 1H), 6.87 (dd, J=5.8, 2.8 Hz, 1H), 6.82 (d, J=2.4 Hz, 1H), 6.62 (dd, J=8.6, 2.5 Hz, 1H), 5.25 (dd, J=12.5, 5.5 Hz, 1H), 4.69 (s, 2H), 4.36-4.22 (m, 3H), 4.18 (ddd, J=13.9, 7.8, 5.9 Hz, 1H), 3.75 (s, 3H), 3.40-3.35 (m, 4H), 3.30 (s, 3H), 3.29-3.25 (m, 2H), 2.92-2.83 (m, 1H), 2.73-2.61 (m, 2H), 2.28-2.20 (m, 2H), 2.17-2.01 (m, 7H), 2.01 (s, 3H), 1.88 (s, 3H).

### Example 17: 6-Chloro-1-(2-(4-(2-((1-(2,6-dioxopiperidin-3-yl)-3-methyl-2-oxo-2,3-dihydro-1H-benzo[d]imidazol-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 23)

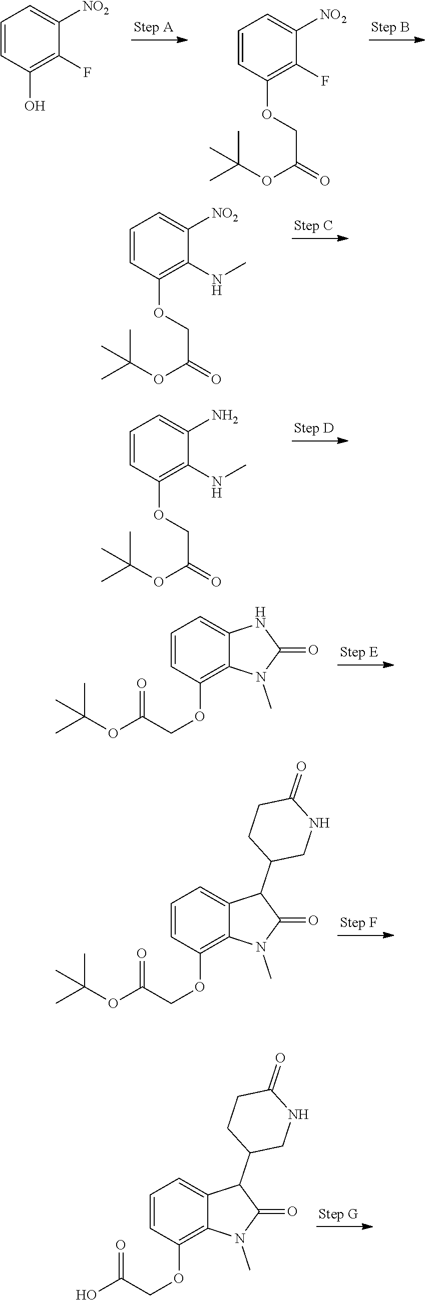

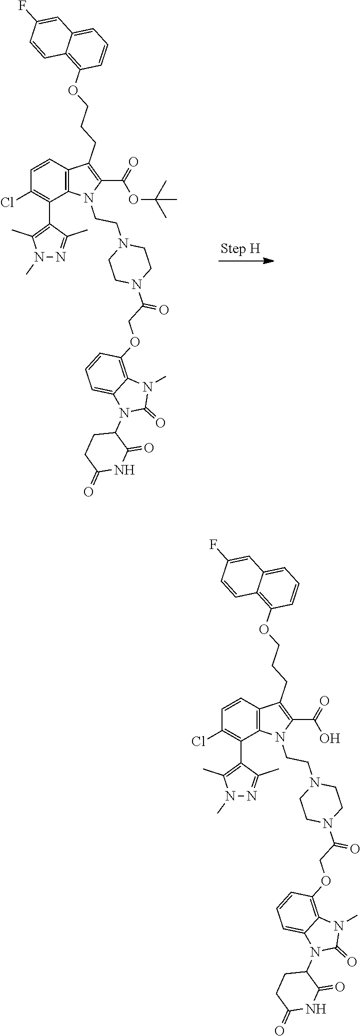

### Step A

2-Fluoro-3-nitrophenol (1 g, 6.365 mmol) was dissolved in DMF (20 mL) and KI (317 mg, 1.91 mmol) and KHCO₃ (764 mg, 7.638 mmol) were added. Then, tert-butyl bromoacetate (1.691 ml, 11.458 mmol) was added dropwise to the reaction mixture and it was then allowed to stir at 60° C. for 5 h under nitrogen. After complete consumption of the starting material the reaction mixture was diluted with ethyl acetate and washed with water and brine. The organic layer was dried over sodium sulphate and evaporated under reduced pressure to get the crude compound, which was then purified by column chromatography (SiO₂, 0-10% EtOAc in hexane) to afford tert-butyl 2-(2-fluoro-3-nitrophenoxy)acetate (1.6 g, 5.90 mmol, 92%) as a yellow solid.

### Step B

To a solution of tert-butyl 2-(2-fluoro-3-nitrophenoxy)acetate (400 mg, 1.476 mmol) in EtOH (8 mL) an aqueous solution of methyl amine (0.33 mL, 7.378 mmol, 40% in H₂O) was added at room temperature and the reaction mixture was allowed to stir for 2 h at 50° C. After complete consumption of the starting material, the volatiles were removed under reduced pressure and crude was purified by column chromatography (SiO₂, 50% EtOAc in hexane) to afford tert-butyl 2-(2-(methylamino)-3-nitrophenoxy)acetate (370 mg, 1.31 mmol, 88%) as brown liquid.

LCMS (ESI): 283.2 m/z [M+H]⁺

### Step C

A solution of tert-butyl 2-(2-(methylamino)-3-nitrophenoxy)acetate (370 mg, 1.312 mmol) in MeOH (10 mL) was degassed with argon for 15 minutes. Then, Pd/C (370 mg) was added slowly under argon. The reaction mixture was then stirred for 4 h at room temperature under H₂ balloon pressure. After complete consumption of the starting material, the reaction mixture was filtered through celite pad and the filtrate was concentrated under reduced pressure. Crude material was purified with column chromatography (SiO₂, 70% EtOAc in hexane) to get tert-butyl 2-(3-amino-2-(methylamino)phenoxy)acetate (225 mg, 0.892 mmol, 68%) of as brown liquid.

LCMS (ESI): 253.0 m/z [M+H]⁺

### Step D

To a solution of tert-butyl 2-(3-amino-2-(methylamino)phenoxy)acetate (225 mg, 0.892 mmol) in THF (12 mL), was added CDI (261 mg, 1.606 mmol) at room temperature under nitrogen atmosphere. The reaction mixture was allowed to stir at 60° C. for 4 h. After the reaction was complete, volatiles were removed under reduced pressure and crude material was purified by column chromatography (SiO₂, 50-60% EtOAc in hexane) to afford tert-butyl 2-((3-methyl-2-oxo-2,3-dihydro-1H-benzo[d]imidazol-4-yl)oxy)acetate (150 mg, 0.539 mmol, 60%) of as a white solid.

LCMS (ESI): 279.2 m/z [M+H]⁺

### Step E

To a well solution of tert-butyl 2-((3-methyl-2-oxo-2,3-dihydro-1H-benzo[d]imidazol-4-yl)oxy)acetate (150 mg, 0.54 mmol) in DMF (4 mL) was added NaH (388 mg, 8.1 mmol) portionwise at 0° C. under nitrogen and the reaction mixture was allowed to stir at the same temperature for 30 minutes. The solution of 3-bromopiperidine-2,6-dione (1.026 g, 5.4 mmol) in DMF (2 mL) was then added dropwise to the reaction mixture at 0° C., which was allowed to stir for 5 min. The mixture was then heated to 80° C. and the reaction was continued for 2 h under nitrogen. After complete consumption of the starting material (monitored by LCMS) the reaction mixture was cooled to −78° C., diluted with EtOAc and stirred for the next 5 min. A saturated solution of ammonium chloride was then added dropwise to the reaction mixture to quench the excess sodium hydride. Organic layer was separated from the solidified aqueous layer and the solid was washed 2 to 3 times with EtOAc. The combined organic layer was washed with brine and evaporated under reduced pressure. Crude compound was purified with preparative TLC (SiO₂, 60% EtOAc in hexane) to afford 60 mg (0.154 mmol, 28%) of tert-butyl 2-((1-(2,6-dioxopiperidin-3-yl)-3-methyl-2-oxo-2,3-dihydro-1H-benzo[d]imidazol-4-yl)oxy)acetate as off white solid.

LCMS (ESI): 390.2 m/z [M+H]⁺

### Step F

To a solution of tert-butyl 2-((1-(2,6-dioxopiperidin-3-yl)-3-methyl-2-oxo-2,3-dihydro-1H-benzo[d]imidazol-4-yl)oxy)acetate (60 mg, 0.154 mmol) in DCM (2 mL) was added TFA (1 mL) at 0° C. dropwise and the reaction mixture was allowed to stir at ambient temperature for 16 h under nitrogen. After complete consumption of the starting material the volatiles were removed under reduced pressure to get the crude compound which was then purified by triturating with diethyl ether and n-pentane to get 2-((1-(2,6-dioxopiperidin-3-yl)-3-methyl-2-oxo-2,3-dihydro-1H-benzo[d]imidazol-4-yl)oxy)acetic acid (30 mg, 0.09 mmol, 58%) of as off white solid.

LCMS (ESI): 334.2 m/z [M+H]⁺

### Step G

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) and 2-((1-(2,6-dioxopiperidin-3-yl)-3-methyl-2-oxo-2,3-dihydro-1H-benzo[d]imidazol-4-yl)oxy)acetic acid (10.9 mg, 0.033 mmol) were dissolved in dry DMF (1.0 mL) under argon atmosphere and DIPEA (0.016 mL, 0.089 mmol), followed by HATU (11.8 mg, 0.031 mmol) as a solution id DMF (1.0 mL) were added. Reaction (monitored with LCMS) was stirred for 15 min at room temperature under argon. After complete conversion of the starting material the solution was diluted with DCM up to 10 mL. The resulting solution was washed with brine (2×10 mL) and water (2×10 mL), dried over anhydrous magnesium sulphate, filtered and dried under reduced pressure. Crude tert-butyl 6-chloro-1-{2-[4-(2-{[1-(2,6-dioxopiperidin-3-yl)-3-methyl-2-oxo-2,3-dihydro-1H-1,3-benzodiazol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (41.8 mg) was obtained as yellow oil.

LCMS (ESI): 989.2 m/z [M+H]⁺

### Step H

Crude tert-butyl 6-chloro-1-{2-[4-(2-{[1-(2,6-dioxopiperidin-3-yl)-3-methyl-2-oxo-2,3-dihydro-1H-1,3-benzodiazol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (41.8 mg) was dissolved in dry DCM (0.500 mL) under argon atmosphere and TFA (0.500 mL, 6.529 mmol) was added. Reaction was stirred under argon for 16 h. After complete consumption of the starting material the solution was concentrated under reduced pressure and the resulting residue purified with preparative HPLC (C18, H₂O:MeCN+0.1% FA) to give 6-chloro-1-(2-(4-(2-((1-(2,6-dioxopiperidin-3-yl)-3-methyl-2-oxo-2,3-dihydro-1H-benzo[d]imidazol-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (13.9 mg, 0.015 mmol, 50% over 2 steps) as white solid.

LCMS (ESI): 933.2 m/z [M+H]⁺

¹H NMR (500 MHz, DMSO, 353 K) δ 10.75 (s, 1H), 8.24 (dd, J=9.3, 5.9 Hz, 1H), 7.70 (d, J=8.5 Hz, 1H), 7.58 (dd, J=10.4, 2.6 Hz, 1H), 7.46-7.38 (m, 2H), 7.32 (td, J=8.9, 2.6 Hz, 1H), 7.21 (d, J=8.5 Hz, 1H), 6.93 (t, J=8.2 Hz, 1H), 6.87 (dd, J=5.6, 3.0 Hz, 1H), 6.78-6.67 (m, 2H), 5.31-5.23 (m, 1H), 4.84 (s, 2H), 4.35-4.12 (m, 4H), 3.75 (s, 3H), 3.57 (s, 3H), 3.41-3.33 (m, 4H), 3.31-3.25 (m, 2H), 2.92-2.85 (m, 1H), 2.74-2.62 (m, 2H), 2.28-2.20 (m, 2H), 2.16-2.02 (m, 7H), 2.01 (s, 3H), 1.88 (s, 3H).

### Example 18: 6-Chloro-1-(2-{4-[({[2-(2,6-dioxopiperidin-3-yl)-1-oxo-2,3-dihydro-1H-isoindol-5-yl]methyl}carbamoyl)methyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (Compound 26)

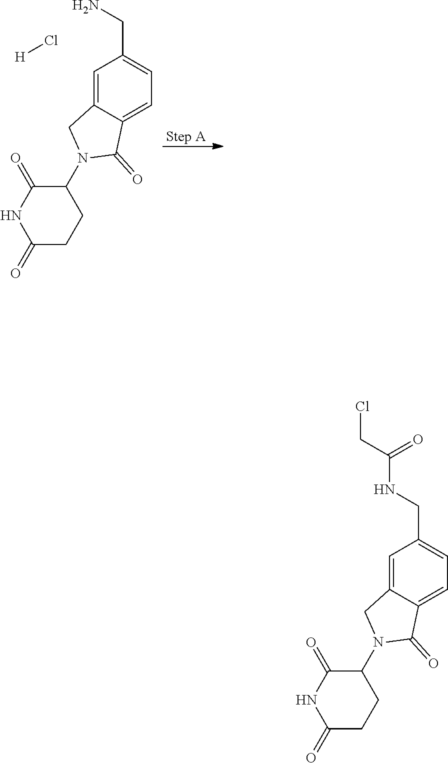 [SMILES: NCc1ccc2c(c1)CN(C1CCC(=O)NC1=O)C2=O ; Cl ; O=C(CCl)NCc1ccc2c(c1)CN(C1CCC(=O)NC1=O)C2=O]

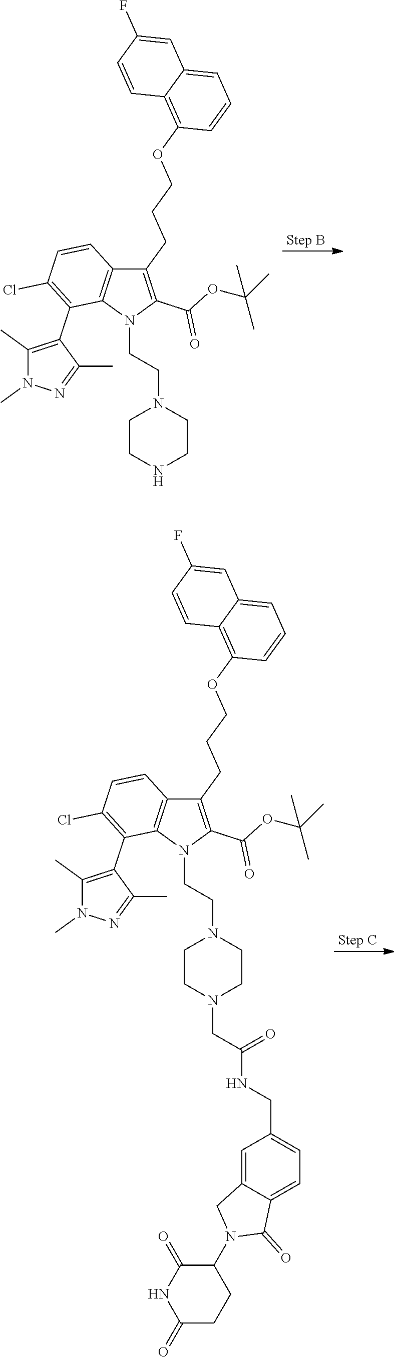

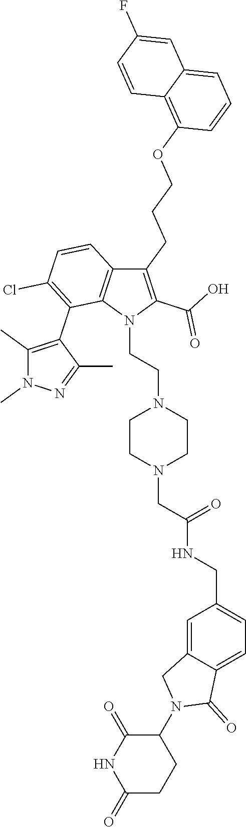 [SMILES: Cc1nn(C)c(C)c1-c1c(Cl)ccc2c(CCCOc3cccc4cc(F)ccc34)c(C(=O)O)n(CCN3CCN(CC(=O)NCc4ccc5c(c4)CN(C4CCC(=O)NC4=O)C5=O)CC3)c12]

### Step A

To the solution of 3-[5-(aminomethyl)-1-oxo-2,3-dihydro-1H-isoindol-2-yl]piperidine-2,6-dione hydrochloride (50.0 mg, 0.161 mmol) in DMF (1.5 mL) was added DIPEA (0.042 mL, 0.242 mmol), followed by chloroacetyl chloride (0.014 mL, 0.178 mmol). Mixture was stirred for overnight at RT. DMF was evaporated and the resultant residue was partitioned between EtOAc and H₂O. The organic layer was further washed with brine, dried over Na₂SO₄, filtered and evaporated. 2-Chloro-N-((2-(2,6-dioxopiperidin-3-yl)-1-oxoisoindolin-5-yl)methyl)acetamide (60.0 mg, 0.161 mmol, 99.8%) was obtained as orangish solid.

LCMS (ESI): 349.9 m/z [M+H]⁺

### Step B

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (35.0 mg, 0.052 mmol) and 2-chloro-N-((2-(2,6-dioxopiperidin-3-yl)-1-oxoisoindolin-5-yl)methyl)acetamide (23.6 mg, 0.067 mmol) were dissolved in DMF (1.0 mL). Subsequently, KI (8.6 mg, 0.052 mmol) and DIPEA (0.027 mL, 0.156 mmol) were added and the mixture was stirred at 70° C. for 24 h. After the reaction was completed, DMF was evaporated and the resulting residue was partitioned between EtOAc and water. The organic layer was further washed with brine, dried over sodium sulphate, filtered and evaporated. Desired product was purified by preparative TLC (SiO₂, 10% MeOH in DCM). tert-Butyl 6-chloro-1-(2-(4-(2-(((2-(2,6-dioxopiperidin-3-yl)-1-oxoisoindolin-5-yl)methyl)amino)-2-oxoethyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (13.0 mg, 0.012 mmol, 22.3%) was obtained as orange oil.

LCMS (ESI): 986.9 m/z [M+H]⁺

### Step C

To a stirred solution of tert-butyl 6-chloro-1-(2-(4-(2-(((2-(2,6-dioxopiperidin-3-yl)-1-oxoisoindolin-5-yl)methyl)amino)-2-oxoethyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (16.0 mg, 0.016 mmol) in DCM (1.0 mL) at 0° C., TFA (1.0 mL) was added dropwise and the resulting reaction mixture was stirred for 1 day at room temperature. After complete consumption of the starting material, the reaction mixture concentrated under reduced pressure. Then, the crude was purified by reverse phase column chromatography (C18, H₂O:MeCN+0.1% FA) to afford 6-chloro-1-(2-{4-[({[2-(2,6-dioxopiperidin-3-yl)-1-oxo-2,3-dihydro-1H-isoindol-5-yl]methyl}carbamoyl)methyl]piperazin-1-yl}ethyl)-3-{3-[(6-fluoronaphthalen-1-yl)oxy]propyl}-7-(1,3,5-trimethyl-1H-pyrazol-4-yl)-1H-indole-2-carboxylic acid (4.9 mg, 0.005 mmol, 32.5%) as a white solid.

LCMS (ESI): 931.2 m/z [M+H]⁺

1H NMR (500 MHz, DMSO) δ=10.97 (s, 1H), 8.30 (t, J=6.3, 1H), 8.24 (dd, J=9.2, 5.9, 1H), 7.72-7.61 (m, 3H), 7.47-7.40 (m, 3H), 7.40-7.32 (m, 2H), 7.18 (d, J=8.5, 1H), 6.87 (dd, J=5.2, 3.5, 1H), 5.10 (dd, J=13.3, 5.1, 1H), 4.46-4.35 (m, 3H), 4.33-4.23 (m, 2H), 4.19 (t, J=6.2, 2H), 4.16-4.09 (m, 1H), 3.70 (d, J=3.7, 3H), 3.22 (t, J=7.6, 2H), 2.97-2.84 (m, 3H), 2.62-2.55 (m, 1H), 2.53-2.51 (m, 1H), 2.44-2.29 (m, 5H), 2.25-2.11 (m, 6H), 2.11-2.04 (m, 1H), 2.03-1.96 (m, 4H), 1.86 (s, 3H).

### Example 19: 6-Chloro-3-[3-(4-chloro-3,5-dimethylphenoxy)propyl]-1-(2-{4-[({[2-(2,6-dioxopiperidin-3-yl)-1-oxo-2,3-dihydro-1H-isoindol-5-yl]methyl}carbamoyl)methyl]piperazin-1-yl}ethyl)-7-(1,3,5-trimethyl-1H-pyrazol-4-yl)-1H-indole-2-carboxylic acid (Compound 27)

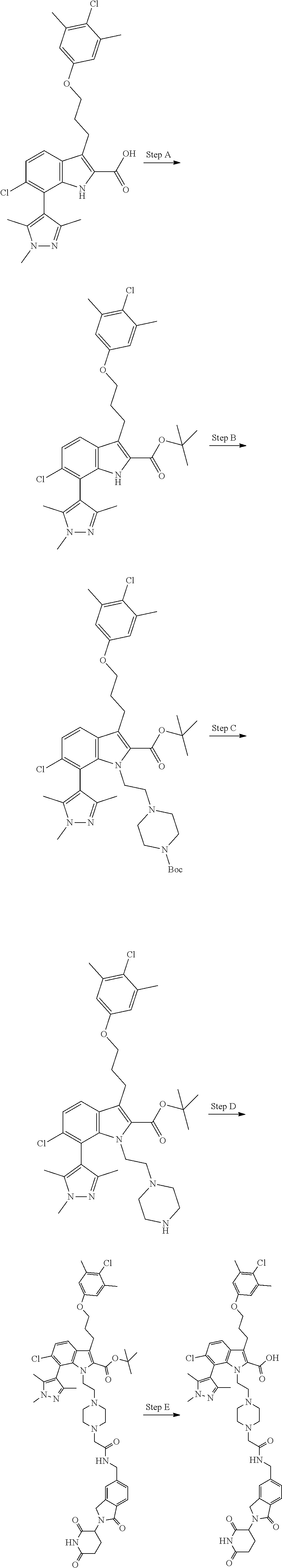

### Step A

6-Chloro-3-(3-(4-chloro-3,5-dimethylphenoxy)propyl)-7-(1,3,5-trimethyl-1H-pyrazol-4-yl)-1H-indole-2-carboxylic acid (3.3 g, 6.613 mmol) was suspended in toluene (30 ml) and the mixture was heated to reflux under nitrogen. N,N-dimethylformamide di-tert-butyl acetal (12.655 ml, 52.906 mmol) was added dropwise to the refluxing mixture. The mixture was heated under reflux for 16 h under nitrogen. After complete consumption of the starting material the reaction mixture was diluted with ethyl acetate, washed successively with sodium bicarbonate (sat), water and brine, organic layer was dried over sodium sulphate and evaporated under reduced pressure to get the crude compound, which was then purified by column chromatography over (SiO₂, 70% ethyl acetate in hexane) to get tert-butyl 6-chloro-3-(3-(4-chloro-3,5-dimethylphenoxy)propyl)-7-(1,3,5-trimethyl-1H-pyrazol-4-yl)-1H-indole-2-carboxylate (2.3 g, 4.136 mmol, 62.49%) as light brown solid.

LCMS (ESI): 556.1 m/z [M+H]⁺

### Step B

To a well stirred solution of tert-butyl 6-chloro-3-(3-(4-chloro-3,5-dimethylphenoxy)propyl)-7-(1,3,5-trimethyl-1H-pyrazol-4-yl)-1H-indole-2-carboxylate (1.6 g, 2.883 mmol) in DMF (20 ml) was added compound-tert-butyl 4-(2-chloroethyl)piperazine-1-carboxylate (1.435 g, 5.766 mmol) followed by cesium carbonate (4.685 g, 14.414 mmol) in DMF (20 ml) and the mixture was allowed to stir at 90° C. for 16 h under nitrogen. After complete consumption of the starting material the reaction mixture was diluted with ethyl acetate, washed successively with water and brine, the organic layer was dried over sodium sulphate and evaporated under reduced pressure to get the crude compound, which was then purified by column chromatography (SiO₂, ethyl acetate in hexane) to get tert-butyl 1-(2-(4-(tert-butoxycarbonyl)piperazin-1-yl)ethyl)-6-chloro-3-(3-(4-chloro-3,5-dimethylphenoxy)propyl)-7-(1,3,5-trimethyl-1H-pyrazol-4-yl)-1H-indole-2-carboxylate (1.6 g, 2.083 mmol, 72.19%) as off white solid.

LCMS (ESI): 768.6 m/z [M+H]⁺

### Step C

tert-Butyl 1-(2-(4-(tert-butoxycarbonyl)piperazin-1-yl)ethyl)-6-chloro-3-(3-(4-chloro-3,5-dimethylphenoxy)propyl)-7-(1,3,5-trimethyl-1H-pyrazol-4-yl)-1H-indole-2-carboxylate (0.5 g, 0.652 mmol) dissolved in 10 ml of 4 M HCl in dioxane at 0° C. and the mixture stirred for 2 h under nitrogen at same temperature. After complete consumption of the starting material the reaction mixture was poured in to cold 1 M NaOH solution and extracted several times with dichloromethane. The combined organics were dried over sodium sulphate and concentrated in vacuo to get the crude compound which was then purified by column chromatography (SiO₂, 10% methanol in dichloromethane) to get tert-Butyl 6-chloro-3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylate (0.2 g, 0.299 mmol, 45.88%) as off white solid.

LCMS (ESI): 668.2 m/z [M+H]⁺

### Step D

tert-Butyl 6-chloro-3-[3-(4-chloro-3,5-dimethylphenoxy)propyl]-1-[2-(piperazin-1-yl)ethyl]-7-(1,3,5-trimethyl-1H-pyrazol-4-yl)-1H-indole-2-carboxylate (35.0 mg, 0.052 mmol) and 2-chloro-N-((2-(2,6-dioxopiperidin-3-yl)-1-oxoisoindolin-5-yl)methyl)acetamide (23.8 mg, 0.068 mmol) were dissolved in DMF (1.0 mL). Subsequently, KI (8.7 mg, 0.052 mmol) and DIPEA (0.027 mL, 0.157 mmol) were added and the mixture was stirred at 70° C. for 24 h. After the reaction was completed, DMF was evaporated and the resulting residue was partitioned between EtOAc and H₂O. The organic layer was further washed with brine, dried over Na₂SO₄, filtered and evaporated. Desired product was purified by preparative TLC (SiO₂, 5% MeOH in DCM). tert-Butyl 6-chloro-3-(3-(4-chloro-3,5-dimethylphenoxy)propyl)-1-(2-(4-(2-(((2-(2,6-dioxopiperidin-3-yl)-1-oxoisoindolin-5-yl)methyl)amino)-2-oxoethyl)piperazin-1-yl)ethyl)-7-(1,3,5-trimethyl-1H-pyrazol-4-yl)-1H-indole-2-carboxylate (16.0 mg, 0.014 mmol, 27.4%) was obtained as orange sticky oil, which was used for the next step without further purification.

LCMS (ESI): 980.99 m/z [M+H]⁺

### Step E

To a solution of tert-butyl 6-chloro-3-[3-(4-chloro-3,5-dimethylphenoxy)propyl]-1-(2-{4-[({[2-(2,6-dioxopiperidin-3-yl)-1-oxo-2,3-dihydro-1H-isoindol-5-yl]methyl}carbamoyl)methyl]piperazin-1-yl}ethyl)-7-(1,3,5-trimethyl-1H-pyrazol-4-yl)-1H-indole-2-carboxylate (16.0 mg, 0.016 mmol) in DCM (0.815 mL) at 0° C., TFA (1.0 mL, 13.059 mmol) was added dropwise and the resulting reaction mixture was stirred for 1 day at room temperature. After complete consumption of the starting material, the reaction mixture was concentrated under reduced pressure. Then, the crude was purified by reverse phase column chromatography (C18, H₂O:MeCN+0.1% FA) to afford the 6-chloro-3-[3-(4-chloro-3,5-dimethylphenoxy)propyl]-1-(2-{4-[({[2-(2,6-dioxopiperidin-3-yl)-1-oxo-2,3-dihydro-1H-isoindol-5-yl]methyl}carbamoyl)methyl]piperazin-1-yl}ethyl)-7-(1,3,5-trimethyl-1H-pyrazol-4-yl)-1H-indole-2-carboxylic acid (6.0 mg, 0.006 mmol, 39.6%) as a white solid.

LCMS (ESI): 925.25 m/z [M+H]⁺

¹H NMR (500 MHz, DMSO) δ=10.97 (s, 1H), 8.30 (t, J=6.3, 1H), 7.70-7.60 (m, 2H), 7.44 (s, 1H), 7.38 (d, J=7.8, 1H), 7.22 (d, J=8.5, 1H), 6.73 (s, 2H), 5.10 (dd, J=13.3, 5.1, 1H), 4.47-4.33 (m, 3H), 4.33-4.22 (m, 2H), 4.18-4.06 (m, 1H), 3.96 (t, J=6.5, 2H), 3.71 (d, J=3.5, 3H), 3.09 (t, J=7.4, 2H), 2.97-2.84 (m, 3H), 2.64-2.55 (m, 1H), 2.54-2.50 (m, 1H), 2.46-2.29 (m, 5H), 2.26 (s, 6H), 2.23-2.05 (m, 5H), 2.05-1.96 (m, 6H), 1.87 (s, 3H).

### Example 20: 6-Chloro-1-{2-[4-(2-{[2-(2,6-dioxopiperidin-3-yl)-1-oxo-2,3-dihydro-1H-isoindol-4-yl]oxy}acetyl)-2,6-dimethylpiperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 28)

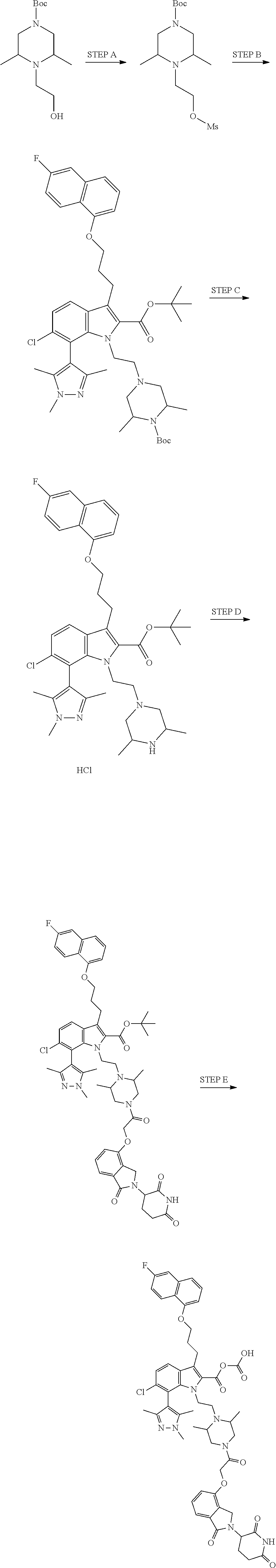

### Step A

tert-Butyl 4-(2-hydroxyethyl)-3,5-dimethylpiperazine-1-carboxylate (52 mg, 0.201 mmol) was dissolved in DCM (4.0 mL), Et₃N (0.084 mL, 0.604 mmol) and DMAP (2.5 mg, 0.020 mmol) were added and reaction mixture cooled to 0° C. Then MsCl (0.031 mL, 0.403 mmol) was added dropwise and reaction mixture was let to stir at RT for 2 h. The crude was washed with brine, dried over MgSO₄, filtered and concentrated in vacuo. The isolated product tert-butyl 4-[2-(methanesulfonyloxy)ethyl]-3,5-dimethylpiperazine-1-carboxylate (45 mg, crude) was orange oil.

### Step B

tert-Butyl 6-chloro-3-{3-[(6-fluoronaphthalen-1-yl)oxy]propyl}-7-(1,3,5-trimethyl-1H-pyrazol-4-yl)-1H-indole-2-carboxylate (25.0 mg, 0.044 mmol), tert-butyl 4-[2-(methanesulfonyloxy)ethyl]-3,5-dimethylpiperazine-1-carboxylate (45 mg, crude) and Cs₂CO₃ (58.0 mg, 0.178 mmol) were dissolved in dry DMF (2.0 mL) in an inert atmosphere and was stirred and stirred at 60° C. for 18 h. After complete consumption of the starting material the reaction was dissolved in DCM and washed with brine. The organic layer was dried over MgSO₄, filtered and concentrated in vacuo to obtain 35.0 mg crude of tert-butyl 1-(2-{4-[(tert-butoxy)carbonyl]-2,6-dimethylpiperazin-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate as a yellow oil.

LCMS (ESI): 802.4 m/z [M+H]⁺

### Step C

To a solution of tert-butyl 1-(2-{4-[(tert-butoxy)carbonyl]-2,6-dimethylpiperazin-1-yl}ethyl)-6-chloro-3-{3-[(6-fluoronaphthalen-1-yl)oxy]propyl}-7-(1,3,5-trimethyl-1H-pyrazol-4-yl)-1H-indole-2-carboxylate (35 mg, crude) in THF (4.0 mL) in 0° C. was added 4 M HCl in dioxane (2.0 mL, 8.000 mmol). The mixture was stirred at RT for 40 h. The solvent was evaporated to obtain tert-butyl 6-chloro-1-[2-(2,6-dimethyl piperazin-1-yl)ethyl]-3-{3-[(6-fluoronaphthalen-1-yl)oxy]propyl}-7-(1,3,5-trimethyl-1H-pyrazol-4-yl)-1H-indole-2-carboxylate hydrochloride (28.9 mg, curde) as a yellow solid which was used to next step without further purification.

LCMS (ESI): 702.4 m/z [M+H]⁺

### Step D

To a solution of tert-butyl 6-chloro-1-[2-(2,6-dimethylpiperazin-1-yl)ethyl]-3-{3-[(6-fluoronaphthalen-1-yl)oxy]propyl}-7-(1,3,5-trimethyl-1H-pyrazol-4-yl)-1H-indole-2-carboxylate hydrochloride (28.9 mg, crude), 2-{[2-(2,6-dioxopiperidin-3-yl)-1-oxo-2,3-dihydro-1H-isoindol-4-yl]oxy}acetic acid (14.9 mg, 0.047 mmol) in dry DMF (1.8 mL) were added HATU (29.7 mg, 0.078 mmol) and DIPEA (0.020 mL, 0.117 mmol). The mixture was stirred at RT for 25 min. The crude was purified by preparative HPLC (C18, H₂O:MeCN+0.1% FA) to obtain tert-butyl 6-chloro-1-{2-[4-(2-{[2-(2,6-dioxopiperidin-3-yl)-1-oxo-2,3-dihydro-1H-isoindol-4-yl]oxy}acetyl)-2,6-dimethylpiperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (8.0 mg, 0.008 mmol, 19% over 3 steps) as a white powder.

LCMS (ESI): 1002.3 m/z [M+H]⁺

### Step E

The solution of tert-butyl 6-chloro-1-{2-[4-(2-{[2-(2,6-dioxopiperidin-3-yl)-1-oxo-2,3-dihydro-1H-isoindol-4-yl]oxy}acetyl)-2,6-dimethylpiperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (8.0 mg, 0.008 mmol) in FA (1.0 mL, 23.400 mmol) was stirred at 55° C. for 14 h. Then the crude was concentrated in vacuo to obtain 6-chloro-1-{2-[4-(2-{[2-(2,6-dioxopiperidin-3-yl)-1-oxo-2,3-dihydro-1H-isoindol-4-yl]oxy}acetyl)-2,6-dimethyl piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (5.5 mg, 0.006 mmol, 72.8%) as a white solid.

LCMS (ESI): 946.2 m/z [M+H]⁺

1H NMR (500 MHz, DMSO, 353 K) δ 10.64 (s, 1H), 8.26 (dd, J=9.2, 5.9 Hz, 1H), 7.71 (d, J=8.5 Hz, 1H), 7.59 (dd, J=10.4, 2.6 Hz, 1H), 7.47-7.40 (m, 3H), 7.37-7.29 (m, 2H), 7.23 (d, J=8.5 Hz, 1H), 7.14 (d, J=8.0 Hz, 1H), 6.88 (dd, J=5.2, 3.4 Hz, 1H), 5.05 (dd, J=13.0, 5.2 Hz, 1H), 4.90 (s, 2H), 4.39 (d, J=17.1 Hz, 1H), 4.34-4.26 (m, 2H), 4.28-4.18 (m, 3H), 3.76 (s, 3H), 3.30-3.23 (m, 2H), 2.88 (ddd, J=17.4, 13.4, 5.5 Hz, 1H), 2.67-2.59 (m, 1H), 2.39-2.34 (m, 1H), 2.29-2.20 (m, 2H), 2.09-1.98 (m, 5H), 1.91 (s, 3H), 1.34-1.25 (m, 4H), 0.69 (d, J=6.3 Hz, 3H), 0.66 (d, J=6.3 Hz, 3H). 3 protons in aliphatic area overlap with DMSO.

### Example 21: 6-Chloro-1-{2-[4-({[2-(2,6-dioxopiperidin-3-yl)-1-oxo-2,3-dihydro-1H-isoindol-4-yl]carbamoyl}methyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (Compound 29)

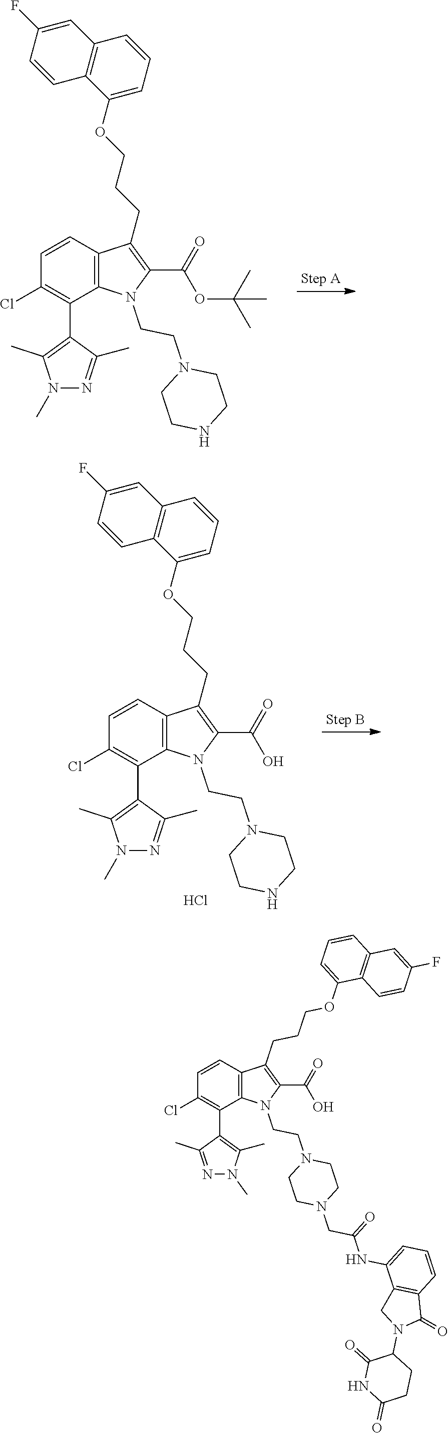 [SMILES: Cc1nn(C)c(C)c1-c1c(Cl)ccc2c(CCCOc3cccc4cc(F)ccc34)c(C(=O)OC(C)(C)C)n(CCN3CCNCC3)c12 ; Cc1nn(C)c(C)c1-c1c(Cl)ccc2c(CCCOc3cccc4cc(F)ccc34)c(C(=O)O)n(CCN3CCNCC3)c12 ; Cl ; Cc1nn(C)c(C)c1-c1c(Cl)ccc2c(CCCOc3cccc4cc(F)ccc34)c(C(=O)O)n(CCN3CCN(CC(=O)Nc4cccc5c4CN(C4CCC(=O)NC4=O)C5=O)CC3)c12]

Step A

To a solution of tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (106.0 mg, 0.157 mmol) in THF (1.0 mL) was added 4M HCl in dioxane (2.1 mL, 61.053 mmol). The mixture was stirred for 18 h at room temperature and next 18 h at 40° C. The crude was concentrated in vacuo and dissolved in 1M HCl to obtain 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylic acid hydrochloride (107.0 mg, 0.163 mmol, quantitative) as a white solid.

LCMS (ESI): 618.3 m/z [M+H]⁺

### Step B

To a stirred solution of 2-chloro-N-[2-(2,6-dioxopiperidin-3-yl)-1-oxo-2,3-dihydro-1H-isoindol-4-yl]acetamide (16.2 mg, 0.048 mmol) and DIPEA (0.024 mL, 0.137 mmol) in DMF (0.600 mL) was added 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylic acid hydrochloride (30.0 mg, 0.046 mmol). The mixture was stirred at 70° C. overnight. Solvents were removed under reduced pressure and the desired product was purified using flash chromatography (SiO₂, DCM: MeOH gradient from 0 to 10% of MeOH), followed by preparative HPLC (H₂O:MeCN+0.1% FA) to give 6-chloro-1-{2-[4-({[2-(2,6-dioxopiperidin-3-yl)-1-oxo-2,3-dihydro-1H-isoindol-4-yl]carbamoyl}methyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (1.3 mg, 0.001 mmol, 3.2%) as a white solid.

LCMS (ESI): 917.2 m/z [M+H]⁺

1H NMR (500 MHz, DMSO, 353 K) 5=10.68 (s, 1H), 9.43 (s, 1H), 8.25 (dd, J=9.3, 5.9, 1H), 7.80 (ddd, J=7.7, 3.5, 1.3, 1H), 7.67 (d, J=8.5, 1H), 7.58 (dd, J=10.3, 2.6, 1H), 7.55-7.45 (m, 2H), 7.45-7.38 (m, 2H), 7.33 (td, J=9.0, 2.6, 1H), 7.19 (d, J=8.5, 1H), 6.87 (dd, J=5.4, 3.3, 1H), 5.07 (ddd, J=13.0, 5.2, 2.4, 1H), 4.40 (d, J=4.0, 2H), 4.33-4.19 (m, 3H), 4.23-4.13 (m, 1H), 3.75 (s, 3H), 3.32-3.21 (m, 2H), 3.11 (s, 2H), 2.93-2.83 (m, 1H), 2.68-2.60 (m, 1H), 2.43-2.34 (m, 1H), 2.28-2.08 (m, 8H), 2.11-2.03 (m, 1H), 2.02 (s, 3H), 1.89 (s, 3H). Note: 4 protons in aliphatic area overlaps with signals from solvents.

### Example 22: 6-Chloro-1-(2-(4-(2-((2-(2,6-dioxopiperidin-3-yl)-1-oxoisoindolin-4-yl)oxy)acetyl)piperazin-1-yl)ethyl)-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylic acid (Compound 36)

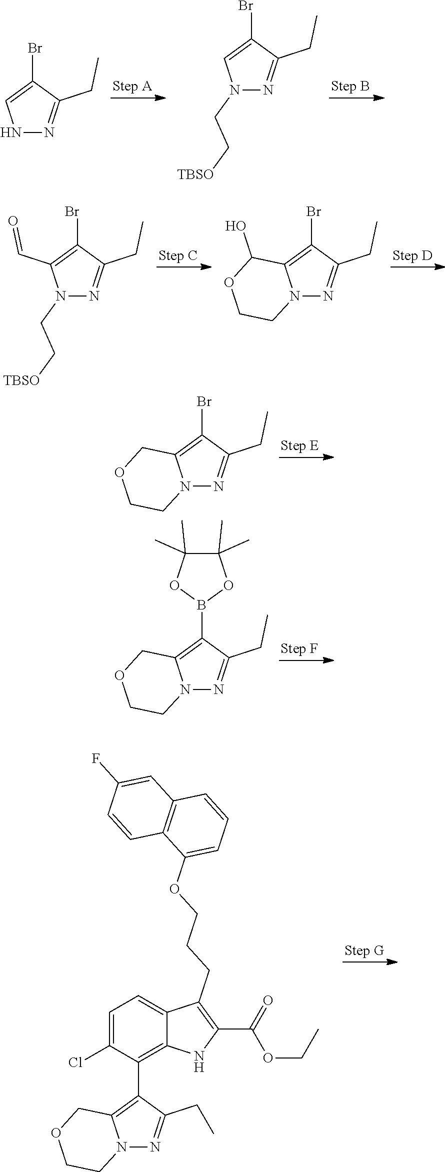

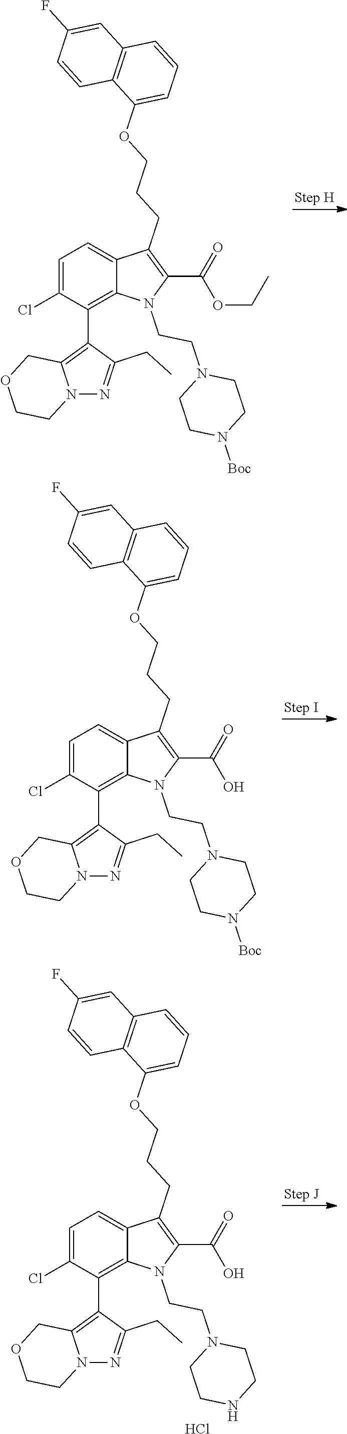 [SMILES: CCOC(=O)c1c(CCCOc2cccc3cc(F)ccc23)c2ccc(Cl)c(-c3c(CC)nn4c3COCC4)c2n1CCN1CCN(C(=O)OC(C)(C)C)CC1 ; CCc1nn2c(c1-c1c(Cl)ccc3c(CCCOc4cccc5cc(F)ccc45)c(C(=O)O)n(CCN4CCN(C(=O)OC(C)(C)C)CC4)c13)COCC2 ; CCc1nn2c(c1-c1c(Cl)ccc3c(CCCOc4cccc5cc(F)ccc45)c(C(=O)O)n(CCN4CCNCC4)c13)COCC2 ; Cl]

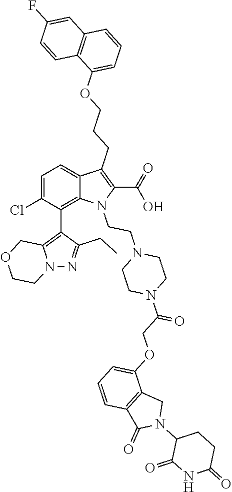 [SMILES: CCc1nn2c(c1-c1c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

### Step A

To a stirred solution of 4-bromo-3-ethyl-1H-pyrazole (6 g, 34.28 mmol) in MeCN (150 mL) was added Cs₂CO₃ (16.711 g, 51.42 mmol), followed by a solution of (2-bromoethoxy)(tert-butyl)dimethylsilane (8.19 ml, 37.70 mmol) dropwise at ambient temperature under nitrogen. The reaction mixture was allowed to stir at room temperature for 16 h. After complete consumption of the starting material (monitored by TLC and LCMS) the reaction mixture was filtered through celite pad and washed with diethyl ether (50 mL). The filtrate was concentrated and the resultant residue was purified by column chromatography (SiO₂, 0-5% EtOAc in n-hexane) to get 4-bromo-1-(2-((tert-butyldimethylsilyl)oxy)ethyl)-3-ethyl-1H-pyrazole (5.5 g, 16.51 mmol) of as colorless oil.

LCMS (ESI): 334.9 m/z [M+H]⁺

### Step B

To a well stirred solution of 2 M LDA (18.61 mL, 36.32 mmol) in THF was added a solution of 4-bromo-1-(2-((tert-butyldimethylsilyl)oxy)ethyl)-3-ethyl-1H-pyrazole (5.5 g, 16.51 mmol) in THF (50 mL) dropwise at −78° C. under nitrogen. The reaction mixture was allowed to stir at same temperature for 30 minutes. To the mixture was added DMF (6.356 mL, 82.55 mmol) dropwise at −78° C. and the resulting reaction mixture was stirred for another 1 h at same temperature. After complete consumption of the starting material (monitored by TLC and LCMS) the excess LDA was quenched by saturated ammonium chloride. The reaction mixture was diluted with ethyl acetate and washed with water and brine. The organic layer was dried over sodium sulphate and evaporated under reduced pressure to get the crude compound, which was then purified by column chromatography (SiO₂, 15-20% EtOAc in n-hexane) to get 4-bromo-1-(2-((tert-butyldimethylsilyl)oxy)ethyl)-3-ethyl-1H-pyrazole-5-carbaldehyde (3 g, 8.31 mmol, 50%) of as white sticky solid.

LCMS (ESI): 362.9 m/z [M+H]⁺

### Step C

To a stirred solution of 4-bromo-1-(2-((tert-butyldimethylsilyl)oxy)ethyl)-3-ethyl-1H-pyrazole-5-carbaldehyde (3 g, 8.31 mmol) in 2-methyl THF (6 mL) and water (6 mL) was added TFA (12 mL) dropwise at RT under nitrogen. The reaction mixture was allowed to stir at RT for 1 h. After complete consumption of the starting material (monitored by TLC and LCMS) the volatiles were evaporated under reduced pressure. Crude mixture was diluted with ethyl acetate and washed with saturated sodium bicarbonate solution, water and brine. The organic layer was dried over sodium sulphate and evaporated under reduced pressure to get the crude 3-bromo-2-ethyl-6,7-dihydro-4H-pyrazolo[5,1-c][1,4]oxazin-4-ol (1.8 g) as white solid which was then directly used for next step without further purification.

LCMS (ESI): 249.0 m/z [M+H]⁺

### Step D

To a well stirred solution of crude 3-bromo-2-ethyl-6,7-dihydro-4H-pyrazolo[5,1-c][1,4]oxazin-4-ol (1.8 g, 7.28 mmol) in DCM (35 mL) was added TFA (4.98 mL, 43.725 mmol), and triethyl silane (2.53 mL, 21.86 mmol) at 0° C. under nitrogen. The reaction mixture was allowed to stir at 0° C. for 1 h. After that again trifluoroacetic acid (4.98 mL, 43.72 mmol), and triethyl silane (2.53 mL, 21.86 mmol) were added successively and the reaction mixture was allowed to stir for 16 h at RT. After complete consumption of starting material (monitored by TLC and LCMS) the volatiles of the reaction mixture were evaporated under reduced pressure. Crude mass was diluted with DCM and washed with saturated bicarbonate solution and brine. The organic layer was dried over sodium sulphate and evaporated under reduced pressure to get the crude compound, which was then purified by column chromatography (SiO₂, 30-40% EtOAc in n-hexane) to get 3-bromo-2-ethyl-6,7-dihydro-4H-pyrazolo[5,1-c][1,4]oxazine (1.5 g, 6.64 mmol, 80% over two steps) of as off white powder.

LCMS (ESI): 232.7 m/z [M+H]⁺

### Step E

To a well stirred solution of 3-bromo-2-ethyl-6,7-dihydro-4H-pyrazolo[5,1-c][1,4]oxazine (1.534 g, 6.64 mmol) and 2-Isopropoxy-4,4,5,5-tetramethyl-1,3,2-dioxaborolane (6.17 mL, 33.2 mmol) in THF (30 mL), was added n-butyllithium (2 M solution in THF, 7.3 mL, 14.60 mmol) at −78° C. under argon. The resulting mixture was then allowed to stir at −78° C. for 2 h. The mixture was slowly warmed to RT and was stirred for another 30 minutes. After complete consumption of the starting material (monitored by TLC and LCMS) the excess butyllithium was quenched by addition of saturated ammonium chloride. The solution was diluted with ethyl acetate and washed with water and brine. The organic layer was dried over sodium sulphate and evaporated under reduced pressure to get the crude compound, which was then purified by column chromatography (SiO₂, 10-20% EtOAc in n-hexane) to get 2-ethyl-3-(4,4,5,5-tetramethyl-1,3,2-dioxaborolan-2-yl)-6,7-dihydro-4H-pyrazolo[5,1-c][1,4]oxazine (270 mg, 0.96 mmol, 15%) of as white solid.

LCMS (ESI): 279.0 m/z [M+H]⁺

### Step F

To a stirred solution of ethyl 7-bromo-6-chloro-3-(3-((6-fluoronaphthalen-1-yl)oxy)propyl)-1H-indole-2-carboxylate (270 mg, 0.53 mmol) in dioxane (2.5 mL) and water (0.25 mL) were added 2-ethyl-3-(4,4,5,5-tetramethyl-1,3,2-dioxaborolan-2-yl)-6,7-dihydro-4H-pyrazolo[5,1-c][1,4]oxazine (270 mg, 0.96 mmol) and K₃PO₄ (337.5 mg, 1.59 mmol) at room temperature under nitrogen. The mixture was deoxygenated with argon and to it was added Pd(dtbpf)Cl₂ (69.29 mg, 0.106 mmol) under argon atmosphere. Then the reaction mixture was heated under reflux for 16 h. After complete consumption of the starting material (monitored by TLC and LCMS) the reaction mixture was filtered through celite pad and the filtrate was evaporated under reduced pressure to get the residue. It was then diluted with ethyl acetate and washed with water and brine. The organic layer was dried over sodium sulphate and evaporated under reduced pressure to get the crude ethyl 6-chloro-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylate (270 mg) as a brown sticky solid which was then directly used for next step without further purification.

LCMS (ESI): 576.0 m/z [M+H]⁺

### Step G

To a well stirred solution of ethyl 6-chloro-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylate (270 mg, 0.469 mmol) in DMF (3 mL) was added tert-butyl 4-(2 chloroethyl)piperazine-1-carboxylate (174.37 mg, 0.703 mmol) followed by cesium carbonate (229.092 mg, 0.703 mmol) at RT under nitrogen. The resulting mixture was allowed to stir at 90° C. for 16 h under nitrogen. After complete consumption of the starting material (monitored by TLC and LCMS) the reaction mixture was diluted with ethyl acetate and washed with water and brine. The organic layer was dried over sodium sulphate and evaporated under reduced pressure to get crude compound which was then purified by column chromatography (SiO₂, 100% EtOAc) to get ethyl 1-(2-(4-(tert-butoxycarbonyl)piperazin-1-yl)ethyl)-6-chloro-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylate (160 mg, 0.20 mmol, 38% over two steps) as a brown sticky solid.

LCMS (ESI): 788.4 m/z [M+H]⁺

### Step H

Ethyl 1-(2-(4-(tert-butoxycarbonyl)piperazin-1-yl)ethyl)-6-chloro-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylate (160 mg. 0.20 mmol) was dissolved in EtOH (2 mL) and a solution of NaOH (32.529 mg, 0.813 mmol) in water (0.5 mL) was added to it. The mixture was heated under reflux for 16 h. After complete consumption of the starting material (monitored by TLC and LCMS) the reaction mixture was cooled down to room temperature and solvents were evaporated under reduced pressure. The residue was diluted with water and washed with ethyl acetate. Aqueous layer was carefully acidified using 1 M HCl to pH=~3, extracted with ethyl acetate (3×50 mL), dried over Na₂SO₄, filtered, and concentrated in vacuo to afford crude 1-(2-(4-(tert-butoxycarbonyl)piperazin-1-yl)ethyl)-6-chloro-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylic acid (135 mg) as light brown gummy solid which was directly used for next step without further purification.

LCMS (ESI): 760.4 m/z [M+H]⁺

### Step I

Crude 1-(2-(4-(tert-butoxycarbonyl)piperazin-1-yl)ethyl)-6-chloro-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylic acid (135 mg) was dissolved in 4M HCl in Dioxane (12 mL) at 0° C. and the mixture was stirred for 2 h under nitrogen at room temperature. Where upon LCMS indicated the reaction was complete, the volatiles were concentrated under vacuum to get the crude compound which was then purified by triturating with diethyl ether and n-pentane to afford 6-chloro-7-(2-ethyl-6,7-dihydro-4H-pyrazolo[5,1-c][1,4]oxazin-3-yl)-3-(3-((6-fluoronaphthalen-1-yl)oxy)propyl)-1-(2-(piperazin-1-yl)ethyl)-1H-indole-2-carboxylic acid hydrochloride (55 mg, 0.08 mmol, 41% over two steps) of as off white solid.

LCMS (ESI): 660.4 m/z [M+H]⁺

### Step J

2-{[2-(2,6-Dioxopiperidin-3-yl)-1-oxo-2,3-dihydro-1H-isoindol-4-yl]oxy}acetic acid (12.3 mg, 0.039 mmol) and HATU (13.1 mg, 0.034 mmol) were dissolved in dry DMF (2.7 mL) under argon atmosphere and DIPEA (0.011 mL, 0.064 mmol) was added. Reaction was stirred for 1 h at room temperature. 6-Chloro-7-{2-ethyl-4H,6H,7H-pyrazolo[3,2-c][1,4]oxazin-3-yl}-3-{3-[(6-fluoronaphthalen-1-yl)oxy]propyl}-1-[2-(piperazin-1-yl)ethyl]-1H-indole-2-carboxylic acid hydrochloride (15.0 mg, 0.022 mmol) and DIPEA (0.011 mL, 0.064 mmol) were dissolved in DMSO in a separate vial and added dropwise to the reaction mixture. Reaction (monitored with LCMS) was stirred for the next 20 min. After complete consumption of the starting material reaction mixture was passed through a syringe filter and purified directly with reverse-phase preparative HPLC (H₂O/MeCN with 0.1% FA) to give 6-chloro-1-(2-(4-(2-((2-(2,6-dioxopiperidin-3-yl)-1-oxoisoindolin-4-yl)oxy)acetyl)piperazin-1-yl)ethyl)-7-(2-ethyl-6,7-dihydro-4H-pyrazolo[5,1-c][1,4]oxazin-3-yl)-3-(3-((6-fluoronaphthalen-1-yl)oxy)propyl)-1H-indole-2-carboxylic acid (7.1 mg, 0.007 mmol, 34.4%) as white solid.

LCMS (ESI): 960.1 m/z [M+H]⁺

¹H NMR (500 MHz, DMSO, 353 K) δ 10.64 (s, 1H), 8.22 (dd, J=9.2, 5.9 Hz, 1H), 7.70 (d, J=8.6 Hz, 1H), 7.58 (dd, J=10.4, 2.6 Hz, 1H), 7.50-7.36 (m, 3H), 7.36-7.26 (m, 2H), 7.20 (d, J=8.6 Hz, 1H), 7.14 (d, J=8.1 Hz, 1H), 6.86 (dd, J=5.9, 2.7 Hz, 1H), 5.04 (dd, J=13.0, 5.2 Hz, 1H), 4.88 (s, 2H), 4.56 (d, J=15.0 Hz, 1H), 4.52 (d, J=14.9 Hz, 1H), 4.45-4.36 (m, 2H), 4.31 (d, J=17.1 Hz, 1H), 4.23 (t, J=6.2 Hz, 2H), 4.21-4.14 (m, 1H), 4.14-4.04 (m, 4H), 3.39-3.34 (m, 4H), 3.29-3.25 (m, 2H), 2.87 (ddd, J=17.3, 13.4, 5.5 Hz, 1H), 2.66-2.59 (m, 1H), 2.48-2.40 (m, 1H), 2.37-2.28 (m, 1H), 2.26-2.20 (m, 3H), 2.19-2.08 (m, 6H), 2.07-2.01 (m, 1H), 1.00 (t, J=7.5 Hz, 3H).

### Example 23: 6-Chloro-1-(2-(4-((4-(2-(2,6-dioxopiperidin-3-yl)-3-methyl-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 145)

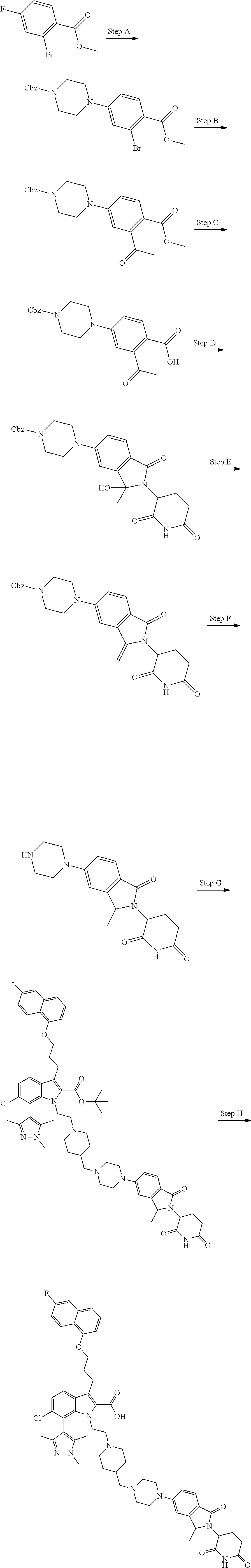

### Step A

To a solution of methyl 2-bromo-4-fluorobenzoate (800 mg, 3.43 mmol) in DMSO (5 mL) were added DIPEA (1.1 mL, 6.93 mmol) and benzyl piperazine-1-carboxylate (1.34 mL, 6.93 mmol). The resulting solution was stirred for 16 h at 110° C. The reaction mixture was then diluted with ethyl acetate and washed with cold water and brine. The organic layer was dried over Na₂SO₄ and evaporated to get crude compound. Benzyl 4-(3-bromo-4-(methoxycarbonyl)phenyl)piperazine-1-carboxylate (1.1 g, 2.53 mmol, 74%) was purified by column chromatography (SiO₂, 40-50% EtOAc in DCM).

LCMS (ESI): 435.0 m/z [M+H]⁺.

### Step B

To a solution of benzyl 4-(3-bromo-4-(methoxycarbonyl)phenyl)piperazine-1-carboxylate (500 mg, 1.16 mmol) in toluene (5 mL) was added tributyl(1-ethoxyvinyl)stannane (0.19 mL, 0.56 mmol) at room temperature and the mixture was bubbled with argon for 15 min. Tetrakis(triphenylphosphine) palladium(0) (26.75 mg, 0.023 mmol) was added and the reaction mixture was stirred at 110° C. overnight. After complete consumption of starting materials (monitored by TLC and LCMS) the reaction mixture was filtered through a celite bed and the filtrate was evaporated to get the crude intermediate which was immediately dissolved in THF (6 mL) and 1M HCl (3 mL) and stirred for 3 h at room temperature. After completion (monitored by TLC and LCMS) the volatiles were evaporated, the residue taken up in water and extracted with ethyl acetate. The organic layer was washed by brine, dried over Na₂SO₄ and evaporated. Benzyl 4-(3-acetyl-4-(methoxycarbonyl)phenyl)piperazine-1-carboxylate (300 mg, 0.75 mmol, 65%) was purified by column chromatography (SiO₂, 10-20% EtOAc in hexane).

LCMS (ESI): 397.2 m/z [M+H]⁺.

### Step C

To a stirred solution of benzyl 4-(3-acetyl-4-(methoxycarbonyl)phenyl)piperazine-1-carboxylate (300 mg, 0.75 mmol) in THF (6 mL) and water (3 mL) was added NaOH (241.6 mg, 6.04 mmol) at 0° C. and the reaction mixture was stirred for 3 h. After complete consumption of the starting material (monitored by TLC and LCMS) the volatiles were evaporated, the resulting residue was dissolved in water and acidified with 1M HCl at 0° C. to pH 5-6. The mixture was extracted with ethyl acetate. The combined organic phases were washed with brine and dried over Na₂SO₄ to get crude 2-acetyl-4-(4-((benzyloxy)carbonyl)piperazin-1-yl)benzoic acid (200 mg) as white solid which was directly used in the next step without further purification.

LCMS (ESI): 383.1 m/z [M+H]⁺.

### Step D

To a solution of crude 2-acetyl-4-(4-((benzyloxy)carbonyl)piperazin-1-yl)benzoic acid (200 mg) in DMF (3 mL) was added 3-aminopiperidine-2,6-dione hydrochloride (171.8 g, 1.044 mmol) and DIPEA (0.177 mL, 1.044 mmol) at room temperature under nitrogen. The reaction mixture was stirred for 15 min under the same conditions. HATU (296.75 mg, 0.78 mmol) was added and the resulting mixture was stirred for 1 h at room temperature. After complete consumption of starting materials (monitored by TLC and LCMS) the reaction mixture was quenched with ice-water and volatiles were evaporated. Crude reaction mass was diluted with DCM, washed with saturated NaHCO₃ solution, water and brine, dried over anhydrous Na₂SO₄ and filtered. The filtrate was concentrated to get crude benzyl 4-(2-(2,6-dioxopiperidin-3-yl)-3-hydroxy-3-methyl-1-oxoisoindolin-5-yl)piperazine-1-carboxylate (200 mg) as yellow sticky solid which was directly used in the next step.

LCMS (ESI): m/z not found.

### Step E

To a solution of crude benzyl 4-(2-(2,6-dioxopiperidin-3-yl)-3-hydroxy-3-methyl-1-oxoisoindolin-5-yl)piperazine-1-carboxylate (200 mg) in DCM (2 mL) were added TFA (0.186 mL, 2.436 mmol) and triethylsilane (0.036 mL, 1.22 mmol) at 0° C. under nitrogen. The reaction mixture was stirred at 0° C. for 1 h. Additional portions of TFA (0.19 mL, 2.44 mmol) and triethylsilane (0.036 mL, 1.22 mmol) were added. After complete consumption of starting material (monitored by TLC and LCMS) the reaction mixture was evaporated, the residue was taken up in DCM, washed with saturated NaHCO₃ solution and brine. The organic layer was dried over Na₂SO₄ and filtered. The filtrate was evaporated to get the crude compound which was purified by silica gel chromatography (SiO₂, 3% to 5% MeOH in DCM) to get 100 mg (0.21 mmol, 28% over three steps) of benzyl 4-(2-(2,6-dioxopiperidin-3-yl)-3-methylene-1-oxoisoindolin-5-yl)piperazine-1-carboxylate as a yellow solid.

LCMS (ESI): 475.1 m/z [M+H]⁺.

### Step F

A solution of benzyl 4-(2-(2,6-dioxopiperidin-3-yl)-3-methylene-1-oxoisoindolin-5-yl)piperazine-1-carboxylate (100 mg, 0.21 mmol) in acetic acid (3 mL) was purged with argon for 10 min at room temperature and then Pd/C (100 mg, 10% w/w) was added. The reaction mixture was stirred for 5 h under hydrogen atmosphere (balloon pressure) at room temperature. After consumption of starting material (monitored by LCMS and TLC) the reaction mixture was filtered through a pad of celite and the filtrate was evaporated to get 60 mg (0.175 mmol, 83%) of 3-(3-methyl-1-oxo-5-(piperazin-1-yl)isoindolin-2-yl)piperidine-2,6-dione as a brownish yellow solid.

LCMS (ESI): 343.2 m/z [M+H]⁺.

### Step G

To a solution of tert-butyl 6-chloro-3-(3-((6-fluoronaphthalen-1-yl)oxy)propyl)-1-(2-(4-formylpiperidin-1-yl)ethyl)-7-(1,3,5-trimethyl-1H-pyrazol-4-yl)-1H-indole-2-carboxylate (100 mg, 0.14 mmol) in DCM (5 mL) and methanol (1 mL) were added acetic acid (0.008 mL, 0.14 mmol) followed by 3-(3-methyl-1-oxo-5-(piperazin-1-yl)isoindolin-2-yl)piperidine-2,6-dione (58.14 mg, 0.17 mmol) and the reaction mixture was stirred for 4 h at room temperature. After that time, sodium triacetoxyborohydride (149.81 mg, 0.71 mmol) was added. The reaction mixture was stirred at 80° C. overnight. After complete consumption of the starting materials (monitored by TLC and LCMS) the reaction mixture was filtered through celite pad and the solvents were evaporated to get the crude which was then purified by preparative TLC (SiO₂, 10% MeOH in DCM) to get 70 mg of tert-butyl 6-chloro-1-(2-(4-((4-(2-(2,6-dioxopiperidin-3-yl)-3-methyl-1-oxoisoindolin-5-yl)piperazin-1-yl)methyl) piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate as off white solid with some amount of unreacted tert-butyl 6-chloro-3-(3-((6-fluoronaphthalen-1-yl)oxy)propyl)-1-(2-(4-formylpiperidin-1-yl)ethyl)-7-(1,3,5-trimethyl-1H-pyrazol-4-yl)-1H-indole-2-carboxylate. This mixture was forwarded for the next step.

tert-Butyl 6-chloro-3-(3-((6-fluoronaphthalen-1-yl)oxy)propyl)-1-(2-(4-formylpiperidin-1-yl)ethyl)-7-(1,3,5-trimethyl-1H-pyrazol-4-yl)-1H-indole-2-carboxylate was prepared as described in WO2022/253713A1.

LCMS (ESI): 1027.7 m/z [M+H]⁺.

### Step H

To a crude solution of tert-butyl 6-chloro-1-(2-(4-((4-(2-(2,6-dioxopiperidin-3-yl)-3-methyl-1-oxoisoindolin-5-yl)piperazin-1-yl)methyl) piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (70 mg) in DCM (2 mL) was added TFA (1 mL) dropwise at 0° C. under nitrogen and the reaction mixture was stirred at room temperature for 3 h. After complete consumption of the starting material (monitored by TLC and LCMS) the volatiles were evaporated. The crude compound was purified by reversed phase preparative HPLC (C18, 10 mM NH₄OAc in H₂O:MeCN) to afford 12 mg (0.012 mmol, 8% over two steps) of 6-chloro-1-(2-(4-((4-(2-(2,6-dioxopiperidin-3-yl)-3-methyl-1-oxoisoindolin-5-yl)piperazin-1-yl)methyl) piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid as white solid as mixture of two stereoisomers.

LCMS (ESI): 971.9 m/z [M+H]⁺.

¹H NMR (500 MHz, DMSO, 353 K) δ 10.56-10.47 (m, 1H), 8.26 (dd, J=9.2, 5.8 Hz, 1H), 7.64 (d, J=8.5 Hz, 1H), 7.58 (dd, J=10.4, 2.7 Hz, 1H), 7.47-7.38 (m, 3H), 7.32 (td, J=8.9, 2.7 Hz, 1H), 7.18 (d, J=8.5 Hz, 1H), 6.99 (s, 1H), 7.00-6.94 (m, 1H), 6.87 (dd, J=5.3, 3.4 Hz, 1H), 4.71-4.51 (m, 2H), 4.31-4.21 (m, 3H), 4.21-4.14 (m, 1H), 3.76 (s, 3H), 3.28-3.19 (m, 6H), 2.82-2.67 (m, 1H), 2.62-2.54 (m, 2H), 2.48-2.43 (m, 4H), 2.34-2.20 (m, 5H), 2.19-2.13 (m, 2H), 2.11 (d, J=7.1 Hz, 2H), 2.06-1.94 (m, 4H), 1.91 (s, 1H), 1.89 (s, 3H), 1.66-1.56 (m, 2H), 1.56-1.47 (m, 1H), 1.43 (dd, J=13.4, 6.6 Hz, 3H), 1.20-1.09 (m, 2H).

### Example 24: 6-Chloro-1-(2-(4-((4-(2-(2,6-dioxopiperidin-3-yl)-6-fluoro-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 144)

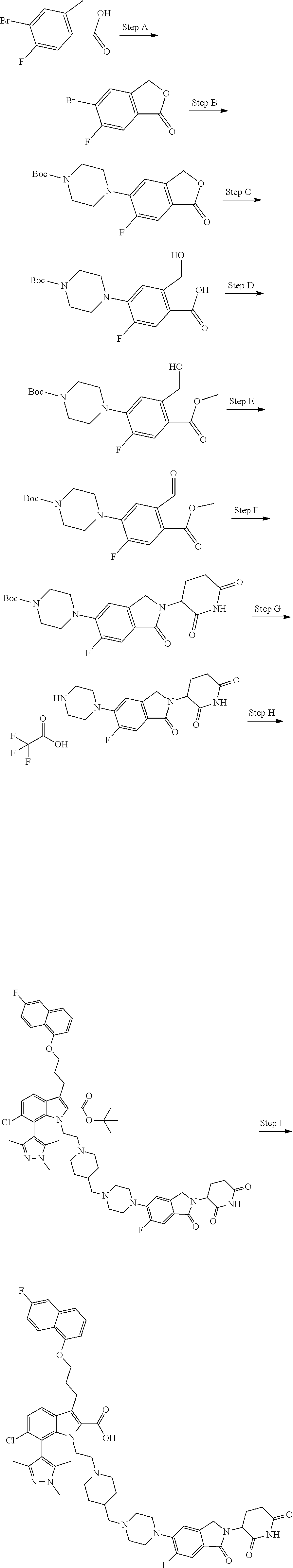

### Step A

To a solution of 4-bromo-5-fluoro-2-methylbenzoic acid (2.5 g, 10.73 mmol) in a mixture of EtOAc:H₂O (110 mL, 6/5 v/v) was added NaBrO₃ (4.88 g, 32.33 mmol), followed by NaHSO₃ (3.36 g, 32.33 mmol) at 0° C. The reaction was stirred at room temperature for 48 h. After completion, the reaction was extracted with ethyl acetate, the combined organic phases were washed with water and brine, dried over anhydrous Na₂SO₄ and concentrated. Crude product was purified by column chromatography (SiO₂, 5-10% EtOAc in hexane) to give 1.5 g (6.49 mmol, 60%) of 5-bromo-6-fluoroisobenzofuran-1(3H)-one as white solid.

LCMS (ESI): 233.2 m/z [M+H]⁺.

### Step B

To a solution of 5-bromo-6-fluoroisobenzofuran-1(3H)-one (1.5 g, 6.49 mmol) in dioxane (40 mL) was added tert-butyl piperazine-1-carboxylate (2.42 g, 12.92 mmol), followed by Cs₂CO₃ (6.33 g, 19.48 mmol) and Xantphos (375 mg, 0.65 mmol). The mixture was purged with argon and Pd₂dba₃ (297 mg, 0.33 mmol) was added under argon. Then the reaction mixture was stirred at 100° C. for 16 h. After complete consumption of the starting materials (monitored by TLC and LCMS), the solvent was evaporated. The resulting residue was diluted with ethyl acetate and washed with water and brine. The combined organic phases were dried over Na₂SO₄ and evaporated to get crude compound which was then purified by column chromatography (SiO₂, 20-30% EtOAc in hexane) to give 700 mg (2.08 mmol, 32%) of tert-butyl 4-(6-fluoro-1-oxo-1,3-dihydroisobenzofuran-5-yl)piperazine-1-carboxylate as off white solid.

LCMS (ESI): 337.1 m/z [M+H]⁺.

### Step C

To a solution of tert-butyl 4-(6-fluoro-1-oxo-1,3-dihydroisobenzofuran-5-yl)piperazine-1-carboxylate (700 mg, 2.08 mmol) in THF (4 mL), methanol (4 mL) and water (4 mL) was added NaOH (333 mg, 8.33 mmol) and the reaction was stirred for 1 h at room temperature. After complete consumption of the starting material (monitored by TLC and LCMS), the reaction mixture was acidified with 1M HCl and extracted with ethyl acetate. The organic phase was dried over Na₂SO₄ and evaporated to give crude 4-(4-(tert-butoxycarbonyl)piperazin-1-yl)-5-fluoro-2-(hydroxymethyl) benzoic acid (600 mg) as off white solid which was used for next step without further purification.

LCMS (ESI): 355.4 m/z [M+H]⁺.

### Step D

To a solution of crude 4-(4-(tert-butoxycarbonyl)piperazin-1-yl)-5-fluoro-2-(hydroxymethyl) benzoic acid (600 mg) in methanol (6 mL) and ethyl acetate (6 mL) was added trimethylsilyldiazomethane (2M in hexanes, 5.3 mL, 10.59 mmol) under argon atmosphere at −10° C. Then the reaction mixture was stirred for 30 min under argon atmosphere at the same temperature. After complete consumption of the starting material (monitored by TLC and LCMS), the solution was quenched with water and extracted with ethyl acetate. The organic phase was dried over Na₂SO₄ and concentrated under reduced pressure to afford crude tert-butyl 4-(2-fluoro-5-(hydroxymethyl)-4-(methoxycarbonyl)phenyl)piperazine-1-carboxylate (500 mg) which was used in the next step without further purification.

LCMS (ESI): 369.2 m/z [M+H]⁺.

### Step E

To a solution of crude tert-butyl 4-(2-fluoro-5-(hydroxymethyl)-4-(methoxycarbonyl) phenyl)piperazine-1-carboxylate (500 mg) in DCM (12 mL) was added Dess-Martin periodinane (1.72 g) at 0° C. under nitrogen. The reaction mixture was stirred at room temperature for 2 h. After complete consumption of the starting material (monitored by TLC), the reaction mixture was quenched with cold water and extracted with DCM. The combined organic layer was dried over Na₂SO₄ and evaporated to get crude product. tert-Butyl 4-(2-fluoro-5-formyl-4-(methoxycarbonyl)phenyl)piperazine-1-carboxylate (500 mg, 1.37 mmol, 65% over three steps) was purified by column chromatography (SiO₂, 20% EtOAc in hexane).

### Step F

To a solution of tert-butyl 4-(2-fluoro-5-formyl-4-(methoxycarbonyl)phenyl)piperazine-1-carboxylate (500 mg, 1.37 mmol) in DCE (10 mL) were added 3-aminopiperidine-2,6-dione hydrochloride (450 mg, 2.74 mmol), acetic acid (1.1 mL 19.13 mmol) and DIPEA (0.6 mL, 3.42 mmol) at room temperature under nitrogen. The reaction mixture was stirred at 80° C. for 4 h and cooled to room temperature. Sodium triacetoxyborohydride (868 mg, 2.73 mmol) was added under nitrogen and the reaction was refluxed for 12 h. After complete consumption of the starting material (monitored by TLC and LCMS), the volatiles were evaporated, and the residue was diluted with ethyl acetate and washed with water and brine. The organic phase was dried over Na₂SO₄ and concentrated to get the crude compound, which was then purified by column chromatography (SiO₂, 5-10% MeOH in DCM) affording 210 mg (0.47 mmol, 34%) of tert-butyl 4-(2-(2,6-dioxopiperidin-3-yl)-6-fluoro-1-oxoisoindolin-5-yl)piperazine-1-carboxylate as off white solid.

LCMS (ESI): 447.3 m/z [M+H]⁺.

### Step G

To a solution of tert-butyl 4-(2-(2,6-dioxopiperidin-3-yl)-6-fluoro-1-oxoisoindolin-5-yl)piperazine-1-carboxylate (210 mg, 0.47 mmol) in DCM (5 mL) was added TFA (3 mL) at 0° C. dropwise. The reaction was stirred at room temperature for 16 h under nitrogen. After complete consumption of the starting material (monitored by TLC and LCMS), the volatiles were evaporated and the residue was triturated with diethyl ether and n-pentane to get 150 mg (0.33 mmol, 69%) of 3-(6-fluoro-1-oxo-5-(piperazin-1-yl)isoindolin-2-yl)piperidine-2,6-dione trifluoroacetate as off white solid.

LCMS (ESI): 347.0 m/z [M+H]⁺.

### Step H

To a solution of tert-butyl 6-chloro-3-(3-((6-fluoronaphthalen-1-yl)oxy)propyl)-1-(2-(4-formylpiperidin-1-yl)ethyl)-7-(1,3,5-trimethyl-1H-pyrazol-4-yl)-1H-indole-2-carboxylate (100 mg, 0.14 mmol) in DCM/methanol (10 mL, 4:1 v/v) was added 3-(6-fluoro-1-oxo-5-(piperazin-1-yl)isoindolin-2-yl)piperidine-2,6-dione trifluoroacetate (99 mg, 0.22 mmol) at room temperature under nitrogen. Acetic acid (8 μL, 0.14 mmol) was added to the reaction mixture at 0° C. followed by sodium triacetoxyborohydride (151 mg, 0.71 mmol). The reaction was stirred at room temperature for 16 h under nitrogen. After complete consumption of the starting materials (monitored by TLC and LCMS), the solvents were evaporated to get the crude material. The residue was taken up in ethyl acetate and washed with water and brine. The organic phase was dried over Na₂SO₄ and concentrated. The residue was triturated with diethyl ether and n-pentane to afford 80 mg of crude tert-butyl 6-chloro-1-(2-(4-((4-(2-(2,6-dioxopiperidin-3-yl)-6-fluoro-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate as off white solid which was directly used for next step without purification.

tert-Butyl 6-chloro-3-(3-((6-fluoronaphthalen-1-yl)oxy)propyl)-1-(2-(4-formylpiperidin-1-yl)ethyl)-7-(1,3,5-trimethyl-1H-pyrazol-4-yl)-1H-indole-2-carboxylate was prepared as described in WO2022/253713A1.

LCMS (ESI): 1031.7 m/z [M+H]⁺.

### Step I

To a solution of crude tert-butyl 6-chloro-1-(2-(4-((4-(2-(2,6-dioxopiperidin-3-yl)-6-fluoro-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (80 mg, 0.077 mmol) in DCM (2 mL) was added TFA (1 mL) dropwise at 0° C. under nitrogen and the reaction mixture was stirred at room temperature for 3 h. After complete consumption of the starting material the volatiles were evaporated. The residue was purified with reversed phase preparative HPLC (C18, H₂O:MeCN+0.1% FA) to afford 18 mg of 6-chloro-1-(2-(4-((4-(2-(2,6-dioxopiperidin-3-yl)-6-fluoro-1-oxoisoindolin-5-yl)piperazin-1-yl)methyl) piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (0.018 mmol, 13.1% over two steps) as white solid.

LCMS (ESI): 976.7 m/z [M+H]⁺

¹H NMR (500 MHz, DMSO) δ 10.95 (s, 1H), 8.25 (dd, J=9.3, 5.9 Hz, 1H), 7.69-7.60 (m, 2H), 7.46-7.33 (m, 4H), 7.20 (d, J=8.5 Hz, 1H), 7.16 (d, J=7.5 Hz, 1H), 6.89-6.83 (m, 1H), 5.06 (dd, J=13.3, 5.1 Hz, 1H), 4.35 (d, J=17.1 Hz, 1H), 4.30-4.05 (m, 5H), 3.75 (s, 3H), 3.20-3.15 (m, 2H), 3.06-2.97 (m, 4H), 2.90 (ddd, J=17.3, 13.7, 5.4 Hz, 1H), 2.63-2.56 (m, 1H), 2.43-2.31 (m, 8H), 2.24-2.14 (m, 2H), 2.06-1.93 (m, 7H), 1.88 (s, 3H), 1.67-1.56 (m, 2H), 1.55-1.47 (m, 1H), 1.33-1.23 (m, 2H), 1.18-1.05 (m, 2H).

### Example 25: 6-Chloro-1-(2-(4-(2-(4-((2,6-dioxopiperidin-3-yl)carbamoyl)-3-ethylphenoxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 25)

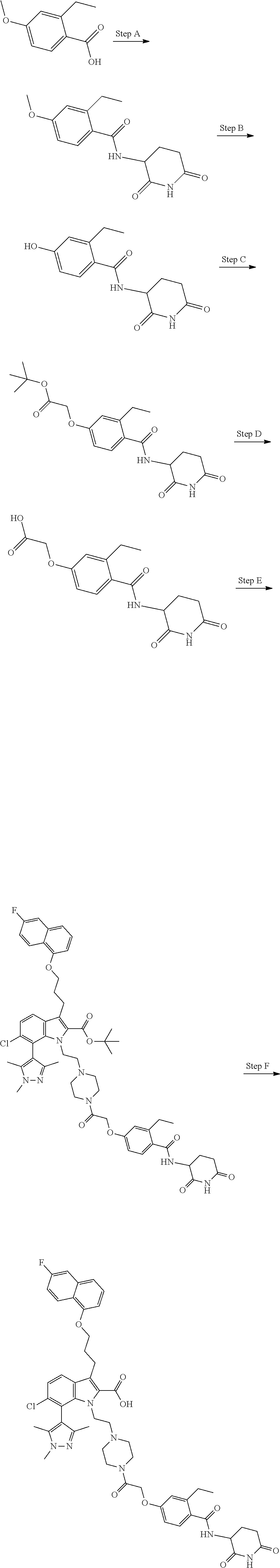

### Step A

To a stirred solution of 2-ethyl-4-methoxybenzoic acid (750 mg, 4.17 mmol) in DMF (12 mL) were added HATU (1.90 g, 5.0 mmol) and DIPEA (1.46 mL, 8.33 mmol) at room temperature under nitrogen. The reaction mixture was stirred for 15 min and 3-aminopiperidine-2,6-dione hydrochloride (1.03 g, 6.24 mmol) was added. The reaction was stirred for 1 h at room temperature. After full conversion (monitored by LCMS), the reaction was quenched by ice-water. The mixture was concentrated under vacuum, diluted with DCM and washed with saturated NaHCO₃ solution, water and brine. The organic phase was dried over anhydrous Na₂SO₄ and filtered. The filtrate was concentrated and crude product was purified by column chromatography (SiO₂, 50-60% EtOAc in DCM) to get 500 mg (1.72 mmol, 41%) of N-(2,6-dioxopiperidin-3-yl)-2-ethyl-4-methoxybenzamide as white solid.

LCMS (ESI): 291.1 m/z [M+H]⁺.

### Step B

To a solution of N-(2,6-dioxopiperidin-3-yl)-2-ethyl-4-methoxybenzamide (500 mg, 1.72 mmol) in DCM (12.0 mL) was added 1M solution of BBr₃ in DCM (8.6 mL, 8.6 mmol) at 0° C. The reaction mixture was stirred for 3 h at 0° C. and allowed to slowly reach room temperature. After consumption of the starting material the mixture was concentrated under reduced pressure and the resulting residue was purified with flash chromatography (SiO₂, 3% MeOH in DCM) to get N-(2,6-dioxopiperidin-3-yl)-2-ethyl-4-hydroxybenzamide (400 mg, 1.45 mmol, 84%) as a white solid.

LCMS (ESI): 277.1 m/z [M+H]⁺.

### Step C

To a solution of N-(2,6-dioxopiperidin-3-yl)-2-ethyl-4-hydroxybenzamide (400 mg, 1.45 mmol) in DMF (5 mL) were added KHCO₃ (435 mg, 4.34 mmol), tert-butyl bromoacetate (0.21 mL, 1.45 mmol) and KI (72.17 mg, 0.44 mmol) at room temperature under nitrogen. The reaction mixture was stirred at 60° C. for 1 h.

After complete consumption of the starting material (monitored by TLC and LCMS), the reaction mixture was diluted with ethyl acetate and washed with water and brine. The organic phase was dried over Na₂SO₄ and concentrated under reduced pressure. The crude was purified with reverse phase preparative HPLC (C18, 10 mM NH₄Cl in H₂O:MeCN) to get 110 mg (0.282 mmol, 19.4%) of tert-butyl 2-(4-((2,6-dioxopiperidin-3-yl)carbamoyl)-3-ethylphenoxy)acetate as white solid.

LCMS (ESI): 391.1 m/z [M+H]⁺.

### Step D

To a solution of tert-butyl 2-(4-((2,6-dioxopiperidin-3-yl)carbamoyl)-3-ethylphenoxy)acetate (100 mg, 0.26 mmol) in DCM (3 mL) was added TFA (1 mL) dropwise at 0° C. The reaction mixture was stirred at room temperature for 16 h under nitrogen. After full conversion (monitored by TLC and LCMS), the solution was concentrated under reduced pressure. The resulting crude product was triturated with diethyl ether and n-pentane to get 65 mg (0.19 mmol, 75%) of 2-(4-((2,6-dioxopiperidin-3-yl)carbamoyl)-3-ethylphenoxy)acetic acid as off white solid.

LCMS (ESI): 335.2 m/z [M+H]⁺.

### Step E

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.03 mmol) and 2-(4-((2,6-dioxopiperidin-3-yl)carbamoyl)-3-ethylphenoxy)acetic acid (11.9 mg, 0.036 mmol) were dissolved in dry DMF (1 mL) and Et₃N (0.016 mL, 0.09 mmol) was added, followed by HATU (12.4 mg, 0.03 mmol) as a solution in DMF (0.5 mL). The reaction was stirred for 15 min at room temperature. After complete consumption of the starting material the solution was diluted with DCM and washed with brine and water. Organic phase was dried over anhydrous MgSO₄, filtered and concentrated under reduced pressure. Crude tert-butyl 6-chloro-1-(2-(4-(2-(4-((2,6-dioxopiperidin-3-yl)carbamoyl)-3-ethylphenoxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (22.5 mg) was obtained as yellow oil and used in the next step without further purification.

LCMS (ESI): 989.8 m/z [M+H]⁺.

### Step F

Crude tert-butyl 6-chloro-1-(2-(4-(2-(4-((2,6-dioxopiperidin-3-yl)carbamoyl)-3-ethylphenoxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (22.5 mg) was dissolved in dry DCM (0.5 mL) under argon atmosphere and TFA (0.5 mL) was added. The reaction was stirred at room temperature under argon for 16 h. After complete consumption of the starting material the solution was concentrated under reduced pressure. The resulting residue was dissolved in DMSO and purified by reversed phase HPLC (C18, H₂O:MeCN+0.1% FA) to give 6-chloro-1-(2-(4-(2-(4-((2,6-dioxopiperidin-3-yl)carbamoyl)-3-ethylphenoxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (9.4 mg, 0.010 mmol, 47% over two steps) as yellow solid.

LCMS (ESI): 934.3 m/z [M+H]⁺.

¹H NMR (500 MHz, DMSO, 353 K) δ 10.49 (s, 1H), 8.24 (dd, J=9.3, 5.9 Hz, 1H), 8.17 (d, J=8.2 Hz, 1H), 7.70 (d, J=8.5 Hz, 1H), 7.59 (dd, J=10.4, 2.7 Hz, 1H), 7.45-7.39 (m, 2H), 7.35-7.30 (m, 2H), 7.21 (d, J=8.5 Hz, 1H), 6.87 (dd, J=5.9, 2.7 Hz, 1H), 6.81 (d, J=2.6 Hz, 1H), 6.76 (dd, J=8.4, 2.6 Hz, 1H), 4.74 (s, 2H), 4.71-4.61 (m, 1H), 4.32-4.22 (m, 3H), 4.18 (ddd, J=13.9, 7.8, 5.9 Hz, 1H), 3.76 (s, 3H), 3.38-3.34 (m, 4H), 3.29-3.26 (m, 2H), 2.80-2.71 (m, 3H), 2.60-2.53 (m, 1H), 2.27-2.20 (m, 2H), 2.16-2.02 (m, 8H), 2.01 (s, 3H), 1.88 (s, 3H), 1.16 (t, J=7.5 Hz, 3H).

### Example 26: 6-Chloro-1-(2-(4-(5-(2,6-dioxopiperidin-3-yl)-1-methyl-1H-indole-2-carbonyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 148)

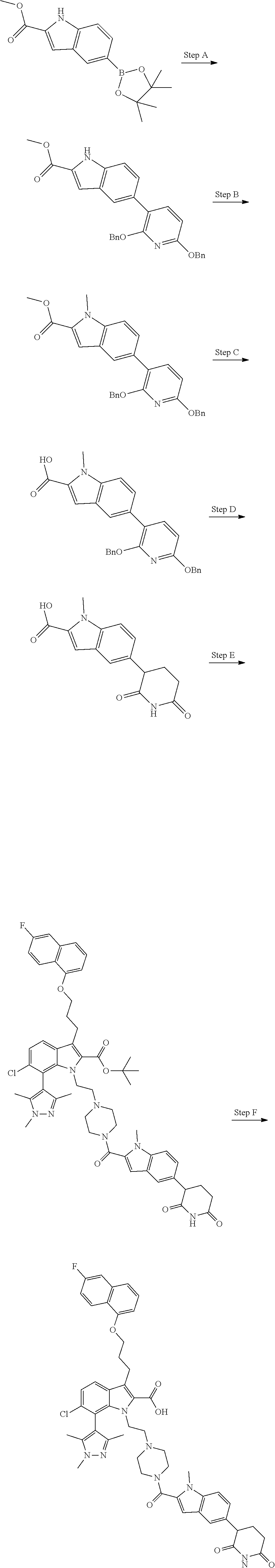

### Step A

To a stirred of solution of methyl 5-(4,4,5,5-tetramethyl-1,3,2-dioxaborolan-2-yl)-1H-indole-2-carboxylate (1 g, 3.32 mmol) and 2,6-bis(benzyloxy)-3-bromopyridine (1.23 g, 3.32 mmol) in dioxane/H₂O (5 mL, 10/1 v/v) in a sealed tube was added K₂CO₃ (1.6 g, 11.62 mmol). The reaction mixture was degassed for 10 min and then PdCl₂(dppf) (120 mg, 0.17 mmol) was added. The reaction mixture was stirred at 100° C. for 16 h. After completion, the mixture was filtered and concentrated under reduced pressure to get crude product which was purified by combi-flash column chromatography to afford 1 g (2.15 mmol, 65%) of methyl 5-(2,6-bis(benzyloxy)pyridin-3-yl)-1H-indole-2-carboxylate.

LCMS (ESI): 465.1 m/z [M+H]⁺.

### Step B

To a solution of methyl 5-(2,6-bis(benzyloxy)pyridin-3-yl)-1H-indole-2-carboxylate (600 mg, 1.29 mmol) in DMF (10 mL) was added NaH (47 mg, 1.94 mmol, 60% suspension in mineral oil) at 0° C. The reaction mixture was stirred for 10 min at 0° C. and iodomethane (0.24 mL, 3.87 mmol) was added. The reaction was carried out at room temperature for 16 h. After completion, the reaction mixture was diluted with cold water and extracted with ethyl acetate. The combined organic layer was washed with brine, dried over Na₂SO₄ and evaporated. Methyl 5-(2,6-bis(benzyloxy)pyridin-3-yl)-1-methyl-1H-indole-2-carboxylate (560 mg, 1.17 mmol, 91%) was purified by flash column chromatography (SiO₂, EtOAc-hexane).

LCMS (ESI): 479.0 m/z [M+H]⁺.

### Step C

To a solution of methyl 5-(2,6-bis(benzyloxy)pyridin-3-yl)-1-methyl-1H-indole-2-carboxylate (560 mg, 1.17 mmol) in THF/MeOH/H₂O (15 mL, 1/1/1 v/v/v) was added LiOH monohydrate (120 mg, 2.93 mmol). The reaction mixture was stirred at room temperature for 16 h. After completion, the solvent was evaporated, the residue was acidified with aqueous NaHSO₄ and extracted with 10% MeOH in DCM. The combined organic layer was washed with brine, dried over Na₂SO₄ and evaporated. Crude 5-(2,6-bis(benzyloxy)pyridin-3-yl)-1-methyl-1H-indole-2-carboxylic acid (500 mg) was forwarded for the next step without further purification.

LCMS (ESI): 463.2 m/z [M−H]⁻.

### Step D

To a solution of crude 5-(2,6-bis(benzyloxy)pyridin-3-yl)-1-methyl-1H-indole-2-carboxylic acid (300 mg, 0.65 mmol) in acetic acid (35 mL) was added Pd/C (300 mg, 10% w/w). The reaction mixture was stirred at room temperature under hydrogen (balloon) for 6 h. After completion, the mixture was filtered through celite bed and filtrate was evaporated. 5-(2,6-Dioxopiperidin-3-yl)-1-methyl-1H-indole-2-carboxylic acid (85 mg, 0.3 mmol, 42% over two steps) was purified by preparative HPLC.

LCMS (ESI): 287.2 m/z [M+H]⁺.

### Step E

Tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20 mg, 0.03 mmol) and 5-(2,6-dioxopiperidin-3-yl)-1-methyl-1H-indole-2-carboxylic acid (10.2 mg, 0.04 mmol) were dissolved in dry DMF (1 mL) under argon atmosphere. DIPEA (0.016 mL, 0.09 mmol) was added, followed by HATU (11.8 mg, 0.03 mmol) as a solution in DMF (0.6 mL). The reaction was stirred at room temperature for 15 min. After complete consumption of the starting material the mixture was diluted with DCM and washed with brine and water. The organic phase was dried over anhydrous MgSO₄, filtered and concentrated under reduced pressure. Crude tert-butyl 6-chloro-1-(2-(4-(5-(2,6-dioxopiperidin-3-yl)-1-methyl-1H-indole-2-carbonyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (35.1 mg) was obtained as yellow oil and used in the subsequent step without further purification.

LCMS (ESI): 942.4 m/z [M+H]⁺.

Step F Crude tert-butyl 6-chloro-1-(2-(4-(5-(2,6-dioxopiperidin-3-yl)-1-methyl-1H-indole-2-carbonyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (35.1 mg) was dissolved in dry DCM (0.5 mL) under argon and TFA (0.5 mL, 6.53 mmol) was added. The reaction was stirred under argon at room temperature for 16 h. After complete consumption of the starting material, the reaction mixture was concentrated under reduced pressure and purified with reversed phase preparative HPLC (C18, H₂O:MeCN+0.1% FA) to give 6-chloro-1-(2-(4-(5-(2,6-dioxopiperidin-3-yl)-1-methyl-1H-indole-2-carbonyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (12.6 mg, 0.014 mmol, 46% over two steps) as a white solid.

LCMS (ESI): 886.2 m/z [M+H]⁺.

¹H NMR (500 MHz, DMSO, 353K) δ 10.47 (s, 1H), 8.23 (dd, J=9.2, 5.9 Hz, 1H), 7.70 (d, J=8.5 Hz, 1H), 7.58 (dd, J=10.4, 2.6 Hz, 1H), 7.46-7.37 (m, 4H), 7.32 (ddd, J=9.2, 8.6, 2.6 Hz, 1H), 7.21 (d, J=8.5 Hz, 1H), 7.12 (dd, J=8.6, 1.8 Hz, 1H), 6.87 (dd, J=6.2, 2.5 Hz, 1H), 6.52 (d, J=0.8 Hz, 1H), 4.38-4.26 (m, 1H), 4.24 (t, J=6.3 Hz, 2H), 4.22-4.16 (m, 1H), 3.91 (dd, J=10.2, 5.1 Hz, 1H), 3.75 (s, 3H), 3.71 (s, 3H), 3.56-3.44 (m, 4H), 3.31-3.25 (m, 2H), 2.71-2.61 (m, 1H), 2.55-2.51 (m, 1H), 2.28-2.20 (m, 3H), 2.20-2.06 (m, 7H), 2.01 (s, 3H), 1.89 (s, 3H).

### Example 27: 1-(2-(2-Carboxy-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazin-1-ium trifluoroacetate (Compound 147)

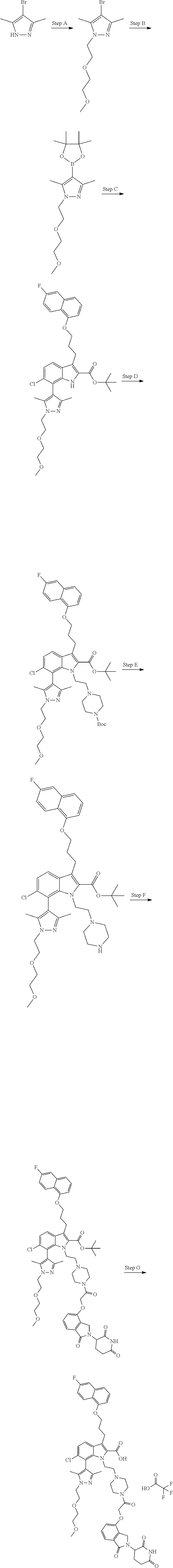

### Step A

To a solution of 4-bromo-3,5-dimethyl-1H-pyrazole (5 g, 28.57 mmol) in DMF (50 mL) was added 1-bromo-2-(2-methoxyethoxy)ethane (5.07 ml, 37.14 mmol) followed by Cs₂CO₃ (18.57 g, 57.14 mmol) at room temperature under nitrogen. The reaction mixture was stirred at room temperature for 2 h. After complete consumption of the starting materials the reaction mixture was diluted with ethyl acetate and washed with water and brine. The organic layer was dried over Na₂SO₄ and concentrated under reduced pressure. The residue was triturated with diethyl ether and n-pentane to get 4.8 g (17.33 mmol, 60%) of 4-bromo-1-(2-(2-methoxyethoxy)ethyl)-3,5-dimethyl-1H-pyrazole as off white solid.

LCMS (ESI): 276.8 m/z [M+H]⁺.

### Step B

To a solution of 4-bromo-1-(2-(2-methoxyethoxy)ethyl)-3,5-dimethyl-1H-pyrazole (1 g, 3.61 mmol) and 2-isopropoxy-4,4,5,5-tetramethyl-1,3,2-dioxaborolane (3.70 ml, 18.12 mmol) in THF (20 mL) was added n-butyllithium (2M in hexanes, 4.5 mL, 9.058 mmol) at −78° C. under nitrogen. The reaction mixture was stirred at the same temperature for 2 h. After full conversion the excess of n-butyllithium was quenched by saturated NH₄Cl solution at −78° C. and the reaction was extracted with ethyl acetate. The combined organic fractions were washed with brine, dried over Na₂SO₄ and concentrated under reduced pressure. Crude 1-(2-(2-methoxyethoxy)ethyl)-3,5-dimethyl-4-(4,4,5,5-tetramethyl-1,3,2-dioxaborolan-2-yl)-1H-pyrazole (1 g) was used in the next step without further purification.

LCMS (ESI): 325.0 m/z [M+H]⁺.

### Step C

To a solution of tert-butyl 7-bromo-6-chloro-3-(3-((6-fluoronaphthalen-1-yl)oxy)propyl)-1H-indole-2-carboxylate (300 mg, 0.57 mmol) in dioxane (4 mL) and water (1 mL) were added crude 1-(2-(2-methoxyethoxy)ethyl)-3,5-dimethyl-4-(4,4,5,5-tetramethyl-1,3,2-dioxaborolan-2-yl)-1H-pyrazole (734 mg) and Cs₂CO₃ (552 mg, 1.70 mmol). The mixture was deoxygenated with argon and PdCl₂(dtbpf) (36 mg, 0.056 mmol) was added under nitrogen atmosphere. The reaction mixture was stirred at 100° C. for 1 h until complete consumption of the starting materials. The reaction mixture was diluted with ethyl acetate and washed successively with water and brine. The organic layer was dried over Na₂SO₄ and concentrated under reduced pressure. tert-Butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (240 mg, 0.37 mmol, 65%) was purified by column chromatography (SiO₂, 20% EtOAc in hexane).

LCMS (ESI): 650.4 m/z [M+H]⁺.

### Step D

To a solution of tert-butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (240 mg, 0.37 mmol) in DMF (5 mL) were added tert-butyl 4-(2-chloroethyl)piperazine-1-carboxylate (275 mg, 1.11 mmol) and Cs₂CO₃ (360 mg, 1.11 mmol) at room temperature. The reaction mixture was stirred at 90° C. for 16 h under nitrogen. After complete consumption of the starting materials the reaction mixture was diluted with ethyl acetate and washed with water and brine. The organic layer was dried over Na₂SO₄ and concentrated under reduced pressure. The crude was purified by column chromatography (SiO₂, 30% EtOAc in hexane) to afford 240 mg (0.28 mmol, 75%) of tert-butyl 1-(2-(4-(tert-butoxycarbonyl)piperazin-1-yl)ethyl)-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate as white solid.

LCMS (ESI): 862.8 m/z [M+H]⁺.

### Step E

To a solution of tert-butyl 1-(2-(4-(tert-butoxycarbonyl)piperazin-1-yl)ethyl)-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (240 mg, 0.28 mmol) in dioxane (3 mL) was added 6 mL of 4M HCl in dioxane at 0° C. under nitrogen. The reaction mixture was stirred with cooling to 0° C. for 2 h under nitrogen. After complete consumption of the starting material (monitored by TLC and LCMS), the reaction mixture was poured into cold 1M NaOH solution and extracted with dichloromethane. The combined organic fractions were dried over Na₂SO₄ and concentrated in vacuo. Crude compound was purified by trituration with diethyl ether and n-pentane to get 200 mg (0.26 mmol, 94%) of tert-butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1-(2-(piperazin-1-yl)ethyl)-1H-indole-2-carboxylate as white solid.

LCMS (ESI): 762.6 m/z [M+H]⁺.

### Step F

To a solution of tert-butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1-(2-(piperazin-1-yl)ethyl)-1H-indole-2-carboxylate (180 mg, 0.24 mmol) in DMF (5 mL) were added 2-((2-(2,6-dioxopiperidin-3-yl)-1-oxoisoindolin-4-yl)oxy)acetic acid (75 mg, 0.24 mmol), HATU (134 mg, 0.36 mmol) and DIPEA (0.082 mL, 0.47 mmol) at room temperature under nitrogen. The mixture was stirred at room temperature for 1 h under nitrogen. After completion of the reaction, the mixture was poured into ice-cold water and extracted with ethyl acetate. The organic layer was washed with brine, dried over anhydrous Na₂SO₄ and concentrated under reduced pressure. The crude compound was purified by column chromatography (SiO₂, 10% MeOH in DCM) to afford 100 mg (0.094 mmol, 39%) of tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate as off white solid.

LCMS (ESI): 1062.4 m/z [M+H]⁺.

### Step G

To a solution of tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (100 mg, 0.094 mmol) in DCM (5 mL) was added TFA (1 mL) dropwise at 0° C. under nitrogen. The mixture was stirred at room temperature for 16 h under nitrogen. After complete consumption of the starting material (monitored by TLC and LCMS), the volatiles were evaporated. Crude compound was purified by trituration with diethyl ether and n-pentane to get 93 mg (0.083 mmol, 83%) of 1-(2-(2-carboxy-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazin-1-ium trifluoroacetate as white solid.

LCMS (ESI): 1006.6 m/z [M+H]⁺.

¹H NMR (500 MHz, DMSO, 353 K) δ 10.63 (s, 1H), 8.24 (dd, J=9.2, 5.8 Hz, 1H), 7.72 (d, J=8.6 Hz, 1H), 7.58 (dd, J=10.4, 2.6 Hz, 1H), 7.46-7.38 (m, 3H), 7.35-7.29 (m, 2H), 7.22 (d, J=8.5 Hz, 1H), 7.13 (d, J=8.1 Hz, 1H), 6.87 (dd, J=5.9, 2.8 Hz, 1H), 5.04 (dd, J=13.0, 5.1 Hz, 1H), 4.88 (s, 2H), 4.39 (d, J=17.1 Hz, 1H), 4.31 (d, J=17.1 Hz, 1H), 4.28-4.15 (m, 6H), 3.85-3.76 (m, 2H), 3.55 (dd, J=5.8, 4.1 Hz, 2H), 3.45 (dd, J=5.9, 4.0 Hz, 2H), 3.38-3.33 (m, 4H), 3.31-3.27 (m, 2H), 3.26 (s, 3H), 2.93-2.82 (m, 1H), 2.63-2.57 (m, 1H), 2.46-2.40 (m, 1H), 2.28-2.21 (m, 2H), 2.17-2.03 (m, 7H), 2.01 (s, 3H), 1.92 (s, 3H).

### Example 28: 6-Chloro-1-(2-(4-((3-(2,4-dioxotetrahydropyrimidin-1(2H)-yl)-2-methylquinolin-8-yl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (Compound 146)

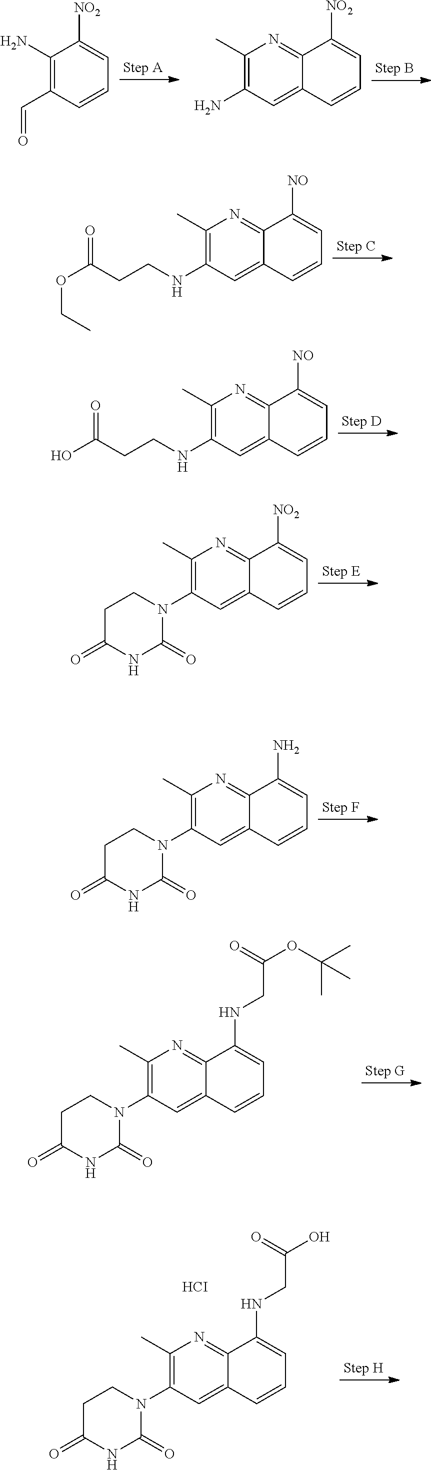

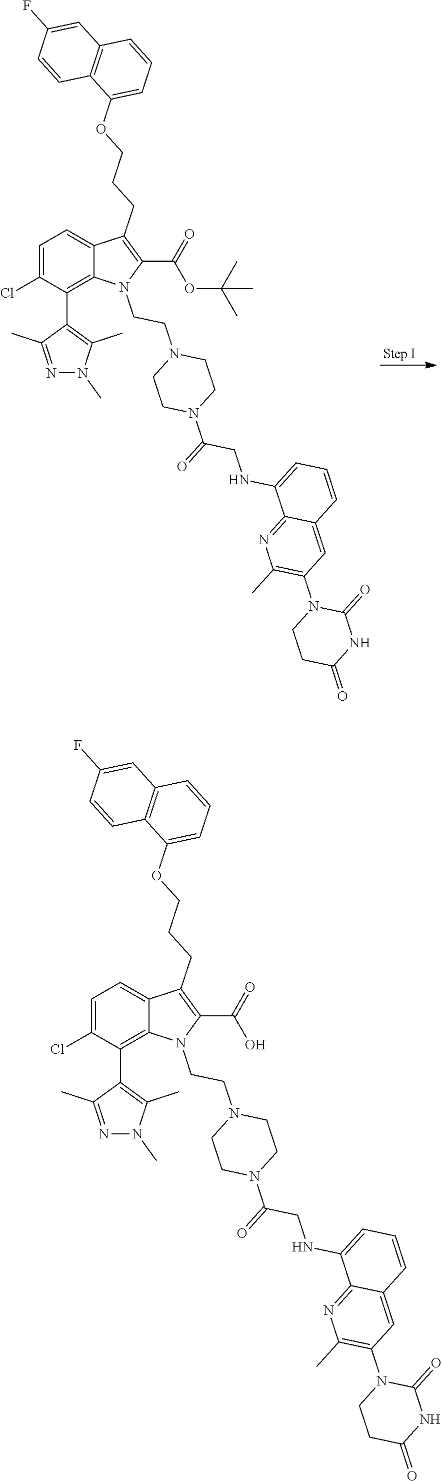 [SMILES: Cc1nc2c(NCC(=O)N3CCN(CCn4c(C(=O)OC(C)(C)C)c(CCCOc5cccc6cc(F)ccc56)c5ccc(Cl)c(-c6c(C)nn(C)c6C)c54)CC3)cccc2cc1N1CCC(=O)NC1=O ; Cc1nc2c(NCC(=O)N3CCN(CCn4c(C(=O)O)c(CCCOc5cccc6cc(F)ccc56)c5ccc(Cl)c(-c6c(C)nn(C)c6C)c54)CC3)cccc2cc1N1CCC(=O)NC1=O]

### Step A

To a solution of 2-amino-3-nitrobenzaldehyde (9.5 g, 57.23 mmol) in n-butanol (400 mL) were added 1-(2-oxopropyl)pyridin-1-ium chloride (10.7 g, 62.95 mmol), DMAP (139 mg, 1.15 mmol) and pyridine (5 mL, 62.95 mmol) under nitrogen. The reaction mixture was stirred at 100° C. for 16 h. Pyrrolidine (11.7 ml, 0.14 mol) was added and the reaction mixture was stirred at 100° C. for additional 2 h. The mixture was cooled to room temperature and concentrated under reduced pressure. The residue was taken up in ethyl acetate and washed with cold water and brine. The organic layer was dried over anhydrous Na₂SO₄ and concentrated under reduced pressure. Crude product was purified by column chromatography (SiO₂, 50% EtOAc in hexane) to get 5 g (24.63 mmol, 43%) of 2-methyl-8-nitroquinolin-3-amine as yellow solid.

LCMS (ESI): 204.2 m/z [M+H]⁺.

### Step B

To a solution of ethyl 3,3-diethoxypropanoate (6.2 mL, 31.99 mmol) in DCM (30 mL) was added TFA (6 mL) at 0° C. under nitrogen. The mixture was stirred at room temperature for 1 h. A solution of 2-methyl-8-nitroquinolin-3-amine (5 g, 24.61 mmol) in DCM (10 mL) was added slowly at 0° C. under nitrogen. The reaction was stirred at room temperature for 45 min and was again cooled to 0° C. Triethylsilane (12 mL, 73.82 mmol) was added dropwise under nitrogen and the solution was stirred at room temperature for 48 h. The reaction was neutralized with saturated NaHCO₃ and extracted with DCM. The organic layer was washed with water and brine, dried over Na₂SO₄ and concentrated under reduced pressure. The residue was purified by column chromatography (SiO₂, 40% EtOAc in DCM) to get 3 g (9.87 mmol, 40%) of ethyl 3-((2-methyl-8-nitroquinolin-3-yl)amino)propanoate as brownish sticky solid.

LCMS (ESI): 303.8 m/z [M+H]⁺.

### Step C

To a solution of ethyl 3-((2-methyl-8-nitroquinolin-3-yl)amino)propanoate (1.5 g, 4.95 mmol) in a mixture of THF (10 mL) and methanol (6 mL), was added a solution of LiOH monohydrate (1 g, 24.73 mmol) in water (2 ml) at 0° C. and the reaction mixture was stirred for 16 h. After completion the mixture was concentrated under reduced pressure. The residue was diluted with water and acidified with 3M HCl. The precipitate was filtered and washed with water to afford 1 g (3.63 mmol, 73%) of 3-((2-methyl-8-nitroquinolin-3-yl)amino)propanoic acid as white solid.

LCMS (ESI): 276.3 m/z [M+H]⁺.

### Step D

To a solution of 3-((2-methyl-8-nitroquinolin-3-yl)amino)propanoic acid (1.5 g, 3.63 mmol) in acetic acid (8 mL), was added urea (5.4 g, 90.91 mmol) at room temperature under nitrogen. The reaction mixture was heated to 100° C. under nitrogen for 24 h. After complete conversion the mixture was concentrated under reduced pressure. The residue was diluted with ethyl acetate, washed successively with aqueous saturated NaHCO₃ and brine. The combined organic layer was dried over Na₂SO₄ and concentrated under reduced pressure to get the crude 1-(2-methyl-8-nitroquinolin-3-yl)dihydropyrimidine-2,4(1H,3H)-dione (500 mg) as light brown sticky solid. Crude product was directly used in the next step without further purification.

LCMS (ESI): 301.2 m/z [M+H]⁺.

### Step E

The solution of crude 1-(2-methyl-8-nitroquinolin-3-yl)dihydropyrimidine-2,4(1H,3H)-dione (500 mg) in DMF (1.5 mL) and ethyl acetate (15 mL) was deoxygenated for 10 min with argon at room temperature. Pd(OH)₂ (150 mg) was then added and the mixture was deoxygenated for additional 5 min. Then the reaction mixture was stirred at room temperature for 5 h under hydrogen atmosphere (balloon pressure). After complete consumption of starting material the reaction mixture was filtered through celite pad and the filtrate was concentrated under reduced pressure. The crude compound was purified by preparative TLC (SiO₂, 5% MeOH in DCM) to afford 106 mg (0.39 mmol, 10% over two steps) of 1-(8-amino-2-methylquinolin-3-yl)dihydropyrimidine-2,4(1H,3H)-dione as off white solid.

LCMS (ESI): 271.0 m/z [M+H]⁺.

### Step F

To 1-(8-amino-2-methylquinolin-3-yl)-1,3-diazinane-2,4-dione (30.0 mg, 0.11 mmol) in DMF (1 mL) was added tert-butyl 2-bromoacetate (18 lpL, 0.12 mmol). The reaction was carried out at 50° C. for 36 h. The reaction mixture was concentrated and the residue was deposited on celite. Crude product was purified by flash column chromatography (SiO₂, 10% MeOH in DCM) to give tert-butyl (3-(2,4-dioxotetrahydropyrimidin-1(2H)-yl)-2-methylquinolin-8-yl)glycinate (35.3 mg, 0.092 mmol, 83%) as yellow oil.

LCMS (ESI): 385.1 m/z [M+H]⁺.

### Step G

To a solution of tert-butyl (3-(2,4-dioxotetrahydropyrimidin-1(2H)-yl)-2-methylquinolin-8-yl)glycinate (35.3 mg, 0.092 mmol) in DCM (1 mL) was added TFA (1 mL, 13.06 mmol). The reaction mixture was stirred at room temperature for 2 h. Then the mixture was concentrated in vacuo and redissolved in 1M HCl aqueous solution. The solution was concentrated to dryness under reduced pressure, redissolved in water and freeze-dried to obtain (3-(2,4-dioxotetrahydropyrimidin-1(2H)-yl)-2-methylquinolin-8-yl)glycine hydrochloride (33.0 mg, 0.09 mmol, 98%) as orange solid.

LCMS (ESI): 329.1 m/z [M+H]⁺.

### Step H

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (15 mg, 0.022 mmol) and (3-(2,4-dioxotetrahydropyrimidin-1(2H)-yl)-2-methylquinolin-8-yl)glycine hydrochloride (16 mg, 0.044 mmol) were dissolved in DMF (1 mL) and DIPEA (23 μL, 0.13 mmol) was added. The reaction mixture was stirred for 30 min at room temperature. HATU (25.4 mg, 0.067 mmol) was added and the reaction was carried out at room temperature for 18 h. The crude product was purified directly by preparative HPLC (C18, H₂O:MeCN+0.1% FA) to afford tert-butyl 6-chloro-1-(2-(4-((3-(2,4-dioxotetrahydropyrimidin-1(2H)-yl)-2-methylquinolin-8-yl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (17.3 mg, 0.017 mmol, 75%) as beige solid.

LCMS (ESI): 984.3 m/z [M+H]⁺.

### Step I

To a solution of tert-butyl 6-chloro-1-(2-(4-((3-(2,4-dioxotetrahydropyrimidin-1(2H)-yl)-2-methylquinolin-8-yl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (17.3 mg, 0.018 mmol) in DCM (1 mL) was added TFA (0.5 mL, 6.53 mmol). The reaction was carried out at room temperature for 18 h. The mixture was concentrated and crude product was purified by preparative HPLC (C18, H₂O:MeCN+0.1% FA) to get 6-chloro-1-(2-(4-((3-(2,4-dioxotetrahydropyrimidin-1(2H)-yl)-2-methyl quinolin-8-yl)glycyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (12 mg, 0.012 mmol, 69%) as beige solid.

LCMS (ESI): 928.2 m/z [M+H]⁺.

¹H NMR (500 MHz, DMSO, 353 K) δ 10.17 (s, 1H), 8.22 (dd, J=9.2, 5.9 Hz, 1H), 8.08 (s, 1H), 7.69 (d, J=8.4 Hz, 1H), 7.56 (dd, J=10.3, 2.7 Hz, 1H), 7.44-7.37 (m, 2H), 7.36-7.28 (m, 2H), 7.20 (d, J=8.4 Hz, 1H), 7.05 (d, J=8.2 Hz, 1H), 6.90-6.82 (m, 2H), 6.66 (d, J=7.7 Hz, 1H), 4.33-4.25 (m, 1H), 4.23 (t, J=6.3 Hz, 2H), 4.21-4.14 (m, 1H), 4.04 (d, J=4.3 Hz, 2H), 3.86-3.77 (m, 1H), 3.75 (s, 3H), 3.44-3.40 (m, 4H), 2.81 (s, 2H), 2.57 (s, 3H), 2.26-2.20 (m, 2H), 2.17-2.12 (m, 4H), 2.12-2.07 (m, 2H), 2.07-2.02 (m, 1H), 2.00 (s, 3H), 1.88 (s, 3H).

The signals of two aliphatic protons overlap with water signal.

### Example 29: Fluorescence Polarization (FP) Assays

To study molecular interactions between compounds of the invention and MCL-1 as well as cereblon, a fluorescence polarization assay was used.

### Fluorescence Polarization (FP) Assay: CRBN

CRBN-DDB1 protein complex was mixed with Cy5-labelled thalidomide and a compound to be tested. The test solution contained 50 mM Tris pH=7.0, 200 mM NaCl, 0.02% v/v Tween-20, 2 mM DTT, 5 nM Cy5-labelled thalidomide (the tracer), 25 nM CRBN-DDB1 protein, 2% v/v DMSO. The test solution was added to a 384-well assay plate.

The plate was spun-down (1 min, 1000 rpm, 22° C.) and then shaken using a VibroTurbulator for 30 seconds at room temperature (20-25° C.), with the frequency set to level 10. The assay plate with protein and the tracer was incubated for 60 min at room temperature (20-25° C.) prior to read-out with a plate reader. Read-out (fluorescence polarization) was performed by a Pherastar plate reader, using a Cy5 FP Filterset (590 nm/675 nm).

The FP experiment was carried out with various concentrations of the test compounds in order to measure Ki values.

The Ki values of competitive inhibitors were calculated using the equation based on the IC₅₀ values of relationship between compound concentration and measured fluorescence polarization, the Kd value of the Cy5-T and CRBN/DDB1 complex, and the concentrations of the protein and the tracer in the displacement assay (as described by Z. Nikolovska-Coleska et al., Analytical Biochemistry 332 (2004) 261-273).

### Fluorescence Polarization (FP) Assay—Results

Compounds are categorized based on their affinity to CRBN defined as Ki.

CRBN binding Ki [μM] is indicated as follows:

$${A}_{C R B N} < 1 μM$$

$$1 μM \leq {B}_{C R B N} \leq 5 μM$$

$${C}_{C R B N} > 5 μM$$

### Fluorescence Polarization (FP) Assay: MCL-1

MCL-1 protein was mixed with the tracer—FI-392—and a compound to be tested. The test solution contained 20 mM Tris pH=7.5, 50 mM NaCl, 0.02% v/v Tween-20, 3 mM DTT, 2 nM tracer, 20 nM, MCL-1 protein, 5% v/v DMSO. The test solution was added to a 384-well assay plate.

The plate was spun down (1 min, 1000 rcf, 22° C.) and then shaken using a VibroTurbulator for 30 sec at room temperature (20-25° C.), with the frequency set to level 10. The assay plate with protein and the tracer was incubated for 60 min at room temperature (20-25° C.) prior to read-out with a plate reader. Read-out (fluorescence polarization) was performed by a Pherastar plate reader, using a FP Filterset (485 nm/520 nm).

The FP experiment was carried out with various concentrations of the test compounds in order to measure Ki values.

The Ki values of competitive inhibitors were calculated using the equation based on the IC₅₀ values of relationship between compound concentration and measured fluorescence polarization, the Kd value of the tracer and MCL-1 protein, and the concentrations of the protein and the tracer in the displacement assay (as described by Z. Nikolovska-Coleska et al., Analytical Biochemistry 332 (2004) 261-273).

### Fluorescence Polarization (FP) Assay—Results

Compounds are categorized based on their affinity to MCL-1 defined as Ki.

MCL-1 binding Ki [μM] is indicated as follows:

$${A}_{MCL - 1} < 0.01 μM$$

$$0.01 μM \leq {B}_{MCL - 1} \leq 0.1 μM$$

$${C}_{MCL - 1} > 0.1 μM$$

TABLE 9
Fluorescence Polarization (FP) Assay

| Compound ID | CRBN binding Ki [μM] | MCL-1 binding Ki [μM] |
|---|---|---|
| 1 | C_{CRBN} | A_{MCL-1} |
| 2 | B_{CRBN} | A_{MCL-1} |
| 3 | A_{CRBN} | A_{MCL-1} |
| 4 | C_{CRBN} | A_{MCL-1} |
| 5 | B_{CRBN} | A_{MCL-1} |
| 6 | B_{CRBN} | A_{MCL-1} |
| 7 | B_{CRBN} | A_{MCL-1} |
| 10 | B_{CRBN} | A_{MCL-1} |
| 11 | B_{CRBN} | A_{MCL-1} |
| 12 | B_{CRBN} | A_{MCL-1} |
| 13 | B_{CRBN} | A_{MCL-1} |
| 14 | N | A_{MCL-1} |
| 15 | C_{CRBN} | A_{MCL-1} |
| 21 | A_{CRBN} | A_{MCL-1} |
| 22 | A_{CRBN} | A_{MCL-1} |
| 23 | A_{CRBN} | A_{MCL-1} |
| 24 | N | A_{MCL-1} |
| 25 | N | A_{MCL-1} |
| 26 | A_{CRBN} | A_{MCL-1} |
| 27 | A_{CRBN} | A_{MCL-1} |
| 28 | A_{CRBN} | A_{MCL-1} |
| 29 | B_{CRBN} | A_{MCL-1} |
| 36 | C_{CRBN} | A_{MCL-1} |
| 144 | B_{CRBN} | A_{MCL-1} |
| 145 | A_{CRBN} | A_{MCL-1} |
| 146 | B_{CRBN} | A_{MCL-1} |
| 147 | C_{CRBN} | A_{MCL-1} |
| 148 | C_{CRBN} | A_{MCL-1} |

### Example 30: Ternary Complex Formation Assay

The effect of the bifunctional compounds of the invention on the formation of a ternary complex composed of [MCL-1]-[compound of formula (I)]—[CRBN/DDB1] was investigated with HTRF ternary complex assay.

Mix of protein solution was prepared:
48 nM hMcl-1 and 105.6 nM CRBN were prepared in PPI Europium detection buffer with 2 mM DTT.
Acceptor and donor stock solutions were diluted 100 times in PPI Europium detection buffer. Equal volumes of both reagents were mixed. Finally giving 3 nM of Streptavidin-Eu cryptate (acceptor) and 6.67 nM of Anti-6×his-d2 (donor).

5 μl of protein mix was dispensed to all wells. The tested compounds in dose-response were dispensed onto a white 384-well low volume plate (Greiner, 784075). DMSO was backfilled to all wells, resulting in a final DMSO content of 0.5%. Wells containing only DMSO served as a background. Next, 5 μl of solution with donor and acceptor was added to the wells.

The plate was sealed with transparent film and shaken using a VibroTurbulator for 60 sec at level 3. The plate was then spun down shortly (10 s, 1000 rcf) and incubated at 25° C. for 180 min.

The read-out was performed with plate reader (Pherastar, BMG Labtech) in time resolved fluorescence mode. Filterset: TR 337 665 620.

The results were analyzed as follows:
1) an average of fluorescence for background signal was calculated and used as a negative control;
2) raw fluorescence values for tested compounds were normalized against negative controls;
3) bell shape curve was fitted to a gaussian distribution using a non-linear least squares Gauss-Newton algorithm;
4) EC₅₀ and pEC₅₀ values were determined.

As illustrated in Table 10, the compounds of the present invention have the capability to induce the formation of the [MCL-1]-[compound of formula (I)]—[CRBN/DDB1] complex.

TABLE 10
Ternary complex assay results for the compounds of the invention

| | HTRF ternary complex |
|---|---|
| Compound ID | Average pEC₅₀ by molecule |
| 1 | >7.0 |
| 2 | >7.0 |
| 3 | >7.0 |
| 4 | >7.0 |
| 5 | >7.0 |
| 6 | >7.0 |
| 7 | >7.0 |
| 10 | >6.5 |
| 11 | >6.5 |
| 12 | >6.5 |
| 13 | >6.5 |
| 14 | >7.5 |
| 15 | >6.5 |
| 21 | >7.5 |
| 22 | >7.0 |
| 23 | >6.5 |
| 24 | N |
| 25 | N |
| 26 | >7.0 |
| 27 | >7.0 |
| 28 | >7.0 |
| 29 | >7.0 |
| 36 | >6.5 |
| 144 | >7.5 |
| 145 | >7.5 |
| 146 | >7.5 |
| 147 | >7.0 |
| 148 | >7.5 |

### Example 31: Cell Viability Assays MV4-11, ARH-77 Cells

The effect of various compounds of the invention on the viability of MV4-11 and ARH-77 cell lines was investigated, using the CTG protocol described below.

MV4-11 cell lines are derived from acute myeloid leukaemia and it is described in the literature as a MCL-1 dependent cell line, as opposed to lymphoblast-like ARH-77 cell line, which is described as independent of MCL-1 (Tron A E et al. Nat Commun. 2018; 9: 5341; Caenepeel S et al. Cancer Discov. 2018 December; 8(12): 1582-1597).

Cells (MV4-11 or ARH-77) were seeded on 348-well plates in a growth medium with 1% FBS. Next, compound stocks were pre-diluted in DMSO and added directly to the cell culture media (final DMSO concentration: 0.5%). Compound concentrations used in this assay: 1/3 dilutions starting at 30 μM (9 to 12 points). After 24 hours of incubation, cell viability was evaluated using CellTiter-Glo Luminescent Cell Viability Assay (Promega, cat #G7572). The luminescence signal was measured using a CLARIOstar Plus Multi-Mode Microplate Reader (574-590 nm).

Results were normalized to DMSO control. Collected data were analyzed using GraphPad Prism to calculate pCC₅₀ values.

TABLE 11
The effect of various compounds of the invention
on the viability of MV4-11 and ARH-77 cell lines

| | Average pCC₅₀ | Average pCC₅₀ |
|---|---|---|
| | by molecule | by molecule |
| Compound ID | MV4-11 | ARH-77 |
| 1 | >7.0 | <5.0 |
| 2 | >7.0 | <5.0 |
| 3 | >7.0 | <4.5 |
| 4 | >6.5 | <5.0 |
| 5 | Not determined | <5.0 |
| 6 | >7.0 | <5.0 |
| 7 | >7.0 | <5.0 |
| 10 | >6.5 | <5.0 |
| 11 | >7.0 | <5.0 |
| 12 | >7.0 | <4.5 |
| 13 | Not determined | <5.0 |
| 14 | >6.5 | <5.5 |
| 15 | >7.0 | <5.0 |
| 21 | >6.5 | <5.0 |
| 22 | >7.5 | <4.5 |
| 23 | >7.5 | <5.0 |
| 24 | Not determined | <5.0 |
| 25 | Not determined | <5.5 |
| 26 | Not determined | <4.5 |
| 28 | >6.5 | <5.0 |
| 29 | Not determined | <5.0 |
| 36 | >7.0 | <5.0 |
| 144 | >7.0 | <5.5 |
| 145 | >7.0 | <5.0 |
| 146 | >7.0 | Not determined |
| 147 | >7.0 | <4.5 |
| 148 | >6.5 | <5.5 |
| dMCL-1-2 | >6.0 | <4.5 |
| (Papatzimas et al., 2019) | | |

As illustrated in Table 11, the compounds of the present invention have the capability to decrease the viability of acute myeloid leukaemia cells, and thus can be useful in the treatment of cancer. Simultaneously, the compounds of the present invention have no/little impact on the viability of ARH-77, which further supports a specific, MCL-1 dependent activity of said compounds. dMCL-1-2 is MCL-1 degrader described in Papatzimas et al., J. Med. Chem. 2019, 62, 5522-5540.

### Abbreviations and Definitions

A list of the abbreviations used in the present application is shown in Table 12, below:

TABLE 12
Abbreviations

| Abbreviation | Meaning |
|---|---|
| CRBN | Cereblon |
| DDB1 | damaged DNA binding protein 1 |
| TFA | trifluoroacetic acid |
| CDI | carbonyldiimidazole |
| FA | formic acid |
| FBS | fetal bovine serum |
| MsCl | methanesulfonyl chloride |
| Tris | Tris(hydroxymethyl)aminomethane |
| DMSO | Dimethylsulfoxide |
| THF | tetrahydrofuran |
| DMF | dimethylformamide |
| DMA | dimethylacetamide |
| DMAP | 4-dimethylaminopyridine |
| EDC | 1-ethyl-3-(3-dimethylaminopropyl)carbodiimide |
| HPLC | High performance liquid chromatography |
| NCS | N-chlorosuccinimide |
| DIPEA | N,N-diisopropylethylamine |
| HATU | Hexafluorophosphate Azabenzotriazole |
| | Tetramethyl Uronium |
| | (1-[Bis(dimethylamino)methylene]-1H- |
| | 1,2,3-triazol[4,5-b]pyridinium-2-oxide |
| | hexafluorophosphate) |
| m-CPBA | meta-chloroperbenzoic acid |
| Pd(dppf)Cl₂ | [1,1′-bis(diphenyl- |
| CH₂Cl₂ | phosphino)ferrocene]dichloropalladium(II), |
| | complex with dichloromethane |
| DTT | dithiothreitol |
| RT | Room temperature |

As used herein, the term “room temperature” means a temperature of between 20° C. and 25° C.

The invention is further described with reference to the following clauses:

1. A compound of formula (I)

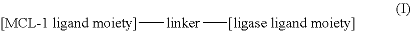

or a salt, solvate, hydrate, isomer or prodrug thereof,
wherein [ligase ligand moiety] is:

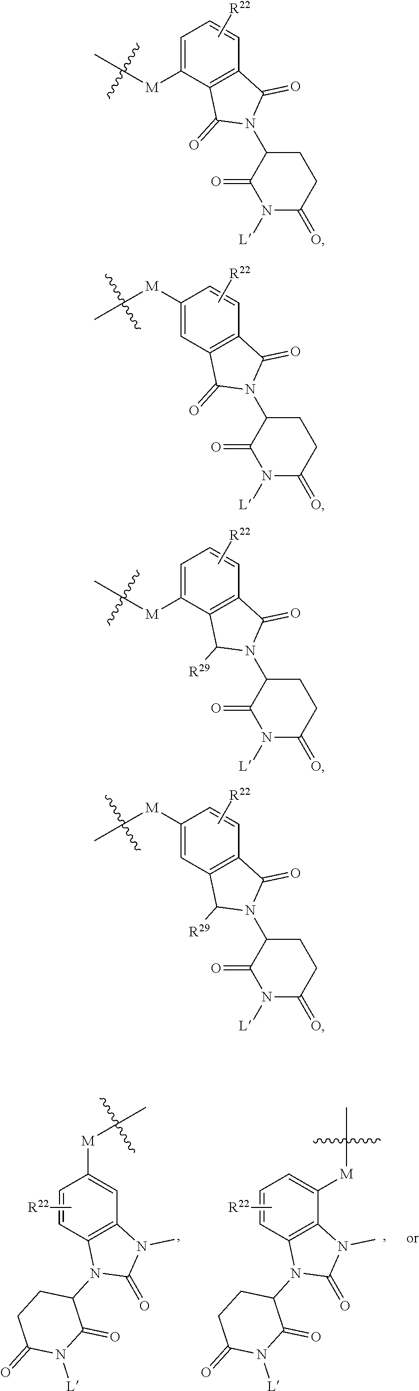

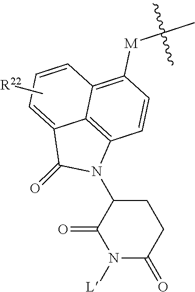

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   R²⁹ is hydrogen or Me
   L′ is hydrogen, alkyl, benzyl, acetyl or pivaloyl;
wherein [MCL-1 ligand moiety] is a compound of Formula (A1), (A2), (A3) or (A4):

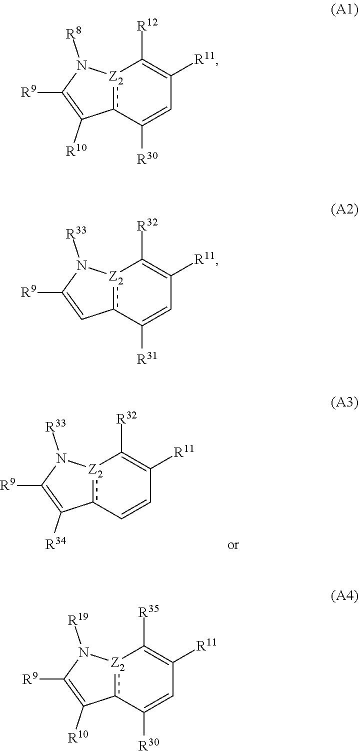

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline,
wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹² is

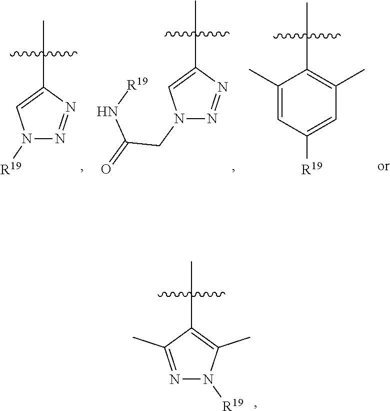

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C—C₆ alkyl); or wherein the tetraline is optionally substituted with a bridging —CH₂— group; or wherein the naphthyl is optionally substituted with —O— or —S—;
each R³² is independently H,

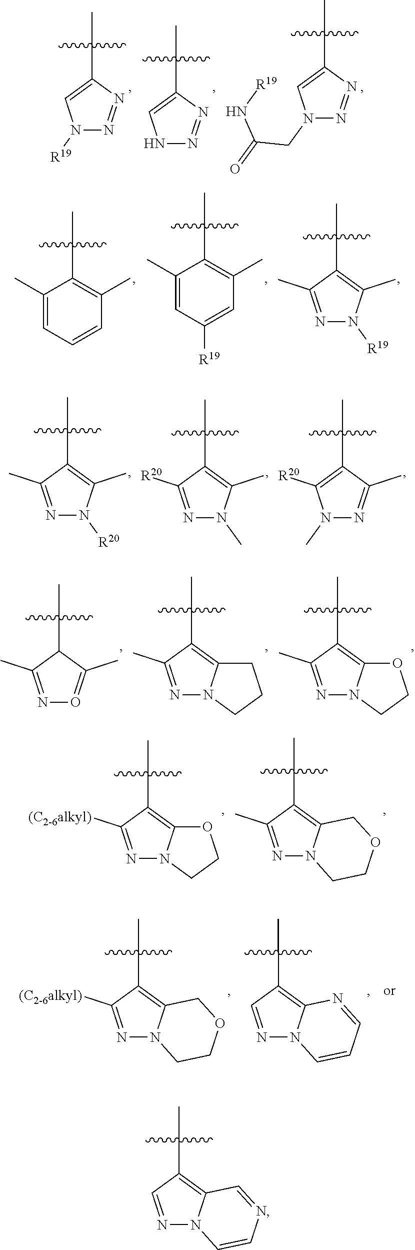

wherein R²⁰ is Me, —CH₂—OMe, —CH₂—O-bromobenzaldehyde, or

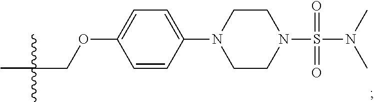 [SMILES: *COc1ccc(N2CCN(S(=O)(=O)N(C)C)CC2)cc1]

R³⁴ is —O—C₂₋₆ alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³ is

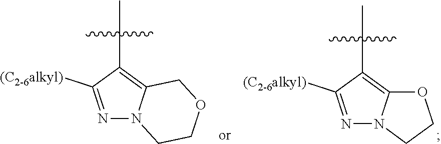

and
R¹⁹ is a bond connected to R¹⁴ of the linker, wherein each of Formula (A1), Formula (A2), Formula (A3) and Formula (A4) contains a single R¹⁹;
and wherein [linker] has the following formula

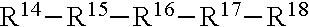

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is —C₁₋₆ alkyl, cycloalkyl, CH₂—NH—C(O)—, heterocycloalkyl, or is absent
   wherein at least one of R¹⁴-R¹⁸ is present.

2. The compound of clause 1, wherein [ligase ligand moiety] is:

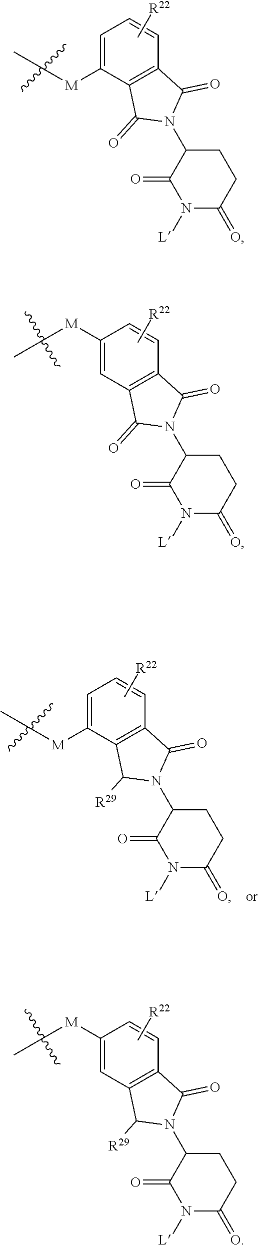

3. The compound of clause 1 or 2, wherein R²⁹ is Me.

4. The compound of clause 1 or 2, wherein R²⁹ is hydrogen.

5. The compound of clause 1, wherein [ligase ligand moiety] is:

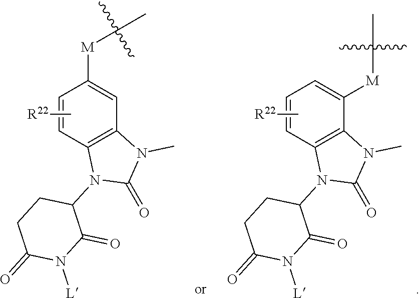

6. The compound of clause 1, wherein [ligase ligand moiety] is:

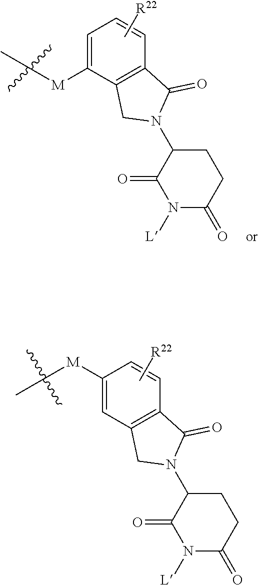

7. The compound of any preceding clause, wherein R²² is hydrogen, halogen, —OMe, —NH₂, —NHMe, —NMe₂, or piperidine; optionally wherein R²² is hydrogen, —OMe, —NH₂, —NHMe, —NMe₂, or piperidine.

8. The compound of clause 7, wherein R²² is hydrogen.

9. The compound of any preceding clause, wherein L′ is hydrogen.

10. The compound of any preceding clause, wherein M is O or NH, or is absent.

11. The compound of clause 1, wherein [ligase ligand moiety] is

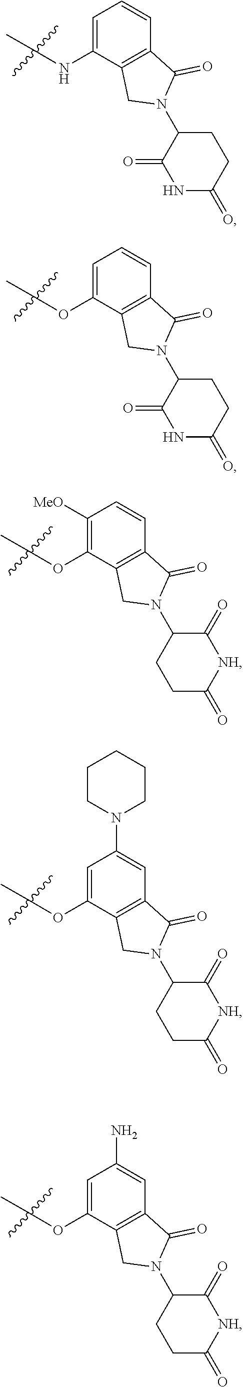 [SMILES: *Nc1cccc2c1CN(C1CCC(=O)NC1=O)C2=O ; *Oc1cccc2c1CN(C1CCC(=O)NC1=O)C2=O ; *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cc(N)cc2c1CN(C1CCC(=O)NC1=O)C2=O]

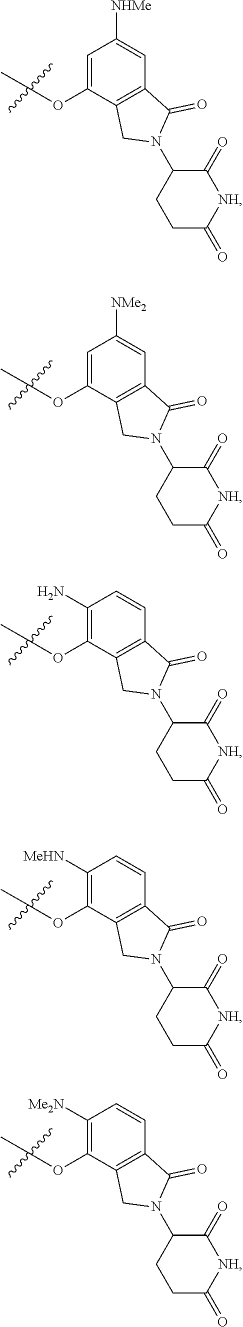 [SMILES: *Oc1cc(NC)cc2c1CN(C1CCC(=O)NC1=O)C2=O ; *Oc1cc(N(C)C)cc2c1CN(C1CCC(=O)NC1=O)C2=O ; *Oc1c(N)ccc2c1CN(C1CCC(=O)NC1=O)C2=O ; *Oc1c(NC)ccc2c1CN(C1CCC(=O)NC1=O)C2=O ; *Oc1c(N(C)C)ccc2c1CN(C1CCC(=O)NC1=O)C2=O]

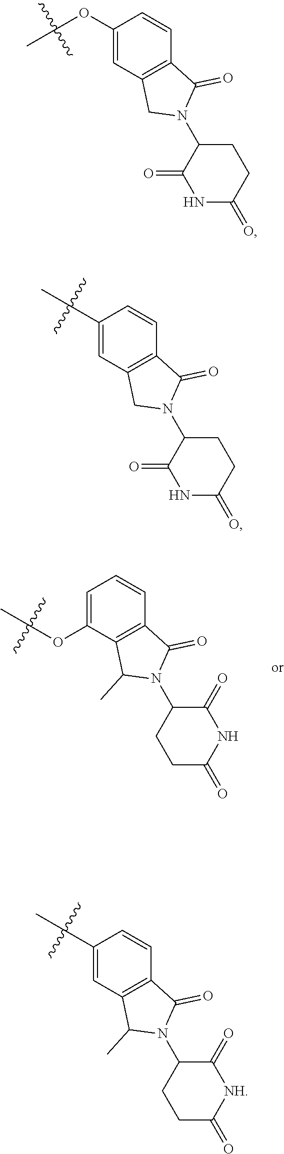 [SMILES: *Oc1ccc2c(c1)CN(C1CCC(=O)NC1=O)C2=O ; *c1ccc2c(c1)CN(C1CCC(=O)NC1=O)C2=O ; *Oc1cccc2c1C(C)N(C1CCC(=O)NC1=O)C2=O ; *c1ccc2c(c1)C(C)N(C1CCC(=O)NC1=O)C2=O]

12. The compound of clause 1, wherein [ligase ligand moiety] is

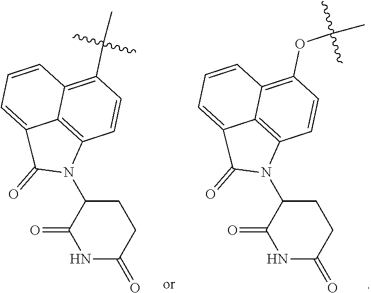 [SMILES: *c1ccc2c3c(cccc13)C(=O)N2C1CCC(=O)NC1=O ; *Oc1ccc2c3c(cccc13)C(=O)N2C1CCC(=O)NC1=O]

13. The compound of clause 1, wherein [ligase ligand moiety] is:

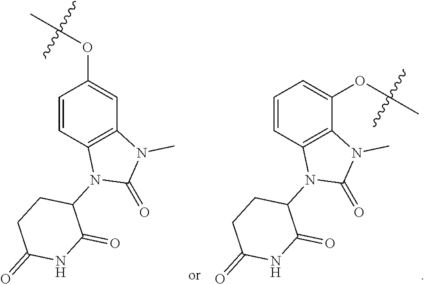 [SMILES: *Oc1ccc2c(c1)n(C)c(=O)n2C1CCC(=O)NC1=O ; *Oc1cccc2c1n(C)c(=O)n2C1CCC(=O)NC1=O]

14. The compound of any preceding clause, wherein R¹⁴ is —C₁₋₆ alkyl, —C₂₋₆ alkenyl, —C₂₋₆ alkynyl, —C(O)—, —SO₂— or is absent.

15. The compound of clause 14, wherein R¹⁴ is —C₁₋₆ alkyl.

16. The compound of any preceding clause, wherein R⁵ is cycloalkyl, heterocycloalkyl, aryl, heteroaryl, —C₁₋₆ alkyl-NH—, -cycloalkyl-NH— or is absent.

17. The compound of clause 16, wherein R¹⁵ is heterocycloalkyl or is absent.

18. The compound of clause 17, wherein R¹⁵ is piperazine, bridged piperazine, piperazine N-oxide, piperazine cation,

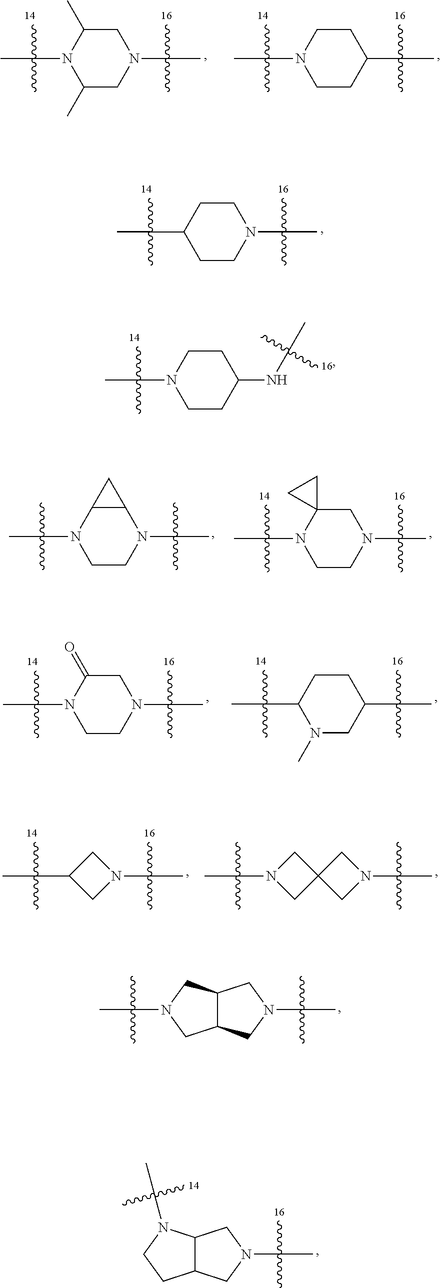

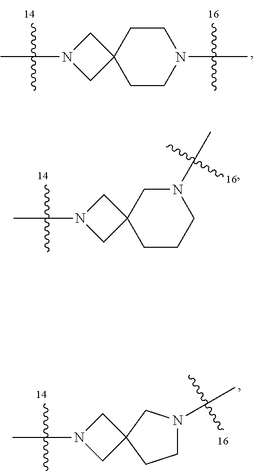 [SMILES: *N1CCC2(CC1)CN(*)C2 ; *N1CCCC2(C1)CN(*)C2 ; *N1CCC2(C1)CN(*)C2]

or is absent;
wherein [symbol] indicates attachment to R¹⁴ and [symbol] indicates attachment to R¹⁶.

19. The compound of clause 18, wherein R⁵ is piperazine,

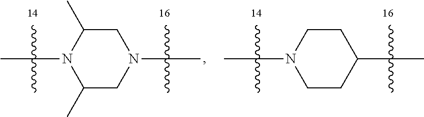

or is absent.

20. The compound of any preceding clause, wherein R¹⁶ is —C₁₋₆ alkyl, —CH₂—C(O)—NH—, —CH₂—C(O)—, —C(O)— or is absent.

21. The compound of any preceding clause, wherein R¹⁷ is absent.

22. The compound of any preceding clause, wherein R¹⁸ is —C₁₋₆ alkyl, cyclobutyl, CH₂—NH—C(O)—, piperazine or is absent.

23. The compound of any preceding clause, wherein [linker] is selected from

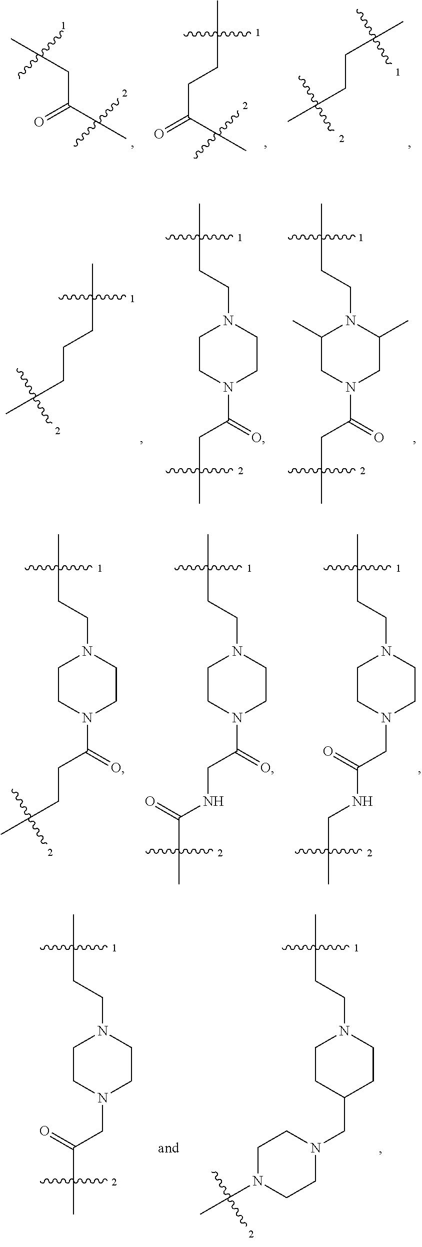

wherein
   [symbol] indicates attachment to [MCL-1 ligand moiety] and
   [symbol] indicates attachment to [ligase ligand moiety].

24. The compound of clause 23, wherein [linker] is selected from

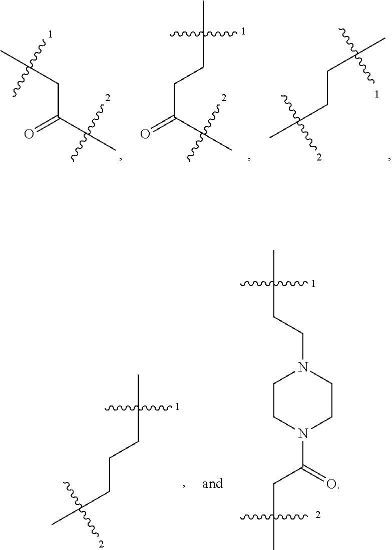

25. The compound of any preceding clause, wherein [MCL-1 ligand moiety] is of Formula (A1):

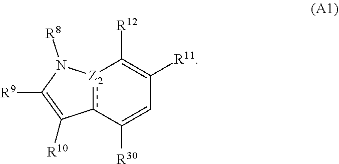

26. The compound of any one of clauses 1-24, wherein [MCL-1 ligand moiety] is of Formula (A2):

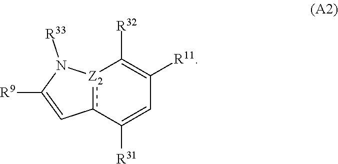

27. The compound of any one of clauses 1-24, wherein [MCL-1 ligand moiety] is of Formula (A3):

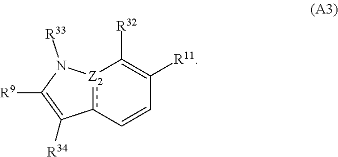

28. The compound of any one of clauses 1-24, wherein [MCL-1 ligand moiety] is of Formula(A4):

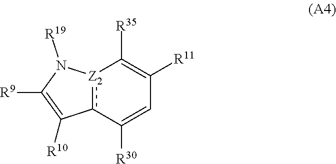

29. The compound of any preceding clause wherein in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R³, wherein R¹³ is phenyl or naphthyl, wherein the phenyl or naphthyl is optionally substituted with at least one substituent selected from halogen and C₁-C₆ alkyl.

30. The compound of any preceding clause, wherein in Formula (A1) R¹² is

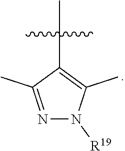

31. The compound of any preceding clause, wherein in Formula (A4) R³⁵ is

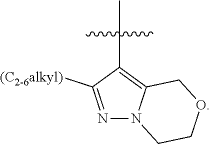

32. The compound of any preceding clause, wherein in Formula (A2) R³¹ is —C₂₋₅alkyl-O—R¹³ or —O—C₂₋₅alkyl-R³, wherein R¹³ is phenyl or naphthyl, wherein the phenyl or naphthyl is optionally substituted with at least one substituent selected from halogen and C₁-C₆ alkyl.

33. The compound of any preceding clause, wherein in Formula (A3) R³⁴ is —O—C₂₋₅alkyl-R¹³ wherein R¹³ is phenyl or naphthyl, wherein the phenyl or naphthyl is optionally substituted with at least one substituent selected from halogen and C₁-C₆ alkyl.

34. The compound of any preceding clause, wherein Z₂ is C and [symbol] is a double bond.

35. The compound of any preceding clause, wherein each R⁹ is independently —C(O)OH or —P(O)(OH)₂.

36. The compound of any preceding clause, wherein R¹¹ is halogen.

37. The compound of any preceding clause, wherein each R²⁰ is Me.

38. The compound of any preceding clause, wherein [MCL-1 ligand moiety] is selected from:

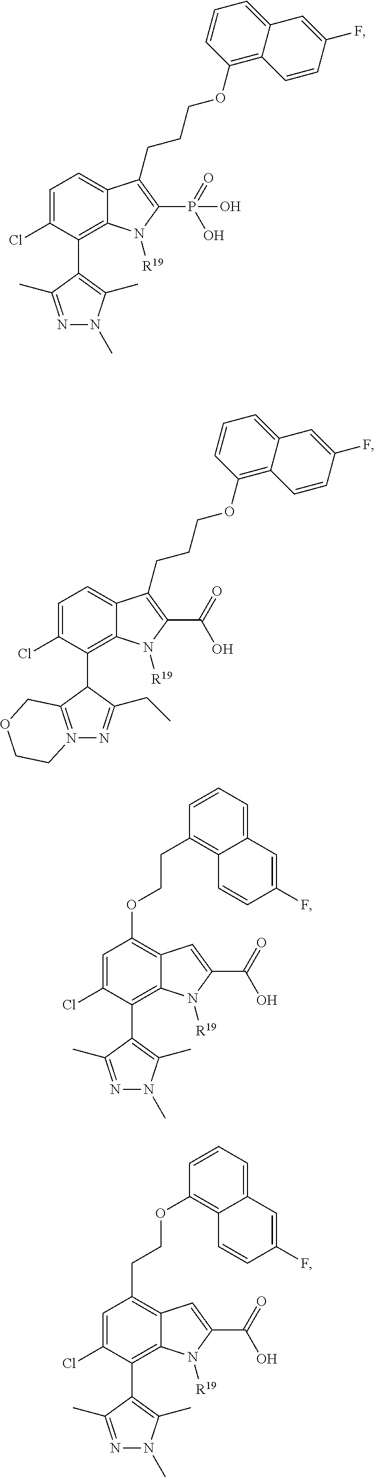

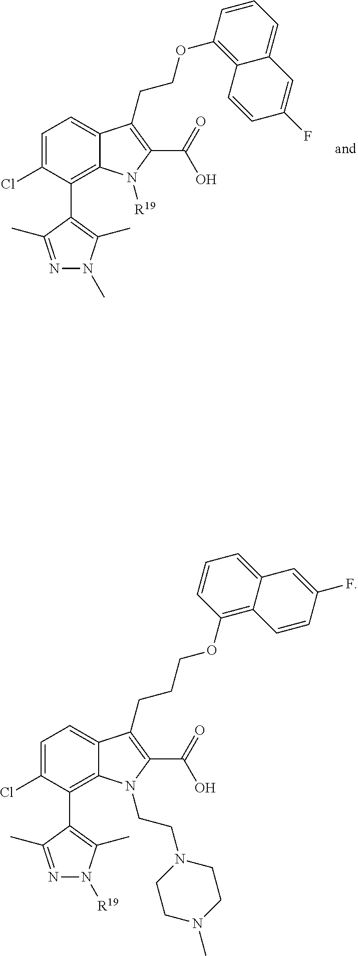

39. The compound of clause 1, which is

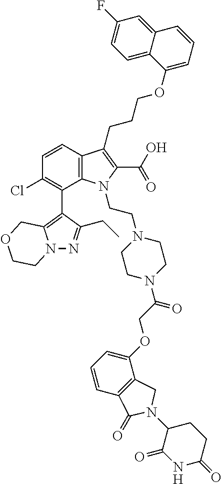 [SMILES: CCc1nn2c(c1-c1c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c13)COCC2]

40. A compound of formula (I)

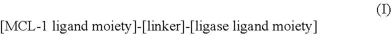

or a salt, solvate, hydrate, isomer or prodrug thereof,
wherein [ligase ligand moiety] is:
   (a) Formula (IV)

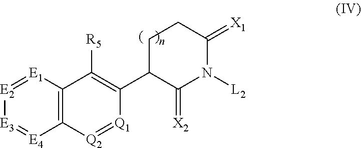

wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
and
each R^{b} is independently hydrogen, alkyl, alkenyl, aryl, heteroaryl, or benzyl;
wherein R²¹ is a bond connected to R¹⁸ of the linker, and wherein Formula (IV) contains a single R²¹;
   (b) Formula (VIIa), (VIIb), or (VIIc):

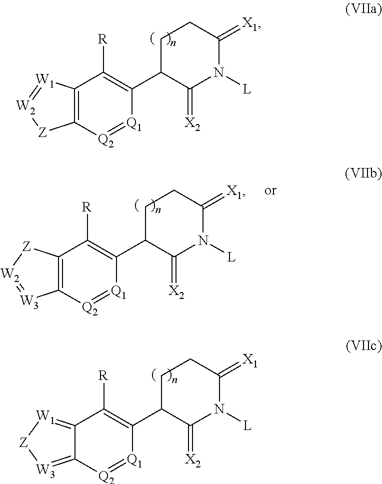

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR^{e};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹; each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII):

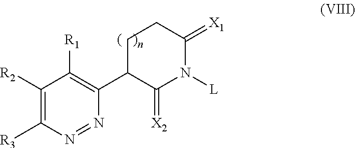

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX):

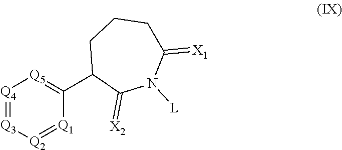

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single
R²¹;
wherein [MCL-1 ligand moiety] is a compound of Formula (A1), (A2), (A3) or (A4):

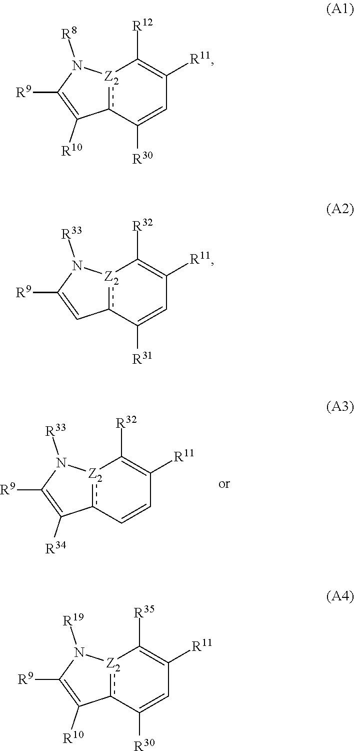

wherein
   [symbol] is a single bond or a double bond;
   each Z₂ is independently N or C, wherein when Z₂ is N, then [symbol] is a single bond; and when Z₂ is C, [symbol] then is a double bond;
   each R⁹ is independently —C(O)OH, —C(O)OC₁-C₆alkyl, —C(O)NH₂ or —P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,

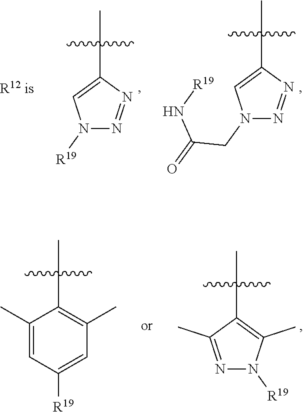

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

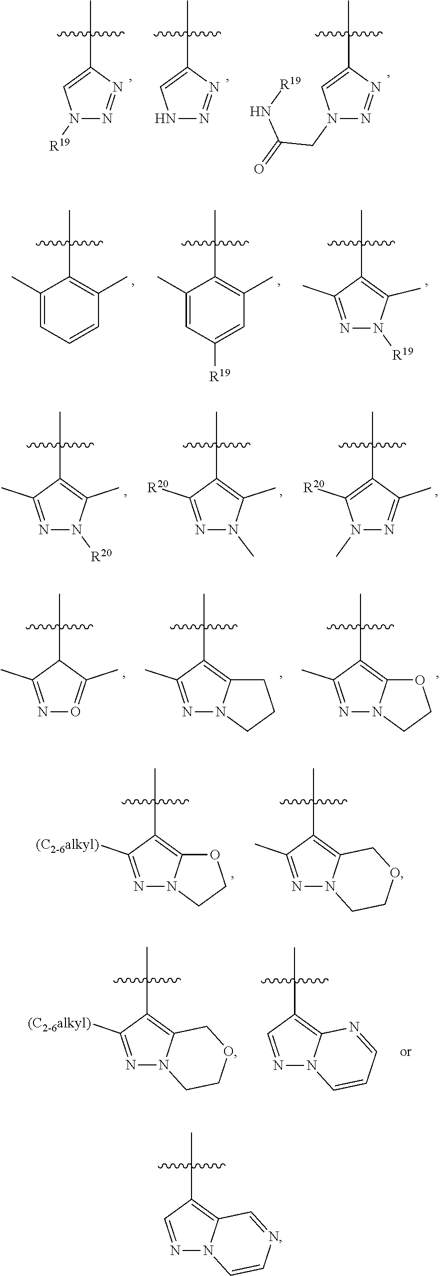

wherein R²⁰ s Me, —CH₂—OMe, —CH₂—O-bromobenzaldehyde, or;

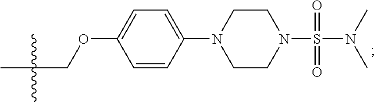 [SMILES: *COc1ccc(N2CCN(S(=O)(=O)N(C)C)CC2)cc1]

R³⁴ is —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is or

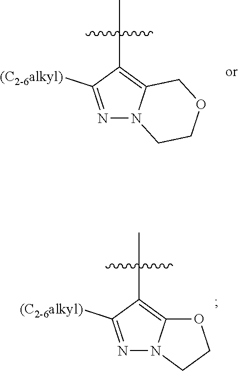

and
R¹⁹ is a bond connected to R¹⁴ of the linker, wherein each of Formula (A1), Formula (A2), Formula (A3) and Formula (A4) contains a single R¹⁹;
and wherein [linker] has the following formula

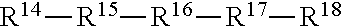

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is —C₁₋₆ alkyl, cycloalkyl, CH₂—NH—C(O)—, heterocycloalkyl, or is absent
   wherein at least one of R¹⁴-R¹⁸ is present.

41. A compound of formula (I)

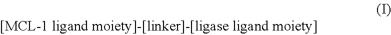

or a salt, solvate, hydrate, isomer or prodrug thereof,
wherein [ligase ligand moiety] is:
   (a) Formula (Va) or (Vb):

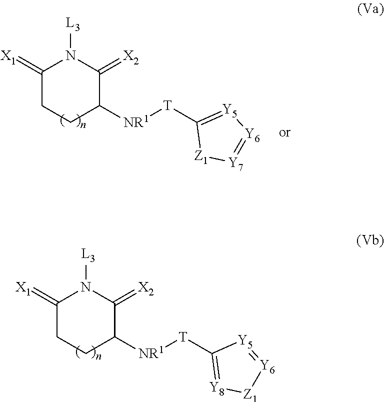

or a pharmaceutically acceptable salt or tautomer thereof,
   wherein
   each of X₁ and X₂ is independently O or S;
   Z₁ is O, S or NR⁶;
   T is is C═O or SO₂;
   R¹ is hydrogen, alkyl, cycloalkyl, alkenyl, cycloalkenyl, aryl, heteroaryl, or benzyl; each of Y₅, Y₆, Y₇, and Y₈ is independently N or CR⁷,
   wherein at least one of Y₅, Y₆ and Y₇ in Formula (Va) is CR⁷, and at least one of Y₅, Y₅ and Y₅ in Formula (Vb) is CR⁷;
   n is 0, 1 or 2;
   L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
   each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
   each R″″ is independently hydrogen, alkyl, cycloalkyl, alkenyl, cycloalkenyl, aryl, heteroaryl, or benzyl;
   R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
   wherein R²¹ is a bond connected to R¹⁸ of the linker, and wherein formula (Va) and formula (Vb) each contain a single R²¹;
   wherein when Z₁ is O, then Y₆ is CR⁷ and
wherein when the compound is of Formula (Va), then
   (i) when each of Y₅, Y₆ and Y₇ is CR⁷, then at least one of R⁷ is not H;
   (ii) when Z₁ is NR⁶, then Y₆ and Y₇ are CR⁷;
   (iii) when Z₁ is S, then Y₅ is not C—OMe and Y₆ is not C—OMe;
   (iv) when Z₁ is S and Y₅ is C—NHCOMe, then Y₇ is not C—CH₂NR″″C(O)OR″″;
   (v) when Z₁ is S and Y₅ is N, then Y₆ is not C—H, C-aryl or C—C(O)OR″″; and
   (vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
   (vii) when each of Y₅, Y₆ and Y₈ is CR⁷, then at least one of R⁷ is not H;
   (viii) when Z₁ is S, then Y₅ is not C—COOH or C—NHC(O)Me, and Y₈ is not C—Br;
   (ix) when Z₁ is S and Y₆ is C—Br, then Y₈ is C—OR″″
   (x) when Z₁ is S, Y₅ is N and Y₆ is C—H or C—NH₂, then Y₈ is not C—H
   (xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷. or
   (b) Formula (IIa) or (IIb):

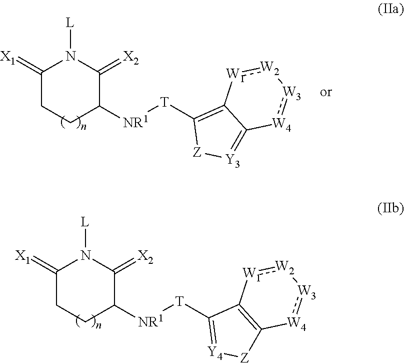

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR^{a}, wherein at least one of W₁, W₂, W₃ and W₄ is N, and
   when each [symbol] is a single bond, W₁, W₂, W₃ and W₄ are each CR^{a2} and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 ligand moiety] is a compound of Formula (A1), (A2), (A3) or (A4):

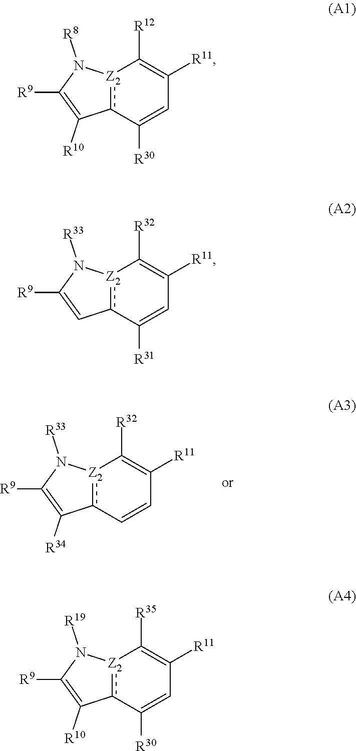

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or —P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline,
wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹² is

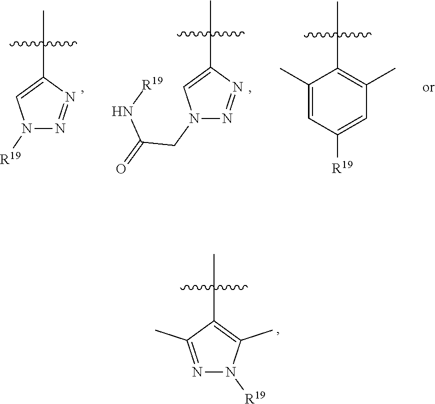

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

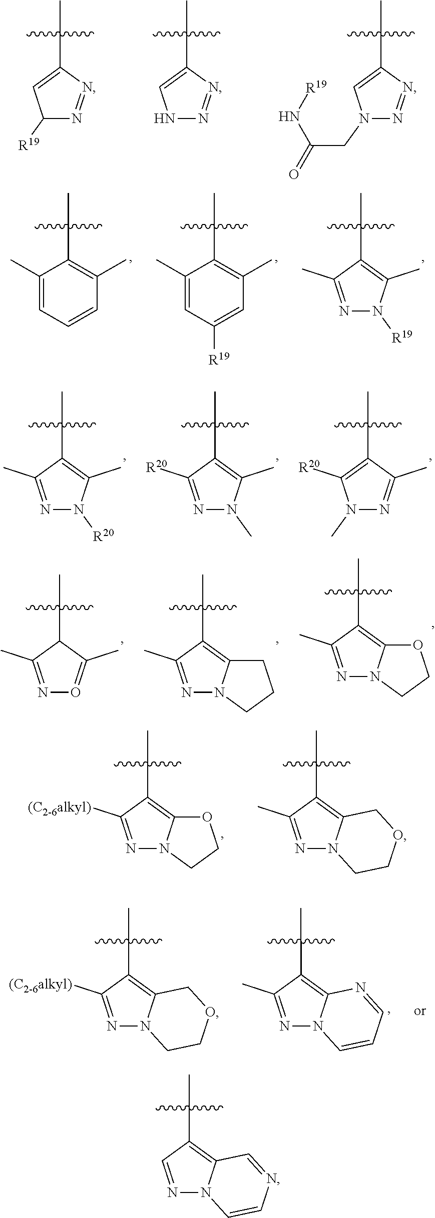

wherein R²⁰ is Me, —CH₂—OMe, —CH₂—O-bromobenzaldehyde, or

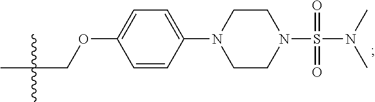 [SMILES: *COc1ccc(N2CCN(S(=O)(=O)N(C)C)CC2)cc1]

R³⁴ is —O—C₂₋₆ alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

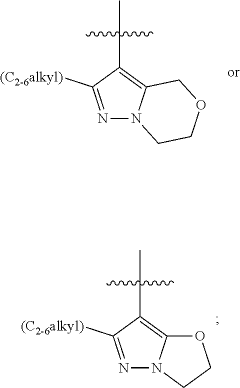

and
R¹⁹ is a bond connected to R¹⁴ of the linker, wherein each of Formula (A1), Formula (A2), Formula (A3) and Formula (A4) contains a single R¹⁹;
and wherein [linker] has the following formula

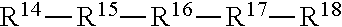

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is —C₁₋₆ alkyl, cycloalkyl, CH₂—NH—C(O)—, heterocycloalkyl, or is absent
   wherein at least one of R¹⁴-R¹⁸ is present.

42. A compound of formula (I)

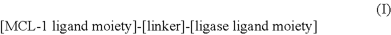

or a salt, solvate, hydrate, isomer or prodrug thereof,
wherein [ligase ligand moiety] is:
   (a) Formula (VIa) or (VIb):

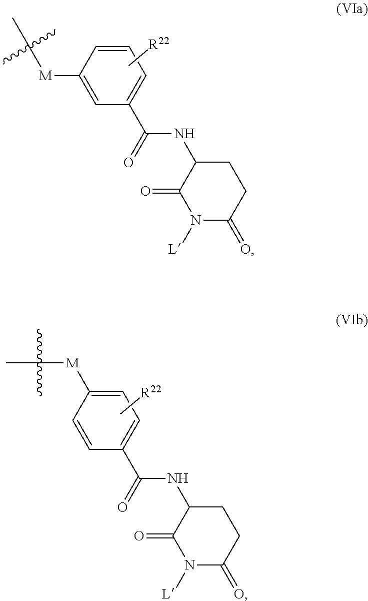

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl
   (b) Formula (II):

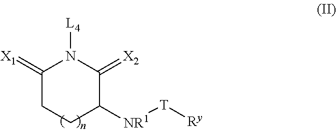

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl; n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from

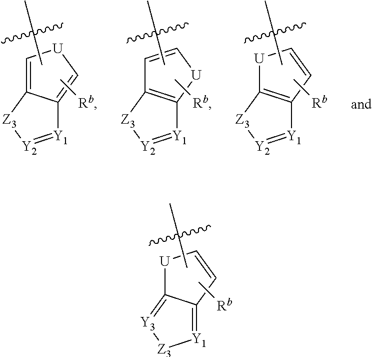

wherein [symbol] indicates attachment to T,
   Z₃ is O, S or NR³;
   U is O, S, NR^{b} or CRⁱ₂;
   each of Y₁, Y₂ and Y₃ is independently N or CR^{d};
   each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}′, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
   each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
   each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
   each R^{b} is independently alkyl, cycloalkyl, alkenyl, cycloalkenyl, aryl, heteroaryl, or benzyl;
   R²¹ is a bond connected to R¹⁸ of the linker, wherein Formula (II) contains a single R²¹;
   wherein,
   (i) when R^{y} is

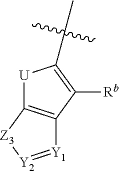

then Y₂ is CR^{d}; and
(ii) when R^{y} is

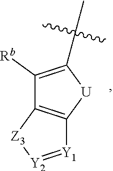

then Rⁱ in CRⁱ₂ is not hydrogen
or
   (c) Formula (III):

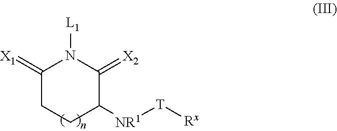

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from

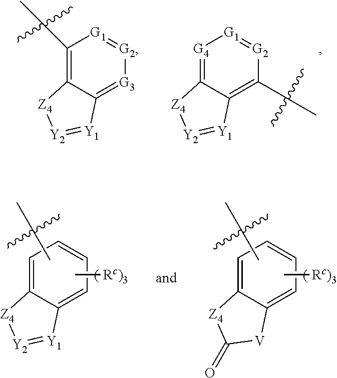

wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{C}, then C=X₁ may be replaced by CH;
and wherein:
   (i) when R^{x} is

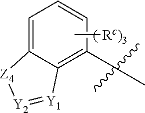

and Z₄ is NH, then L₁ is hydrogen, —CH₂C(O)OR^{b}, or —OR^{b};
(ii) when R^{x} is

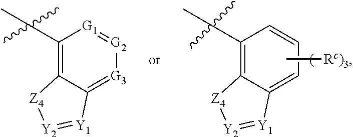

Z₄ is NR⁴, Y₁ is CR^{f}, and Y₂ is N, then R⁴ is not alkyl and at least one of R² and R is not H;
(iii) when R^{x} is

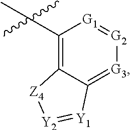

Z₄ is NR⁴, and Y₁ and Y₂ are CR^{f}, then at least one of G₁, G₂ and G₃ is N; (iv) when Z₄ is NR⁴, and Y₁ and Y₂ are CR^{f}, then R^{x} is not

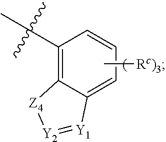

(v) when R^{x} is

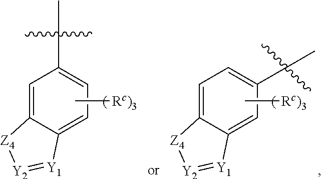

Z₄ is NR⁴, and Y₁ or Y₂ is N, then R⁴ is not alkyl;
(vi) when R^{x} is

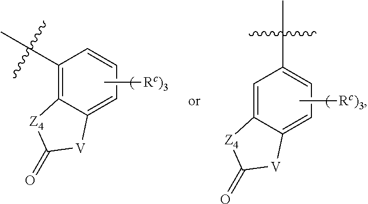

then n=1 or 2; and
(vii) when R^{x} is

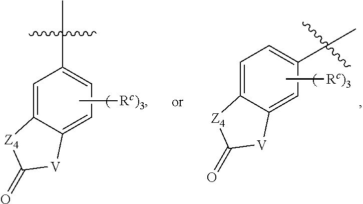

wherein [MCL-1 ligand moiety] is a compound of Formula (A1), (A2), (A3) or (A4):

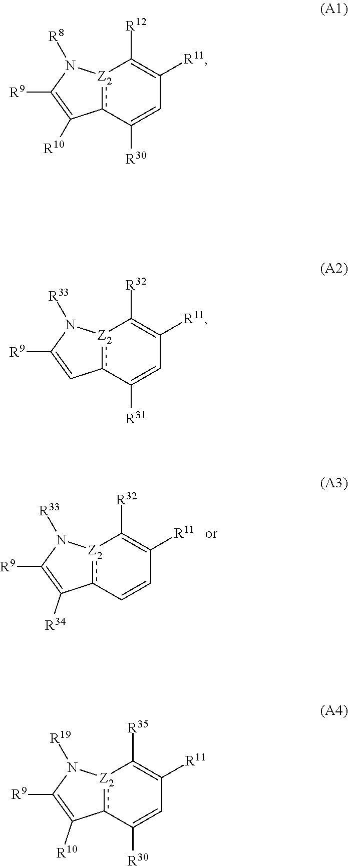

wherein
   [symbol] is a single bond or a double bond;
   each Z₂ is independently N or C, wherein when Z₂ is N, then [symbol] is a single bond; and when Z₂ is C, then [symbol] is a double bond;
   each R⁹ is independently —C(O)OH, —C(O)OC₁-C₆alkyl, —C(O)NH₂ or —P(O)(OH)₂,
   each R¹¹ is independently H, halogen or C₁-C₆ alkyl,
   R⁸ is C₁-C₆ alkyl substituted with a piperazine;
   in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹² is

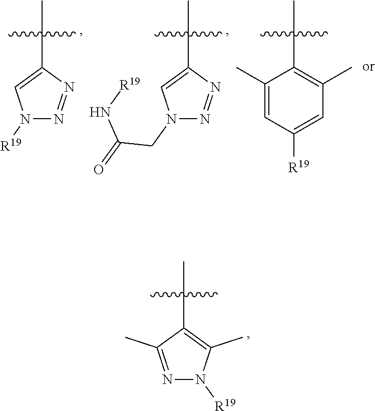

each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

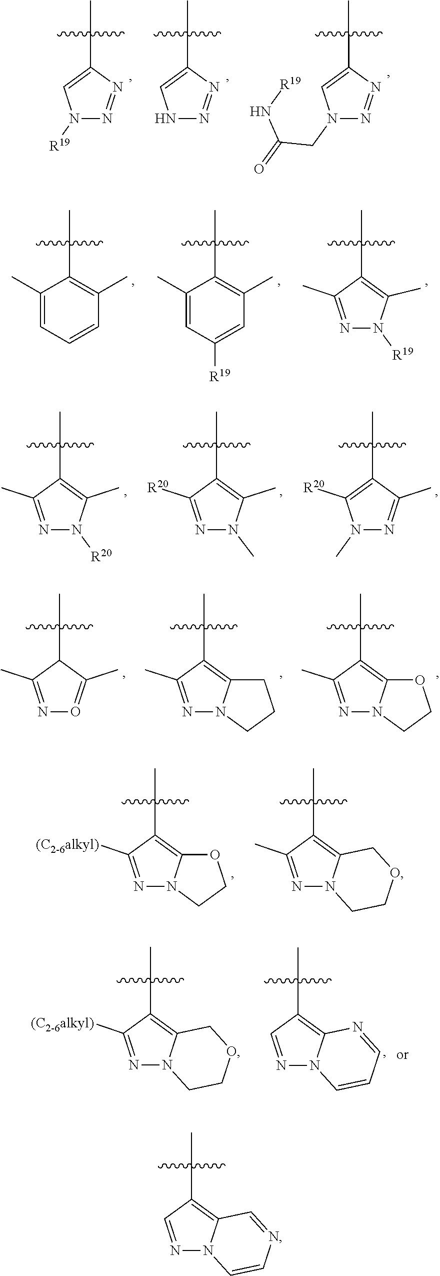

wherein R²⁰ is Me, —CH₂—OMe, —CH₂—O-bromobenzaldehyde, or

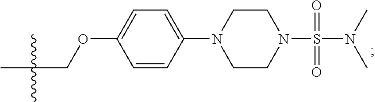 [SMILES: *COc1ccc(N2CCN(S(=O)(=O)N(C)C)CC2)cc1]

R³⁴ is —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is or

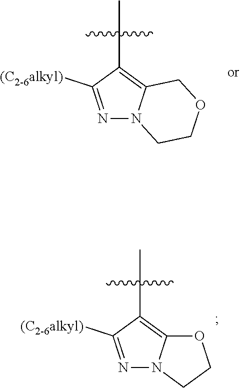

and
R¹⁹ is a bond connected to R¹⁴ of the linker, wherein each of Formula (A1), Formula (A2), Formula (A3) and Formula (A4) contains a single R¹⁹;
and wherein [linker] has the following formula

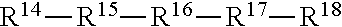

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is —C₁₋₆ alkyl, cycloalkyl, CH₂—NH—C(O)—, heterocycloalkyl, or is absent
   wherein at least one of R¹⁴-R¹⁸ is present.

43. The compound of clause 42, wherein [ligase ligand moiety] is

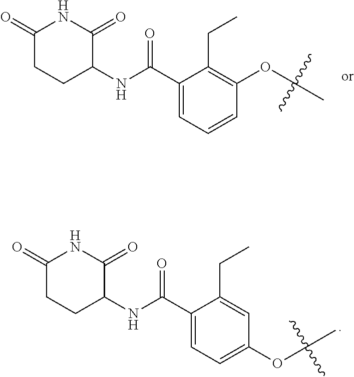 [SMILES: *Oc1cccc(C(=O)NC2CCC(=O)NC2=O)c1CC ; *Oc1ccc(C(=O)NC2CCC(=O)NC2=O)c(CC)c1]

44. The compound of clause 42, wherein [ligase ligand moiety] is Formula (III):

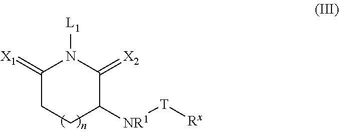

45. The compound of clause 44, wherein [ligase ligand moiety] is

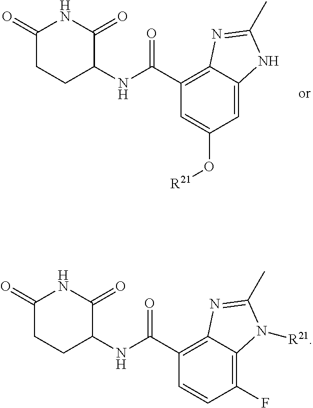

46. A compound of formula (I)

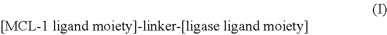

or a salt, solvate, hydrate, isomer or prodrug thereof,
wherein [ligase ligand moiety] is:

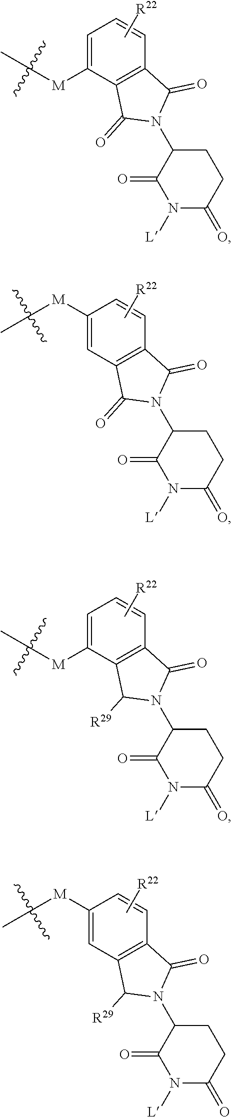

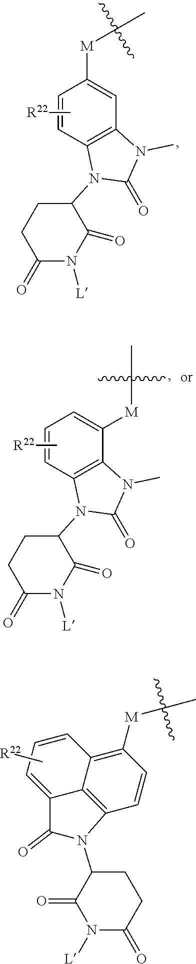

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   R²⁹ is hydrogen or Me
   L′ is hydrogen, alkyl, benzyl, acetyl or pivaloyl;
wherein [MCL-1 ligand moiety] is a compound of Formula (A):

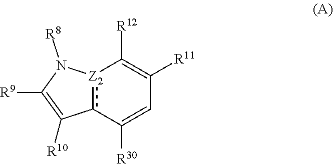

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

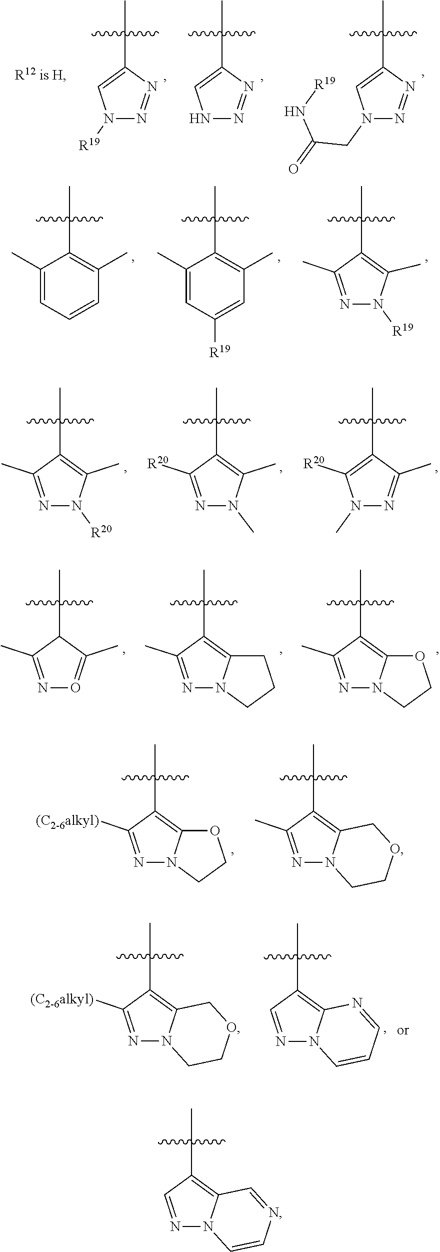

wherein R²⁰ is Me, —CH₂—OMe, —CH₂—O-bromobenzaldehyde, or

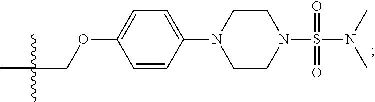 [SMILES: *COc1ccc(N2CCN(S(=O)(=O)N(C)C)CC2)cc1]

or
when R¹²

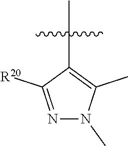

is and R¹⁰ is —O-naphthyl substituted with —O— or —S—, then R²⁰ is

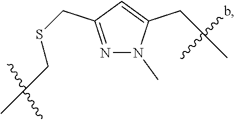

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

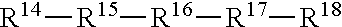

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is cycloalkyl or —CH₂—NH—C(O)—.

47. The compound of clause 46, wherein [ligase ligand moiety] is

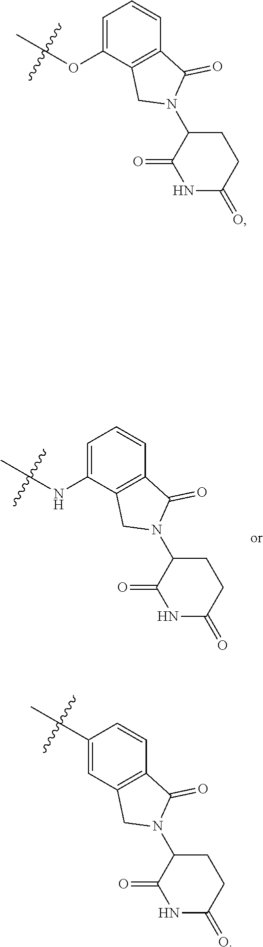 [SMILES: *Oc1cccc2c1CN(C1CCC(=O)NC1=O)C2=O ; *Nc1cccc2c1CN(C1CCC(=O)NC1=O)C2=O ; *c1ccc2c(c1)CN(C1CCC(=O)NC1=O)C2=O]

48. The compound of any one of clauses 46-47, wherein R¹⁴ is —C₁₋₆ alkyl, —C₂₋₆ alkenyl, —C₂₋₆ alkynyl, —C(O)—, —SO₂— or is absent.

49. The compound of clause 48, wherein R¹⁴ is —C₁₋₆ alkyl.

50. The compound of any one of clauses 46-49, wherein R¹⁵ is heterocycloalkyl.

51. The compound of clause 50, wherein R¹⁵ is piperazine,

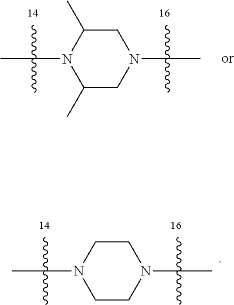

wherein [symbol] indicates attachment to R¹⁴ and [symbol] indicates attachment to R¹⁶.

52. The compound of any one of clauses 46-51, wherein R¹⁶ is —C₁₋₆ alkyl, —CH₂—C(O)— or —C(O)—.

53. The compound of any one of clauses 46-52, wherein R¹⁷ is absent.

54. The compound of any one of clauses 46-53, wherein R¹⁸ is cyclobutyl.

57. A compound of formula (I)

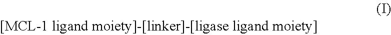

or a salt, solvate, hydrate, isomer or prodrug thereof,
wherein [ligase ligand moiety] is:
   (a) Formula (IV)

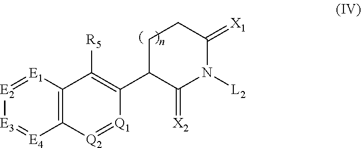

wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
and
each R^{b} is independently hydrogen, alkyl, alkenyl, aryl, heteroaryl, or benzyl; wherein R²¹ is a bond connected to R¹⁸ of the linker, and wherein Formula (IV) contains a single R²¹;
(b) Formula (VIIa), (VIIb), or (VIIc):

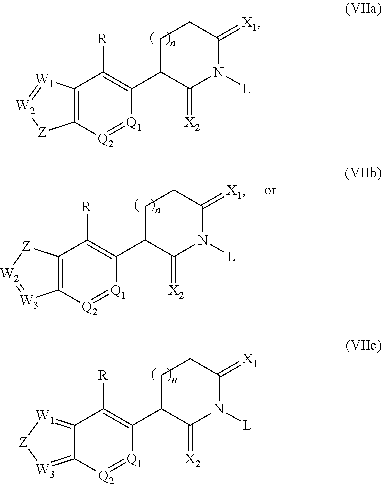

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR^{e};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII):

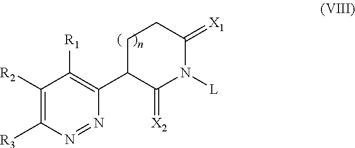

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX):

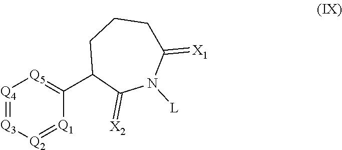

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand moiety] is a compound of Formula (A):

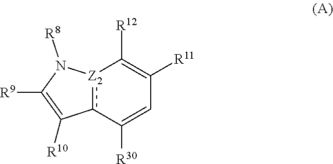

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,
   R¹² is H

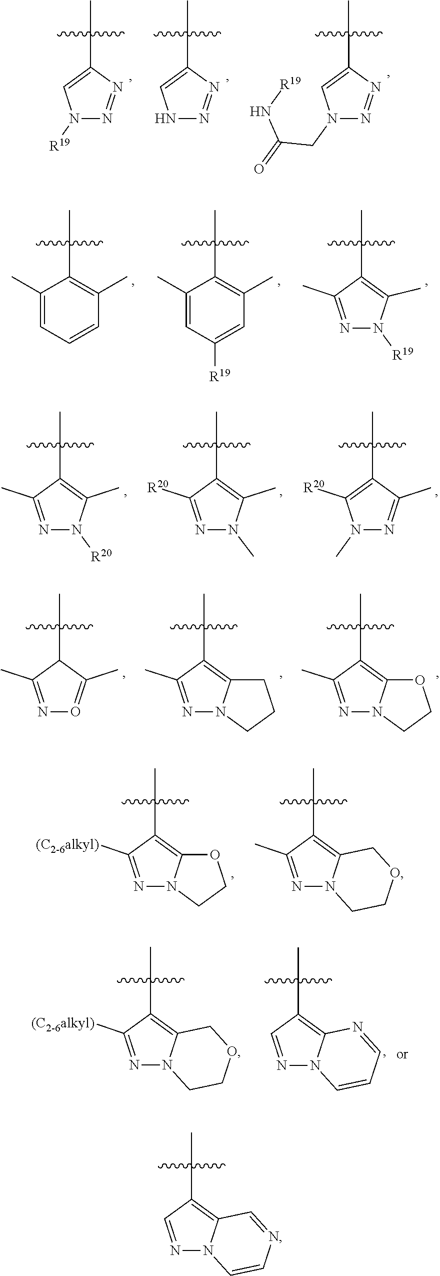

wherein R²⁰ is Me, —CH₂—Ome, —CH₂—O-bromobenzaldehyde, or

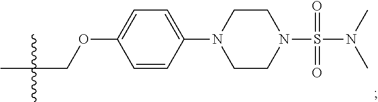 [SMILES: *COc1ccc(N2CCN(S(=O)(=O)N(C)C)CC2)cc1]

or when R¹² is

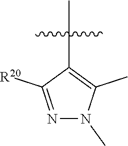

and R¹⁰ is —O-naphthyl substituted with —O— or —S—, then R²⁰ is

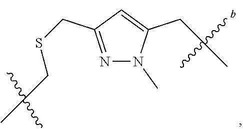 [SMILES: *CSCc1cc(C*)n(C)n1]

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] isa double bond,
   and wherein Formula (A) contains a single R¹⁹;
and wherein [linker] has the following formula

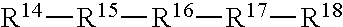

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is cycloalkyl or —CH₂—NH—C(O)—.

58. The compound of clause 57, wherein [ligase ligand moiety] is Formula (IV):

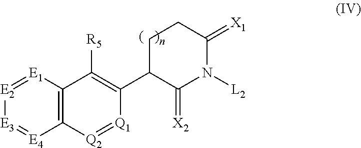

59. The compound of clause 57 or 58, wherein each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹.

60. The compound of clause 59, wherein each R^{b} is independently hydrogen, alkyl, cycloalkyl, or aryl.

61. The compound of clause 60, wherein the aryl is substituted with one or more groups selected from halogen, alkyl and O-haloalkyl, optionally wherein the halogen is Cl, the alkyl is methyl and the O-haloalkyl is O—CF₃.

62. The compound of any one of clauses 57-61, wherein one of E₁, E₂, E₃ and E₄ is N, and the remaining three of E₁, E₂, E₃ and E₄ are each CR′; optionally wherein:
(a) E₁ is N, and E₂, E₃ and E₄ are CR′;
(b) E₂ is N, and E₁, E₃ and E₄ are CR′;
(c) E₃ is N, and E₁, E₂ and E₄ are CR′; OR
(d) E₄ is N, and E₁, E₂ and E₃ are CR′.

63. The compound of any one of clauses 57-61, wherein E₁, E₂, E₃ and E₄ are each CR′, optionally wherein E₁, E₂, E₃ and E₄ are each CH.

64. The compound of clause 63, wherein three of E₁, E₂, E₃ and E₄ are CH, and one of E₁, E₂, E₃ and E₄ is C-halogen, C-alkyl, C-alkenyl, C-alkynyl, C-aryl, C-heteroaryl, C-benzyl, C-haloalkyl, C-haloalkenyl, C—NH₂, C—NHR^{b}, C—NR^{b}₂, C—NR^{b}C(O)R^{b}, C—NR^{b}C(O)OR^{b}, C—NO₂, C—CN, C—C(O)R^{b}, C—C(O)OR^{b}, C—C(O)NH₂, C—C(O)NHR^{b}, C—C(O)NR^{b}₂, C—C(O)NHCHR^{b}₂, C—CHR^{b}NHC(O)NHR^{b}, C—CHR^{b}NHC(O)C(halogen)₂R^{b}, C—OR^{b}, C—OC(O)R^{b}, C—OC(O)OR^{b}, C—OC(O)NH₂, C—OC(O)NHR^{b}, C—OC(O)NR^{b}₂, C—SR^{b}, C—S(O)₂R^{b}, C—S(O)₂OR^{b}, C—S(O)₂NH₂, C—S(O)₂NHR^{b}, C—S(O)₂NR^{b}₂, C—NHS(O)₂R^{b}, —R², —O—R²¹, —NH—R²¹, —C(O)—R²¹, —C(O)—NH—R²¹, or —CH₂—NH—C(O)—R²¹.

65. The compound of clause 64, wherein:
(a) E₂, E₃ and E₄ are each CH;
(b) E₁, E₃ and E₄ are each CH; or
(c) E₁, E₂ and E₄ are each CH.

66. The compound of any one of clauses 57-61, wherein two of E₁, E₂, E₃ and E₄ are N, and the remaining two of E₁, E₂, E₃ and E₄ are each CR′.

67. The compound of any one of clauses 57-61, wherein three of E₁, E₂, E₃ and E₄ are N, and the remaining one of E₁, E₂, E₃ and E₄ is CR′.

68. The compound of any one of clauses 57-67 wherein:
(a) Q₁ is N and Q₂ is CR;
(b) Q₁ is N and Q₂ is N; or
(c) Q₁ is CR and Q₂ is N, optionally wherein Q₁ is C—H or C-alkyl, further optionally wherein Q₁ is C—H or C-Me.

69. The compound of any one of clauses 57-68, wherein R¹⁴ is —C₁₋₆ alkyl, —C₂₋₆ alkenyl, —C₂₋₆ alkynyl, —C(O)—, —SO₂— or is absent

70. The compound of clause 69, wherein R¹⁴ is —C₁₋₆ alkyl.

71. The compound of any one of clauses 57-70, wherein R⁵ is cycloalkyl, heterocycloalkyl, aryl, heteroaryl, —C₁₋₆ alkyl-NH—, -cycloalkyl-NH— or is absent.

72. The compound of clause 71, wherein R⁵ is heterocycloalkyl or is absent.

73. The compound of clause 72, wherein R⁵ is piperazine.

74. The compound of any one of clauses 57-73, wherein R¹⁶ is —C(O)—.

75. The compound of any one of clauses 57-74, wherein R¹⁷ is absent.

76. The compound of any one of clauses 57-75, wherein R¹⁸ is —CH₂—NH—C(O)—.

77. The compound of any one of clauses 57-76, wherein [linker] is

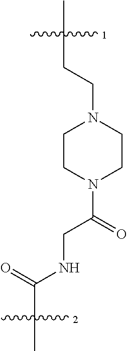 [SMILES: *CCN1CCN(C(=O)CNC(*)=O)CC1]

wherein
   [symbol] indicates attachment to [MCL-1 ligand moiety] and
   [symbol] indicates attachment to [ligase ligand moiety].

78. The compound of any one of clauses 57-77, wherein [MCL-1 ligand moiety] is:

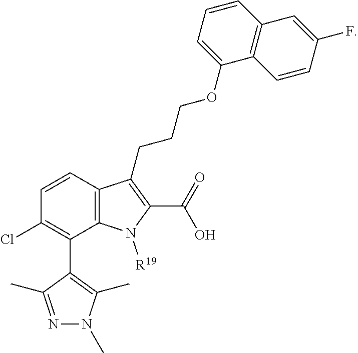

79. The compound of clause 57, wherein the compound is selected from:

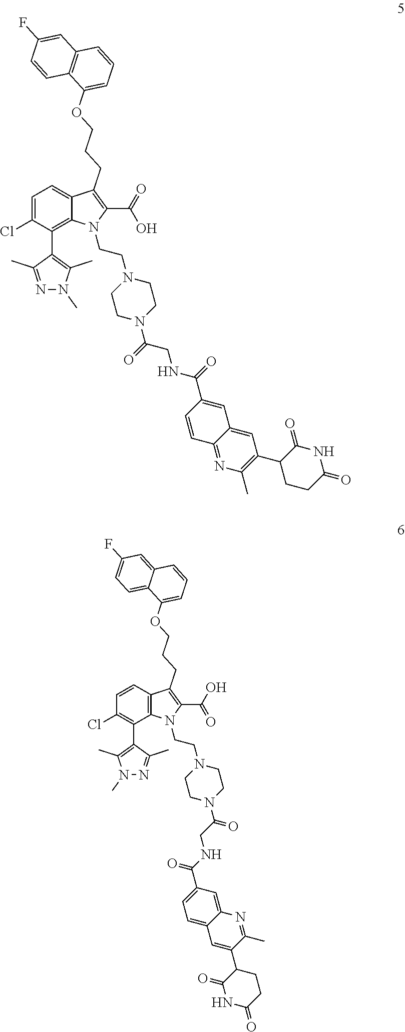

80. The compound of clause 57, wherein the compound is

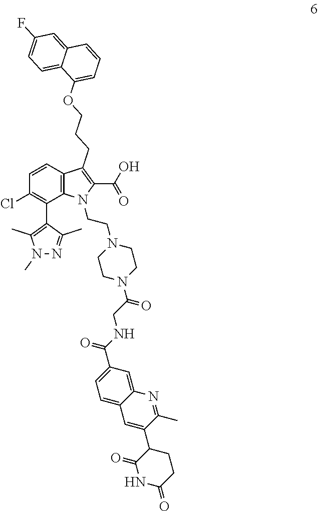

81. A compound of formula (I)

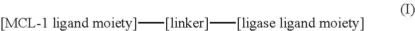

or a salt, solvate, hydrate, isomer or prodrug thereof,
wherein [ligase ligand moiety] is:
   (a) Formula (Va) or (Vb):

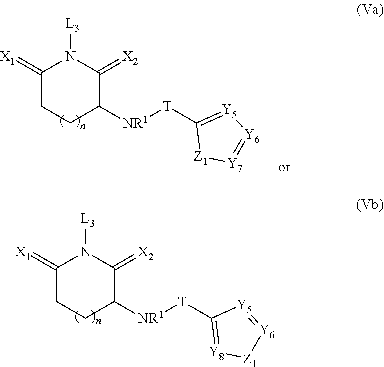

or a pharmaceutically acceptable salt or tautomer thereof,
   wherein
   each of X₁ and X₂ is independently O or S;
   Z₁ is O, S or NR⁶;
   T is is C═O or SO₂;
   R¹ is hydrogen, alkyl, cycloalkyl, alkenyl, cycloalkenyl, aryl, heteroaryl, or benzyl;
   each of Y₅, Y₆, Y₇, and Y₈ is independently N or CR⁷,
   wherein at least one of Y₅, Y₆ and Y₇ in Formula (Va) is CR⁷, and at least one of Y₅, Y₆ and Y₈ in Formula (Vb) is CR⁷;
   n is 0, 1 or 2;
   L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
   each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
   each R″″ is independently hydrogen, alkyl, cycloalkyl, alkenyl, cycloalkenyl, aryl, heteroaryl, or benzyl;
   R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
   wherein R²¹ is a bond connected to R¹⁸ of the linker, and wherein formula (Va) and formula (Vb) each contain a single R²¹;
   wherein when Z₁ is 0, then Y₆ is CR⁷ and
wherein when the compound is of Formula (Va), then
   (i) when each of Y₅, Y₆ and Y₇ is CR⁷, then at least one of R⁷ is not H;
   (ii) when Z₁ is NR⁶, then Y₆ and Y₇ are CR⁷;
   (iii) when Z₁ is S, then Y₅ is not C—Ome and Y₆ is not C—Ome;
   (iv) when Z₁ is S and Y₅ is C—NHCOMe, then Y₇ is not C—CH₂NR″″C(O)OR″″;
   (v) when Z₁ is S and Y₅ is N, then Y₅ is not C—H, C-aryl or C—C(O)OR″″; and
   (vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
   (vii) when each of Y₅, Y₆ and Y₈ is CR⁷, then at least one of R⁷ is not H;
   (viii) when Z₁ is S, then Y₅ is not C—COOH or C—NHC(O)Me, and Y₈ is not C—Br;
   (ix) when Z₁ is S and Y₆ is C—Br, then Y₈ is C—OR″″
   (x) when Z₁ is S, Y₅ is N and Y₆ is C—H or C—NH₂, then Y₈ is not C—H
   (xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
or
   (b) Formula (IIa) or (Ilb):

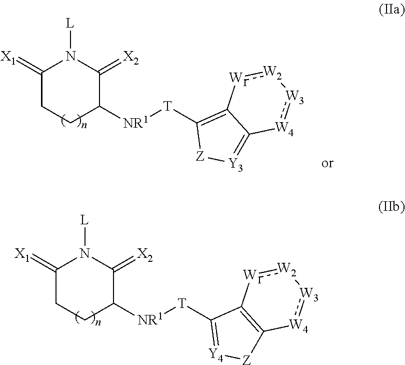

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or
   CR^{a}, wherein at least one of W₁, W₂, W₃ and W₄ is N, and
   when each [symbol] is a single bond, W₁, W₂, W₃ and W₄ are each CR^{a2} and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 ligand moiety] is a compound of Formula (A):

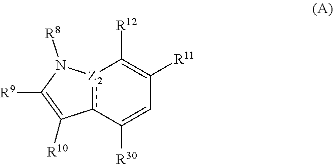

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

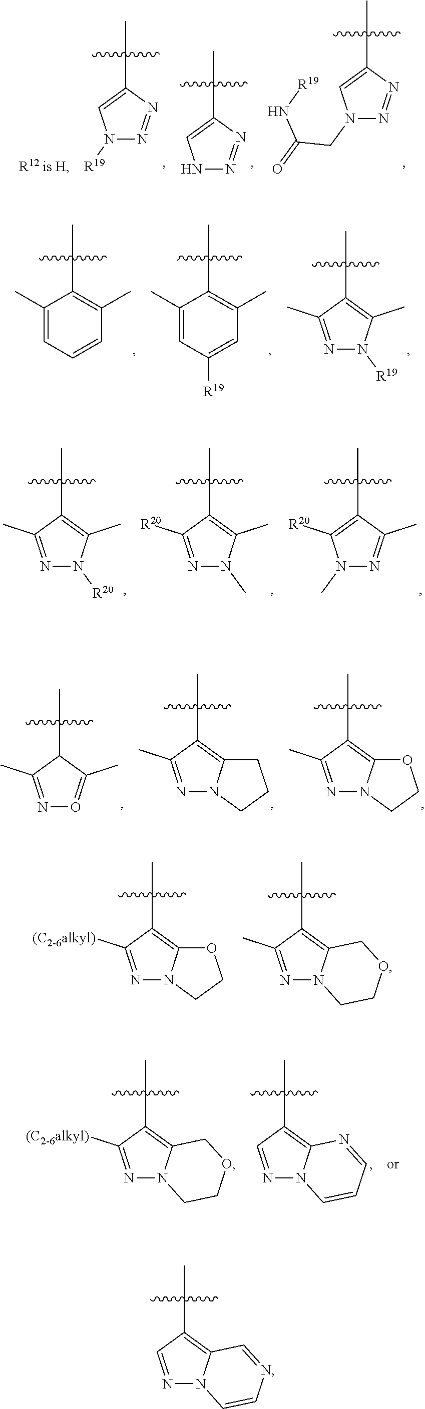

wherein R²⁰ is Me, —CH₂—OMe, —CH₂—O-bromobenzaldehyde, or

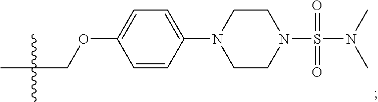 [SMILES: *COc1ccc(N2CCN(S(=O)(=O)N(C)C)CC2)cc1]

or
when R¹² is

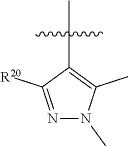

and R¹⁰ is —O-naphthyl substituted with —O— or —S—, then R²⁰ is

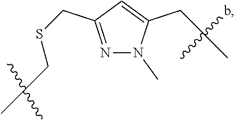

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

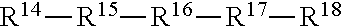

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
x is 1-10
y is 2-10
R¹⁸ is cycloalkyl or CH₂—NH—C(O)—.

82. A compound of formula (I)

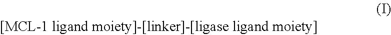

or a salt, solvate, hydrate, isomer or prodrug thereof,
wherein [ligase ligand moiety] is:
   (a) Formula (VIa) or (VIb):

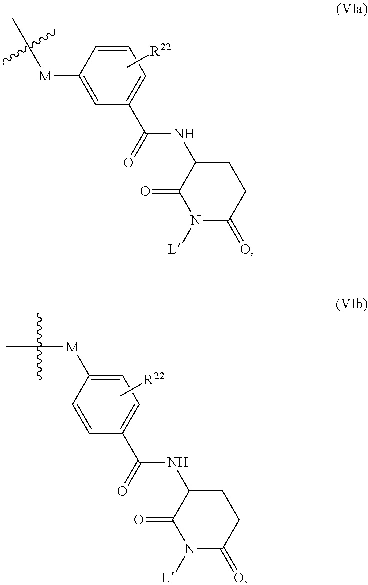

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl
   (b) Formula (II):

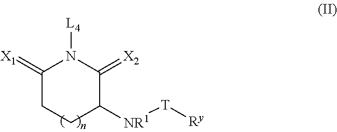

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from

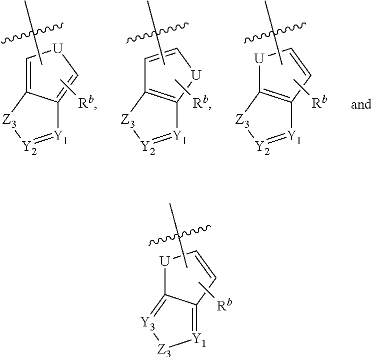

wherein [symbol] indicates attachment to T,
   Z₃ is O, S or NR³;
   U is O, S, NR^{b} or CRⁱ₂;
   each of Y₁, Y₂ and Y₃ is independently N or CR^{d};
   each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
   each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
   each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
   each R^{b} is independently alkyl, cycloalkyl, alkenyl, cycloalkenyl, aryl, heteroaryl, or benzyl;
   R²¹ is a bond connected to R¹⁸ of the linker, wherein Formula (II) contains a single R²¹;
   wherein,
   (i) when R^{y} is

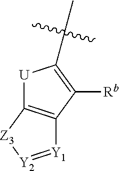

then Y₂ is CR^{d}; and
(ii) when R^{y} is

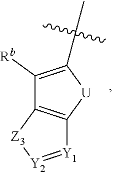

then Rⁱ in CRⁱ₂ is not hydrogen
or
   (c) Formula (III):

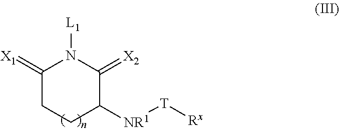

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl; n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from

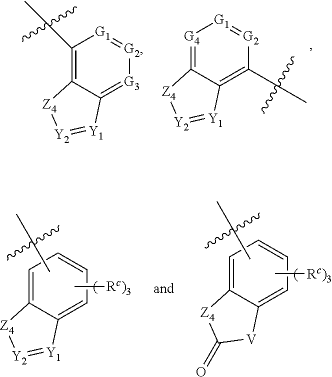

wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{c}, then C=X₁ may be replaced by CH;
and wherein:
   (i) when R^{x} is

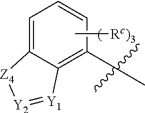

and Z₄ is NH, then L₁ is hydrogen, —CH₂C(O)OR^{b}, or —OR^{b};
(ii) when R^{x} is

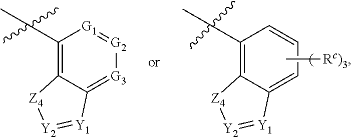

Z₄ is NR⁴, Y₁ is CR^{f}, and Y₂ is N, then R⁴ is not alkyl and at least one of R² and R is not H;
(iii) when R^{x} is

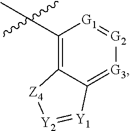

Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not

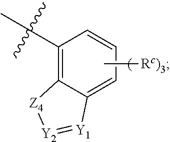

(v) when R^{x} is

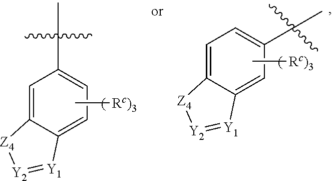

Z₄ is NR⁴, and Y₁ or Y₂ is N, then R⁴ is not alkyl;
(vi) when R^{x} is

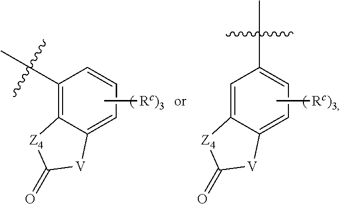

then n=1 or 2; and
(vii) when R^{x} is

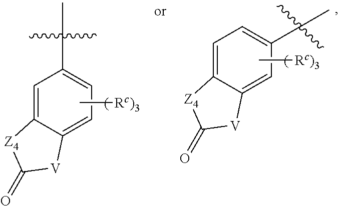

then Z₄=O or S
wherein [MCL-1 ligand moiety] is a compound of Formula (A):

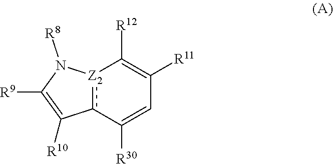

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,

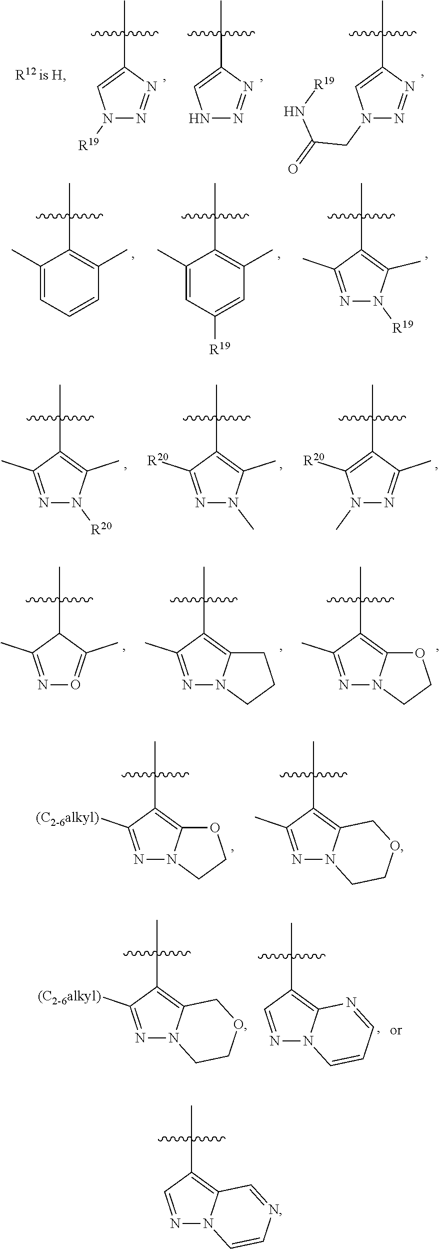

wherein R²⁰ is Me, —CH₂—OMe, —CH₂—O-bromobenzaldehyde, or

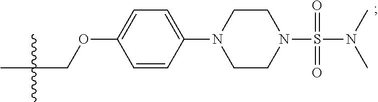 [SMILES: *COc1ccc(N2CCN(S(=O)(=O)N(C)C)CC2)cc1]

or
when R¹² is

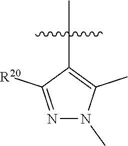

and R¹⁰ is —O-naphthyl substituted with —O— or —S—, then R²⁰ is

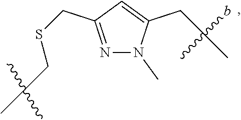

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

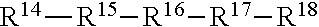

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent R⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is cycloalkyl or CH₂—NH—C(O)—.

83. The compound of any one of clauses 47-82, wherein one of R¹⁰ and R³⁰ is H and the other of R¹⁰ and R³⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

84. The compound of any one of clauses 47-83, wherein R³⁰ is H.

85. The compound of any one of clauses 47-84, wherein R¹² is H,

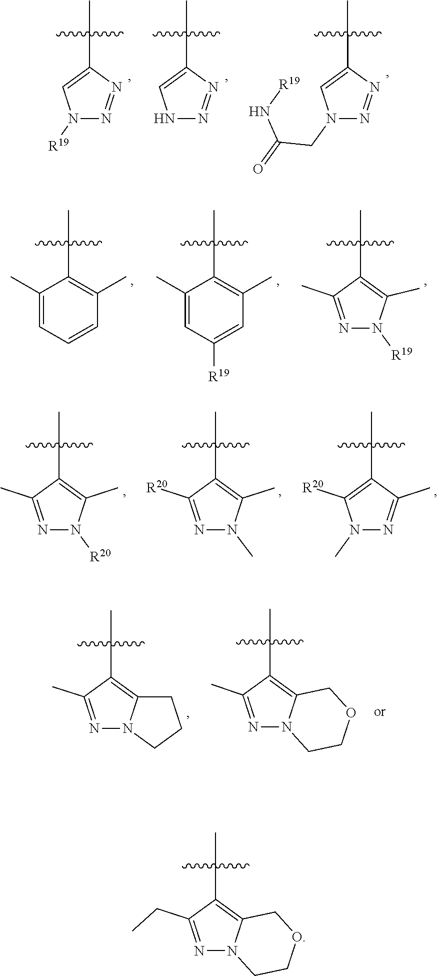

86. The compound of any one of clauses 47-85, wherein R²⁰ is Me, —CH₂—O-bromobenzaldehyde, or

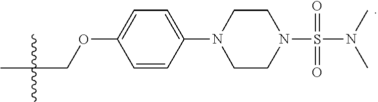 [SMILES: *COc1ccc(N2CCN(S(=O)(=O)N(C)C)CC2)cc1]

87. The compound of any one of clauses 47-86, wherein Z₂ is C and [symbol] is a double bond.

88. The compound of any one of clauses 47-87, wherein R¹¹ is hydrogen.

89. The compound of any one of clauses 47-88, wherein R¹¹ is halogen.

90. The compound of any one of clauses 47-89, wherein [MCL-1 ligand moiety] is:

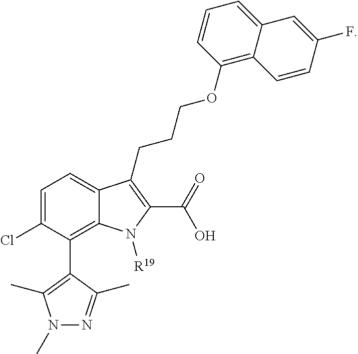

91. A compound of formula (I)

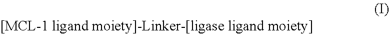

or a salt, solvate, hydrate, isomer or prodrug thereof,
wherein [ligase ligand moiety] is:

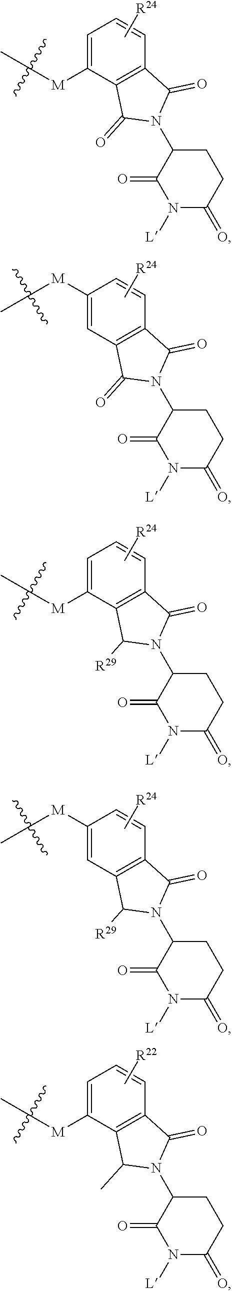

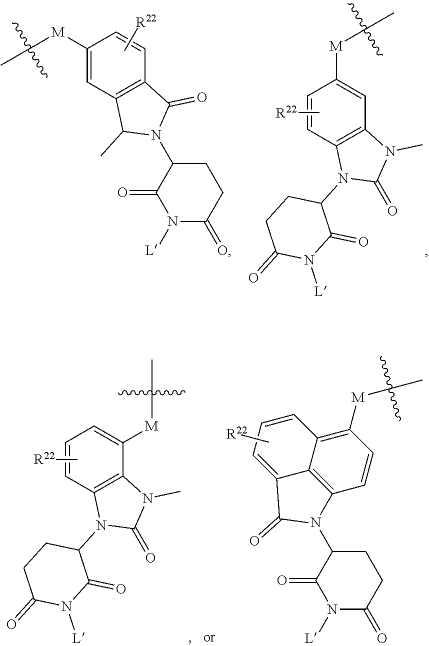

wherein
   M is O, S or NH, or is absent;
   [symbol] indicates attachment to R¹⁸ of the linker;
   R²⁴ s —OMe or heterocycloalkyl
   R²² is hydrogen, halogen, —OMe, an amino group, heterocycloalkyl, or unsubstituted C₁-C₆ alkyl;
   R²⁹ s hydrogen or Me; and
   L′ is hydrogen, alkyl, benzyl, acetyl or pivaloyl;
wherein [MCL-1 ligand moiety] is a compound of Formula (A):

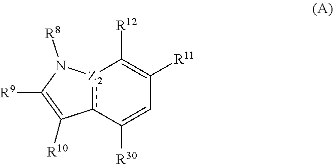

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,
   R¹² is H

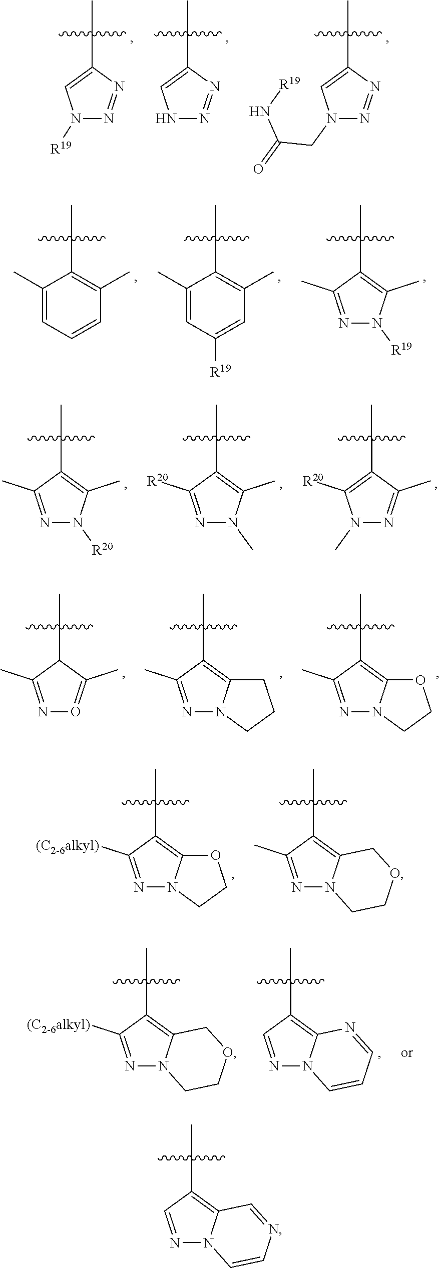

wherein R²⁰ is Me, —CH₂—OMe, —CH₂—O-bromobenzaldehyde, or

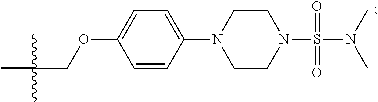 [SMILES: *COc1ccc(N2CCN(S(=O)(=O)N(C)C)CC2)cc1]

when R¹² is

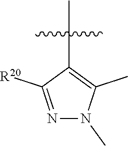

and R¹⁰ is —O-naphthyl substituted with —O— or —S—, then R²⁰ is

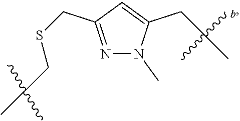 [SMILES: *CSCc1cc(C*)n(C)n1]

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

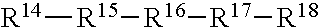

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is —C₁₋₆ alkyl, cycloalkyl, CH₂—NH—C(O)—, heterocycloalkyl, or is absent
   wherein at least one of R¹⁴-R¹⁸ is present

92. The compound of clause 91, wherein L′ is hydrogen or methyl.

93. The compound of clause 92, wherein L′ is hydrogen.

94. The compound of any one of clauses 91-93, wherein M is O or NH, or is absent.

95. The compound of clause 91, wherein [ligase ligand moiety] is

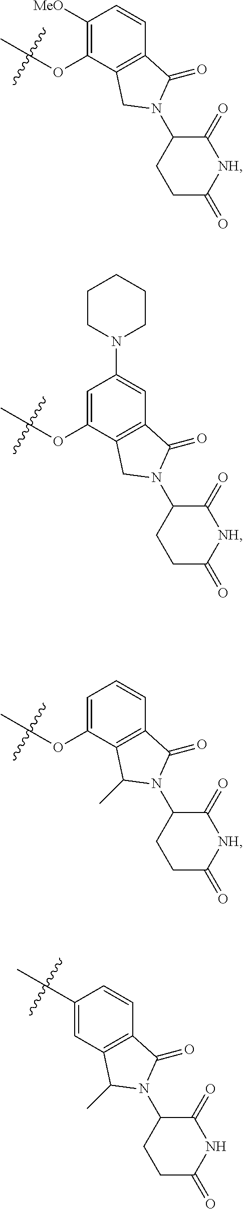 [SMILES: *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cccc2c1C(C)N(C1CCC(=O)NC1=O)C2=O ; *c1ccc2c(c1)C(C)N(C1CCC(=O)NC1=O)C2=O]

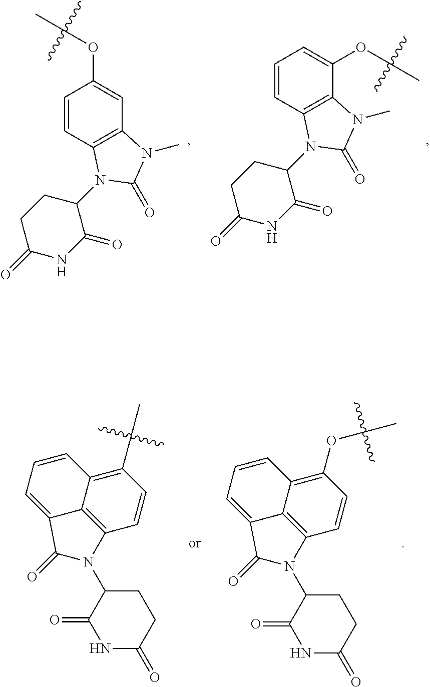 [SMILES: *Oc1ccc2c(c1)n(C)c(=O)n2C1CCC(=O)NC1=O ; *Oc1cccc2c1n(C)c(=O)n2C1CCC(=O)NC1=O ; *c1ccc2c3c(cccc13)C(=O)N2C1CCC(=O)NC1=O ; *Oc1ccc2c3c(cccc13)C(=O)N2C1CCC(=O)NC1=O]

96. The compound of any one of clauses 91-95, wherein R¹⁴ is —C₁₋₆ alkyl, —C₂₋₆ alkenyl, —C₂₋₆ alkynyl, —C(O)—, —SO₂— or is absent

97. The compound of clause 96, wherein R¹⁴ is —C₁₋₆ alkyl.

98. The compound of any one of clauses 91-97, wherein R¹⁵ is cycloalkyl, heterocycloalkyl, aryl, heteroaryl, —C₁₋₆ alkyl-NH—, -cycloalkyl-NH— or is absent.

99. The compound of clause 98, wherein R¹⁵ is heterocycloalkyl or is absent.

100. The compound of clause 99, wherein R¹⁵ is piperazine, bridged piperazine, piperazine N-oxide, piperazine cation,

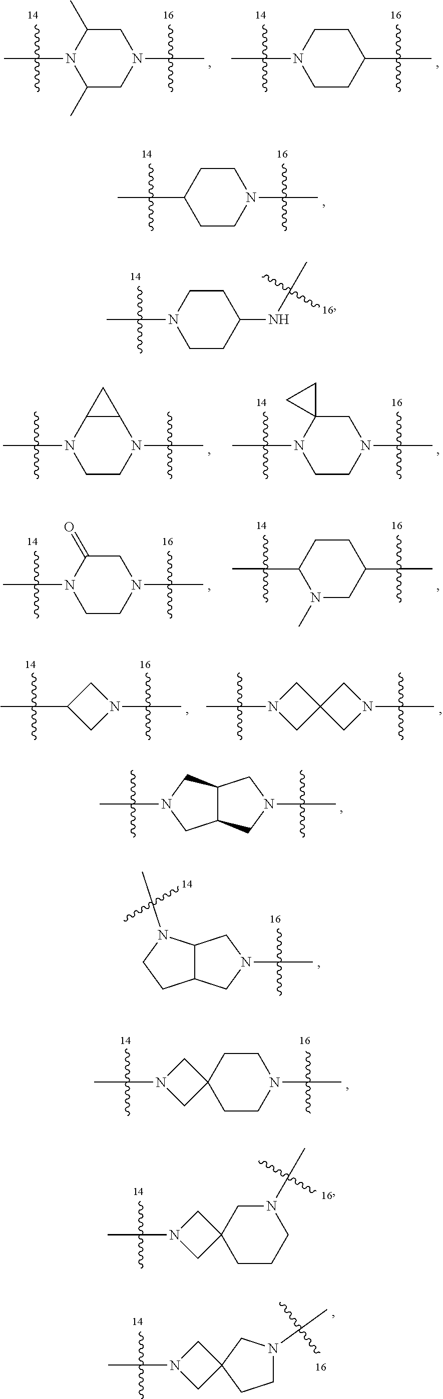

or is absent;
wherein [symbol] indicates attachment to R¹⁴ and [symbol] indicates attachment to R¹⁶.

101. The compound of clause 100, wherein R¹⁵ is piperazine,

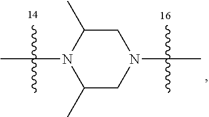 [SMILES: *N1CC(C)N(*)C(C)C1]

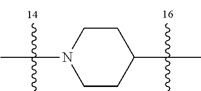 [SMILES: *C1CCN(*)CC1]

or is absent.
102. The compound of any one of clauses 91-101, wherein R¹⁶ is —C₁₋₆ alkyl, —CH₂—C(O)—NH—, —CH₂—C(O)—, —C(O)— or is absent.
103. The compound of any one of clauses 91-102, wherein R¹⁷ is absent.
104. The compound of any one of clauses 91-103, wherein R¹⁸ is —C₁₋₆ alkyl, cyclobutyl, CH₂—NH—C(O)—, piperazine or is absent.
105. The compound of any one of clauses 91-104, wherein [linker] is selected from

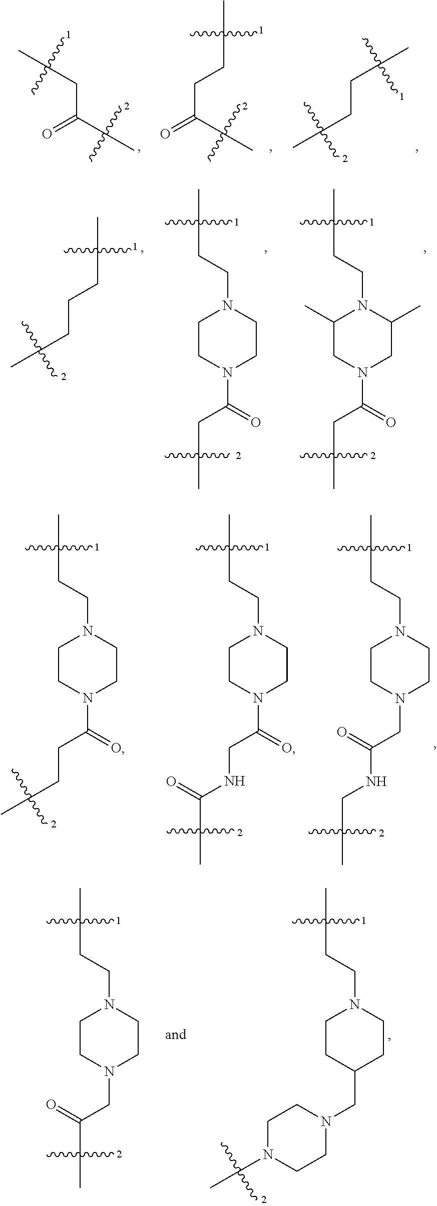

wherein
   [symbol] indicates attachment to [MCL-1 ligand moiety] and
   [symbol] indicates attachment to [ligase ligand moiety].

106. The compound of clause 105, wherein [linker] is selected from

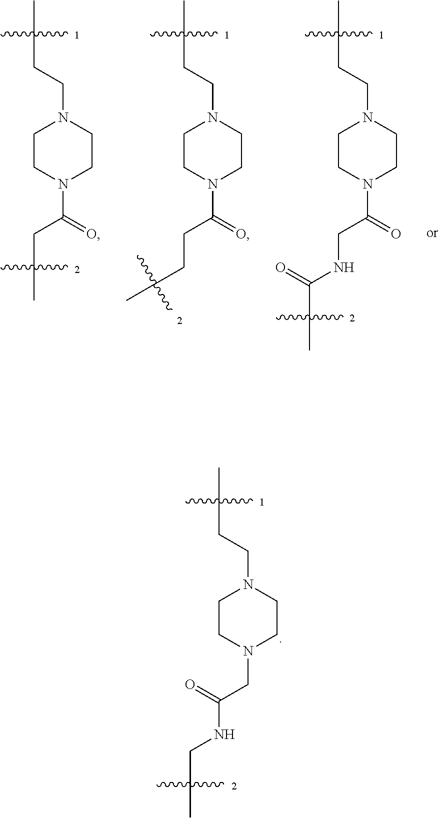 [SMILES: *CCN1CCN(C(=O)C*)CC1 ; *CCC(=O)N1CCN(CC*)CC1 ; *CCN1CCN(C(=O)CNC(*)=O)CC1 ; "or" ; *CCN1CCN(CC(=O)NC*)CC1]

107. The compound of any one of clauses 91-106, wherein [MCL-1 ligand moiety] is selected from:

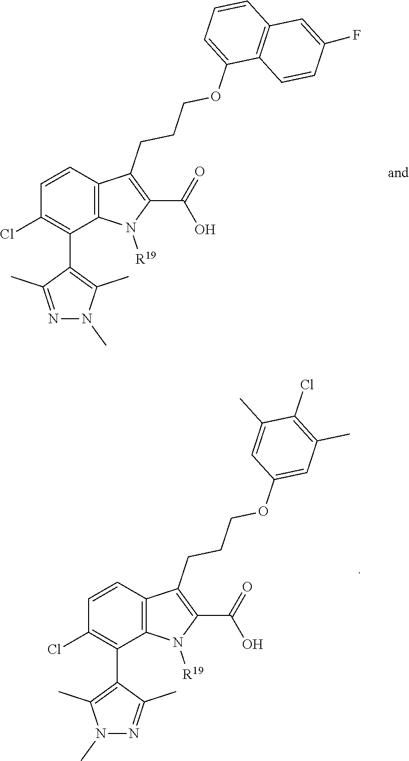

108. The compound of clause 91, which is selected from:

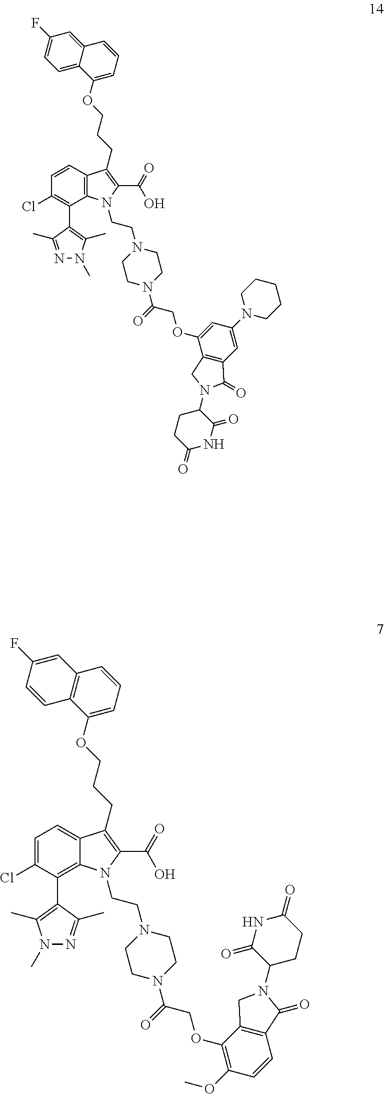 [SMILES: Cc1nn(C)c(C)c1-c1c(Cl)ccc2c(CCCOc3cccc4cc(F)ccc34)c(C(=O)O)n(CCN3CCN(C(=O)COc4cc(N5CCCCC5)cc5c4CN(C4CCC(=O)NC4=O)C5=O)CC3)c12 ; COc1ccc2c(c1OCC(=O)N1CCN(CCn3c(C(=O)O)c(CCCOc4cccc5cc(F)ccc45)c4ccc(Cl)c(-c5c(C)nn(C)c5C)c43)CC1)CN(C1CCC(=O)NC1=O)C2=O]

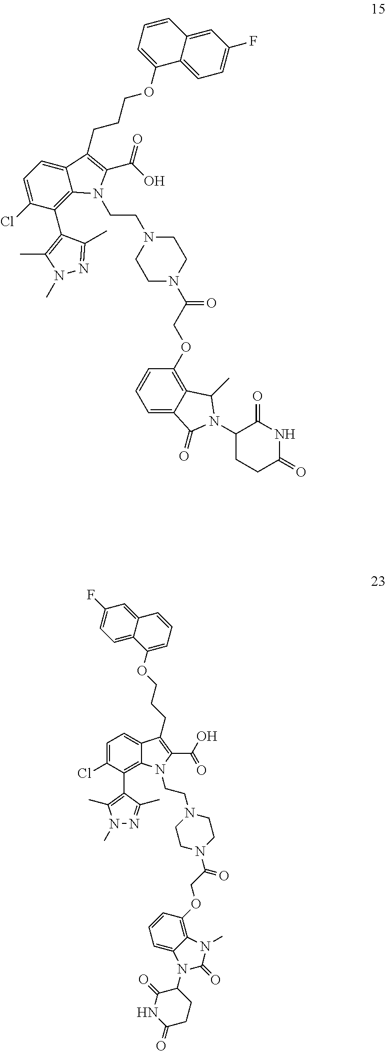 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4C(C)N(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cccc5c4n(C)c(=O)n5C4CCC(=O)NC4=O)CC3)c12]

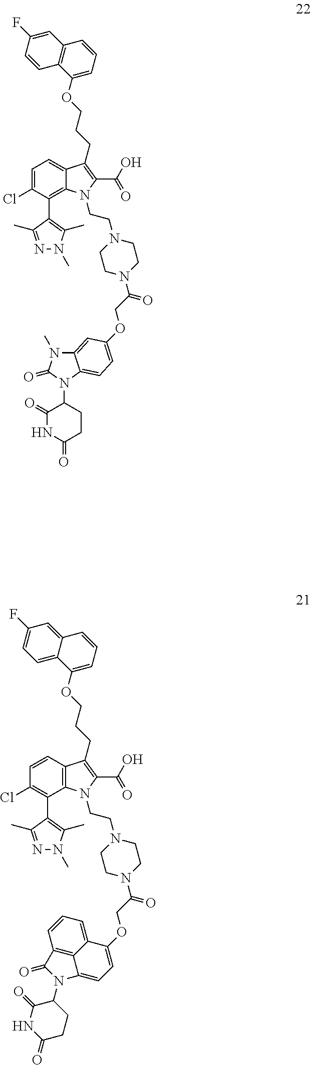 [SMILES: Cc1nn(C)c(C)c1-c1c(Cl)ccc2c(CCCOc3cccc4cc(F)ccc34)c(C(=O)O)n(CCN3CCN(C(=O)COc4ccc5c(c4)n(C)c(=O)n5C4CCC(=O)NC4=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c6c(cccc46)C(=O)N5C4CCC(=O)NC4=O)CC3)c12]

109. The compound of clause 91, which is selected from:

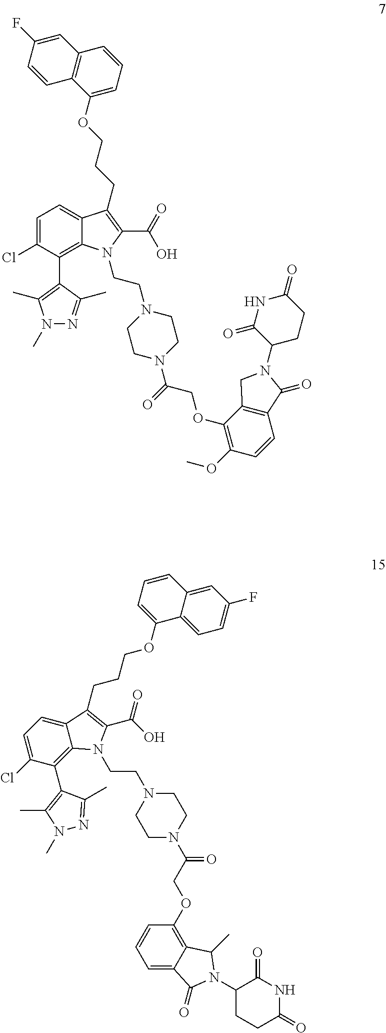 [SMILES: COc1ccc2c(c1OCC(=O)N1CCN(CCn3c(C(=O)O)c(CCCOc4cccc5cc(F)ccc45)c4ccc(Cl)c(-c5c(C)nn(C)c5C)c43)CC1)CN(C1CCC(=O)NC1=O)C2=O ; Cc1nn(C)c(C)c1-c1c(Cl)ccc2c(CCCOc3cccc4cc(F)ccc34)c(C(=O)O)n(CCN3CCN(C(=O)COc4cccc5c4C(C)N(C4CCC(=O)NC4=O)C5=O)CC3)c12]

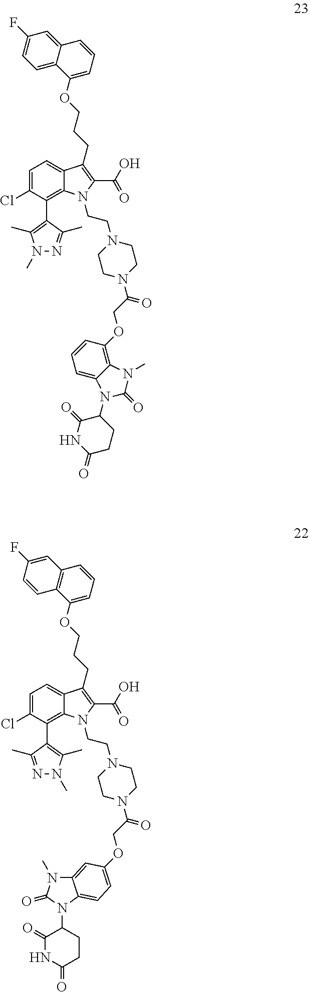 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4n(C)c(=O)n5C4CCC(=O)NC4=O)CC3)c12 ; Cc1nn(C)c(C)c1-c1c(Cl)ccc2c(CCCOc3cccc4cc(F)ccc34)c(C(=O)O)n(CCN3CCN(C(=O)COc4ccc5c(c4)n(C)c(=O)n5C4CCC(=O)NC4=O)CC3)c12]

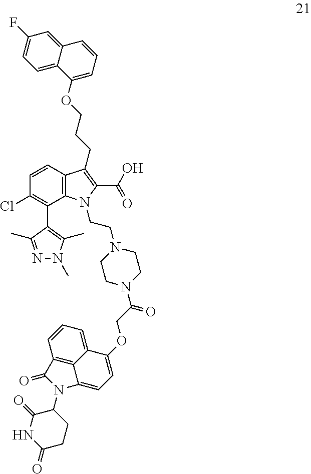 [SMILES: Cc1nn(C)c(C)c1-c1c(Cl)ccc2c(CCCOc3cccc4cc(F)ccc34)c(C(=O)O)n(CCN3CCN(C(=O)COc4ccc5c6c(cccc46)C(=O)N5C4CCC(=O)NC4=O)CC3)c12]

110. A compound of formula (I)

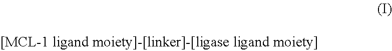

or a salt, solvate, hydrate, isomer or prodrug thereof,
wherein [ligase ligand moiety] is of Formula (VIa) or (VIb):

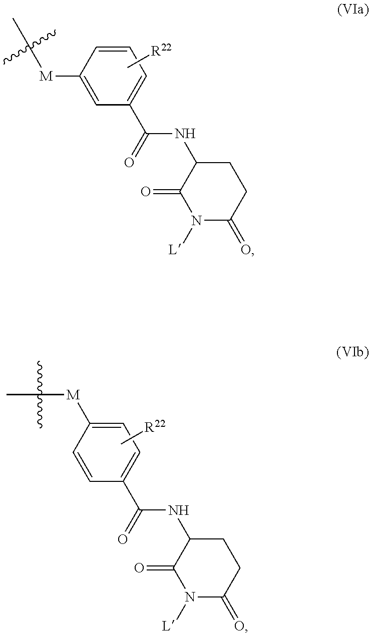

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl
wherein [MCL-1 ligand moiety] is a compound of Formula (A):

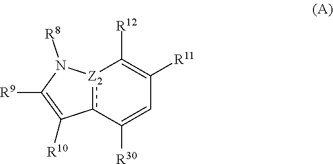

wherein
   [symbol] is a single bond or a double bond;
   R⁸ is H, R¹⁹, or C₁-C₆ alkyl optionally substituted with morpholine or a piperazine;
   R⁹ is —C(O)OH, —C(O)OC₁-C₆alkyl, —C(O)NH₂ or —P(O)(OH)₂,
   one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
   R¹¹ is H, halogen or C₁-C₆ alkyl,
   R¹² is H,

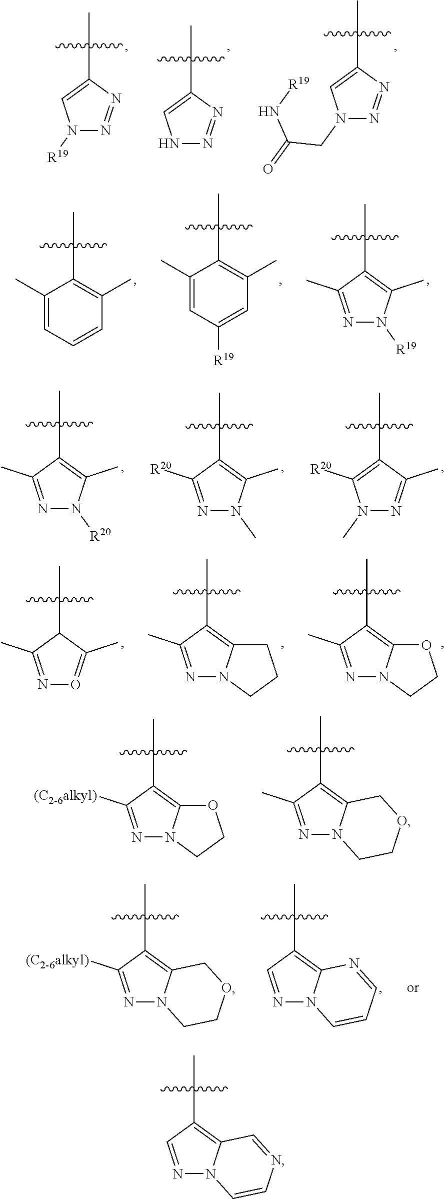

wherein R²⁰ is Me, —CH₂—OMe, —CH₂—O-bromobenzaldehyde, or

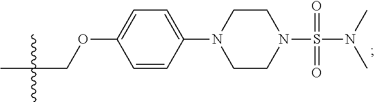 [SMILES: *COc1ccc(N2CCN(S(=O)(=O)N(C)C)CC2)cc1]

or
when R¹² is

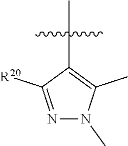

and R¹⁰ is —O-naphthyl substituted with —O— or —S—, then R²⁰ is

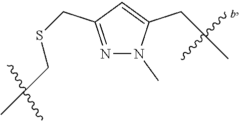 [SMILES: *CSCc1cc(C*)n(C)n1]

wherein [symbol] indicates attachment to —O— or —S— of R¹⁰;
and wherein
   R¹⁹ is a bond connected to R¹⁴ of the linker;
   Z₂ is N or C, wherein when Z₂ is N, then [symbol] is a single bond; and when Z₂ is C, then [symbol] is a double bond,
   and wherein Formula (A) contains a single R¹⁹;
and wherein [linker] has the following formula

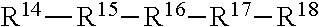

wherein
   R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, (C₂H₄—O)ₓ, (C₃H₆—O)ₓ, or is absent
   x is 1-10
   y is 2-10
   R¹⁸ is —C₁₋₆ alkyl, cycloalkyl, CH₂—NH—C(O)—, heterocycloalkyl, or is absent wherein at least one of R¹⁴-R¹⁸ is present.

111. The compound of clause 110, wherein [ligase ligand moiety] is:

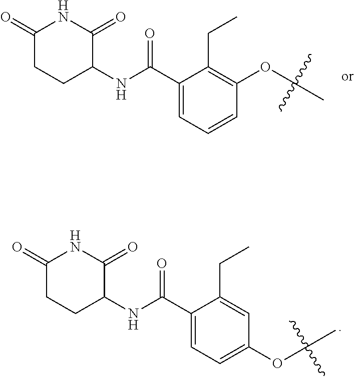 [SMILES: *Oc1cccc(C(=O)NC2CCC(=O)NC2=O)c1CC ; *Oc1ccc(C(=O)NC2CCC(=O)NC2=O)c(CC)c1]

112. The compound of any one of clauses 110-111, wherein R¹⁴ is —C₁₋₆ alkyl, —C₂₋₆ alkenyl, —C₂₋₆ alkynyl, —C(O)—, —SO₂— or is absent

113. The compound of clause 112, wherein R¹⁴ is —C₁₋₆ alkyl.

114. The compound of any one of clauses 110-113, wherein R⁵ is cycloalkyl, heterocycloalkyl, aryl, heteroaryl, —C₁₋₆ alkyl-NH—, -cycloalkyl-NH— or is absent.

115. The compound of clause 114, wherein R⁵ is heterocycloalkyl or is absent.

116. The compound of clause 115, wherein R⁵ is piperazine.

117. The compound of any one of clauses 110-116, wherein R¹⁶ is —C(O)—.

118. The compound of any one of clauses 110-117, wherein R¹⁷ is absent.

119. The compound of any one of clauses 110-118, wherein R¹⁸ is —C₁₋₆ alkyl.

120. The compound of any one of clauses 110-119, wherein [linker] is

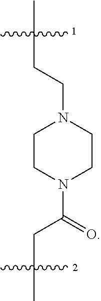 [SMILES: *CCN1CCN(C(=O)C*)CC1]

121. The compound of clause 110, wherein the compound is:

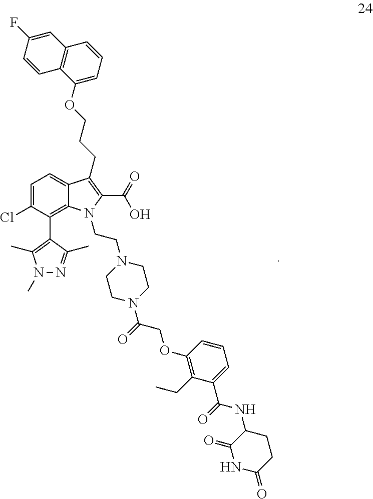 [SMILES: CCc1c(OCC(=O)N2CCN(CCn3c(C(=O)O)c(CCCOc4cccc5cc(F)ccc45)c4ccc(Cl)c(-c5c(C)nn(C)c5C)c43)CC2)cccc1C(=O)NC1CCC(=O)NC1=O]

122. The compound of any preceding clause, wherein T is C=O.

123. The compound of any preceding clause, wherein:
(a) X₁ and X₂ are O;
(b) X₁ is O and X₂ is S;
(c) X₁ is S and X₂ is O; or
(d) X₁ and X₂ are S.

124. The compound of any preceding clause, wherein n is 1.

125. The compound of any preceding clause, wherein unless otherwise specified each alkyl, alkenyl, alkynyl, aryl, heteroaryl and benzyl is unsubstituted.

126. A compound selected from:

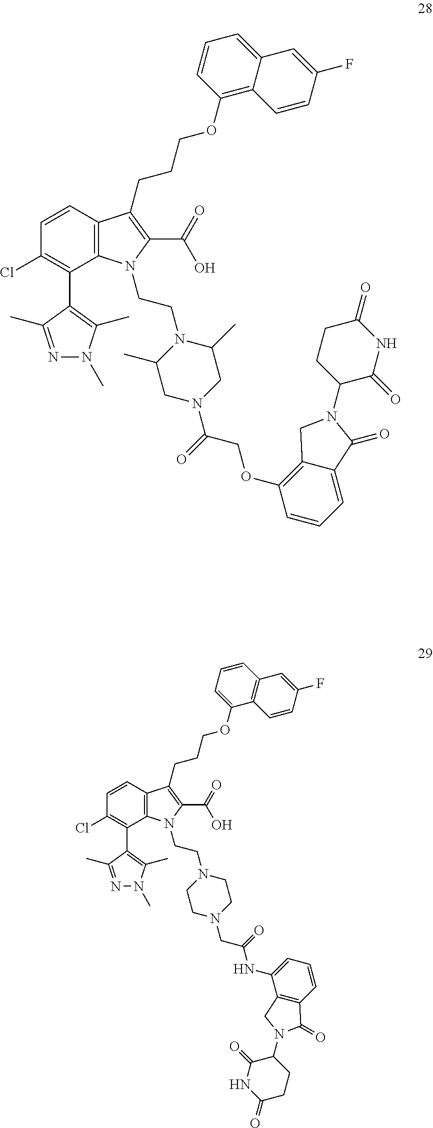 [SMILES: Cc1nn(C)c(C)c1-c1c(Cl)ccc2c(CCCOc3cccc4cc(F)ccc34)c(C(=O)O)n(CCN3C(C)CN(C(=O)COc4cccc5c4CN(C4CCC(=O)NC4=O)C5=O)CC3C)c12 ; Cc1nn(C)c(C)c1-c1c(Cl)ccc2c(CCCOc3cccc4cc(F)ccc34)c(C(=O)O)n(CCN3CCN(CC(=O)Nc4cccc5c4CN(C4CCC(=O)NC4=O)C5=O)CC3)c12]

127. A compound selected from:

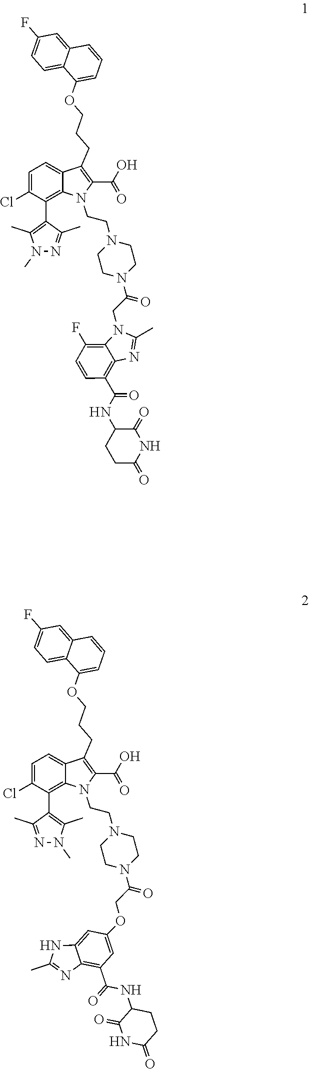

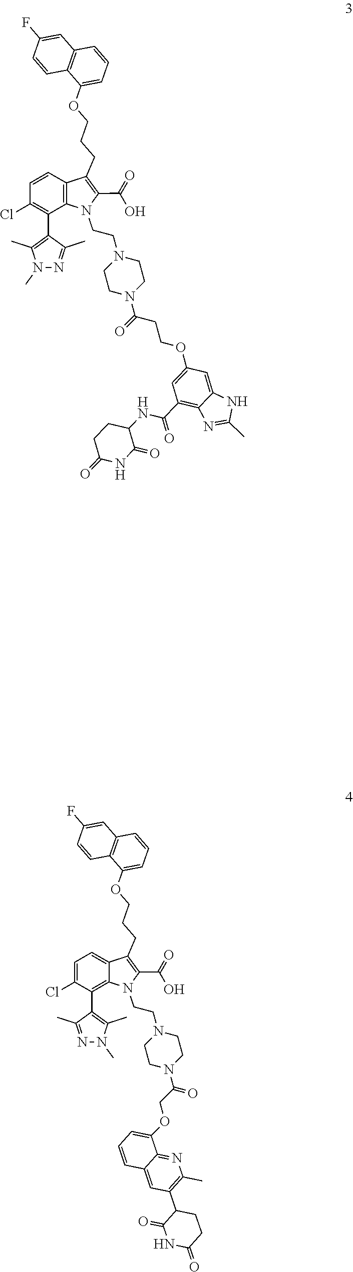

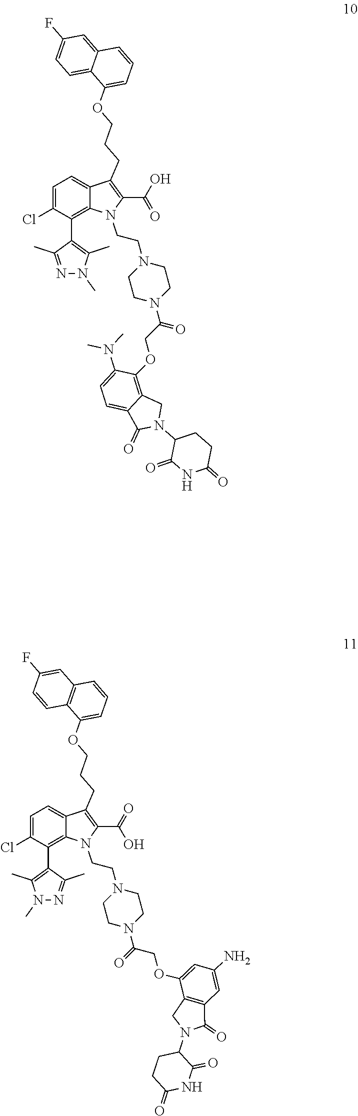 [SMILES: Cc1nn(C)c(C)c1-c1c(Cl)ccc2c(CCCOc3cccc4cc(F)ccc34)c(C(=O)O)n(CCN3CCN(C(=O)COc4c(N(C)C)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cc(N)cc5c4CN(C4CCC(=O)NC4=O)C5=O)CC3)c12]

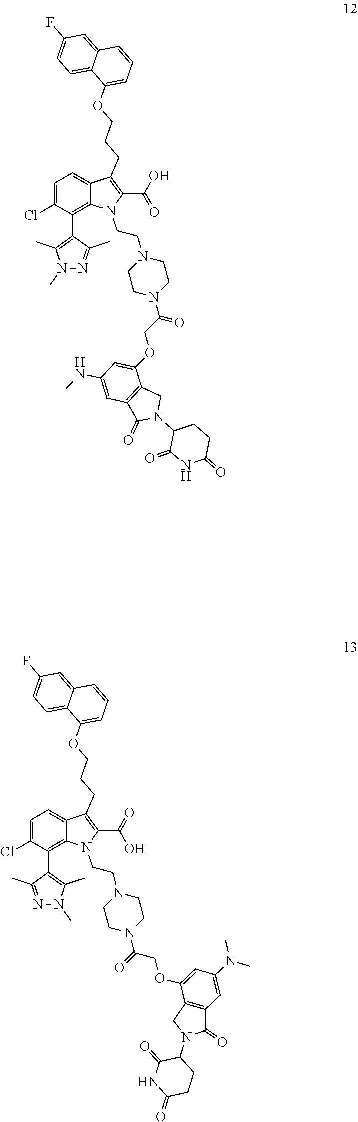

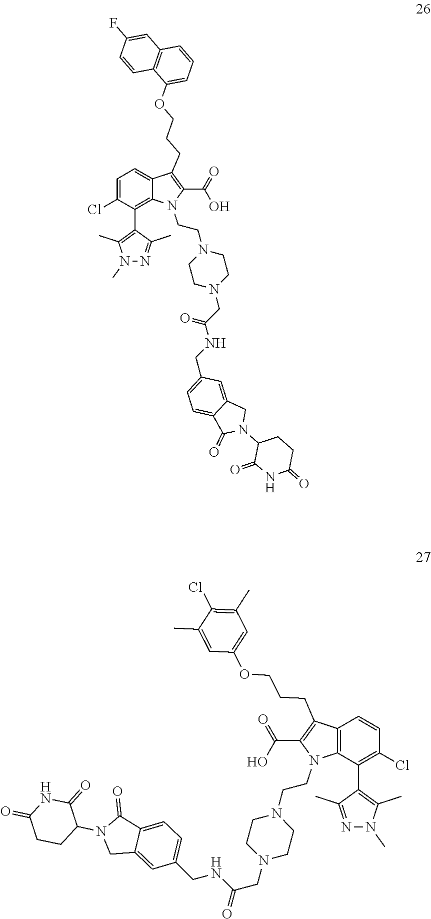 [SMILES: Cc1nn(C)c(C)c1-c1c(Cl)ccc2c(CCCOc3cccc4cc(F)ccc34)c(C(=O)O)n(CCN3CCN(CC(=O)NCc4ccc5c(c4)CN(C4CCC(=O)NC4=O)C5=O)CC3)c12 ; Cc1cc(OCCCc2c(C(=O)O)n(CCN3CCN(CC(=O)NCc4ccc5c(c4)CN(C4CCC(=O)NC4=O)C5=O)CC3)c3c(-c4c(C)nn(C)c4C)c(Cl)ccc23)cc(C)c1Cl]

128. A compound of clause 127, selected from:

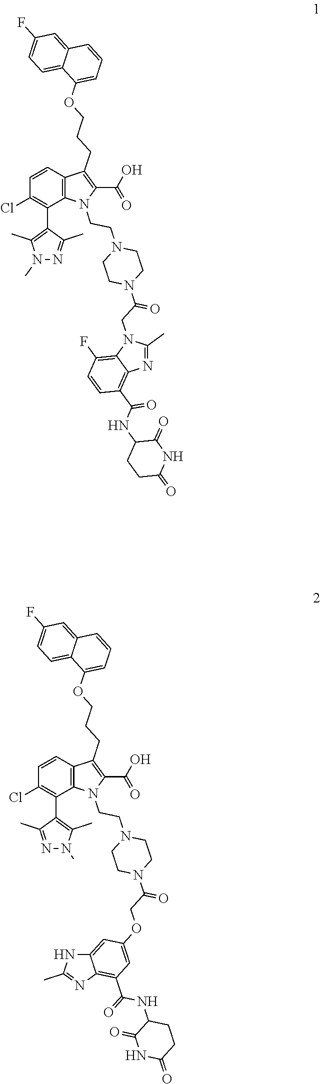 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)ccc(F)c54)CC3)c12 ; Cc1nc2c(C(=O)NC3CCC(=O)NC3=O)cc(OCC(=O)N3CCN(CCn4c(C(=O)O)c(CCCOc5cccc6cc(F)ccc56)c5ccc(Cl)c(-c6c(C)nn(C)c6C)c54)CC3)cc2[nH]1]

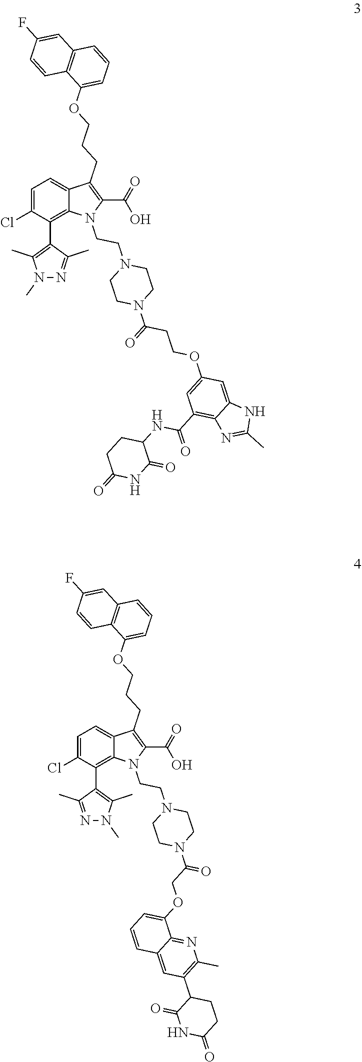

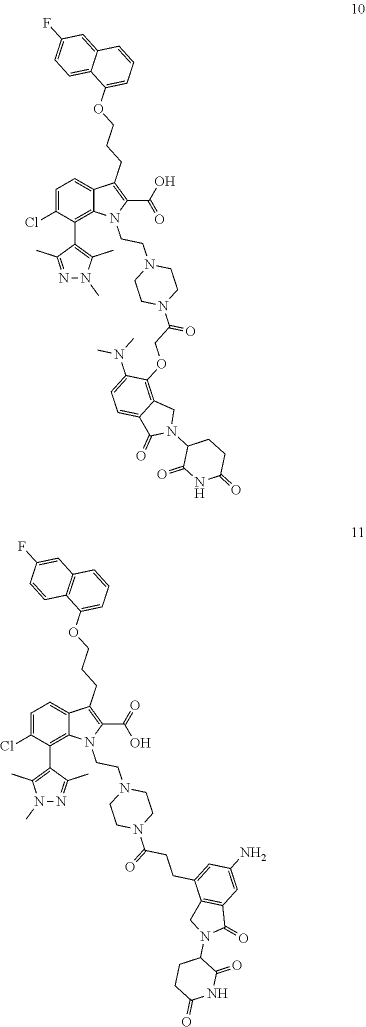 [SMILES: Cc1nn(C)c(C)c1-c1c(Cl)ccc2c(CCCOc3cccc4cc(F)ccc34)c(C(=O)O)n(CCN3CCN(C(=O)COc4c(N(C)C)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)CCc4cc(N)cc5c4CN(C4CCC(=O)NC4=O)C5=O)CC3)c12]

1. A compound of formula (I) 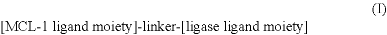
or a salt, solvate, hydrate, isomer or prodrug thereof,
wherein [ligase ligand moiety] is: 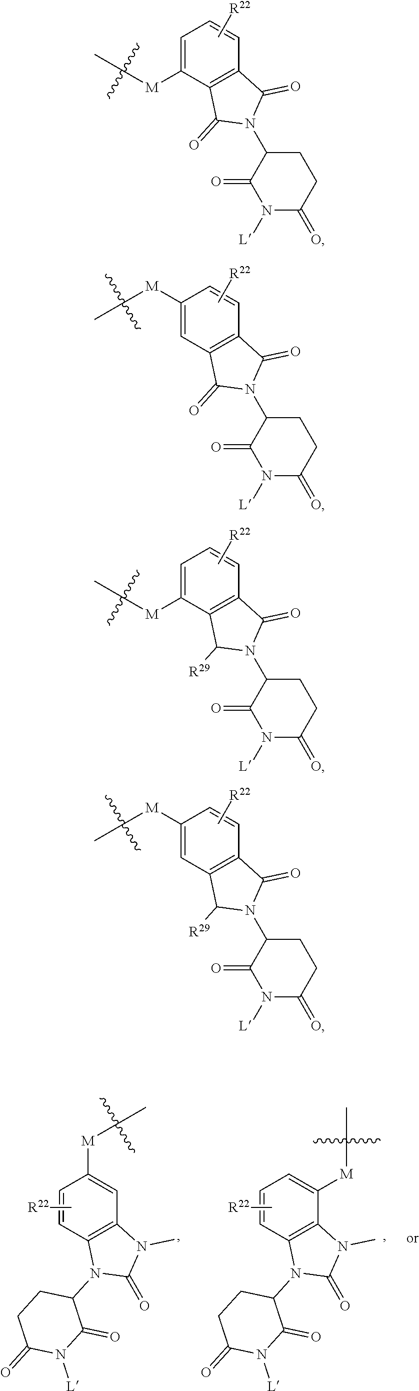 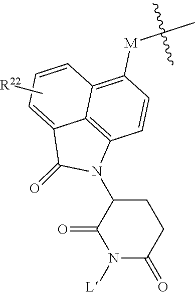
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl; and
R²⁹ is hydrogen or Me
L′ is hydrogen, alkyl, benzyl, acetyl or pivaloyl;
wherein [MCL-1 ligand moiety] is a compound of Formula (A1), (A2), (A3) or (A4): 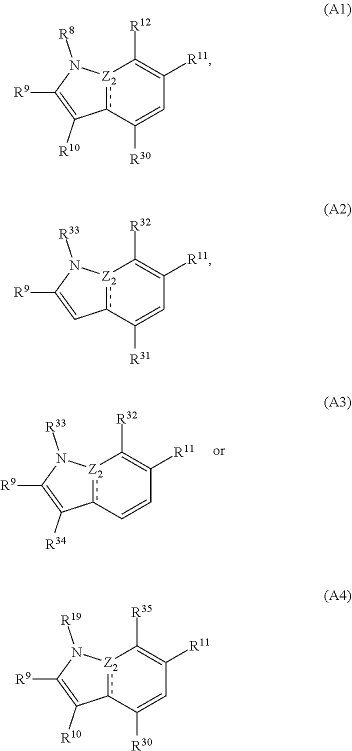
wherein [symbol]
is a single bond or a double bond;
each Z₂ is independently N or C, wherein when Z₂ is N, then [symbol] is a single bond; and when Z₂ is C, then [symbol] is a double bond;
each R⁹ is independently —C(O)OH, —C(O)OC₁-C₆alkyl, —C(O)NH₂ or P(O)(OH)₂,
each R¹¹ is independently H, halogen or C₁-C₆ alkyl,
R⁸ is C₁-C₅ alkyl substituted with a piperazine;
in each of Formula (A1) and Formula (A4), one of R¹⁰ and R³⁰ is H and the other of R¹⁰ and R³⁰ is —C₂₋₆ alkyl-O—R¹³, —O—C₂₋₆ alkyl-R¹³ or —C₂₋₆ alkyl-NMe-R¹³ wherein R¹³ is phenyl, naphthyl or tetraline,
wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 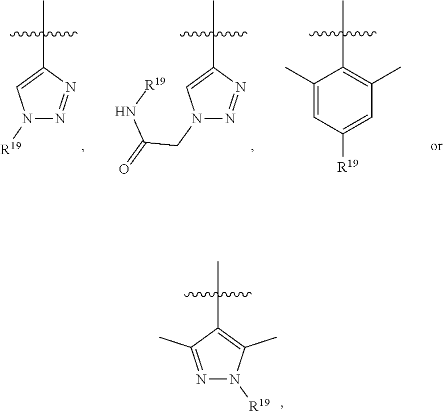
each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 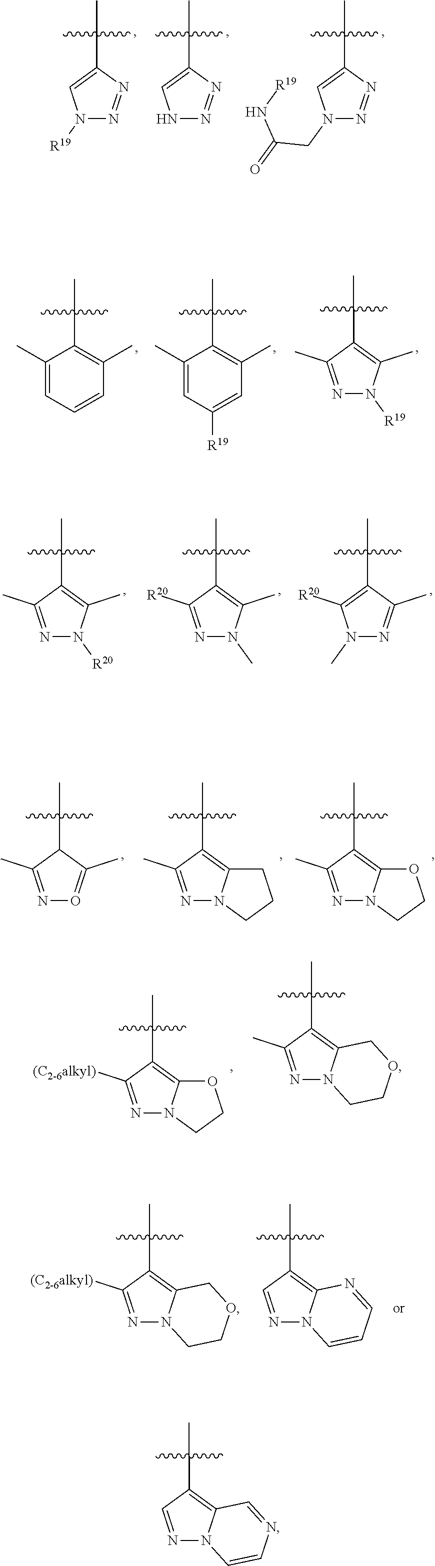
wherein R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is or 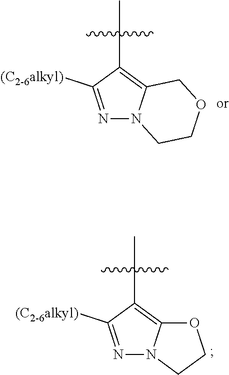
and
R¹⁹ is a bond connected to R¹⁴ of the linker, wherein each of Formula (A1), Formula (A2), Formula (A3) and Formula (A4) contains a single R¹⁹;
and wherein [linker] has the following formula 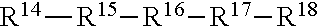
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—C₁₋₆alkyl-, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl, —C₁₋₆ alkyl-C(O)—, cycloalkyl, CH₂—NH—C(O)—, heterocycloalkyl, or is absent
wherein at least one of R¹⁴-R¹⁸ is present.

2. The compound of claim 1, wherein [ligase ligand moiety] is:

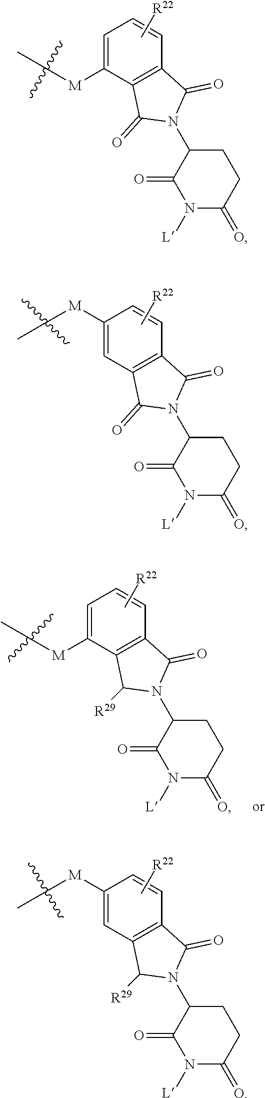

3. The compound of claim 1 or 2, wherein R²⁹ is Me.

4. The compound of claim 1 or 2, wherein R²⁹ is hydrogen.

5. The compound of claim 1, wherein [ligase ligand moiety] is:

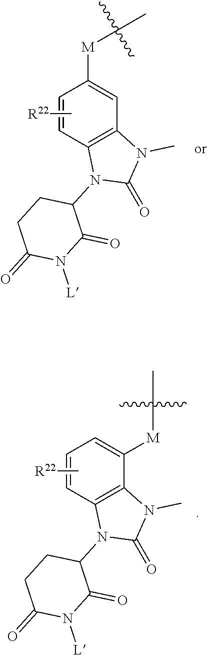

6. The compound of claim 1, wherein [ligase ligand moiety] is:

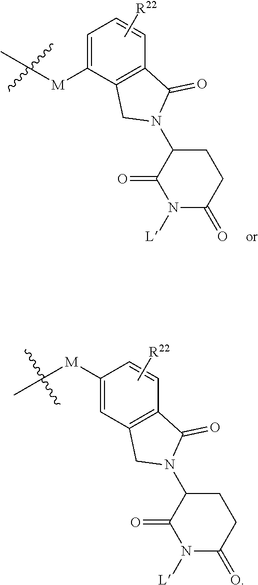

7. The compound of any preceding claim, wherein R²² is hydrogen, halogen, —OMe, —NH₂, —NHMe, —NMe₂, or piperidine; optionally wherein R²² is hydrogen, —OMe, —NH₂, —NHMe, —NMe₂, or piperidine.

8. The compound of claim 7, wherein R²² is hydrogen.

9. The compound of any preceding claim, wherein L′ is hydrogen.

10. The compound of any preceding claim, wherein M is O or NH, or is absent.

11. The compound of claim 1, wherein [ligase ligand moiety] is

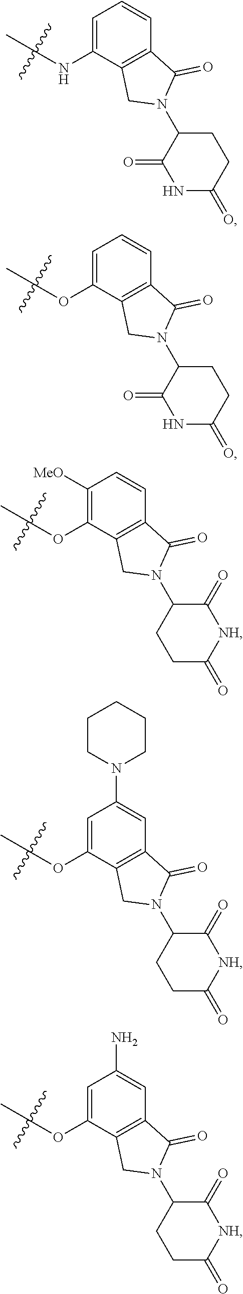 [SMILES: *Nc1cccc2c1CN(C1CCC(=O)NC1=O)C2=O ; *Oc1cccc2c1CN(C1CCC(=O)NC1=O)C2=O ; *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cc(N)cc2c1CN(C1CCC(=O)NC1=O)C2=O]

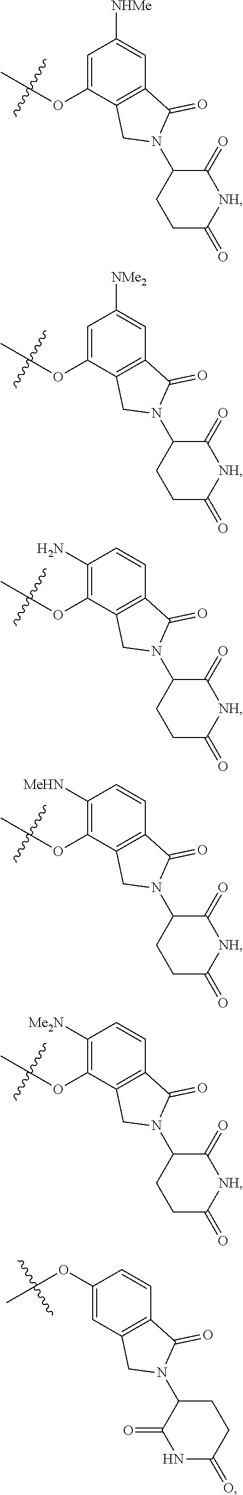 [SMILES: *Oc1cc(NC)cc2c1CN(C1CCC(=O)NC1=O)C2=O ; *Oc1cc(N(C)C)cc2c1CN(C1CCC(=O)NC1=O)C2=O ; *Oc1c(N)ccc2c1CN(C1CCC(=O)NC1=O)C2=O ; *Oc1c(NC)ccc2c1CN(C1CCC(=O)NC1=O)C2=O ; *Oc1c(N(C)C)ccc2c1CN(C1CCC(=O)NC1=O)C2=O ; *Oc1ccc2c(c1)CN(C1CCC(=O)NC1=O)C2=O]

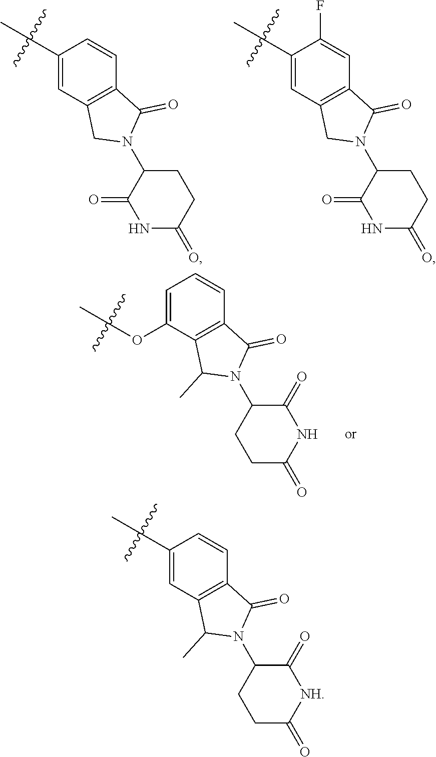

12. The compound of claim 1, wherein [ligase ligand moiety] is

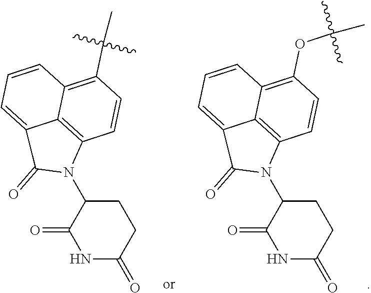 [SMILES: *c1ccc2c3c(cccc13)C(=O)N2C1CCC(=O)NC1=O ; *Oc1ccc2c3c(cccc13)C(=O)N2C1CCC(=O)NC1=O]

13. The compound of claim 1, wherein [ligase ligand moiety] is:

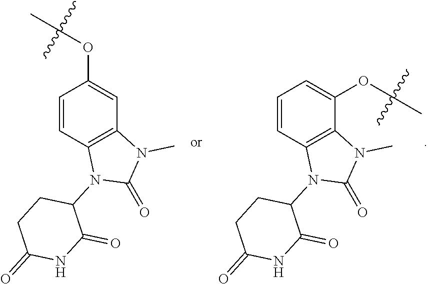 [SMILES: *Oc1ccc2c(c1)n(C)c(=O)n2C1CCC(=O)NC1=O ; *Oc1cccc2c1n(C)c(=O)n2C1CCC(=O)NC1=O]

14. The compound of any preceding claim, wherein:
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(C₂H₄—O)ₓ, —(C₃H₆—O)ₓ, or is absent
R¹⁸ is —C₁₋₆ alkyl, cycloalkyl, —CH₂—NH—C(O)—, heterocycloalkyl, or is absent.

15. The compound of any preceding claim, wherein R¹⁴ is —C₁₋₆alkyl, —C₂₋₆alkenyl, —C₂₋₆alkynyl, —C(O)—, —SO₂— or is absent.

16. The compound of claim 15, wherein R¹⁴ is —C₁₋₆ alkyl.

17. The compound of any preceding claim, wherein R¹⁵ is cycloalkyl, heterocycloalkyl, aryl, heteroaryl, —C₁₋₆ alkyl-NH—, -cycloalkyl-NH— or is absent.

18. The compound of claim 17, wherein R¹⁵ is heterocycloalkyl or is absent.

19. The compound of claim 18, wherein R¹⁵ is piperazine, bridged piperazine, piperazine N-oxide, piperazine cation, 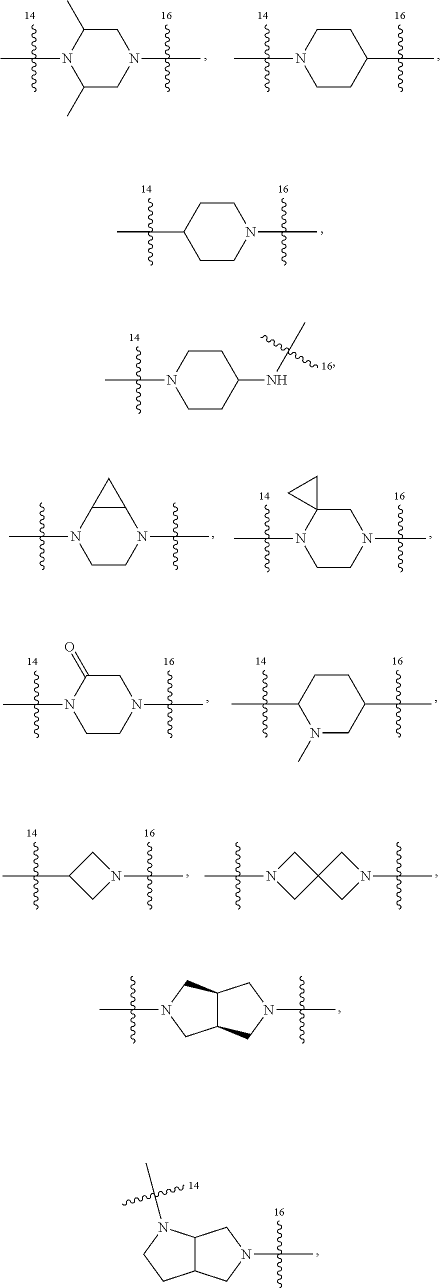 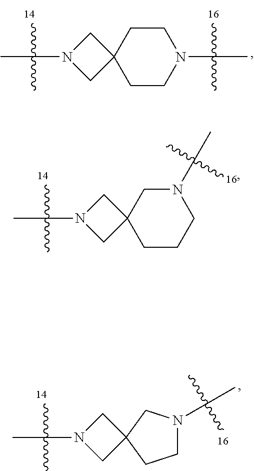 [SMILES: *N1CCC2(CC1)CN(*)C2 ; *N1CCCC2(C1)CN(*)C2 ; *N1CCC2(C1)CN(*)C2]
or is absent;
wherein [symbol] indicates attachment to R¹⁴ and [symbol] indicates attachment to R¹⁶.

20. The compound of claim 19, wherein R¹⁵ is piperazine, 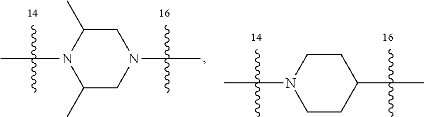
or is absent.

21. The compound of any preceding claim, wherein R¹⁶ is —C₁₋₆alkyl, —CH₂—C(O)—NH—, —CH₂—C(O)—, —C(O)— or is absent.

22. The compound of any preceding claim, wherein R¹⁷ is absent.

23. The compound of any preceding claim, wherein R¹⁸ is —C₁₋₆ alkyl, cyclobutyl, CH₂—NH—C(O)—, piperazine or is absent.

24. The compound of any preceding claim, wherein [linker] is selected from 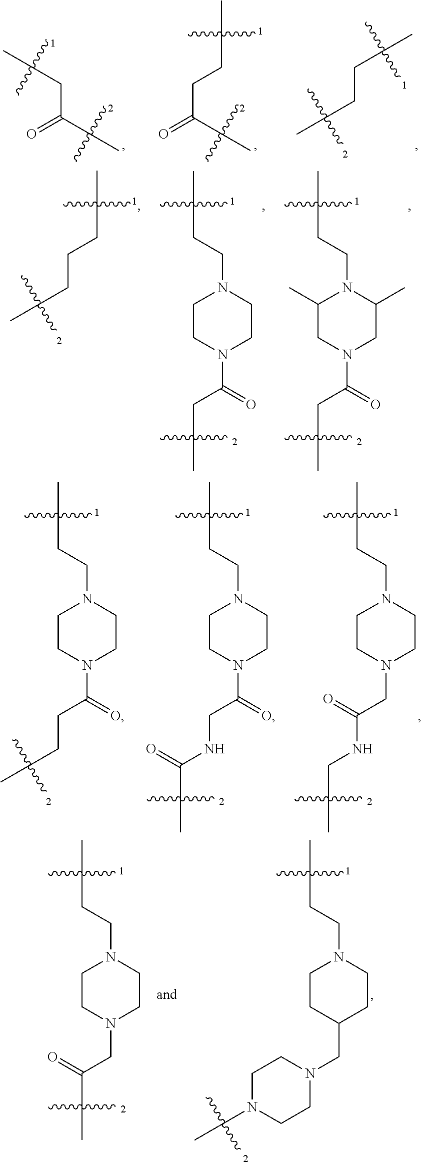
wherein [symbol]
indicates attachment to [MCL-1 ligand moiety] and [symbol]
indicates attachment to [ligase ligand moiety].

25. The compound of claim 24, wherein [linker] is selected from

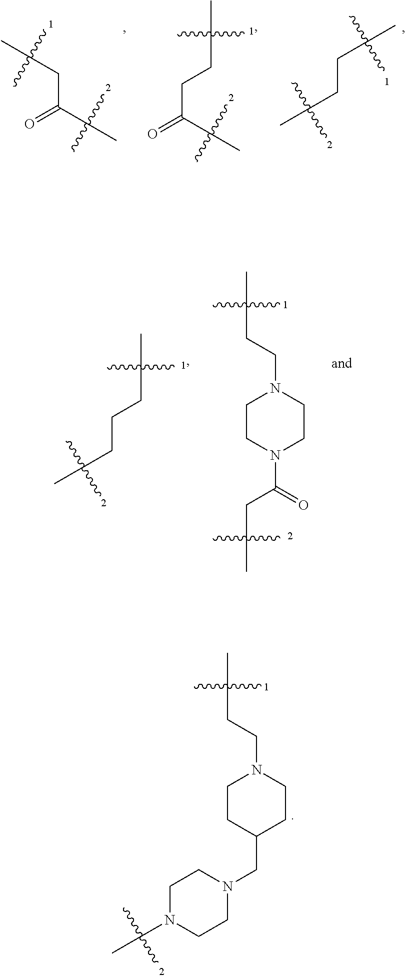

26. The compound of any preceding claim, wherein [MCL-1 ligand moiety] is of Formula (A1):

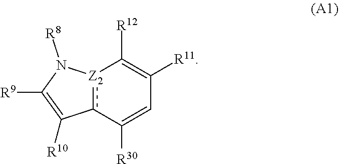

27. The compound of any one of claims 1-25, wherein [MCL-1 ligand moiety] is of Formula (A2):

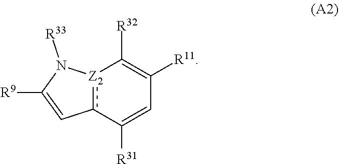

28. The compound of any one of claims 1-25, wherein [MCL-1 ligand moiety] is of Formula (A3):

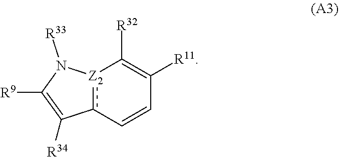

29. The compound of any one of claims 1-25, wherein [MCL-1 ligand moiety] is of Formula (A4):

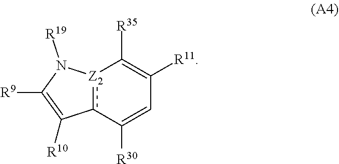

30. The compound of any preceding claim wherein in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

31. The compound of any preceding claim, wherein in Formula (A1) R¹² is 19

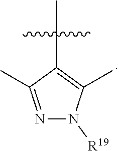

32. The compound of any preceding claim, wherein in Formula (A4) R³⁵ is

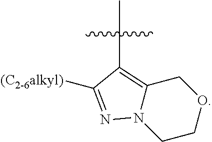

33. The compound of any preceding claim, wherein in Formula (A2) R³¹ is —C₂₋₅alkyl-O—R¹³ or —O—C₂₋₅-alkyl-R¹³, wherein R¹³ is phenyl or naphthyl, wherein the phenyl or naphthyl is optionally substituted with at least one substituent selected from halogen and C₁-C₆ alkyl.

34. The compound of any preceding claim, wherein in Formula (A3) R³⁴ is C₂₋₅alkyl-O—R¹³ or —O—C₂₋₅alkyl-R³, wherein R¹³ is phenyl or naphthyl, wherein the phenyl or naphthyl is optionally substituted with at least one substituent selected from halogen and C₁-C₆ alkyl.

35. The compound of any preceding claim, wherein Z₂ is C and [symbol] is a double bond.

36. The compound of any preceding claim, wherein each R⁹ is independently —C(O)OH or —P(O)(OH)₂.

37. The compound of any preceding claim, wherein R¹¹ is halogen.

38. The compound of any preceding claim, wherein each R²⁰ is Me or —(CH₂CH₂O)₂Me.

39. The compound of any preceding claim, wherein the C₁-C₆ alkyl substituted with morpholine or a piperazine is

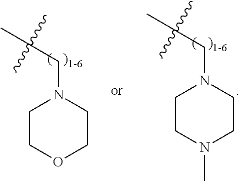

40. The compound of any preceding claim, wherein [MCL-1 ligand moiety] is selected from:

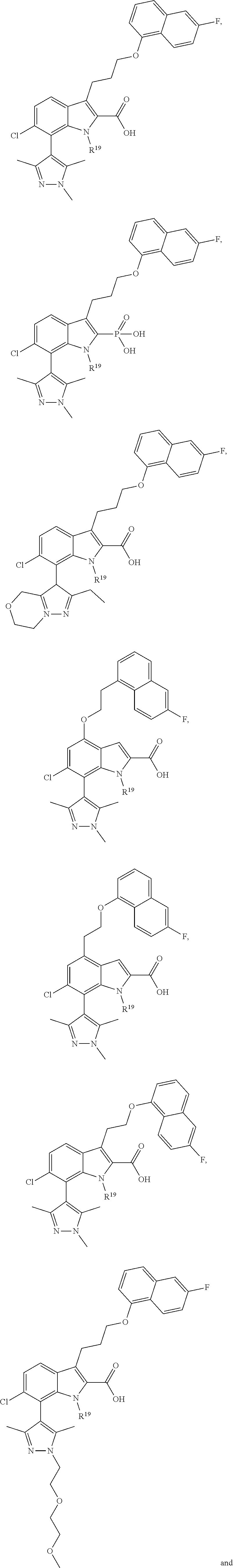

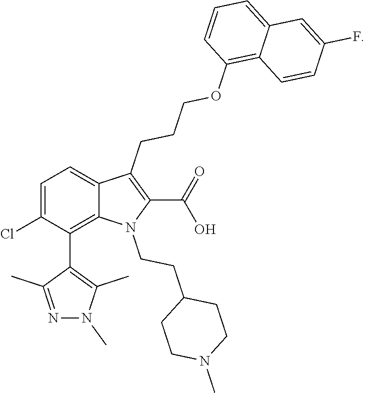 [SMILES: Cc1nn(C)c(C)c1-c1c(Cl)ccc2c(CCCOc3cccc4cc(F)ccc34)c(C(=O)O)n(CCC3CCN(C)CC3)c12]

41. The compound of claim 1, which is selected from:

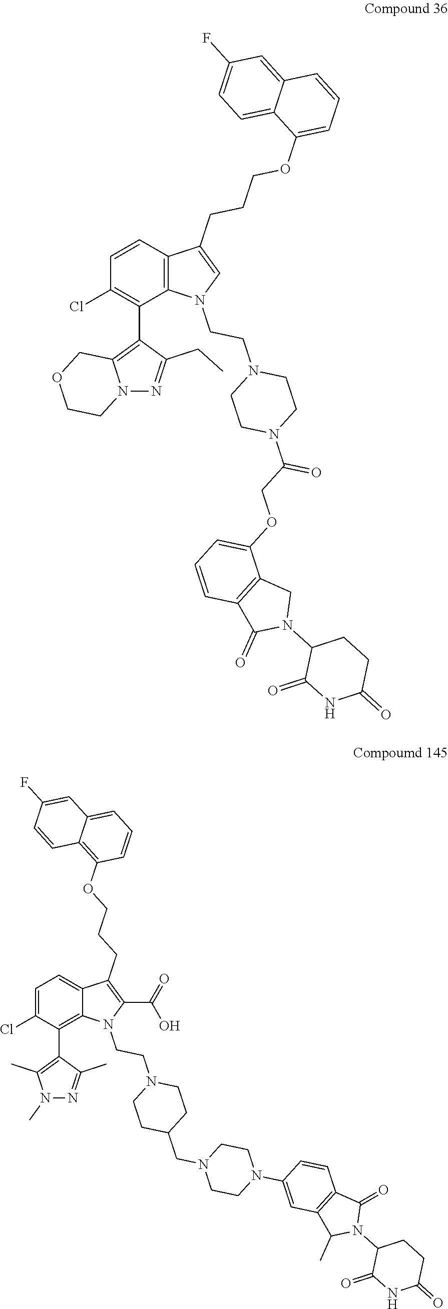 [SMILES: CCc1nn2c(c1-c1c(Cl)ccc3c(CCCOc4cccc5cc(F)ccc45)cn(CCN4CCN(C(=O)COc5cccc6c5CN(C5CCC(=O)NC5=O)C6=O)CC4)c13)COCC2 ; Cc1nn(C)c(C)c1-c1c(Cl)ccc2c(CCCOc3cccc4cc(F)ccc34)c(C(=O)O)n(CCN3CCC(CN4CCN(c5ccc6c(c5)C(C)N(C5CCC(=O)NC5=O)C6=O)CC4)CC3)c12]

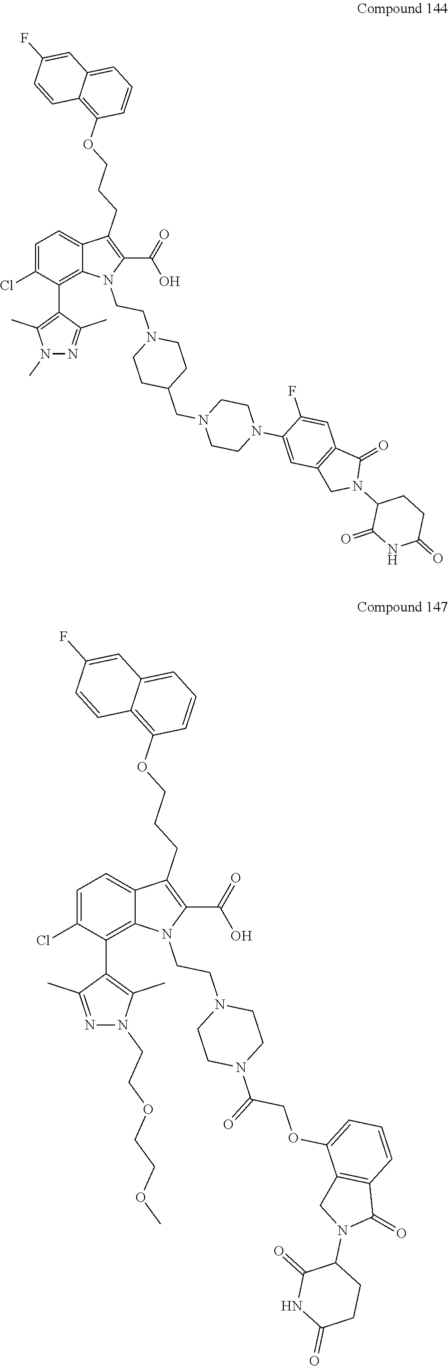 [SMILES: Cc1nn(C)c(C)c1-c1c(Cl)ccc2c(CCCOc3cccc4cc(F)ccc34)c(C(=O)O)n(CCN3CCC(CN4CCN(c5cc6c(cc5F)C(=O)N(C5CCC(=O)NC5=O)C6)CC4)CC3)c12 ; COCCOCCn1nc(C)c(-c2c(Cl)ccc3c(CCCOc4cccc5cc(F)ccc45)c(C(=O)O)n(CCN4CCN(C(=O)COc5cccc6c5CN(C5CCC(=O)NC5=O)C6=O)CC4)c23)c1C]

42. The compound of claim 41, wherein the compound is:
(a) Compound 144 or 147; or
(b) Compound 147

43. A compound of formula (I) 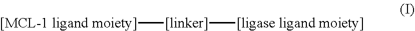
or a salt, solvate, hydrate, isomer or prodrug thereof,
wherein [ligase ligand moiety] is:
(a) Formula (IV) or (IVa) 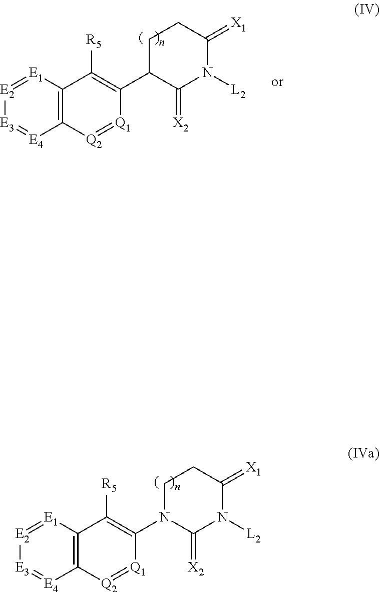
wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
and
each R^{b} is independently hydrogen, alkyl, alkenyl, aryl, heteroaryl, or benzyl;
wherein R²¹ is a bond connected to R¹⁸ of the linker, and wherein Formula (IV) contains a single R²¹;
(b) Formula (VIIa), (VIIb), (VIIc) or (VIId): 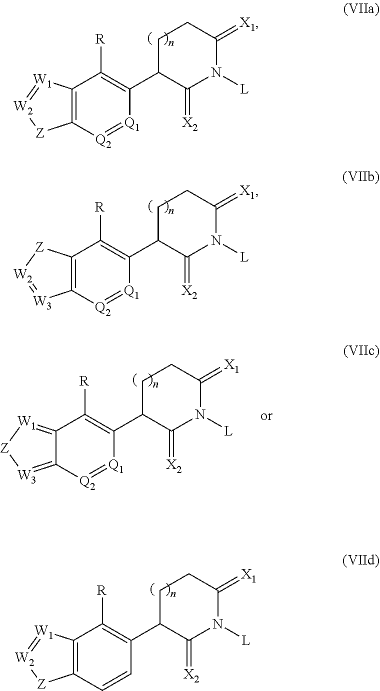
wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR*;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII): 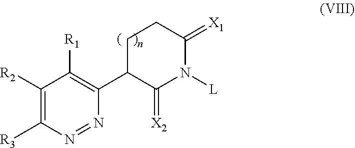
wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX): 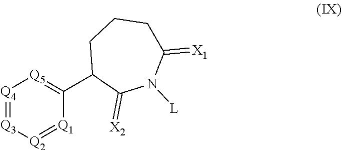
wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand moiety] is a compound of Formula (A1), (A2), (A3) or (A4): 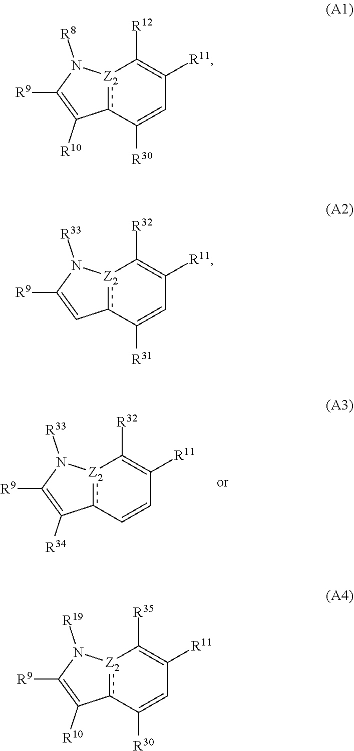
wherein [symbol]
is a single bond or a double bond;
each Z₂ is independently N or C, wherein when Z₂ is N, then [symbol] is a single bond; and when Z₂ is C, then [symbol] is a double bond;
each R⁹ is independently —C(O)OH, —C(O)OC₁-C₆alkyl, —C(O)NH₂ or —P(O)(OH)₂,
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—, 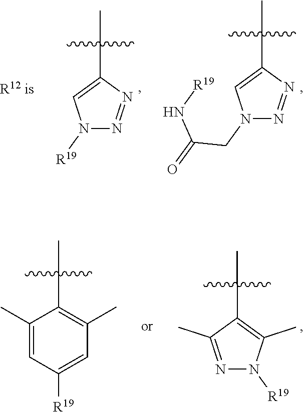
each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 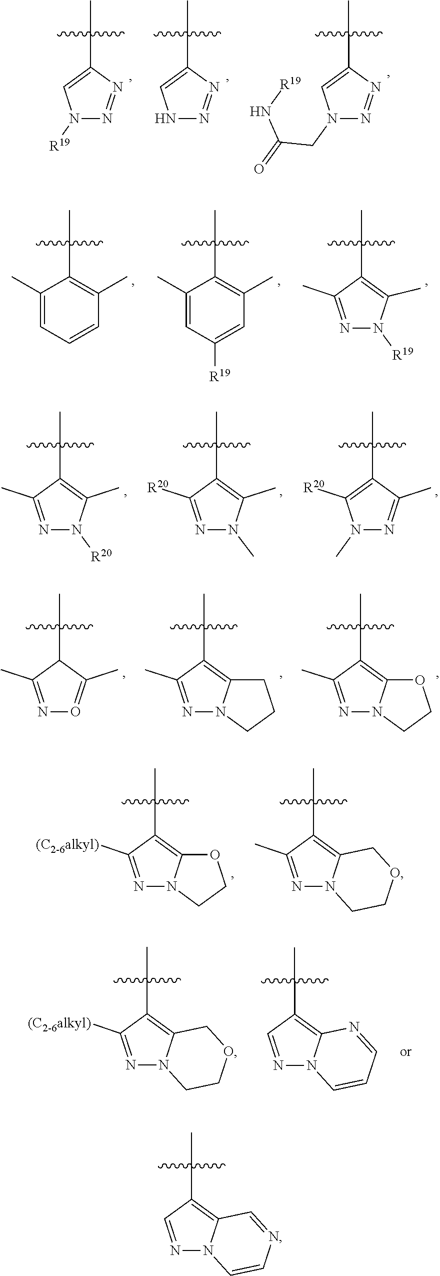
wherein R²⁰ is Me, —CH₂—OMe, —(CH₂CH₂O)ₚ(C₁-C₆ alkyl), —CH₂—O-bromobenzaldehyde, or 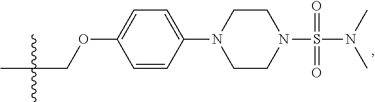
wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is or 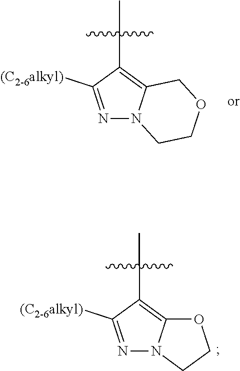
and
R¹⁹ is a bond connected to R¹⁴ of the linker, wherein each of Formula (A1), Formula (A2), Formula (A3) and Formula (A4) contains a single R¹⁹;
and wherein [linker] has the following formula 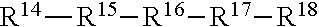
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl, —C₁₋₆ alkyl-C(O)—, cycloalkyl, CH₂—NH—C(O)—, heterocycloalkyl, or is absent
wherein at least one of R¹⁴-R¹⁸ is present.

44. The compound of claim 43, wherein
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent; and
R¹⁸ is —C₁₋₆ alkyl, cycloalkyl, CH₂—NH—C(O)—, heterocycloalkyl, or is absent.

45. The compound of claim 43 or 44, wherein [linker] is 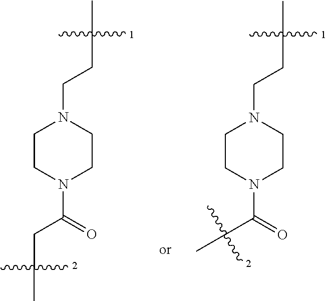 [SMILES: *CCN1CCN(C(=O)C*)CC1 ; *CCN1CCN(C(*)=O)CC1]
wherein [symbol]
indicates attachment to [MCL-1 ligand moiety] and [symbol]
indicates attachment to [ligase ligand moiety].

46. The compound of any one of claims 43-45, wherein [MCL-1 ligand moiety] is

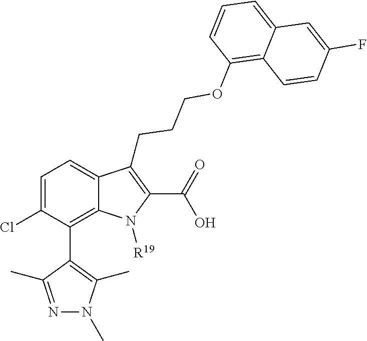

47. The compound of any one of claims 44-46, wherein [ligase ligand moiety] is:

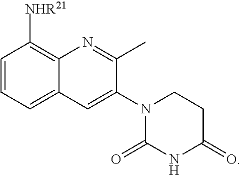

48. The compound of claim 47, wherein the compound is

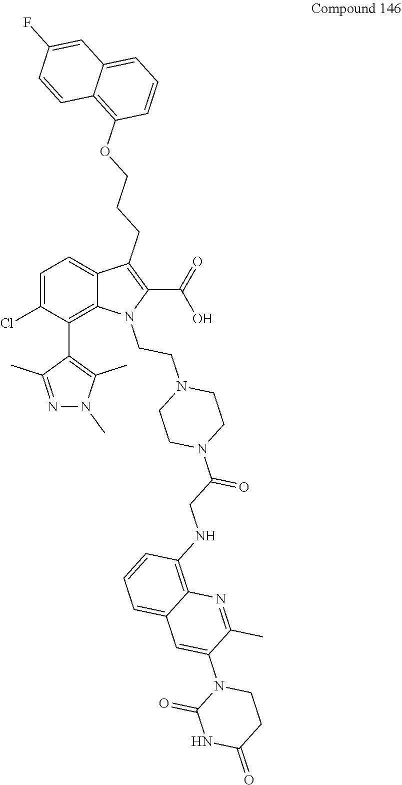

49. The compound of any one of claims 43-46, wherein [ligase ligand moiety] is:

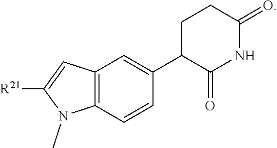

50. The compound of claim 49, wherein the compound is

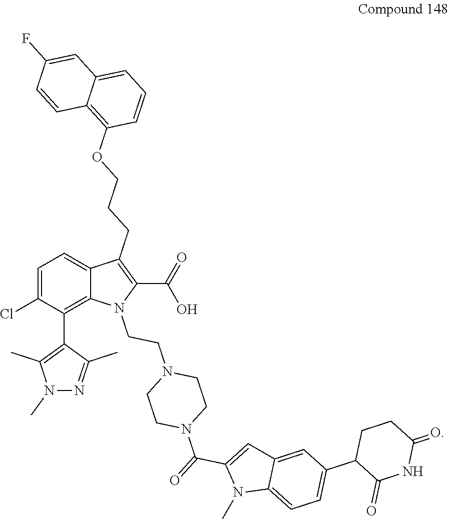 [SMILES: Cc1nn(C)c(C)c1-c1c(Cl)ccc2c(CCCOc3cccc4cc(F)ccc34)c(C(=O)O)n(CCN3CCN(C(=O)c4cc5cc(C6CCC(=O)NC6=O)ccc5n4C)CC3)c12]

51. A compound of formula (I) 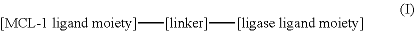
or a salt, solvate, hydrate, isomer or prodrug thereof,
wherein [ligase ligand moiety] is:
(a) Formula (Va) or (Vb): 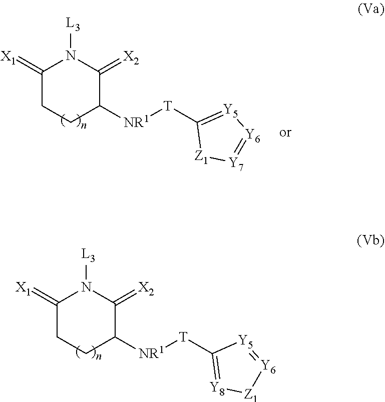
or a pharmaceutically acceptable salt or tautomer thereof,
wherein
each of X₁ and X₂ is independently O or S;
Z₁ is O, S or NR⁶;
T is is C═O or SO₂;
R¹ is hydrogen, alkyl, cycloalkyl, alkenyl, cycloalkenyl, aryl, heteroaryl, or benzyl;
each of Y₅, Y₆, Y₇, and Y₈ is independently N or CR⁷,
wherein at least one of Y₅, Y₆ and Y₇ in Formula (Va) is CR⁷, and at least one of Y₅, Y₅ and Y₈ in Formula (Vb) is CR⁷;
n is 0, 1 or 2;
L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
each R″″ is independently hydrogen, alkyl, cycloalkyl, alkenyl, cycloalkenyl, aryl, heteroaryl, or benzyl;
R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
wherein R²¹ is a bond connected to R¹⁸ of the linker, and wherein formula (Va) and formula (Vb) each contain a single R²¹;
wherein when Z₁ is 0, then Y₆ is CR⁷ and
wherein when the compound is of Formula (Va), then
(i) when each of Y₅, Y₆ and Y₇ is CR⁷, then at least one of R⁷ is not H;
(ii) when Z₁ is NR⁶, then Y₆ and Y₇ are CR⁷;
(iii) when Z₁ is S, then Y₅ is not C—OMe and Y₆ is not C—OMe;
(iv) when Z₁ is S and Y₅ is C—NHCOMe, then Y₇ is not C—CH₂NR″″C(O)OR″″;
(v) when Z₁ is S and Y₅ is N, then Y₆ is not C—H, C-aryl or C—C(O)OR″″; and
(vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
(vii) when each of Y₅, Y₆ and Y₈ is CR⁷, then at least one of R⁷ is not H;
(viii) when Z₁ is S, then Y₅ is not C—COOH or C—NHC(O)Me, and Y₈ is not C—Br;
(ix) when Z₁ is S and Y₆ is C—Br, then Y₈ is C—OR″″
(x) when Z₁ is S, Y₅ is N and Y₆ is C—H or C—NH₂, then Y₈ is not C—H
(xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
or
(b) Formula (IIa) or (IIb): 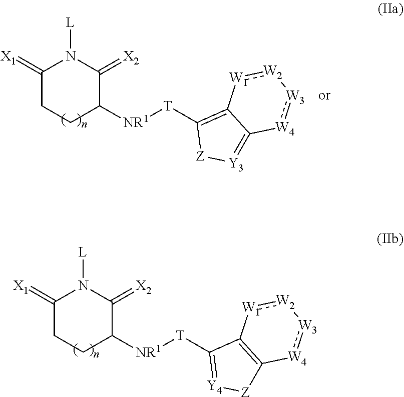
wherein
each of X₁ and X₂ is independently O or S;
Z is O, S or NR²;
T is C═O or SO₂;
Y₃ is N or CR;
Y₄ is N or CR; [symbol]
indicates a single or double bond, wherein
when each [symbol] is a double bond, each of W₁, W₂, W₃ and W₄ is independently N or CR^{a}, wherein at least one of W₁, W₂, W₃ and W₄ is N, and
when each [symbol] is a single bond, W₁, W₂, W₃ and W₄ are each CR^{a2} and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 igand moiety] is a compound of Formula (A1) (A2) (A3) or (A4): 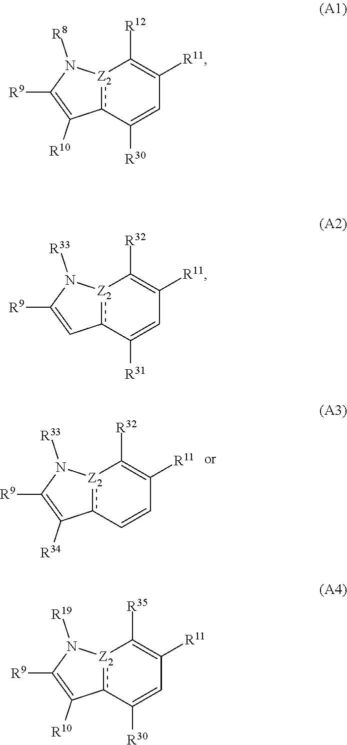
wherein [symbol]
is a single bond or a double bond;
each Z₂ is independently N or C, wherein when Z₂ is N, then [symbol] is a single bond; and when Z₂ is C, then [symbol] is a double bond;
each R⁹ is independently —C(O)OH, —C(O)OC₁-C₆alkyl, —C(O)NH₂ or —P(O)(OH)₂,
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 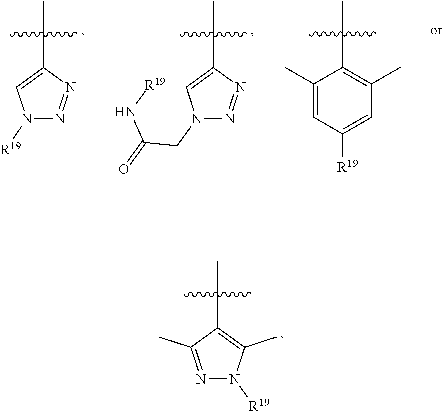
each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 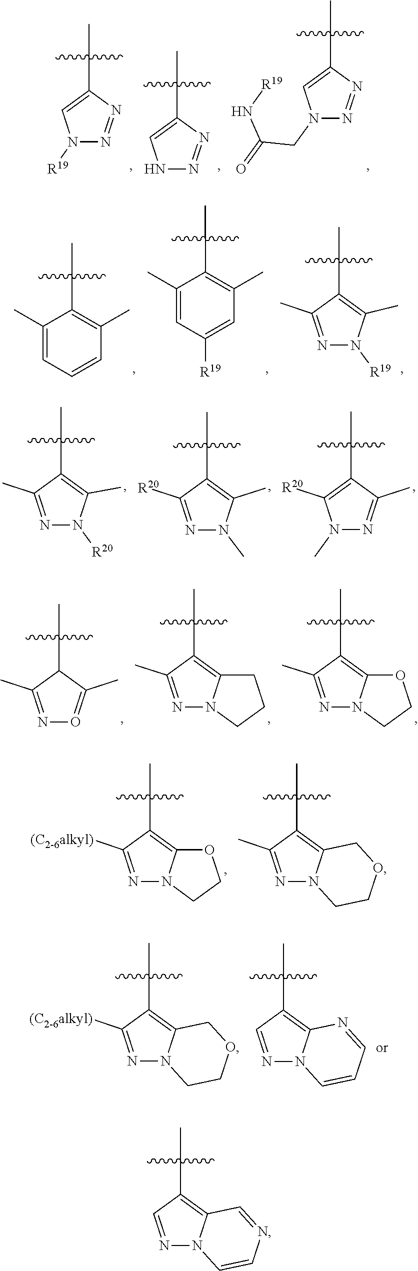
wherein R²⁰ is Me, —CH₂—OMe, —(CH₂CH₂O)ₚ(C₁-C₆ alkyl), —CH₂—O-bromobenzaldehyde, or 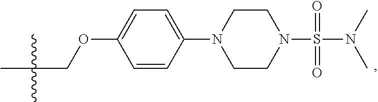
wherein p is 1-5;
R³⁴ is C₂₋₅alkyl-O—R¹³ or —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is 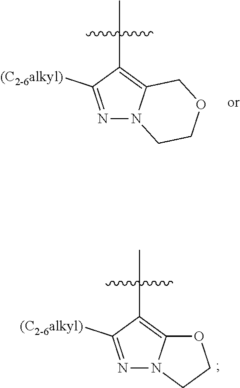
and
R¹⁹ is a bond connected to R¹⁴ of the linker, wherein each of Formula (A1), Formula (A2), Formula (A3) and Formula (A4) contains a single R¹⁹;
and wherein [linker] has the following formula 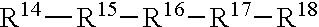
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl, —C₁₋₆ alkyl-C(O)—, cycloalkyl, CH₂—NH—C(O)—, heterocycloalkyl, or is absent wherein at least one of R¹⁴-R¹⁸ is present.

52. A compound of formula (I) 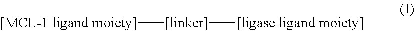
or a salt, solvate, hydrate, isomer or prodrug thereof,
wherein [ligase ligand moiety] is:
(a) Formula (VIa) or (VIb): 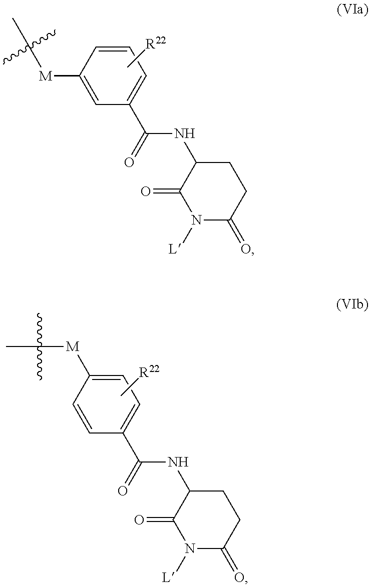
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl; and
L′ is hydrogen, alkyl, benzyl, acetyl or pivaloyl
(b) Formula (II): 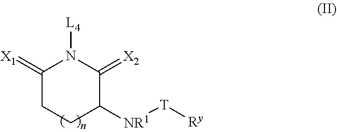
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from 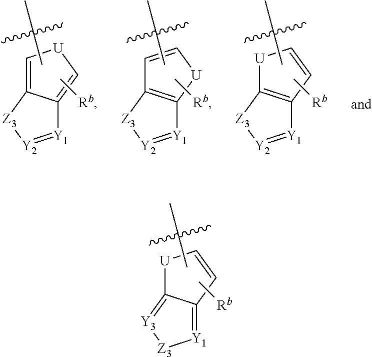
wherein [symbol] indicates attachment to T,
Z₃ is O, S or NR³;
U is O, S, NR^{b} or CRⁱ ₂;
each of Y₁, Y₂ and Y₃ is independently N or CR^{d};
each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
each R^{b} is independently alkyl, cycloalkyl, alkenyl, cycloalkenyl, aryl, heteroaryl, or benzyl;
R²¹ is a bond connected to R¹⁸ of the linker, wherein Formula (II) contains a single R²¹;
wherein,
(i) when R^{y} is 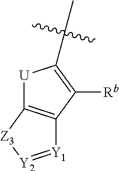
then Y₂ is CR^{d}; and
(ii) when R^{y} is 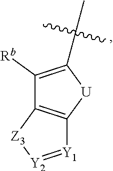
then Rⁱ in CRⁱ₂ is not hydrogen
or
(c) Formula (III): 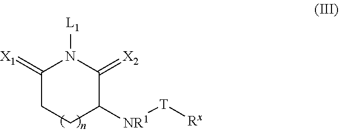
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from 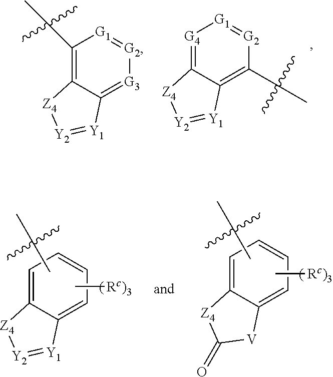
wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{C}, then C=X₁ may be replaced by CH;
and wherein:
(i) when R^{x} is 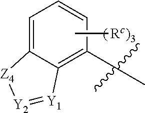
and Z₄ is NH, then L₁ is hydrogen, —CH₂C(O)OR^{b}, or —OR^{b};
(ii) when R^{x} is 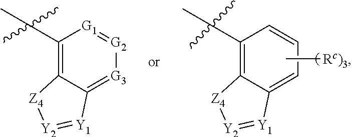
Z₄ is NR⁴, Y₁ is CR^{f}, and Y₂ is N, then R⁴ is not alkyl and at least one of R² and R is not H;
(iii) when R^{x} is 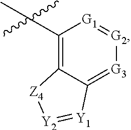
Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not 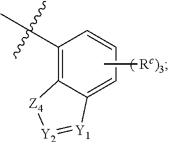
(v) when R^{x} is 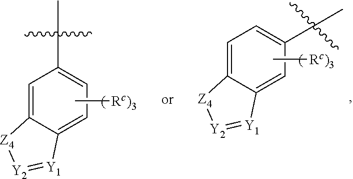
Z₄ is NR⁴, and Y₁ or Y₂ is N, then R⁴ is not alkyl;
(vi) when R^{x} is 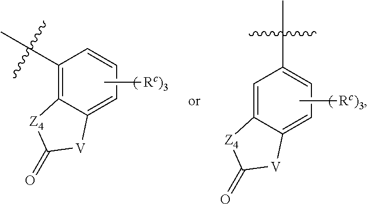
then n=1 or 2; and
(vii) when R^{x} is 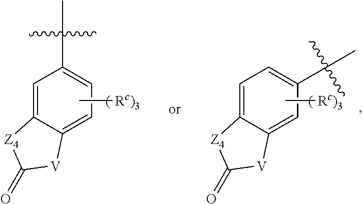
then Z₄=O or S
wherein [MCL-1 ligand moiety] is a compound of Formula (A1), (A2), (A3) or (A4): 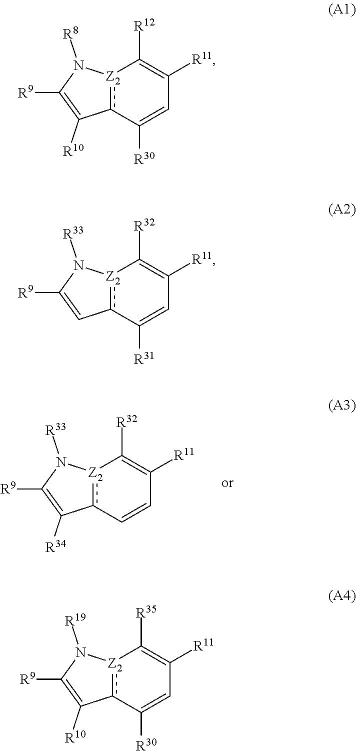
wherein [symbol]
is a single bond or a double bond;
each Z₂ is independently N or C, wherein when Z₂ is N, then [symbol] is a single bond; and when Z₂ is C, then [symbol] is a double bond;
each R⁹ is independently —C(O)OH, —C(O)OC₁-C₆alkyl, —C(O)NH₂ or —P(O)(OH)₂,
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 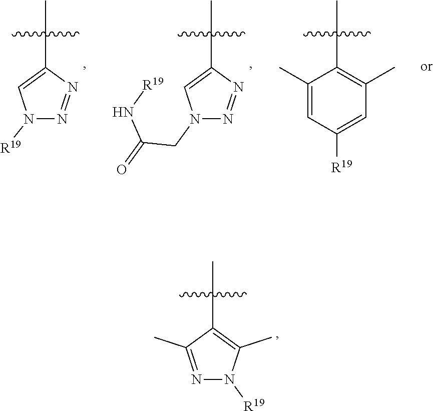
each R³³ is independently H, R¹⁹, or C₁-C₆ alkyl optionally substituted with morpholine or a piperazine;
R³¹ is —C₂₋₅alkyl-O—R¹³, —O—C₂₋₅alkyl-R¹³ or —C₂₋₅alkyl-NMe-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 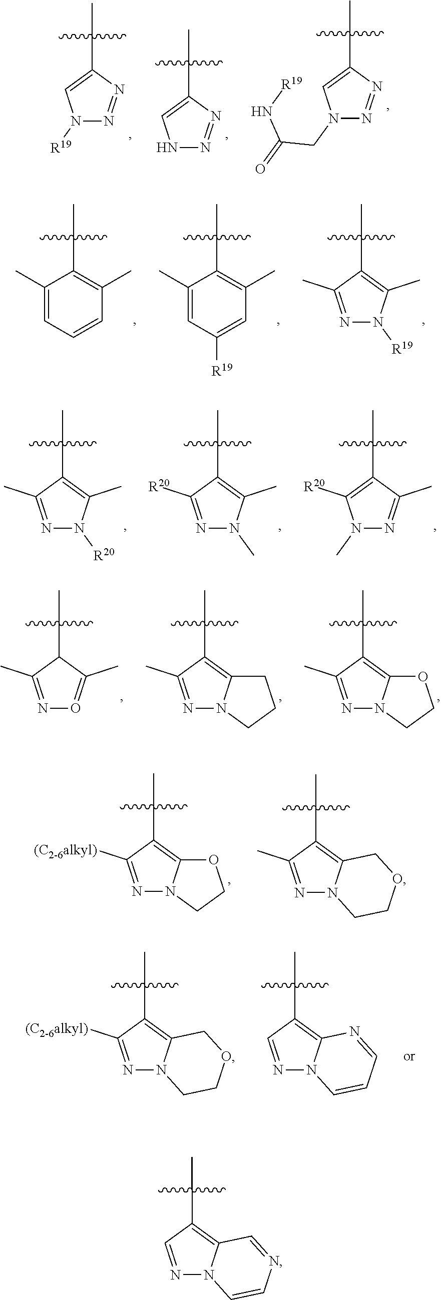
wherein R²⁰ is Me, —CH₂—OMe, —(CH₂CH₂O)ₚ(C₁-C₆ alkyl), —CH₂—O-bromobenzaldehyde, or 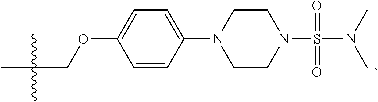
wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is 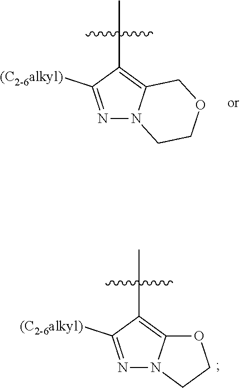
and
R¹⁹ is a bond connected to R¹⁴ of the linker, wherein each of Formula (A1), Formula (A2), Formula (A3) and Formula (A4) contains a single R¹⁹;
and wherein [linker] has the following formula 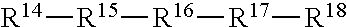
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl, —C₁₋₆ alkyl-C(O)—, cycloalkyl, CH₂—NH—C(O)—, heterocycloalkyl, or is absent
wherein at least one of R¹⁴-R¹⁸ is present.

53. The compound of claim 52, wherein [ligase ligand moiety] is

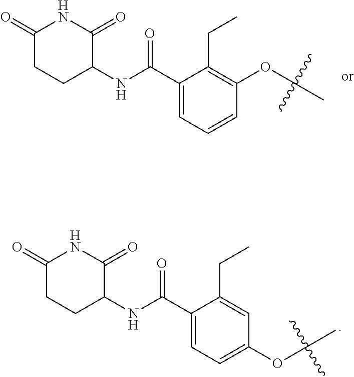 [SMILES: *Oc1cccc(C(=O)NC2CCC(=O)NC2=O)c1CC ; *Oc1ccc(C(=O)NC2CCC(=O)NC2=O)c(CC)c1]

54. The compound of any one of claims 51-53, wherein
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent; and
R¹⁸ is —C₁₋₆ alkyl, cycloalkyl, CH₂—NH—C(O)—, heterocycloalkyl, or is absent.

55. The compound of claim 53 or 54, wherein [linker] is 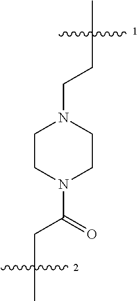 [SMILES: *CCN1CCN(C(=O)C*)CC1]
wherein [symbol]
indicates attachment to [MCL-1 ligand moiety] and [symbol]
indicates attachment to [ligase ligand moiety].

56. The compound of any one of claims 51-55, wherein [MCL-1 ligand moiety] is

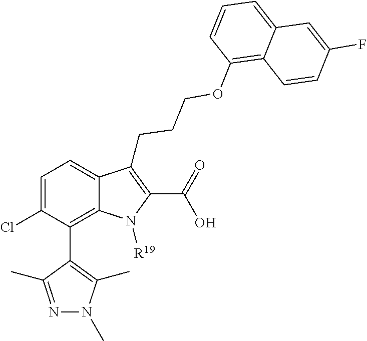

57. The compound of claim 56, wherein the compound is

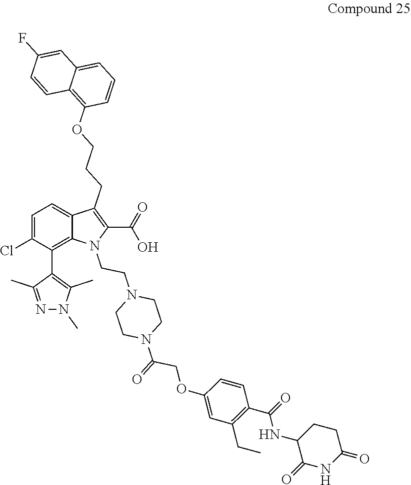 [SMILES: CCc1cc(OCC(=O)N2CCN(CCn3c(C(=O)O)c(CCCOc4cccc5cc(F)ccc45)c4ccc(Cl)c(-c5c(C)nn(C)c5C)c43)CC2)ccc1C(=O)NC1CCC(=O)NC1=O]

58. The compound of claim 52, wherein [ligase ligand moiety] is Formula (III):

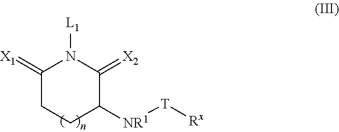

59. The compound of claim 58, wherein [ligase ligand moiety] is

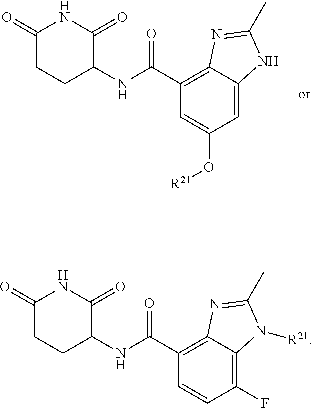

60. A compound of formula (I) 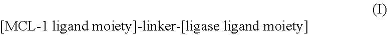
or a salt, solvate, hydrate, isomer or prodrug thereof,
wherein [ligase ligand moiety] is: 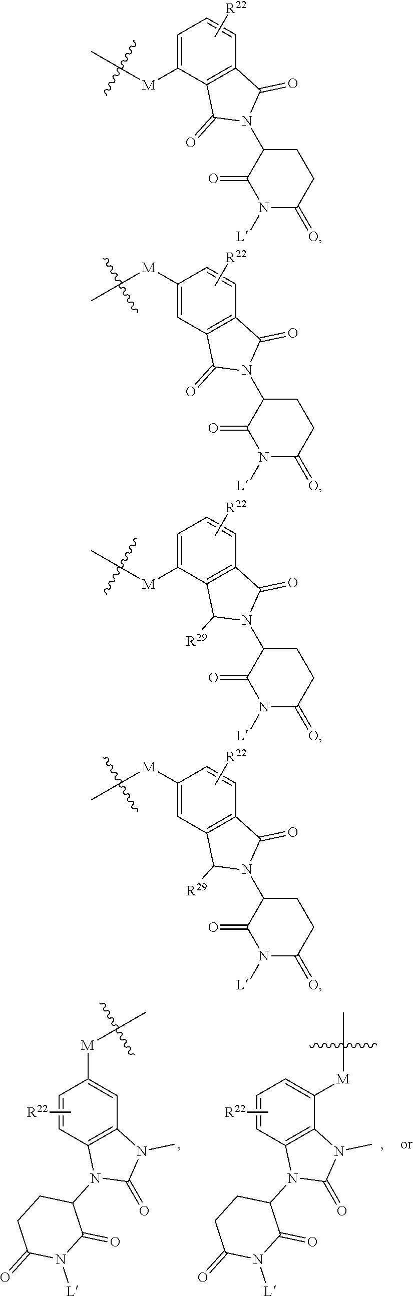 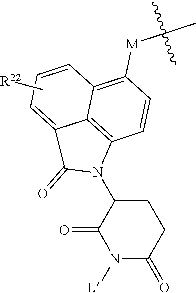
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl; and
R²⁹ is hydrogen or Me
L′ is hydrogen, alkyl, benzyl, acetyl or pivaloyl;
wherein [MCL-1 ligand moiety] is a compound of Formula (A): 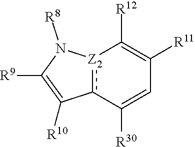
wherein [symbol]
is a single bond or a double bond;
R⁸ is H, R¹⁹, or C₁-C₆ alkyl optionally substituted with morpholine or a piperazine;
R⁹ is —C(O)OH, —C(O)OC₁-C₆alkyl, —C(O)NH₂ or —P(O)(OH)₂,
one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H, 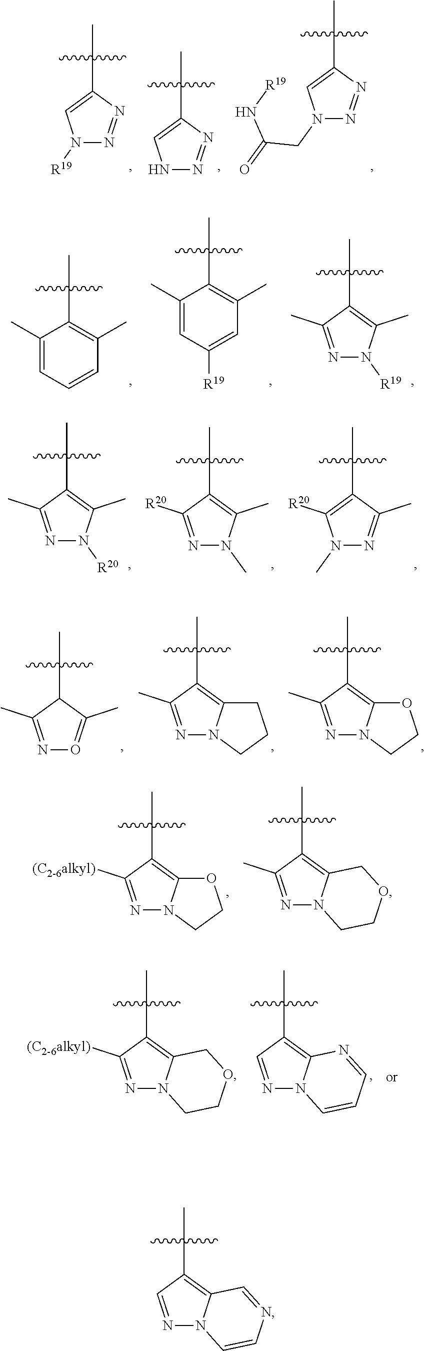
wherein R²⁰ is Me, —CH₂—OMe, —(CH₂CH₂O)ₚ(C₁-C₆ alkyl), or —CH₂—O-bromobenzaldehyde, wherein p is 1-5; or when R¹² is 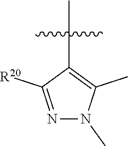
and R¹⁰ is —O-naphthyl substituted with —O— or —S—, then R²⁰ is 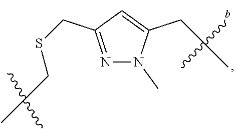 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
Z₂ is N or C, wherein when Z₂ is N, then [symbol] is a single bond; and when Z₂ is C, then [symbol] is a double bond,
and wherein Formula (A) contains a single R¹⁹;
and wherein [linker] has the following formula 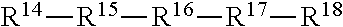
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl-C(O)—, cycloalkyl or —CH₂—NH—C(O)—.

61. The compound of claim 60, wherein [ligase ligand moiety] is

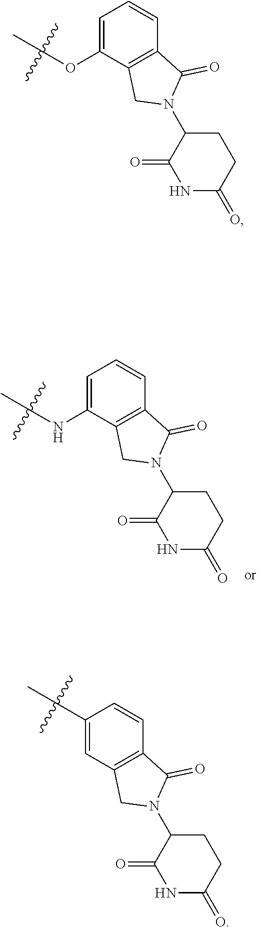 [SMILES: *Oc1cccc2c1CN(C1CCC(=O)NC1=O)C2=O ; *Nc1cccc2c1CN(C1CCC(=O)NC1=O)C2=O ; *c1ccc2c(c1)CN(C1CCC(=O)NC1=O)C2=O]

62. The compound of claim 60 or 61, wherein
R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent
R¹⁸ is cycloalkyl or —CH₂—NH—C(O)—.

63. The compound of any one of claims 60-62, wherein R¹⁴ is —C₁₋₆alkyl, —C₂₋₆alkenyl, —C₂₋₆alkynyl, —C(O)—, —SO₂— or is absent.

64. The compound of claim 63, wherein R¹⁴ is —C₁₋₆ alkyl.

65. The compound of any one of claims 60-64, wherein R¹⁵ is heterocycloalkyl.

66. The compound of claim 65, wherein R¹⁵ is piperazine, 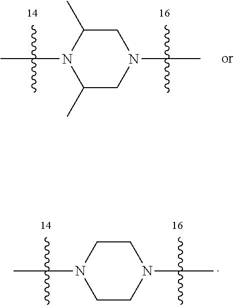
wherein [symbol] indicates attachment to R¹⁴ and [symbol] indicates attachment to R¹⁶.

67. The compound of any one of claims 60-66, wherein R¹⁶ is —C₁₋₆ alkyl, —CH₂—C(O)— or —C(O)—.

68. The compound of any one of claims 60-67, wherein R¹⁷ is absent.

69. The compound of any one of claims 60-68, wherein R¹⁸ is cyclobutyl.

70. The compound of any one of claims 59-69, wherein [MCL-1 ligand moiety] is

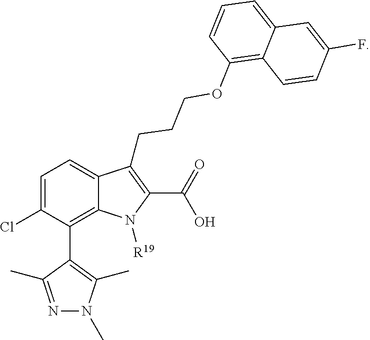

71. A compound of formula (I) 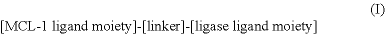
or a salt, solvate, hydrate, isomer or prodrug thereof,
wherein [ligase ligand moiety] is:
(a) Formula (IV) or (IVa) 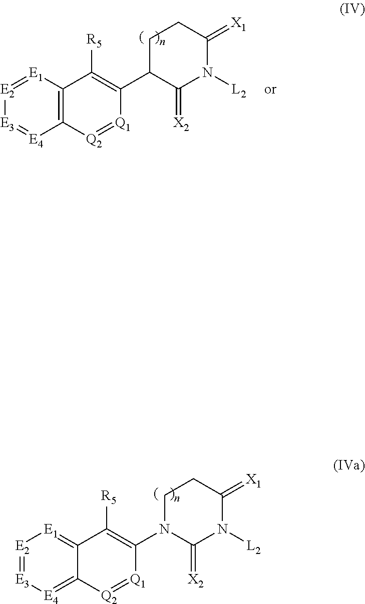
wherein:
each of X₁ and X₂ is independently O or S;
each of Q₁ and Q₂ is independently N or CR⁵, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IV) contains a single R²¹;
(b) Formula (VIIa), (VIIb), (VIIc) or (VIId): 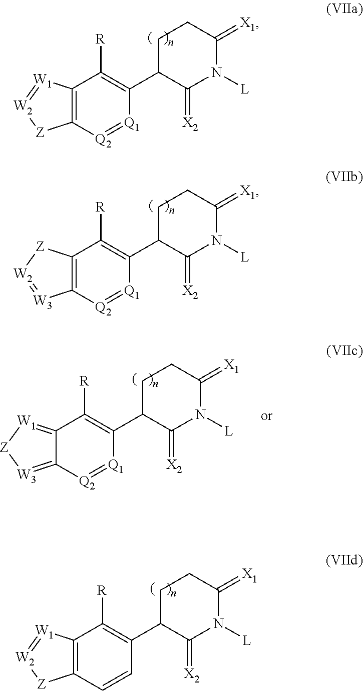
wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR*;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII): 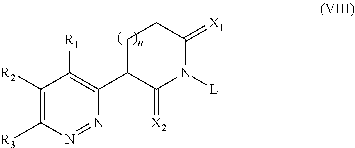
wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX): 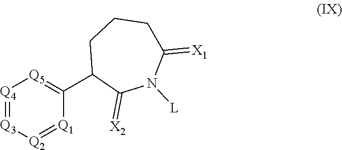
wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand moiety] is a compound of Formula (A): 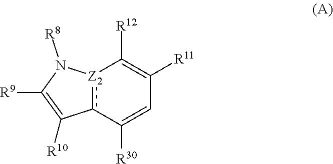
wherein [symbol]
is a single bond or a double bond;
R⁸ is H, R¹⁹, or C₁-C₆ alkyl optionally substituted with morpholine or a piperazine;
R⁹ is —C(O)OH, —C(O)OC₁-C₆alkyl, —C(O)NH₂ or —P(O)(OH)₂,
one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl, 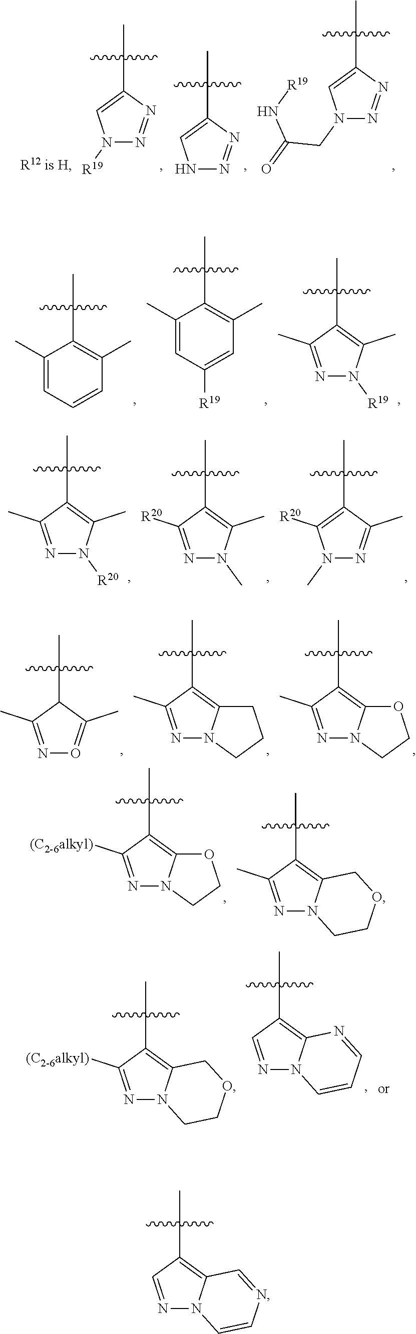
wherein R²⁰ is Me, —CH₂—OMe, —(CH₂CH₂O)ₚ(C₁-C₆ alkyl), —CH₂—O-bromobenzaldehyde, or 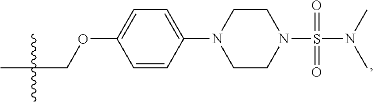
wherein p is 1-5;
or when R¹² is 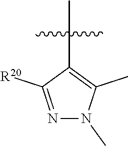
and R¹⁰ is —O-naphthyl substituted with —O— or —S—, then R²⁰ is 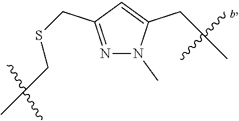 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
Z₂ is N or C, wherein when Z₂ is N, then [symbol] is a single bond; and when Z₂ is C, then [symbol] is a double bond,
and wherein Formula (A) contains a single R¹⁹;
and wherein [linker] has the following formula 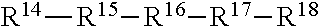
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl-C(O)—, cycloalkyl or —CH₂—NH—C(O)—.

72. The compound of claim 71, wherein [ligase ligand moiety] is Formula (IV):

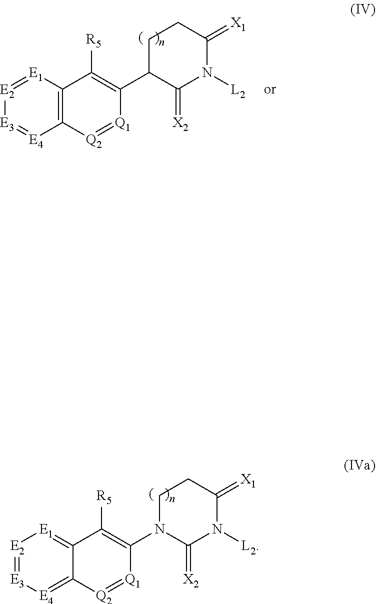

73. The compound of claim 71 or 72, wherein each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹.

74. The compound of claim 73, wherein each R^{b} is independently hydrogen, alkyl, cycloalkyl, or aryl.

75. The compound of claim 74, wherein the aryl is substituted with one or more groups selected from halogen, alkyl and O-haloalkyl, optionally wherein the halogen is Cl, the alkyl is methyl and the O-haloalkyl is O—CF₃.

76. The compound of any one of claims 71-75, wherein one of E₁, E₂, E₃ and E₄ is N, and the remaining three of E₁, E₂, E₃ and E₄ are each CR′;
optionally wherein:
(a) E₁ is N, and E₂, E₃ and E₄ are CR′; or
(b) E₂ is N, and E₁, E₃ and E₄ are CR′; or
(c) E₃ is N, and E₁, E₂ and E₄ are CR′; or
(d) E₄ is N, and E₁, E₂ and E₃ are CR′.

77. The compound of any one of claims 71-75, wherein E₁, E₂, E₃ and E₄ are each CR′, optionally wherein E₁, E₂, E₃ and E₄ are each CH.

78. The compound of claim 77, wherein three of E₁, E₂, E₃ and E₄ are CH, and one of E₁, E₂, E₃ and E₄ is C-halogen, C-alkyl, C-alkenyl, C-alkynyl, C-aryl, C-heteroaryl, C-benzyl, C-haloalkyl, C-haloalkenyl, C—NH₂, C—NHR^{b}, C—NR^{b}₂, C—NR^{b}C(O)R^{b}, C—NR^{b}C(O)OR^{b}, C—NO₂, C—CN, C—C(O)R^{b}, C—C(O)OR^{b}, C—C(O)NH₂, C—C(O)NHR^{b}, C—C(O)NR^{b}₂, C—C(O)NHCHR^{b}₂, C—CHR^{b}NHC(O)NHR^{b}, C—CHR^{b}NHC(O)C(halogen)₂R^{b}, C—OR^{b}, C—OC(O)R^{b}, C—OC(O)OR^{b}, C—OC(O)NH₂, C—OC(O)NHR^{b}, C—OC(O)NR^{b}₂, C—SR^{b}, C—S(O)₂R^{b}, C—S(O)₂OR^{b}, C—S(O)₂NH₂, C—S(O)₂NHR^{b}, C—S(O)₂NR^{b}₂, C—NHS(O)₂R^{b}, —R²¹, —O—R²¹, —NH—R²¹, —C(O)—R²¹, —C(O)—NH—R²¹, or —CH₂—NH—C(O)—R²¹.

79. The compound of claim 78, wherein:
(a) E₂, E₃ and E₄ are each CH; or
(b) E₁, E₃ and E₄ are each CH; or
(c) E₁, E₂ and E₄ are each CH.

80. The compound of any one of claims 71-77, wherein two of E₁, E₂, E₃ and E₄ are N, and the remaining two of E₁, E₂, E₃ and E₄ are each CR′.

81. The compound of any one of claims 71-77, wherein three of E₁, E₂, E₃ and E₄ are N, and the remaining one of E₁, E₂, E₃ and E₄ is CR′.

82. The compound of any one of claims 71-81, wherein:
(a) Q₁ is N and Q₂ is CR; or
(b) Q₁ is N and Q₂ is N; or
(c) Q₁ is CR and Q₂ is N, optionally wherein Q₁ is C—H or C-alkyl, further optionally wherein Q₁ is C—H or C-Me.

83. The compound of any one of claims 71-82, wherein:
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent;
R¹⁷ is —CH₂(C₂H₄—O)_{y}, (C₂H₄—O)ₓ, (C₃H₆—O)ₓ, or is absent; and
R¹⁸ is cycloalkyl or —CH₂—NH—C(O)—.

84. The compound of any one of claims 71-83, wherein R¹⁴ is —C₁₋₆ alkyl, —C₂₋₆alkenyl, —C₂₋₆alkynyl, —C(O)—, —SO₂— or is absent

85. The compound of claim 84, wherein R¹⁴ is —C₁₋₆ alkyl.

86. The compound of any one of claims 71-85, wherein R¹⁵ is cycloalkyl, heterocycloalkyl, aryl, heteroaryl, —C₁₋₆ alkyl-NH—, -cycloalkyl-NH— or is absent.

87. The compound of claim 86, wherein R¹⁵ is heterocycloalkyl or is absent.

88. The compound of claim 87, wherein R¹⁵ is piperazine.

89. The compound of any one of claims 71-88, wherein R¹⁶ is —C(O)—.

90. The compound of any one of claims 71-89, wherein R¹⁷ is absent.

91. The compound of any one of claims 71-90, wherein R¹⁸ is —CH₂—NH—C(O)—.

92. The compound of any one of claims 71-91, wherein [linker] is 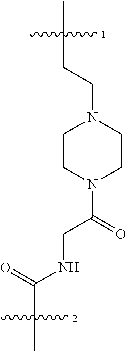 [SMILES: *CCN1CCN(C(=O)CNC(*)=O)CC1]
wherein [symbol]
indicates attachment to [MCL-1 ligand moiety] and [symbol]
indicates attachment to [ligase ligand moiety].

93. The compound of any one of claims 71-92, wherein [MCL-1 ligand moiety] is:

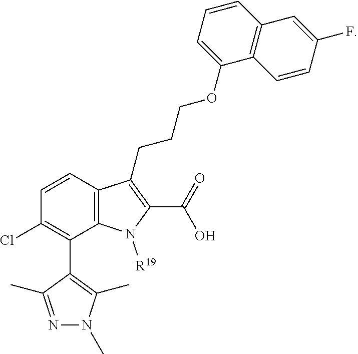

94. The compound of claim 71, wherein the compound is selected from:

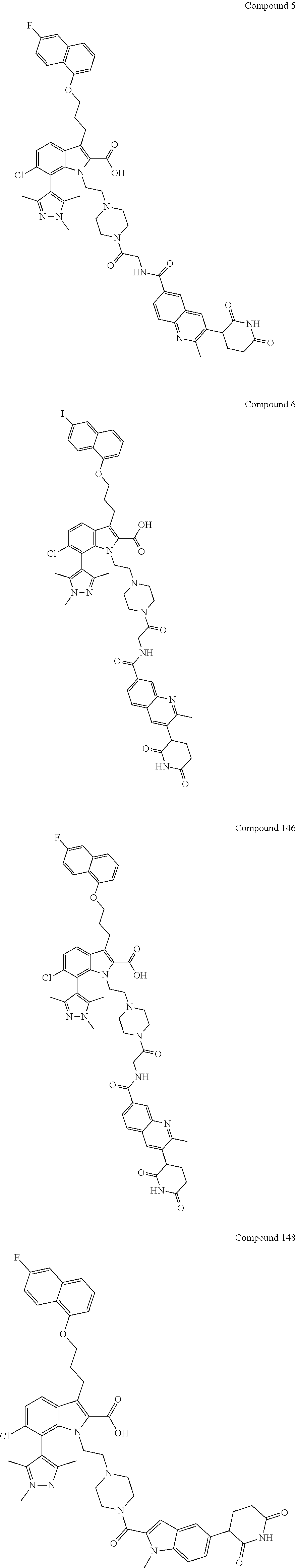

95. The compound of claim 94, wherein the compound is selected from:

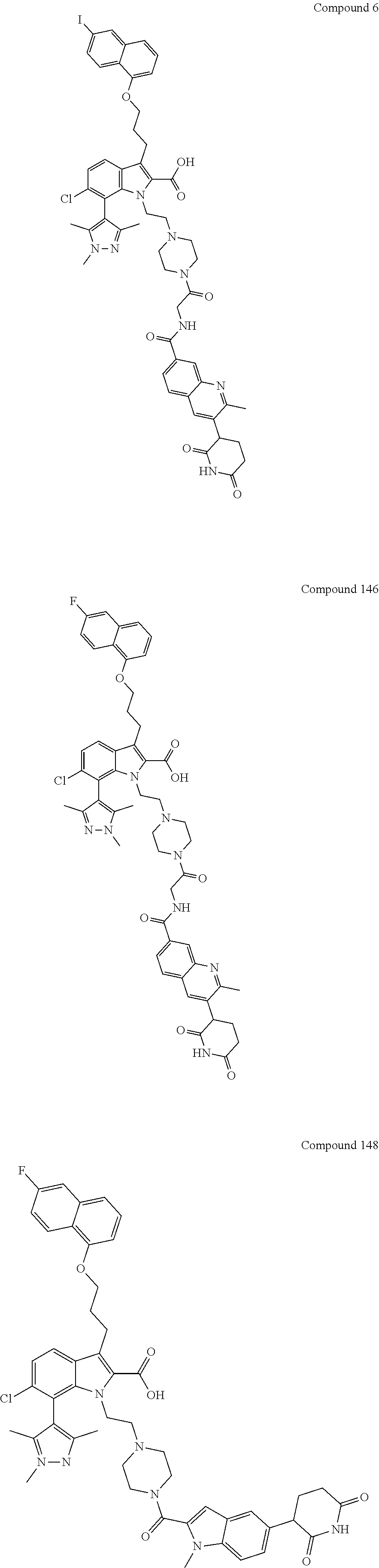

96. The compound of claim 71, wherein the compound is

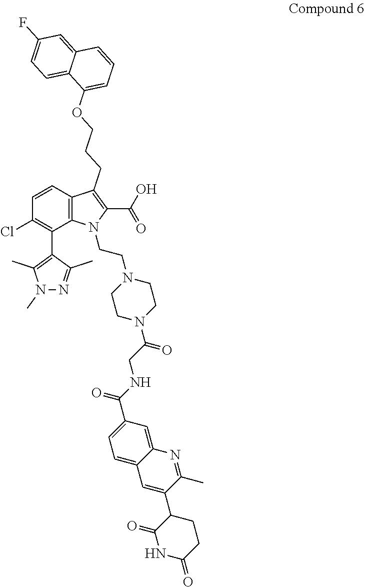

97. A compound of formula (I) 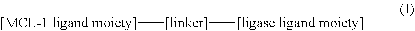
or a salt, solvate, hydrate, isomer or prodrug thereof,
wherein [ligase ligand moiety] is:
(a) Formula (Va) or (Vb): 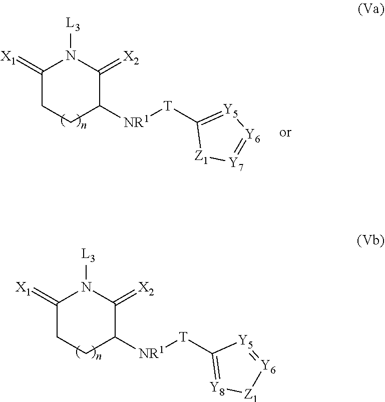
or a pharmaceutically acceptable salt or tautomer thereof,
wherein
each of X₁ and X₂ is independently O or S;
Z₁ is O, S or NR⁶;
T is is C═O or SO₂;
R¹ is hydrogen, alkyl, cycloalkyl, alkenyl, cycloalkenyl, aryl, heteroaryl, or benzyl;
each of Y₅, Y₆, Y₇, and Y₈ is independently N or CR⁷,
wherein at least one of Y₅, Y₆ and Y₇ in Formula (Va) is CR⁷, and at least one of Y₅, Y₅ and Y₈ in Formula (Vb) is CR⁷;
n is 0, 1 or 2;
L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
each R″″ is independently hydrogen, alkyl, cycloalkyl, alkenyl, cycloalkenyl, aryl, heteroaryl, or benzyl;
R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
wherein R²¹ is a bond connected to R¹⁸ of the linker, and wherein formula (Va) and formula (Vb) each contain a single R²¹;
wherein when Z₁ is 0, then Y₆ is CR⁷ and
wherein when the compound is of Formula (Va), then
(i) when each of Y₅, Y₆ and Y₇ is CR⁷, then at least one of R⁷ is not H;
(ii) when Z₁ is NR⁶, then Y₆ and Y₇ are CR⁷;
(iii) when Z₁ is S, then Y₅ is not C—Ome and Y₆ is not C—Ome;
(iv) when Z₁ is S and Y₅ is C—NHCOMe, then Y₇ is not C—CH₂NR″″C(O)OR″″;
(v) when Z₁ is S and Y₅ is N, then Y₆ is not C—H, C-aryl or C—C(O)OR″″; and
(vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
(vii) when each of Y₅, Y₆ and Y₈ is CR⁷, then at least one of R⁷ is not H;
(viii) when Z₁ is S, then Y₅ is not C—COOH or C—NHC(O)Me, and Y₈ is not C—Br;
(ix) when Z₁ is S and Y₆ is C—Br, then Y₈ is C—OR″″
(x) when Z₁ is S, Y₅ is N and Y₆ is C—H or C—NH₂, then Y₈ is not C—H
(xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
Or
(b) Formula (IIa) or (IIb): 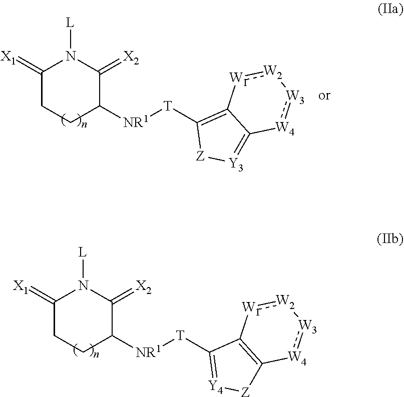
wherein
each of X₁ and X₂ is independently O or S;
Z is O, S or NR²;
T is C═O or SO₂;
Y₃ is N or CR;
Y₄ is N or CR; [symbol]
indicates a single or double bond, wherein
when each [symbol] is a double bond, each of W₁, W₂, W₃ and W₄ is independently N or CR^{a}, wherein at least one of W₁, W₂, W₃ and W₄ is N, and
when each [symbol] is a single bond, W₁, W₂, W₃ and W₄ are each CR^{a2} and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 ligand moiety] is a compound of Formula (A): 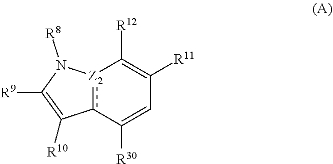
wherein [symbol]
is a single bond or a double bond;
R⁸ is H, R¹⁹, or C₁-C₆ alkyl optionally substituted with morpholine or a piperazine;
R⁹ is —C(O)OH, —C(O)OC₁-C₆alkyl, —C(O)NH₂ or —P(O)(OH)₂,
one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl);
or wherein the tetraline is optionally substituted with a bridging —CH₂— group; or wherein the naphthyl is optionally substituted with —O— or —S—,
R¹¹ is H, halogen or C₁-C₆ alkyl, 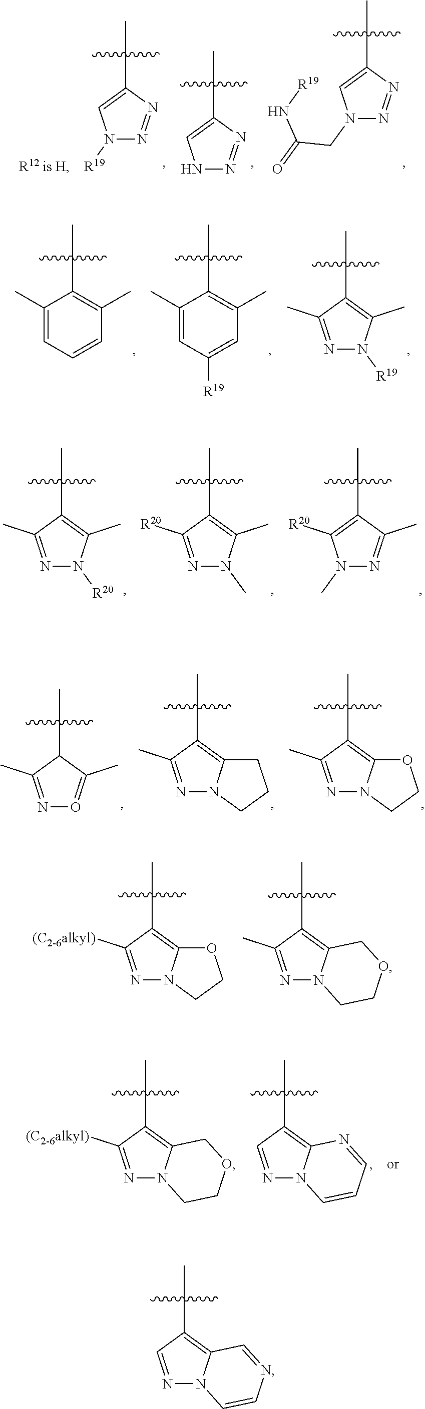
wherein R²⁰ s Me, —CH₂—OMe, —(CH₂CH₂O)ₚ(C₁-C₆ alkyl), —CH₂—O-bromobenzaldehyde, or 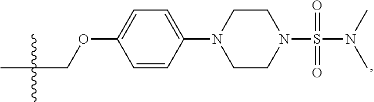
wherein p is 1-5;
or when R¹² is 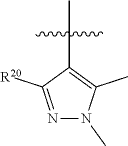
and R¹⁰ is —O-naphthyl substituted with —O— or —S—, then R²⁰ is 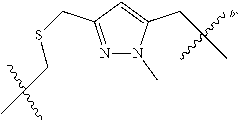 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
Z₂ is N or C, wherein when Z₂ is N, then [symbol] is a single bond; and when Z₂ is C, then [symbol] is a double bond,
and wherein Formula (A) contains a single R¹⁹;
and wherein [linker] has the following formula 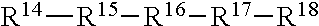
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl-C(O)—, cycloalkyl or CH₂—NH—C(O)—.

98. A compound of formula (I) 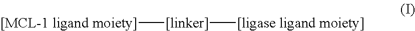
or a salt, solvate, hydrate, isomer or prodrug thereof,
wherein [ligase ligand moiety] is:
(a) Formula (VIa) or (VIb): 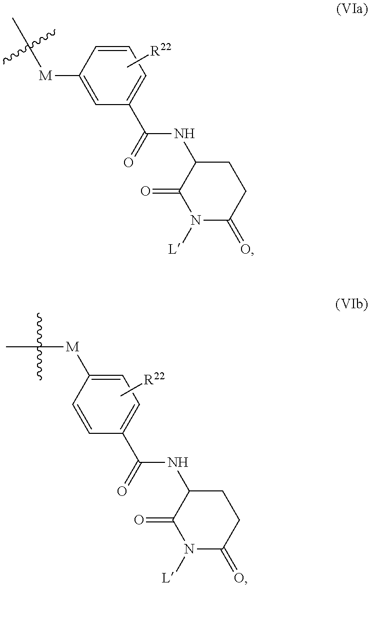
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl; and
L′ is hydrogen, alkyl, benzyl, acetyl or pivaloyl
(b) Formula (II): 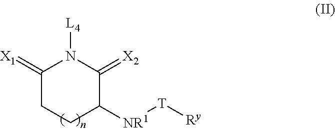
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from 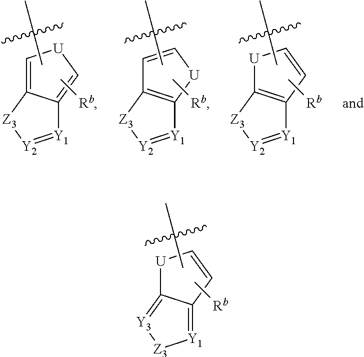
wherein [symbol] indicates attachment to T,
Z₃ is O, S or NR³;
U is O, S, NR^{b} or CRⁱ₂;
each of Y₁, Y₂ and Y₃ is independently N or CR^{d};
each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
each R^{b} is independently alkyl, cycloalkyl, alkenyl, cycloalkenyl, aryl, heteroaryl, or benzyl;
R²¹ is a bond connected to R¹⁸ of the linker, wherein Formula (II) contains a single R²¹;
wherein,
(i) when R^{y} is 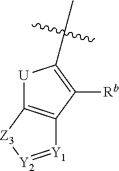
then Y₂ is CR^{d}; and
(ii) when R^{y} is 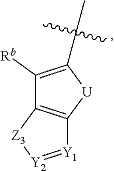
then Rⁱ in CRⁱ₂ is not hydrogen
or
(c) Formula (III): 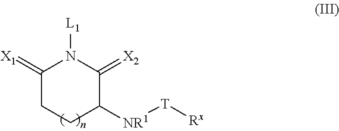
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from 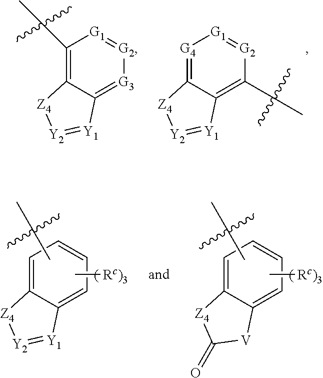
wherein [symbol] indicates attachment to T,
Z₄ is O, S or NR⁴;
V is CR^{f}₂, NR⁴ or S;
each of G₁, G₂, G₃ and G₄ is independently N or CR^{C},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f} then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{C}, then C═X₁ may be replaced by CH;
and wherein:
(i) when R^{x} is 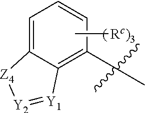
and Z₄ is NH, then L₁ is hydrogen, —CH₂C(O)OR^{b}, or —OR^{b};
(ii) when R^{x} is 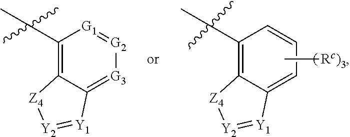
Z₄ is NR⁴, Y₁ is CR^{f}, and Y₂ is N, then R⁴ is not alkyl and at least one of R² and R is not H;
(iii) when R^{x} is 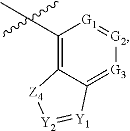
Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not 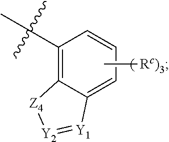
(v) when R^{x} is 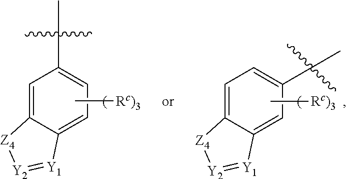
Z₄ is NR⁴, and Y₁ or Y₂ is N, then R⁴ is not alkyl;
(vi) when R^{x} is 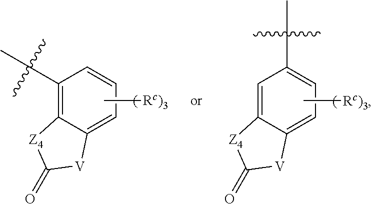
then n=1 or 2; and
(vii) when R^{x} is 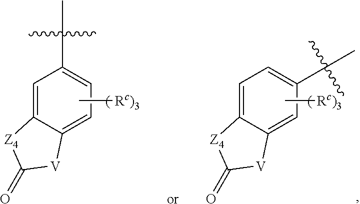
then Z₄=O or S
wherein [MCL-1 ligand moiety] is a compound of Formula (A): 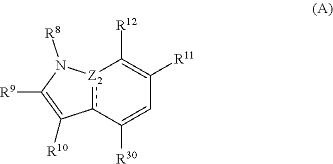
wherein [symbol]
is a single bond or a double bond;
R⁸ is H, R¹⁹, or C₁-C₆ alkyl optionally substituted with morpholine or a piperazine;
R⁹ is —C(O)OH, —C(O)OC₁-C₆alkyl, —C(O)NH₂ or —P(O)(OH)₂,
one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl,
R¹² is H, 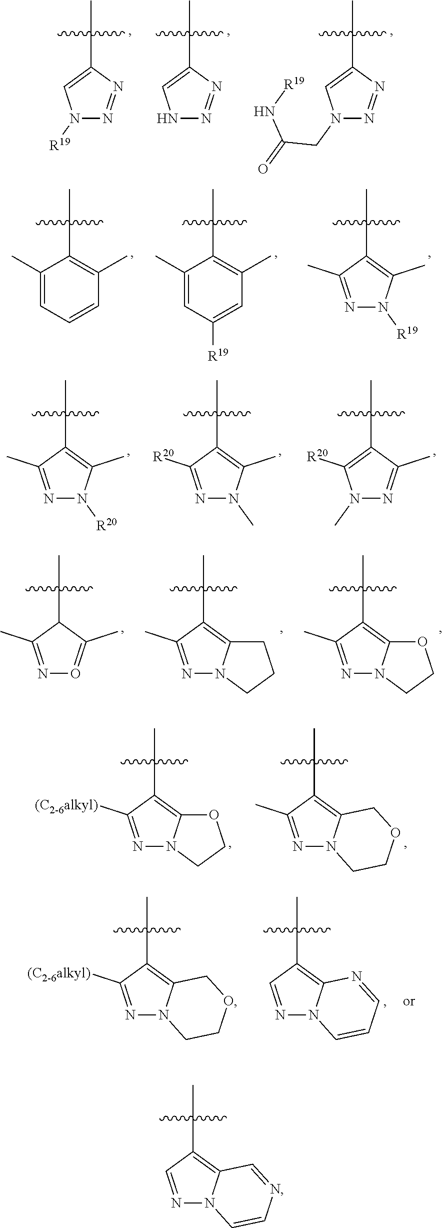
wherein R²⁰ s Me, —CH₂—OMe, —(CH₂CH₂O)ₚ(C₁-C₆ alkyl), —CH₂—O-bromobenzaldehyde, or 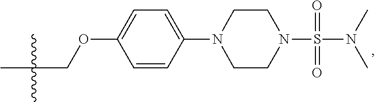
wherein p is 1-5; or
when R¹² is 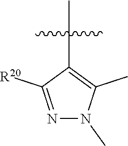
and R¹⁰ is —O-naphthyl substituted with —O— or —S—, then R²⁰ is 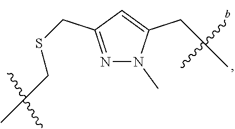 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
Z₂ is N or C, wherein when Z₂ is N, then [symbol] is a single bond; and when Z₂ is C, then [symbol] is a double bond,
and wherein Formula (A) contains a single R¹⁹;
and wherein [linker] has the following formula 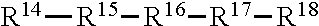
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl-C(O)—, cycloalkyl or CH₂—NH—C(O)—.

99. The compound of claim 97 or 98, wherein
R¹⁶ is —C₁₋₆ alkyl, —C(O)—, C(O)—NH—, —C(O)O—, —CH₂—C(O)—, —CH₂—C(O)—NH—, —CH₂—C(O)O— or is absent
R¹⁷ is —CH₂(C₂H₄—O)_{y}, (C₂H₄—O)ₓ, (C₃H₆—O)ₓ, or is absent; and
R¹⁸ is cycloalkyl or CH₂—NH—C(O)—.

100. The compound of any one of claims 59-99, wherein one of R¹⁰ and R³⁰ is H and the other of R¹⁰ and R³⁰ is —C₂₋₅alkyl-O—R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the naphthyl is optionally substituted with —O— or —S—.

101. The compound of any one of claims 59-100, wherein R³⁰ is H.

102. The compound of any one of claims 59-101, wherein R¹² is H,

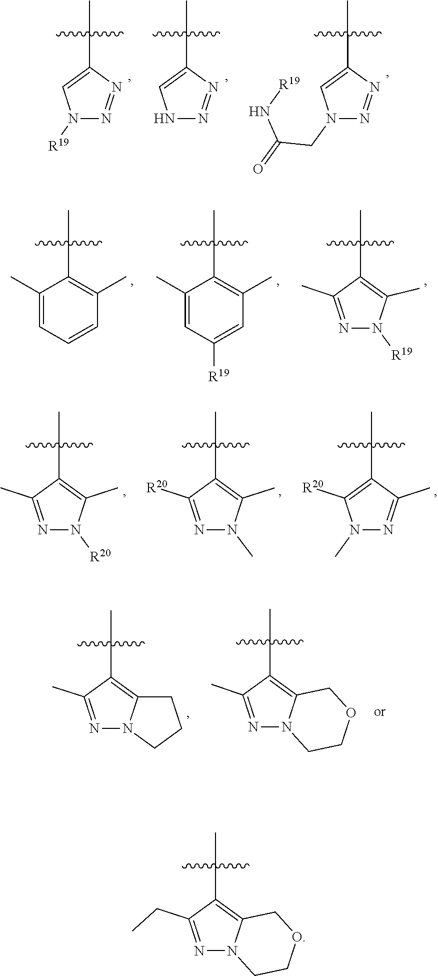

103. The compound of any one of claims 59-102, wherein R²⁰ is Me, —CH₂—O-bromobenzaldehyde, or

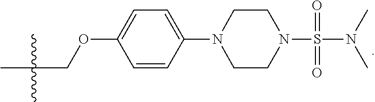 [SMILES: *COc1ccc(N2CCN(S(=O)(=O)N(C)C)CC2)cc1]

104. The compound of any one of claims 59-103, wherein Z₂ is C and [symbol] is a double bond.

105. The compound of any one of claims 59-104, wherein R¹¹ is hydrogen.

106. The compound of any one of claims 59-104, wherein R¹¹ is halogen.

107. The compound of any one of claims 59-106, wherein [MCL-1 ligand moiety] is:

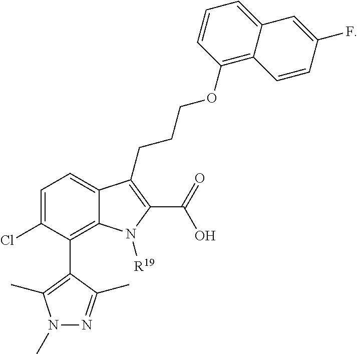

108. A compound of formula (I) 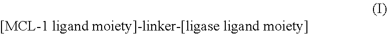
or a salt, solvate, hydrate, isomer or prodrug thereof,
wherein [ligase ligand moiety] is: 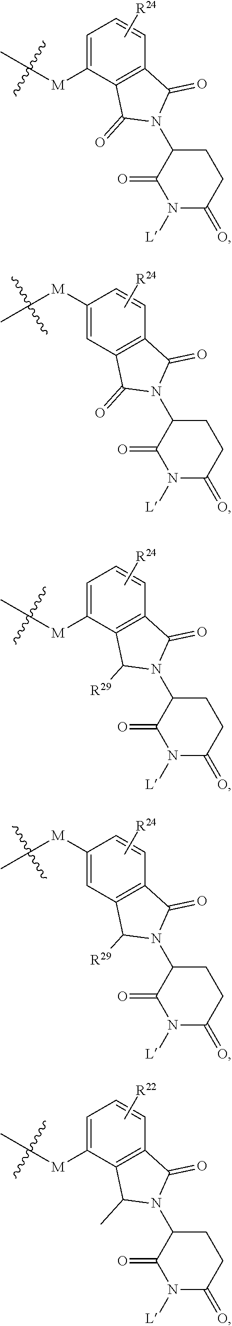 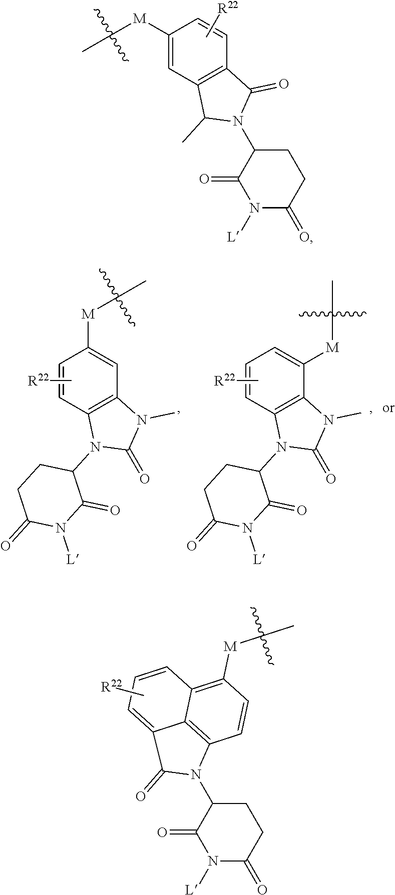
wherein
M is O, S or NH, or is absent; [symbol]
indicates attachment to R¹⁸ of the linker;
R²⁴ is —OMe or heterocycloalkyl
R²² is hydrogen, halogen, —OMe, an amino group, heterocycloalkyl, or unsubstituted C₁-C₆ alkyl;
R²⁹ is hydrogen or Me; and
L′ is hydrogen, alkyl, benzyl, acetyl or pivaloyl;
wherein [MCL-1 ligand moiety] is a compound of Formula (A): 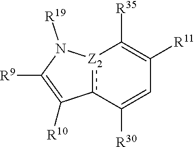
wherein [symbol]
is a single bond or a double bond;
R⁸ is H, R¹⁹, or C₁-C₆ alkyl optionally substituted with morpholine or a piperazine;
R⁹ is —C(O)OH, —C(O)OC₁-C₆alkyl, —C(O)NH₂ or —P(O)(OH)₂,
one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl);
or wherein the tetraline is optionally substituted with a bridging —CH₂— group; or wherein the naphthyl is optionally substituted with —O— or —S—,
R¹¹ is H, halogen or C₁-C₆ alkyl,
R¹² is H, 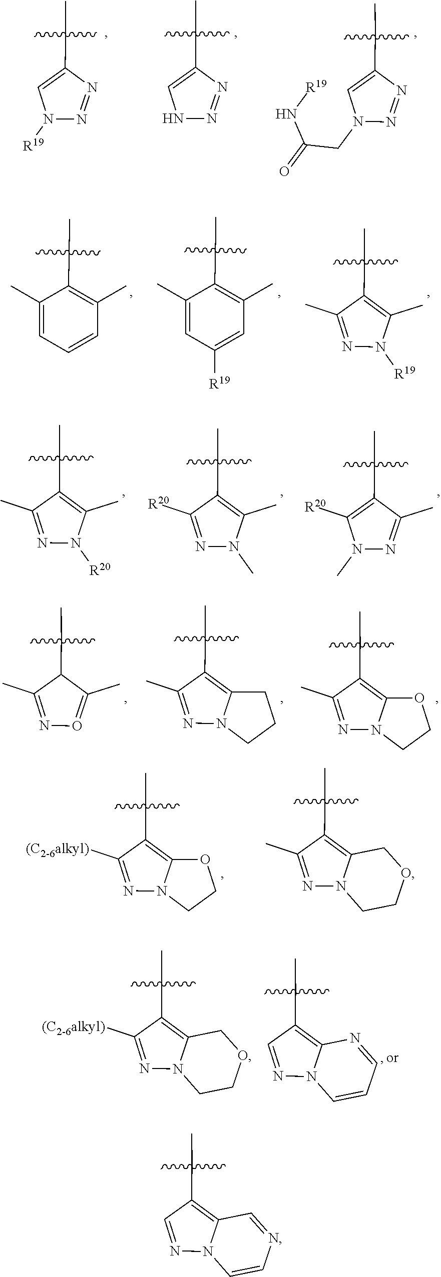
wherein R²⁰ is Me, —CH₂—OMe, —(CH₂CH₂O)ₚ(C₁-C₆ alkyl), or —CH₂—O-bromobenzaldehyde, wherein p is 1-5;
or when R¹² is 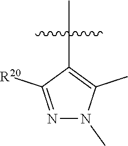
and R¹⁰ is —O-naphthyl substituted with —O— or —S—, then R²⁰ is 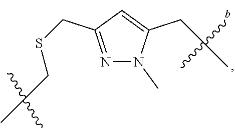 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
Z₂ is N or C, wherein when Z₂ is N, then [symbol] is a single bond; and when Z₂ is C, then [symbol] is a double bond,
and wherein Formula (A) contains a single R¹⁹;
and wherein [linker] has the following formula 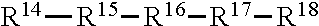
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl, —C₁₋₆ alkyl-C(O)—, cycloalkyl, CH₂—NH—C(O)—, heterocycloalkyl, or is absent
wherein at least one of R¹⁴-R¹⁸ is present

109. The compound of claim 108, wherein L′ is hydrogen or methyl.

110. The compound of claim 109, wherein L′ is hydrogen.

111. The compound of any one of claims 108-109, wherein M is O or NH, or is absent.

112. The compound of claim 108, wherein [ligase ligand moiety] is

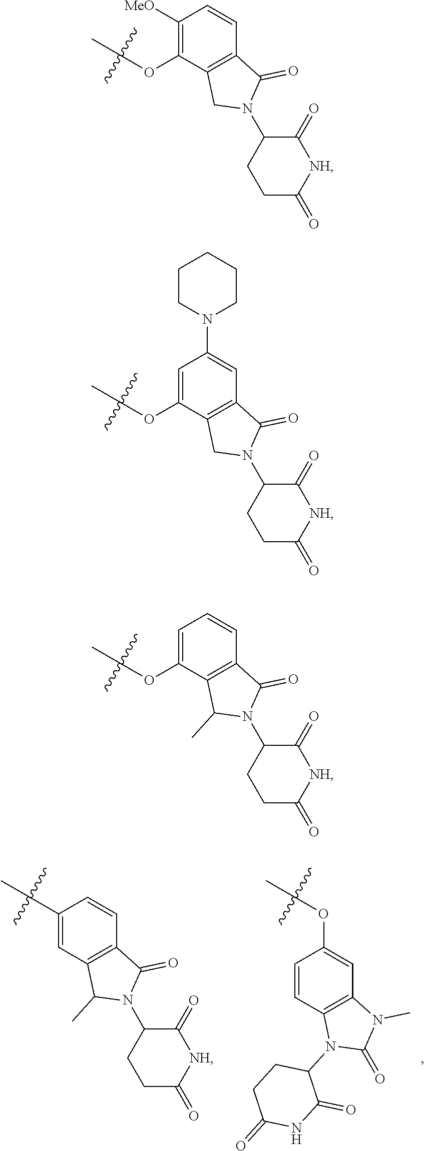 [SMILES: *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cccc2c1C(C)N(C1CCC(=O)NC1=O)C2=O ; *c1ccc2c(c1)C(C)N(C1CCC(=O)NC1=O)C2=O ; *Oc1ccc2c(c1)n(C)c(=O)n2C1CCC(=O)NC1=O]

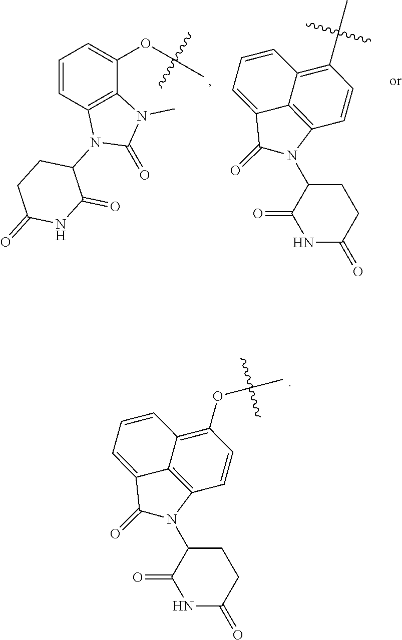 [SMILES: *Oc1cccc2c1n(C)c(=O)n2C1CCC(=O)NC1=O ; *c1ccc2c3c(cccc13)C(=O)N2C1CCC(=O)NC1=O ; *Oc1ccc2c3c(cccc13)C(=O)N2C1CCC(=O)NC1=O]

113. The compound of any one of claims 108-112, wherein:
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent;
R¹⁷ is —CH₂(C₂H₄—O)_{y}, (C₂H₄—O)ₓ, (C₃H₆—O)ₓ, or is absent; and
R¹⁸ is C₁₋₆ alkyl, cycloalkyl, CH₂—NH—C(O)—, heterocycloalkyl, or is absent.

114. The compound of any one of claims 108-113, wherein R¹⁴ is —C₁₋₆ alkyl, —C₂₋₆ alkenyl, —C₂₋₆ alkynyl, —C(O)—, —SO₂— or is absent

115. The compound of claim 114, wherein R¹⁴ is —C₁₋₆alkyl.

116. The compound of any one of claims 108-115, wherein R¹⁵ is cycloalkyl, heterocycloalkyl, aryl, heteroaryl, —C₁₋₆ alkyl-NH—, -cycloalkyl-NH— or is absent.

117. The compound of claim 116, wherein R¹⁵ is heterocycloalkyl or is absent.

118. The compound of claim 117, wherein R¹⁵ is piperazine, bridged piperazine, piperazine N-oxide, piperazine cation, 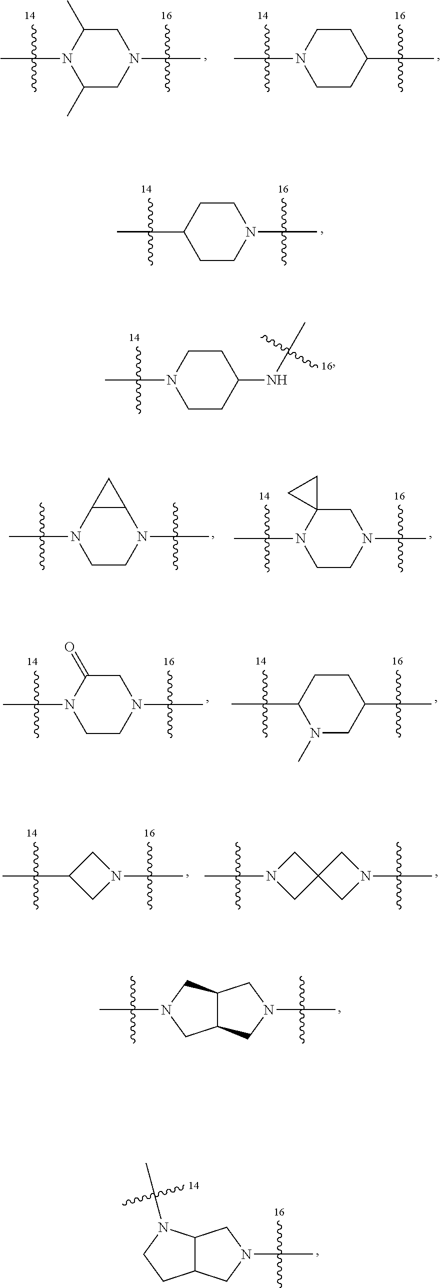 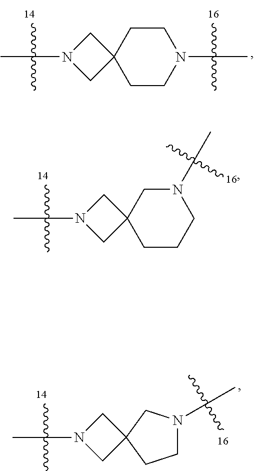 [SMILES: *N1CCC2(CC1)CN(*)C2 ; *N1CCCC2(C1)CN(*)C2 ; *N1CCC2(C1)CN(*)C2]
or is absent;
wherein [symbol] indicates attachment to R¹⁴ and [symbol] indicates attachment to R¹⁶.

119. The compound of claim 118, wherein R¹⁵ is piperazine, 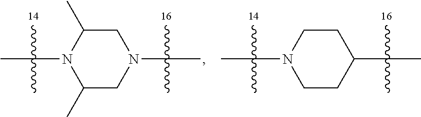
or is absent.

120. The compound of any one of claims 108-119, wherein R¹⁶ is —C₁₋₆alkyl, —CH₂—C(O)—NH—, —CH₂—C(O)—, —C(O)— or is absent.

121. The compound of any one of claims 108-120, wherein R¹⁷ is absent.

122. The compound of any one of claims 108-121, wherein R¹⁸ is —C₁₋₆ alkyl, cyclobutyl, CH₂—NH—C(O)—, piperazine or is absent.

123. The compound of any one of claims 108-122, wherein [linker] is selected from 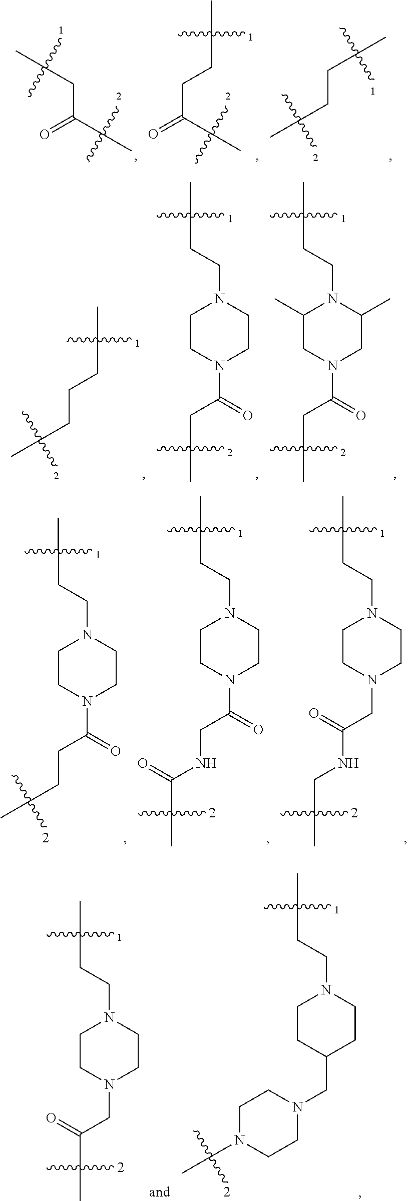
wherein [symbol]
indicates attachment to [MCL-1 ligand moiety] and [symbol]
indicates attachment to [ligase ligand moiety].

124. The compound of claim 123, wherein [linker] is selected from

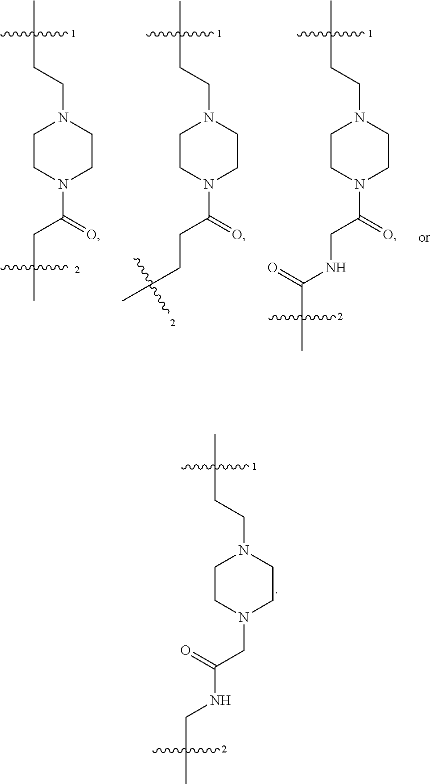 [SMILES: *CCN1CCN(C(=O)C*)CC1 ; *CCC(=O)N1CCN(CC*)CC1 ; *CCN1CCN(C(=O)CNC(*)=O)CC1 ; *CCN1CCN(CC(=O)NC*)CC1]

125. The compound of any one of claims 108-124, wherein [MCL-1 ligand moiety] is selected from:

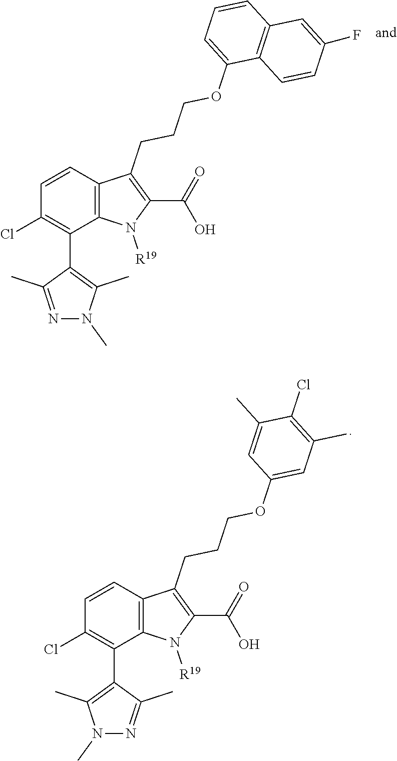

126. The compound of claim 108, which is selected from:

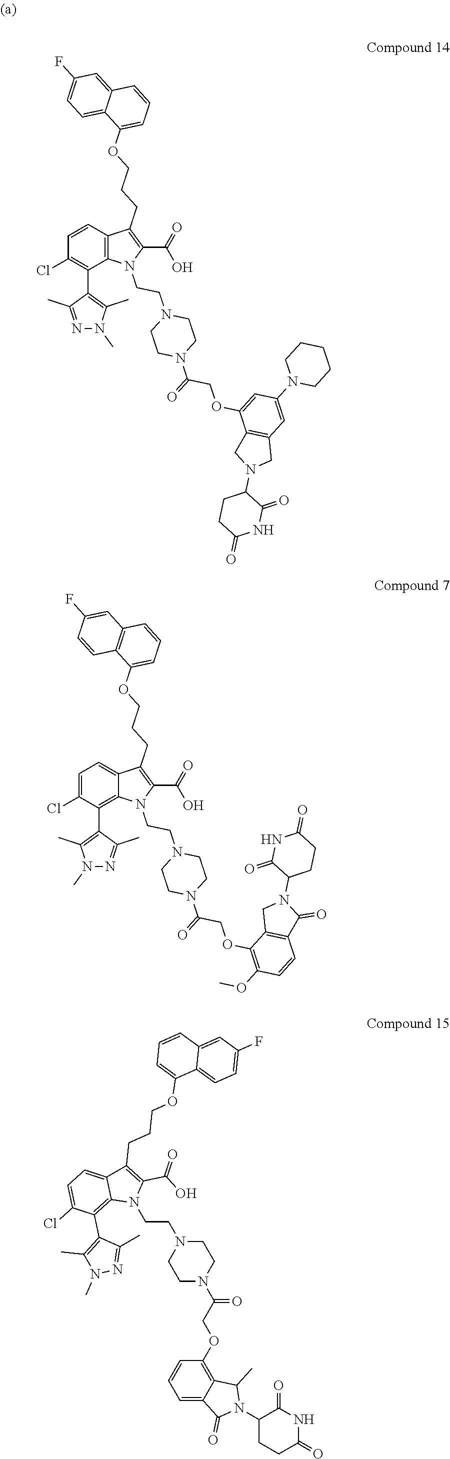 [SMILES: Cc1nn(C)c(C)c1-c1c(Cl)ccc2c(CCCOc3cccc4cc(F)ccc34)c(C(=O)O)n(CCN3CCN(C(=O)COc4cc(N5CCCCC5)cc5c4CN(C4CCC(=O)NC4=O)C5)CC3)c12 ; COc1ccc2c(c1OCC(=O)N1CCN(CCn3c(C(=O)O)c(CCCOc4cccc5cc(F)ccc45)c4ccc(Cl)c(-c5c(C)nn(C)c5C)c43)CC1)CN(C1CCC(=O)NC1=O)C2=O ; Cc1nn(C)c(C)c1-c1c(Cl)ccc2c(CCCOc3cccc4cc(F)ccc34)c(C(=O)O)n(CCN3CCN(C(=O)COc4cccc5c4C(C)N(C4CCC(=O)NC4=O)C5=O)CC3)c12]

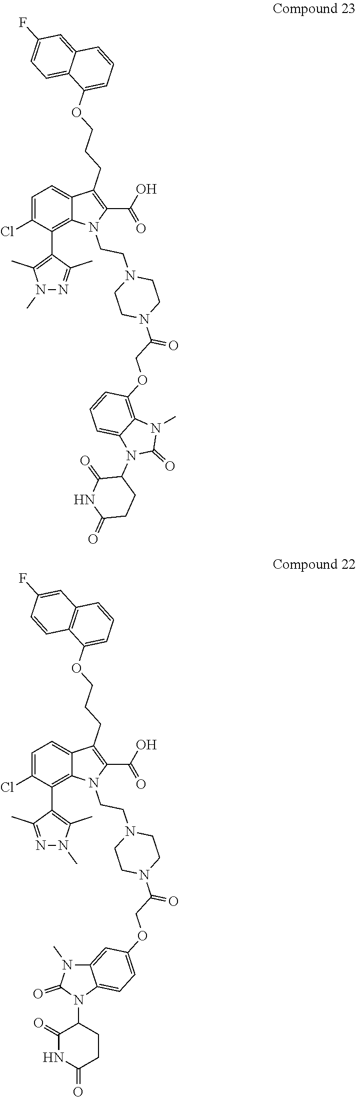

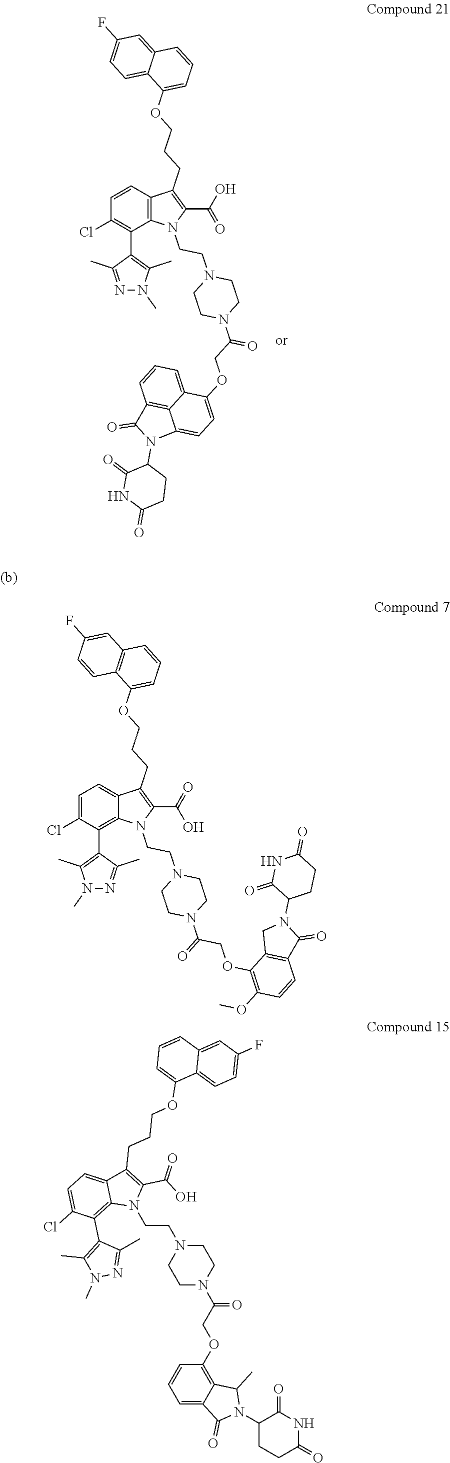 [SMILES: Cc1nn(C)c(C)c1-c1c(Cl)ccc2c(CCCOc3cccc4cc(F)ccc34)c(C(=O)O)n(CCN3CCN(C(=O)COc4ccc5c6c(cccc46)C(=O)N5C4CCC(=O)NC4=O)CC3)c12 ; COc1ccc2c(c1OCC(=O)N1CCN(CCn3c(C(=O)O)c(CCCOc4cccc5cc(F)ccc45)c4ccc(Cl)c(-c5c(C)nn(C)c5C)c43)CC1)CN(C1CCC(=O)NC1=O)C2=O ; Cc1nn(C)c(C)c1-c1c(Cl)ccc2c(CCCOc3cccc4cc(F)ccc34)c(C(=O)O)n(CCN3CCN(C(=O)COc4cccc5c4C(C)N(C4CCC(=O)NC4=O)C5=O)CC3)c12]

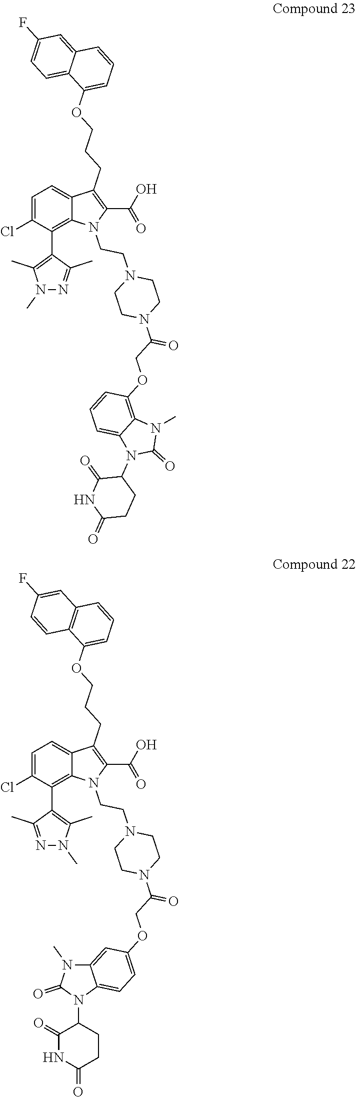

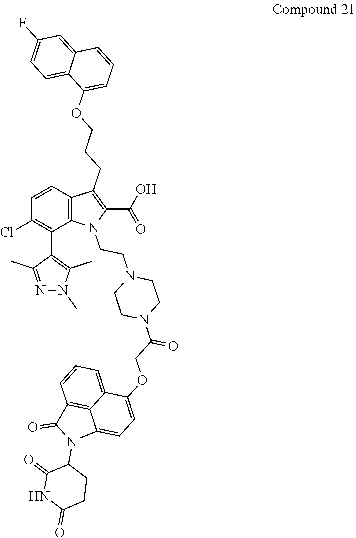 [SMILES: Cc1nn(C)c(C)c1-c1c(Cl)ccc2c(CCCOc3cccc4cc(F)ccc34)c(C(=O)O)n(CCN3CCN(C(=O)COc4ccc5c6c(cccc46)C(=O)N5C4CCC(=O)NC4=O)CC3)c12]

127. The compound of claim 108, wherein the compound is Compound 22.

128. A compound of formula (I) 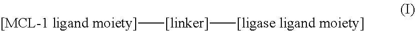
or a salt, solvate, hydrate, isomer or prodrug thereof,
wherein [ligase ligand moiety] is of Formula (VIa) or (VIb): 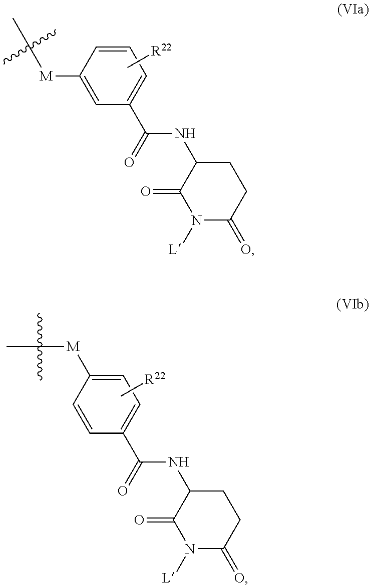
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl; and
L′ is hydrogen, alkyl, benzyl, acetyl or pivaloyl
wherein [MCL-1 ligand moiety] is a compound of Formula (A): 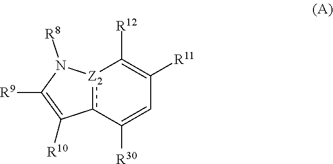
wherein [symbol]
is a single bond or a double bond;
R⁸ is H, R¹⁹, or C₁-C₆ alkyl optionally substituted with morpholine or a piperazine;
R⁹ is —C(O)OH, —C(O)OC₁-C₆alkyl, —C(O)NH₂ or —P(O)(OH)₂,
one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹¹ is H, halogen or C₁-C₆ alkyl, 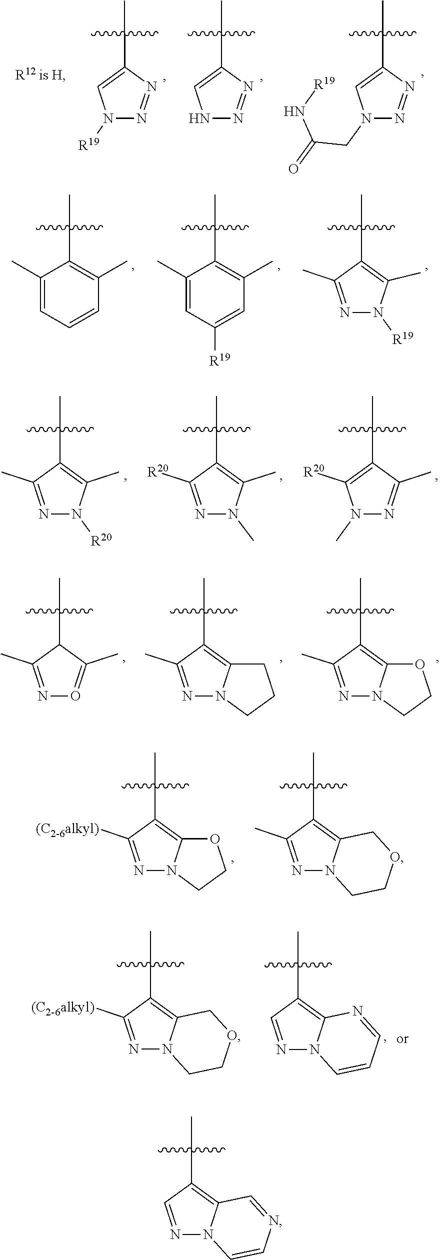
wherein R²⁰ s Me, —CH₂—OMe, —(CH₂CH₂O)ₚ(C₁-C₆ alkyl), —CH₂—O-bromobenzaldehyde, or 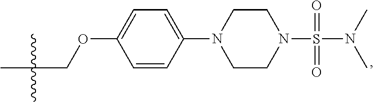
wherein p is 1-5; or
when R¹² is 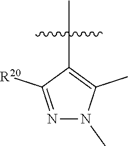
and R¹⁰ is —O-naphthyl substituted with —O— or —S—, then R²⁰ is 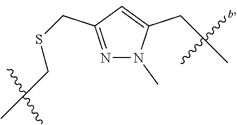 [SMILES: *CSCc1cc(C*)n(C)n1]
wherein [symbol] indicates attachment to —O— or —S— of R¹⁰;
and wherein
R¹⁹ is a bond connected to R¹⁴ of the linker;
Z₂ is N or C, wherein when Z₂ is N, then [symbol] is a single bond; and when Z₂ is C, then [symbol] is a double bond,
and wherein Formula (A) contains a single R¹⁹;
and wherein [linker] has the following formula 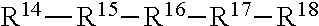
wherein
R¹⁴ is —C₁₋₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, (C₂H₄—O)ₓ, (C₃H₆—O)ₓ, or is absent
x is 1-10
y is 2-10
R¹⁸ is —C₁₋₆ alkyl, —C₁₋₆ alkyl-C(O)—, cycloalkyl, CH₂—NH—C(O)—, heterocycloalkyl, or is absent
wherein at least one of R¹⁴-R¹⁸ is present.

129. The compound of claim 128, wherein [ligase ligand moiety] is:

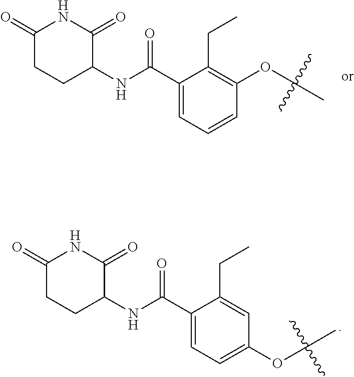 [SMILES: *Oc1cccc(C(=O)NC2CCC(=O)NC2=O)c1CC ; *Oc1ccc(C(=O)NC2CCC(=O)NC2=O)c(CC)c1]

130. The compound of claim 128 or 129, wherein
R¹⁶ is —C₁₋₆ alkyl, —C(O)—, —C(O)—NH—, —C(O)O—, —CH₂—C(O)—, —CH₂—C(O)—NH—, —CH₂—C(O)O— or is absent;
R¹⁷ is —CH₂(C₂H₄—O)_{y}, (C₂H₄—O)ₓ, (C₃H₆—O)ₓ, or is absent; and
R¹⁸ is —C₁₋₆ alkyl, cycloalkyl, CH₂—NH—C(O)—, heterocycloalkyl, or is absent.

131. The compound of any one of claims 128-130, wherein R¹⁴ is —C₁₋₆ alkyl, —C₂₋₆ alkenyl, —C₂₋₆ alkynyl, —C(O)—, —SO₂— or is absent

132. The compound of claim 131, wherein R¹⁴ is —C₁₋₆alkyl.

133. The compound of any one of claims 128-132, wherein R¹⁵ is cycloalkyl, heterocycloalkyl, aryl, heteroaryl, —C₁₋₆ alkyl-NH—, -cycloalkyl-NH— or is absent.

134. The compound of claim 133, wherein R¹⁵ is heterocycloalkyl or is absent.

135. The compound of claim 134, wherein R¹⁵ is piperazine.

136. The compound of any one of claims 128-135, wherein R¹⁶ is —C(O)—.

137. The compound of any one of claims 128-136, wherein R¹⁷ is absent.

138. The compound of any one of claims 128-137, wherein R¹⁸ is —C₁₋₆ alkyl.

139. The compound of any one of claims 128-138, wherein [linker] is

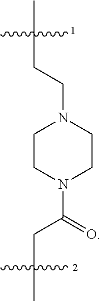 [SMILES: *CCN1CCN(C(=O)C*)CC1]

140. The compound of any one of claims 128-139, wherein [MCL-1 ligand moiety] is

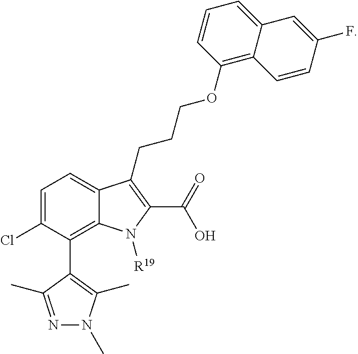

141. The compound of claim 140, wherein the compound is:

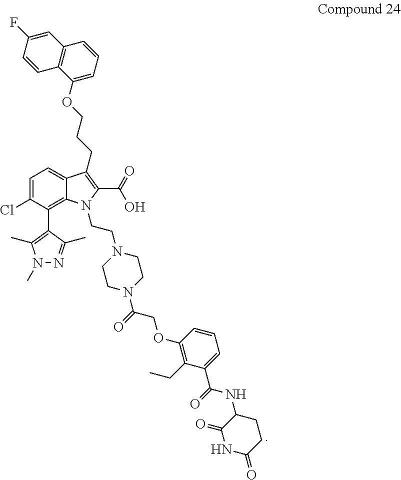 [SMILES: CCc1c(OCC(=O)N2CCN(CCn3c(C(=O)O)c(CCCOc4cccc5cc(F)ccc45)c4ccc(Cl)c(-c5c(C)nn(C)c5C)c43)CC2)cccc1C(=O)NC1CCC(=O)NC1=O]

142. The compound of any preceding claim, wherein T is C═O.

143. The compound of any preceding claim, wherein:
(a) X₁ and X₂ are O;
(b) X₁ is O and X₂ is S;
(c) X₁ is S and X₂ is O; or
(d) X₁ and X₂ are S.

144. The compound of any preceding claim, wherein n is 1.

145. The compound of any preceding claim, wherein unless otherwise specified each alkyl, alkenyl, alkynyl, aryl, heteroaryl and benzyl is unsubstituted.

146. A compound selected from:

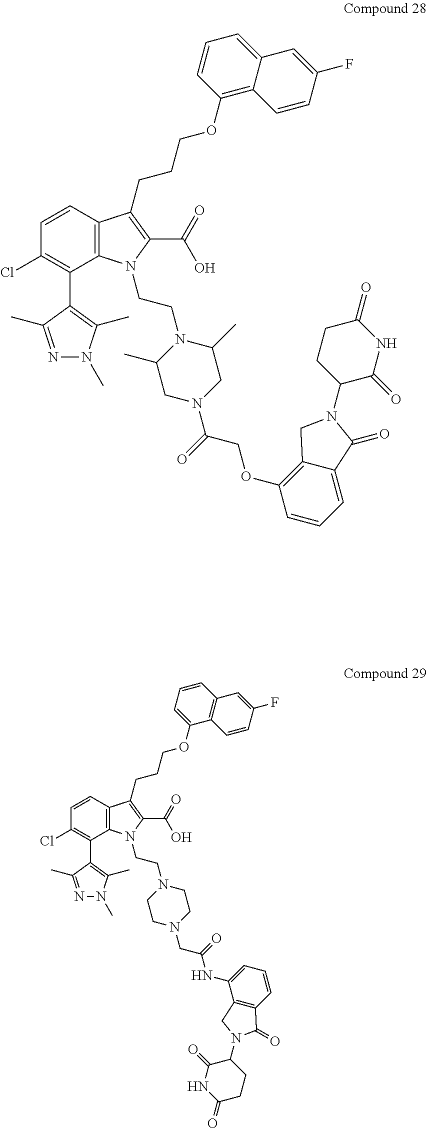 [SMILES: Cc1nn(C)c(C)c1-c1c(Cl)ccc2c(CCCOc3cccc4cc(F)ccc34)c(C(=O)O)n(CCN3C(C)CN(C(=O)COc4cccc5c4CN(C4CCC(=O)NC4=O)C5=O)CC3C)c12 ; Cc1nn(C)c(C)c1-c1c(Cl)ccc2c(CCCOc3cccc4cc(F)ccc34)c(C(=O)O)n(CCN3CCN(CC(=O)Nc4cccc5c4CN(C4CCC(=O)NC4=O)C5=O)CC3)c12]

147. A compound selected from:

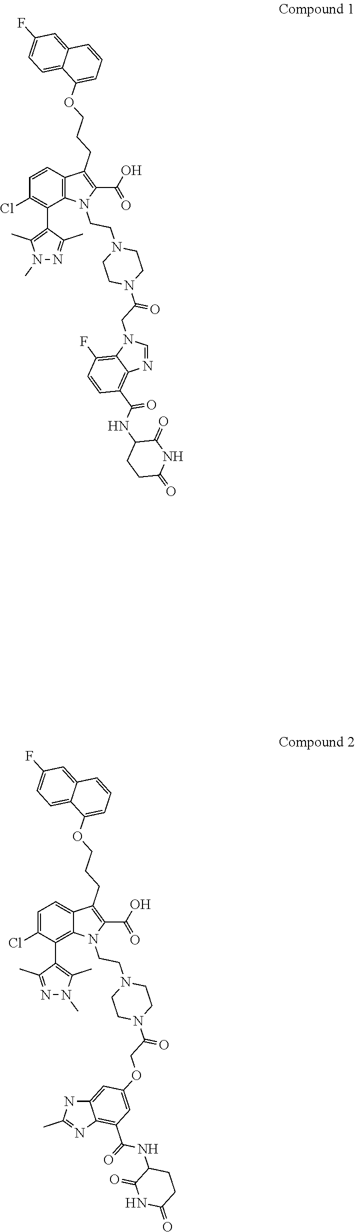 [SMILES: Cc1nn(C)c(C)c1-c1c(Cl)ccc2c(CCCOc3cccc4cc(F)ccc34)c(C(=O)O)n(CCN3CCN(C(=O)Cn4cnc5c(C(=O)NC6CCC(=O)NC6=O)ccc(F)c54)CC3)c12 ; Cc1nc2c(C(=O)NC3CCC(=O)NC3=O)cc(OCC(=O)N3CCN(CCn4c(C(=O)O)c(CCCOc5cccc6cc(F)ccc56)c5ccc(Cl)c(-c6c(C)nn(C)c6C)c54)CC3)cc2[nH]1]

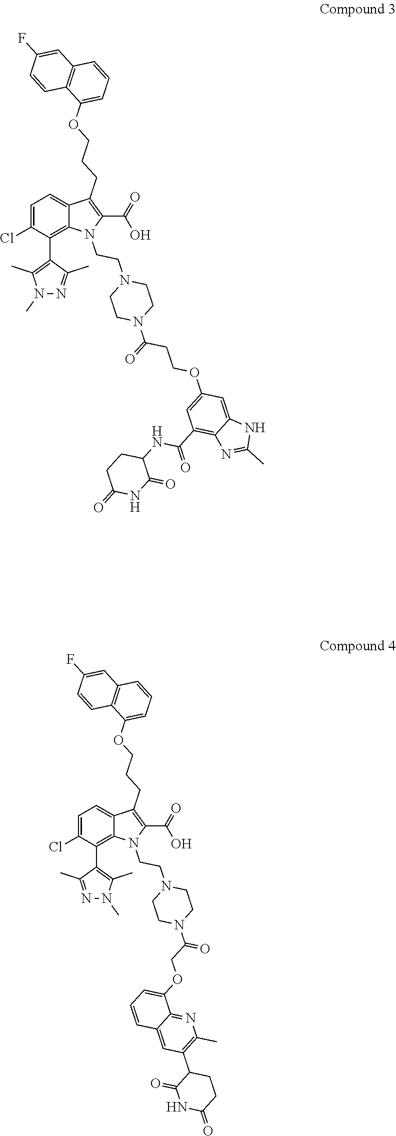

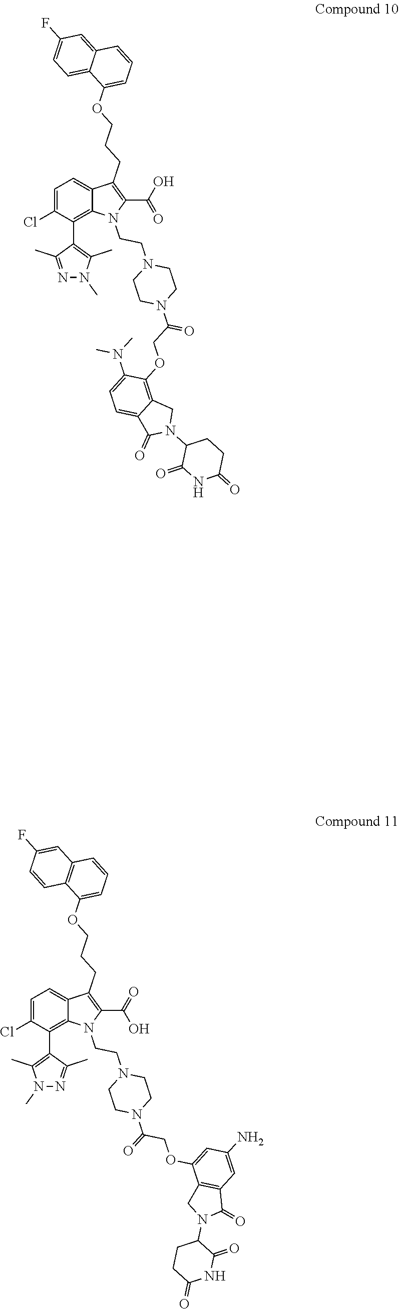 [SMILES: Cc1nn(C)c(C)c1-c1c(Cl)ccc2c(CCCOc3cccc4cc(F)ccc34)c(C(=O)O)n(CCN3CCN(C(=O)COc4c(N(C)C)ccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)O)n(CCN3CCN(C(=O)COc4cc(N)cc5c4CN(C4CCC(=O)NC4=O)C5=O)CC3)c12]

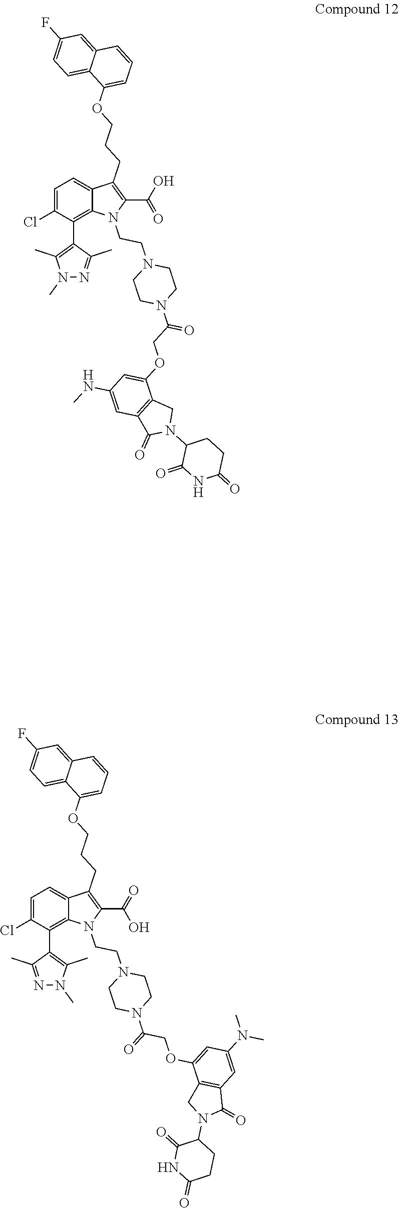

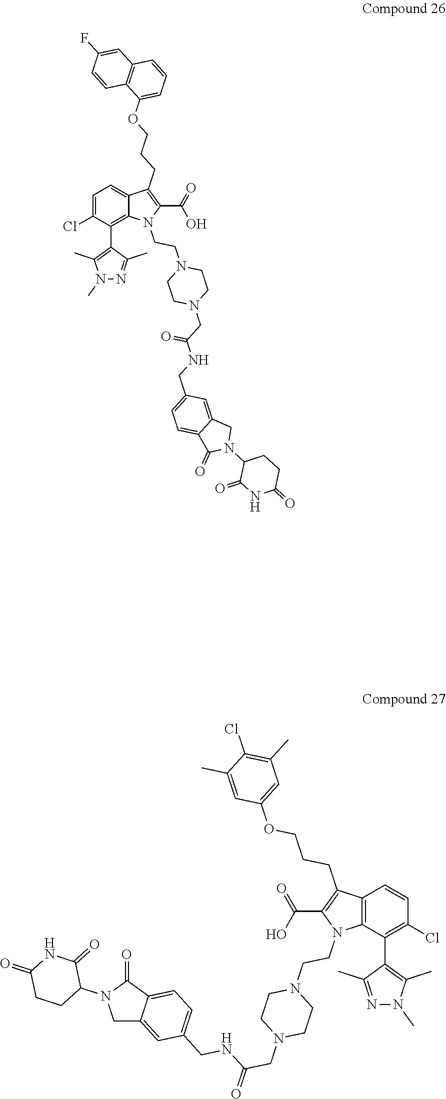 [SMILES: Cc1nn(C)c(C)c1-c1c(Cl)ccc2c(CCCOc3cccc4cc(F)ccc34)c(C(=O)O)n(CCN3CCN(CC(=O)NCc4ccc5c(c4)CN(C4CCC(=O)NC4=O)C5=O)CC3)c12 ; Cc1cc(OCCCc2c(C(=O)O)n(CCN3CCN(CC(=O)NCc4ccc5c(c4)CN(C4CCC(=O)NC4=O)C5=O)CC3)c3c(-c4c(C)nn(C)c4C)c(Cl)ccc23)cc(C)c1Cl]

148. A compound of claim 147, selected from: [chemical structure] [chemical structure]
optionally wherein the compound is Compound 12.

149. A pharmaceutical composition comprising a compound of any one of claims 1-148.

150. The compound of any one of claims 1-148 or the pharmaceutical composition of claim 149, for use in medicine.

151. A method of treating cancer in a subject in need thereof, the method comprising administering to the subject an effective amount of a compound according to any one of claims 1-148, or a pharmaceutical composition according to claim 149.

152. The method of claim 151, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.

153. The method of claim 151, wherein the cancer is multiple myeloma acute myeloid leukaemia.

154. The method of any one of claims 151-153, wherein the administration does not result in cytotoxicity in cardiomyocytes in the subject.

155. The method of any one of claims 151-154, further comprising administering at least one additional active agent to the subject.

156. The method of claim 155, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

157. A method of reversing resistance to chemotherapy or targeted cancer therapies in a subject in need thereof, the method comprising administering to the subject an effective amount of a compound according to any one of claims 1-148, or a pharmaceutical composition according to claim 149.

158. A combined preparation of a compound of any one of claims 1-148 and at least one additional active agent, for simultaneous, separate or sequential use in therapy.

159. The combined preparation of claim 158, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

160. The combined preparation of any one of claims 158-159 wherein the therapy is the treatment of cancer.

161. A method of reducing the cardiac cytotoxicity of an MCL-1 inhibitor, comprising coupling a cereblon binding moiety to the MCL-1 inhibitor, wherein the cereblon binding moiety is a [ligase ligand moiety] as defined in any one of claims 1-148 and the MCL-1 inhibitor is an [MCL-1 ligand moiety] as defined in any one of claims 1-148.
